SEQUENCE LISTING (628 sequences)

SEQ ID NO: 1 - protein, 924 aa, unidentified
  REGION 1..924: note = uncultivated organism
  REGION 1..924: note = MG122-1 Effector
MKLNHLSIAQRMEGAENIHELKENKKTISCGCAAMVDTIESADYIIRLHKAAIAAQNWTVQELGKIMFLPATFISGEGIAEKVTIFDLLSMNKASHKAFASKFSNNGEGFTGTRPDGTPFRTTKSFLGYLSREITSNENSPFCTDMQKRIITSLPEEFQGTASVFIRESIIKIAGSKIEAAEQKKMGPYDNGHAATWCMTDGELSEYIAETEKSAETKRDDEKKTPKKQDNSRQDNTWKRSVRFALADLDTITASITGFAGISSTELAILKEQLLITGPDPHAVDNGRVPGPVVWPAGRKVKVGNEVVTRNVKRPEWDGVFKAVYLNALDRFKTAVIENLPNITRGGLPIPVNIDYSERDERIAAGKKYDSKLIPIHLPMNKSCKVAIPEIFIKTRSGHSTDGYFPHNFTEMVPVTKAICCTLRLPDGCTPETLSSVNSGLIAQISYETVTTAVRMRPKADIPREKILGIDVNSATFKYNTSAGRLQAGQDMVDWHEAIAYFQGICPNDFLFNMGKYCTFEHIHTRICAEEYMASCKARLFSKASKQVDDILAQFRSGEYGIAMMPTLALLPTVFKPDTREVLPKDPMFNLFRALIERKGADGKTPYYSENARDTISHTRRWRHEIALALSAKLEYRDKAITWQADHDPVAEPFTNTEEAASILDRMTAAVNSAKQIEEHILSRSLFSYETVSRFTIYAVEDIDFDCLKDKSVSFMSLASTLRQIPALASGCSVAAQEASGNVRVKLDGGTGAEEAVTEIRKWFAGTSHRGQWSLETAEAVSAGEIRMKCSITRKGRIDRAVATVPNLLTKVMRLPEVKNTVERLCVKNATPFVSVDPRSTSLTCSACGARTTKEACKKGEPFCKETGRPFRLGERFVCHKCGREINANWNAAINVAGRASFIMANAKNKKKLAEPSDNSLPGM

SEQ ID NO: 2 - protein, 913 aa, unidentified
  REGION 1..913: note = uncultivated organism
  REGION 1..913: note = MG122-2 Effector
MKHLTVKDRTKGKSGSIFKIDTTTETVSCGCPAMVDSVESAEYLARLYTAAINAQNYAISMVRDMLFVPAKFVAPDGHAEKVTPFDLFAMNGPTARAFFERFATVETVGGEKTSYFAGVRPDRKKFRTVGSFMQYLSREIFSDTSSPFGKALKTKIIESMPGEFRSRSVSFVNESVIKVAIPKVACAIRKKVEQYTGSCVEPWCLSGEEAHKASGKSSGKRADHTWKRIARDVCDSLVDIDPLDKVTLYAQLNAVGPRCHEFINRKNPVVGHWPENATVRIPGLARKMPLDRPSWNGQFRRVYGQAIEKFMDEVAARLPKITGGSLVVLGEVDGDGRESRIRRGDKFDCVFNVSHVPYDKEKFVPSVKLYLQTRTRHTKDGYFPSNFDETLGTNKVVQCSLRFPSEFDVQSLTRRSSKLIAQLSYERVSDKVRFAGPCELSDKDVVGIDANSSTFKINTTIPPKRKCSDIPDWHEAFLAFRESNRDHFVFNAKQYADGLQTDMQDGLLRSTLMERFAVQVDSIVESAKSGKYGLCLFPTLGIPLMLFENNLYLAGENPGKDPLSALFTFMANRKDANGNPFYGPAAREAIGHTRAWRRATAAALAEKLRYITESRVWQETHDPVNSPFGDTGRAAELSERCSAAMRTARQIEEHVLSKTLFSKNTVSKFKLFALEELDFDGMRSKDGGFVSLGATLRSMLGDMYKVGPCREASGYAEIELPYDDKPAAFTKRLKELFKERKGKCNWEIVKVSYSLKIVGKFEKNNCTRVETAKTYNVAKVKFRLTRKGFCAMNAGLVPAALTKVLHLPELQHTMERLCAKNATSYISVNPKYTSCTCSKCQAKTGKIMCGKGESYCKEMHVPYRRGEYFHCYQCGTEMNANFNAALNVRGSGIRAIGSIGKKKTESGGRRMKK

SEQ ID NO: 3 - protein, 801 aa, unidentified
  REGION 1..801: note = uncultivated organism
  REGION 1..801: note = MG122-3 Effector
MQKTSTSVVYTNVTKKGKPITRTRTIKCPVAFVSKKDAEWVMQLGVATKITMNHVLGILRDKFFDTMTIIAGGKEQVITLFDLFNSKSGLEGPFVYNGNTTCESIVENPVRELLNPKCPLIGVTTKEIIHFMETQLPDGVYVPGKFVKESVYKQVQGTVRGAVTKKLNQEKGKAKKDKTWQKCCRKAAEVHTKTDDAAKELSLQLRIVGVQIYAMNNAGTPQIDEWPKECDVPKPEINEAFIAAYKDAIALFKTAFVEAFPEIRSQSLLYCPMFKLDWGTRDTAKFYDIQGKITGIPNIENKKRYSVKVYIRTVSGHSDNYFPKDLQESLGNESVLGIKFPEVITHEDITSVDGVRATKCNTLGYLSVTCPHMKTKFNADGISTDDGVGIDLNVASFFMNTTIKMDETDGAVDWPEAVNEFRSQNPTAYPFTMPYPRSVKDLNLIADNVNEKDRLLRVIPLVGLRDGKPSDEKHGWKASPDPLSMLFTWMLKRTDNNGNPFYSDEQRAHIGHTRDYRKLIRSDTANRLHYFHEQSKWDLSHDCKVNPFSESEQGQALLAERRSIQNRIEKEFVRLVVTGLFSVVKPERIAYLKMENVDLSQIKDNNKPKSLYMTAHDVWGMDDGKLSSKKNKVEFITEVQYDLRAITSTEYWNVISVTHDGDTVVVNAEPTDKWREESARYWIDAYTHRALHFSSVKHLIEELCIKRCIHFVLIDPAHTSQLCHVCRSVEHINFKGSDTEKLSREECLSKHVNFRYGRTFVCGNPECYMCGKEQDSDDNGAWNVLLPQIVIKRKRVKKSKS

SEQ ID NO: 4 - protein, 827 aa, unidentified
  REGION 1..827: note = uncultivated organism
  REGION 1..827: note = MG122-4 Effector
MKKVKIFSSKSVTRTTTGPDNNPLYRKLTRKFPFAFQDVEDAKWVIRISSGVKDSMNRVLGILRDHFFDPIAATVGGEERVITLFDLFDPGCKLETPFVVDGKTTNKVLSGNYTVREIFNVKCPLVPVDRDALVAEFCRQVTSGEHDSLPYLTVFANEGIFAKVCTPVRVAVNRKLNRVKVANARLRDRTWPTCCREAASAHTTNDTEAAVLSRQVEVVGVQIHALNNGGVPVIKKWPRACKVTVGKTSIPVNEPELDEDFMRVYREAVELFKAAFKKAFPVIRRGVLLEGASISIDREGRDTSLYYDTPGVLSGIPSRPGTQGIRVRFRTVSGRGTGYYGEDILDRITGNPKFGMHIPEDATPEGLCYGEGGLARSGCNAFGHVFTEIPLVETARVPDAENPEEFLDIPEEGVGIDVNVGDFLMNTTVALSETTGAVDWCEAIAAFRAARPDWYPFTRAAHRVVREMHKLADEASRDGRLVRVGLLVGLRDGKPADAAHGWKPAPDPLCTLFHWMIRRTGNDGTPFYTREQLAHIGHTRDYRSLVRKELANYLHYFYEQSKWELKHDPKVEPFCETDAGRKLLKERWAIGDLVEKETARLITTGLLDIAADDRIQYLRMEDVDLSAIRQDRSAKSLFKTAKDEWGMCDGKLSAKNGKVEFVTGCGFNLAAVKDTEYWHVESVTREGDTVKVQASPTAKRVDEEARYWTDYHTKKTLHLSSLKKMVEELCIKRGIHFAVMDPTCTSKICHVCRSVGHINHRGSDTEKFGPEECMRLHLNYRRGTTFICGNPECGMCGVIQDAGDNAAINVRMPNIFVKTKRVSKKAA

SEQ ID NO: 5 - protein, 619 aa, unidentified
  REGION 1..619: note = uncultivated organism
  REGION 1..619: note = MG122-5 Effector
MNTDIETNANIEINKNKETKDMKIQEQKEIDTANVVDNTIKKFRENLRMSYSADSHKLTYYDGMLNQPVLAYNKEKGGFYFLAALSEHEAKDCQLDMTSQRLFKNYFTSALSDNYRQTNKHTLPDGTFKIVGIDLGLRQPFVGVVYEVRVELGQIVSHKFLRYIKANASENAGIKVEILHNLFDQIKALKSLLYETNKYFVTKKEEEIPDSYRPSFDRAAIAYGDILGIDMDMYRAYVHNFNNINELRHDKNWIISTLIKNLNSSPQKKCGKCGNIDNPTSEQCSKCGEPSRIVEKKCGKCGNIIQMEFMGDMIYRCPKCNNSITGIKDLRTEYTRIKKGEKKSYCKKNNYTDWTIFLKKKIILEYISLLRSFSTIGLKTSDTIGIRKNFAKTLYRWHKGLVRKCYLLSADIINVVRKEGAKVLFLEDLDIKTSIADNREENKLKALWGWGEIKKHLGHQARKHKIAVVAVNPHLTSLVHYETGLKCIETEGRMVTTIKDGKKICIDRDENAAHNIAIRGLYRHANLREFSVDQIDENVYRLNYSQYDGKQKKGALFHHLRTLKCAFVRDNDGVLIKKDLTPEEEKKGEINKKRTYIILHGDKWFLRHEIDEILERELR

SEQ ID NO: 6 - protein, 529 aa, unidentified
  REGION 1..529: note = uncultivated organism
  REGION 1..529: note = MG120-1 Effector
MSEHFITSIPLYVNEYAYSVLTKRFNVAHHIYNCFLGECKKRRDKYLKSDLYKEAGTKYKEFAAKKVGDKKEINELFKSAITESGFYLRSNSHGRDNSLEQFAASTIQRTWLGDHIDSHCRNKLMERAFKAAQRSLKKELNKRTGGYERPKLRFKRFGRDYICSLEGKNSISPLKFEFKNEIYQVVWRGLSLPVYFDENDDTIKHAINNIENISYIRLKRKPVRGKWQYYADLIIDGKRLLKEKNKLGEGVVGGDLGPGTLACVSNEFVILRSTASNVLESNAYLKLEKQRRIIQRQIDRQRREANPDNFNENGTIKKGSKWIVSKRQKHNENQLADLQRRIKEMRRDNHGAIANELRSHGDEFRLDKDSYKGWQRSRYGSSLLKHAPGMLVELIRDKFENTGGKIDEINTYDSKSSQTCPNCGDISKKQLDERIHDCKCGFIMQRDIVSAIVNKFWNTEKKTLDVDKSKRFIKDKRSVLQAGIEKILQSVREGHLLPSTFGTIPELERIACQFDDQISSDACCLQKTT

SEQ ID NO: 7 - protein, 477 aa, unidentified
  REGION 1..477: note = uncultivated organism
  REGION 1..477: note = MG120-2 Effector
MKSKTSTYTVEFELITTFQDRYIINKKMKVAKSIYNACLNECLKRLKAVKADKEYHTLVKTESSNQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQISTVENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTNTLDVVDIDLCNLKFDNFVKLHDIEIQRLKNLNNKKLNWFVA

SEQ ID NO: 8 - protein, 477 aa, unidentified
  REGION 1..477: note = uncultivated organism
  REGION 1..477: note = MG120-3 Effector
MKSKTSTYTVEFELITTFQDRYIINKKMKVAKSIYNACLNECLKRLKAVKADKEYHTLVKTESSNQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQVSTAENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTNTLDVVDIDLCNLKFDNFVKLHDIEIQRLKNLNNKKLNWFVA

SEQ ID NO: 9 - protein, 477 aa, unidentified
  REGION 1..477: note = uncultivated organism
  REGION 1..477: note = MG120-4 Effector
MKSKTSTYTVEFELITTFQDRYIINKKMKVAKSIYNACLNECLKRLKAVKADKEYHTLVKTESSKQRNSRLKEIERYYGYSEYDLHSWVNQVKYHFEGHLGINEVQKLATRAFKAVEKFHYGKAKKVYFKSFKDDMSVENKSNKTGLRIKDLKIIWGNLSIPFKVKKNDKYAQLALLDKTKYVRVLKREIRGKERFFVQLIQEGLPPKKHQISTVENRVGIDIGTSTVAVVSDNEVFMNELATIKTPYKKLRKLDRAMDRSRRSTNPQNYNDNGTVKKGYKKWNYSNRYLKLKYKRKELFRKIASIRKQNHEQTANFILSLGLDVRVETMRFQSLQKRAKKTTRNKKNGKINKKKRFGKSIANYAPAMLLEIINRKLKYQNLDLKKIDTYSVKASQLDHTTGECTKKFLSNRWNNIDDKLIQRDLYSAFLIQNTTKTLDVVDIDLCNLKFDNFVKLHDIEIQRLKNLNNKKLNWFVA

SEQ ID NO: 10 - protein, 475 aa, unidentified
  REGION 1..475: note = uncultivated organism
  REGION 1..475: note = MG120-5 Effector
MVNKTSTYTVEFELITTFQHKYIINKKMKVAKSIYNACLNECLKRLKAVKANKEYHALVKTQSSKERNQCLKEIELSYKYSEYDLHSWVTQIKQYFEGHLGINEVQKLATRAFNTVEKLHYGKAKNIYFKSFTDNMSVENKSNKTGLRIKNSKIIWGKLSIPFKIKKNDKYAQLALLDKTKYVRVLKREIRGKERFFVQLIQEGLPPKKHQTSTVENRIGMDIGTSTISIVSDNKVSIKELANIKTPYKKIRRINRAMARSKYFANPQNFNDNGKKTLKKWNYSNKYLKLKSKRKELFRKLSIIRKQTHEETANEILSLGLDIRVETMHFHSLQRRAKKITYNQNNGKINKKKRFGKSISNYAPSMLLTIINRKLKYQNCELKQIDTYSVKASQFNHTTGEFTKKLLSDRWKNIDNKLIQRDLYSAFLIQNTTDDLKTIDIDLCNLKFDNFVKLHDIEIQNIKKSQNKLLHWFVA

SEQ ID NO: 11 - protein, 517 aa, unidentified
  REGION 1..517: note = uncultivated organism
  REGION 1..517: note = MG120-6 Effector
MIRKGHLVRLQTPSFVVSVKLLLPEQVQNHLEKSFHIANSAYNEALSFGLRRFDAMKQNSSYQELLELRRELLAKSDKEQKTKEFKAQKKENDNKLAEIRKDYSLTEFGLSQHLSQQRRKPDSAYQHLNSGELQVIASHVMKALEKVLFYQIEPHKVRFRSKYDADMSFRNRVNSTGTRLVESNKTSIAYRLYIHKASTFVDIPIKAFNTYQQMSLLRSERIKYVQIIRKTIRGKKVYYLQIVCEGTPPAKVNRGQGVVGIDPGISTVAYAAPNKVALVDLVPRNITQKEKFLKALDRRIERSRRVNNPTCFNEDGTIKKGSHFKHLSNRALRLVNRRKKSYRSLSEERKKLQGQLINQLVSNASVIKMEELNVKGLQKRAKDLQINPKTNRPFSKKRFGKSIFRAAPSAFREALKTRAVQLGIEIIMVSPKNVKPSQYNHILQTFEKKPLSTRIFDLSPEWTGLQRDLYSAFLIGHIENNQYQQTELEQDFPTFYQQMRDFLQQTPQTSRLAWYFS

SEQ ID NO: 12 - protein, 528 aa, unidentified
  REGION 1..528: note = uncultivated organism
  REGION 1..528: note = MG120-7 Effector
MPPCTSTSRSKSTSSTPSFVCELLLRVSPAQEKALLARLEAARQLYNACLGEALKRVGLEQQSKLWQKAWKAGDKELRRALIRKACLKYGFTDAALQHYAVKIRRSCWIGDHLDVHVAQKLGTRAFRAAQRVLFGQARKVRFKGKNQMDTVEGKSNGAGIRWRNGRMLWRGLVLPAIIDQEDPVIRHALSCRVKYVRLVRRKINGRNRFYTQLICEGTPYQKPENALAEGTVGIDAGPSIIARVNSKEARLDRFCEELAKKEAEIRRLQRRLDRSRRANNPENYNPDGTVRKGPKQWKKTNTYLKTEARLAELWRRLAAHRRSLIGKMVNETLRMGNVFKFEKLSYRALQRMFGKSVNIRAPGMFFQELARKAESAGGEVLEFPTRLKVKGTETCVGLSSYCVCGEHRKKPLSERWHICSCGVRCQRDLYSAYLACFVKQIGQNDFELDAGTAARQWPGAEPLLQAASGRIPNPQMSGSSGQPRTGTEGVAAEGGTAKTEARDVVAPPPGGGESPGEVAVVPLRTPGL

SEQ ID NO: 13 - protein, 425 aa, unidentified
  REGION 1..425: note = uncultivated organism
  REGION 1..425: note = MG120-8 Effector
MAKKENSKSFILTFETGLTKRDRDLCEKKLRIGRSMYNAFLGEALKRYNGYQKDDRYLTAVKLYRDAKEHNDKEEMKALSEVMNQVKSEYRWGGKYCLNDMRKEQSRHFKGAIGSMIAVSLSHRAYAAVEKMEKNTDVTKVSFVRQSDDFSIEGSTNDNGIIYRNGRIYFDISKDVWVDIKPARKNDDWYYDEALKSRVKFCRLLSRTIRGKKRYYVQLCMEGTPPNRREYGDKRIEILDVKISHAELKKEGEKPQIIELAPGCKNPEEKIAELDRKMDASRRATNPDNYNEDGTTKNGPLKWSYSNNYKKLRAKRAEIYRSMKERRKVSHEELTNRILAEGTDITIKHLSYKEQQRRSKKDTEINPDTGRPYSKARQGKNIANRAPSEFVSILSRKLNYIDKAFADESKENRKDEKNENKIIRI

SEQ ID NO: 14 - protein, 670 aa, unidentified
  REGION 1..670: note = uncultivated organism
  REGION 1..670: note = MG120-9 Effector
MPKPEGGTHVCEIPLQRSAHKEKILGVRFGCGYQAFNAALDEALRRIKLMRQSKDWQKARKLPKGNSDGSKEQKAQAKARGEAFNTACLKFGFTEFSLLEIWTSKPMSPESRLQGALKRKQRRLRELLARQSEIEALPETDKDRTKLLKENEQARSKAVSDLTLLAITPLESKPVGKLGGSWLVKHLGVHVCQAATSKAWVAIQEYSFGKKGKPKFRSKYDPPMSVDGKSGASLRWNKGRVEWGGLILEPLIKRDQHGRPVDPVIAYSLHKKIKYVRIVRKASLRGYDYAVQLVCDGPSYNYQREVGTATVGLDLGPSGIAIVAHEGSTVVKAELCKLAPEIIRNSEKIRRLQRKLDRQLRANNLDCYRPNGTFIKGSKAHNRSNRQLGTKLQLSLIQTKQAAHRKSLHGRRVNELVSLGVNATTEKLNYKVWQGHWGRSIRDRAPGLLIEMYRKKCERYGGKLGECSTKTTKMSRTCHCRTVKFKTLSQRWHSCTCGVGPVQRDIYSAFLASCVKQDVVRGDQLDVESLKKLWDNGADKLLKAASSEAVKAGLVRPYRKCEKLTPHGVDSPSNGSEHSSEDPDSKLEGISPEHVAEDEIQDVVPTACGKGEPGGVFEPNRNATDLSTHRERRGGETRQPQSSQQLRLFEGDSKQSLSQSTTQVQEIVEL

SEQ ID NO: 15 - protein, 786 aa, unidentified
  REGION 1..786: note = uncultivated organism
  REGION 1..786: note = MG118-1 Effector
MKQKDDFTIIDRLLLSNFPLTSYYPAKAKDKREALRVFERCGEQASIDYLKTRIGAKSPIKQLASKYLPNAKFLNSTLKVAARKLKEQGEQKALEYLQANVSEDSYDLKSPVKCEIVATSKKLEDFAIYNASRDIQCYIYNLSKDEFLSCVPGTSKESHQKWFSAHSALKQYETLGVQCLNLIFSNAISTYKGVEKKVAEANAKKIKKIEERKARGLVVEEFEPKKVYGEDNKLLEVPGINPVIYCYQQISPKVYVSGQVDWAANSSELQIYRGDIEGYKQYDRKPDDPIVTLVTQKSRLEISDGSPGHIPEKDKDTLTLNKHRRLHKYWSSEVEKSKNAILVVIRMFDGWVLVDLRGLLRQCRYRKLIAPNQKITVKELLSMVTGDPVVDVERKVCRFIFKPEIIRVKKKNTIKASKKVAENLAKQQTTLLSIDLGVTNVVAASVYSVVDGKMERLMEPFALTDEQKDNFTRCDEKRDRINANIRKEAIAFLTLEEQAEIRKLDTFGPEEAKIAICKKLGIDDVSSVPWDSMGANTTILSDIYLANGGDLEKVYFTPKPKKNGKGKKVPDPIKSKDFYWSRKYRPRLSTETREHLNEKIWELQRANKDYQKLAIRKKELSRQVVNYILAKVENRTQCGKVIIALEDIDVKMFHRKDARISGWDGFFVAKSSKQWLIKEMHKAFSDLSQNRGYIVIEVRPAYTSQTCPKCGYCHRDNRDKNNREKFSCKKCGFTAHADFDVATYNIAKVAVTGERLKGPEKKNKKISSKEVSSGYMLQELPSLLEI

SEQ ID NO: 16 - protein, 640 aa, unidentified
  REGION 1..640: note = uncultivated organism
  REGION 1..640: note = MG90-3 Effector
MSFGPSKIVFKMGAKLPLDDGPAVVDRIISGAHKYYNKLIEFGRRQLLEKEELRREFLPGLAEAADRIEEARQAHEALADDIRRRNQEARRRQATPEDLALRASSANRLAEARRTRKEILARVKDDLRYLQIQEQRHLRDNRTLAALYNEADCNWGTRLQTGRAFESAMKIMNPATNKMMERGVDNPPKFKRWTGDGLVTVQTQNGLSLEDLLAGKNGQFRLEKLPPKNGNLHRALAWLRVGSDGDGEAIWLKVPVQWREDFDRLPPGSMIKQASLVRRRGPHRRFASPDNVLGVWRPYDRFELQMTIETSGIRDTSKLATAGLCGVDLGWRLKEDGSLRVAYWVGDDGQEGEVLVPADRWLICNELEIICKDNFNQHVAILSRWLGAQASLPETLTEKTSTLLSWKSIGRLCRLVDTWQPVPGDEDLIKVLRAWRVQEAHLLDYERGRARMMANWRKDHYRQLAAFLAKQYRTISVEDCDWRKLNKLPPVESNDPVNLTARRNIRIAAVGQLRDCLRQCGANTILSETENTTKECHVCHSIEEFDHRKLFHTCSQCGNTWDQDRNAALNLIARAEKALREMAEIETETVPVEGVGAASSVLGSRGSQDQASEEVVAVPPKKGGRWQRRKAKSSRRAAAT

SEQ ID NO: 17 - protein, 611 aa, unidentified
  REGION 1..611: note = uncultivated organism
  REGION 1..611: note = MG90-5 Effector
MPFNNKASESGLDTRVYAYGCNLKKDVADVLMIQLRLANKYYNRLVDCKKDQLHAERETLYKLSPALKQLAQEIEKKDDEKGELVRQAKRMNVAARASGAADDPLRAELAGVEAELRDLYDREKALAKPLYRQPEFVDERARLQAVFEDACKQVRKESRTPNEHGDVLGWGTAGAWYDAFKAACKAPMVLFKRAKKSGDTRLYVAACKLLMRGPKYRRFSGEGRVGVQITGGGVPVPDVLDGKSTFVQIDPADPSTSSRPKRRNGRIRIGSGSTTRVPLWADFRFRMHKGHALPDDGIVKRAYLIAKKIGPRTKWEFQVSVAGRPKDWPDRNVTDGVVAVDVGWRDMGSHVRVACWLGDDGRYGEITIPAERVVRYQKVKDLQSIRDKNFDAIQAALMAWKKAAKDDVPEWFSERFESLHSWSSQARLVSATRYWEQNRFDGDESMFEEVRVWWRQDTHLYDWQESQRKGEERWRRDHYRKIAAELSRRYRKAVVEDVDWGVFQRAQDPLKAKEINEERRAAREERRRIAGVSYLIGDLKNRLVEVFEAPPEWTTQECHHCGCIDHFDAENQLRHTCSGCGEEWDQDYNACYNLLRLEREAASGSGVEDGQ

SEQ ID NO: 18 - protein, 762 aa, unidentified
  REGION 1..762: note = uncultivated organism
  REGION 1..762: note = MG90-6 Effector
MKLKTSSIPTQIWSFGTPPPKFWSPGPSTSEERPRYRRLGEPPIPLAQFTGEPFVREVLFRGNRFYNDRIALERTRYAEYVRIRRSFSPALAACEDAYVAADVAAQKAVQDIKDARAKQYRQSKKKSDEVPAALIEALRVARAQQKETKTQLKRARAHFEAQMKPTRLEYDTRCQTRKLALASKSGITSPHYKGRVNREVLREMVAETQWPQAWKDLQSMEAAKVRQQKVVRKQRPVGAGIGGLIEKATNQAVKMSKFGPPGFSRFECDGRVGGQINPPCSMRDLFTGKNSKIRLRPQAPKLKRKHNLNHGKWKFDNQFFILALNIGTEVRNQAKWIEVPVRIHRPLPPDGKITQAWLLVRRQGPKLRYSVQLSIESESFLLPKKMNNGRERPIGVGRVAVNFGWRMVPGGVRAAYWTSETGERDELVLGSAEQEIFYRLTHPDVLRKHADEHFTEVRRGVRRSLRSGGGARLSKTPTVAETGNRLTHWDRWARKDEDSQRRQFTSLLWQWAKFRIGKARLDALWHEWKGYRLAHTEARPDGLDLFAHRNVASEWAKEHGLTKASERLAWWCHLWVRKEIHLAKYEADLRFGSHNQRDAIYRTLALKLSTQFGELITDNTKFSDLARREKVGDEGTEAWEKARQNRQAVGAGRFRAIFRDVFGQRGIVVSAVDNSVICSRCRQKMNVVAGESEVQCKQCGPIDVDHNNCLNMLRRADDGERITRDQKEDAPPEQPSTPGPENEQSTGPADSYTHDSLWDAAE

SEQ ID NO: 19 - protein, 585 aa, unidentified
  REGION 1..585: note = uncultivated organism
  REGION 1..585: note = MG90-7 Effector
MSGIRILNWCYGAYSPEDRAPVFEQLRLAHRFRNRLCELEINCRNEIEAILCERSEEYVSACRNVSDCEGQIEEIYKAVKLRGQQERKKPKVSDDEAEAIKVLKSQRLKLWELRRKARTAAFARSDVEYSISAIRNRHNDLRKQARVADGLYWGTAATVIDSAKSFSSGAPPRFTAYRGQGTLSVQFINGMPAERLLSGEDRRARLIWAERNRYLLWMRIGSQDDRSPTWAKIPVVMHRPFPTDAILKWLHLDLRTCASDEHWTARFALQTASTVRFPIRPLVAVHCGWRTLDDGSVRVAVWRDTEGHTGDIRLTNKDRLSLEIPEGLQSVRDKHFNEAISFLKQQFKSADIVPEWFSVRTTTMLHWKKQSRLAGLVCYWRDHRFCGDEIAFDTLESWRKRDKHLWLWQQNQRRKWRIRRNQLYRSFAKDLAAKYGRVSVMAAEWAKMRKVPEVEEVERQSTRLRRLSAGAAVATLEGYIKEAFGRDCVVAIDIKNVSRQCASCGQLQTEFTKRVGDCDKCGTRRDIDDNGARNQIARAGLTLKSLEPLADVMRQRLTAATSKPAPSERHQRRLAARKRLAEAKQ

SEQ ID NO: 20 - protein, 589 aa, unidentified
  REGION 1..589: note = uncultivated organism
  REGION 1..589: note = MG90-8 Effector
MFGHTSAPSRIFRFQARLPTAGLEVIEEQLTLAHRYRNALVALERRRRAMVHHALVQAMPTGVAWEATVAMLEAMLESHRDVLARQHAKKRRREISAEAKAIAAILKQALREARGHHRALRRLAAGKGLDADDHTHQMLHHCQLTAQDASEALHQVDEAIHAERKRLRANCGLYWGNYLAVEQGMQAARSGPPPRFRRYDRQGKLAVQIQHGMSPEELHSLQDPRLQLEAPKVDDLGRIYRRHRRVLCRFRIGSVGREPLWAELPVIMTRPLPYDSRVKWAYLKRERVATHLYWYLELVLSRDKGWPKPDAAQTGIVGIDPGYRRRPHGLRVMYWLGDDHQGSELVLPEEWLQKWSWVFELAALRKRRMNAIVADLVRWVKDHGRPAWWPEDTRTLARWCSPAQLAALTLQWRREHADEAPDMLARLEAWRKMDKRQYEEQEHLRRHLIDARDDLYRKTALALRRRYQTAVIEDTDWEALARREEAEEVRRLPAIVGRYRQIAAPSRLTQILREAFAEVLDAPSGYSTQRCSICGRLMGITRRDGTYVPPDNASLEIRCPWDHTLHDQDANAARNFRDAGPALRKRAAR

SEQ ID NO: 21 - protein, 625 aa, unidentified
  REGION 1..625: note = uncultivated organism
  REGION 1..625: note = MG90-16 Effector
MVQRSTFTGHNCVYVYGTPSKVDIPDYAMEQLRLANRFWNGLVEIQHAHEQAVAELWASHPELAKLNDEIAQLVEWGRELKEKADAARVKGRRRYITDADLRNELKQQREQLIAAKKSYRESKSLFYNQLKPQFTTLKEKRVADIKALRQAFASQGLYWGTYNAVSQSFDTAAKRVSSLRASGRSSALRFHRFDGSGTWTVQLQQEAGDPPRTWAALVGGQSKWKNDVRIAPEELYEGGMRKHPRTLLSIRCGRQGKDALYLTIPITVHRPIPPEAEVTQIQVSRKRVGTHYELSAAFSLRMPTPAKTKNPGVVGINFGWRSKRSDELLRTAVWYGIGRDTQLELREWMTEWVTQERLAKGKMVGEIRVPDRWRRVMARMHEIRSQRDLNLNAIRGEVVKWLTTHKRSANQLASAWAQAHPNASKPLWNKPSDVGLWRSAGRLAALCLFWRTHRQSGDETIFAVFEAWRKQDKHLYDWEAYNTRQLIVQRREVYRCVAEQLTSIYGHVVVDDANFQTLTMKETRLEEADSEQADLARSQARDASPGLFREIVKHTASRLGTVLTVEPANGITQVHSVCNTWVDADAAQKIMLWCPVCETSFDQDANAAMNLVRLATGQVAQLVKS

SEQ ID NO: 22 - protein, 625 aa, unidentified
  REGION 1..625: note = uncultivated organism
  REGION 1..625: note = MG90-17 Effector
MVQRSTFTGHNCVYVYGTPSKVDIPDYAMEQLRLANRFWNGLVEIQHAHEQAVAELWASHPELAKLNDEIAQLVEWGRELKEKADAARVKGRRRYITDADLRNELKQQREQLIAAKKSYRESKSLFYNQLKPQFTTLKEKRVADIKALRQAFASQGLYWGTYNAVSQSFDVAAKRVSSLRSSGRSSALRFHRFEGSGTWTVQLQQEAGDPPRTWAALVGGQSKWKNDVRIAPEELDEGGMRKHPRTLLSIRCGRQGKDALYLTIPITVHRPIPPEAEVTQIQVSRKRVGTHYELSAAFSLRMPTPAKTKNPGVVGINFGWRSKRSDELLRTAVWYGIGRDTQLELREWMTEWVTQERLAKGKMVGEIRVPDRWRRVMARMHEIRSQRDLNLNAIRGEVVKWLTTHKRSANQLASAWAQAHPNASKPLWNKPSDVGLWRSAGRLAALCLFWRTHRQSGDETIFAVFEAWRKQDKHLYDWEAYNTRQLIVQRREVYRCVAEQLTSIYGHVVVDDANFQTLTMKETRLEEADSEQADLARSQARDASPGLFREIVKHTASRLGTVLTVEPANGITQVHSVCNTWVDADAAQKIMLWCPVCETSFDQDANAAMNLVRLATGQVAQLVKS

SEQ ID NO: 23 - protein, 640 aa, unidentified; identical to SEQ ID NO: 16
  REGION 1..640: note = uncultivated organism
  REGION 1..640: note = MG90-18 Effector

SEQ ID NO: 24 - protein, 634 aa, unidentified
  REGION 1..634: note = uncultivated organism
  REGION 1..634: note = MG90-19 Effector
MAFGNKSKPTRKYNYGCRGFLSGPREINPDGTKALRQRFDGHDLAVTQMKLAHQFQNKLVEIERARREAIDPILVRHFPALTTLHNAAQEQQERLQELRDELSQSNSQNRQLMSDPELVEQINAQKRVVSQAWEFYSDQRDAAFADPKVKADLKDNDKSFEQRKADARNAAVEGGLYWATSLQVVGRVKRTGPPPKFKSWRGEEVISVQFQRKPDKTSPKEPVLDSKGNPKIHPRSKKPTFAHVGGSSLRTCDVFTPNTNCWIERTYQPPLTAPHPKYVVIHFRVSSDEKGKPVMASIPAVMSRPLPEDGEVKWVHLSRRKIGTHYRWDVQFDIARDAWTYHPAGQDRAQEGTVAVALGWRLIDGEIRVTEWVGDDGVTGTVRIPKELVEGWSYLDTLQSIRDTLFEAERAELVDWFVNYPNPLPEEWTERAQTLIQWRSADRFMWLIWWWKDHRIPGDEEIFQRMWGRIQLNPTTGRNQYTGGRLQSKHLCDWRAHKRDKIKNWRKDFFRKVAIDLSYQYKDVVIAEIDWNKLAENPEVENGNDIVNKRYRALSSCAQLRDEITRYMNEVTESARNIVTTCCQCGESCDHPKSGRWIRCESCGGEKRDRAVNAATNLLNRALGASGAKVPARV

SEQ ID NO: 25 - protein, 643 aa, unidentified
  REGION 1..643: note = uncultivated organism
  REGION 1..643: note = MG90-20 Effector
METRILEFRAALPLHRYPLHKAGLLGADLAERNSEKPCFLEACYEYRRQLVNIELYRRQARRAVLLKLYPRLVALSDQIKAMTVELTRLRGQLSSARARVRARITLPAVEKQIKLQRAALAKLRTEYNKAHERATALSSLEGAQASLKSNRNKNKPAKQETLHESIAKYQEIADSYTPSELDSSRKLADEYYAIAKDVDARKSALYGSSDLHWGTKNAISEGVEASRSGPPPQQRRGEGEVMFSVQLQAARRGGETRSANWQALLTDNAWSSQVRLRAIPAAEVAGFSKSRPGQAIRNGVKSQHQHMLQIRVTADSEKGWVEMPFHFRPDRWPADAKILYVRIRRRKLRARYQWLVQFVVTSEQFSRQVGTGEVGINLGSRLLKNGLRAAYAVDDTGHSETLELPHEDFVRHWRYTEELQAIRDREFTAMLTSLRDWLKRHKKLLPGWLREEAKTLHLQKAKRVARLVMRWRDNRFDGDEKIFADVNYWRSRKNGHLLEWQNSEMGRTNGRRKSLYQKWAADLRKRYGTVYIGKINYRELREVEEPEDEAGSSEVRKLAARAAIGSLKVILQMSGMKVVERDPSYTTQLCHVCGHVNDFDAASRLEVVCESCASCYDQDYNAAHNLLTGRRPKSAGKELQAVV

SEQ ID NO: 26 - protein, 705 aa, unidentified
  REGION 1..705: note = uncultivated organism
  REGION 1..705: note = MG90-21 Effector
MMGAQVRRGGVVTTQSKTKKRGGRKKRSKEELREAQRARMRKRNKAKKKAKEKDKEKGIKYKDTGVRVYKYPANRTLPLGFENEHRFLGQLAEQGDRVNKDLISAEMRRRAHEEGLLLEGVEFVDDHFFHSQRMRNDTVEIDHSMRDLDMKFMLKTELKDEILEADAKYAAIKKALHAVHEEQRKRNSKNRDDTLTPEERAEISALHKKLKNAWEDRQKICKEARELDSYDKKAFDDAKSDAAKEGRERFPELHWNTRNTRHEALRQHRKGPPPKFKSWDGSGIIGGQTYQSREKQAPEVIFSGDLGQGHARIVRYPTGVWVAGRDRAAKRGDAILYLRVRSEGKKPVWARIPFFMKREIPEGVRITHAKVVRKKEGRRLKWSVQLSLADVGPRPDRAKEGTVAIDVGWRYDNGSIVVATFSGSDGREGKVALPCWWLREAKQVENLQSLVKKDFNEAVDNLIAYFKVEGRGCIPKGLRKEFKNLHQFRSPKKLSKLVKKWRDQDEDSCLDWLWHWRGRQLHLGPDWRDPLRGQLQGSRRDLYRRFARDMGRRYKNCVIEEMNLAHMQERNKAGDNKKQDRINKNKRHAALSVLKKSLQNVFFEDIDTLNPAYTSQTCWSCGKRNRSLGSAQVFTCSCGETWNRDVNASRNLLAASGAKVNWDRPALEPDEVWTYSEKPNKRRRKTIAKKRALEMKRKNEGGEEA

SEQ ID NO: 27 - protein, 698 aa, unidentified
  REGION 1..698: note = uncultivated organism
  REGION 1..698: note = MG90-22 Effector
MEPVKKSNARKTSTSETKVYSYGAFLPKESEQIKLINDQLYFAHKYRNKLVEIERKRRNRFRNLRKLMSPELRQLENDLLLTEEKIIELRKSFGGRAENSLDPKFPKKNRQLTPQAVEIKDLKLKKKDLSAKIKSIHTQLNLDYFKEADSKFSLLKKERLEQKAKELNKDSLGPNDVNRHNVVNNLYKEMIDGGNRFWAIKAKISKSAEASNKRARSNCKCSSGTYVSIEEAAKQSFSNSKFEPKFKSFDGSGKIGMQLTQNKGLSIKDALSGSSPVLKIDLHPEVYLRQNKKKNKVLATARIKLFGTEKTGKFVDIPFIMHRQMPEDATIKWVFLVVSKIGYRSIYNIQFTIESNSFTQPSAIRPDDVAINLGWKVNDQTDDITVATSFDGKNYNELILPSKMRANIVYKETLISHADKHFDSVKKDVSKWLKNSNLDECITKYFTNLPQWRSHKKLLFVSKELAKVFLPDNTWYDLWKKWKTHCKENKPWKNCSDKDDLFTTLDNTIQWCKDNSIDDPNVQMAFYLKTWAEKEIHLINWARGIESKLRKHRKEIYRCFAKKLSSTYGKVIVENWDKSKTAETPDVENDNRTKQEENANAVRQFVGVSVLTDALKQKFGKDFCEENAKNISKEHFKCGGELINQKELSDVHCKKCNKSVNVNYNAAAHLFDRHGERSGAVKLPGTARKTRKSPELLA

SEQ ID NO: 28 - protein, 685 aa, unidentified
  REGION 1..685: note = uncultivated organism
  REGION 1..685: note = MG90-23 Effector
MLVYKWGIGPLPLEARQIIDREVRAAHRYRNRLVEIERSRRAAYRDLRNSLSPELAHLHAAYAAADRAVVEGRRLLSGVPRAERARHPVAAEVRRLSAARSGAWKAYAEARDRTTADVFGAADQKYREAKQSVTAAVGFACAVARFARGADPGPIGPHVLAAITAAVRERALADPAVGEPWKAKTRSQMEHEALAKRARAECGCAVGTYLAVEAAAEKSFAECAGDPPFSRLECERVGLQVRGGGLSADDVVGAAVGQVRVEFPADMTARANGTRYAVVHLHLSGRGDQATWLHLPVVWHRDMAPEARVRWAYVVARRVGLVWHYELQLTCDSVDRARAQSAGARGTIALNLGWRALKNGDLRVATPWSGSASRAEDRLVLPRSFREGSDLADRLLSYADEHFLAVRDALAGWFKGGERSLFSPEQVAAWSLDTVHAWRSHGRLARVALDLRRDWLEARGVDVPALWKAWRLERSPKLDLFGPLDEIKAWLAGRGVVSADQVLAVYLDWWRAKDRHLVNWARNNDLRLRRSRRDRYRCYARDLAARYERVVIEQWNKSETAETPDPEADTRTEQEVRGNSNRVLACVSELVDALEAAFGEANVHRAPSERITVEHHGCGGESSDPLPQIPVTCFACGQVYDQDLNAAKHLYDRHSGEPSGGVNVGGGARGAKKSRRVEGFGRAAE

SEQ ID NO: 29 - protein, 680 aa, unidentified
  REGION 1..680: note = uncultivated organism
  REGION 1..680: note = MG90-24 Effector
MTTTKLYSYGAHLAHCPAKVGALLRKSVEYYNALVALENLRRAMFLGIEEDASSDVAEAMKVVNEADAAVELAFGAISEHRKTTRARSIPVDLRSAKEAAIERKKAAKITLREAKAKHRAQFDKPNDEFLRRKTAATSDASLSELRELSGEDLRKKIASIREKLGPIAPRTAEKLNAEILGTMVGESEWSEHWKVVQFLERVIADHEKEARRISGLPHGTYSLVDDAFEAAKRTQKVGDLRRKWIQREGDLPVDDSGRVGVQLPSGLSIADLCAGNHARFKLCVETPEERAVKGLGDRSARWERRNIRAIATIAIDSSDSGWEYLDIPISYHRSLPPNATVKWVWLFVQRHGMRLHYDLQITLVIPDDVDLGQSRRAEVCTINPGWRHFDDGSIRVAYVVGSDGHWEDLRLPATEGKRWSVSEQMKFSDSLRSASDSLFNEARKVLVGMLPRLPESIQLQCTFIAQWKDHTKLQRAVIATLAESWTSNSCQNAFEKWVAQRKARGLDLMPTLDEAVEFSPTDPERWYLYCWLRKDRHLYQMAIDISRKAARDRNQMFSIWAKRLSERYQRVALAEGGLKKLAKKTDEVASSQLRAKSRVATGTFVAKVKEKFGKPNVIADKGVNASRTCVKCGHLHEEIDLSRDHMYTCSQCGYEQDQDETNCRNRLTWLRDRRGAEKTE

SEQ ID NO: 30 - protein, 433 aa, unidentified
  REGION 1..433: note = uncultivated organism
  REGION 1..433: note = MG119-1 Effector
MVYTTKFYLIYDDDNNPIDYAAFCKEMFGIQRDIRTFKNLASSEYFSYMMRRTRWKDEHGAYPTDEELLGDKIGNILYRLACDEIPSFYRINANCVSRDVMKYFKTRWKDIISGKMSIPAYKSNQPINIHNKYVVPIDVDGDIGITLSLLSSDRKKALDLETGMFRFKVWHKCESSIPILHRCLTGEYKIGESSLQYDKSKRMWELALSYHFEKREHELDPERILGIDLGICIPVVVAVSDSYKRLFVRGNEVTAFREKTEAMRRQMSRARVFAGEGSVGHGRNIRAKPVDRIGHRIANFHNTKNDAWSREIVRFAVKNQCGTIQMEDLSGISSGDAPRFLKNWTYFDLQQKIKYKAEAEGIRVVMVAPKFTSQRCSRCGYISSENRPTQEQFVCQSCGYTANADYNAARNLAIKNIDQIIKKECEPKANRKS

SEQ ID NO: 31 - protein, 493 aa, unidentified
  REGION 1..493: note = uncultivated organism
  REGION 1..493: note = MG119-2 Effector
MGKTDIKEEKIFIGSKKVKITIVEDDPDERNRKYKLIHDEMYKQYLALNMCMSLLATQNTLRMYNSGAENRLLSQIAKLDKKISEKQQIADKDANTEKEQTRKQKAIEDIVKLEEEKNKLREEFDKKATNRTDIDVKFHEQYTSDLYQIIRNEFDFASSITAGLVARKAKDDFKNSFSMGYAKGERSLISYKRSFPLMIRGNEDACYDSNKRKWLDFYYDGDDILIRWLDGIIFKAILGNNPNRNMEIKHTLHKIITGEYRVSQSSIEFDDKGHMYMNVSYRFKKNSKTEIIPGRTLGVDLGIATPAYVCLSDTTYVRSGIGSANDFFRIRQQMKSRRRSLYKSLSLVKGGKGRKKKLQAINSLRDKERNFAKTYNHQLSNKIVKFAKDNGCEFINLEHLTKNGFEDAILSSWSYYELQNMIEYKAEREGIKVRYVDPAYTSQTCSKCGHVDKENRLTQAKFKCVNCGLELNADHNASINIARSDNFVDKKKK

SEQ ID NO: 32 - protein, 486 aa, unidentified
  REGION 1..486: note = uncultivated organism
  REGION 1..486: note = MG119-3 Effector
MATEYICITRKIEVHLHKHGGSEEAVQRLKDEYQIWDTINDNLYKAANRIISHCFFNDAYEYRLKIHSPRFKQIENLLRYSKKNKLSEDDIKELKAERKLLYSDFKKQRLDFLQGYGGKGSEQNSTYQVVTHEFLDVIPSDILTCLNQNIAGVYKQYSLDVEMGRRTIPNFKKGIPVPFAIKKHQQLRLRKRDDGSIYVLLPKGLEWDLSFGRDKSNNREIVERVLSGQYDVGNSSIQESKNGKRFLLLVVKIPKESRVLNKERVVGVDLGIATPLYAALNDNEYVGLSIGSRDQFLKVRMRMAAQKRELQRNLCHTTNGGHGRTQKLQALERLEGKERNWVHLQNHIFSKSIIEYALKNDAGVIQMERLTGFGHDKNDEVDVEYKFILRYWSFFELQSMIEYKAKAADIEVRYINPYHTSQTCSFCGHYEKGQRINQSTFICKNPDCTKGKGKQKSDGSYEGINADWNAARNIALSNKIVDRKKK

SEQ ID NO: 33 - protein, 490 aa, unidentified
  REGION 1..490: note = uncultivated organism
  REGION 1..490: note = MG119-4 Effector
MPIITRKIELKIVKDGLTDEEYDQQWKYLYQINNTIYLAANRISTHCLFNDEYEMRLRLPYKSRYDKINKGLENIKTELEKLNTKKKTSDKEKRDRLLNKQRQLVDERNELDEDVKNKKKDFFQCSKQNSTYQLASKEFLKYIPAEILTDLNRYVQNNHNNNKNKVKSGERALSTYKKGMGIPFSIKPESGLRLFVKEEGIYLKWFKGILFRLEFGKDTSNNRCIVERLIESDKQQKKNKGEDYVANNSSIKLIKKGNDKSTRIFLLLSIDIPAKKQVLDKGVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 34 - protein, 462 aa, unidentified
  REGION 1..462: note = uncultivated organism
  REGION 1..462: note = MG119-5 Effector
MTTYTAKIQIVKPLDTDWNTLGKILNELQYETWKFSNKAMQMLWEFDGIGYEYFNKYKKYLNLKEYPFIKDDGKKITTIDSHINNQLKELVKHMNSAGVQATIKNVIDKWKADKKDVQYGRKSVANYKRDLPIELHNKQMFTHDKKPNITIDSRGKYIIEIKLISQKYAMELGRKGTETGFKLLLAVKGNYLKSIVDRVISGEYKLSMSKIVRDKRKKKWFLNLAYTFTPKKKQLNPNRIMGIDMGVNIPAMLAISDSPYYRQAVGSKEEVESFRRQIEARRRSIQRQRKWCGEGSIGHGVKTRLKPLEKLSGKIARFKDTKNHCWSRYIINEAIKNNCGTIQMEDLTGIAKDKAFLKNWTYDDLRQKITYKAEEAGIKVVIIDPYKTSSRCHKCGHIHSSKNKDDWRPKQEKFICESCGFKTNADWNAAKNIATKGIEKIIKKQMEKQDQEYKHGLKYVVN

SEQ ID NO: 35 - protein, 457 aa, unidentified
  REGION 1..457: note = uncultivated organism
  REGION 1..457: note = MG119-6 Effector
MAKASSIITRKIELIPDIEGMTQKESNDRCYGLIRGYDSNLYKVANLLVSQLYGVESLFQMLCLQNNEYMDCKRKLCNKSLSDDERRKVEERFEAVKEAITKRVRELAPQPAQTFAYRAATNSEYSKFFPSDILNSLKQDIYKHYNASKRDQQMGERSLATYKKGMPIPFMLEKHNVIKEIDEDYYLFWFEDTRFRLNFGRDRSNNRAIVYNCINTQKYKLNKMADIQIKNKKLFLIISVEIPKEQREAIKGKVMGVDLGVVHPAYAAVNEGPERSLVGNGEAFQKQRDVFRRRFRELQRCQLTQGGHGRKHKTKATERLRGKERNWVQTENHRISREVVNLAIKWKVETIQMEHLKGFGKNSDGDPSKEHKRILGRWSYFELQKDIEYKAAKVGINVKYVNPAYTSQTCHHCGERGDRSERDFFVCTNENCICYNNRIDADWNAAVNIAKSKDFIK

SEQ ID NO: 36 - protein, 315 aa, unidentified
  REGION 1..315: note = uncultivated organism
  REGION 1..315: note = MG119-7 Effector
MSIPFPWNHTIRIEVVEKENKEETEKRNRRDNEEYDFYLIWYNGIRFRLNFGKDRSNNYQIVKRCFKLDENCNDNYQLKTSSIKMVNGTKEMYLLLVVDIPKEKHDLNNKIVLGVDLGINTPAYVATNVTEDRKAIGSREHFLNSRMAFSRRFHSMQKLQGTANGKGRAKKLEPLERLRGKERNWVHTQNHLYSREVVNFAIKVKAATIQMEDLSGYGKNEEGTVKEEKKFLLGKWSYFELQTMIMDKALKAGITVNFIKPAYTSQACSWCGERGIRDGTTFYCQNPNCKQHEKKNINADHNAARNIARSTEIVK

SEQ ID NO: 37 - protein, 464 aa, unidentified
  REGION 1..464: note = uncultivated organism
  REGION 1..464: note = MG119-8 Effector
MIITRKIELYVHADSKDKKDLDWQMLRYLDNTIYKMANDVISHQFLNQNLSSRLLVHNKNYKPLKKEIDIIEENLNSELSKENKTKLLLNKKKLYSKLTILKKEVDERTKDILNTSIQNTTYQVLSDKYSLVHSQIRTCLNSIVQKNYSNDIYDVLSGKKSLATYKKGMPIPFQKPEFKKNKFDIYFTFPGKINFGLHFGKDKSNNKIIVDNIISGKYKSCDSSIQIKDKKIILLLAVDIPENKIKLDKKITVGVDLGINTPAYAALSNNVCGLAIGKRDEFLNVRLRLQKSRRDMQKNLSFTIGGRGRNKKLQALDRYSEKERNFVKTQNHIISRSIIDFAIENSAGVINLEDISGITKDNTFLNRNWSYFELQTNIEYKAKQNGIEVKYINPAYTSQICSFCGHHSSTQRDKERFTCENDNCTNKDKHGNNKTINADYNSARNIAKSTNFQEKGTNKSKKDL

SEQ ID NO: 38 - protein, 472 aa, unidentified
  REGION 1..472: note = uncultivated organism
  REGION 1..472: note = MG119-9 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRCGEKINADFNAARNIAMSKKIVKDN

SEQ ID NO: 39 - protein, 471 aa, unidentified
  REGION 1..471: note = uncultivated organism
  REGION 1..471: note = MG119-10 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKKKGEDYVANNSSIKLVKYGKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKGKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 40 - protein, 453 aa, unidentified
  REGION 1..453: note = uncultivated organism
  REGION 1..453: note = MG119-11 Effector
MSKITRKIEIIPDIEGLTHDESNKKCYGAFYSFDKNLYKVANLLVSQLYGLDNLLSLMRLQNDEYVKCQYKLSLKSTTDAEKENLKKRMEKIDAELVSIKNGMAPKHPQTFAYRAVTNCVYAKNIPSDILNTLKQDVYKHFNDTKKEQFRGERSLTTYKRGMPVPFSIEKKHAIVCDGDNYYLPWFEDTRFRLNFGRDKSNNRAIIDNCIKTKRYKLCAAAKMQFKDKKLFLLVTVDIPATETTTIKGKVMGVDLGVVNPAYVAVNDGPERSRIGNGEAFQKQRDVFRRRFRELQRSQLTQGGHGRKHKTKATETLRGKERNWVQTENHRISREIVNLASRWKVEYIQMESLKGYGKNQEGEVEDKHKRLLGRWSYFELQKDIEYKAAMVGIQVKYINPAYTSQTCHVCGQRGNRIERDTFICTNPECTCYNQAQDADMNAAINIAKSKDVVK

SEQ ID NO: 41 - protein, 483 aa, unidentified
  REGION 1..483: note = uncultivated organism
  REGION 1..483: note = MG119-12 Effector
MPVITRKIKLNLCTEGLSEDERKAQWKMLYRINDNLYRAANNISSKLYLDEHVSSMVRLKNAEYTSLISDLMKAKKAEDEAVITDLETKIESLKSEMTAQEEAICSYATEMATRTLAGKFASELDLDIYGQILAEVKSVVFKNFNSDSKEVREGKRSIRTYKKGMPIPFPWNKTIRLEAVKKELSGKHDEDEYDFYLNWYKSSRTDKKAIRFRLHFGKDKSNNLLIVKRCLHIDKTSSENYQLQTSSIQMKKGPEGAELYLLLVVNIPQEQHVLNKKIVVGVDLGINVPAYVATNCTEERKAIGDRDHFLNTRTAFSRRFHSFQRLKGTAGGKGRKKKLEPLEQLREKERNWVHTQNHHISRDVINFALQARAATIQMEDLSGYGKDEEGNVKPENKFLQSRWSYFELQSMIKYKAAKCGIKVNLIAPAYTSQTCSWCGQMGLRESTSFVCQNPECKQYGKDIHADYNAARNIARSNKIVKNE

SEQ ID NO: 42 - protein, 453 aa, unidentified
  REGION 1..453: note = uncultivated organism
  REGION 1..453: note = MG119-13 Effector
MSKITRKIEIVPDVEGLTHEESNKKCYNTFYNFDRKLYKVANLLVSQLYGLDNLLPLMRLQNEEYVDCQRKLSRKSITDAVKDEIKKRMEDIDAKLLSVKNEIAPMHAQSYSYRAVTSSECAKDIPSDILDSLKQDVYKHFNENKKEQIRGERSLTTYKKGMPIPFNLKKKHVIISVGDNYYLPWFEDTRFRLNFGRDRSNNRAIIDNCIKAKKYKLCPTAKIQLKDRKLFLLVTADIPETENVPVKGKVMGVDLGVVNPAYVAVNDGPERSRIGNGDAFQKQRDVFRRRFRELQRSQLTQGGHGRKHKTKAIEVLRGKERNWVQTENHRISREIVNLAVRWKVETIQMESLKGFGKNQEGEVENNHKRLLGRWSYFELQKDIEYKAAMAGVAVQYVNPAYTSQTCHVCGQRGNRIERDTFICTNPKCTCYNQAQDADMNAAINIAKSTDVVK

SEQ ID NO: 43 - protein, 486 aa, unidentified; identical to SEQ ID NO: 32
  REGION 1..486: note = uncultivated organism
  REGION 1..486: note = MG119-14 Effector

SEQ ID NO: 44 - protein, 469 aa, unidentified
  REGION 1..469: note = uncultivated organism
  REGION 1..469: note = MG119-15 Effector
MPTITRKIELHLCTEELSDEQQKAQRLLLYHINDNLYKAANNVSSKLYLDEHVSSMVRLKHDEYLSLLRELARAEKKADDELATQLREKLVAAEREMTDQELAICKYATEMSTQSLSYRFVTELETKIFAKILDCLKQGVYATFNSDSRDVKRGERAIRNYKKGMPIPFAWDKSVRIEYEEKEKDFFLRWYNDIRFKFHFGRDRSNNRLIVSRCMKLDKDYEGDYQLCNSSIQIVKRDGSTKYFLLLVVKIPQEHVELNKKIVVGVDLGINYPAFAATNCTEERMSIGDREHFLNTRMQFQRRFKSLQRLKGTTGGKGRNKKLEPLERLRKAEHNWVHTQNHLFSLKVVNFAVQAHAATIHLEDLSGFGKDDDGNADERKEFVLRNWSYYELQNMIKYKAKKFGIQVEKIRPAYTSQTCSWCGQRGFRQGITFICENPECKKCGEKENADYNAARNIANSKDIIKDKDE

SEQ ID NO: 45 - protein, 396 aa, unidentified
  REGION 1..396: note = uncultivated organism
  REGION 1..396: note = MG119-16 Effector
MPKITRKIEIVPDIEGLTHEESNKKCYKTFYNFDRKLYKVANLLVSQLYGLDNLLSLMRLQNDEYVKCQSKLSLKSTTDTTKEDIKKRMKEIDAKLVSIKNDIAPKHPQTYSYRAITSSEYAKDVPSDILNTLKQDVYQHFNENKKEQIRGERSLTTYKKGMPIPFSLEKKHVIVCDGDNYYLPWFEDARFRLNFGRDRSNNRAIIDNCIKTKKYKMCAAAKIQLKDRKLFLLVTVDIPETENVPVKGKVMGVDLGVVNPAYVAVNDGPERSRIGNGDAFQKQRDVFRRRFRELQRLQLTQGGHGRKHKTKATEALRGKERNWVQTENHRISREIVNLAVRWKVETIQMESLKGFGKNQEGEVENNHRRLLGRGLISNFRKTSSIRLPWQVLQYNM

SEQ ID NO: 46 - protein, 489 aa, unidentified
  REGION 1..489: note = uncultivated organism
  REGION 1..489: note = MG119-17 Effector
MPIITRKIELKIVKDGLTDEEYDQQWKYLYQINNTIYLAANRISTHCLFNDEYEMRLRLPYKSRYDKINKGLENIKTELEKLNTKEKTSDKEKRDRLLNKQRQLVDERNELDEDVKNKKKDFFQCSKQNSTYQLASKEFLKYIPAEILTDLNRYVQNNHSNNKNKVKSGERALSTYKKGMGIPFSIKSESGLRLFVKEEGIYLKWFKGILFKLEFGKDSSNNRCIVERLIESYKQQKKKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 47 - protein, 475 aa, unidentified
  REGION 1..475: note = uncultivated organism
  REGION 1..475: note = MG119-18 Effector
MPTITRKIELKLCTEGLSEEERKAQWKLLYHINDNLYRSANNISSKLYLDSHVGSMVRLKHKDYLETLTALSKAQKKDDTALITELEARLSLFKSEMSDQEIAICKYATEMSERTLADHFASELELDIYSQILTQVKDVVQKYFKDDSKEVSQGLRSIRTYKKGMPIPFPWRNNIWLEAVEAKKQRGNDKKYDYFIKWYGGIRFQLMFGKDRSNNEQIVRRCLKMDENCNDNYQMRTSSIQMKKGAHGTELFLLLVVDIPQEQHKLNNKIVVGVDLGINVPAYVATNVTYDRCAIGSSEGFLAKRNEFRNRFRSLQKLKGTAGGRGRTKKLEPLERLRDKERNWVHTQNHLYSRDVVNFAIKVKAATIQMEDLKGYGKDENGEVVEEKKFLLGRWSYYELQKLIQYKAEKAGMKVNYINPRYTSQTCSCCGVRDPEARNGVHFICRNPACEMYGKEEHADYNAAVNIARSKDINK

SEQ ID NO: 48 - protein, 453 aa, unidentified
  REGION 1..453: note = uncultivated organism
  REGION 1..453: note = MG119-19 Effector
MSKITRKIELIPDIENLTHEESNQRCYKVFYNIDNKLYKVANLLVCQLFGLDNLLSLMRLQNDEYVKCQSKLASKSISEETKRDIKKRMEAIDKELLARKDEIAPKHPQTFAYRAIKDSDYAKDLPSDIFNTLKQDVFKHFNETKKEQLRGERSLTTYKRGIPVPFNLMKKNVIVSDGDNYYLTWFEETRFRLNFGKDRSNNRDIIDNCLKTNKYKLCTAAKIQLKNKKLFLLVTVDIPETKNKIIKGKVMGVDLGVVHPAYVAVNDGPERSLIGDGDAFQKQRDVFRRRFRELQRCQLTQGGHGRKHKTKATENLKGKERNWVQTENHRISREIINLAIRWRVETIQMENLKGFGKDSDGEVGTKHQRLLGRWSYFELQKDIEYKAAMAGIKVVYVNPAYTSQTCHVCGERGDRTERDTFICTNTGCDCYGKPQDADMNAAINIARSKDMIK

SEQ ID NO: 49 - protein, 462 aa, unidentified
  REGION 1..462: note = uncultivated organism
  REGION 1..462: note = MG119-20 Effector
MPIITRKIELKPDIDGLSNEESKNKCYELLRELDNTIYKAANLLVGQMFGMEEFVTMMRLQNDEYVTICKQMKLKKTTEDQRKQLKVRLDEIDAQINVQRQEMAPKGMRTFAGRAVKRSEYANSVPGEILECLKEVVFRHYSDVTKERRKGERTLSTYKKGMPIPFRFWGAQRLVQSEDKFYLQWYNGTRFLLDFGKDRSNNKLIVQRCLGIAEDEISYKTMSSLQLKKNKIFLLLNVEIPKTKFSPIEGKVMGVDLGIVWPAYVAVNNGPEHEHIGDGKNIQNMRNMFWRRFRELQRCQLTQEGHGRKHRTKALEHLHEKERNWIKTENHRISHRIVELAKRWQVETIQMENLKGFGKDKNGNVDDQHKRVLERWSYFELQKDVEYKADIAGIKVRYVNPAYTSLRCSKCGKVGVRPERELFFCTNVECDCYNRSIKADENAARNIAKSNDIIENKDNVEN

SEQ ID NO: 50 - protein, 344 aa, unidentified
  REGION 1..344: note = uncultivated organism
  REGION 1..344: note = MG119-21 Effector
MPTITRKIELKLYTEGLSEDERKAQWGLLYHINDNLYKAANNISSKLYLDDHVASMVRLKHDEYLSLVKALKKAKRQKSPDEDLIAELDKQVSVAEKEMTDQELAICKYATEMSTQSLSYRFATEIEVNIFAKILDCLKQGVYATFNSDAKDVKRGERAIRNYKKGMPIPFAWDDSLKIETEDKEFFLRWYNGLRFRLEFGKDRSNNRLIVKRCLKMDSDFDGEYKLCNSSIQFVKRDGGTKLFLLLVVNIPQENVELNKKIVVGVDLGINSPAYVATNITMERQQIGRRDTFLNARMAFQRRYKSLQKLQNTAGGRGRKKKLEPLERLKDAERNWVRTQKPPF

SEQ ID NO: 51 - protein, 478 aa, unidentified
  REGION 1..478: note = uncultivated organism
  REGION 1..478: note = MG119-22 Effector
MPTITRKIELDLCKDGLSEEQQKEQRLLLYHINDNLYKAANNVSSKLYLDEHVSSIVRLKHAEYLDLLKELAKAEKKNNADLIAELKKKLAAAEKEMTDQELAICKYATEMSTDTLSYRFANEIETKVFGQILATMKQGVHANFQNESKDVKRGKRSIRTYKKGMAIPFPWNDSIRLEYQKKESKKGLGEDGEYEFYLYWFKGIKFQLRFGKDRSNNRLILKRCFKLDENCDDDYHIKTSSIKMVKGSKGTEYYLFLVMDIPQEKHVLNKKIVVGVDLGINFPAYVATNITEERKAIGDRDHFLKARMAIIRRKQSFQRLTGTTGGRGRTKKLEPLERLKEKERNWVHTQNHLFSRDVIKFALQVKAATIQMEDLSGYGRDDEGNVEESKKFLLGKWSYYELQSMIKYKAQKAGIKVNFVNPAYTSQTCSECGKIQPQARNSVHYICENPECKNHGKDIQADYNAARNIARSKNIVKS

SEQ ID NO: 52 - protein, 467 aa, unidentified
  REGION 1..467: note = uncultivated organism
  REGION 1..467: note = MG119-23 Effector
MLTITRKIELHLCTEGLTDEQQKEQRLLLYRINDNLYKAANNVSSKLYLDEHVSSMVRLKHDEYLSLLRELARAEKKADDELTTELREKLAAAEREMTNQELAICKYATEMSTDTLAYRFANEIETQVFGQILACLKQNVHSTFNKDAVDVKRGERAIRNFKKGMPIPFPWSKSIKIEEEKNDFYLRWYNDIRFKFHFGRDRSNNRLIVSRCLKLDKDYEGEYQLCNSSIQIVKRDGSTKYFLLLVVKIPQEHVELNKKIVVGVDLGINYPAYVATNCTEERMSIGDREHFLNTRMQFQRRYKSLQRLKGTAGGKGRSKKLEPLERLRNAERNWVHTQNHLFSLKVVNFAVQTHAATIHLEDLSGFGKDDDGNADERNEFVLRNWSYYELQSMIEYKAKKYGIKVEKIRPAYTSQTCSWCGQRGFRQGVTFICENPECKKCGEKENADYNAARNIANSNDVIKEKNE

SEQ ID NO: 53 - protein, 484 aa, unidentified
  REGION 1..484: note = uncultivated organism
  REGION 1..484: note = MG119-24 Effector
MPTITRKIELRLCSEGLSEEERKAQWSLLYHINDNLYRSANNISSKLYLDEHVSSLVRLKHKEYKKLIKDLAKAKKQKIADAKAIAEMEDKLKSYEHEMTEQELAICKYADEMSSLSLAYGFATELELKIYAQILTQIQSKVHSDFQNDQKEVREGKRSIRTYKKGLPIPFPWNDTIKIEVVKKNNTEEQAKKSRRDNEEYDFYLNWYNGMRFRLHFGKDRSNNYQIVKRCFKLDEYCLDNYQLKTSSIQLVRKNKKTDFYLLLVVDIPQEKYSLNNQIVVGVDLGINVPAYVATNVTEDRKSIGNREHFLNARMAIKRKFHSLQRLEGTAEGRGRKKKLEPLERLREKERNWVHTQNHLFSRDVIKFALQAKAATIQMEDLSGYGKDDEGNVEEGKKFLLGNWSYYELQSMIKYKAQMVGMKVNFVAPAYTSQTCCCCGVKDEKNRNSTSFVCHNPQCTMYMKDIHADYNAARNIAKSKEIVK

SEQ ID NO: 54 - protein, 326 aa, unidentified
  REGION 1..326: note = uncultivated organism
  REGION 1..326: note = MG119-25 Effector
MERGIRTIPNFKKGIPVPFSIKPNGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRSLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQSTFICKNPDCTKGKGKQKSDGAYEGINADWNAARNIARSNEFVGKKKK

SEQ ID NO: 55 - protein, 469 aa, unidentified
  REGION 1..469: note = uncultivated organism
  REGION 1..469: note = MG119-26 Effector
MPIITRKIELKIVRLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 56 - protein, 481 aa, unidentified
  REGION 1..481: note = uncultivated organism
  REGION 1..481: note = MG119-27 Effector
MATEYTCITRKIEVHLHKHGDDEESVSRYKIEYQKWDEINDNLFKAANRVVSHHFFNDAYEYRLKLQSKRFRDIEKQLRTSRKNNLVDEEITALRAERDKLSAEFKKQRLEFLGVSEQHSTYRVITNEFIDVIPSDVLTCLNQNISLTYKNYAFDVERGIRTIPNFKKGIPVPFSIKGHGQLLLKKREDGSVYIRFPLGLEWDLRFGRDRSNNREIVERILSGEYDAGNSSIQETRNGKRFLLLLVKIPKEKHVLNPNRVVGVDLGINIPLYAALNDNEYGGMSIGSREQFLKMRMRMFAQKRELQSNLRHSTNGGHGRGQKLQALERVEGKERKWVRLQNHIFSKSIIDYALKNGAGVIQMERLTGFGRDKNDEVDEESKFILRYWSFFELQTMIEYKAKAAGIEIRYVNPYHTSQTCSFCGHYEKGQRINQATFICKNPDCIKGRGERKADGTFEGINADWNAARNISLSREFMNRKKK

SEQ ID NO: 57 - protein, 488 aa, unidentified
  REGION 1..488: note = uncultivated organism
  REGION 1..488: note = MG119-28 Effector
MATEYTCITRKIEVHLHRHGEYEEAKQRLIDDYRVWDTINDNLYKAANRIVSHCFFNDAYEYRLKIHSPRFQEIEKLLKYPKRNKLTDEDIKQLKAERKQLFADFKKQRHTFLRGGVAEGANPEQNSTYKVISNEFLEVIPSEILTNLNQNISSTYKNYSLDVERGIRTIPNYKRGIPVPFSIKQRGELMLKSRDDGSIYVRFPLGLEWDLSFGRDRSNNREIVERVLSGQYDVGNSSIQESKNRKRFLLLVVKIPKENHNLNPDRIVGVDLGINIPLYAALNDNDYGGMGIGSREQFLNMRMRMDAKKRELQRNLRQSTNGGHGRKQKLQALERFEGKERNWVHLQNHIFSKSIIEYAVKNNAGAIQMERLTGFGRDKNDEVDSNFKFILRYWSFFELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 58 - protein, 493 aa, unidentified
  REGION 1..493: note = uncultivated organism
  REGION 1..493: note = MG119-29 Effector
MIHLHFQNRTIIMTIITRKIELKHCKENLSEEERISQKKLLLHINDNLYRAANNISSKLYLDEHVSSLVRLKHKEYKDLVRDLAKAKKQKSHDEASIAEMEARLKGYEDEMTSQERAICKYTDEMSPLSLAYGFATELELDIYAQILTQIQSKVHKDFTNDNEDVRDGKRSIRTYKKGMPIPFPWNNAIRIEAIEKAKTEEKEVKNRRNREDYDFYLNWYNGIRFRLHFGKDKSNNYQIVKRCLKLDENCRDNYQLKTSSIKMENGTKKMYLLLVVDIPQEKYKLNNNIVLGVDLGINVPAYVATNITEDCKAIGNREHFLNARMAISRRFHSLQRLQGTAEGRGRKKKLEPLEHMREKERNWVRTQNHLYSRDIVNFAIKVKAATIHMEDLSGYGKDDEGNVEEDKKFLLGKWSYFELQTMIKNKAQKAGIRVNMVKPAYTSQTCSWCGERGIRDGLSFFCQNPECKQYAKKDIHADYNAARNIARSKDIVK

SEQ ID NO: 59 - protein, 308 aa, unidentified
  REGION 1..308: note = uncultivated organism
  REGION 1..308: note = MG119-30 Effector
MEIPFSIRENKRLKLFIKEKGIYLKWFKEILFRLEFGKDASNNRCIVERLIESDKQQKNKGEDYVANNSSIKLVKNEKNTRIFLLLSIDIPAKKQVLDKEVVLGVDLGIKCPLYLAINKNDNFKMQIGDIEHFHNQRTMFHKRFKSLQKLMCTQGGHGRKKKLEPLEKLKEKERNWVHTQNHVYSREVIKQALKQNAGIIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 60 - protein, 453 aa, unidentified
  REGION 1..453: note = uncultivated organism
  REGION 1..453: note = MG119-31 Effector
MSIVTRKIELIPDIENLTHEESNQRCYKLLYNIDKKLYKLANLLVCQLFGLDNLLSLMRLQNDEYVKFQSKLASKSISKETQKNIKERMKEIDKELLARKAEIAPKSPLTFAYRAIKGSLYAKDLPSDIFNTLKQDVFKHFNETKKEQLRGERSLATYKRGIPIPFSLMKKNVIVSEGDNYYLTWFEETRFKLNFGKDRSNNRAVIDNCLKTNKYKLCTAAKIQLKNKKLFFLVTVDIPETKNTIIKGKVMGVDLGVVHPAYVAVNDGPERSLIGDGDAFQKQRDVFRRRFKELQRCQLTQGGHGRKHKTKATEILRGKERNWVQTENHRISRKIVNLAIRWKVESIQMENLKGFGKDSEGEVETKHKRLLGRWSYFELQKDIEYKAQMAGIKVVYVNPAYTSQTCHVCGKKGDRTERDTFICLNTECSCYGKPQDADMNAAINIARSKNIVK

SEQ ID NO: 61 - protein, 488 aa, unidentified
  REGION 1..488: note = uncultivated organism
  REGION 1..488: note = MG119-32 Effector
MATEYTCITRKIEVHLHRHGEGEEAIERMKREYQIWDDINNNLYKAANRIVSHCFFNDAYEYRLKIHSPRFKAIEKALAYAKRDKLTPDEVKQLKAERSALFKQFKSQRQTFLRGGEEEGSNPEQNSTYQVITKEFKDYIPSEVLTNLNQNIAGVYKQFSLEIQNGNRSIQNFKKGIPVPFAIKSHGVLMIKKREDGTIYVRFPLGLEWDLAFGRDRSNNREIVERILSGAYDVGNSSIQEAKNGKRFLLLVVKIPKEINKLDKNRVVGIDLGINIPISAALNDNAYGGMSVGSREQFLKVRMRMAAQKRELQRSLRSTTNGGHGRKQKLQALDRLEGKERNWVHLQNHIFSKTIIDYAIKNGAGVIQMENLSNFGHNKFDEVNEGYKFILRYWSFFELQTMIEYKANAAGIDVRYVNPYHTSQTCSFCGHYEKGQRINQSTFVCKNPECTKGKGKQRGDGTFEGINADWNAARNIALSNDIVAKKKK

SEQ ID NO: 62 - protein, 469 aa, unidentified
  REGION 1..469: note = uncultivated organism
  REGION 1..469: note = MG119-33 Effector
MPTITRKIELKLCTEGLSDQERKDQWNLLYHINDNLYRAANNISSKLYLDDHVGSMVRLKHAEYLSLLRALEKAKKQKAPDEELIAELSQQVATAEQEMDEQAKAICQYATEMSTQTLSYRFATEMETNIFGQILTCLRQGVFSTFNSDARDVKRGERSIRTYKKGMPIPFPWNDSLRIELENGEFFLRWYNGMRFRFDFGKDRSNNRLIVQRCMKMDEDYEGEYKLCNSSIQMVKREGKPKFFLLMVVNIPQEQVELNKKIVVGVDLGINAPAYVATNITPERKQIGDREHFLNERMAFQRRFKSLQRLKGTTGGRGRAKKLEPLERLRKAEQNWVHTQNHLFSREVIDFAVKARAATIHIEDLSGFGKDQDGNADEKKEFVLRNWSYYELQNMITYKAAKYGIKVEKIRPAYTSKTCSWCGHQGFREGITFICENPECKKFGEKEHADYNAARNIANSKEIIKNNEE

SEQ ID NO: 63 - protein, 471 aa, unidentified
  REGION 1..471: note = uncultivated organism
  REGION 1..471: note = MG119-34 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 64 - protein, 483 aa, unidentified
  REGION 1..483: note = uncultivated organism
  REGION 1..483: note = MG119-35 Effector
MPLITRKIEVQIYSKGVSAEDYNSQWAFLRQINNYLYLAANLLASHNYFEEILSDRLKVTDTEYYKKRIKMSRQSGSGKEESKKELREIDKEVRNKINELFKGTTKRTELYRIVAEKFGEVIPTKILANLNKSVTDCYVGEEKDIRKGERSLRNYKKGLPIPFSWGKKEPTFPYIVKNEETGNSDILFQFFPDLSKKFCKRQNPGSAGSPLTFRMHFGKDKSNNRAIVERIIADKDKAGDDRYKLSQSSLQIVGKDKNTKFFLLLCVDIPAQKNHLDPDKVLGIDLGINYPVYYATNKEDNIKGHIGDRDSFLKQRLAFQRRYKALQSVTCTSGGRGRKKKLQPLESLRSKERAWTKHMNHLYSREIIKAAQKLGTGTIHMEELTGIGKDEDGNVKKKRQFILRNWSYFELQTMIQYKAKMAGITVKKIPPAYTSQTCSCCGEKGERKEQAHFKCLNPLCSMFDKDVNADYNAARNIAKYQAD

SEQ ID NO: 65 - protein, 468 aa, unidentified
  REGION 1..468: note = uncultivated organism
  REGION 1..468: note = MG119-36 Effector
MPTITRKIELTLCTDGLSDEERKAQWGLLYHINDNLYKAANNISSKLYLDEHVSSMVRLKHDEYLSLQKELAKAERQKMPDVDVIEELRERLSAVEQEMSDQELAICKYATEMSTNTLAYRFATEIETNIFGQIMARLENNAQAVFLTDAPDVKRGERAIRNYKKGMPIPFPWNNSIKIECEGGEFYLRWYNGLRFHFNFGKDRSGNRLIVQRCLKLDKEYDGEYKLCNSSIQMVKRDGSTKFFLLMVVNIPQEYVELNKHIVVGVDLGINVPAYVATNITPERKAIGDREHFLNTRMAFQRRYKSLQRLKTTAGGKGRTKKLEPLERLRQAEHNWVHTQNHLFSREVVNFALQTHAATIHLEDLSGFGKDSDGNADERKEFVLRNWSYYELQNMITYKAAKYGIRVEKIRPAFTSRTCSCCGHEGFREGVTFICENPECQQFGEKVHADYNAARNIANSKDVIKKNE

SEQ ID NO: 66 - protein, 469 aa, unidentified
  REGION 1..469: note = uncultivated organism
  REGION 1..469: note = MG119-37 Effector
MPTITRKIELVLCTEGLSDEERKAQRGLLYHINDNLYKAAKNISSKLYLDDHVGSMVRLKHDAYLSLLRNWEKAKKQKTPDEELIEELGKQVAEAEKEMTDQELAICKYATEMSTQSLSYRFATELETNIFAKILDCLKQGVFATFNSDARDVKRGERAIRNYKKGMPIPFAWSDSLRIEAEKNDFYLRWYNGLRFYLNFGKDRSNNRMIVRRCLNLDEDFEGEYKLCNSSIQMVKRDGAPKIFLLLVVNIPQEHVELNKKIVVGVDLGVNVPAYVATNITDERKAIGDREHFLNTRMAFQRRYKSLQRLRGTAGGKGRAKKLEPLERLRKAEHNWVHTQNHLFSREVVNFAVQARAATIHMEDLSGFGKDSDGNADEKKEFVLRNWSYYELQTMIAYKAAKYGIKVEKIRPAYTSKTCSWCGQQGFRQGVTFICENPDCKQCGEKVHADYNAARNIANSKDVIKDKNE

SEQ ID NO: 67 - protein, 497 aa, unidentified
  REGION 1..497: note = uncultivated organism
  REGION 1..497: note = MG119-38 Effector
MPTITRKIELKIVKDRLTDEKERLTDEEYDQQWKYLYQINNTIYQAANRISTHCLFNDEYEMRLRLSYKSRYDKINKGLENIKTELEKLNTKKKTSDKEKRDRLINEQRQLVDERNELDEDVKNKKKDFFQCSKQNSTYQLASKEFLKYIPAEILTDLNRYVQNNHNNNKNKVKSGERALSTYKKGMGIPFSIKPESGLRLFVKEEGIYLKWFKGILFKLEFGKDTSNNRCIVERLIESDKQQKKNKGEDYVANNSSIKLIKKGNDKSTRIFLLLSIDIPAKKQVLDKDVVLGVDLGIKCPLYLAINKDDNFKMQIGDIEHFHNQRTMFQKRFKSLQKLMCTQGGHGRKKKLEPLEKLKEKERDWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 68 - protein, 135 aa, unidentified
  REGION 1..135: note = uncultivated organism
  REGION 1..135: note = MG119-39 Effector
MQSAIGYERKNHLFSRDVVRFAVKARAASIHMEDLSGFGKDNDGNADDKKEFVLRNWSYYELQTMIKYKAAKYGIKVEKIRPAFTSQTCSWCGQPGFRRGEIFICENPACEKCGEKENADYNAARNIANSKDIIK

SEQ ID NO: 69 - protein, 479 aa, unidentified
  REGION 1..479: note = uncultivated organism
  REGION 1..479: note = MG119-40 Effector
MPIITRKIELKIVKDGLTDEEYDQQWKYLYQINNTIYLAANRISTHCLFNDEYEMRLRLRYMSQCNKIDKKLEKLNPDKKTSDKEEQYRLLNERKELDEDFKNKKQDFQRIKRNSTYQLVSKEFLKYIPAEILTDLNQYVQNNYNNNKREVKSGERALSTYKKGMGIPFSIKPESGLRLFIKENGIYLKWFKLNNDRKEKEILFRLEFGKDASNNRCIVERLIESDRQQKKKGEDYIANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRCGEKINADFNAARNIAMSKKIVER

SEQ ID NO: 70 - protein, 468 aa, unidentified
  REGION 1..468: note = uncultivated organism
  REGION 1..468: note = MG119-41 Effector
MPSITRKIELTLCTEGLSDEKRKEQWGLLYHINDNLYKAANNISSKLYLDDHVSSMVRLKHADYLSLLRELARAEKQKTPNIDLIAELREKVSTAEKEMTDQELAICNYATEMSTKSLSYRFATEIETDIFAKILDCLKQGVYAKFTSDTRDVKNGERAIRNYKKGMPIPFAWNDSLRIEEENGEFYLRWYNGLRFLLYFGKDRSNNRMIVKRCLKMDEEFDGEYKLCNSSIQIVKREGRTKLFLLLVVNIPQENVELNKNIVVGVDLGLNYPAYVATNCTEEHKYIGDRDHFLKIRMQFQSRFRSLQRLRGTAGGKGRAKKLEPLERLRQAECNWVHTQNHLFSRDVVNFAVQTRAATIHMEDLSGFGKDPDGNAEEKKEFVLRNWSYYELQNMIKYKAKIYGIRVEKVRPAYTSKNCSWCGHLGFRESTTFICENPECKQCGKKIHADYNAARNIANSKDIIKENE

SEQ ID NO: 71 - protein, 501 aa, unidentified
  REGION 1..501: note = uncultivated organism
  REGION 1..501: note = MG119-42 Effector
MPIITRKIELKIVKDGLTDEKYKLTDEEYDQQWKYLYQINNTIYQAANRISTHCLFNDEYEMRLRLHMPRYKEIDEGLENIKTELEKLNTKKKASDKEKRDRLLNEQRQLVDERKELDKDVKNKKKDFLQCSKQNSTYQLVSKEFLKYIPAEILTDLNQYVQNNYNNNKRKVKSGERALSTYKKGMGIPFSIKPESGLRLFIKENGIYLKWFKLNNDRKEKEILFRLEFGKDASNNRCIVERLIESDRQQKKKGEDYIANNSSIKLVKNGKNTRIFLLLSIDIPAKKQVLDKDVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHIESLKDFGKGKDGYVKDEYKYLLRYWSYYELQSMIEYKAKLEGIEVKYIDPAYTSQTCSYCGERGERKKQEEFVCTNPQCKRCGEKINADFNAARNIAMSKKIVKDN

SEQ ID NO: 72 - protein, 472 aa, unidentified
  REGION 1..472: note = uncultivated organism
  REGION 1..472: note = MG119-43 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKKVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 73 - protein, 470 aa, unidentified
  REGION 1..470: note = uncultivated organism
  REGION 1..470: note = MG119-44 Effector
MPIITRKIELKIVRLTDEEYDQQWKYLYQINNTIYQAANRISTHCLFNDEYEMRLRLRYMSQCNKIDKKLEKLNPDKKTSDKEEQYRLLNERKELDEDFKNKKQDFQRIKRNSTYQLVSKEFLNYIPAEILTDLNQYVQNNHNNNKNKVKSGERALSTYKKGMGIPFSIKPESGLRLFVKEEGIYLKWFKGILFRLEFGKDASNNRCIVERLIESDKQQKKKGEDYVANNSSIKLVKNGKNTRIFLLLSIDIPAKKQVLDKEVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 74 - protein, 486 aa, unidentified
  REGION 1..486: note = uncultivated organism
  REGION 1..486: note = MG119-45 Effector
MPIITRKIELKIVRLTDEEYDQQWKYLYQINNTIYQAANRISTHCLFNDEYEMRLRLSYKSRYDKINKGLENIKTELEKLNTKKKTSDKEKRDRLINEQRQLVDERNELDEDVKNKKKDFFQCSKQNSTYQLASKEFLKYIPAEILTDLNRYVQNNHNNNKNKVKSGERALSTYKKGMGIPFSIKSESGLRLFVKEEGIYLKWFKGILFKLEFGKDSSNNRCIVERLIESYKQQRKKGEDYVANNSSIKLIKKGNDKSTRIFLLLSIDIPAKKQVLDKDVVLGVDLGIKCPLYLVINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRCGEKINADFNAARNIAMSKKIVER

SEQ ID NO: 75 - protein, 453 aa, unidentified
  REGION 1..453: note = uncultivated organism
  REGION 1..453: note = MG119-46 Effector
MSKITRKIEIIPDIDGITHEESNKKCYNTFYKFDRKLYKVANLLVSQLYGLDNLLSLMRLQNDEYVKCQSKLSFKSITDATKEEIKKRMQEIDAELVSMKNDIAPKHPQTYSYRAVISSEYAKDIPSDILNNLKQDVYQHFNENKKELIRGERSLATYKKGMPIPFSLEKGHVIICDGDNYYLPWFEDIRFRLNFGRDRSNNRAIIDNCIKTKKYKLCASAKIQLKERKLFLLITVDIPKANSVPVKGKVMGVDLGVVNPAYVAVNDGPERSRIGNGETFQKQRDVFRRRFRELQRSQLTQGGHGRKHKTKATEILRGKERNWVQTENHRISREIVNLASRWKVETIQMESLKGFGKNQEGEVENNHKRLLGRWSYFELQKDIEYKAAMAGIKVQYVNPAYTSQTCHVCGQRGNRIEREIQGSNGWYQGTICKSCIYQPNLSCLWSTRKQNRT

SEQ ID NO: 76 - protein, 469 aa, unidentified
  REGION 1..469: note = uncultivated organism
  REGION 1..469: note = MG119-47 Effector
MPTITRKIELHLCTDGLSEEQRKEQWGVLYHINDNLYKAANNISSKLYLDEHVSSMVRMKHAEYLSLVKELARAEKQKTVDEETIEELRDKLATAEKEMTDQEKAICKYATEMPTNTLAYKFANEIETDIFGQILACLDNNAHAVFVDDAPTVRKGERAIRNYKRGMPIPFPWNKAIRIDVSEGELYLKWYHGIRFHLFFGKDKSNNRLIVNRCLKLDKDYEGDYKLCNSSIQIVKREGAVKLFLLLVVNIPQEHVELNKKIVVGVDLGVNVPAYVATNITEERKAIGDREHFLNTRMQFQRRYKSLQRLKATTGGKGRAKKLEPLERLRKAEHNWVHTQNHLFSREVVNFAVQTHAATIHLEDLSGFGKDNDGNADEKKEFVLRNWSYYELQNMIAYKAAKYGIKVEKVKPAYTSKTCSCCGQIGFRQGVTFICENPACKQCGEKVHADYNAARNIANSKDVKKDKNE

SEQ ID NO: 77 - protein, 495 aa, unidentified
  REGION 1..495: note = uncultivated organism
  REGION 1..495: note = MG119-48 Effector
MPTITRKIELKIVKDRLTDEKERLTDEKYDQQWKYLYQINNTIYQAANRISTHCLFNDEYEMRLKLHMPRYKDIEKKLEEIEKKQKGLNTKKKASDKEERDRLLNEKQQLVNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 78 - protein, 410 aa, unidentified
  REGION 1..410: note = uncultivated organism
  REGION 1..410: note = MG119-49 Effector
MVRMKHAEYLSLLSELERARKKTDEELVEVLSNRVAAAEKEMTDQELAICQYATEMSTDTLAYRFANEIEIKVFGQILACLKQGIHSNFKKDAVDVKRGERAIRNYKKGMPIPFPWSKSIRIESEGSEFYLRWYNGIRFRFTFGKDRSNNRLIVRRCLKMDDDFEGEYKLCNSSIQLAKRDGAPKLFLLLVINIPQEHVELNKKIVVGVDLGINAPAYVATNITEERKQIGDRDHFLKTRLTFQRRYKSLQRLKGTSGGKGRTKKLEPLERLRKAERNWVHTQNHLFSRDVVDFAVQTRAATIHMEDLSGFGKDDDGNADEKKEFVLRNWSYYELQNMITYKAAKNGIRVEKIRPAYTSQTCSCCGEKDPRNRDGISFICHNPNCEQYDKEVHADYNAARNIAKSNDIVE

SEQ ID NO: 79 - protein, 471 aa, unidentified
  REGION 1..471: note = uncultivated organism
  REGION 1..471: note = MG119-50 Effector
MPTITRKIKLELCTKGLSEEERKAQWNLLYHINDNLYRSANNISSKLYLDEHVSSLVWLKHKEHQTLKADLAKAKKQKIQDEKTIAELESRLKSCESEMSDQELAICKYTDEMSSKTLSYKFATELELNIYAQILNQVQSKVYADFQNDQKDVRDGKRAIRTYKKGMPIPFPWRNNIRMEPVKKGREYEFYIKWYNDIRFQLIFGKDRSNNRLILQRCFKLDENCVEDYQMRTSSIKMVKGANGTELFLYLVVDIPQEKHTLNNKIVVGVDLGINVPAYVATNVTDDRKAIGDREHFLNTRMAISKRFHSFQRLKGTTGGRGKTKKLEPLERLKEKERNWVHTQNHLFSRDVITFALHVKAATIQMEDLSGYGKDDEGNVVEEKKFLLGKWSYYELQDMIKYKAKKVGMRVNFIKPAYSSQTCSWCGERGERNSTSFVCTNSECSHYGEDLHADYNAARNIARSKNIIRYE

SEQ ID NO: 80 - protein, 488 aa, unidentified
  REGION 1..488: note = uncultivated organism
  REGION 1..488: note = MG119-51 Effector
MATEYTCITRKIEVHLHKHGDGEEAEQRRAEEFRIWNEINDNLYKAANRIVSHCFFNDAYEYRLKIQSPRYNEIQKSLRYTKRNKLTDDEIKSLKAERKSLFDEFKKQRQTFLRGGVSEGANPEQNSTYKVVSNEFLDVIPSHILTCLNQNVSSTYKCYSKEVEFGNRTIPNFKKGIPVPFPIKHGNILLLKKREDGSIFVDFPKGLEWDLSFGRDRSNNREIVERILSGQYDAGTSSLQEGKNGKIFLLLVVKIPKQSNALDPNRVVGIDLGINIPLYAALNDNEYGGMSIGSREQFLKMRMRMTAQKRELQRNLHYSTNGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFICKNPECEKGKGKKRSDGTYIGINADWNAARNIALSDKFVDKKKK

SEQ ID NO: 81 - protein, 496 aa, unidentified
  REGION 1..496: note = uncultivated organism
  REGION 1..496: note = MG119-52 Effector
MPTITRKIELKIVKDRLTDEKERLTDEKYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFLKYIPAEILTDLNRYVQNNHNNNKNKVKSGERALSTYKKGMGIPFSIKPESGLRLFVKEEGIYLKWFKGILFKLEFGKDTSNNRCIVERLIESDKQQKKNKGEDYVANNSSIKLIKKGNDKSTRIFLLLSIDIPVKKQVLDKDVVLGVDLGIKCPLYLAINKDDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCERRGEKINADFNAARNIAMSKKIVKDN

SEQ ID NO: 82 - protein, 485 aa, unidentified
  REGION 1..485: note = uncultivated organism
  REGION 1..485: note = MG119-53 Effector
MPIITRKIELKIVKDGLTDEEYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 83 - protein, 497 aa, unidentified
  REGION 1..497: note = uncultivated organism
  REGION 1..497: note = MG119-54 Effector
MAEYTVITRKIEVHLHSDDNSEESKELLQEKYRAWNEINDHLYEAANRIVSHRFFNDEYEMRLRVHSPRYKEIKDILRKAQKDKLADSYVKELKAERKQLESEFREQRLTFLRGGDPDGTGSEQNSTYDVATNEFSDSIPSNILTCLNQNIIKTYNSYKTEIKFGERTIPNFKKGLPVPFPIKYNGNLQICKRDDGTIFVRLPGKKGQKWHLEWDLDFGRDRSNNREIVERILNGTYDVGNSSIQETRSRKRFLLLVVKIPKERNVSLDPNRVAGIDLGINIPLYAALNDNEYARSRIGSRDQFLDVRNRMEAQLHKLQANLRNSTAGGRGRGHKLQALERFKEKERNWVRLQNHIFSKGAIEFAVKNNAGVIQMERLTGFGRDGNGEVKNDRKFLLRNWSYFELQQLIEYKALAAGIEVRYVDPYHTSQICSFCGHYEPDQRSDQPHFICKNPECEKGKGKKLKDGSYEGINADWNAARNIAKSEKIVDRRKNTKK

SEQ ID NO: 84 - protein, 485 aa, unidentified
  REGION 1..485: note = uncultivated organism
  REGION 1..485: note = MG119-55 Effector
MPIITRKIELKIVKDGLTDEEYDQQWKYLYQINNTIYQAANRISTHCLFNDEYEMRLKLHMPRYKDIEKKLEEIEKKQKGLNTKKKASDKEERDRLLNEKQQLVNERKEIDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKGKGEDYVANNSSIKLVKNGKNTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 85 - protein, 467 aa, unidentified
  REGION 1..467: note = uncultivated organism
  REGION 1..467: note = MG119-56 Effector
MPTITRKIELTLCTEGLADEQRKAQWQLLYHINDNLYKAANNVSSKLYLDEHVSSMVRMKHSEYLSLVKELAKAEKQKKTDEDLIAGLRDRLTAVEREMTDQELAICKYATEMSTKTLAYKFATEIETNVFGQILACLKQAAQSNYNSDARDVKRGERAIRNYKKGMPIPFPWNDNLRIEPDGDEFFLRWYNGLRFHLIFGKDRSNNRMIVKRCLKMDKDFEGEYKLCNSSIQMAKSDGKPKLFLLLVVNIPQEHVELNKNIVVGVDLGVNVPAYVATNITEERKTIGDREHFLNTRMQFQRRYKSLQRLKATASGKGRTKKLEPLERLRKAEHNWVHTQNHLFSREVVNFAVQTRAATIHMEDLSGFGKDNDGNADEQKEFVLRNWSYYELQNMIAYKAAKYGIKVEKVRPAYTSKTCSWCGQQGFRQGVTFICENPACKQCGEKVHADYNAARNIANSKDVLNNS

SEQ ID NO: 86 - protein, 474 aa, unidentified
  REGION 1..474: note = uncultivated organism
  REGION 1..474: note = MG119-57 Effector
MAAITRKIELYIDKRNLTEDEYKAQWQYIRQIDNTLFLAANRISSHCLLNDELEIRLKLQMPEYCEIEKSLRNSKKNNLSKEEVSELKLRKKELDAIVKRQKDEFLRTSQNSTYQLVAREFANIPTDILTNLNNDIVSKYGKSRLDIINGVKSLSTYRKGLPIPFSVNKKFPFVFIDGEYYLEWFKGKTKEITGKPLQFKLHFGKDKSNNRTIVERIVESARLGKKKGEAYVVNNSSIQLVEKGNSSKIFLLLSLDIPTAKRTLDPNLVMGIDLGINYPIYYATNGNAYIKGHIGDRDSFLNERMVFQRRFKELQRLQCTQGGRGRQKKLAPLEKLRDKERNWVKTKNHIFSREIIKCALKIGAGIIHLEKLKNIGKDKDGNIEDSKKYILRNWSYHELQERIEYKAAMEGIMVKYVDPAYTSQTCSCCGNKGERISQSVFKCLCPECSEYGKEVNADYNAARNIAKSEIFVKK

SEQ ID NO: 87 - protein, 478 aa, unidentified
  REGION 1..478: note = uncultivated organism
  REGION 1..478: note = MG119-58 Effector
MPTITRKIELRLCTEGLSDEERKAQWMLLYHINDNLYRSANNISSKLYLDEHVSSMVRLKHAEYQSTAAELLKAKKNNADEGTISTLEDKVETLKTEMSAQGIAICNYATEMATRTLAGKFASELELNIYGQILAEVKNVVHTNFTNDAKEVREGKRSIRTYKKGMPIPFPWNKSIKIEPVKASSQNEGQDDYEFYLKWYNGLKFILHFGKDRSNNRQILKRCFGLDNLCNERYQMRTSSIQMKKGSNGMELYLLLVLSIPKEQHSLNKKVVVGVDLGINVPAYVATNCTEERRAIGDREQFLNTRMAIIRRKHSFQRLKGTAGGRGRKKKLDPLERLRETERNWVHTQNHLYSRDIIKFALETKAATIQMEKLKGFGRDDNGNVIEEKKFLLGKWSYYELQNMIKYKAGKVGIKVNFIAPAYTSQTCSCCGVRDDRNRKSTSFICHNPDCQMYGKEIHADYNAARNIARSKNVIKDE

SEQ ID NO: 88 - protein, 466 aa, unidentified
  REGION 1..466: note = uncultivated organism
  REGION 1..466: note = MG119-59 Effector
MPTITRKIELTLCTDGLSDDLRKDQWQLLYHINDNLYKAANNISSKLYLDEHVASMVRLKHAEYLGLIKELAKARKRADDEAVRDLCSKLAVAEQEMNEQAKAICDYATEMSTQTLSYNFAKEIETNIFGQILTCLRQGVLLNFNSDARDVKRGERAIRNYKKGMPIPFPWNDTIKIVSEGDEFYLRWFSGLRFHLNFGKDRSNNRMIVRRCLKMEQDFDEEYKISNSSIQVAKRDGKQKLFLLLVVQIPQEQVVLNKKIVVGVDLGVNVPAYVATNITEERKAIGDREHFLNTRMQFQRRYKSLQRLKTTEGGRGRAKKLEPLERLRKAEHNWVHTQNHLFSREVVNFALQTQAATINMEDLSGFGKDNDGNADECKEFVLRNWSYYELQNMIVYKASKYGIRVQKIRPAYTSKTCSWCGHMGFREGVTFICENPDCKQFGEKVHADYNAARNIANSKEIIKNDE

SEQ ID NO: 89 - protein, 483 aa, unidentified
  REGION 1..483: note = uncultivated organism
  REGION 1..483: note = MG119-61 Effector
MPTITRKIRLHLCTDGLSEEERKAQWKMLYRINDNLYRAANNISSKLYLDEHISSMVRLKHAEYTSLKTELLKAKKADDEETVAELEARINVLNAELSAQEEAICSYATEMATRTLAGKFASELDLNIYGQILAEVKSVVFKNFNSDSKDVREGKRSIRTYKKGMPIPFPWNKTIRLEAVKKESSSKHDEDEYEVYLNWYKSSRTEKKAIRFRLDFGKDKSNNQQIVKRCLNLDNTSSESYQLQTSSIQMKKGSEGAELYLLLVVNIPQDQHVLNKKIVVGVDLGINVPAYVATNCTEERKSIGDREHFLNARIAFHRRFHSFQKLKGTTGGRGRKKKLEPLERLREKERNWVHTQNHLISRDVINFALQTKAATIQMEDLSGYGKDEEGNVKPENKFLQSRWSYFELQSMIKYKAAKCGIKVNLINPSYTSQTCSWCGQMGVRESTSFVCQNPECKKYGKDIHADYNAARNIARSNKTVKNE

SEQ ID NO: 90 - protein, 490 aa, unidentified
  REGION 1..490: note = uncultivated organism
  REGION 1..490: note = MG119-62 Effector
MATEYTCITRKIEVHLHKHGDGEEAENRRAEEFRMWNDINDNLYKAANRIVSHCFFNDAYEYRLKIQSPRYREIQRKLRYSKRDKLSDAEIKELKAERNELDKEFRKQYQAFMLGGSSEGYKSTIPQNSTERIVNNEFGDIIPSNVLSCLNQNVFQTYKQYRTDVEFGKRTISNFKKGMPVPFSIKANKNLMLKKREDGSIFVYFPKGLEWDLSFGRDRSNNREIVERILSGQYDVGTSSLQEAKNGKVFLLLVVKIPKQSDKLDPNRVVGIDLGINIPLYAALNDNEYGGMSIGSREQFLKMRMRMVAQKRELQRNLRHSANGGRGRSHKLQALERLEGKERNWVHLQNHIFSKNIIEFAVKNNAGVIQMERLTGFGHDRNDEVDDGFKFILRYWSFFELQSMIEYKAEAAGIEVRYIDPYHTSQTCSFCGHYEKGQRIDQATFVCKNPECEKGKGKKRSDGTYTGINADWNAARNIALSDKFVDKKKK

SEQ ID NO: 91 - protein, 473 aa, unidentified
  REGION 1..473: note = uncultivated organism
  REGION 1..473: note = MG119-63 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFVKENGIYLKWFKGILFKLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 92 - protein, 467 aa, unidentified
  REGION 1..467: note = uncultivated organism
  REGION 1..467: note = MG119-64 Effector
MTIITRKIELKLCTDGLSDEERKMQWKLLYRINDNLYKAANNISSKLYLDEHVSSMVRLKHAEYLLLLKEIDKANRKKEPDVDVINGLNQKLNAIEVEMSDQAKAICKYATEMATKSLAYRFAQEFDLDIFGQILSCVQQNTHATFTKDMLDVSRGERSIRTYKKGMPIPFPWNKSIRITNKGEEFYLRWYKGIRFHLFFGKDRSNNRAIVKRCLKLDTDYQGEYKICNSSIQIVKKDDGVKLFLLLVVDIPKENRKLNNRIVVGVDLGINVPAYVATNITEERKAIGDREHFLNERMAFQRRYKSLQRLKNTAGGKGRKKKLEPLEALRKAEHNWVHTQNHLFSREVVNFAVQAEAATIHMEKLKGFGKDNEGNADERKEFVLRNWSYYELQNMITYKAAKYGIKVEYIRPAYTSRTCSWCGELGFREGVTFICENPNCKKCGEKIHADYNAARNIANSKEIEDNE

SEQ ID NO: 93 - protein, 468 aa, unidentified
  REGION 1..468: note = uncultivated organism
  REGION 1..468: note = MG119-65 Effector
MPTITRKIELTLLTEGLSEEQRKEQWGLLYHINDNLYKAANNISSKLYLDDHVSSMVRMKHAEYLSLLKELARAEKQKTPDADAIAELRKKVAAAEKEMTDQEHAICKYATEMSTQSLSYRFATELETNIFAKILDCLKQGVFATFNSDARDVKRGERAIRNYKKGMPIPFAWDKSLRIEKDNKDFYLRWYNGLRFLFNFGKDRSNNRLIVERCLKMDADYDGEYKLCNSSIQIAKREGKTKLFLLLVVKIPQEHVELNKKVVVGVDLGINVPAYVATNITEERKAIGDREHFLNSRMAFQRRYKSLQRLRGTAGGKGRAKKLEPLERLRKAEHNWVHTQNHLFSREVVDFAVKSHAATIHMEDLSGFGKDNDGNADERKEFVLRNWSYYELQNMIAYKAAKYGIKVEKIHPAYTSKTCSWCGQLGFREGVTFICENPECKQCGEKVHADYNAARNIANSKDIIKKNE

SEQ ID NO: 94 - protein, 383 aa, unidentified
  REGION 1..383: note = uncultivated organism
  REGION 1..383: note = MG119-66 Effector
MSTQEKAICKYTDEMSPLSLAYGFATELELDIYAQILTQIQSKVHKDFTGDSEDVRDGKRSIRSYKKGMPIPFPWNNTIRIEAVEKVKVEGKETKSRRDREDYDFYLNWYNGIKFRLNFGIDRSNNYQIVKRCLNLDENCRDNYQLKTSSIKMVNGTKEMYLLLVVDVPQEKYELNNNIVLGVDLGINVPAYVATNITEDRKAIGNREHFLNARMAISRRFRSLQRLQGTAEGRGRKKKLEPLEPMREKERNWVRTQNHLYSRDIVNFAIRVKAATIHMEDLSGYGKDSEGNVKEEKKFLLGKWSYFELQTMIKNKAQKAGIKINIVKPAYTSQTCSWCGERGLRKGTSFFCQNPECEQYANKDIHADYNAARNIARSKEIVK

SEQ ID NO: 95 - protein, 469 aa, unidentified
  REGION 1..469: note = uncultivated organism
  REGION 1..469: note = MG119-67 Effector
MPTITRKIELSLCTDGLSDEQLKEQRQLLYHINDNLYRAANNVSSKLYLDEHVSSMVRLKHADYLSLLRDLARAEKQKSPDEALISELRSKLAAAQREMTEQELAICRYATEMSTQSLSYRFVTEMETHIFAKILDCLKQGVYATFNSDARDVKRGERSIRNYKKGMPIPFAWSDSVRIEQEADEFYLRWYNGIRFRLVFGKDRSNNRLIVKRCLKLDKDYEGDYKLCNSSVQMVKREGKPKTFLLLVVKIPQEQVALNKKIVLGVDLGINYPVYAATNCTEERMYFGDREHFLNTRMQFQRRYKSLQRLKGTTGGKGRKKKLEPLERLRKAERNWVHTQNHLFSQKTVDFALQTHAATIHLEDLSGFGRDSDGSAEEKKEFVLRNWSYYELQQMITYKAAKYGIKVEKIRPAYTSQTCSWCGQRGFRQGVTFICENPECKKCGEKEQADYNAARNIAKSKDVIKDDDE

SEQ ID NO: 96 - protein, 468 aa, unidentified
  REGION 1..468: note = uncultivated organism
  REGION 1..468: note = MG119-68 Effector
MPTITRKIELTLCTQDLSDEQRKEQWGLLYHINDNLYKAANNISSKLYLDDHVSSMVRLKHADYLSLLKEQARAEKQKTPDVEKLAEVRAKLAAAEQEMSDQELAICKYATEMSTNTLAYNFAKEIETKIFGQILACLENNAHALFVDDSPNVRRGERAIRNYKKGMPIPFPWNKSIRIEAKDSEFYLRWYNGLRFLLTFGKDRSNNRMIVKRCLKLDKDYDGEYKLCNSSIQMVKREGKPKLFLLMVVNIPQEHVELNKNIVVGVDLGINVPAYAATNITEERKAIGDREHFLNTRMTFQRRYKSLQRLKTTAGGKGRTKKLEPLERLRQAEHNWVHTQNHLFSREVVNFAVQTHAATIHLEDLSGFGKDNDGNADERKEFVLRNWSFYELQAMIAYKAAKYGIKVEKIRPAYTSKTCSWCGQQGFRSGVTFICENPECKQCGEKVHADYNAARNIANSKEIVRNNE

SEQ ID NO: 97 - protein, 471 aa, unidentified
  REGION 1..471: note = uncultivated organism
  REGION 1..471: note = MG119-69 Effector
MPVITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDKQQKNKGEDYVANNSSIKLVKYGKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNARTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 98 - protein, 451 aa, unidentified
  REGION 1..451: note = uncultivated organism
  REGION 1..451: note = MG119-70 Effector
MSKITRKIEIIPDVEGLTHEESNEKCYKAFYNYDRKLYKVANLLVSQLYGLDNLLSLMRLQNEEYVDSQRKLSFKSTTDTAKEEIKKRMEEIDAELMAIKKKIAPMHPQSYSYRAVNSSEYAKDMPSDILDSLKQDVYKHFNDSKKEQIRGERSLTTYKRGMPIPFNLKKKHSIVCDGGNYYLLWFEDTRFRLNFGRDRSNNRAIIDNCIKKYKLCAAAKIQLKERKLFLLITVDIPKAESVPVKGKVMGVDLGVVNPAYVAVNDGPERSRIGNGEAFQKQRDVFRRRFRELQRSQLTQSGHGRKHKTKATEILRGKERNWVQTENHRISREIVNLASRWKVETIRMESLKGFGKNQEGEVENNHKRLLGRWSYFELQKDIEYKAAMAGIKVQYVNPAYTSQTCHVCGQRGNRIERDTFICTNPECTCYNQVQDADMNAAINIAKSNDVVK

SEQ ID NO: 99 - protein, 473 aa, unidentified
  REGION 1..473: note = uncultivated organism
  REGION 1..473: note = MG119-71 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRESKRLKLFVKENGIYLKWFKGILFKLEFGKDASNNRCIVERLIESDKQQKGKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 100 - protein, 463 aa, unidentified
  REGION 1..463: note = uncultivated organism
  REGION 1..463: note = MG119-72 Effector
MEQITLTRKIELFVNEEGNKKNECYDLLHYLNNNLYKIANDSVNYLHIKKNLPEFILKHNKEDYEKYVNNLNKMSEIKNFKSTEFIELKNQNRDIINKYSNNEEYKKMIYNDSKETTLVYHNINKRLPKGLSSISASLSKKIENDYKIKEKEYFLGKSTLNIYKRGLPIPFNISGKLVKDPSLFFKFDNGKYYINLFGYNFGLFFGPDRSNNKAIIEHIINDTDKYKLCDSAIQFKDKKIFLLLSFKQPLDNTIKLDDNKVLGVDLGVNAPVYLAINDNDYYKKQIGDIHHFDSHKLQYKKRYTSLIKNLEISNGGRGRKKKLSALDKLKKVEKNWTNTEQHKISNSVIKEALNNNCKYINIENLENYKENLTENKKYIMRIWGYYGIQEKIMYKAKKHNIVVRKINPAYTSQKCHKCGNIEKTARNKQIFTCLNEKCSIFGKEIHADFNGAKNISLSTEFKK

SEQ ID NO: 101 - protein, 471 aa, unidentified
  REGION 1..471: note = uncultivated organism
  REGION 1..471: note = MG119-73 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDENVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKEVVLGVDLGIKCPLYLAINKNDNFKMQIGDIEHFHNQRMMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 102 - protein, 474 aa, unidentified
  REGION 1..474: note = uncultivated organism
  REGION 1..474: note = MG119-74 Effector
MPTITRKIELYIDKGNLTDDEYKAQWQYIRQIDNTLFLAANRISSHCLLNDELEIRLKLQMPEYCKIEKLLRNSKKNNLGKKEIAELKLRRKELDAIIKKQKDEFLKTSQNSTYQLVAYEFSNIPTDILTNLNNDIVGKYGKARLDIINGIKSPSTYKRGIPIPFSVNKKLPFVFYDGEYYLEWFKGNKKVATGKELCFKLHFGKENSNNCAIVERVDESEKLGKKKGEAYVVNNSSIQLVEKGSASKIFLLLSLDIPTMKRTLDSNLVMGIDLGINYPIYYATNGNAYIKGHIGDRDSFLNERMVFQRRFKDLQRLQCTQGGRGRQKKLAPLELLREKERNWVRTKNHIFSREIIKCALKVGAGTIHLERLKNIGKDKSGNIEESKKYILRNWSYHELQEMIEYKAAMEGITVKYVNPAYTSQTCSCCGNKGERISQSVFKCLCTECSEYDKEVNADYNAARNIAKSKMFIKE

SEQ ID NO: 103 - protein, 471 aa, unidentified
  REGION 1..471: note = uncultivated organism
  REGION 1..471: note = MG119-75 Effector
MPIITRKIELKIVKDGLTDEEYDQQWKYLYQINNTIYQAANRISTHCLFNDEYEMRLKLHMPRYKEIEKELKKLDSDKKTSDKEIRDRLLNERKELDEDVKNKKKDFLQCSKQNSTYQLVSKEFKQYIPSDILANLNQKIQENYNNNQKKIESGERALSTYKKGMEIPFSIRENKRLKLFIKEEGIYLKWFKEILFRLEFGKDASNNRCIVERLIESDRQQKNKGEDYVANNSSIKLVKYGKSTRIFLLLSIDIPAKKQVLDKEVVLGVDLGIKCPLYLAINKNDNFKMQIGDIEHFHNQRTMFQKRFKSLQKLICTQGGHGRKKKLEPLEKLKEKERNWVHTQNHVYSREVIKQALKQNAGTIHMESLKDFGKGKDGYVKDEYKYLLRYWSYYELQSMIEYKAKLEGIEVKYIDPAYTSQTCSYCGERGERKKQEEFVCTNPQCKRRGEKINADFNAARNIAMSKKIVER

SEQ ID NO: 104 - protein, 466 aa, unidentified
  REGION 1..466: note = uncultivated organism
  REGION 1..466: note = MG119-76 Effector
MQTVNNRKVMPKITRKIELLFDRSGLSEEECKEKWRFIYQINDNLYRVANRLVNQLYLADEIDDILRLSDQEYIALRKKLANKKLDEATRISLEEQMSQVMKRVNERRSAILQRPQQSFAYSVVTDSDTEGLTAKILDVLKQDVLSHYKADTKEVLKGEKSISNYKKGMPIPFAFNDSLRLYKEDGFFYLKWYNGIRFLLNFGRDASNNQLIVERCLGISKDEISYKTCSSSIQIKKKGNHSKIFLLLVVDVPVEQYAQKPNMVVGVDLGLNVPIYAASNSTLERKAIGSREAFLNQRGAFQRRFRALQRLQTTKGGRGRLHKLEPLERVREAERNWVRTQNHLFSREVINFAIDVGASTIQMEKLANFGRDAQGEVREDKKYVLRNWSYFELQNLIEYKAKRAGIKVKYINPAFTSQTCSECGQLGERDSIHFKCTNPDCPNCGKDIHADYNGARNIAKSKDYIK

SEQ ID NO: 105 - protein, 467 aa, unidentified
  REGION 1..467: note = uncultivated organism
  REGION 1..467: note = MG119-77 Effector
MPKITRKIEMTLCTEGLSDEQRKEQWGLLYHINDNLYKAANNISTKLYLDEHVSSMVRMKHADYLSLLKELAKAEKKSPDEDLIAELREKLAAAEQEMTDQELAICKYATEMSTQTLAYKFATEIEINVFGQILACLKQAAQSNFKSDAKDVKRGERAIRNYKKGMPIPFPWNDNIRIDADGDEFYLRWYNGLRFHLTFGKDKSNNRMIVKRCLKMDKDFEGEYKLCNSSIQMVKRDGKPKLFLLLVVNIPQEHVELNKNVVVGVDLGVNVPAYVATNITEERKAIGDREHFLNTRMQFQRRYKSLQRLKATAGGKGRTKKLEPLERLRKAEHNWVHTQNHLFSREVVNFAVQTHAATIHMEDLSGFGKDDDGNADEQKEFVLRNWSFYELQNMIAYKAAKYVIKVEKVKPAYTSKTCSWCGQLGFRQGVTFICENPACKQCGEKVHADYNAARNIANSKDIIKKNE

SEQ ID NO: 106 - protein, 463 aa, unidentified
  REGION 1..463: note = uncultivated organism
  REGION 1..463: note = MG119-78 Effector
MPTITRKIELHLCTEGLTDEQRKAQWGLLYHINDNLYKAANNVSSKLYLDEHVASMVRLKHDGYLSLLNEYEKAKKKADEELIAELRNKVAAAEKEMTEQELAICKYATEMSTDTLAYRFANEIEINVFGQILACLKQGIHSTFKKDAADVKRGERAIRNFKKGMPIPFPWSKSIRIENEGSDFYLRWYNGLRFRFDFGKDRSNNRMIVSRCLNLDPDFEGEYKLSNSSLQMVKRDGSTKLFLLMVVNIPQENVELNKKIVVGVDLGINSPAYVATNVTMEHQRIGRRETFLNARMAIQRRYKSLQKLQNTAGGRGRKKKLEPLERLKETERNWVRTQNHLFSRDVVQFAVKARAAIIHIEDLSGYGKDDDGNADEKKEFVLRNWSYYELQTMIKYKAAKYGIKVEKIRPAYTSRTCSWCGHEGFRKGEIFICENPECEKCGEKENADYNAARNIANSKDIVK

SEQ ID NO: 107 - protein, 475 aa, unidentified
  REGION 1..475: note = uncultivated organism
  REGION 1..475: note = MG119-79 Effector
MPIITRKIELKIVKDGLTDEEYDQQWKYLYQINNTIYLAANRISTHCLFNDEYEMRLKLHMPRYKEIEKELKKLDSDKKTSDKEIRDRLLNERKELDEDVKNKKKDFFQCSKQNSTYQLASKEFLKYIPAEILTDLNRYVQNNHNNNKKKVKSGERALSTYKKGMGIPFSIKPQSGLRLFVKEEGIYLKWFKGILFRLEFGKDTSNNRCIVERLIESDKQQKKNKGEDYVANNSSIKLIKKGNDKSTRIFLLLSIDIPAKKQVLDKEVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKHNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIVKDN

SEQ ID NO: 108 - protein, 468 aa, unidentified
  REGION 1..468: note = uncultivated organism
  REGION 1..468: note = MG119-80 Effector
MPTITRKIELNLCTEGLADEEQKAQWNLLYHINDNLYKAANNISSKLYLDDHVSSMVKLKHAEYLTLVRALEKAQKQKTPDETVIEDLRRQVAAAEKDMTDQELAICNYATEMSTQSLSYRFATEIETNIFAQILDCLKQGVYATFNSDAKDVKRGERAIRNYKKGMPIPFPWNKSLKIEHKDEEFYLLWYNGLRFHLNFGKDRSNNRLIVQRCMKMDKDYEGDYKMCNSSIQFVKREGKPKFFLLLVVNIPQEHVELDKKIVVGVDLGINSPAYVATNVTMERQQIGRRETFLNGRMSFQRRYKSLQRLQTTAGGRGRKKKLEPLERLRNAEHNWVHTQNHLFSREVVQFAVKARAATIHMEDLSGFGKDNDGNADERKEFVLRNWSYYELQTMISYKAQKYGIKVEKIRPAYTSRTCSWCGHEGFRKGEIFICENPACEKCGEKENADYNAARNIANSKDIIKNHG

SEQ ID NO: 109 - protein, 494 aa, unidentified
  REGION 1..494: note = uncultivated organism
  REGION 1..494: note = MG119-81 Effector
MPTITRKIELKIVKDRLTDEKERLTDEKYDQQWKYLYQINNTIYQAANRISTHCLFNDEYEMRLRLHMPRYKEIDEGLENIKTELEKLNTKKKASDKEKRDRLLNEQRKLVDERKELDKDVKNKKKDFLQCSKQNSTYQLVSKEFLKYIPAEILTDLNRYVQNNHSNNKNKVKSGERALSTYKKGMGIPFSIKSESGLRLFVKEEGIYLKWFKGILFKLEFGKDSSNNRCIVERLIESYKQQKKKGEDYVANNSSIKLIKKGNDKSTRIFLLLSIDIPAKKQVLDKDVVLGVDLGIKCPLYLAINKNDNFKMQIGDIEHFHNQRTMFQKRFKSLQKLMCTQGGHGRKKKLEPLEKLKEKERNWVHTQNHVYSREVIKQALKQNAGTIHMESLKDFGKGKEGYVKDEYKYLLRYWSYYELQSMIEYKAKLEGIEVKYIDPAYTSQTCSYCGERGERKKQEEFVCTNPQCKRRGEKINADFNAARNIAMSKKIAER

SEQ ID NO: 110 - protein, 454 aa, unidentified
  REGION 1..454: note = uncultivated organism
  REGION 1..454: note = MG119-83 Effector
MPTITRKIELLLDRTGLSKEEVNARWNILHQINDNLYRVANNLINKLYLTDEIEDILRLNNQEYIDLRKQLGKKGLEDEKKAELEERMQEVYAEMNQQRSKILQRPRQSFAYSAVSEDGTEIINAKILDTLKQNVLSHYNADTKEVHRGEKSISNYKKGMPIPFSFDKSVRLYERNGEFFLKWYRDIQFILFFGRDASNNQLIVERCLGISGDGITYKMCSSSIQFKGKKIFLLLVIDVPRETFTPKKDMVVGVDLGLNVPIYAATNLTPERKAIGNRETFLNQRIAFQRRFKALQRLQLTQGGRGRSHKLAPLERLREAERNWVRTQNHLFSKGVIEFAKQVGASTINMERLTHFGKDKEGEVTEEKKYVLRNWSYFELQNLIEYKAKMANIKVRYVSPAYTSQTCSECGQTGERDSIHFKCTNPECKNFGKDIHADYNGAKNIAKSTNVVKE

SEQ ID NO: 111 - protein, 450 aa, unidentified
  REGION 1..450: note = uncultivated organism
  REGION 1..450: note = MG119-84 Effector
MKVTRFQLICCTTDKDGNEIDYKDIFHLLWKLQDETRSIKNKAIQLLWEWYNFSSEYKKQYGDYPSNEDTLGYKGGIRSYLYDRLKDDSSMNTANYSCTLDQSRSQFQDGLKDYLKGDRSIIEYKNNQPLELHNKSIVLERTNDEYIVKLSILSKDGAAKYNMPQRIIFKGDVRDKSTRDILNRCISGDYKVCGSKLIYIKKKKGGGDKNKSGKWYLNLGYSFESEAKALDEDKILGIDLGIVKPFVASVYGDRSRLWVDGGEDSEIEVFRRRCEARRKALLRQRKICGDGSIGHGYKTRTAPANALEDKIARFRDTINHKYSRAIVDYAVKQNCGIIQMENLKGITDGEQPRYLKNWSYFDLKTKIENKAKEKGIKVVYIEPQYTTLRCSKCGYIDKENNPNQATFICQSCGFKANADYNASQNIAIKDIDKIIKAELKKEEKEKAKNK

SEQ ID NO: 112 - protein, 408 aa, unidentified
  REGION 1..408: note = uncultivated organism
  REGION 1..408: note = MG119-85 Effector
MITVRKIKLSIRESDEESRKQKYKFIRDAQYAQYRALNLAMGVLSSEYLKSNKDTNKEDYKNAIKSLTNSNPIFSSIQFGRGVDTLSLVTQRVKKDFKISIKNGLARGERNITSYRRTHPLMTRGRSLRFYYEGDDIVIRWVNKIEFEVVTGSNKMKENIVELRRTLHKVINKEYDVKESSLMFDKNNNLILNLTLDIPNELSYQPIEGRTLGVDLGIATPAYVCLSDDTYVRKGIGSIEDFLKVRTQLQKRRRVLNQSLVMAKGGKGRKKKLKALESFKEKERNFAKTYNHQLSHEIVKFAKNHKCEFINLEKLTKEGFNNRILRNWSYYELQSMIEYKAEREGIKVRYINPAYTSQKCSKCGHVDKANRQTQSRFKCVECDFELHADHNASINIARSEEFVQGTGS

SEQ ID NO: 113 - protein, 449 aa, unidentified
  REGION 1..449: note = uncultivated organism
  REGION 1..449: note = MG119-86 Effector
MTTKVIKYEIIKPIDIDWKLAGEIFNKLQYQSYLLANKTIQMFWDFQNLSFTYKERFGKYLNMKDIPNKYTSIEGDISSQLRNQFINLPSGTFDSVVIKAKNKWNQHKLEILKGSESMVNFKRSFPICLRDRQIKIIKNDENTYILRLSLLSKEYKTELELKKGQIDFLIKAYDNTQKVILERLINNEYKIGASEIFKIKKGKTKWFINITYTFQQEENLNLDKNNIMGIDLGVCNPAYIAFNNSLNRYKIEGNEIIEFRRRIEKRRNELLKQGKYCGEGRIGHGRNTRIKPIEKLRNKVENFKDTTNHKYSKYIIDLALKNNCGVIKMEDLSGIAEGEKKATFLGMWSYYDLQTKIEYKAKEIGIEVIKINPKYTSARCNKCGYIHRSENKDIWRPKQDTFKCMNCDYGHKYFVHADYNAAKNISTPDIEEIINKQLKLQEVRVKFAE

SEQ ID NO: 114 - protein, 438 aa, unidentified
  REGION 1..438: note = uncultivated organism
  REGION 1..438: note = MG119-87 Effector
MNTTKVSRYQLLFDDENNELTFKQSNEVLFQLQKEMMQMANRMVQLYWEFSGYQSDYKKQFDAYPDKEKLLEDLKYSNIRSYIYDVLSKEFNKNNTGNVATLQQLVDKRYKQNRKEVQQGKRSIDSYKATIPISLHKGSIRLLKENGDYYAWLSLLSNEYKKELGIKKGRMRFKLLMGKSYSSMQILDSILSGEFHHTSSSLGYRNGKWFLSLGYQFEAKEPKLRESRVMGIDLGVVKPLVIAYNDMPLHHEIDSNEIQVFRDQMHARRRALGRQTKYCAESNIGHGVKTRTKALEKLKQAEANFRDRINHQYSRYVVNLAVKYGCKTIQMEDLTGISKKDTFLKNWPYYDLQTKIEYKARENGIDVVKINPRFTSQRCSKCGFISPENRPDQATFQCKKCGFGINADRNAATNIAQPGIEKIIQETIKQMSAKGKSS

SEQ ID NO: 115 - protein, 431 aa, unidentified
  REGION 1..431: note = uncultivated organism
  REGION 1..431: note = MG119-88 Effector
MNTTKVSRYQLLFDDEHNELSFQQANEVLFQLQREMMQIANRTVQLYWEFSGYQSDYKKRFDAYPDKEQLLEDLKYSNLRSYTYAVIAKEFYKNNTGNVSLLQQNVDKRYKQNRKDVQMGKRSIDSYKATIPIALQRKSILISKEGANFCVRISLLSNAYRKELGVKNGTLSFKLLFGKSYSAEQILEDIFAGKFKHTSSSISYRKGKWFLNLGYNFESEEPELRPSRVMGIDLGVVKPLVIAYNDTPVHQEIDSNEIQAFRNQMLARRRALGRQTKHCAESNIGHGVKTRTKALEKLKQTEANFRDRINHQYSRYVVDLAVKYKCKTIQMEDLSGISKDDIFLKNWPYYDLQTKIEYKAKEKGIDVVKINPKFTSQRCSKCGYISPENRPDQATFRCKKCSFTLNADWNAALNIAQPGIEEIIRETIKQK

SEQ ID NO: 116 - protein, 442 aa, unidentified
  REGION 1..442: note = uncultivated organism
  REGION 1..442: note = MG119-89 Effector
MTTKVMKYEIIKPINIDWELAGEIFNKLQYQSYLLANKTIQMFWDFQNLSFTYKERFGKYLNMKDIPNKYNSVEGDISNQLRNQFINLPSGTFDSVVIKAKQKWNQHKLEILKGTESMINFKRSFPICLRDRQIKIIKNDENTYILRLSLLSKEYKTELELKKGQIDFLIKAYDNTQKVILERLLNNEYKIGASEIFKIKKGKTKWFINITYSFEQENKINLDKNNMMGIDLGVCNPAYMAFNNSLDRYKIEGSEVIEFRRRIEKRRNELLKQGKYCGEGRCGHGRTTKLKPIEKLRGKVENFKNTTNHKYSKYIIDLALKNNCGIIQMEDLTGIADGEKKATFLGGWTYYDLQTKIEYKAKELGIEVIKIKPKNTSARCNKCGHIHRNEDKDIWRPKQDTFKCMNCDYGHKFFVHADYNAAKNISTLGIEKIIEKQLKNKK

SEQ ID NO: 117 - protein, 431 aa, unidentified
  REGION 1..431: note = uncultivated organism
  REGION 1..431: note = MG119-90 Effector
MNTTKVSRYQLLFDDEHNELSFQQANEVLFQLQREMMQIANRTVQLYWEFSGYQSDYKKRFDAYPDKEQLLEDLKYSNLRSYTYAVIAKEFYKNNTGNVATLLQNVDKRYKQNQKDVRLGKRSIDSYKATIPISLHRKSIQISKSKEDYDIRISLLSNVYRKELGVKSGLIYFKLITGKSYSSKQILESILSGKFHYTASSISYRKGKWFLNLGYNFESEEPELRPSRVMGIDLGVVKPLVIAYNDTPVHQEIDSNEIQAFRDQMLARRRALGRQTKHCAESNIGHGVKTRTKALEKLKQTEANFRDRINHQYSRYVVDLAVKYKCKTIQMEDLSGISKDDIFLKNWPYYDLQTKIEYKAKEKGIDVVKINPKFTSQRCSKCGYISPENRPDQATFQCKKCGFSLNADWNAARNIAQPGIEEIIRETIKQK

SEQ ID NO: 118 - protein, 444 aa, unidentified
  REGION 1..444: note = uncultivated organism
  REGION 1..444: note = MG119-91 Effector
MRIAYLIQQKPNLIQSGEIMTKVTRIYLISKQENVDYKKVCSVLWDLQRQTREIKNKTIQLCWEWLGFSSDYNKRTGEYPKEKDVLNYTLDGFIYDHFKKGYDLYSSNASCVTREASSKFKNAKAEILRGDRSILSYKSNQPLELHNKAISIYNENDKFYVKLKLLNKAGKEKHGVDGDFLFEMKVKEKSIRTIVERCIDGIYKISGSKLIYDNKKKMWCLNLSYSFKSTENKALDKDKILGVDLGVVHPICASVHGDLNRLIIKGGEIEIFRKRVEARRLQLLEQAKYCGDGRIGHGRHTRTLPAFKVEHKIEQFRNTANHKYSRALIDFAVKNGCGTIQMKNLKGVTTDNLFLKNWSYYDLQTKIEYKANEAGIKVVYIEPQFTSQRCSKCGFIHTENRPEQAVFKCQKCGYSANADFNASQNISIKQIDKIISETISANME

SEQ ID NO: 119 - protein, 426 aa, unidentified
  REGION 1..426: note = uncultivated organism
  REGION 1..426: note = MG119-92 Effector
MGKTDNKEEKIFRASNKVKITIVEDDPDERNRKYKLIHDEMYKQYLALNMCMSLLATQNTLRMYNSGAENRLLSQIAKLDKKISEKQQIADKDANTEKEQTRKQKAIEDIAKLEEEKSKLREEFDKKAANRTDIDVKFHEQYTSDLYQIIRNEFDFASSATIASVVRKVKDDFKNSFSMGYAKGERSLISYKRSFPLMIRGNEDACYDNSKRKWLDFYYDGDDILIRWLDGIIFKAILGNNPNRNMEIKHTLHKIITGEYRVSQSSIEFDDKGHMYMNVNYRFKKNSKTEIIPGRTLGVDLGIATPAYVCLSDTTYVRSGIGSANDFFRIRQQMKSRRKSLYKSLSLVKGGKGRKKKLQAINSLRDKERNFAKTYNHQLSNKIVKFAKDNGCEFINLEHLTKNGFEDAILSSWSYYELQNMIEYKA

SEQ ID NO: 120 - protein, 452 aa, unidentified
  REGION 1..452: note = uncultivated organism
  REGION 1..452: note = MG119-93 Effector
MSVKLTKVMKYQIIKPIDIEWELVGKIFNELQYQSWKLANKTIQLFWDFQNLNFTYKERFGEYLKIKDLPNKYTSIEGDISSQLRNQLINLHSGTFDSVIIKAKDKWNQHKQKILKGEESIVNFKKNFPICLRNRQIQIIKNDNNTYTLRLSLLSNEYKKELELKTGQIDFVIKAHDNTQKVILERLINKEYKINASEIFKIKKGKTKWFINLTYSFEQDDKINLDKNNIMGVDLGICTSAYMAFNNSQNRYYIGGSEVIEFRRRVEKRRNELLKQGKYCGEGRIGHGRTTRIKPIEKLRGRVENFKNTTNHKYSKYIINTALKNNCGIIQMEDLTGIAGGERKATFLGMWSYYDLQTKIEYKAKESGIEIKKIKPKHTSARCNKCGHIHKSEDKDIWRPKQDTFKCMNCDYGHKFFIHADYNAAKNIATPGIEEIINKQLKLQEVRVKFAE

SEQ ID NO: 121 - protein, 448 aa, unidentified
  REGION 1..448: note = uncultivated organism
  REGION 1..448: note = MG119-94 Effector
MSVKLTKVMKYQIIKPIDIEWELAGKVFNELQYQSWKLANKTIQLFWDFEKLSYSYKQRFGEYLNIKDLPNKYNSVKGDIRNQLKNQFINLPSDTLDSVLGEVESKWCQHKSKIFKGEEEMINFKKGFPINLSSKQIKIIKNDDNTYIFRLSLLSRAYKSELELKKSQIDFLVIAHDNTQKVILERILNNDYKIGVSKIFKIKKGKTKWFINLTYSFEQDNQINLDKNNIMGIDLGVCNPAYMAFNNSHNRYHIGGSEVIEFRRRVERRRNELLKQGKYCGEGRIGHGRTTRIKPIEKLRGRVENFKNTTNHKYSKYIIDTALKNNCGTIQMEDLTGIAGGERKATFLGMWSYYDLQTKIEYKAKELGIEIKKIKPKHTSARCNKCGHIHRSEDKDIWRPKQDTFKCMNCDYGHKYFIHADYNAAKNIATLDIENIIGEQMKRQNINT

SEQ ID NO: 122 - protein, 419 aa, unidentified
  REGION 1..419: note = uncultivated organism
  REGION 1..419: note = MG119-95 Effector
MTKIVKLQIIKPINQDWKLFGDILFNLRNDTRQIKNKAIQECWEYQGFSSEYKDKFEEYPKPKDVLNYSGLSGYIYNKYKLKFNRNNKANYTTIINEAVDMWKSDIKDVLRGDKSIRNYKSDLPLDLHNKSINLIKESNEYYLDLILIFNKYKKELEFKSGKFLVLIKVNDNNVKSTLDKILNNEYKISASKLIRKKNKWFINLCFSFETIASSSLSVDNIMGVDMGIIYPAYMAFNNSLDRYKIEGYEIENFRKTIESRKNKLYAQGKYCSGGRIGHGIKTRIKPIEFATKRVANFRDTTNHKYSKYIINMALKHNCGTIQIEDLKHISEDNAFLKKWSYYDLQNKITYKAKEKGIKVIKINPRYTSQRCSQCGNIDKDNRKTQKDFLCLICGFKTNADYNASRNIATKDIEDIIENS

SEQ ID NO: 123 - protein, 434 aa, unidentified
  REGION 1..434: note = uncultivated organism
  REGION 1..434: note = MG119-96 Effector
MKFSKVVKIALVSVQKDKEGNEMDYKTICQILWELQRETREIRNKSIQYCWEFSNFSSDYKKEYLEYPKPKDVLNYASIDGYVYDRLKENSSLHTNNFSTSTRDAIKKFKNLTKDILSGKCSIPEYKNNQPLDLHNKSIGIDKIKDCYYITLKLLNKKGKEKYDFGSSSIKFKALVKDNSTKTIISRLLNGEYKIGASKLIYNQKKKQWFLNLTYNFESEVTTELDEDKILGVDLGINCPFCASVYGSKKRLMIPKDEIEHFRNTVEVRRKALLKQGPFCGEGRVGHGRNTRNKPAYDIGNKVARFRDTVNHKYSRELVNYAVKNHCGVIQMEDLTGINKDSDKTFLRNWSYYDLQTKIEYKAKEAGIKVKYINPMYTTQRCCKCGHIESDNVKKHQRFACSKCGYTENADYNASQNIAMPDVEEKVAESLKVS

SEQ ID NO: 124 - protein, 439 aa, unidentified
  REGION 1..439: note = uncultivated organism
  REGION 1..439: note = MG119-97 Effector
MNKIMKYEIIKPVDCDWDLFGNILRKLKNQTRNLLNKTIQFMWEYDNFSADYKKIHNVYPNSLDILGYKGSQAVQNYAKMKLKKEWDFLFNSSNVDATLATALTRWKTDKVDILKGNKSIPSFNKNNTIDLRNSSITVIKENNSYYLNLSLISNKYKKELGRSGQFLTLIKVKDKTQKAILDRILNGEYKVSASKIIYLEKKKKWMINLGYGFEPKKQNLNKDNIMGIDMGIVYPIYIAFNNSLKRYKIEGGEIDRFRKQTEKRKNQLYRQGKYCGDGRVGHGIKTRIKPIEFTQEKVANFRNTTNHKYSKYIIDLAIKNNCGIIQMEDLSGISTNNKFLKNWSYYDLQQKIEYKAKEKGIEIKKIKPQYTSQRCSKCGYIHKDNRPKGDKGQSYFKCLECEFKTNADYNAAKNISIKDIDRIIESKCKELDITYNKNN

SEQ ID NO: 125 - protein, 429 aa, unidentified
  REGION 1..429: note = uncultivated organism
  REGION 1..429: note = MG119-98 Effector
MVKVVKIYLISEQVDEQGKDVDYNTICGVLWDLQWETREIKNKTVQLCWEWSGFSSDYYKKYGEYPKEKNLLDYTMGGFVYDKLKSKYHLYTANLSTTSQNTCGIFRTYKVDFVKGNRSVLSFKADQPLDVHKKSISIDRIDDNYFVKLKLLNKSGIQKYGIRDDFHFRMLVKDNSTKTILERCVGGDYKAAASKIIYDKKKKMWCLNLSYEFDVNTAKDLNKNRILGIDIGIVYPVVASVNGELDRFVIQGGEIETFRRRVENRKKSLLKQTKYCGDGRIGHGRNKRTEPVDIISDQIARFRNTANHKYSRAVIDYAVRKQCGTIQMENLKGITDKSDRFLKNWSYYDLQQKIEYKAKEKGINVVFINPKYTSQRCSRCGYIDSANRPKLPNQSKFLCIKCGFTENADYNASQNIALYNIEKLIDAEA

SEQ ID NO: 126 - protein, 437 aa, unidentified
  REGION 1..437: note = uncultivated organism
  REGION 1..437: note = MG119-99 Effector
MSNILSMPVKVMRYQIVKPVDADWELLGKVLRDLQDDTRKVLNKVIQLCWEWQGFSAEYKQQYGIYPKTSDFSHYKNIDGYCYKKISDFFTRLHTGNLSMTIKRGVQRWKTDLAEVIKGSKSIASFRSNCPIDIHNKSINIFNVEKEYYAKLSLLSKTYKKELSRDLGQITVLINPGDKSGKDILDRCISGEYKISSSQILQKKDTNTSKNNKWLLNLSYSFKSQKKIVDSNKVLGIDMGIIFPVYMAIHGTLIRDKIDGGEIEHFRKEVENRKIKLQRQGKYCGQGRIGHGTKTRVRPIEFAKEKIANFRNTVNHKYSRYIVDFALRNRCGIIQMEKLEGINKDKTFLKSWPYYDLQQKIIYKANEAGITGKFIDPRYTSQRCSKCGYIDEENRPEQKTFICKSCGFETNADYNAALNIANPDIENLIKKELSREA

SEQ ID NO: 127 - protein, 434 aa, unidentified
  REGION 1..434: note = uncultivated organism
  REGION 1..434: note = MG119-100 Effector
MTKVVRVYLIIEQKDKNGDIVEYTKINKLLWDLQKQTRIIKNKAIQYYWEYKNFSRDYCEKNGNYPSVEEILTYKTVDGYINNRLKIDNDLCAINRSSTIKHAIAEYKNAESDIKDGTRSIINYKSDQPLDLHNTSIHIEYIKEEYYLFANMVSREYAKANNFENARIGFKLMLQNNKSAQTIIDRCLNGDYKISESKIIYDRKKKQWCINLAYSFSPSNVQQLDYNKILGVDLGITYPLCASVYGEYNRLAIHKGEIENFRNKVEARRYSMLRQGKNCGDGRIGHGIKCRNKPAYNIEDKIARFRDTTNHKYSRALIEYAIKNNCGTIQMENLAGITDKAERFLKNWTYYDLQTKIQYKAKECGIKIQIINPQYTSQRCSRCGYIHSDNRKTQANFLCLKCGFAANADYNASQNISIKDIDKIIKKELENMRT

SEQ ID NO: 128 - protein, 433 aa, unidentified
  REGION 1..433: note = uncultivated organism
  REGION 1..433: note = MG119-101 Effector
RYQIIKPLDCDWKQFGQILRDINYETRQILNKTIQFAWEYEGFSSDYKKEFDKYPKTKDFLNYASVHGYAYNKLKTEYIKLNSGNLAQTIKRATDKFKSDRLDILKGNKSIPHYKKDCPMDVVKGSIQLYYEDKSYMANLSLISNTFKKELERKSGQFLVLLKVGDNTQKVILDRIINGEYSLSASQIINRKNKWFLNLTYQFETEKKELNNNRILGIDLGIVNVATMQIYDAEKNKYDWLKYNQCVLDGKELIHFRQKIEARKRSLQKQSKVAGGGRVGHGYNTRMKHLLTSNDKIAKFRDTYNHKVSKYIIEFALKNECGIIQMENLAGFKGEGCNLLENWSYYDLQNKIQYKAKEYGIETVFINPKCTSQRCHVCGNIDKNNRDIKKNQAKFKCTKCGYEGNADINAARNISIQHIEEIIADYIENNKVS

SEQ ID NO: 129 - protein, 447 aa, unidentified
  REGION 1..447: note = uncultivated organism
  REGION 1..447: note = MG119-102 Effector
MIGCVEVVKSLVKVVKMYLKSEQMNEQDEKIDYRTINSMLWDLQKETRDIKNKTVQLCWEWNNFSSDYYKKYGEYPKEKDILNYSMGGFVYDKLKSGYNLYSANLTMSSRETIKQFNSYKKDYITGVRSISSFKSNQPLDVHNDAIRLCRDNDDFYVYLSLLNKMGAQKYGIKGSFRFKILVRDNSTYTILDRCINGDYKVAASKLIYDKKKKMWCLNLSYKFETKKENNLDKNKILGIDMGIAAPIVASVYGDLDRFVIHGGEIEAFRRRTEARRRSLLQQTKYCGDGRVGHGVGKRMEPANNIKDKIARFRNTTNHKYSRGVVAYAIKKGCGIIQMEDLKGISDKADKFLKDWSYYDLQQKIEYKAKEASIVVSYVNPQYTSQRCSKCGYIDSQNRPKEPDQSTFKCQNCGYSANADYNASQNIAIRNIEKEISKTIENNSANNK

SEQ ID NO: 130 - protein, 450 aa, unidentified
  REGION 1..450: note = uncultivated organism
  REGION 1..450: note = MG119-103 Effector
MSITCKIKGENMGIKNTKVMRFQIIKPLDCEWDLLGQVLREIQYETRQILNKSIQLSWEYNGFSNDYKKIHGDYPKSKDILNYSNIFGYAYNKLKQEYIKLNTGNLSQTIKRGTDKFKSDMADILKGNKSIPNFKKNCPIDLKSKSIQIYKEEDSYMANISLISNNYKKELERNSGQFLVLLKAGDGTQKAILDKILNGEYKKCASQIIRHKNKWFLNLTYQFVSEKKKLNSNKILGIDLGIVNVVAMQIYNAEKDKYEWIKYNQAMISGEELIHFRQKMEARRRSLSKQSKVAGKGRVGRGYKTRMKPLLNISDKIARFRDTYNHKISRYIVDFALRNECGIIQMEDLAGFKGKGNSLLENWSYFDLQEKVEYKASEHGIEVKFVNPKYTSQRCSRCGYINEENRKRQDRFRCVECDYEANADINAARNIAIPNIDDIIKESLTIEITA

SEQ ID NO: 131 - protein, 478 aa, unidentified
  REGION 1..478: note = uncultivated organism
  REGION 1..478: note = MG119-104 Effector
MSKKEIRSVQKIKVTIVGDTPEEREAKFKIVRDEMYNQWRALNLCMSLLATQNTLQQYNSGAENRLKAQLKKLDANIDKQQKIIDNPKTKEDKLAKAIAMIKTLNDEKTKLEQDYKDKESYRTDIDVKFNEMYVKELYQIITKEFDFFASNTASCVVQKVKNDFGNSLAEYSRGERSLINYKRSFPLMLRGNADSEYESGTLKGLKFYYKDEGIYIRWCGGIEFKVVEGCNLERNLQYKHTLHKLANGEYKVSQSSLQFDKNNHLILNCNYRYNKEVDVDCIENRVLGVDLGIAIPAYVCLSDDSFIRQGFGSVNDFVRVREQFSKRRRVLQQSLTMVKGGRGRKKKLKALDRFESKERNFAKTYNHQLSAKIVKFAKENKCQFIHLEKLTKEGFNSKLLGKWSYYQLQQMIVYKAERVGIGVRFVNPAYTSQKCSKCGHIDKENRKTQAKFECIKCGLRLNADHNASINIARSDEIV

SEQ ID NO: 132 - protein, 443 aa, unidentified
  REGION 1..443: note = uncultivated organism
  REGION 1..443: note = MG119-105 Effector
MVTKVLRIEIKRVINTDKSMFNKVMHDVRYQCWKFANKAITMLWDYQQFDFSYKERFGEYFLKEQDKLPNGYKWVGSDILNVLKKDISMLESNCKDAMIRMVEQKWNNDLKDVLTGNKSIATFKRSLPVELHNKQFMDSKKNVRIFSENGVYSTVILLLSKRVAREMGMSDGKFHLELLVKDNYQKAIVDRVINGEYKLSIIKMQYNEDKKKWFLLLAYTFEAQKKQLDENRVLGIDLGLKIPAMLAISDNPFFCRVIGNSDEIKMFEKQVVNRKKRLQKARVWASEGSIGHGTKTRIKPLNNIGKKISNYKKTMNNCWSREIINVALRENCGTIHMENLRGISGRSKFLKSWTYYQLQQMIINKASEHGIKVIKIDPSFTSQRCYECGYINKGNRKTQEEFKCVICGYKCNADLNAARNIAIKDIDKVILNQLELQKNNLNI

SEQ ID NO: 133 - protein, 404 aa, unidentified
  REGION 1..404: note = uncultivated organism
  REGION 1..404: note = MG119-106 Effector
MKTVRKLKLTIIGNEEERNAGYQFIRDSQYAQYQALNISVAYLGALYLEYKDFNNEEYKSRKRNFKFNKSNPKFEGISFGKGVDTLSLISQYVKRELSNTPLKQLARGERSLTTFKRTFPLMVTGRSLKFNYDKENNVRIKWVNKISFKVVLGSGKIKENKIELQHTLNKIINGEYKVGQSSLYFDKNNNLILNLTLDIPEKDTDEVVKDRVLGVDLGIKYPAYMCLNDDTYKRESLGCAEDFIRVREQMQYRRKRLQHSLKLTNGGKGRNKKLQALNRLRDKEKDFATTYNHAISKKIVEFAKKHKCEYIHLEKLTKDGFPNTILRNWSYYQLQSYVEYKAKREGIKVRFVDPAYTSQTCSRCGHIDKDNRQTQEDFKCIKCGFELNADHNAAINIARSNNFI

SEQ ID NO: 134 - protein, 437 aa, unidentified
  REGION 1..437: note = uncultivated organism
  REGION 1..437: note = MG119-107 Effector
MKISKIVKLRLICDQTDKDGNPVEYKTICQILFDLQRQTRELKNKTIQLCWEWNQFASDYYKKHEEYPKDKEILNYSLSGYIDNKLKIDNDLYSGNCSTTIRTACKEFNNAKLEILKGQKSIISYKQNQPLEIHNKAISLEYSDNHEWFINLALLKTDAVRKYNFSSSKMRFKIIVKDNSTKTILERCLDGIYHISASELMYSQKNKAWFLNLTYSFETSNETKLDEEKILGIDLGVACPVCASVYGSKARLKIPPNEIEAFRRKTEQRKIALLRQGKFCGDGRIGHGRKKRTQPVDKIGNSIARFRDTVNHKYSREVINFAIKMNCGVIQMENLTGITENANRFLKNWTYYDLQTKIKNKAQEAGIKVVLINPEYTSQRCSKCGYIDSENRKTQAQFICLKCGFEENADYNASQNIALKDIEKIIKKTKSKSANGK

SEQ ID NO: 135 - protein, 455 aa, unidentified
  REGION 1..455: note = uncultivated organism
  REGION 1..455: note = MG119-108 Effector
MTTKVVKIEIIKPINDTWDVLGQVLRDIQYQTWKLANKAIQMFWDFSNFNYSYQQRFGESLLGKGKSLPNGYKNVGSDILNECKEFTDKLNSNSKDAIIKMVTDKWNADFNDIINGRKSIANFKRDLPIELHNKQFMNSKKQLMLYVENGDYYIEINLISKQYAKELGKKSTAFKLLLAVKDNYQKAIVDRLISGEYKLGMSKITKTKKGKTKWYLNLVYSFTEKKDDSLDLNKVMGVDLGVNIPAALAIYGDKYFKEFVGDRDEIENFRKQIEARKRKLQRQGKWCGEGRIGHGIKTRIKPLEKLAGKIANFKDTKNHCWSRYIVDVAVKNKCGVIQMEDLSGITEENTFLKNWTYDDLQQKIKYKAEEKGIKVIYIDPAYTSARCSKCGHIHRSYEKKDWRPSQDQFICQVCGHKENADINAARNIAIPNIDKIIKEQLEKQEREQRNQKYVG

SEQ ID NO: 136 - protein, 434 aa, unidentified
  REGION 1..434: note = uncultivated organism
  REGION 1..434: note = MG119-109 Effector
MVKVVKIYLISEQTDKQGNVIDYKDIFKCLWDLQRETREIKNKAVQLCWEWSGYSSEYNRVHGIYPKEKEVLNYTLGGFLYDNLKNGYNLYSGNLSSTLQLVEKAYKASKLDFMRGDKSILSYKANQPLDIHKKAMRLEKTGNEFLLYLSILNKAGIQKYGIDSAFKFKLIIKDNATLSIVERCVDGTYQVATSKLIYDKKKKMWCINLSYNSECKHNEVVDKNRILGIDIGVACPLVASVYDDWDRCTILGGEIEEFRRRVEARKKSISKQTKYCGDGRIGHGINKRTEAVNKIGDKIACFRNTANHKYSRTVVDFAVKKQCGTIQMEDLQGITDETDRFLKNWPYYDLQTKIENKAKEEGIKVVYISPQYTSQRCSKCGYISKENRPEQAKFECKECGFKENADYNASQNIAIKNIDKIIEKDMSKDGAKGK

SEQ ID NO: 137 - protein, 426 aa, unidentified
  REGION 1..426: note = uncultivated organism
  REGION 1..426: note = MG119-110 Effector
MVKVVRLRIIKPIEADWDMLGKVLRECSYEAMLLRNRAMREYYLKNAEAYAYYKQTGKNMEYVLNDKTYTSLESYIYALFTDTCEYSNSELKSSVLRDVAKKWKGVSKDVTTGKMSLPTYKQGCPIEISGRAMTLEYDSETNNFYLKLRLLSKEASKKLELKNGVFKVLVAVEDNSKRTILERCVDGVYSLAGSKIMQVKKKWVINLSYSFEPENNSTLDKDTILGVDMGIVYPVYMAVNNSLAREKIVGGEIEAFRKRVVKRKNELYQQSKYCGDGRINHGTKKRIEPIVKIGAKEADARDTYNHKYSRFVVQTALKHGCGTIQMEDLSGITVDTDSKFLKNWTYYDLQNKIENKAKEVGIAVVKVEPAYTSQRCHKCGFISADNRKTQSDFECLECGYKGNADYNAAKNIATKDIAKIIKETKK

SEQ ID NO: 138 - protein, 464 aa, unidentified
  REGION 1..464: note = uncultivated organism
  REGION 1..464: note = MG119-111 Effector
MKTTRKLKLTIIGDEETRKEQYKIIREEQYQQYKALNLCMTLLNTHNILNSYNTGAENKLNAQIDSIDKKIEQAKKELEKKGLKESKVSKLKETIEFLENDREKLKDEYLNSSKFRSDIDEKMKEMYIKDMYTVVQNQVNFRARDMMSLVTQRARKDFKNSLKNGMAKGERSLTNYKRDFPLMTRGERWLKFEYDKDSDDILINWIHGIKFKVLLGYKKNENSIELRHTLHKVINKEYKICDSSMQFDRNNNLILNLTLDIPDKQNNNYIEKRTLGVDLGIKYPAYVCLNDDTYIRSHIGESLELLKQREQFKDRRKRLQQQLKNVKGGKGRNKKLSALNKLSDNERNFARTYNHMISKRIVEFAKKHRCEFINLEKLTKDGFDNNILSNWSYYELQNMIEYKAKREGIEVRYIDPAYTSQKCSRCGYIDKENRQTQEKFKCLKCEFEINADHNAAINIARALD

SEQ ID NO: 139 - protein, 424 aa, unidentified
  REGION 1..424: note = uncultivated organism
  REGION 1..424: note = MG119-112 Effector
MIKQIKLEISGSKELSCKELYKLLFDLRHETFLFKNRDLQCLFDFTQFEFSFRQKYGEHYKTLHDKTPAGVTPNTEAYREGKRLVSQGKNNYLKSNSNSFDAAHRSVVRKYNSELNDVLSGKRTFSTYKKTTPIELHNKQFYTGNTKTHRFYVENNKHYIAINLVSNEYSKVLNLNGTVVTLEVLVKRNHTYSIFQRILNYEYRLAMSQIKYDEKSKKWFLILAYKFDQIKNNDLVNHRILGVDLGINIPVMLAINDNPYYRKSIGLREEIEQFNKQMIKRKKILQRTARYAGRGKVGQGRKKRTEAMKKIGHNIENFRNTKNHSYAREVVNEAIKNKCGVIQIENLKGILKLNAFLKDWTYYDLMKKIKSKAEEVGIKVILINPQYTSSRCSSCGTIHFNKSKSMYRPEQDRFICQNPSCNFS

SEQ ID NO: 140 - protein, 439 aa, unidentified
  REGION 1..439: note = uncultivated organism
  REGION 1..439: note = MG119-113 Effector
MITKTMKYEIKYNKELYSLLNDIQYVIYRLKNKATSMAWDWQQFSFGYNERFGEYPKEKDIMGKTLSPDIYNVIKDEFGDFVSSSTVDVAIQDAVKKFKNDSKEINRGERSIANYKRDGAFPLRKTQVKKLEKVNSKTFTAKFSLLSNEGKKQRELKSGQIDVTLKTGGGAAVILERVMNGEYKMCDSQIGKTKNKFYFIMTYQLEKDDIVILDENKIMGIDVGYNIPAVIATNFDDWYRQFVGSGKEIRDFETKTLARKRRIQQSRKWCGKGSRGHGTKTRVKAVTKINDSVSNFKQTKNHNWSKYIVDEALKMGCGTIQMEDLSGINEKNTFLKTWSYYQLQQYIEYKAKSYGIKVVKIKPNHTSARCNRCGYIHSGKDADKWRPASENKSMFYCQNCNHKSNADHNAAKNIAMKDIEEIIKHQLYIQENVKEKLKV

SEQ ID NO: 141 - protein, 467 aa, unidentified
  REGION 1..467: note = uncultivated organism
  REGION 1..467: note = MG119-114 Effector
MITVRKLKLTIVGDEETRNQQYKLIRDEQYQQYRALNLCMSLLSTYNILNNWNSGAENKLNSQIEKLNKKVEKNKNDLKKDNLKENRIKKINESIQTLTKEKEKLQQEYLSSSEYRSDIDKKVKEMYIDDLYTVVQSQVNFKSKDMMSLVTQRAKKDFTTALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRTLGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKYGFPNMILSKWSYYELQNMIEYKAEREGISVKYVDPAYTSQTCSKCGHVDKENRTSQEKFKCIECGFELNADHNAAINIARSNNYVK

SEQ ID NO: 142 - protein, 468 aa, unidentified
  REGION 1..468: note = uncultivated organism
  REGION 1..468: note = MG119-115 Effector
MTTTKKKEKENNNNNNAPKIPTQTVRFGLLFEENAEFSYKDLRSSLWSIQRDIREFKNSAASQYFDYMTRREKIKKETGKYPADADLLNGRKIRNHLYHLAADIIPSLQTGNTTAVSESAYSRFSTDYQDVLAGRKSLASFKQDQPINIKKESIKFISTGGKLYMKLSVFSTEGAKKLGLAGGQVTFEVWRKSKSGHAIINRCISGEYSYGSGMLIYNKDKRMWECNLTYTFAPKKNDLNPDTICGIDIGFIHPIYAAVSDSRERFWLPGSEIDKFRRGVEARKRDFSRSRKFAGEGSVGHGYAKRMEPVNDIKRKIANFRETKNHQYSKKFVEFAVKNRCGVIQIRDLSGITDKTKEAFLKNWSYDDLKDKITYKAKAEGITVISDILPHYTSQRCFKCGHISKDNTNKRKFVCLSCGHEEHVDYNAAQNIAIKGIDEIIERDCIAQGIPYPPENKKEKEMEKEEDD

SEQ ID NO: 143 - protein, 467 aa, unidentified
  REGION 1..467: note = uncultivated organism
  REGION 1..467: note = MG119-116 Effector
MITTRALKLTIVGDEETRNKQYKYIRDEQYEQYKALNLCMSLLNTHYILNSYNSGAENKLKSQLDKLEKNIEKNNLELAKDNIKESKKEKLINQNIKYKEDLKKLQEEFNKASTYRSNIDIDINDMYIKDLYNVVQNQVSFKNKDLMSLVTQKAKKDFKTCLKNGLARGERSLTNYKRSYPLMTRGNRYLKFRYDEESDDIYIDWIQGIIFKVVLGHRKNENTIELRHTLHKVISGEYKICDSTMNFDKANNLILNLVIDIPINENNNYIEGRTLGVDLGIKYPAYICLSDDTYKRMAIGCAEDFIRVREQIRGRRFRLQKQLKMVKGGKGRDKKLNALDRIRDKERNFVNTYNHMVSKNIVDFAFKNRCQYIHLENLTKDGFENAILSKWSYYELKKMIEYKAERKGIKVRYVNPAFTSQTCSKCGHVDKGNRQTQEKFKCTKCGFELNADHNASINIARSKDIIK

SEQ ID NO: 144 - protein, 341 aa, unidentified
  REGION 1..341: note = uncultivated organism
  REGION 1..341: note = MG119-117 Effector
MINVRKLKLVIRETDKELRSQKYKFIRDSQYAQYRALNVAMGYISSAYLKANKDIKSEEYKETIKLLTNSNPIFNGIEVGTGIDTLSAVTRKARKDFSASLKNGLARGERNLTSYRRTYPLITRGRDLKFRYEDNDIVIKWVNKIEFTVITGSNNMKENTIELKHTLHKIINNEYKIKESSLMFDKNNNLILNLTFDIPDKEAHEIVPGRTLGVDLGIATPAYVCLSDNTYIRSGFGSANDFFRIRQQMKSRRRSLYKSLSLVKGGKGRKKKLQAINSLRDKERNFAKTYNHQLSYKIVKFAKDNGCEFINLEHLTKNGFEDAILSSWSYYELQNMIEYKA

SEQ ID NO: 145 - protein, 429 aa, unidentified
  REGION 1..429: note = uncultivated organism
  REGION 1..429: note = MG119-118 Effector
MTKVIKVYLISKVLNANGEEVKYEDVNKILWQLQHETRQLKNKIVQLCWEWNNFQSDYYKENGYYPKDKEVLNYSFAGYVYDKIKNLTILNSANFSLVSQDVIKAFKNSIDDIRKGNKSIINYKTKQPIDLHNKSISLSYDDKKFFVNLSLVNNGFAKENNLKRFRFECIVKDNSTKTILERCMDKIYKISGSKLSYDKKKYMWYISLCYSFENNNKINLDENKILGIDLGVKKALVASVYNDFDRLDISGSEITKFRNTIEKRRKELLRQTKFSGNGKIGHGRNTRCKSIDKISDNISCFRDCANHKYSRAVIDYAIKKGCGIIQMEELSGISEEAKPFLKNWSYFDLQNKIKYKANEVGIKVITVSPSYTSQRCSKCGNIDESNRITQASFKCTSCGYSANADYNASQNLAIRDIDKIISETRANMK

SEQ ID NO: 146 - protein, 426 aa, unidentified
  REGION 1..426: note = uncultivated organism
  REGION 1..426: note = MG119-119 Effector
MTKVVKVYLISKVLNVNGEEVKYKDVNEILQQLQNETRQLKNKIVQLCWEWNNFQSDYCEKNGSYPKDKEVLNYTLSGYIYDKLKDLTIHNTNNLSSIIQDATKAFKNSKADIMKGNKSIINYKANQPIDLHNKNISLSYDDKKFFVNLSLVNNGFAKENNLKRFRFECIVKDNSTKTILERCVDKVYKISGSKLSYDNKKKMWYLNLCYSFENNCKLELDKDRVLGVDLGIVNPIVASVYGDIDRFVIDGDEIIKFRQKTEKYRVKLLRQGKYCGDGRIGHGRKTRCKPIDRIGNRISRFRDTTNHKYSKALVEYAIKKGCGTIQFENLSGISKDKEQFLKHWSYFDLQNKIKYKANEVGINVVFVSPKYTSQRCSKCGNIDENNRITQASFKCTSCGYSANADYNASQNLAIRDIDKIISANHM

SEQ ID NO: 147 - protein, 426 aa, unidentified
  REGION 1..426: note = uncultivated organism
  REGION 1..426: note = MG119-120 Effector
MTKVIKVYLISKVLNANGEEVKYEDVNKILWQLQHETRQLKNKIVQLCWEWNNFQSDYYKENGYYPKDKEVLNYSFAGYVYDKIKNLTILNSANFSLVSQDVIKAFKNSIDDIRKGNKSIINYKTKQPIDLHNKSISLSYDDKKFFVNLSLVNNGFAKENNLKRFRFECIVKDNSTKTILERCVDKVYKISGSKLSYDNKKKMWYLNLCYSFENNCKLELDKDRVLGVDLGIVNPIVASVYGDIDRFVIDGDEIIKFRQKTEKYRVKLLRQGKYCGDGRIGHGRKTRCKPIDRIGNRISRFRDTTNHKYSKALVEYAIKKGCGTIQFENLSGISKDKEQFLKHWSYFDLQNKIKYKANEVGINVVFVSPKYTSQRCSKCGNIDENNRITQASFKCTSCGYSANADYNASQNLAIRDIDKIISANHM

SEQ ID NO: 148 - protein, 455 aa, unidentified
  REGION 1..455: note = uncultivated organism
  REGION 1..455: note = MG119-121 Effector
MITVRKIKVYCDDSTFYSFLRNEQMEQNKALNMAITLLHSANTIKSIDSGAETRILKAIEKNKQVVEKLKQDLETKKTESAKEKIIEKIKLREDLIEQDEKILEEGKVFRKGLDKEFSKIYIDTNNLYQVLSSNTKISYMRTLDLAVQKAKADYSNNFVNIVTGQQSLQNYKSDIPLMIDKKNINLFKEDNNYKIRIMLGYELNVVTGNKQNNELITTLDKIINGEYSICQSSLMFDKANRVIFNLCLDIPDKKGYEPVKDRVLGVDLGIKYPVYMSIPNTYYKKSLGSINDFLRVRQQMQDRRKKLQSELQLTKGGKGRNRKLQALDRLKEKERNFVKTYNHTLSSRIVEYAKKNKCEYINIENLTKDGFPDSILRNWSYYELQTMVTYKAKREGIIVRKVNPAYTSQKCSSCGHIDSDNRQTQEDFRCVKCGFEANADYNASINIANSTEFVR

SEQ ID NO: 149 - protein, 441 aa, unidentified
  REGION 1..441: note = uncultivated organism
  REGION 1..441: note = MG119-122 Effector
MTKVTRIPLICAVTDKDGKEIDYKDIFHLLWDLQRETRMLKNKAIQLCWEWYNFSSDYKKEHGDYPDNESILGYKGGIRSYLYDRLKGESVMYSTNISCALDSARNQFQASLKEYLRGDCSIIEYKSNQPIELYNKAILLDYHDNEYWIGLKLLSRKGEKEKSFPSCLLFKGVVNDKSTRDILNRCMDGVYKISGSKLIYDKKKKWCLNLSYSFEPEMRSLDKDKILGVNVGIYKPFMASVYGDKARLFAEGGRDSEIEVFRRKIEGRRRSILKQGKMCGDGRIGHGYKTRTAPANALEDKIARFRDTMNHKYSRAVVDYAIKNNCGIIQMEDLTGITEGEKPRYLKNWSYYDLQMKIENKAKENGIKFVKIAPQYTSQRCSKCGYIHADNRPDQPTFECLKCGYKENADYNASQNIAIKDIDKIIEASDIPKKKKKSDVA

SEQ ID NO: 150 - protein, 530 aa, unidentified
  REGION 1..530: note = uncultivated organism
  REGION 1..530: note = MG119-123 Effector
MSKKGKKDKKNKKIVLCRKLKLTILGDEQTRKEQFKLIRDDQYQQYKALNLCMDLYQANHALRKYEFKLEVDSRKELNDSIPKKEKLIEKILTLEKQIISLEEKGKLNEKDDEKLEQKKSSLNKKNDELSALEEKILKLKNNIKNSIEYQASLSEKMQEKYIDDIHTVVQNQVNLNYKDTLSLVTQKVKEDFKIALNNGLKEGKRKSTSYKRDFPLMTKGRELKFKYRDENSDDIIIKWVKGITFKVILSNKKNNDKELRHTLHKIINNEVKICDSSLQFDNKNNLILNLAMEMKVERGFTPIEGRVMGVDLGMKIPAIASFNNMNGFKKFGSIDDFLRVRKQIQARRRKLQIALTTVSGGHGRDKKIQALDRFSKSEKNFVNTYNHTISKKIIEYAKVNKCGVINLEFLSLADQEKDLFLTMSESKIKQSRFIKRNWSYFDLQTKIIDKANREGIKVLLVDPYHTSQTCSKCGHYETGQRTSQAHFKCKSCGYEANADFNASQNIAKSTQYIDKKEDSQFFKNKKSKAS

SEQ ID NO: 151 - protein, 452 aa, unidentified
  REGION 1..452: note = uncultivated organism
  REGION 1..452: note = MG91B-1 Effector
MSGEGRYILTLPLKTELYQEHILEKRFRLGEELYNKFLSYEKKKYYEMTKRRDYRDAQSLIKDLTMQINELDKQSDKSKEFKKLSLDLKKKRKEQYDAIYNIKKMFNWSEFGFGMDMKKYRGYYKKNLNSQICNNLAVRCFRAFEKMEEELIKDQYRSDEDKVNPYVHYKRKDSLRSLCGSQNTTGIRFILDDEKKVGLVKWQGLEFIIDLTKCTLYEWKALQSEICYCAVKREKIKGKWKYYIQITLKGHVPDKFNKQTGELKRQLGKGNVGLYFTSTSLTVSTEDGTKTYPLAIKNHEDKKTELLQKMDASRRATNPDNYNDDGTSKEKNEIKGWHFSKAYKKYRAELYEIFRKECEEKKLLQEVLANEVIASGDVFNCNKMDFKFLQKNLGKKIQSASPAMLKTTIERKLSYHDVSINEIGYSKLNPIFEEKNVSKKDLEEVAALIRQF

SEQ ID NO: 152 - protein, 512 aa, unidentified
  REGION 1..512: note = uncultivated organism
  REGION 1..512: note = MG91B-2 Effector
MKKSSTDTCCLTLPLKLEKWQEDRLAKRFEIARQLYNTLVHAELKRLAQVKQTAEYRENERKIRTLDWNDPGDQAKLKGLYKERRKSLKTAGFTDYSFTTDMGKYYKHFRDNIGSSVAVHGIAPQVWAAFDKLLFKKGGKKVHYKKPGEVNSVQGYSRTGKSGGREIMFRGAYIEWKGLKLPLKLSPDNAYETDMLQRRVKLVRLVRRPGKRRDRWYAQLVLEGKPAIKADPVTGKAVHPIGSGPVGLDIGPQTLAYSAAGVAGLVELADRVQNIEQEKRCLQRKMDRSRRATNPDNYAEDGTIRRGVKLTHSKSKRYRRSQQELKYLQRRQAEIRKLQHIQLANHLLSLGDHFYVEKMEWSSLTHRAKKTVISEKTGKPRSKKRFGKSIANKAPAMLIEILRQKCKSLGLPNVVEINPADIKASQYNHLTGEYVKKSLSRRWNEMQDGEHLQRDLYSAFLLQHYDSMSKALDLDSLARDYPAFVQYHHREIERLHALPKTPSSMGIRRSIA

SEQ ID NO: 153 - protein, 511 aa, unidentified
  REGION 1..511: note = uncultivated organism
  REGION 1..511: note = MG91B-3 Effector
MLVSKRSTTDTCCLTLPLRLEKWQADRLAKRFEIARQIYNTLVRAELKKLVRLERISAYKDIQQQLYSLYRDGHQNSREYKVLFQKRRSLLTEAGIREYGFKGDIAKYYKHFHENIGSSVAVHCIASQVWCAFDKYLFHNGKQVHFKRAGDIRSLKGYSVSGKSGGTEIIYRGTFIEWKGLRLPLKLDPDNRYEAEMLTKRVKYVRLLRTRGNHQDRWYAQLVLEGTPAVKHDAKTNALRHPIGHGAVGIDIGPQTIAYVAQDEAALMELANRVQNIEREKLLIQRKMDRSRRATNPDNYAVDGTIKRGVKLTRNKSKRYCTLQHRLAFIQSRQALIRKVQHTELANHLLSLGDRFYVENMEWSSLTHRASETAISEKTGRYKRKKRFGKSISNKAPATLIGILEQKCKSLGLPGVIKVPTKLCASQYNHMTDTREKKPLSQRWNDMPDGHRIQRDLYSAFLLQHVTPQEDGFDRPSLQRDYEKFVSLHDEVIRQLTLAPKTLSSMGIRRT

SEQ ID NO: 154 - protein, 512 aa, unidentified
  REGION 1..512: note = uncultivated organism
  REGION 1..512: note = MG91B-4 Effector
MPTKQKEGGNRCVLTLPLLTEPWQEHIIEKRFKIIEHLKNQLISIELRKLKKLQRTKAYKNLIKQIESVPKSERTVLYKERNKMLKDAGFSQYTFMDDMAEKKSLMQKHFESHINVQIAHKAAIDVWSSFEKFLFGTGQIVHYQKRGTLSSVASKSPKTGMFYYSDENIFKWSGGGTKPKGGIILCLRVASVKTNYEKEMVANNEIKYYRVVRKWMKTKYKYYLQITFAGKPADKHREISTGRVGIDIGTQTIAIASDKSVKLLELADKVNKNHIKKCELQRKLDRSRRYTNPDNFNPDGTVKRGTKGHKLKWNKSKHYLEVAGKIRELERKNADIRKYQHTCLANDILAMGNQIYIEPMNYKGLQKRAKETQVDEKGRYKKKKRFGKSLANKAPAMFVTILKTKAEQHGGEVYEVDIMSYRASQCDHTTGEYTKHGLNDRWRILGNGDKVQRDLYSAFLIKNSNKTHDEIDLDKCSKTYDSFKKLHDKEIENIRNEVILRKRRNISSYGIA

SEQ ID NO: 155 - protein, 534 aa, unidentified
  REGION 1..534: note = uncultivated organism
  REGION 1..534: note = MG91B-5 Effector
MGWKSKGRGGTLVKKSSTDTCCLTLPLKLEKWQSDRLEKRFEIGRQIYNTVVRAELKKLQQVERTPQYREIEQKLKEAKQEEAKELWKTRTQLLSKSGFSEYGFKSDMKYYYQHFKENIGSSVAVHGIASQVWTAFEKVLFSKDGKMVHFKKKGELSSLRGYSQAGKSGGIEIIFRGSYVEWKGLKLPLKLSPDNDYETEMLSKRVKYVRILRKEGKTKPHWYAQLVLEGKPAIKRDPASGAPKHPVGHGVVGIDIGTRTLAYSADSKVDLIELADRVQNIEQEKMLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRIIS

SEQ ID NO: 156 - protein, 511 aa, unidentified
  REGION 1..511: note = uncultivated organism
  REGION 1..511: note = MG91B-6 Effector
MKKSNTDTCCLTLPLKLEKWQEDRLAKRFEIARQIYNTMVHAELKKLRNIENSQPYRQIQKQIEALNWKNQNDKARLKSLYNDRNKLLNTIGFSEYGFKADIKYYYKHFNDNIGSSVAVHGIAPQVWAAFEKMLFHKEGKKVHYKKKDDIHSLHGYSVTGKSGGVEIIFRKTYIEWKGLKLPLKRSPDNAYETEMLGRRVKYVRLLRKPGKRKDCWYAQLSLEGKPAIKRDPESGKRRHSIGRGAVGLDIGPQTLAYSACGAADLVELADQVQNIEQMKRRLQRKLDRSRRATNPDNYADNGTIRRGVKLTHNKSNRYRRTQRELRYLQHCQAEIRKRQHIELANHLLALGDCFYVEDMAWPSLTHRAKETAISEKTGRIKRKKRFGKSVANKAPATLIGILEQKCNSLGLPGVMKVPTSLRASQYNHQSGEYTSKPLSQRWNNMPDGERIQRDLYSAFLLQHYDPKAGNFDSALLQQDYPQFVQYHHRAIQRLSTVPKTLSSMGIQRSLR

SEQ ID NO: 157 - protein, 515 aa, unidentified
  REGION 1..515: note = uncultivated organism
  REGION 1..515: note = MG91B-7 Effector
MAEETGRCVLTLPLLTEPWQEHIMEKRFSIVEHLKNQLIAMELRKLKNLQRTKAYRELAEKIEATPKEKRAPLYRERKKMLQDAGFSEYAFKHDMQSPENPMQQHFSMHITSKVAERAASDVWRAFQKYLYSNGNEVHFHKRGTLKSVACQANGSGMYYQDDTFAWHRGKTAKVKPAKDNPQGRKKRDKNKQLHIKVRPPQNAYEREMLKKEVKEIRVVRQWVKSRYKYYLQLTLVGPPVPKERKLGVGRVGIDIGTQTIAISSETSVQLRELAPGVNDNHEKKLRLQRQMDRSKRATNPENYNPDGTIRRPPKGERMRWNFSKRYKKLAGKVRELERKNAAIRKYEHTCLANEILSLGDEIYVEKMSFSGLQRRAKETKKDAKGCYLKKKRFGKSLANKAPATLMQILKTKAALTGQVAEEVNTTAFRASQFDHTSGQYRKPLLSERWKTLSGGERVQRDMYSAFLLMNSDSDKTQSDAELCSLTFENFRRLHDAEVTRIRNEHKRHLRSFGID

SEQ ID NO: 158 - protein, 527 aa, unidentified
  REGION 1..527: note = uncultivated organism
  REGION 1..527: note = MG91B-8 Effector
MNEVFVLELPLQVEKYQEDILDKRFEQLRQLYNYVQGKLLRQYRYFEQTKEFQACKTIKEKRDFFRNHPFHFNGIYGNNNELFNITFDEYGVNGFVSKMAKKQVGANMTYADLGITTTLLDRLSLAIWASWEKKMYDYHTKRISFKKHGELNIFGSRKKTNGSGKKMFSGMELNLERMEVSIKLNGKVGKKAKYITLPILHNPKHVDYEMMALKGGFDSVKVITVVRRLVRGRYKYYLQMSIEGEKPKKDRTLGKGNVGIDIGPTTVAVSGENIASIDKLASKCDSIEAEKARLGRKIDRSRRANNPQNFNEDGTIKRGVRLVWNDSKRYKVLRREMTELQRRQAAIRKQQHIDRANELLKEGDTFIVENNQISGWTRKAKETKVNEKTGKIQKKKRFGKSVANHAPSMFVTILENKVKSLGGQVVKVDTKNAASQYDFTNDSFEKHELNERSVTLSNGDTHQRDMLAAFNLQHLKYDAQEKKQYDREAMTQHYDRFCKLERAEIMRYKNKEKRDDRSTIGAGELNT

SEQ ID NO: 159 - protein, 518 aa, unidentified
  REGION 1..518: note = uncultivated organism
  REGION 1..518: note = MG91B-9 Effector
MDKEVFVIEFPLNVEKWQADILNKRYEHLRTLYNYVQGKLLRQYIYFAQMGEYKNCKTPKERREFFKTHAFHIKGISGRNNITFTEYGIHSLVTKLAQHRIGADCTYRDKGINSMILDHMAAYIWSSWNKKLYDINARRISFKNFGEINTFGVRKINGARFCGLNLHLDIMTLDIKINGKNGQQAKFISLPVRYNLKHAGYEMNALAGGFDSIKVLTVVRRMVRGRYKYYLQMTIEGVKPQKGRHLGNGRVGIDIGPTTIAVSSNSTVSIDKLAAKCDNIEQDIQRIGRKIDRSRRAMNPDNFNADGTVKRGVRLTWVKSERYKALCQEKAELQRKQAAIRKAQHIERVNTLISEGDTFIIESNPIKGWTARAKETKVSEKTGRFQKKKRWGKSVANHAPSMFVTILENKVKSLGGTVIKADVKNAASQYDFTDGTFTKHELDERGVTLSNGDTHQRDMLSAFNLQHLIYDTKELKQYDQQQMEIHYPRFCELEQQEIEKYKQRIKTDDRSTIAASRL

SEQ ID NO: 160 - protein, 524 aa, unidentified
  REGION 1..524: note = uncultivated organism
  REGION 1..524: note = MG91B-10 Effector
MNEVFVLELPLLVERYQADILNKRYEQLRQLYNYVQGKLLRQYRYFEQMKEYKACEKIKEKRDFFRAHPFHINGVLNGNKKQLDITFDKNGINGFVEKLAQKSAGPTITYADLGFTTYILDHLSASIWAAWEKKLYDFKAKRISFKKYGELDSFSSRRKVVGGKVYFSGMELHLDTMKLSIKMNGKQGDNAKFITLKMLHNPKHADYEMWALKGGVDSVKVVKVVRRLVRSQYKYYVQLTIEGEKPQKGRKLGNGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIERANALLKEGDTFIVENNQISGWTTRAKETKVNEKTGKIQKKKRFGKSVANHAPSMFVSILENKVKSLGGEVVKVDTKNAASQYDFTDNSFTKHELKERNVTLSNGDTHQRDMLAAFNLQHLKYDEAEAKQYDTEAMAANYERFCQLEQEEIQRYKNKEKKDDRSTIGAEEL

SEQ ID NO: 161 - protein, 509 aa, unidentified
  REGION 1..509: note = uncultivated organism
  REGION 1..509: note = MG91B-11 Effector
MARCTTPTCVLTLPLRLEKWQEDRLAKRFEIARKIYNALLGAELKKLRRLEQTPEYQAIKKELAALYSEKDKNADRIRALYKERNKLLKDNSFTEYGFTVDAKFYYKHFRTNIGSCVTVHCIAPQVWSAFEKYLFGNGKRIHFKRPGEIYSLRGYSSKASSGVEIIYRGDHIEWNKLHLPLKLDPGNQYEEEMLTNRVKYVRILRRPGKNRDHWYAQLTLDGLPAIKRDNETGDPVHPIGHGPVGIDIGPQTIAYSSGKEAGLLELADRVQSIEDEKCRIQRKMDRSRRATNPGNYNLDGTIKRGVPLTRNKSKRYLRLQKELASIQHQQAAIRKEQHVALANYLLTLGDRFYIEDMNWQGLARRAKEATIPEEEKPHKRKKRFGKSIANKAPASLVTILKNKCTSLGLPGVIPINTKVRASQYNHLTDSYQKKELSQRWNEMPDGRKIQRDLYSAFLLQHMNSQLDGFDRAALQRDYESFVSLHDQAILRAAAAPKLISSMGVKRTVN

SEQ ID NO: 162 - protein, 614 aa, unidentified
  REGION 1..614: note = uncultivated organism
  REGION 1..614: note = MG91B-12 Effector
MAKENFVLTLPLKVEKCHADILDKRYELLRQIYNMVQQKLVRQYIYFSQQKAFKACENFDQKREFFNKHPFHFKGIKGKNGELSDIKFPYTYESRSSSSGKRPSGGISDYVAKLWSHKVGADKRLVDFGLNASIMEELGLHIMKAWEDRLYKFKAKRVSFKEYGEINTLGCRLKGNFAGFTINLEEGFITFNTNGRHGKFADLIKLPIDFSREKRGYAEDALKGGVDSLRKLVIVRRCIRGRNKYYLQLTISGEKPQKGRTLGIGDVGIDIGPSTIATSSLQGVHFDKLAEKCDNIEHELYLINRKLDRSRRANNPDNYNHDGTIKRGVRLVWKKDKDVKRYASLKQQRKELFRRQAEIRKLQHLEMANAMLSQGDTFIVENNPIDAWVRRAKETKKTKTGKNRSKKRYGKSVANHAPAMFVTILENKVKSLGGVFYKVDPGYAATQFDFTNGVFTKHEVSERSITLSNGRTHQRDMLSAFNLQHARLEVEKAKSRDKEKKVESYDIGQMQADYPIFCRLETAEQERFKNARRNERDALYFVGALAKELRHAPQEETVANRHNGGVGRKHCNTLCPENAKITGNGLVRESVARVSEAPEECYHTGDQPIILDGL

SEQ ID NO: 163 - protein, 508 aa, unidentified
  REGION 1..508: note = uncultivated organism
  REGION 1..508: note = MG91B-13 Effector
MGKGNKNGEDDTSYCVLTMPLYPEPWQSEILETRFQIMEHLENSLTALELRKLKNVERTKKYREVMSAIKEEKDDKNRNKLWKERKKLLKDAGISQFDFIKDISSLQKHFAPHIAQKVGAAAAHSHVWSAFDKVVFGKGQRIHFHKKGDLKSITSGYFGVSMTVKNGYFIWSGGTKDEKDRKVKPITISVKIKQPETYYEKEMLQKQIKYVTIVRKWIKNRYKYYLQFMLVGNPVVKPRVIANGKRVGIDIGPSSAAIVSEKEVKLVELADKVQKNHDKKTQLQRSMDRSRRAMNPQNFNKNGTIKRGIKLIWNESNHYKEMRGQIRELERKDADIRKYQHTCLANYVMSLGTEVYVEQMNFKGLQKRAKETKYDENGRPKRKKRFGKSIANRAPAMFITILNNKLSGIAGEELHKVNTFTFKASQYDHTNDTYKKKLLKDRWAELSNGDKIQRDLYSAFLIMNSDAEMQQADLRKCEETYKRFKEQHDGLIVKMKESNHIRLSSFGF

SEQ ID NO: 164 - protein, 508 aa, unidentified
  REGION 1..508: note = uncultivated organism
  REGION 1..508: note = MG91B-14 Effector
MADKECCVLTLPLQTEIWQEHIIEKRFAIMEHLKNQLIAKELRRLKNLERTKEYRNLIDEINNTPKEKRKALFSKRRKLLKDSGFSEYDFKDDIAGKNSLMQKHFIEHIATQIAHKLASDVWRSFDKFLFGNGRFIHFSKRGSLRSVASQKNGNGMTYKNGYFIWSGGQSKNKIILKIKVTPPKNEYEKEMLSKKIKYLRIVKKWVKTRYKYYLQFALEGKPVKKDRIVNKGNVGIDIGTQTVAISSADSVKLLELADKIDKNHTKKKILQRKMDRSRRYTNPQNYNEDGTIKKPPKGQRFSWYKSKKYIQLAGKVRELERKNADIRKYQHTCLANWILSLGDTVYVEQMNFSGLQRRAKETKIDKNGKYAKKKRYGKSLANKAPSMFLTILESKLNQYGGQLNKINTYEFKASQYDHTDDTFTKHNRSERWHILSNGDKNQRDLYSAFLIMNSDISLEHCNREKCNETYSNFKVLQDKEIERLYSDIRKGNCKNISSFGFQRNAKAM

SEQ ID NO: 165 - protein, 505 aa, unidentified
  REGION 1..505: note = uncultivated organism
  REGION 1..505: note = MG91B-15 Effector
MAKATTETCCLTLPLKLEKWQADRLDKRLEIARQIYNTLLRFELKKLRILINSEEYKDIENKRSETTDTNEKKKLNKKLDDLRKKAGFTEYSFKTDIKDFYKHFKDNIGSSVAVHGIAPQVWQAFDRLLYGNGREVHFKKRGSINSLRGYSVAGKSGGVEIMFRGDHIEWKGLHLPIKLDPNNVYECEMLQKRIKYCRIVKKQGRSKDHWYVQLMLEGKPAVKCDRHTGELLHPIGSGAVGIDIGPQTIAYVADKEVALKELADKVNNIEHKKQILQRKLDRSRRAANPDNYAPDGTIKRGVKLTHNKSKRYISTQRELADLMRKQAETRKRQHNELANHLLSLGDRFYVEDMQWPSLTHRAKKTEISEKTGRYKRKKRFGKSVGNKAPAMLISILDQKLRSRGKPGVTKVPTSVRASQYNHISDDYHKKELGERWNDMQDGRRIQRDLYSAFLLQHIDTDNNNFDKPTLFRDYENFVLLHDQYIEELKKLPKTLASMGIVRKAS

SEQ ID NO: 166 - protein, 521 aa, unidentified
  REGION 1..521: note = uncultivated organism
  REGION 1..521: note = MG91B-16 Effector
MAKEVFVIEFPLKVEKWQSDILNKRYEHLRQLYNYVQGKLLRQYIYFTQMGEYKACKSLKEKKDFFKEHIFHFKGIVGRNKKPLDISFTQYGISGLVSKLGQFSIGGKTYNDIGIGSTILANLAANVWASWDKRLYDIECERISFKKYGELNTFGSGKINKKYFCGFDLHLDRMKLDIKINGRTGKQAKFISLPIRYNLKHVDYEMNALAGGFDSIKVLTVVRRMVRGQYKYYLQMTIEGVKPQKGRHLGKGRVGIDIGPTTIAVSSNSTVSIDKLAAKCDNIEKDIQRINRKLDRSRRTMNPDNFNENGTIKRGVHLIWNNSENYKALRRQKAELQRKQAAVRKMQHIERANSLISEGDTFIIESNPIKGWTARAKETKISEKTGRIQKKKRWGKSVANHAPSMFITILENKVKSLGGTVIKADVKNAASQYDFTDGTFTKHELDERGVTLSNGDTHQRDMLSAFNLQHLIYDAKELKQYDQQRMKSHYPRFCELEQDEIERYRNKVKKDDRCTIAASRL

SEQ ID NO: 167 - protein, 566 aa, unidentified
  REGION 1..566: note = uncultivated organism
  REGION 1..566: note = MG91B-17 Effector
MGWKSKGRGGTLVDNEEKKEDGATCCLTLPLKLEKWQSDHLEKRFEIARQLYNTLVHAELKRIHRIERMPEYREIERQIEATTDKKEKWALKNQKKQFLRKNGINRNTFTTDMKRLYKHFNDNIGSNVAQHGIVSQVLTAFEKVYIENQSGEVHFKKPGDLRSVQGYSQKESGGKEIMFRNGKIIWNQQENDDRKKSPKYQQRKKRQNYKGVKGIAREFPVVFSKNNPYEAEMLTKHVKFVRILRKDGKTKPHWYAQLVLEGTPVAKYDPISGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 168 - protein, 512 aa, unidentified
  REGION 1..512: note = uncultivated organism
  REGION 1..512: note = MG91B-18 Effector
MAKCSTDTCCLTLPLRLEKWQEDRLAKRFEIARQIYNTMVRAELKKLRAIEQSEPYKANQEKIQALDWKTQADKQALKALYKERDKLLRNGGFSEYGFKYDIKNYYMHFNNNIGSNVAVHGIAPQVWAAFEKMLFKKEGKKVHFKKKGDIRSLRGYSVTRKSGGVEIIFRETYIEWKGLKLPLKLSPDNIYETQMLSYRVKYVRLLRKPGKRKDRWYAQLSLEGKPVIKYNPKTGEVRHPIGKGAVGLDIGPQTLAYSAATGADLVELANQVQNVEQEKRRLQRKLDRSRRATNPGNYADDGTIRHGIALTHNKSKRYLRTQQELKYLQHQQAEIRKRQHTELANHLLSLGDCFYVEKMVWTSLTHRAKETEISEKTGKIKRKKRFGKSVANKPPATLIGILQLKSAALGIPGVIEVPTSVKASQYNHQSGTYTPKPLSQRWNNMPDGERIQRDLYSAFLLQHYDPQTNTFDANSLHQDYPQFVQYHHDAVLRLYAMPKTPSSMGIKRRSLR

SEQ ID NO: 169 - protein, 548 aa, unidentified
  REGION 1..548: note = uncultivated organism
  REGION 1..548: note = MG91B-19 Effector
MEWKSTGRGGTLVDNEEKKEDGATCCLTLPLKLEKWQSDHLEKRFEIARQLYNTLVHAELKRIHRIERMPEYREIERQIEATTDKKEKWALKNQKKQFLRKNGINRNTFTTDMKRLYKHFNDNIGSNVAQHGIVSQVLTAFEKVYIENQSGEVHFKKPGDLRSVQGYSQKKSGGVEIMFREGKIIWNQQGNNDRKGVAREFPLKLSPDNSYEAEMLKKHVKFVRILRNEGKTKPHWYAQLVLEGKPAIKRDPASGAPKHPVGHGVVGIDIGTRTLAYSTDSKVDLIELADRVQNTEQEKMLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKKLKERWNIMPDGERIQRDLYSAFLLQHCRRDLPEYNRKKLQEAFGNELYRVFDREALQRDYPKFVEYHRETIQRLSALPKTIASMGIRRIIS

SEQ ID NO: 170 - protein, 511 aa, unidentified
  REGION 1..511: note = uncultivated organism
  REGION 1..511: note = MG91B-20 Effector
MGKSSTDTCCLTLPLKLEKWQEDRLAKRFEIARQIYNTMVRAELKRLRQVEQSPQYRSNKKAIQALDWKNTADKPKLKTLYRERDKLLKGGGFTEYGFKSDIKNFYKHFAENIGSSVAVHGIAPQVWAAFERVLFKKEGKKVHFKKTGDIRSLRGYSVTGKSGGVEIIFRGSYIEWKGLDLPLKLSPDNAYETEMLSYRVKYVRILRKPGKTRDRWYAQLSLEGKPAIKRNPASGNPVHPVGQGAVGLDIGPQTLAYAAEKEANLVELADRVQNIEREKHLLQRKMDRSRRAANPDNYAENGTIRRGVKLTHNKSKRYLRLQRELCYLQHCQAETRKRQHTELANHLLSLGSCFYVEDMAWPSLTHRAKKTEISDKTGRIKRKKRFGKSIANKAPAALINILRTKCVSLGIPGVVEVPTSLKASQYNHQSKEYTPKPLSQRWNDMPDGSRIQRDLYSAFLLQHYDPQSGNFDQASLEADYPQFVQLHNQSIQRLLTVPKTPSSMGIRRSVS

SEQ ID NO: 171 - protein, 466 aa, unidentified
  REGION 1..466: note = uncultivated organism
  REGION 1..466: note = MG91B-21 Effector
MAVMSVVTIPLKTRIYEEDIINKRMELCRKVYNNMLHDKLKDLRKMEHDKEYIASKSIILSAYKTDDKNEVKKIKNSDEYKKAQEVQKEKYKEYGFSEYAFASDVLKYIKLFNSNITATVATYTISKPMWAAFEKYLFADGRMIHYKSFNSISSVASDGKAGLKIVVNNKKSTRKPESPFGLSLKYGSNRAGKTINLPLIVDKNDFYMQEMICNNIKVVRLVRRKIKTHYKYYVQLTVVTAPSIKFDRCTGEIKNPINDGNVGIFIDTRKITLCHKNGLKQIDLIDKRQIKFEEEIGNINRFLDNSRRISNPENYNEDGTIKKGIVVNGQKRKLHWNNSNNYKKALAKKADIQRRLAEQRKIRAGEITNMILSLGNNIICNNYNFKAAQMRKKETEIDEKGKIKTKKKAGKAISYSAPAMLLTILNNKLVARGYNEIYKVEVDKDMTITDQTRILKAQELYMQLEN

SEQ ID NO: 172 - protein, 519 aa, unidentified
  REGION 1..519: note = uncultivated organism
  REGION 1..519: note = MG91B-22 Effector
MPTKQKEGGNRCVLTLPLLTEPWQEHIIEKRFKIIEHLKNQLISIELRKLKNLQRTKAYKNLIKQIESVPKSERTALYKERNKMLEGKGFSRNVFQDDLGVMQNQFAEHINVQISHRAAWDVWSSFEKFLFGSGQMIHYQKRGTLKSIASKSPKNGMHYFPESNTFKWSGGRQKKIGQKGEQKKVSIKPISLCLRVSPLKTDYEKEMVANNEIKYYRVVRKWMKTKYKYYLQITFAGKPADKHREISTGRVGIDIGTQTIAIASDKSVKLLELADKVNKNHIKKCELQRKLDRSRRYTNPDNFNPDGTVKRGTKGHKLKWNKSKHYLEIAGKIRELERKNADIRKYQHTCLANDILAMGNQIYIEPMNYKGLQKRAKETQVDEKGRYKKKKRFGKSLANKAPAMFVTILKTKAEQHGGEVYEVDIMLYRASQCDHTTGEYTKHGLNDRWRILGNGDKVQRDLYSAFLIKNSNKTHDEIDLDKCSKTYDSFKKLHDKEIENIRNEVILRKRRNISSYGIA

SEQ ID NO: 173 - protein, 536 aa, unidentified
  REGION 1..536: note = uncultivated organism
  REGION 1..536: note = MG91B-23 Effector
MDADEYKKLLKKFHKLSDRHILVAEADMSYPDVQKVVNLSDRIRKAGNELVSLMKKNYEQLMRTKKYRKLLQLYGSTEDKKKRKTYAGQLNEMQEQYNVTWDFCRASMIPIGKKYGIDAIFALTKAEDVWRGVEKCLYGNGRMLHFSKYGDFPCIRAKQINRGIPMSVKNDELKFKLGKLVFGIQVKDRFQTDEVNAVLDYLAQPEVIDNKAVKTLQDEACCISTYRPCYATLVPKFIRGKYRVYLHLVIEGKAKPKYDSSGNPRHKYGRGVVGADIGTQTVAYTSDTETGLKNLSERGNSIQKSERLERLYYRAMDRSRRATNPKNYNADGTIKKGKKKWTYSRHYKKLKAKHAELCCINATNRQLAINEDVNHLRCLGDTFVTEPKNAARLMKRAKETTVNNKGKINRKKRFGKSVKNRCPGGFQAAVENKFKTSGGTYIEVPNNYRASQYDHTADDYIKKKLSDRMYKLADGTLVQRDWYSSFLLYCYNYRTNDIDKKKCAAEFKKCYSMEKDLIEWIKENKIKVLNSGIKIA

SEQ ID NO: 174 - protein, 849 aa, unidentified
  REGION 1..849: note = uncultivated organism
  REGION 1..849: note = MG91B-24 Effector
MKEEDIIKFSDSLKGLGTNKKGVALILRNPIKITEDSAVQGVIIDNKPDRKEGIDRLDYIVYDRENGMHRSPATEGNFDQEVPYHILSRFEDLMDCGKLIPVVFEDSVNPFSTDITFKHNDPLKNDKKAFWNYPLDKEQFEASSSVRFYKSVLAPRNFSESNTFNCKTEEASLYFAVENARRLSKRGSAVRDFRNKDIWTVSLDNADESKPLDVETAEFFATKSIRSSILNMGDTKVFQALFDSKKNAIAFASDYANWMTKAEIAVGKLLGFNIPSNIQDQGMDLLGGDKKYIDCYLQKNFYTEEFQERPFGELLDNTLKWIREYGANPDEENIKAVSESLDKIAFDKYVVEGIHLDYFFQEPITLDDGRKIVGFGKNLGKDNVTWAAKAYTTNADNTAVEKVDFKDLPHDGLKKAIEKLDSPYHPHIDFGDKKLNVYKMDRVAYSLYQRISNVGAKSFTPSEASQAELFFKQLPEEIRQPMAVYLENLVRKNINEKEVSLPEKWFTSAFEELEKIAKDGLPIKYNLKHADYEMTALAGGIDSIKLLTVVRRLVRGKYKYYLQITIKGKKPQKGRELGSGKVGIDLGASTIAVCGDNIVSIDKLAAKCDYIQKDVKKIQRKMDRSRRANNPDNYNENGAIKKGKKLVWKNSNRYKALCKQYAEIKRKQAAVRKIEHIERANNLLKEGNIFITEKNPIKEWAKKSEETEINPKTGKTKSKKKYGKSISVHAPGMFIEILKNKVVSLGGKFIKDNPENAASEYDFTNDTFTDHGFDRTITLSNGDKHQRDTLAAFNIKNRKYDEEEPKQYNREQMQKEYARFCELEREEIEKYKNGTKKDYRRTIAAYKLY

SEQ ID NO: 175 - protein, 486 aa, unidentified
  REGION 1..486: note = uncultivated organism
  REGION 1..486: note = MG91B-25 Effector
MSNFVVEFPLITEPYQEHILNKRFEIGRIIYNTLVNQTKKIYKEMIKTKKYRELQNKLSDDEKSNKLVWKEINNIRKENGFSEYSFHKLVSKSQKYFKKNIDSFTAQKIATRLWLTYDKFFYGNGEKIYFKKFGTLNSLEGKSNSSGIRFKNNKIYWNKLIIPVKINYSNSYEYEALQHKICYNRIIRKYVKNKYKYYVQIVFEGIPPIKINQNTGEVKHHIGNGDVGLDIGTSTIAISSMTDVKIFELADKVKNIEREKLLLQRKMSRSRFSTNPNNYNSDGTIKSQGNRKVKWIKSNHYIKYQKQLRELYRKQTAVRKYQHECLANYIISLGNKFFVEKMNFKALQKCTTKTEKNEKGKFKRKKRFGKSLGNRAPSMLLTIINRKLSYYGTELIEINTYKAKASQFNHIDNTFKKKKLSQRWNDLNGIKVQRDMYSAYLIMNINKDLETFNMKKCNDRFDNFLKLHNKEVNRLKNHKNLSCIAI

SEQ ID NO: 176 - protein, 495 aa, unidentified
  REGION 1..495: note = uncultivated organism
  REGION 1..495: note = MG91B-26 Effector
MSDSSTEKSVFVVTFPLKTEKWQEDRINKMMRLLTVFYNEKQKVLLERWMHIRNSAEYKEHKEEKSKNSLTAYMRNFGFSEFGFKEFFKDKNQTDSPYITHGLNSAILRNLAKSAWSAWSKKLEEKKKNIFIHTDKDVSIIKSDFKSSNNKISGFDVNYEDYSITMSASKPHVHTMFTIPFVVDRNSEYELFALGEIQNDPTKLRNLAIVRKEVRGKYKYYVQFSIAGKPYNKGRQLGSGVVGIDPGPSKIAVVSDTAVRIIPLAKSIERDERETRRLQRKLDRSRRAMNPDNYNEDGTLSKGKHEWVKSHHYEETRSILADRQRKLAAKRKIAHNELANELLQMGNEFHVENNSFRSMQVRAKETTKNANGKNRTKKRFGKSLANRAPSEFLIILENKVKQYENGVYVDIPDSIACTQYDFTSGDFSAHELRERTITTSDGIRHDRDALAAFNMKFVRTEQVVEKKKVKKAVDNFDNESMAEFYPLFCQMENKE

SEQ ID NO: 177 - protein, 510 aa, unidentified
  REGION 1..510: note = uncultivated organism
  REGION 1..510: note = MG91B-27 Effector
MFVVELPLKVEKWQSDVLNKRYELIAKIYNYAQKKLLRQYIYLTQMDEYKKCKTKKEKSNFFKNHPFYFKDITSKITFSEYDVTAFVEKIIKKNVSSDKTYKDFGINTSITECLGKHIWSAWEKKIYDKGKKISFKNKGELNTISIRKKSEYFGGLDVDIEHMILSFNINGKIKAKAKKIILPINVKNLTEYEMYALQGGYDSINVVTIIRKTIRGKYKYYVQFTIEGEKPQKGRTLGKGNVGIDIGPSTVAISSLNGVTIDTLAEKCDNIESKISDIGRKMDRSRRATNPQNFTEDGQIKRIDRKKGEKRVWKCSKRYVKLRNKKHELLRKQAVIRKLQHNIKANEILSLGDTFIVEDNPTSAWAKRAKGVRKNKNGKIQTNKRFGKSIANHAPSMFVTILQNKVDSLKGKFVKVDCKNAASRFDFTNGDFTGHNLDERKITLSNGNTHQRDMLAAFNLQHIDLTNDDLKKYNVDEMVEDYPIFCKLEKEEIGKFINGQKKSRSCTVGY

SEQ ID NO: 178 - protein, 528 aa, unidentified
  REGION 1..528: note = uncultivated organism
  REGION 1..528: note = MG91B-28 Effector
MNEVFVLELPLQVEKYQADILNKRYEQLRQLYNYVQGKLLRQYRYFEQMKEYQACKTFKEKRDFFRSHPFHIDGILGSNKELFDITFDEYGINGFVGKLGKKQVSAGVTYADLGITSTLLDRLSLAVWAAWEKMLYDYHARRVSFKKYGELNIFGSRKKTNGSGKKMFSGMELHLEKMEVAIKLNGKVGKAAKFITLPILHNPKHVDYEMWALKGGFDSVTVLTIVRRLVRSQYKYYVQLTIEGEKPQKGRTLGKGNVGIDLGPTTVAVCGENVVSIDKLASKCDNIQEKITRLSRKIDRSRRANNPQNFNEDGTIKRGIKLVWKKDKDLKRYSKLKRELAELRRKQAAIRKQQHINRANALLKEGDTFIVENNQISSWTTRAKETKVNEKTGRIQKKKRFGKSVANHAPSMFINILENKVKSLGGKVVKVDTKNAASQYDFTDGSFTKHELKERNVTLSNGDMHQRDILAAFNLQHLKYDEAEAKQYDTEAMSADYERFCQLEQEEIQRYKNKEKKDDRGTIGAEKL

SEQ ID NO: 179 - protein, 525 aa, unidentified
  REGION 1..525: note = uncultivated organism
  REGION 1..525: note = MG91B-29 Effector
MNEVFVLELPLQVEKFQVDILNKRYEQLRQLYNYVQGKLLRQYRYFEQMKEYQACKTFKEKKDFFRSHPFQINETLKGNETAFKVSFDEYSINSYIEKLVQKSVGSNMTYSDLGFTTNILDHLSASIWAAWNKKLYDFKAKRISFKKYGELDSFSSRRKVVRGVVYFSGIELHLDTMELAIKMNGKVGKEAKFITLKMLHNPKHAEYEMWALKGGVDSVKVVKVVRRLVRSQYKYYVQLTIEGEKPQKGRTLGRGNVGIDLGPTTVAVSGENVVSIDKLASKCDNIQEEITRLSRKIDRSRRANNPQNFNEDGTIKRGIKLVWNDSNRYKELRRELAELRRKQAAIRKQQHIDRANSLLKEGDTFIVENNQISSWTTRAKETKVNEKAGKIQKKKRFGKSVANHAPSMFVSILENKVKSLGGEVVKVDTKNAASQYDFTDGSFTKHELKERNVTLSNGDTHQRDMLAAFNLQHLKYEEKDKTKQYDTDAMKKDYERFCQLEQEEIQRYKNKEKKDDSSTIGAGEL

SEQ ID NO: 180 - protein, 464 aa, unidentified
  REGION 1..464: note = uncultivated organism
  REGION 1..464: note = MG91B-30 Effector
MAENKIMSVVTLPLKTEKWQEDILQKRFELCRKIYNNMLHYEMKQLNKVLHDKRYIDSKKIITETYKIADDKEKRARKRTEEYKQAVKISNEVLKEYGLTDYGFKNEVLKQRAVYKTNISSTVAQLSVASPLWAAMDKYLFGNGKTLHYKKYNTLNSMASDGKSGIRMVDKDGKTIFSRESAMPIWINMSVPKGKTLLMPVIIDKKDLYKIEMMSRKLKVIRITRKLCKAKYKYYVQFTVEGRPAIRYDKDGNIKNPVGNGKVGIYIDTTSITLALENGDIKTISLQHDNTSTEKIADLQKYMDNSRRATNPDNYNADGTVKKGITIDGKKSKLRWNYSNGYNRARLELKNLYRVDAENKKLERQILANKILSYGDNFAINDYPFQYAAMRKKMFNENTLKDEKHKKAGDKIGNNAPATLVVLLDSKLKAKVGAGVTKVQLKDIDYSMENYRERYAQKILEYLQ

SEQ ID NO: 181 - protein, 456 aa, unidentified
  REGION 1..456: note = uncultivated organism
  REGION 1..456: note = MG91B-31 Effector
MSNAENTSGRYILTLPLKTELYQEHILEKRFRLGEQLYNKFLNFEKKKYFEMTKRKDYRNAQSLIKNFTYQIKSLNNASDISKDTKKMIQDFKKQRKEQYDIIYNIKKMFNWSEFGFGKDMKKYRGYYKKNLNSQICNNLAVRCFRAFEKMEEEFIKDQYRSDEDKVNPCVHYKRKDSLKSLCGSQNTTGIRFILGDERNVGTVKWQGLEFSIDLVKCTLYEWKTLQSEICYCAVKREKIKGKWKYYIQLTLKGHVPDKFNKQTGELKRQLGKGNVGLYFTSTSLTVSTEDGTKTYPLAIKNHEDKKTELLQKMDASRRATNPDNYNDDGTSKEKNEIKGWHFSKAYKKYRAELYEIFRKECEEKKLLQEVLANEVIASGDVFNCNKMDFKFLQRNLGKKILTASPSMLKSVIERKLSYHDVSINEISYSKLNPVLEEKNISKKNLEDMATLISQF

SEQ ID NO: 182 - protein, 485 aa, unidentified
  REGION 1..485: note = uncultivated organism
  REGION 1..485: note = MG91B-32 Effector
MKRFEIARQIYNSLLHAEQKKLRRLEQTPEYREIQRKLQELYREKAEKSRAFREIAKKRTALLKRAGFTEYAFKTDMKAYYKHFKENIGSSVAVHGIASQVWTAFDRKLYGNGKHVHFKRPGEIRSLRGYSAAGKSGGVEILFRGTHIEWKGLCLPLKQKPENAYETEMLSRRVKYVRILRTPGKHRDRWYAQLVLEGRPAVKRNTETGELLHPIGKGPVGIDIGPQTVAYVSQGEASLLELADRVRNIEQEKRKLQRKMDRSRRATNPENYAEDGTIRRGIRLTRNKSGRYRKLQKELAFLQHHQADIRKQQHIALANHLLSLGDRFYVEDMAWPSLTHRAKETVISPKTGRYQRKKRFGKSVANKAPAMFLGILTQKCVSLGLPGVVKVSTSVRASQYNHLTETYQKKPLSQRWNVMPDGRKIQRDLYSAFLLQHMTPQLDGFDPDGLRQDYESFAVLHEEAIHKLRLVPRTLTSMGLQRKCS

SEQ ID NO: 183 - protein, 537 aa, unidentified
  REGION 1..537: note = uncultivated organism
  REGION 1..537: note = MG91B-33 Effector
MIIDGGYKKLLKQFHKHSDRHILVIETDMPYSDILKVVALSDKIRKAGNELVGLMKKNYAQLLRTKRYRKLLFLYGNTEDKDKRKIYANQLNEMQKIYNVTWNYCRTSMIPIGKKYGIDAVFALTKAEDVWRGMEKCLYGNGNTVHFSKHGDLPCIRAKQISRGIPISVRNDKLQFKLGKTSFGIQVNDRFQQDEVNAVLSYLVEPEIMDNKAVNTLIEDAYCIDTYRPCYATLVPKMIRGKYRVYLHLTIEGKTKLKYDKHGNPRYKYGKGIIGADIGTQTVAYTSDTEVGLKNLSERGNSIQTSERKERLLYRAMDRSRRATNPQNFNEDGTVKKGRKTWKYSNHYKKLKTKHSELCRINAVNRQLAINEDANHLRGLGDTLVTEPKNASKLMKRAKETTVNSKGKFNKKKRFGKSIKNRCPSGFQTTVEKKFKTTGGTYIEVPNNYRASQYDHTVDDYIKKKLSDRLYKLQDGTEVQRDWYSSFLLYCYDYKIQNIDKNKCITEFDNCYKKEKALITWIKTHKIKVLNSGIKTA

SEQ ID NO: 184 - protein, 499 aa, unidentified
  REGION 1..499: note = uncultivated organism
  REGION 1..499: note = MG91B-34 Effector
MADNDARCVLTLPLITEPWQEHIIETRFRIMEHLKNQLIAMELRKLRNLRRTREYRALEEDIANTPKGSRGELRRKRRKILRDAGFSEYDFKDDMAGTNSKMQKHFAEHIAAQIAHKAASDVWRAFDKCLFGSGKGIRFHAHNTLSSVACQEIGNGMSYVNGKFVWKGGRCENSICLSVPVKPPDTVYECEMLKKQVKSLRIVRKWMKSKYKYYLQFTLTGEPIKKTREVGFGNVGIDIGVQTVAIVSSSSVFLLELADKINANHSRKKQLQQKMDRSKRYSNPSNYDKNGVIIRSPSGQRLRWEFSKKYRKYAGIVRNLERKNADIRKYQHTCLANAVLPLGNRVFVETMNFSAHQNRAKETKKDSNGRFLKKKRFGKSLANKAPAMFISILKYKLALYGGNLFEVDRYTFKASQYDHTTGECTKHSLSERWRTLGNGDIVQRDLYSAFLLMNSNTSLSSCDATACAHTYTDFKTLSDYEIHRIKSEGIRLISSFGIA

SEQ ID NO: 185 - protein, 512 aa, unidentified
  REGION 1..512: note = uncultivated organism
  REGION 1..512: note = MG91B-35 Effector
MEKTKGSDDTCCLTLPLKLEKWQEDRLARRLELARQIYNTLMHAELKKLRNVQQSPEYRRMQAEMRALDWKKEADKPPLRKLYKERDKLLKKAGFTKFGFKNDMKYYYKHFNQNIGSSVAVHGIASQVWTAFDKMLFQKDGKKVHFKKYGDLHSVQGYSAAKSGGVEIMFRGTCIEWKGLELPLKRKPDNAYETEMLQKHIKFVRILRKPGKNRDRWYAQLTLAGKPAVKRDAVSGRQIHPLGHGPVGLDIGPQTLAYAAETEADLVELADLVQDIELEKRRIQRKMDRSRRAANPDNYAEDGTIKRGVKLTHNKSRRYLRLQKKLSHLQHCQAETRKRQHNELANHLLTLGDCFYIEKMNWTSLTHRAKKTEISEKTGKIKRKKRFGKSIANKAPGMLIEILKRKCLSRGLPKVAELPTTLKASQYNHLTKEYTKKSLSQRWNTMPDGKRIQRDLYSAFLLQHYNPQLQNFDQEALEKEYPQFVLLHDQAIERLSAIPKMPSSMGIRRSVS

SEQ ID NO: 186 - protein, 490 aa, unidentified
  REGION 1..490: note = uncultivated organism
  REGION 1..490: note = MG91B-36 Effector
MSSQGERCVLTLPLLTEPFQDVIIEKRFKIMEHLQNSLIAYELRKLKSLERTRAYRELCERIRNASDKERKTLSRQRSKMLKEAGFSEYQFQDDITPMQKHFVEHIFVHEAHRAAQDVWRAFEKYLFGSGKMVHFKRRGTLSSIANKTSGMYLRDGWFEWNGGQSKQSIRLKIRVMPPRNAYETEMLKRPVRNFRVVRKWMKCRYKYYLQITLEGNPVVKDRPVAEGRVGIDIGTQSVAIASLNGVCLLELADQVNKNHARIKYLQQKMDRSRRATNAGNFAPDGTIRRGIRLTWSESNHYKRLAGTVRRIQRKNADIRKYQHYCLVRDVLMLGNQVYVEKMNFQGLQRRARETTVNERGRFKRKKRFGKSLANKAPAMFISLLEQKLHSSGGNLYRVNTWEFKASQYDHLSRKYNKKKLSQRTHRLGNGDVLQRDLYSAFLLMNADSSLRFPDQKKCDETYPIFKSLHDKEVKRISNDSKYHLTSFGIA

SEQ ID NO: 187 - protein, 512 aa, unidentified
  REGION 1..512: note = uncultivated organism
  REGION 1..512: note = MG91B-37 Effector
MGKSSTDTCCLSLPLKLEKWQEDRLAKRFEIARQIYNTLVHAELKRLSQVKRSQQYREIENKIRALDRNDQEDKKELSLLHKLRDDLLRKSGFNDYGFKADIKYFYKHFGENIGANVAVHGIAPQVWSAFDSVLFKKRGKKVHYKKPGDICSLRGYSVTGKSGGSEIMFRGTYIEWKGLKMPLKLSPDNIYESEMLDHRVKYVRIVRKPGKRKDRWYAQLSLECKPVIKCDPVSGEMRHPVGKGPVGLDIGPQTLAYCALSEAGLVELADQVQDIEREKQRLQRRLDRSRRAANPNNYAADGTIKRGVKLTHNKSKRYCILQRELRYLQQQQAETRKRQHTELANYLLSLGDRFYVEDMNWSSLARRAKKTEISEKTGRYKRKKRFGKSIANKAPGSLIDILKQKCESLGIPGVEKLPTSLLKASQYNHQTGEYTPKPRSQRWNDMPDGERIQRDLYSAFLLQHYDPVSKGYDQAALQRDYPQFLQHHHHSIQRLTAKPKTPSSMGIHQRLR

SEQ ID NO: 188 - protein, 536 aa, unidentified
  REGION 1..536: note = uncultivated organism
  REGION 1..536: note = MG91B-38 Effector
MVNDDYNKVLKQFHKLSNRHILVAETDMSCSDIQKVVKLSDRIRKAGNELIGLMKKNYDQLMRTKRYRGLLKLYGSTKNKQKRKMFADQLNEMQKQYNVTWDYCRKSMIHIGKKYSIDAVFALTKAEDVWHGVEKCLYGNGKTLHFSKHGDLPCIRAKQINRGIPISAKNNQLQFKLGKSVFGIQIKDRFQTDEVNAVLDYLAAPEIADCKAVQTLLEEACCISTYRPCYATLVPKLIRGKYRVYLHLAIEGRAKPKYDRFGNPRHKYGKGIIGADIGTQTVAYTSDTETGLKNLSDRGNSIQTSERLERLYYRAMDRSRRATNPQNYNDDGTVKQGKKTWNYSNHYKKLKAKHAELCRINVANRRLAINEDANHLRSIGDVFITEPKNAAKLMKRASKTTVNNKGKINKKKRFGKSIKNRCPGGFQTAVENKFKASGGTYIEVSNNYRASQYDHTADDYIKKKLSDRMYKLADGTLVQRDWYSSFLLYCYDFRTSDIDKDKCAAKFDKCYSMERELIEWIKTNKIKVLNSGIKIA

SEQ ID NO: 189 - protein, 536 aa, unidentified
  REGION 1..536: note = uncultivated organism
  REGION 1..536: note = MG91B-39 Effector
MDNEEKEKDGATCCLTLPLKLEKWQSDHLEKRFEIARQLYNTLVHAELKRIHRIERMPEYREIERLIEATTDKKEKWALKNQKKQFLRKNGINRNTFTTDMKRLYKHFNDNIGSNVAQHGIVSQVLTAFEKVYIENQSGEVHFKKPGDLRSVQGYSQKKSGGVEIMFREGKIIWNQQGNNDRKGVAREFPLKLSPDNSYEAEMLKKHVKFVRILRKDGKTKPHWYAQLVLEGTPVAKYDPISGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 190 - protein, 615 aa, unidentified
  REGION 1..615: note = uncultivated organism
  REGION 1..615: note = MG91B-40 Effector
MAKEVFVLTLPLKVEKWQADILDKRYELLRQTYNMVQQRLLRQYIYFSQQKAYKACETFVQRRDFFASHPFHFKGIKGKNGESSDIKFPYTYESRSSNTGKRPSGGISDYVSKLWSHRVGQDKTLADFGINSYLLEELGLHIMKAWEKRLYDPQSSHVSFKKYGDVNSFGSRYKKDKKSNRITFPGFAIDLVKGYIVYNTEGKRVKTNPFVLPIDFSREKKEYALESLKGGVDSIRKLLIVRRCIRGRNKYYLQLSIKGEKPQKGRTLGKGNVGIDIGPSTIAVSSLQGVHFDKLANKCDNIEHELHLLNRKIDRSRRANNPQNYNEDGTVKRGVRLVWKKDKDAKRYANLKQIRKELHRRQAAIRKQQHLEMANALLSQGDTFIVENNPVDAWVRRAKETTKTKTGKNRCKKRFGKSVANHAPATFVSILKNKVLSLGGQFHEVDIHNAATQFDFTSQQFTKHELGERSVKLSNGNTHQRDMLSAFNLQHLRAQSEENKDYDIEQMKADYPIFCKLETAEKERIKGERSVERDVLFYIGASTQDMRQQPQKSTVVNRHNGGVGRKHCNTRLGSRKKENAEITGSGLVRESIERVSQVPDEYHHFGIQSVILDGL

SEQ ID NO: 191 - protein, 528 aa, unidentified
  REGION 1..528: note = uncultivated organism
  REGION 1..528: note = MG91B-41 Effector
MEEVFVLELPLQVEKYQADILNKRYEQLRQLYNYVQGKLLRQYRYFEQMKEFQVCKTFKEKRDFFRSHPFHINGIYGNDKELLDITFDEYGINGFVGKMGKKLVNANTTYADLGITSTLLDRLSLAVWAAWDKMLYDYRARRVSFKKHGELNIFGSRKKTNGSGKKMFSGMELHLEKMEVSIKLNGKVGKKAKFITLPILHNPKHADYEMWALKGGFDSVKVITVVRRLVRGQYKYYLQLSIEGEKPQKGRTLGKGNVGIDIGPTTVAVSGENIASIDKLASKCDNIQKEITRLARKIDRSRRANNPQNFNEDGTIKRGVRLVWKKDKDCKRYAALKREMAELQRRQAAIRKQQHIDRANALLKEGDAFIVENNQISGWVRKAKETKVNEKTGKIQKKKRFGKSVANHAPAMFVTILENKVKSLGGEVVKVDTQNAASQYDFTNNSFEKHELKERSVTLSNGDTHQRDMLAAFNLQHLKYDDAEKKQYDTEAMAQHYDRFCELEQAEIQRYKNKEKKDDRSTIGAGDL

SEQ ID NO: 192 - protein, 526 aa, unidentified
  REGION 1..526: note = uncultivated organism
  REGION 1..526: note = MG91B-42 Effector
MKEVFVLEMPLLVEKYQEDILNKRFEQLRQLYNYVQGKLLRQYKYFEQMAEFQICKTSKAKREFIKTHPFHINGVIGRNKDLLDISFDEYGILGLVTKLGQKPLGSGKSYADMGISSTILANLSNNIWSAWNKKLYDYHAKRISFKRYGELNSFGSGKIVQKKDNDVKEYFPGFILHLERMELDIKTSKSGKNETCITLPILHNPKHADYEMWALKDGSTSIKVLTIVRRLVRGRYKYYLQMSIEGEKPQKGRTLGKGNVGIDIGPTTVAVSGENIASIDKLASKCDNIQEEITRLARKIDRSRRANNPQNFNEDGTIKRGVRLVWNDSKRYKALRKEMAELQRKQAAIRKQQHIDRANALLKEGDTFVVENNPIKGWQARAKETKIDEKTGRFKSKKGRGKSIGNHAPAMFVTILENKVKSLGGEVVKVDTHNAASQYDFTNESFTDHGYDRKITLSNGDTHQRDMLAAFNLQHLKYDETEKKQYDTEAMVQHYDRFCELEQEEILRYKNKEKKDDRSTIGAGDL

SEQ ID NO: 193 - protein, 517 aa, unidentified
  REGION 1..517: note = uncultivated organism
  REGION 1..517: note = MG91B-43 Effector
MKKKSTEETFIMELPLKVEKFQADELNKRFELMRRIYNSAQNKLERQHEYLLSFKEYKECNTKKAKGEFINNHPFSFKGVLNKNGEPMNITFTEFGLKSYLADFAKKKVDSQYTYSDFGINTSMLMSMGMAAWSAWKKVIYDPKAKDIHYKKYGDLKSIAVDWEPKHQAGMYLDVCKGVLKLNINGRRGQYAKYLTFPIKFGRKERQLWYEGDALKGGEDSFKGLAIVRRKYNGQFRYFLQLTIRNCTPTKDRTLGKGRVAIDLGIYKIAVYTGDQVYFFPLAPNCREIDEKIAVLQRQMERSKRISNPQFYNADGTYIKAKKHEHRKWNFTKEYLKLRAMKAELQRKQAAIRKQDHIDLANHLLSFGNEFVIEDNDVKAWAERKKEEKKREKDGKNLSKKGLGKPIGSHAPAAFVTLLANKVKALRGTFTKVAVDNGATDYDFINDTFTPHELKERYITLCNGVTHQRDSLAAFNLYYIDTNPEKSRCYDRKAMKRGYKKFQELEKEALKQWKKPV

SEQ ID NO: 194 - protein, 511 aa, unidentified
  REGION 1..511: note = uncultivated organism
  REGION 1..511: note = MG91B-44 Effector
MAARSKTDTCCLTLPLRLEKWQADRLAKRFHIAEQIYNAMLLSWIKKLRVVEQMPEYRAIQSQLQKLYEEDKQKTDAAKELFSRRKEILKKNGFPEYSFKTGIRKYYAHFKENIASNVATHCIASQVWDAFSKKLFGNGGRLHFKRAGDIHSLRGYSKSGESGGVEIIFRGTYIEWNGLRLPLKLDPQNPYEAEMLAKRVKYVRILRTPGKSIDHWYAQLALEGKPTVKRNAATGEPVHPSGRGAVGIDIGPRTIAYVSENEVGLRELADQVENIERDKRRLQRKLDRSRRATNPDNYAPDGTIKRGIKHTHNKSSRYRRMQKELSLLQHRQALIRKRQHTELANHLLSLGDCFYVEDMPWKGLARRTKETTVSPTTGRYRRKKRFGKSISNKAPGTLILILEQKCKSRGVSGVIRVPTNIKASQYNHMTDTYKPKPLSQRWNDMPDGKRIQRDLYSAFLLQHVTPELDGFERTKLIEDYDGFIHLHDKEIHELTLAPKTIASMGISRTRS

SEQ ID NO: 195 - protein, 505 aa, unidentified
  REGION 1..505: note = uncultivated organism
  REGION 1..505: note = MG91B-45 Effector
MARAQTDTCCLTLPLKLEKWQSDRLDKRLEIARQIYNTLLRFELKKLLRLEESKEYTDIQNSLKEITDNTERNKLYKQIDKLRQKAGFSEYAFKTDIKEFYKHFSDNIGSSVAVHGIAAQVWTAFDRLFYGNGKAVYYKRRGELNSVRGYSVAGKSGGIEIIFRGNYIEWKGLKLPIKLDLTNTYENEMLQRRVKYCRLLKKPGRTKERWYVQLMLEGKPAIKVDKNTGELRHPVGRGAVGIDIGPQTIAYVSDSEIDLKELADQVNNIEREKRVLQRKLDRSRRATNPDNYAPDGTIKRGVKLSHNKSKRYIKTQRELAALQRQQADIRKRQHNELANHLLSLGDCFYIEKMEWPALTHRAKQTEISEKTGKDKRKKRFGKSVGNKAPATLTGLLDQKLKSRGLSGIIEVDTKVRASQYNHLTDDYHKKELSERWNFMPDGRRIQRDLYSAFLLQHMNEQQDGFDKTALQRDYERFVIQHDQCIEELKRAPKTLASMGIVRRAS

SEQ ID NO: 196 - protein, 528 aa, unidentified
  REGION 1..528: note = uncultivated organism
  REGION 1..528: note = MG91B-46 Effector
MEEVFVLELPLQVEKYQADILNKRYEQLRQLYNYVQGKLLRQYRYFEQMTEFQVCKTFKEKRDFFRSHPFHINGILGSNKELLDITFDDYGINGFVTKLGLKSIGNGKTYTDIGINSAILEKLSDNIWASWDKMLYDYKAKRISFKRYGELNSFGCRKKLNGSRKKVFSGMELRLDKMELAIKLNSKTGKNAKFITLPILHNPKHADYEMWALKGGYDSVKVITVVRRMVRGQYKYYLQMSIEGEKPQKGRTLGKGTVGIDIGPTTIAVSGENIVSIDKLAAKCDNIEAEIQRLARKIDRSRRANNPDNFNEDGTVKRGVRLVWKKDKDCKRYAALKREMAELHRRQAAIRKQQHIDRANELLKEGDTFIVENNQISSWVRRAKETKVSEKTGRIQKKKRFGKSVANHAPSMFVTILENKVKSLGGEVVKVDTQNAASQYDFTNNSFEKHELNERSVTLSDGKKHQRDMLAAFNLQHLKYGESEKKRYDTEAMNQHYERFCELEHEEIMRYKNKEKKDDRSTIGAGDL

SEQ ID NO: 197 - protein, 603 aa, unidentified
  REGION 1..603: note = uncultivated organism
  REGION 1..603: note = MG91B-47 Effector
MAKENFVLTLPLRVEKRHADILDKRYELLRQMYNMVQQKLVRQYIYFSQQKAYKACENFEQKREFFNTHPFHFKGIKGKNGELCDIKFPYTYESRSSSSGKRPSGGISDYVSKLWSHKVGADKKLVDFGLNGSIMEELGLHVMKAWEDRLYKFKARRVSFKDYGEINSFGCRYHKGNFAGFTINLEEGFITFNANGRRGQFAEIITLPIDFSREKKGYAEEALKGGVDSLRKLIIVRRCIRGRNKYYLQLTISGEKPKKGRTLGMGNVGIDVGPSTIAISSLQGVHFDKLADKCDNIEHKLYLINRKLDRSRRANNPDNYNPDGTIKRGVRLVWKKDKDVKRYASLKQQRKELFRKQAEIRKQQHLEMANAMLSQGDTFIVENNPIDAWVRRAKETKKTKTGKNRCKKRYGKSVANHAPAMFVSILKNKVLSLGGEFREVNPGYAATQFDFTNGVFTKHEVSERSITLSNGRTHQRDMLSAFNLQHICNELKEYNITQMQADYPIFCRLETAEQERFKEARRNERDALYFVGAFAKELRHSPQEETVANRHNGGVGRKHSNTLCTENAKITGSGLVRESIERVSEAPEECYHFDCQPIILDGL

SEQ ID NO: 198 - protein, 614 aa, unidentified
  REGION 1..614: note = uncultivated organism
  REGION 1..614: note = MG91B-48 Effector
MAKENFVLTLPLKVEKWQADILDKRYELLRQVYNMVQQKLLRQYIYFSQQKAYKECKTFQQKREFFDSHPFYIKIDDKPCEIKFPYTKKNSNTAPNTISNYVSGLGSHRVGQDKTLSNLGINSIILEELGIHIMKAWGDRIYNSKSEHISFKKYGEVNSLVSRYKKKKQTGKVYFPGFTINLEERYITYNTNGRQGNQAKLIKLPIDFSREKKRYAAEALKDGIDSIRKLSVVRRCIRGKNKYYLQLVIDGEKPQKGRTLGRGNVGIDIGPSTIATSSLLGVHFDKLADKCDNIEHELYLIDRKLDRSRRANNPQNYNEDGTYKRGVRLVWKKDSEVKRYAKLKAARKELYRRQAEIRRRQHLEMANAMLQLGDTFIVENNPIDAWARRAKETKKTKSGRTRCKKRYGKSIAHHAPAMFVTMLENKVKSLGGEFYRVDAYGYAATQFDFTNKKFTKHAVNERSITLSNGHTHQRDMLSAFNLQHARLEGEKTKSRDKEKKVESYDIEQMQADYPIFCRLEAEEQKRFRVAWHNEKDALYFVGASGKQLRHQSQEKSAANRHNGGVDRNHCNTRLGKENAEITGSGFARESIARVSQVPDEYHQHGQQSIIIDGL

SEQ ID NO: 199 - protein, 446 aa, unidentified
  REGION 1..446: note = uncultivated organism
  REGION 1..446: note = MG91B-49 Effector
MQAVLYQEGETAVTETKESKNRCVLTLPLLTEPYQEHIIEKRFKILEHLQNSLIAMELRKLKNIQRTRAYRELEQSILQLPKEKRRSLYQQRRQMLEKAGFSRNAFKDDITPMQKHFVGQIATHVAHHVASDVWRAFDKHLYHGAKKVHFKHRGTLSSAANQTAGNAMSMKDDMFIWSGGQSKDKITLRIRVQKPDTDYERDMLQKKVKYFRVVRKWVKTRYKYYLQLTLVGEPAARHRPIGEGRVGIDIGTQSVAIVSAEQAHLWELADRVNANHEKMLHLQRKMDSSRRSMNPENYNEDGTIRRGIRLRWIQSNRYRKLAGQIRELQRKNADIRKYQHVCLANYILTLGSEIYVEDMDYRAMQRRAKKDRKEQTWPIQAKKTFWEVLGQQGTVHVSHHFGHQAAAGNRERASPREQVVFPRQSIRSSVSKVHPQNTRNKKLYAQ

SEQ ID NO: 200 - protein, 476 aa, unidentified
  REGION 1..476: note = uncultivated organism
  REGION 1..476: note = MG91B-50 Effector
MGVCSVLEMRLKTEPWQESLIDKKMECARAVYNATLSETMKNYNRIINTREWQDDQKKVRAFYSGRKAGEKIPQEIKDIFNKRQKQYQEALLTKFGIAKIAQKHARYYNKNVSSNMASVGIGAPVWSSIDKFLYGDGKKVKYKRPGELNSLASNGKSGILLKQDEKGYYLYLSNQKAKAKPLKVYLYDTRTDYDRDMISRKILIVRLVRKAYENGYKYYVQLTVDGAPYERTDEKGEPLCKRGEGKVGLVIDGNNVYAVSDKESRMFKLDENFKEYEKAMEEIETRIEYLRRLNNPNNYEENGTIKRGRLTWNKSCEYKRAAKEKKEMTRKRRETLAITQNVVIKEILEMGNDIYVEKKSYRTKKENYDPDKSDAEYSVKKIRRKKIQNFAPATFLEKLKTKLGDRGALTEISGFGEYFYYCCDSGKSDEERFKNNKVTLDGRVYNAFSYRAVMLKNMENKKLDVEGVKKDLSIIG

SEQ ID NO: 201 - protein, 546 aa, unidentified
  REGION 1..546: note = uncultivated organism
  REGION 1..546: note = MG91B-51 Effector
MANVVYITFGFFRLIMTGLLQPNKNDGIFFQYYQCEVRWAPLAKATTDTCCLTLPLKLEKWHADRLEKRLEIARQIYNTLLRFELKKLHRLEESTEYIDIQNLLKETTDNVERNKLYKQLNKLRQDAGFSEYTFKADIKDFYKHFNNNIGSSVAVHGIAPQVWTAFDRLFYGSGKVVHFKKRGELTSLRGYSVAGKSGGVEIIFRGTYIEWKGLKLPIKLDPKNEYENEMLRQRIKYCRIIKKPGRSKNHWYVQLMLEGKPVIKVNKNTGELLHPVGKGAVGIDIGPQTIAYVSEKEVALKELADQINNIEQEKRVLQRKLDRSRRAMNPGNYEADGTIKRGVKLTRNKSKRYIKTQCELAYLLRRQADIRKRQHNELANHLLSLGDRFYVEDMEWPALAHRAKKTEISEKTGKFKRKKRFGKSIGNKAPSTLIGLLDLKLKSRGLEGVMRVDTKVRASQYNHLTDDYKKKELSERWNNMPDGRRIQRDLYSAFLLQHMNETQTDFDKDALIRDYEQFTVFHDQCIEELRRAPKTLASMGLVRRAS

SEQ ID NO: 202 - protein, 511 aa, unidentified
  REGION 1..511: note = uncultivated organism
  REGION 1..511: note = MG91B-52 Effector
MAKKSATDTCCLTLPLLTEKWQSDRLEKRFEIARQIYNTLLNYELKKLRRLEQSAEYTAIQAAIQKSYEAGKTDKAELKSLFQEREKLRRAAGFSEYDFKTDIKTFYKHFNDNIGSSVAVHGIAPQVWQGFDQLFYGNGKAVHFKRRGDIKSLRGYSVAGKSGGVEIIFRGSYIEWKGLKLSLKLGKNNAYEAEMLQKRVKYCRIVKKAGKHKTHWYVQLMLEGTPAQKCDVNTGELRHPVGRGAVGIDIGPQTIAYAAEAEVALKELADGVKNIEQEKRLLQRKLDRSRRSTNPDNFAADGTIKRGVKLNWHKSKRYIRLQHELAVLQRQQAEVRKRQHTELANHLLSLGDRFYVEDMEWPALTHRAKETAISEKTGRYKRKKRFGKSVANKAPAMLIAILDQKLRSRGLDGVIKVKTSVRASQFNHMTGEYQKKSLAQRWNDMPDGRRIQRDLYSAFLLQHMNAAQDGFDVAALNRDYENFAVLHKRVIEELRTASKTLSSMGITRSVS

SEQ ID NO: 203 - protein, 505 aa, unidentified
  REGION 1..505: note = uncultivated organism
  REGION 1..505: note = MG91B-53 Effector
MGKEEDKEPRCVLTLPLYPEPWQVDIIEKRFHIMERIENSLTAIELRKLKNVERAKKYKDVVAGIKEEKDDKKRSKLWKERNKLLKDAGINQFDFIKDVSLLQKHFASHIAQKVAAAAARTHVWSSFDRVVFGNGRKIHFHKKGDLKSITSGYFGVSMTVKNGYFIWSGGTKDEKDRKVNPITISIRIKQPETYYEKEMLQKQIKYVTIVRKRIKNRYKYYLQFMLVGNPVAKPRTIANGKRVGIDIGTSSAAIVSGKEIKLVELADHIQNNYDKEIQLQRSMDRSRRAMNPQNFNEDGTIKRGIKLIWIESKHYKEMKGQVRELERKNADIRKYQHTCLANYVLSLGTEVYIEQMSFKGLQRKVKETKYDKNGKPKRKKRFGKSIANRAPATFVMILKNKLSGIAGEELHKVNTFTFKASQYDHTNNTYQKKLLGHRWAKLSNGDKIQRDLYSAFLLMNSDTDMQYTDQNKCKRNYKKFKIQHDELIKAMQESSKRYPKSFGIN

SEQ ID NO: 204 - protein, 534 aa, unidentified
  REGION 1..534: note = uncultivated organism
  REGION 1..534: note = MG91B-54 Effector
MGWKSKGRGGKRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRSIS

SEQ ID NO: 205 - protein, 505 aa, unidentified
  REGION 1..505: note = uncultivated organism
  REGION 1..505: note = MG91B-55 Effector
MAKATTDTCCLTLPLKLEKWQADRLEKRLEIARQIYNTLLHFELKKLRRLEESKEYIEIQNLLKETTDHAARKGLFKQLNELRRAAGFSEYTFKSDIKDYYKHFKDNIGSNVAVHGIAPQVWAAFDRLFYGNGKSVHFKQRGELTSLRGYSVAGKSGGVEIIFRETLIEWKGLKLPIKLDSKNAYENEMLQQRVKYCRIVKRPGKTASRWYVQLMLEGRPVLKVDRRTGELRHPVGQGAVGIDIGPQTIAYVADGEVALKELADQLGNIEREKRVLQRRLDRSRRATNPNNYAPDGTIKRGTKLTRNKSKRYLKSQRELSFLLHRQAELRKRQHNELANHLLSLGDRFYVEDMEWPALTHRAKETEISEKTGKYKRKKRFGKSVGNKAPAMLLGLLDIKLKSRGLEGVVKVPTSVRASQYNHLTDDYRKKELGERWNTMPDGRRIQRDLYSAFLLQHMNEARSDFDKTALSADYERFVTLHDQCIEELRRAPKTLASMGLVRRAR

SEQ ID NO: 206 - protein, 396 aa, unidentified
  REGION 1..396: note = uncultivated organism
  REGION 1..396: note = MG91B-56 Effector
MQKHFAAHIATHVAHRAASEVWRAFDKFLFGNGKMVHYVRRGTLRSASNQKAGTTMTLENGYFKWNGGRDQNSIAINIRVAKPTTYYEKEMLQKKAKYFRIVRKWGKIGYKYYLQITLAGLPAVKPRLIANEKKVGIDIGPSSIAIVSDKKVELRALADKVQNNHIEKCSLQRSMDRSRRNLNPQNYNEDGTIRRGIRLNWTFSKHYIEMRGKVRMLERKNVDIRKYQHTCLANEVISLGTDVYIEQMSFKGLQRRAKETTYREDGRPKRKKRFGKSIANRAPAMFVEILGRKLTTIVGTELNKVDTFKFRASQYDHTNGSYKKKLLKERWAQLSNGDRIQRDLYSAFLLKNSNADLNSTNVEQCNATYDVFKIMHDDCITKLKSEKKSCLPSFGV

SEQ ID NO: 207 - protein, 534 aa, unidentified
  REGION 1..534: note = uncultivated organism
  REGION 1..534: note = MG91B-57 Effector
MVRKSKGRGGTLVKKSSTDTCCLTLPLKLEKWQSDRLEKRFEIGRQIYNTVVRAELKKLQQVERTPQYREIEQKLKEAKQEEAKELWKTRTQLLSKSGFSEYGFKSDMKYYYQHFKENIGSSVAVHGIASQVWTAFEKVLFSKDGKMVHFKKKGELSSLRGYSQAGKSGGIEIIFRGSYVEWKGLKLPLKLSPDNDYETEMLSKRVKYVRILRKEGKTKPHWYAQLVLEGKPAIKRDPASGAPKHPVGHGVVGIDIGTRTLAYSTDKEVNLLELADRVQNIEQEKRRLQRKMDRSRRAMNPENYNSDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMLDGERIQRDLYSAFLLQHCKQGLPEYNRKELKKEFGEELYRVFDREALQRDYPQFVAYHHMTIQRLSELPHTIPSMGIRRIIS

SEQ ID NO: 208 - protein, 490 aa, unidentified
  REGION 1..490: note = uncultivated organism
  REGION 1..490: note = MG91B-58 Effector
MARENENSGCFVVTFPLKTEKWQEDRIDKILTLLTVRYREKQKILLDRYIHLSHSPEFKEAKNAGLKALRDFMNEHKFTKYDVEKVFSDSSLSKSVTTDIMLTHGINSSIMQELSHRAWSAWEKKLYGQGKFIKLDNEVNIFKSRLKGSTITGFEYSFTDMTLSMNATKPKKHTMFTIPFVVNRKSEYEAYAMDCEVRNIAIMRQQIRGKNKYYVQFSFAGTPYNKGRKIGTGIVGIDPGPSKIAIVSDSEIRITTLAPSVNRDERAICRMQRKLDRSRRATNPDNYNDDGTIKRGIKLTFEKSNSYKRTQTKLADTQRKLAAKRKIDHNILANEIIAMGNEFHVENNSFKSMQARSKETKKNDKGKNMSKKRFGKSLANRAPSELLTILKNKVNQYADGDYVDIPSGVACTQFDFTNGEFTKHELKERTITTSDGVVHDRDALAAFNMRFVHKKKIEGKKKIEKDKENFDILAMTEAYRQFCSLEKNIK

SEQ ID NO: 209 - protein, 515 aa, unidentified
  REGION 1..515: note = uncultivated organism
  REGION 1..515: note = MG91B-59 Effector
MELPLQVEKYQEDILNKRYEQLRQLYNYAQGKLLRQFRYFEQMKDYQLCKTFKEKKDFFKNHPFQINGILGSNKQPLDITFDEYGINGFVEKLVQKSAGPNITYADLGITTTILDHLSSSIWRAWDKMLYDYHAKRVSFKKYGELDSFSSRRKVVGGKVYFSGLELHLDTMKLSVKLNGKQGSNAKFITMPILYNPKHEDYELWALKGGLESVKVVKVIRRLVRSQYKYYVQLTIEGEKPQKGRTLGKGNVGIDLGPTTVAVSGENIASIDKLASKCDNIQKEITRLARKIDRSRRANNPENFNEDGTIKRGVRLVWNDSKRYKALRKEMAELQRRQAAIRKQQHIDRANALLKEGDTFIVENNQISAWVRKAKETKVNEKTGKIQKKKRFGKSVANHAPAMFVTILENKVKSLGGEVVKVDTQNAASQYDFTNDSFEKHELNERSVTLSNGDTHQRDMLAAFNLQHLTAEKKQYDREAMAQHYDRFCELEQTEIERYKNKEKKDDRSTIGAGGL

SEQ ID NO: 210 - protein, 552 aa, unidentified
  REGION 1..552: note = uncultivated organism
  REGION 1..552: note = MG91B-60 Effector
MDNNIYVLELPLKVEKWQEDIMIKRLNLLRVLYNNILHKYEIRYNQLIKNKKYREAKTKLYNLDAKKEIKEKIKEIEKDLGKKNAVNDERYISLVNELKTISCTLSESDKKIYKDIKTNLEKQFGFTEYGVYGKVNEGIGWNIAQYAKNCGINPTIIETMCKKNIWKSIEGLLFKGKKIHYKKFNEFNSIYGISASKRLTGIGFDGKYVYWRKSGIKNEKGMYSDIVLPINKTINEYEAQALSNEIAYVGIVRKIIRGKNKFYAQLTLKGILPSKIDKSTNLFRKPAVGRVGLDIGTSTLAIVSDSKVELLELADKAQPNEHKNKIIQRKIDRSIRKNNPNKFNEDGTINLSNRDKWIISKNCQKLKLKLRETYRKLSVVRKLQHEELANHILSLGNECYIETMRWKGLQKRSQNDDVNINGKPKKTKRFGKSIGNRAPSQFMTILSNKLNNYGDKLYLVDTIKCRASQYNHMTKEYNKKPLNERWNVMPNGDKIQRDLYSAFLIQNINNTLNGFNEELLNENYVKFKNKHDEEIKRLQSMSNKKLSSMGIK

SEQ ID NO: 211 - protein, 460 aa, unidentified
  REGION 1..460: note = uncultivated organism
  REGION 1..460: note = MG91B-61 Effector
MSELSVVTIPLKTEIWQEDLLAKRMEIYRSIYNAMLGQVLKRYKKMISDEDYISSKKVIEDTYKITDPKEKNSVKKSQKFKDALEMQRNLFREFGFSEFSFKNESLKYAKHFKDITNTAVVTNTVATQMWRAFDKMIFGDGKMVHFKRYGQLMSLTAPARTGIRIVDENKKTVYAMVPGQDYFCLMSTRKGKNILMPLKIDDRDKYIMQMIQRQIHQVRIVRRDSKGKKRYLVQLIVEGTPYIKLDKNGQEKHPVGAKAVGIYIDTTSITLATHDGIEKIDLTMQSDLMKKKDLQRYMDASRRAMNPDNYNEDGTIKKGRMVNGERVPLTWTYSNGYKKASIELKEIYRKEAEKRMIRANIIANKIIAIGSNIVINDYNFRAASRRKADPELDDKMNYISSKRGGSNIANCAPAGIVAKIDTKLKMRGYEGIKKIRINTDSMIENHREVYAKFLLDKAVK

SEQ ID NO: 212 - protein, 552 aa, unidentified
  REGION 1..552: note = uncultivated organism
  REGION 1..552: note = MG91B-62 Effector
MENSIYVLELPLKVEKWQEDIMVKRFNLLRILYNNILHKYELRYNQLIKIKEYREAKTKLYNLDAKKEIKAKIKAIEKELGKKNAINNENYNLLINELKTISCTLSENDKKTYKDIKSNLEKQFGFTEYGIYGKCNEGIGWNIAQYAKNCGINPTIIERMCKKNVWKSIEGFLFKGKKIHYKKYNEFNSLYGTNVSKVLTGISFDGKCVYWKKSGKKNNKGAYSDIVLPINKTVNEYEAQALSNEIAYVGIIRKIIRGKNKFYAQITLKGCLPSKIDKSTNLFRKPAIGRVGLDIGTSTLAIASNSKVELLELADKAQPNENKIKIIQRKIDRSIRKNNPNKFNENGTINISNKNKWVISKNCQKLKLKLKEMYRKLSVIRKLQHEELANHIISLGNECYIETMRWKGLQKRSQNDDINNNGKPKKKKRFGKSISNRAPSQLTTILSNKLNNYGYKLYLIDTTKCRASQYNHITKEYNKKSLNERWNIMPNGDKIQRDLYSAFLIQNINDTLDGFNEKLLNDNYDNFKVMHNKEIERLRLIPKKKLSSMGIK

SEQ ID NO: 213 - protein, 495 aa, unidentified
  REGION 1..495: note = uncultivated organism
  REGION 1..495: note = MG91B-63 Effector
MSKTDGDRCILTLPLHTEPWQEQIIETRFGIMEHLKNSLIAMEMRKLKNLRRTKTFRDIEKRITETPKEERKALNMQRTAMLRDAGFSEFDFINDITPMQKHFADHIAAQIAHRSASDIWRAFESYLYSSGRDVHFQKRGTLDSVACKKIGNGMNYRNDTFEWSGGRTPNARKLYIPVEKPNTEYEKQMLRKEIKNLRIVRKWMKTRFKYYLQLTLVGQPYQKERRIGNGRVGIDIGTRTIAIASDAAVRLLELADQVDGNHAKKCEIQRRMDASKRATNPENYNADGTIRRPVRGRKMTWHFSNNYMKMQSRVRELERKNADIRKYQHNCLANETLALGTDIFVEPMAYKGLQRRAKETKTDENGRYLKKKRFGKSLANKAPSMFLTILDNKLKCHGKQLLKVDIAQFRASQFDHTNGTYRKKLLSERFAELSNGDRIQRDMYSAFLLMNSNDDLTGPDPERCAATYERFKELHEKETVRIRNKQGRRIASFGI

SEQ ID NO: 214 - protein, 511 aa, unidentified
  REGION 1..511: note = uncultivated organism
  REGION 1..511: note = MG91B-64 Effector
MGKSKTETCCLTLPLKLEKWQEDRLAKRFEIARQIYNTLVHAELKKLNQIERSSEYQTIQDQITNLDRNDPERIKELPSLYKKRTKLLRDAGFTEYGFKSDVKYYYKYFHKNIGSSVAVHGIAPQVWAAFEKKLFKKQGKIVHYKRKGTLRSVKGGSATGKSGGTEIIFRGTYIQWKDLNLPLKQSPDNAYESEMLGYRVKYVRILRKPGKSKDRWYAQLVLEGKPVIKRNPMTGAALHPVGHGAVGLDIGPQTLAYSAADGVGLLELADQVQNIERQKALLQRKLDRSRRASNPDNYNEDGTIKRGVKLTRNQSKRYCKTQRDLAYLQHCQAETRKRQHIALANHLLTLGDRFYVEQMSWAALARRSKKTEVSEKTGKIKRKKRFGKSIANKAPAMLIEILRQKCISLGLPGVVDVPTSIKASQYNHQSKDYIKKNLSERWNDMPDGEKIQRDLYSAFLLQHCTPELNAFDQESLQQDYPQFVQYHHQVMQQLSEMPKTPSSMGIRRRTS

SEQ ID NO: 215 - protein, 490 aa, unidentified
  REGION 1..490: note = uncultivated organism
  REGION 1..490: note = MG91B-65 Effector
MAHKENQKAFVVTFPLRTEKWQEDRIDKIMRMLTTFYNDRQRKLVRRYIYLSHSKAYKEAKSKGVVAFKNYMKENGFSLYGLDAFFKADTKGALYQCGLNSMFLQYLSQCAWSAWDKKLFGNGDFVKTTKVVNIFCSRNLKGKFCGFDYDLSTFTIRIKSTCTKKIICTIPFVVNEKSYYELFALSQKICRIGIVRKLIRSKYKYYVQFTFDGIPYNKGRNIGTGIVGIDPGPSKIAIVGDNKVEILKLAPNIEEDERKTARLKRKLDRSRRATNPHMYKEDGTIIKGQRQTCFSKAYNETRKQLADAQRKLAAKRKIAHNELANTLLEYGDTFKVEANSYKSMQARAKATSVTKSGRIRSKKRYGKSIKNRAPSEFLVILKNKLLYYPKGKYYDVPASYACTQFDFTNESFTEHKISERKIVTSDGIQHNRDTLAAFNIKNSIVNDTSTKKKKITKSKEFFDIKKMKASYNEFCIMEHTSFQYKEIMEL

SEQ ID NO: 216 - protein, 537 aa, unidentified
  REGION 1..537: note = uncultivated organism
  REGION 1..537: note = MG91B-66 Effector
MISDEEYKKLLKQFHKLSDRHILVVETDMPYSDVLKVVILSDKIRKAGNELVSFMRKNYDQLIRTKRYRKLLKLYGSTKDKKKCKDLADQLNEMQKAYNVTWDYCRKSMIPIGKKYGIDAVFALTKAEDVWRGIEKCLYGNGKAVHFSKYGELPCIRAKQINRGIPISVKDDRLQFKLGKTAFGIQVNDRFQQDEVDAVLTYLAESEIVDNKAVNTLMEDAYCIDTYRPCYATLVPKMIRGRYRIYLHLTIEGKAKPKYDKYGNLRHKYGKGVIGADIGTQTVAYTSDTEVGLKNLSERGNSIQTSERKERLLYRAMDRSRRAANPQNYNEDGTVKKGRKTWKYSNHYKKLKAKHSELCRINAVNRQLAINEDANHLRSLGDTFVTEPKNASKLMKRAKETTVNSKGKFNRKKRFGKSIKNRCPSGFQTTVEKKFKTTGGTYIEVPNNYRASQYDHTVDDYIKKKLSDRLYKLQDGTEVQRDWYSSFLLYCYDYKIQKIDKNKCITQFDNCYKKEKALITWIKAHKIKVLNSGIKIA

SEQ ID NO: 217 - protein, 535 aa, unidentified
  REGION 1..535: note = uncultivated organism
  REGION 1..535: note = MG91B-67 Effector
MATENFVLELPLKVEKWQADILNKRYEYLRQIYNYVQRKLIRQYRYFEQMDEFRQCTTYKEKREFFKSHPFYFKDIVGRNGEPFAIKFPYSMSASSGSKMDGFSGYIAKLGKLPAGANKSYSDLGINSFMLERLASNMWAAWNKYLYDRDSKRVSFKKYRDLNTFGSRKANNTTFIGMEIHLDSMEVIIKTNGKLGKYAKYITLPIDYKHSMTEYEMFALKGGFDSIKILNVVRKEIRGREKYYVQLTIEGEKPQKGRMLGKGNVGIDLGPSTIAVSSRQKVSIDKLANKCDDIQRDLYVVSRKMDRSRKANNPQNFNEDGTIKPISRKNGERRYWNDSKRYERLRSERKELLRKQAAIRKLQHIETANALLELGDTFIVENNPVSGWTRKSKETKLNKKGRIQSKKRFGKSVANHAPAMFSTILENKVKSLGGQFKKVDVKNAASRFDFTNGEFTEHKLNERVITLSNGDKHQRDLMAAFNLQHLNLDGDLSKDYDVEMMQRDYPDFCRLEREEMDMYLQGIKKNERTTIGAFK

SEQ ID NO: 218 - protein, 496 aa, unidentified
  REGION 1..496: note = uncultivated organism
  REGION 1..496: note = MG91B-68 Effector
MAKNEQNCCVLTLPLLTEPWQEHIIETRFRIMEQLKNALIAKELGKLNNLKRTREWKRLMKSIQDAKDEKEKKQLFKKRTELLRNVGFSDFSFSNDVTNLKIHKHFAEHINSGIAQRAGKDVWRAFEKYLFSNGKTVHFHRRGTLDSVANRYAREGMMIDGDILTWKGGQSDHQIVLKIRIRRLKNAYEREMLQKTVRNLRIVRKWMKTRYKYYLQITLQGDPASKYRSVGKGRIGIDIGTQTIAWSSPTEVRLAELAAGIKRNHAEKTALQRKMDRSRRTANPQNYNPDGTIRRGVKLRWVKTKHYLRMEGRVRELERKNADIRQYEHICMANHILSLGNEVYIEDMSFKGLQGRAKETKLDKNGRFARKKRFGKSLANRAPSMFVTILENKLKASGGSLHKVNTRTFRASQYDHTADEFKKKTLGQRWNHLKNGDKIQRDLYSAFLLMNAAPSLEEPDKKLCGKTYPQFNRLHDAEMERLRAEEKPHLSSMGIH

SEQ ID NO: 219 - protein, 540 aa, unidentified
  REGION 1..540: note = uncultivated organism
  REGION 1..540: note = MG91B-69 Effector
MMYNTKYQAKRGDPVGEKNSNDTDTCVLTLPLRVEKWQEDCLLKRFEAARRIYNSLLNFELKKLRQLERTVEYRQIQSVISSAYLSNTKDSLECKNAFKRREKLISANGISENGFKSDVKFFYKHFDGLIGSSVAVHCIAVQLWAAFDKYLSGSGKEIHFKRLGDISSLKGYSVKGKSGGLEIIFRGQYVEWTRKKKKLLLPLKIDPDNEYEREMLSRRVKYVRIIKKPGKNRLRWYAQLTLEGKPALKYSAGTGKPIHSVGKGNVGIDIGPQTVAYASDKEVALLELADRVTNIEHQKKKLQRKLDRSRRATNPDNYKPDGTIKRGVKLTRNKSKRYLKLQSKLAYIQHRQAEIRKLQHTCLANHMLTLGDRFFVEKMNWPGLAHRAKETSISEKTGRYKRKKRFGKSLANKAPAMFISILKRKCETLGLPGVVEVDTYNLKASQYNHLSDTYEKKSLSERWNYMPISDGSDSSKTKRIQRDLYSAFLLQHTIPQPCSFDKEALRIDYPNFVTMHDAEIHRLESLPKTITSMGVRRSHS

SEQ ID NO: 220 - protein, 511 aa, unidentified
  REGION 1..511: note = uncultivated organism
  REGION 1..511: note = MG91B-70 Effector
MREDSQNTCCLTLPLKIEKWQADRLIKRFEIARQIYNTLVHAELKKLQKMEASAEYRQIQDRIRELNPSDVRAKKEWKILKDKQEKLWKAWKITEYGFKSDIKAYYKYFNENIGSAVATHDIAPRVWAAFEKKLNDRTGKVKIQYKKAGDIRSLKGFSVSGKSGGTEIIFHKTYIAWKGLTLPLKLSPDNAYEKEMLMRRIKYVRILRKPGKHKDRWYAQLSLEGNPVVKLDPVSGLPRHPIGKGAVGIDIGPQTLAYSAEGGADLVELADQVQCIEQEKSRLQRKMDRSRRASNPDNYAQDGTIKRGVKLTHNKSRRYCKLQKELAYMLYRQAETRKRQHRELINRLLTMGDCFYIEDMHWNSLAHRAKKTEISEKTGRYKRKKRFGKSIANKAPAMLVDMLEQKCKSLELDVVMVNTSLKASQYNHQTGKYEKKTLSQRWNYMPDGEQIQRDLYSAFLLQHVVPEKNAFDNEALCRDYPKFVQYHHEVIERLRSLPKTISSMGIRQSVS

SEQ ID NO: 221 - protein, 483 aa, unidentified
  REGION 1..483: note = uncultivated organism
  REGION 1..483: note = MG91B-71 Effector
MGDKCSVLELKINVRKEQYHRLDVKFEDARAVYNAVLNEALSNYEQITRTKEYRADQAVIKKYYEDKKNGTVKKMPDEVRAIFDKRKKQFQDAKLTKFGLINIAGYHKNHYAKNVSSVIANKIGEMAWTAMNQLIYGNGKLVKFKKPGEMTSIASDNKSGIRLMVDNGKYYLLLSNRAQKAKPMILSIKQPHNEYELDMITRPIKIVRIVRRKNGNRFNYFVQLTVEGTPPQKLKKDGSRVHQIREGKVGVYIKDDSVYAVADDGRIAHWHFCKNQQKYEEKKADYDRELSKLRQLNNPDNYDENGVIKKGSKKWHYSIKYKKILLQRSYLIHKNTESRNITRYVIANEILAMGNDIYILKKDYRTKKEKFDEDNRKSNEQYRQLKDRRKNIQNFAYADLISKIKSKIDSDNSGAVNEVTVSKDLYYYVHDEDKVNKSRFVDGIIAFKNRTCLEESYRALLIMNYDGKNYNRDMVEKGMSWIC

SEQ ID NO: 222 - protein, 508 aa, unidentified
  REGION 1..508: note = uncultivated organism
  REGION 1..508: note = MG91B-72 Effector
MADKECCVLTLPLQTENWQEHIIEKRFAIMEHLKNQLIAKELRRLKNLERTKEYRILIDEINNTPKEKRKALFSKRRKLLKDSGFNRNKFKDDMAVMQKHFEEHIATQIAQEAASDVWRSFDKYLFNNGKYIHFTRKGSLSSINCQKINNGMKYIDGYFIWTCGICSGKRDRRIILKIKVTPPKNEYEKEMLSKKIKYLRIVKKWVKTRYKYYLQFALEGKPVKKDRIVNKGNVGIDIGTQTVAISSADSVKLLELADKIDKNHTKKKILQRKMDRSRRYTNPQNYNEDGTIKKPPKGQRYCWYKSKKYIQLAGKVRELERKNADIRKYQHTCLANWILSLGDTVYVEQMNFSGLQRRAKETKIDKNGKYAKKKRYGKSLANKAPSMFLTILESKLNQYGGQLNKINTYEFKASQYDHTDDTFTKHNRSERWHILSNGDKNQRDLYSAFLIMNSDISLEHCNREKCNETYSNFKVLQDKEIERLYSDIRKGNCKNISSFGFQRNAKAM

SEQ ID NO: 223 - protein, 485 aa, unidentified
  REGION 1..485: note = uncultivated organism
  REGION 1..485: note = MG91B-73 Effector
MANFIVEFPLKTEKYQEDILNKRFEIGRKIYNSLVNITQKRYKEMIKTKKYRKLISSLTGNKKTDKEIWKQINDIRKQYGMSEYSFHEDVKQIQKHFKDNIDSFTAQKIATTLWKSYDKLFFKNGKKIYFKCYGELNSLEGKSNKTGIRLIGDMLIWNKLKIPVMIDYDNYYEYQAMQCDICYNRIVRKYVRNKYKFYVQVVFKGTPPIKADATGKTKHCIGEGDVGLDIGTSTIAVASQSDVKILELADRVQNIENQKQRLLRKIDRSRRFTNFNNYNADGTINKQGNKKLKWNKSSHYIKYQNELKELYRKQADIRKYQHECLANYIISLGNKVYVEKMNFAGLQKRAINTEKNDKGKFKRKKRFGKSLANKAPSMLLTIINRKLAYYGEKLIEIDTINAKASQFNHFDGTYTKKTLSQRWNDFNGIKIQRDMYSAFLIMNIADDLKSFDIDKCNKRFENFYRLHSLEVNRLVGKKNLSSIAI

SEQ ID NO: 224 - protein, 524 aa, unidentified
  REGION 1..524: note = uncultivated organism
  REGION 1..524: note = MG91B-74 Effector
MEEVFVLELPLQVEKYQADILNKRYEQLRQLYNYVQGKLLRQYRYFEQMTEFQACKKIKEKRDFFRSHPFQINETLKGNNTPFKVSFDEYSIKGFVEKLVQKSVGPNMTYADLGFTTYILDHLGTSVWSAWNKKLYDFKAKHISFKKHGELDSFSSRRKVVRGVVYFSGMELHLDTMELSIKLNGKVGKEAKFITMSILHNPKHAEYEMWALKGGLESVKVVKVVRRLVRGQYKYYAQLTIEGEKPQKGRTLGKGNVGIDIGPTTVAVSGENIASIDKLASKCDNIQEEITRLARKIDRSRRANNPENFNEDGTIKRGVRLVWNDSKHYKELRRELAELQRRQAAIRKQQHIDRANELLKEGDTFIVENNQISGWTRKAKETKVNEKTGKIQKKKRFGKSIANHAPAMFVTILENKVKSLGGEVVKVDTQNAASQYDFTNNSFEKHELKERSVILSNGDTHQRDMLAAFNLQHLKYDAPEKKQYDTEAMTQHYDRFCELEQAEIQRYKNKEKKDDRSTIGAGDL

SEQ ID NO: 225 - protein, 511 aa, unidentified
  REGION 1..511: note = uncultivated organism
  REGION 1..511: note = MG91B-75 Effector
MAQRHSTPTCCLTLPLQLEKWQEDRLIKRFEIAGKIYNALLNAELKKLHRLEQLPEYKEIQSQLKAYYQNNLPKTAAFKELNNRRSKLLSEHGFSEYAFKGDIKKFYKHFSSNIGSSVATHCIASQVWSAFEKRLFGGGKFVHFKKSGDIRSLKGFSSSRKSGGVEIMFRGDHIEWKGLLLRLKLDPDNQYESEMLSRRIKYVRIIRKPGKAKWRWYAQLSVEGLPAIKRNDETGEPVHPIGRGAVGIDIGPQTIAYVSQTEVALKELADRVQNIEHEKRLIQRKLDRSRRATNPANYADNGTIKRGVKLTRNKSKRYLKLQMELSFLQHKQAEIRKQQHINLANHLLSLGDCFYVEDMAWSSLARKAKETAVSEKTGKYKRKKRFGKSIANKAPASLISILDQKCTSLGLLGVIKVPTSVRASQYNHITKSYEKKTLSQRWNIMPDGRRIQRDIYSAFLLQHVTPQRDGFSDEALQQDYENFVSLHDSVIQQLRLMPKTIASMGIRRTYS

SEQ ID NO: 226 - protein, 536 aa, unidentified
  REGION 1..536: note = uncultivated organism
  REGION 1..536: note = MG91B-76 Effector
MNEKEYKKSLRQFHKHSDRHILVVETDMSFSDIQKVVALSDKIRKAGNELVGLMIKNYDQLIRTKRYRKVRKLYGATEEKKKRKVLASQLNEMQKQYHVTWDDCRTSMIPIGKKYGIDAVFALTKTEDVWRGIEKCLYANGKTLHFSKYGVLPCIRAKQMNRGIPISVKENQLQFKFGESTFGIQRKDRFQSDEIHAVLDYLAEPEIIDKKAIQTFVEKAYCINTYRPCYATLVPKLIRGKYRVYLHLTIEGKAKPKQDRFGNPRHKFGKGIIGADIGTQTVAYTSDTEVGLKNLSERGNSIQKSERLERLYYRAMDRSRRATNPQNYNEDGTIKKGRKSWKYSNHYKRLKAKHAELCRINARNRQLAINEDANSLRSLGDVLITEPKNASKLMKRAKETTKNKKGKFNKKKRFGKSIKSRCPGGFQSTIEKKFEVSGGRYIEVPGNYRASQYDHTADTYVKKKLSDRMYHLADGTLVQRDWYSSFLLYCYDDKIEEINKTKCRTEFEKHYKKEKALIEWIQANQIKILNSGIKTA

SEQ ID NO: 227 - protein, 535 aa, unidentified
  REGION 1..535: note = uncultivated organism
  REGION 1..535: note = MG91B-77 Effector
MAQETFVLELPLKVEKWQADILDKRYEYLRQIYNYVQKKLIRQYKYFEQMNEFRQCTTYREKREFFRSHPFYYNEVVGRNGEPFAIKFPYSMSESNGAKLDGFNGYISKLGKLPAGANKTYADLGINSFMLERLASNMWSAWNKFLYDRDAKRVAFKKYGELNTFGSRKKNNISFIGMTIHLDTMELVIKTNGRTGRFAEYITLPIGAGKPLTEYEMYALKGGFDSIKILNVVRKQIRGNNKYYLQMTIEGEKPQKGRTLGVGCVGIDLGPSTIAVSSLQKVSIDKLANKCDDIQRDLNRVSRKMDRSRRANNPQNFNEDGTIKPVSRKNGERRVWNDSKRYRKLREERRELLRKQAAIRKAQHIETANALLPLGDTFIVENNPVSSWTRKSKETKINKKGKIQSKKRFGKSVANHAPSMFATILQNKVESLGGEFRKVEVKNAASRFDFTNGEFTEHGLGERVIALSNGDKHQRDLLAAFNLQHLNADGDTTKDYDVATMQEDYPTFCRLEREEMDLYLQGKKKNERTTIGAFK

SEQ ID NO: 228 - protein, 521 aa, unidentified
  REGION 1..521: note = uncultivated organism
  REGION 1..521: note = MG91B-78 Effector
MAKEVFVIEFPLKVEKWQADILNKRYEHLRRLYNYVQSKLLRQYIYFTQMQDYKKCKTLKEKKNFFKEHVFHIKGILGRNKSPLDITFTEYGILGMVTKLSQLSMGGKSYSDIGIGFTILANLADNIWAAWNKRLYDFECERISFKKYGSLDTFGSGKINNKYFCGFDLHLDRMTLDIKINGRTGKQAKFISLPIRYNMKHVDYEMNALAGGFDSIKVLTVVRRMVRGQYKYYLQMTIEGVKPQKGRHLGKGRVGIDIGPTTIAVSSNSTVSIDKLAAKCDNIEKDIQRINRKLDRSRRTMNPDNFNEDGTIKRGVRLIWNNSENYKALRRQKAELQRKQAAVRKMQHIERANSLLSEGDTFIVENNPIKGWAAKAKETKVSEKTGKIQKKKRWGKSVANHSPAMFVTILENKVKSMGGTVIKADVKNAASRYDFTDGSFTKHDLDERGVTLSNGDTHQRDMLAAFNLQHLIYDAEKPKQYDQLRMKSHYPRFCELEQDEIERYRNKVKKDDRCTIAASRL

SEQ ID NO: 229 - protein, 505 aa, unidentified
  REGION 1..505: note = uncultivated organism
  REGION 1..505: note = MG91B-79 Effector
MARSTSDTCCLTLPLKIEKWQADRLEKRLEIARQIYNTLLRFELKKLKQMEAREDYLAIQEQLKETKNDAERKKLYKQIEKIRSETGFSEYSFKTDIKAFYKHFQENIGSSVAVHGIASQVWIAFDKLFHGSGKAVHFKKRGELTSVRGYSVAGKSGGIEIIFRNTFIEWKGLKLPIKLDPSNAYETEMLQKRVKYCRIIKKPGKNKNHWYVQLSLEGKPAVKYHQQTGEILHPVGHGNVGIDIGPQTIAYVADGEIELKELADQVNNIERKKRVLLRKMDRSRRATNPDNYNPDGTIKRGVKLTKNKSKRYIKCQRQLAHLMRQQAETRKRQHNELANHLLSLGDHFYIEDMEWPSLTHRSTKTEISEKTGKFKRKKRFGKSVGNKAPATLTTLLDLKLKSRGLEGVTKVNTMVRASQYNHITDDYQKKKLSDRWNNMPDGRKIQRDLYSAFLLQHMNETHDGFDKAALCRDYEQFTVLHDQCIKRLEQLKKTIASMGIVRKPS

SEQ ID NO: 230 - protein, 609 aa, unidentified
  REGION 1..609: note = uncultivated organism
  REGION 1..609: note = MG91B-80 Effector
MAKENFVLTLPLKVEKCHADILDKRYELLRQTYNMVQRKLLRQYIYFAQQKEYKACENFQQRRDFFASHPFHFKGINSKNGEPADIKFPFTYESRSSNSGKRPSGGISDYVSKLWAHRVGADKTLANLGINSYILEELGLHIMKAWEKRLYDPKSSRVSFKDYGGVNSFGSRYKKGNFAGFTINLEEGFITYNANGRQGRFADLIKLPIDFSREKKDYAAYALKGGIDSLRKLIILRKNIRGRNKYYLQLTISGEKPQKGRALGQGNVGIDIGPSTIATSSLQGVHFDKLADKCDDIEHELYLINRKMARSRYENNRQNYNADKTIKRGVRLVWKKDKDVKRYASLKLKRRELYRRQAEIRKLQHLEMANAMLSQGDTFIVENNPIDAWVRRAKETTKTKTGRNRCKKRYGKSVANHAPAMFVTILKNKVESLGGKFYEVDVRYAATQFDFTNGVKTKHEVSERSITLSNGHIHQRDMLSAFNLQHIKLESIGTDDKEYNIEQMKADYPIFCQLETAEQQRFKEARHNERDAQYFVGAQEKQLRHQPQDESVANRHNGGVGRKHCNTRLEGENAKITGSGLVRESIERVSQVPDEYHPIGDQPIILDGL

SEQ ID NO: 231 - protein, 527 aa, unidentified
  REGION 1..527: note = uncultivated organism
  REGION 1..527: note = MG91B-81 Effector
MNEVFVLELPLQVEKYQADILDKRYEQLRQLYNYVQGKLFRQYRYFEQMKEYQACEKINEKKDFFRTHPFLINETLKGIKTPFMVSFDEYSIKGFIEKLAQKQVGPNMTFADLGFTTYILDHLGASVWAAWDKKLYDFKAKRISFKKYGELDSFSSRRKVVRGVVYFSGMELHLDTMELAIKMNGKVGKEAKFITLKMLHNPKHADYEMWALKGGVEIVKVVKVVRRLVRSQYKYYVQLTIEGEKPQKGRTLGKGNVGIDLGPTTVAVCGENVVSIDKLASKCDNIQEKITRLSRKIDRSRRANNPQNFNEDGTIKRGIKLVWKKDKDLKRYSKLKRELVELRRKQTAIRKQQHIDRANALLKEGDTFIVENNQISSWTMRAKETKVNEKTGRIQKKKRFGKSVANHAPSMFINILENKVKSLGGKVVKVDTKNAASQYDFTDGSFTKHELKERNVTLSNGDTHQRDMLAAFNLQHLKYNEKETKQYDTEAMAADYERFCQLEQEEIQRYKNKEKKDDRGTIGAEKL

SEQ ID NO: 232 - protein, 609 aa, unidentified
  REGION 1..609: note = uncultivated organism
  REGION 1..609: note = MG91B-82 Effector
MAKENFVLTLPLKVEKWQADILDKRYELLRQVYNMVRQKLLRQYIYFSQQKAYKECGKDTKKKGKFFAEHPFFIKIDGKPCEVKFPTWKKSNQTASNSIAKYVSKLSSYKLGQDKTLSDFGINSYILEELGARIMKAWEDRLYKFKAKHISKREYGDVNSFSTRIKKSDNKISYPGFTINLEEGYITYNTNGRRGGFANLIKLPIDFSREKKGYAVEALKGGLESIREISVVRKYIRGKNKYYLQLTIDGEKPQKGRTLGRGNVGIDIGPSTIATSSLMGVHFDKLADKCDDIEHELYLVNRKLDRSRRATNPQNYNTDGTFKTIKNRERRVWNYSNRYNKLKLVRKELFRRQAEIRKQQHLAMANALLSQGDTFIVENNPIDAWVRRAKETSKNKKGNYRCKKRYGKSVAHHAPAMFVSILENKVKSLGGELHKVDVGYAATQFDFTNGEFTKHAVNERSITLSNGHTHQRDMLSAFNLQHARLKTDSRNIEKTVDNYDIEQMNADYPIFCRLETAEQERFKAIRHYERDALFFVGVSNKHLRCQPQEETVANRHNGGVGRKDCNISLGSENAEITGNGLIRESIARVSQVPDECHPFAGKPIVLDGL

SEQ ID NO: 233 - protein, 527 aa, unidentified
  REGION 1..527: note = uncultivated organism
  REGION 1..527: note = MG91B-83 Effector
MEEVFVLELPLQVEKYQADILNKRYEQLRQLYNYVQGKLLRQYKYFEQMKEYEVCKSIREKRDFFRNHPFHITGVLDKSGEPLDITFDEYGIQGFVEKIIHKSVGANTTYADLGFNTLILDYLSASIWSAWDKKLYDFKAKRISFKKYGELDSFSSRRKVVRGVVYFSGMELHLDTMELAIKMNGKVGKEAKFITLKMLHNPKHADYEMWALKGGVDSVKVVKVVRRLVRSQYKYYVQLTIEGEKPQKGRTLGKGNVGIDLGPTTVAISGKNVVSIDKLASKCDNIQEEITRLSRKIDRSRRANNPQNFNEDGTIKRGVKLVWNDSKRYKELRRELAELRRKQAAIRKQQHIDRANALLKEGDTFIVENNQISGWTTRAKETKVNEKTGKIQKKKRFGKSVANHAPSMFVSILENKVKSLGGEVVKVDTKNAASQYDFTDGSFTKHELKERNVTLSNGNTHQRDMLAAFNLQHLKYDEAEAKQYDTDAMAADYERFCQLEQEEIQRYKNKEKKDDRGTIGVEQLLKT

SEQ ID NO: 234 - protein, 525 aa, unidentified
  REGION 1..525: note = uncultivated organism
  REGION 1..525: note = MG91B-84 Effector
MTEVFVLELPLQVEKYQADILNKRYEQLRQLYNYVQGKLLKQYRYFEQTKKFRACKTIKEKRDFFKKHPFHINGILGSNKELLDITFDEYGISGFVTKLGMKNIGSNISYTDVGINSSILAKLSSNIWSSWIKFLYDSKAKRIAFKKYGELNSFGCGKKTNGSRKKIFSGMELNLERMELSIKLNGKVGKSAKFIILPILHNPKHEDYEMMALKGGFDSVKVITVVRRMVRGHYKYYLQMSIEGEKPQKGRTLGKGNVGIDLGPTTVAVSGENIASIDKLAPKCDNIEAAKVKLSRKIDRSRRANNPLNFNKDGTVKRGVRLVWNDSNRYKALRCEMAELHRKQAAIRKQQHIDRANALLKEGDTFIVENNQISGWTRRAKETKVDETTGKIRKKKRFGKSVANHAPSMFVTILENKVKSLGGQVVKVDTQNAASQYDFTNDSFEKHELKERSVTLSNGDVHQRDMLAAFNLQHLKYDETEKKKYNRKAMEQHYERFCELEKEEIRRYKEKEKADDRSTIGTSNF

SEQ ID NO: 235 - protein, 594 aa, unidentified
  REGION 1..594: note = uncultivated organism
  REGION 1..594: note = MG91B-85 Effector
MVDLKKQLCSSASNSENQGVRFQTVVLYTKFVHKSVQLSPESSVFAVALFVRMMYNTKYQAKRGDPVGEKKNNNNNTDTCVLTLPLRVEKWQEDCLLKRFEAARRIYNSLLNFELKKLRQLERTVEYRQIQSVISSAYRSNTKDSPECKAALKRREKLIDENGLTKNGFKNDVKFFYKHFDGLIGSSVAVHCIAVQLWAAFNKYLCGNGKEIHFKRLGDISSLKGYSVKGKSGGCEIIFRGQYVEWTRKKKKLLLPLKIDPDNEYEREMLSRRVKYVRIIKKPGKNRLRWYAQLTLEGKPALKYSAETGKPIHSVGKGDVGIDIGPQTVAYASDKEVALLELADRVTNIEHQKKKLQRKLDRSRRATNPDNYKPDGTIKRGVKLTRNKSKRYLKLQSKLAYIQHRQAEIRKLQHTCLANHMLTLGDRFFVEKMNWPGLAHRAKETSISEKTGRYKRKKRFGKSLANKAPAMFISILKQKCETLGLPGVVEVDTFRLKASQYNHLSDTCEKKSLSERWNYMPISDGSDSSKTKRIQRDLYSAFLLQHTIPQSCSFDKEALRIDYPNFVTMHDAEIHRLESLPKTITSMGVRRSHS

SEQ ID NO: 236 - protein, 527 aa, unidentified
  REGION 1..527: note = uncultivated organism
  REGION 1..527: note = MG91B-86 Effector
MPTENFVLELPLRVEKYQADILDKRYEQLRQLYNYVQGKLLRQYRYFEQMKAYQDCNSIKEKREFFSSHPFHINGIYGRNKDLLDITFSKFGIFGLVTKLGQKSVGSGYTYTDCGISSTILAHMSANIWSAWNKMLNDYHTKRISFKRHGELNSFGSGKIDNEYFPGLVLRLEKMELDIKLNGKTGKNAKFITLSILHNPKHADYEMMALKGGFDSVKVITVVRRMVRGRYKYYLQMSIEGEKPKKGRTLGKGTVGIDLGPSTIAVSGNNIASIDKLASKCDNIEKKIQKLNRKIDRSRRANNPDNYNEDGTYKRGVRLSRNESNRYKALRKEKAELQRRQAAIRKLQHIERANELLKEGDTFVVENNQFKGMAKRSKETKVDEKTGKMRSKKRSGKSIANHAPAMFVTILKNKVESLGGRFEIVNTNNAASQYDFTDGSFTKHELSERNITLSNGDTHQRDMLAAFNLEHLKYEAKDPKKYDSEAMHEHYERFCQLEREEIERYKNKEKKDDRSTISASTIVYDKK

SEQ ID NO: 237 - protein, 579 aa, unidentified
  REGION 1..579: note = uncultivated organism
  REGION 1..579: note = MG91B-87 Effector
MAFGNKNSTPSFVLTLPIKVTKQDEIFLSKKFRVGCTVYNQMVTKTTKMWHQLRKTREYKNLIKAIKAAPANSDERKALLVQRSNLIKQAGFSEGAFHKLVVPYQKAYNVNCDVAQKVASAVWKAWGDFFYGKGKTVHYKRLDDFVTLSGKKNTNGIVFRPANHTVSSLESAKRKAKSSVEKKYFDAYRKLNAKKDEEVVLPDEVKAQMDKEVAAAVAKIKPCIGDGNLRIIYGKHEFLVKLRNPDTQTGRYQQEALKCGVKYCRIVRSWVGTKWKYYAQLVLEGYPPIKCDSNGVAKHPVEQGRIGVDIGTQTIAFCGKDVCDLRVLAPSAVVEARNGLTKEIARVMRQMDRSRRAMNPQYFNENGTVKRLKCKNGHKQIRHWNYSKNYYRLLHKLRDLNRKLAAVRKTEHYILANELLTYGNEFVVEDMNYKALQKRSKKTKINPKTGRARSKKRFGKSIGRCAPAMFISILNQKANRYGGSVIKVSTFETKASQFDHTDDSYTKKKLSQRFAKLSDGTVVQRDMYSAFLLLHLRKNLQSYNKKTIKKDFPLFKKLHDETKERLQNSHEWLPASVGF

SEQ ID NO: 238 - protein, 525 aa, unidentified
  REGION 1..525: note = uncultivated organism
  REGION 1..525: note = MG91B-88 Effector
MNSDTFIMELPLIVEKFQSDELDKRFEILRRIYNMAQNKLERQFCYLSKFKEYQECTTKKAKGEFIKKHPFSFKDILDKSGNPLMITFTKYDIQGYLSKFAKLKIDSQYTYSDMGINTSMLMHMGMAIWSAWEKVIFDSKADKISYKKYGDLKSIGVDWEPKHPAGFILNLRKGELKLNLNNRQGQYARYITMKIRFGKKEHQVWYEGDALNAGEDSIRVLTIVRRKIRGRYNYYLQMTIRNNKPQKGRTLGEGRVAIDLGTSKIAVVAKDKVHLLPLSPSILSVSDELRIIQRQMDRSRRANNPHKYHEDGTVKKGNHDKWVNSKEYIRLRNMKAELERKQAAVRKQDHIALANYLLSLGNEFVIEDNDVQEWAKRKKEEKRRKSDGKNLSKAGLGKQIGNHAPSMFVTILKNKVESLGGTFVKVDNNNRATDYDFTNNTFNEHKLEERYITLGNGNRHQRDTLAAFNLYHLDTAPEKEKCYNLYEMRKHYAQFCEFEKEALLRWKGMTPKREKMKVREHERGL

SEQ ID NO: 239 - protein, 524 aa, unidentified
  REGION 1..524: note = uncultivated organism
  REGION 1..524: note = MG91B-89 Effector
MMKEVFVLELPLLVEKYQADILNKRYEQLRQLYNYVQGKLLRQYRYFEQMVEFQACKTIREKRDFFRSHPFHVNGRLAGDKELPEITFDEYGIKGFVEKLAQKSVGPNMTYADLGFTTYILDHLGANIWSAWDKKLYDYRAKRISFKKFGELDSFSSRRKVVGGKVYFSGMELRLDTMELAIKLNAKQGKNAKFVTLRILHNPKHADYEMWALNGGLDSIKVVKVVRRLVRGQYKYYVQLSIEGEKPQKGRTLGKGNVGIDIGPTTVAVSGENIASIDKLASKCDNIQEEITRLARKIDRSRRANNPENFNEDGTVKRGVRLVWKDSKRYKELRKEMAELQRRQAAIRKQQHIDRANALLKEGDTFIVENNPIKGWAARKKETKIDEKTGKYKSKKGRGKSIKNHAPAMFVTILENKVKSLGGKVERVDTQNAASRFDFTDGSFTDHGYDRKVTLSNGDTHQRDMLAAFNLQHLKYDEAEKKQYDTKAMAQHYDRFCELEQEEIQRYKNKEKKDDRSTIGAGEL

SEQ ID NO: 240 - protein, 512 aa, unidentified
  REGION 1..512: note = uncultivated organism
  REGION 1..512: note = MG91B-90 Effector
MNEVFVLELPLQVEKYQADILNKRYEQLRQLYNYVQGKLLRQYRYFEQMKEYQACKTIKEKRDFFKSHPFQVNESLGGDKSAFEISFNEFSIKGFIEKLVQKSVGANMTYADLGFTTNILDHLGASVWAAWDKKLYDFKAKRISFKKYGELDSFSSRRKVVGGKVYFSGMELHLDTMELAIKMNGKQGNNAKFITLKMQHNPKHAEYEMWALKGGVDSVKVVKVVRRLVRSQYKYYVQLTIEGEKPQKGRTLGMGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISSWTTRAKETKVNEKTGKIQKKKRFGKSVANHAPSMFVSILENKVKSLGGEVVKVDTKNAASQYDFTDGSFTKHELKERNVTLSNGDTHQRDMLAAFNLQHLKYDEKDTKQYDTEAMTADYERFCQFEQEEILLYNNKLN

SEQ ID NO: 241 - protein, 511 aa, unidentified
  REGION 1..511: note = uncultivated organism
  REGION 1..511: note = MG91B-91 Effector
MSKRNTTDTCCLTLPLKLEKWQEDRLSKRFEIARQIYNTLVHAELKKLRQVEQSAEYRQIQVQLREMYQNNTTKTKEFRDIANHRRKLLDAAGFREYDFKSAIKDYYKYFKENIGSNVAIHGIAPLVWTAFENKLYKNGKKVHYKRPGDIYSLRGYSVTGKSGGSEIMFRGTYVEWKGLKLPIKRLPDNDYETAMLEYRVKYVRILRKPGKHKNYWYAQLSLEGKPVIKMNPVSNEPVHPIGSGAVGIDIGPQTIAYASQSEAGLLELASQVQNIERQKGLLQRKMERSRRDMNPENYMPDGTFRRGVKLTKNKSKHYCKLQRELAYLQHRQAETRKRQHIELANHLLSLGDCFYVEDMKWLSLTHRAKETEISEKTGRIKRKKRFGKSIANKAPATLIGILDTKCKSLGIPGVVKVPTIIKASQYNHQSDTYEKKPLGQRWNDMPSGERIQRDLYSAFLLQHLEPDLTGYNKKMLAQDYPQFVEYHNHVINDLSMKPKTLASMGIRRRVS

SEQ ID NO: 242 - protein, 534 aa, unidentified
  REGION 1..534: note = uncultivated organism
  REGION 1..534: note = MG91B-92 Effector
MGWKSKGRGRTRVKRSSTDTCCLTLPLKLEKWQSDRLEKRFEIGRQIYNTVVRAELKKLQQVERTPQYREIEQKLKGAKQEEAKELWKKRTQLLSQSGFSEYGFKSDMKYYYQHFKENIGSSVAVHGIASQVWTAFEKVLFSKDGKMVHFKKKGELSSLRGYSQAGKSGGVEIIFRGSYVEWKGLKLPLKLSPDNDYETEMLSKRVKYVRILRQDGKTKPHWYAQLVLEGKPAIKRDPASGAPKHPIGHGVVGIDIGTRTLAYSADSKVDLIELADRVQNIEQEKMLLQRKLDRSRRATNPGNYNPDGTIKCGVKLTRNKSKRYRKIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 243 - protein, 499 aa, unidentified
  REGION 1..499: note = uncultivated organism
  REGION 1..499: note = MG91B-93 Effector
MSDLSEKKLGNTVLTLPLYPEKWQIDIIEKRFRIMELLKNALIAKELRRLRNLQRTRVWRRLEDEIRSTDKTERKKLYARRTEMLRTAGFSKYGFYEDIELMQKRYIVHIGAQVAQRASSDVWQAFEKMLYGNGDMVHFHKAGSLYSIANKKSGNCMIFRNGCLEWNGGQSRDAVKLILPSKARLTDYERQMLEKPIKYYRVIRKWMKTRWKYYVQITLAGETVHKDRPVGAGRVGIDIGTRTIAVAAEKAVFLRELADGVDENHRRKCALQRKMDNSRRCTNPENYNPDGTVRRPGKGHRPHWVKSRRYLKMQAMVRNLERKNADIRKYQHTCLANEIIRLGTDVYVEQMSYSGLQRRAKETTYDDKGRTKKKKRFGKSLANKAPASFLLILNQKLSYLEPTGLKRVDTKVFRASQLDHTDQSYHSKKLGERWTVLSDGTKVQRDMYSAFLLMNSNAEMNGVDLEQCNQTFEHFKYMHDRAIETIRNDGKRHPASFGV

SEQ ID NO: 244 - protein, 607 aa, unidentified
  REGION 1..607: note = uncultivated organism
  REGION 1..607: note = MG91B-94 Effector
MAKEEFVLTLPLKVEKWQADILDKRYELLRQVYNMVQQKLVRQYIYFSQQKAYRDCKSFEQKREFFENHPFHIKGVKGRNGELCDVKFPYTYRSRGGPSGKRAPNGISDFVSKLGSRKIGEDKTLADFGINSIILEELGLHIMRAWETRLYKPKSSRVSYREYGDINSFGSRKINTNFTGFTINIEDRYIAFNTNGRRGQFADFIQLPIDFSREKNGYAREALNGGIESLKKLLIVRKCIRGGNKYYLQLTISGEKPQKGRTLGKGNVGIDIGPSTIATSSLKGVHFDKLADKCDNIEHELYLINRKLDRSRRANNPQNYNEDGTIKRGIRLVWKKDKDAKRYATLKQQRKELYRKQAEIRKQQHLEMANAMLSQGDTFIVENNPIDAWVRRAKETTKTKTGKNRCKKRYGKSVANHAPSMLVSILKNKVLSLGGNFYEIDPRNAATQFDFTNGEFTKHEVSERSITLSNGCSHQRDMLSAFNLQHIVLQSKELKEYDIEQMEADYPIFCRLESAEQERFKDARHKERDALYYVGASKKQMRRNPQEKSVANRHNGGVGRKHCNTSLGNENAEITGSGLARESIERVSQVPDEYHPFGCDPIVLDGL

SEQ ID NO: 245 - protein, 524 aa, unidentified
  REGION 1..524: note = uncultivated organism
  REGION 1..524: note = MG91B-95 Effector
MKEVFVLELPLRVEKFQEDILNKRYEQLRQLYNYVQGKLLRQYRYFEQMKEFQACTTLKDKRDFIRTHPFHIYGVYGKNGDLFDITFDEYGINGFVAKLGKKLVGTNTTYADLGITSTILDRLSLAIWASWEKMLYSFHTKRLSFKKYGELDSLSNRRIVVRGKVCFPGMELHLEKMELAIKLNSKTGKNAKFITLPILHNPKHADYEMWALKGGIDSIKVITVVRRLVRGQYKYYLQMSIEGEKPQKGRTLGKGNVGIDIGPTTVAVSGENIASIDKLASKCDNIQEEITRLARKIDRSRRANNPENFNEDGTIKRGVRLVWNDSKRYKALRKEMAELQRRQAAIRKQQHIDRANELLKEGDTFIVENNQISGWTRKAKETKVNEKTGKIQKKKRFGKSVANHAPSMFVTILENKVKSLGGEVIKVDTQNAASQYDFTNNSFEKHELKERSVTLSNGDTHQRDMLAAFNLQHLKYDAQEKKQYDTEAMAQHYERFCELEQAEIMRYKNKEKKDDRSTIGAGDL

SEQ ID NO: 246 - protein, 466 aa, unidentified
  REGION 1..466: note = uncultivated organism
  REGION 1..466: note = MG91B-96 Effector
MGTMSVITIPLKTRVYEEDIINKRMELCRKVYNNMLHDRLKALRKMEHDKDYIDSKNTIFTAYKEQDKKVQKEIKKSQIYKDAQEMQKLKLKEYGFSEYDFKSEILDYIRPFNANITAISAGYTIAAPMWSAFDKYLFSNGQMIHYKRYNTVNSIASDGKAGLKIVDSNKKSTRHPESPFGLSLMYGSNRAGKVICMPLIVDKKDLYMQEMICQNIKIVRLVRRKIKGNYKYYVQLTVDCPPAIKFDRDTGEIKNPINKGNVGLFIDTRKITICSNEGVKQIDLIDKKQLEYASVIADISRYLDTSRRISNPENFNEDGTIKKGIVVEGRRTKLQWTNSNNYIKAKNKKADIERRLAEQRKIRAGEICNLILSLGDNIICNKYNFKAAQMRKKETEVNEKGQYLPKSKAGKAISYSAPATLLTMLDNKLVARGYNPINRVDVDKDMQVTDQSRINMAETLFLQLEN

SEQ ID NO: 247 - protein, 509 aa, unidentified
  REGION 1..509: note = uncultivated organism
  REGION 1..509: note = MG91B-97 Effector
MRKKTTDENFIMELPLTVDKFQSDELYKRFELMRRIYNSAQNKLKRQYEYLSSFKAYKECKTKKEKGDFLKAHPFSFRGVLDKDGEPLKITFSEYDLKSYLSKFAKQKVDSQYTYSDFGINTSMLMHMAMAIWSAWKKVLYDPKAQEVSFKKHGELQSIGVDWEPKHPAGLILDLQKGELKVNLNNRQGQYARYITMKIKFGHKEHQIWYEGDALNAGEDSIRGLTIVRRKIRGKYNYFLQMTIRNNKPQKGRTLGTGRVAIDLGTCKIAVVTKNSVRLIPLAPSISSYSDELRIIQRQMDRSRRANNPQKYHEDGTIKKGNHDKWISSKEYIRLRDIKAELERKQAAVRKQDHIALANYLLSLGNEFVIEDNDVQEWAKRKKEEKKREKDGKNLSKAGLGKQIGNHAPAMFVTILKNKVESLGGSFIKVDTDNGATDYDFTNDEFTHHELEERYITLSDGKRHQRDTLAAFNLYHINTVQNRVYDRLAMQRHYAWFCEKEAESVEQYK

SEQ ID NO: 248 - protein, 491 aa, unidentified
  REGION 1..491: note = uncultivated organism
  REGION 1..491: note = MG91B-98 Effector
MQFTVVIPFKPSNELKNDIDKGLKLLTTRYNTEAKKLKKRYDFWKNTKAYKIAEKEGIDSVSKFWEERSFNGEYGIDKLFKINNFKTHTKPKKNIYKNTIVNASMLQILRANLWRSVDKLIKGKGKNIRYVTMQTIPFKSLRGIKWNQKDSVFSFTHKRGHVITQHIDVKTPYELHAFNNCDMRMVTIKKEIIRGKEKYFFHICFEGTPYNKGRALGTASVGIDPSLENMYACFSNGDMEKLSLIQRIDEEFDIYHKVHVNKIDLLRQKLDRQRRANNPQFFNENGTINREALKQANYTWNDSNGYKKTRNALKDIQRKIALRRKQCHFALANYILSKANKIIVEHNKFAAFAKKSQKDTYKPNGQQYSKRRFGRSINHGAPSYFITILKNKALCWGEKASFKLLSEFNGCTKFDHTNQEFNNEIKRNDKVVTLSNGDKVDRDLHAAMNILFCENGIEQNARRRVEKSADHFNIAIMEEFYNQHKDKMIAI

SEQ ID NO: 249 - protein, 483 aa, unidentified
  REGION 1..483: note = uncultivated organism
  REGION 1..483: note = MG91B-99 Effector
MAHKENQKAFVVTFPLRTERWQEDRIDKMMKMLTTYYNDRQKKLVGRYIYLSHSKAFKEAKGKGVVAFKKYMQENGFSKYDLDAFFKANTKGAIYQIGLNSNNIQYLSQCAWSAWERKLFGNGKFIKTDIDVNIFKSRRKNDSFAGFAYDIGSFTITIKSTYARNNICTIPFIVNRKSEYELFAMSQEIRNIAIVRKVIRGVNKYYVQFTVEGAPYNKGRHLGTGTVGIDPGPSKMAIVADKVVEINKLAPNIEEDEKVTARLKRKLDRSRRATNPHMYNEDGTSKKGQRQTIFSKSYDKTRKQLAEAQRKLTAKRKIAHNELANKILEYGDTFKVETNSFKSMQARSKTTSTTKNGRIRSKKRYGKSIKNRAPSEFLLILKNKLLQYSSGKYYDVPASYACTQFDFTDESFTEHKVSERSIITSDGIQHNRDTLAAFNIKNAIVSDTTSKKRKIVKSKEFFDINVMKETYRAFCEMESKLLA

SEQ ID NO: 250 - protein, 311 aa, unidentified
  REGION 1..311: note = uncultivated organism
  REGION 1..311: note = MG91B-100 Effector
MILLAKNNNNTDTCVLTLPLRVEKWQEDCLLKRFEAARRIYNSLLNFELKKLRQLERTVEYRQIQSVISSAYRSNTKDSPECKDAFKRREKLISANGISEYGFKSDVKFFYKHFDGLIGSNVAVHCIAVQLWAAFDKYLRGSGKEIHFKRLGDISSLKGYSVKGKSGGCEIIFRGQYIEWTRKKKKLLLPLKIDPDNEYEREMLSRRVKYVRIIKKPGKNRLRWYAQLTLEGKPALKYSAETGKPIHSVGKGNVGIDIGPRPLHTLPIKKLLCLNLLTESPISSIKKRSFSASWIAAAAQRIRTTTNRTAQ

SEQ ID NO: 251 - protein, 512 aa, unidentified
  REGION 1..512: note = uncultivated organism
  REGION 1..512: note = MG91B-101 Effector
MKKCTTDTCCLTLPLKLEKWQEDRLAKRFEIARQIYNTLVRAEFKKLNELERTPEYKKILEQIHSLDWKDSGSRQRLNDLYRERNQILKNAKFTEYGFKSDMKFYYKHFSDNIGSCVAVHGIAPQVWAAFEKYLFKPEGKKVHFKRFGDVHSLRGYSKAGESGGAEITFCGTHIRWKNLELPIKLSPDNAYETEMLSRRVKYVRILRKPGKSKDRWYVQLSLEGKPAIKYDRNAGEIKHPVAHGAVGLDIGPQTLAYSAETTADLVELADQVQNIEQEKRRLQRKMDRSRRSTNPGNYNTDGTIRRGVKLTRNKSKAYCKLQRQLAYLQHTQAETRKRQHTELANYLLSLGDCFYVEEMKWSSLAHRAKETAVSEKTGRFKQKKRFGKSIANKAPSTLIGILQQKCESLGLPGIIYVPLSAKASQYNHLTGEYTPKPLSQRWNTMPDAQRVQRDLYSAFLLQHFDSQAGTYNLQSLQRDYPQFLQYHDQSIRRLSEMPKTLSSMGIRKSIRA

SEQ ID NO: 252 - protein, 612 aa, unidentified
  REGION 1..612: note = uncultivated organism
  REGION 1..612: note = MG91B-102 Effector
MTENFVLTLPLKVEKWQADILDRRYELLRQTYNMVQRKLVRQYIYFSQQKAYKACKGDFSQLREFFASHPFHFKGITSKNGELSDIKFPYTYESRSSGSGKRAPNSISDYVSKLWTHRIGQDKTLADLGINGSILEELGLHIMSAWGKRLYDPKASRISFKDYGDVNSFGSRYHKGNFAGFTINLEEGFITYNANGRRGRYADIIKLPIDFSREKNDYAAYALKGGIESFRKLLIVRRCIRGRNKYYLQLNINGEKPQKGRTLGKGNVGIDIGPSTIATSSLQGVHFDKLADKCDNLEHELYLINRKLDRSRRANNPHNYNEDGTIKRGVRLMWKQDKDVKRYASLKQKRRELYRRQAEIRKLQHLEMANAMLSQGDTFIVENNPIDAWVRRAKETTKTKTGRNRCKKRYGKSVANHAPAMFVTILKNKVQSLGGQFLEVDVRYAATQFDFTNGIKTKHEVSERSITLSNGNTHQRDMLSAFNLQHIRQESIGTEDKDYDIEQMQADYPIFCKLETAEQERFKVARHKERDALYFLGASKKQLRRQPQDESVANRHNGGVGRKHCNTRLGSNNQENAKIRGSGHVRESIERVSQVPDGYHLVGNHPIILDGL

SEQ ID NO: 253 - protein, 525 aa, unidentified
  REGION 1..525: note = uncultivated organism
  REGION 1..525: note = MG91B-103 Effector
MKEVFVLELPLQVEKYQEDILNKRYEQLRLLYNYVQRKLLRQYRYFEQMDDFQACKTFKEKRDFFKSHPFHINGIIGSNKELLDITFDDYGINGFVTKLGSKSISDENTYSDMGINSAILAKLSDNIWSSWDKMLYDHKAKRISFKRYGDLNSFGCGKKTNGSGKKVFSGLELHLETMTLAIKLNSKVGKSAKFITLPILHNPKHADYEMMALKGGIDSVKVVKVVRRMVRGRYKYYLQMTIEGEKPKKGRLLGKGNVGIDLGPTTVAVSGESIASIDKLASKCDNIQEEITRLARKIDRSRRANNPENYNEDGTIKCGVHLAWNNSKRYKALRREMAELRRKQAAIRKHQHIDRANALLKEGDTFIVENNQISGWTRKAKETKVNEKTGRIQKKKRFGKSVANHAPAMFVTILENKVKSLGGEVVKVDTQNAASQYDFTNDSFEKHELKERNVTLSNGDTHQRDMLAAFNLQHLKYDEAEKKQYDREAMAQHYERFCELEQEEIMRYKNKVKKDDRSTIGTGNL

SEQ ID NO: 254 - protein, 521 aa, unidentified
  REGION 1..521: note = uncultivated organism
  REGION 1..521: note = MG91B-104 Effector
MNEVFVLELPLQVEKYQADILNKRYEQLRQLYNYVQGKLLRQYKYFEQMKEYQACKAFKEKKDFFRSHPFQINETLKGNETALKVSFDEYSIKGYIEKLVQKSVGQNMNYADLGFTTNILDHLGASIWAAWDKKLYDFKAKRISFKKYGELDSFSSRRKVVGGKVYFSGMELYLDTMELAIKMNGKVGKEAKFITLKMLHNPKHADYEMWALKGGVESVKVVKVVRRLVRSQYKYYVQLTIEGEKPQKGRTLGKGNVGIDLGPTTVAVSGENIVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISGWTTRAKETKVSEKTGKIQKKKRFGKSVANHAPSMFVSILENKVNSLGGQVVKVDTQNAASQYDFTDGSFTKHELKERNVTLSNGDVHQRDMLAAFNLQHLTGEKRQYDVEAMKRDYERFCQLEQEEIQRYKNKEKKDDRGTIGAEKL

SEQ ID NO: 255 - protein, 509 aa, unidentified
  REGION 1..509: note = uncultivated organism
  REGION 1..509: note = MG91B-105 Effector
MARCTTPTCVLTLPLRLEKWQEDRLAKRFEIARKIYNTFLGVKLKELRRLEQTPEYRAIKKELAALYSEKEKNADRIRALYEERNALLKNFTEFKFKKDIKFYYKHFNSNIASKVATHCIASQVWSAFEKYLFGNGKRIHFKRPGEIYSLRGFSATNARSGGSEIIYRGDHIEWNKLRLPLKLDPGNQYEEEMLTNRVKYVRILRRPGKNRDHWYAQLTLEGLPSIKRDDETGDPVHPIGNGPVGIDIGPQTIAYSSAKEAGLLELADRVQNIEDKKCRIQRKMDRSRRATNPGNYNPDGTIKRGVPLTRNKSKRYLRLQKELAWIQHQQAAIRKEQHVALANYLLTLGDRFYIEDMNWQGLARRAKGTAVPEEEKPHKRKKRFGKSIANKAPAALVTILKNKCTSLGLPGVIPINTKVRASQYNHLTDSYQKKELSQRWNEMPDGRKIQRDLYSAFLLQHMNSQLDGFDRAALQRDYESFVSLHDQAILRAAAAPKLISSMGVKRTAN

SEQ ID NO: 256 - protein, 551 aa, unidentified
  REGION 1..551: note = uncultivated organism
  REGION 1..551: note = MG91B-106 Effector
MSNLLFLLENEEKMRYNGVEKQREGEGTRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSTDSEVNLLELADRVQNIEQEKRRLQRKMDRSRRAMNPGNYKPDGSIKQGVKLTWNNSKRYKRIQHQYAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCRRDLPEYNRKELQKEFGEELYRVFDREALQRDYPQFVAYHHMTIQRLSELPHTIPSMGIRRIIS

SEQ ID NO: 257 - protein, 615 aa, unidentified
  REGION 1..615: note = uncultivated organism
  REGION 1..615: note = MG91B-107 Effector
MAKENFVLTLPLRVNKCQADILDKRYELLRQMYNMVQQKLVRQYIYFSQQKAYKACENFEQKREFFNAHPFHFKGIKGKNGELCDIKFPYTYESRSSSSGKRAPSGISDYVSKLGSHKIGQDKTLADFGINSYIMEELGLHIMKAWEDRLYKFKSKRVSFKDYGEINSFGSRYKNGNFAGFTINLKEGFITYNTNGRRGQYADLITLPIDFSREKRGYAAEALKGGVDSLRKLVIVRRCIRGRNKYYLQLTISGEKPQKGRTLGKGNVGIDIGPSTIATSSLYGVHFDELADKCDNIEHELYLLNRKLDRSRRANNPDNYNPDGTIKRGVRLVWKKDKDVKRYASLKQLRKELFRKQAETRKQQHLEMANAMLSQGDTFIVENNPIDAWVRRAKETTKTKTGKNRSKKRYGKSVANHAPAMFVTILENKVKSLGGQFHRIDPGYAATQFDFTNGVFTKHEVSKRSITLSNGRTHQRDMLSAFNLQHARLEAEKAKSRDKEKKVESYDIVQMQADYPIFCRLETAEQERFKDARRNERDALYFVGAFAKKLRHEPQEETVANRHNGGVGRKHCNTLCTENAKITGSGLVRESIERVSEAPEECYHAGDQSIILDGL

SEQ ID NO: 258 - protein, 528 aa, unidentified
  REGION 1..528: note = uncultivated organism
  REGION 1..528: note = MG91B-108 Effector
MKEVFVLELPLRVEKYQADILNKRYELLRQLYNYVQGKLLRQYRYFEQMKDYQICKTFKDKREFIKAHSFHINGVLGRNKELLDISFDEYGIMGLVTKLGQKLIGSGKTYTDMGISSTILANLSDNIWSSWNKKLYDYHADRITFKKYGSLNTFGSGKIVQKKDNEVKEYFPGMVLHLERMEIEIKTNGGLGKKATFITLPILHNPKHADYEMMALAGGSSSIKVLTIVRRLVRGQYKYYLQMSIEGEKPQKGRMLGKGNVGIDLGPTTVAVSGENIACIDKLASKCDNIQAEITKLARKIDRSRRANNPQNYNEDGTIKRGIKLVWYDSKRYKELRRELAELRRKQAAIRKQQHIDRANALLKEGDTFIVENNQISGWTTRAKETKVNEKTGKIQKKKRFGKSVANHAPSMFVTILENKVKSLGGQVVKVDTQNAASQYDFTNDSFTKHELKERNVTLSNGDTHQRDMLAAFNLQHLKYDEKEKKQYDKEAMKAHYEHFCELEQEEIQRYKNKEKKDDRSTIGAEKL

SEQ ID NO: 259 - protein, 609 aa, synthetic construct
  REGION 1..609: note = uncultivated organism
  REGION 1..609: note = MG91B-109 Effector
MAKETFVLSLPLIVEKWQADILDKRYELLRQTYNMVQQKLLRQYIYFAQQKTFKACENFQQKRDFFASHPFHFKGIKGRNGELSDIKFPYTYESRSSSSGKRPSGGISDYVSKLWAHRISQDKTLADFGINGSILEELGLHIMKAWETRLYEPKASRVSFKDYGTVNSFGCRYHKGNFAGFTINLEEGYITYNTNGRRGSLANIIKLPIDYSREKKGYASEALKGGVDSLRKLVIVRKIIRGRNKYYLQLTISGEKPQKGRILGHGNVGIDIGPSTIATSSLQGVHFDKLADKCDNIEHELYLVNRKLDRSRRANNPQNYNEDGTIKRGIRLVWKKDKDAKRYASLKLLRKELYRRQAEIRKLQHLEMANAMLSQGDTFIVENNPIDAWVRRTKETAKTKTGRNRCKKRYGKSVANHAPAMFVSILKNKVLSLGGQFHEVDVRYAATQFDFTNGVKTKHEVGERSITLSNGRTHQRDMLSAFNLQHITLESIGTEDKDYNIEQMYADYPIFCKLESAEQERFKVARKNERDALYFVGVSEKNLRHQHHKETVVNRHNGDVGRKHCNNSLEKENAKITGNGLIRESIERVSEVPDKYHLVGNHPIVLDGL

SEQ ID NO: 260 - protein, 326 aa, unidentified
  REGION 1..326: note = uncultivated organism
  REGION 1..326: note = MG91B-110 Effector
MNEVFVLELPLMVEKYQADILNKRYEQLRQLYNYVQGKLLRQYRYFEQMADYQVCKTIREKRDFLKNHPFNINGVLDRRGKLLDITFDEYGIQGFVEKMIHKSVGNNISYADLGLNTLILDYLAASIWRAWDKKLYDLNAKRISFKKHGELDSFSSRRKVVNGTARFFGLELYLDKMELAVKVNGKQGKNAKFITLKIQHNPKHVDYEMWALKGGVDSVKVVKVVRRLVRSQYKYYVQLTIEGEKPQKGRTLGKGNVGIDLGPTTVAVSGENIVCIDKLASKCDNIQDEITRLSRKIDRSRRANNPQNFNENATIKRGIRLVWNDS

SEQ ID NO: 261 - protein, 502 aa, unidentified
  REGION 1..502: note = uncultivated organism
  REGION 1..502: note = MG91B-111 Effector
MAQEFTLVIPFKPSNELKHDIEQGFKLLTIRYNTELKKLTKRYDYLIKTPEYEIAKREKHEKTFWINKGFTKNGFHKFFRINYFKKTEKSSSSIYDNTIVSSKMLEALKDNLWASFEKLLDGQGKDVHIKNISSMTFKGLCQIKYNPQTQSFDFTHRGRRNIISQKLDIKTDYELHAFANTMRQVTIKKQIIRGKEKYFFHICFQGTPYNKGRELGTNSVGIDPSMLKMFACYSNGEMIEHSLLDKIDEKSGEICDSKYNSDNYEKLEKQVGALQTQINKQRRIKNPQFFDENGKINEEALNKAKLEHPNSSPWEDSKTTIRRKNKLAQIKRNLALHRKECHFRLANYILSKANTINVENNSYKSFQARAKETTYNTNGRPRSKKRFGKSIQKGAPSAFITILKNKAASWGDRVSFSEVGASEACTQFDHTSSEFTKHELKERVVELSNGDRVHRDFHAAMNILFFKNTVTKSKKGTKKDSDHFDIEGMREFYNQHRENMIT

SEQ ID NO: 262 - protein, 534 aa, unidentified
  REGION 1..534: note = uncultivated organism
  REGION 1..534: note = MG91B-112 Effector
MGWKSKGRGGTLVKKSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQETFGNELYRVFDREALQRDYPKFVEYHRETIQRLSALPKTIASMGIRRIIS

SEQ ID NO: 263 - protein, 578 aa, unidentified
  REGION 1..578: note = uncultivated organism
  REGION 1..578: note = MG91B-113 Effector
MAFGSKNSTPSFVLTLPLDINLNEQDYLYKEFKKCGIIYNQLVSVTIKMWHQLRKTRKYRELMAAIAKSASDSDEQKTLYKQREKILKEHHFSEDSFHKLVVPYAKHYAIHSHVAQAIATDVWKAWKSFFFSKGKEVHYKRLDDVSSISGKNNATGIVLRPAKLTTSNLASAKRKVQKTVEQRYYKAYGQSDPKDDKKVILPDSVIPQMEAEIAVAVAKVKPAVGNGSLRIVYGKHEFPVVVRNSNTQTGKYQQEALKCGVKFCRITRKWVKIKWKYYAQLVLEGYPPTKCDSNGVIKHPIKQGCVGLDIGTQTLAISGSDICDLRLLAPSARAQAKGLVNEIARTQRAMDRSRRATNPDCYNPDGTIKRLKCQHGHKQKRDWKYSKRYYRLRARLRDLNRRLADIRKMEHNILANELLEHGNEFIVEDMNYKALQKRSKETKVNPKTGRTHTKKRFGKSLSRCAPAIFISILGKKASRYGSSVIKVSTFETKASQFDHTDESYTKKKLSERMAHLSSGEVVQRDLYSAFLLEHIDTESLEYNTEALKSAFPAFLKMHENTKKHLQKEENNLPASVGF

SEQ ID NO: 264 - protein, 534 aa, unidentified
  REGION 1..534: note = uncultivated organism
  REGION 1..534: note = MG91B-114 Effector
MGWKSKGRGGKRVKRSSTDTCCLTLPLKLEKWQSDRLEKRFEIGRQIYNTIVHAELKKLRKLERTQQYQEIEQKLENAKQEETKELWKKKRQLLDQNGLSENHFKADMKYYYPHFKDNIASSVAVHGIASQAWTAFEKVLLSKDGKMVHFKKKGELSSLRGYSQAGKSGGIEIIFRGSYVEWKGLKLPLQLSPNNEYETEMLSKRVKYVRILRKDGKTKTHWYAQLALEGKPAVKRDSISGAPKHPVGHGVVGIDIGTRTLAYSADSKVDLIELADRVQNIEQEKMLLQRKLDRSRRATNPENYKPDGSIKQGVKLTWNNSKRYKRIQHQYAMIQHHQADIRKQQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCRRDLPEYNRKKLQEAFGNELYRVFDREALQRDYPKFVEYHRKTIQRLSALPKTIASMGIRRIIS

SEQ ID NO: 265 - protein, 567 aa, unidentified
  REGION 1..567: note = uncultivated organism
  REGION 1..567: note = MG91B-115 Effector
MSNLLFLLENVRKMRYNGVEKHWKGEGTRVNNEEKEKEGATCCLTLPLKLEKWQSDHLEKRFEIARQLYNTLVHAELKKKCQVERMPEYQEIMKMIDTASNQKEKWALEKKRKAFLEENGFSRNAFKADMEKYYKHFNDNIGANVALHGITAQVWIAFDKVFVDNYKQKGKVHFKKPGELCSVQGYSQKKSGGKEIMFREDRIIWNQQGNRDRKKVKREFLLKLSPNNPYEAEMLKRHVKFVRILRKDGKTKPHWYAQLVLEGKPAVKRDPISSAPKHPVGHGVVGIDIGTRTLAYSTDKEVNLLELADRVQNIEQEKRRLQRKMDRSRRAMNPENYNPDGTFKRGVKLTRNKSKRYRRIQHQLAMIQHHQADIRKQQHNELANYLLTLGDCFFVENMSYCDLVHRANKTEISEKTGRYKRKKRFGKSIANKAPAMLITMLKQKCQSRGLKGVKEVDTHVRASQYNHQTDTCIEKALENRWNVMPDGERIQRDLYSAFLLQHCKQGLPEYNRKELKKEFGEELYRVFDREALQRDYPQFVAYHHMTIQRLSELPHTIPSMGIRRIIS

SEQ ID NO: 266 - protein, 524 aa, unidentified
  REGION 1..524: note = uncultivated organism
  REGION 1..524: note = MG91B-116 Effector
MTKERTLFMKKMSVLELPLDVQPWQAERLNKRYKIAAMVYNAFRDYEWNKIICIRETKQWREELSDSILRDLYDELKNAKEKESASEEKRINKQIKDIYDRRGKLYKDNGITRFSFLEDIKRFCSPYSTIIPSNMAQLTIAAPLYTAVERCLYGNGDFPRYKRIINSNILKTNGKSGIRLMNDDRGYFLLVSNRRQGAKPMTLPVKVSDSNYNMDMINRDIKIVAIVRKRINGKYKYYTQLTVEGAPFQKVNKDGEYIHQIGNGKVGLNIYKNKVSVASDSGVFEFDLTPDMDAYQSELNELTRCQEAIRRRLNPQNYNDNGTVKKGIYNPETRKTERLHWNSSIKYDEISNKRRELQRVYIERRTIHVNNMIHKILACGDQIIVPDYCFNKDKRKNTKGDEVPINELKKRKDRRIEIQQGAPASFLARLNKVLEQYDREPVSRIRLSEDNFFYCHMSDVDNKDFFKRDHIELPNGTRCSQGLYKAFVLLHYNSDTGEFNHDSICEDFNKFLNMVEAYQSKSTQ

SEQ ID NO: 267 - protein, 510 aa, unidentified
  REGION 1..510: note = uncultivated organism
  REGION 1..510: note = MG91B-117 Effector
MAKATTDTCCLTLPLVLEKWQSDRLAKRFELARQIYNTLLNYELKKLRRLEKTEEYAAGQAKLKELNDRQERDEAARKECHRQLNELRKAAGFSEYDFKTDVKPFYKHFGDNVGSSVAVHGIASQVWQAFERLFYGNGKAVHFKKRGEIRSLRGYSVSGKSGGVEIMFRGSYIEWKGLRLPIKLDADNAYEAEMLQKRVKYCRIVRKEGRRKTRWYVQLMLEGKPALKCDPDTGALRHPTGSGPVGIDIGPQTVAYAAETEAVLQELADGVKDIEREKRTVQRKLDRSRRATNPECFAADGTIKRGVKLNWRKSKRYLALQRELASLQRQQAEIRKRQHTELANHLLSLGDRFYVEDMAWPALTHRAKETAISEKTGRYKRKKRFGKSVGNKAPAMLIGILDQKLRSRGMGGVVKVPTTVRASQFNHVTGEYQKKPLSQRWNEMPDGRRIQRDLYSAFLLQHVNEAQDGFDVAALIRDYENFVTLHDRMIETLKTAPKTLTSMGIWRTAS

SEQ ID NO: 268 - protein, 566 aa, unidentified
  REGION 1..566: note = uncultivated organism
  REGION 1..566: note = MG91B-118 Effector
MGWKSKGRGGTLVDNEEKKEDGATCCLTLPLKLEKWQSDHLEKRFEIARQLYNTLVHAELKRIHRIERMPEYREIERLIEATTDKKEKWALKNQKKQFLRKNGINRNTFTTDMKRLYKHFNDNIGSNVAQHGIVSQVLTAFEKVYIENQSGEVHFKKPGDLRSVQGYSQKESGGKEIMFRNGKIIWTQQENDDRKKSPKYQQRKKRQNYKGVKGIAREFPVVFSKNNPYEAEMLTKHVKFVRILRKDGKTKPHWYAQLVLEGKPAIKRDPASGAPKHPVGHGVVGIDIGTRTLAYSADSKVDLIELADRVQNIEQEKLLLQRKLDRSRRATNPENYKPDGTIKKGVKLTRNKSKRYRKIQHQLAMLQHRQADIRKRQHNELANYLLTLGDCFFVEDMDYRALTRKAKKTEISEKTGRYKRKKRFGKSIANKAPSMLIALLDQKCKSLNLEGVKKVDTAVKASQYNHQTDECKAKELKERWNIMPDGERIQRDLYSAFLLQHCKRGLPEYNRKKLQETFGNELYRVFDREALQRDYPKFVEYHRKTIQRLSALPKTIASMGIRRIIS

SEQ ID NO: 269 - protein, 521 aa, unidentified
  REGION 1..521: note = uncultivated organism
  REGION 1..521: note = MG91B-119 Effector
MAKSKQNKDENKNFVLTLPIKMNVCDEHILDKRYEYLRQIYNIAQRRLLNQFMYFAVTKEYKSIKTIKEKKDFFKTHPFKIKGYDKKITFTEFSIKSYLRNFAKLHVSKDKTYKDFGINTKILDGIFNNIWRAWNKRLYELKVKKIHFKEHDELNNITFYKSNNYFVGLNVDLSNMVIKMNVNGCRMSKAKFITFNLNKKLNDYETFALKGGCDSIKVISIVRKEIRGKKKYYCQFCIEGEKPMKNRILGKGQVGIDLGTSTIAVSSLNGVSIDILAEKCNKIDRETYIIDRKMDRSRRATNPQNYTEDGQIVRNKKGETRKWNYSKHYIKLRSKRKELYRKNAAIRKIEHEMTANKLLSLGNVFVVENNPIKGWQTRAKETTINKKGKYNSKKRYGKSIANHAPSEFITILENKVVSLGGKFEKVDIKNSASRFDFTNETFNEHNVGVRKITLSNGNIHQRDMLASFNLQHLVIPNENLKEYDVEQMKLDYPIYCKLEKQEIALHKNGNKKQYKSVIGKW

SEQ ID NO: 270 - protein, 511 aa, unidentified
  REGION 1..511: note = uncultivated organism
  REGION 1..511: note = MG91B-120 Effector
MKRSGTDTCCLTLPLRLEKWQEDRLAKRFEIARQIYNTMVRAELKKLRKMELSQPYRSIQEQIKALDWKNQNDKQQLRSLYNERDQLLKKNGFTEYGFKSDIKYYYKYFNDNIGSSVAVHGIAPQVWAAFEKMLFRKEGKKVHYKKKDDIHSLRGYSVTGKSGGVEIIFHETYIEWKGLKLPLKLSPHNAYETEMLSRRVKYVRLLRKPGKRKDRWYAQLSLEGKPAIKHNPASGELRHPVGQGAVGLDIGPQTLAYSACCAADLVELADRVQNIEQVKRLLLRKMDRSRRATNPDNYTDDGTIRRGVKLTHNKSKRYQRTQRELRYLQHCQAEMRKRQHIELANHLLTLGDRFYVEDMAWPSLTRRAKETAISEKTGKIKRKKRFGKSIANKAPAALIGILEQKCYSLGLPGVVKVPTSLKASQYNHQSGEYTPKPLSRRWNNMPDGERIQRDLYSAFLLQHYDPQTGNFDPVLLQQDYPQFVQYHHCAIQRLSAMPRTLSSMGIQRSLR

SEQ ID NO: 271 - protein, 511 aa, unidentified
  REGION 1..511: note = uncultivated organism
  REGION 1..511: note = MG91B-121 Effector
MARCTTPTCVLTLPSHLEKWQEDRLAKRFEIAQKIYNALLGAELKKLRRLEQTPKYRAIKKDLAALYGDKEKNADRIRALYKKRNKLLKDNSFTEYGFTVDAKLYYKPFRTNIGSCVAVHCIAPQVWSAFEKYLFGNGKRIHFKRPGEIYSLRGYSSKTSSGVEIIYRGDHIEWNKLRLPLKLDLGNQYEEEMLTNRVKYVRILRRPGKNRDHWYAQLTLEGLPAIKRDDETGDPVHSIGNGPVGIDIGPQTIAYSSSKEAGLLELADRVQNIENKKCRIQRKMDRSRRAANPGNYNPDGTIKRGVPLTRNKSKRYLRLQNELAWIQHQQAAIRKEQHVALANYLLTLGDCFYIEDMNWQGLARRAKETAIPEPEKERKCRRKKRFGKSIANKAPATLVTILKNKCTSLGLPGVIPISTKVRASQYNHLTDSYQKKELSQRWNEMPDGRKIQRDLYSAFLLQHMNSQLDGFDRASLLRDYESFVSLHDQAILRAAAVPKLISSMGIKRTAN

SEQ ID NO: 272 - protein, 528 aa, unidentified
  REGION 1..528: note = uncultivated organism
  REGION 1..528: note = MG91B-122 Effector
MNEVFVLELPLQVENYQADILNKRYEQLRQLYNYVQGKLLRQYRYFEQTKEFQECKTFKAKREFIKAHPFQIKGVLGRNKKLLDIFFDEYGILGLVAKLGQKPIGSRKTYTDMGISSTILANLSDNIWSSWNKKLYDYHANRVSFKKFGELNTFGSGKIIQKKDNEVKEYFPGMVLHLDKMEIDIKTNGALGKKATFITLPILHNPKHADYEMMALAGGSRSIKVLNVVRRLVRGQYKYYLQMTIEGEKPQKGRTLGNGNVGIDLGPTTVAVSGENVVSIDKLASKCDNIQEEITRLSRKIDRSRRANNPQNFNEDGTIKRGIKLVWNDSKRYKELRRELAELRRKQAAIRKQQHIDRANALLKEGDTFIVENNQISGWTTRAKETKVNEKTGKIQKKKRFGKSVANHAPSMFVSILENKVKSLGGEVVKVDTKNAASQYDFTNGSFTKHELKERNVTLSNGDTHQRDMLAAFNLQHLKYDEKDTKQYDTEAMTADYERFCQLEQEEIQRYKNKEKKDDRSTIGAEKL

SEQ ID NO: 273 - protein, 525 aa, unidentified
  REGION 1..525: note = uncultivated organism
  REGION 1..525: note = MG91B-123 Effector
MNEVFVLELPLQVEKYQADLLNKRYEQLRQLYNYVQGKLLRQYRYFEQMKEFQACKTFKEKKDFFRRHPFHIDGILGSNKELYDITFDEYGINGFVGKLGKKQVSAGVTYADLGITSTLLDRLSLAVWAAWDKMLYDYHAQRVSFRKYGELNIFGSRKKTNGSGKKVFSGMELHLEKMEVAIKLNGKVGKAAKFITLPILHNPKHIDYEMWALKGGFDSVTVLTIVRRLVRSQYKYYVQLTIEGEKPQKGRKLGKGNVGIDLGPTTVAVSGVNVVSIDKLASKCDDIQEEITRLSRRIDRSRRANNPQNFNEDGTIKRGIKLVWNDSNRYKELRRELAELRRKQAAIRKQQHIERANALLKEGDTFIVENNPISSWTTRAKETKVNEKTGKIQKKKRFGKSVANHAPSMFVSILENKVKSLGGDLVKVDTKNAASQYDFTNNSFTKHELKERNVTLSNGDTHQRDMLAAFNLQHLQYDEKDDKQYDTEAMTADYERFCQLEQEEILRYKNKVKKDDRSTIGAEKL

SEQ ID NO: 274 - protein, 511 aa, unidentified
  REGION 1..511: note = uncultivated organism
  REGION 1..511: note = MG91B-124 Effector
MEKEFFVIELPLKIEKWEADLINKRYELLRSIYNYAQNKLLRQFKYFEQMTEWKNCKTKKNKSDFFKKHPFYFKGISSKITFSEYDITAFVEKLIKKQVSSNKTYKDFGINSSIAESIGKHIWSAWEKKIYNKGEKISFKKKGKLNSISVRKKSGYFGGIDVDLENMLLRLNINGKIGKKSNKITLPIVFDKPTEYEMYSFKNGIDSINVVTIVRKLIRGNYKYYVQFTIEGKKPQKGRVLGKGKVGVDVGPSTVAVSSINGVQIITLAEKCDGIEHKCKLLERKMDRSRRATNPQNYNDNGTIKKIKNEHLKWVYSKRYSKIRNKKCELQRKQAAIRKLQHNINTNELLKFGDSFIVENNPISAWQHKAKETKINKNGKIQSKKRFGKSIANHAPAMFITILENKVKSLGGELIKVDIKNAASRFDFTNSDFVEHKLNERKITLSNGNTHQRDLLAAFNLQHLNSDDESLKSYNIEEMKKDYPIFCKLEETEIDKFVNGQKKSHKYTVGY

SEQ ID NO: 275 - protein, 524 aa, unidentified
  REGION 1..524: note = uncultivated organism
  REGION 1..524: note = MG91B-125 Effector
MKEVFVLDLPLRVEKYQSDILNKRFEQLRQLYNYVQGKLLRQYRYFEQMKEYQACKTFKEKKDFFRNHPFHINGILGSNKELLDITFDEYGIKGFVEKLVQKGVGTDMTYADLGFTTYILDHLSAGIWSAWDKMLYDYKARRISFKKYGELDSFSSRRKVVQGKVYFSGMELHLDSMMLSVKMNGKVGKAAKFITLPILHNPKHADYEMWALKGGLDSIKVVKVVRRMVRGRYKYYLQMSIEGEKPLKGRKLGKGNVGIDLGPTTVAVSGENIASIDKLASKCDSIETEKAKLGRKIDRSRRANNPQNFNEDGTIKRGIRLEWNDSKRYKELRVEMAELQRRQAAIRKQQHIDRANELLKEGDTFIVENNQISGWTRKAKETKVNENTGKIRKKKRFGKSVANHAPAMFMTILENKVKSLGGKVVKVDTQNAASQYDFTNNSFTKHELNERSVTLSNGDTHQRDMLAAFNLQHLKYDEQEKKQYDTEAMTKHYGRFCELEQAEILRYKNKEKVDDRSTIGAKEL

SEQ ID NO: 276 - protein, 514 aa, unidentified
  REGION 1..514: note = uncultivated organism
  REGION 1..514: note = MG91B-126 Effector
MIVEKYQADILNKRFEQLRQLYNYAQGKLLRQYRYFEQMKEFQACTTFREKKDFLRSHPFHVNGILDRSGDVLNITFDEYGIKGFLEKLIHKPVGSNMTYADLGITTYILDHLSAGIWRAWDKMLYDYHAKRVSFKKYGELDSFSSRRKTSGNKVYFAGMELHLDTMELSVKLNGKQGKDAKFVTMPILHNPKHADYEMWALKGGEDSVKVVKVVRRLVRGQYKYYVQLSIEGEKPQKGRTLGKGTVGIDLGPTTVAVSGENVVSIDKLAAKCDNIQSEITRLARKIDRSRRANNPQNFNEDGTIKRGVRLVWNDSKRYKALRREMAELQRRQAAIRKQQHIDRANELLKEGDTFIVENNQISAWTRKAKETKVNEKTGKIQKKKRFGKSIANHAPSMFVTILENKVNSLGGKVVKVDTQNAASQFDFTDGSFEKHELKERSVTLSNGDTHQRDMLAAFNLQHLKYDDAKKKQYDTDAMGQHYERFCELEREEIERYKKKEKKDDRGTIGAKGL

SEQ ID NO: 277 - protein, 533 aa, unidentified
  REGION 1..533: note = uncultivated organism
  REGION 1..533: note = MG91B-127 Effector
MNEVFVLELPLRVEKYQADILNKRYEQLRQLYNYVQGKLLRQYRYFEQMREFQDCKTFKAKREFIKAHPFQISGISGSNKQPLDITFDEYGIRGFVEKLAQKSAGPNVTYADLGFTTYILDHLSVSIWSAWDKMLYDYHAKRVSFKKYGELDSFSSRRKIVGGKVYFSGMELHLDTMELSVKLNGKQGANAKFITLPILHNPKHADYEMWALKGGLVSVKVVKVIRRMVRSQYKYYVQLTIEGEKPQKGRTLGKGNVGIDLGPTTVAVSGDNIVSIDKLASKCDNIQKEITRLARKIDRSRRANNPQNFNEDGTVKRGIKLVWNDSKRYKELRREMAELQRRQAAIRKQQHIDRANELLKEGDTFIVENNQISGWTRKAKETKVNEKTGKIQKKKRFGKSVANHAPAMFVNILENKVKSLGGEVVKVDTQNAASQYDFTNDSFEKHELKERSITLSNGDTHQRDMLAAFNLQHLKYDDKEKKQYDTEAMKEHYERFCELEQEEIQRYKDKEKKDDRSTIGAEKLHMNDGTVKK

SEQ ID NO: 278 - protein, 497 aa, unidentified
  REGION 1..497: note = uncultivated organism
  REGION 1..497: note = MG91B-128 Effector
MAKNEQNCCVLTLPLLTEPWQEHIIETRFDIVEKLKNTLIGFELRKLKQLERTRAWKRLMKSIRECSDEKQKKSLFKERNTMLKDAGFTEFGFSGDVTKLAVHKHFVPHIGANIAQRSGQDVWRSFEKYLFSNGKAVHFHKRGSLDSIACKKPRGDGMTIDSDVFTWKGGRCDDCVILKIRIRRPKNAYEREMIQKEIRNMRVVRKWIKTRYKYYLQITYQGAPVSKHRPVGKNRVGIDIGTQTIAWSSPTEVRLAELAAGIRRNHAEKTALQRKMDRSRRTANPQNYNPDGTIKRGIKLRWVKTKHYLRMEGRVRELERKNADIRKNEHICLANHILSLGKEVYIEDMNFKGLQGRAKETKVDKNGRFARKKRFGKSLANRAPSMFVTILENKLKSAGGNLYKVNTRTFKASQYDHTADEYRKKTLGQRWNHLKNGDKIQRDLYSAFLLMNAAPTLEEPDKKLCKKTYPQFALLHDAEMERLNAEEKPHLSSMGIR

SEQ ID NO: 279 - protein, 523 aa, unidentified
  REGION 1..523: note = uncultivated organism
  REGION 1..523: note = MG91B-129 Effector
MEKELFILELPLKVEKWQEDLLNKRYEYLRQIYNYAQGKLLKEYNYLSQMADFKNCKTKKAKSEFFNTHPFIIKGFKKPVIFSKMKSKEIEADVYGITDFIEKIVQKKVDNKKTYKDFGINTTMINALANHIWAAWEKVIYGNGKHISFKKYGDINTISIRKKGNFNGLDVDINNMFLAFNINGRKRSNSKKIKLLIDYKKLTDYEIYSLKNGYDSIRVVTIARKLIRGKYKYYVQFTIEGEKPQKGRTLGKGNVGIDVGPSTIAVSSLKGVSIDTLSEKCNDIEHDLYIINRKIDRSRRATNPQNFKEDGQIKPINRREGQKRHWNKSKHYVNLSNKKRELLRKQAAIRKLQHNIKANELLTLGDTFIVENNPISAWVKKSKETKINKKGKFQSKKRFGKSVANHAPAMLITILENKIKSLGGKFIKVDIKNAASRYDFTNNEFKEHGLNERKITLSNGNTHQRDLLAAFNLQHIDLTTEEFKKYDVNEMVKDYPIFCNLEKEEIQRFLDGLKKRHNSTIGI

SEQ ID NO: 280 - protein, 449 aa, unidentified
  REGION 1..449: note = uncultivated organism
  REGION 1..449: note = MG91B-130 Effector
MSDVKGRYILTLPLKTEVYQEHILEKRFRLGEHLYNNFLNFEKKKFYEMTKRKDYRDAQNLIKELTIQINSLDEFKESKQLIQELRKQRKEQYDIIYNIKKMFDWSEFGFTNDMKKYRKYYKANLNSTICDNLSGRCFKAFEKMESELIKDQYRNDEDKVNPCVHYKRKDSLRCLTAKKNSTGIRFSLDEGNRIGTVKWQGLVFSIDLSKCTLYEWQALQSDICYCAVKREKIKGKWKYYIQITLKGHVPDKFDKQTGELKRQLGKGNVGLYFTSTSLTVSTEDGVKTYPLEIKNHEDKKSDLLQKMDASRRATNPDNYNEDGTSKEREEIKGWHFSKAYKKYRNELYEIYRKECEEKKLLQEILANEVIASGDVFNCNKMDFKFLQRNLGRKIQSASPAMLKSTIERKLSYHNVSINEISYTKLNQVFEERNVSKKNLEEVAALIRQF

SEQ ID NO: 281 - protein, 607 aa, unidentified
  REGION 1..607: note = uncultivated organism
  REGION 1..607: note = MG91B-131 Effector
MAKEEFVLTLPLKVEKWQADILDKRYELLRQVYNMVQQKLVRQYIYFSQQKAYRDCKNFEQRRGFFEAHPFHVKSVKGKNGELCDIKFPYTYKSRGGSSGKRAPNGISDYVSKLWSHKIGQNKTLADFGINSYIMEELGLHIMKAWEDRLYKPKSKRVSFKDYGEINSFGSRNKNGNFAGFTINLDNGIITYKTNGRQGQFADLIQLPIDFSREKNGYASEALKGGIESLKKLLIVRRCIRGENKYYLQLTISGEKPQKGRTLGKGNVGIDIGPSTIATSSLNGVYFDKLADKCDNIEHELYLINRKLDRSRRANNPQNYNEDGQIKRGVRLVWKKDKDVKRYATLKLQRKELYRKQAEIRKLQHLEMANAMLSEGDTFIVENNPIDAWVRRTKETTKTKAGKNRCKKRYGKSVANHAPAMFVSILKNKVLSLGGKFYEIDPRNAATQFDFSNGEFTKHEVSERSITLSNGRTHQRDMLSAFNLQHIVLQNKELKEYDIEQMEADYPIFCRLENTELERFKEARHKERDALYFAGASEKQMRRQPQEESVANRHNGGVGRKHCNTSLGGGNAKITGNGLARESIERVSQVPDERHLSGCVPISLDGL

SEQ ID NO: 282 - protein, 528 aa, unidentified
  REGION 1..528: note = uncultivated organism
  REGION 1..528: note = MG91B-132 Effector
MARKKVGEEARCVLTLPLYPEPYQLDVMEKRFRIFEELKRSLFYVELRKLKNLERTRAYRELQQQIRDEYKKQDSQSAKKLKALNARRNEMLKEAGLYQYRFQEDIKPMRAHFETHVYSSMAIAMADDVWKAFEKYLHGSGRQVHGVRRGSLSSVSGKGQWHEDKQSQALLGLDYRDGKLIWSGGGKKYRPDKDGQPGKRKDTREKKHTLEIRVAEPRTEYEREMLKKRICYCRIVRKWMKTRYKYYVQFVLDGNAYAKERVPVPGRVGIDIGPQSAAIVSENRAMLVELAERVDANDKKLHECQRRMDASRRSMNPENFRADGTVKSRRFRWKNSRHYLKLAGQARELQRKNADIRRYAHYCLANEVLRMGSEVYVENMDFRALQKRSKKTEKNEKGRFKRKKRFGKSLANKAPAMFLTILDNKLKSLCGTELHRVDTWSYRASQYDHLSRQYTKKALGERKRVLANGDRLQRDLYSAFLIMNADEERKSVDQQRCDKSYERFKELHDREIERIQNDGKKHLSSFGI

SEQ ID NO: 283 - protein, 521 aa, unidentified
  REGION 1..521: note = uncultivated organism
  REGION 1..521: note = MG91B-133 Effector
MMVTWKEGIPVGKSSTDTCCLTLPLKLEKWQEDRLAKRFEIARQIYNTMVRAELKKLERVKQLPEYRKNQKEIQTLDGKNSADRARLKELYGNQRKILQKQGFTEYGFKADSKDYYKHFIENIGSSVATHGIAPQVWNAFDKMLFQKDGKVVHFKRYGDVHSLKGGSVAHKSGGAEIMFRKTYIEWKGLKLPLKLSPNNAYEAEMLSYRVKYVRILRTRGKTKDRWYAQLSLEGKPAVKYDSSTGKPVHPVGRGAVGLDIGPQTLAYAAANEANLVELADQVQNIEQEKRRLQRKLDRSRRATNPDNYNENGTIRRGVKLTHNKSKRYCKLQKELSWIQHCQAETRKRQHIALANHLLSLGDCFYVEPMAWPSLTHRAKKTEVSEKTGRIKRKKRFGKSVANKAPASLIGILRLKCISLGLTGVVEVPMLLKASQYNHQSKDCAKKSLSQRWNDMPDGNRIQRDLYSAFLLQHYDSQSESFDQETLERDYPQFVRLHNQTIQRLSALPKTPSSMGIRRSVS

SEQ ID NO: 284 - protein, 621 aa, unidentified
  REGION 1..621: note = uncultivated organism
  REGION 1..621: note = MG91B-134 Effector
MRLFDYICIILLMIQSMIEEFVLTLPLKVEPWQADILNNRYEHLRRIYNMVQRKLLRHYIYLSQQNKYRQCKDNDEKRTFWETHHVTIKDFCDKKGNPAILRFPHTYYTRSKASGKRTSHGIPDLLPKLKNRNIGSGLTYASLGINAAILENLGLRIEQAWKKRLYDHKSKHISFKEEGDLNTFGCREKDGFSGFVLDLQKMKLSFNTNGRQGKSAKFIHFPINAGKELTEYEMYALKGGIGSVRKIIVAREYVRGRYKYYVQLTIKGEKPQKNRTLGKGTVGIDVGPSTVAVSSLHGVSLDKLADKCDNIEHDLWRINRKMDRSKRDTNPDFFNEDKTYKKPEKGERRKWTFSNRYKRLKLKRRELYRKQAAIRKRQHIEMANALLSQGDEFIVENNPIDSWTKRAKETKRGKNGKIRCKRRFGKSVAHHAPAMFVSILQNKVRSLGGTFHKVEIKNAATQFDFTNGEFTKHELNERTVTLSNGRTHQRDMLSAFNLQHLRTNSEELKDYDIEQMEADYPVFCKLEEKGIAQFKEKRQQERDVAYFTGVSHKTLKFSSANRHNGGVGRGNCNYHAGSKRNVAKTGNKSSSKENCESMETVSLVPNEYHHSNHQPFRLDGL

SEQ ID NO: 285 - protein, 495 aa, unidentified
  REGION 1..495: note = uncultivated organism
  REGION 1..495: note = MG91B-135 Effector
MPNTDSNRCILTLPLMTEPWQEHLIETRFDIMEHLKNSLIALELRKLKNLQRTRAYKELEKKIFEKPKEERKPLYKERSAMLRTAGFSEFDFINDMTPMQKHFADHIAAQISHRSASDVWRAFESYLFSSGREVHFQKRGTLDSVACKKAGNGMNYRNDTFEWSGGRTPNAKRLCIRVENPQTEYEKHMLEKEVKNMRIIRKWMKTRYKYYLQLTLVGDPYQKPRKIGEGRVGIDIGTRTIAIASETTVRLLELADRIDGNHKRKCELQRKMDASKRATNPDNFNSDGTIRRRSKGKKMTWHFSKHYLRMRDQVRELERKNADIRTYQHNCLANEVLTLGTDVFVEPMSYKGLQHRAKETKVDEKGRYLKKKRFGKSLANKAPSKFLMILENKLACKGKALNYVDINRFRASQFDHTNGTYRKKQLSERFATLSTGEHVQRDMYSAFLLMNSNDSLNAPDIERCGQTFERFKELHAIEVERIRQSPYRRIQSFGI

SEQ ID NO: 286 - protein, 524 aa, unidentified
  REGION 1..524: note = uncultivated organism
  REGION 1..524: note = MG91B-136 Effector
MEEVFVLELPLQVEKYQADILNKRYEQLRQLYNYVQGKLLRQYRYFEQMSEFQGCKTIREKRDFFRSHPFHIKGKLAGNKELPEITFDEYGINGFVEKLAQKSAGSNTTYADLGFTTYILDHLSTSIWAAWEKMLYDYRAKRISFKKYGELDSFSSRRKVVGGKVYFSGMELHLDTMELAIKLNAKQGKNAKFVTLRILHNPKHADYEMWALKGGLESIKVVKVVRRLVRGQYKYYLQMSIEGEKPKKGRTLGKGNVGIDLGPTTVAVSGENIASIDKLASKCDNIQEEITRLARKIDRSRRANNPQNFNEDGTIKRGVRLVWNDSKRYKALRREMAELQRRQAAIRKQQHIERANELLKEGDTFIVENNQISGWTRKAKETKVNEKTGKIQKKKRFGKSVANHAPSMFVTILENKVKSLGGEVIKVDTQNAASQYDFTNNSFEKHELKERSVTLSNGDTHQRDMLAAFNLQHLKYDKQEKKQYDTEAMAQHYERFCELEQEEIMRYKNKEKKDDRSTIGAGEL

SEQ ID NO: 287 - protein, 495 aa, unidentified
  REGION 1..495: note = uncultivated organism
  REGION 1..495: note = MG91B-137 Effector
MPFKPSNELQHDIEQGFKLLTIRYNTELKKLKKRYDYLIKTQEYEIAKREKHEKTFWEDNNFTKYDFYEFFFRINSFKRAQHPQQSIYDNTIVSSTMLRALGDHLLSSFDKLLYQNGKDVHPKKISSMTFRGLVQIRYNSQTQSFEFTHRGRRNIISQKVDIKTDYEIYAFNNPIKEVTIKKQIIRGKEKYFFHICFRGTPYNKGRELGTNSVGIDPSMLKMFACYSNGEMIEHSLLDKIDEKSGEICDSKYNSDKYEKLEKKIGVLQTQINRRRRIENPKFFKENGEINEEALNQARLEHPNSDPWKYSKATIKMRNKVAQIKRNLALHRKECHFRLANYILSKANTIKVENNSYKSFQARAKETTYNANGRPRSKKRFGKSIQKGAPSAFITILKNKAASWGDRVSFSEVGASEACTQFDHTNDEFTKHELKERVVELSNGDRVHRDFHAAMNILFFKNTVTKSKKGTKKDSDHFDIEGMREFYNQHRENMIT

SEQ ID NO: 288 - protein, 605 aa, unidentified
  REGION 1..605: note = uncultivated organism
  REGION 1..605: note = MG91B-138 Effector
MVEEFVLTLPLKVEPWQADILNNRYEHLRRMYNMVQRKLLRHYIYLSQQKAYRQCKDNDEKRTFWENQPITFKDLCDKNGNPAIVRFPHTYYTRSKASGKRTSNGIPDLLPKFKNRNIGSGLTYASLGINAAILENLGLRIEQAWKKRLYDPKSKHISFKDEGDLNTFGCREKDGFSGFVLDLPRMMLSFNINGKQKSQAKFIHLPINAGKELTEYEMYALKGGLDSIRKIIIAREYVRGRYKYYVQLTIKGEKPQKNRTLGNGSVGIDVGPSTVAISSLQGVTLDKLADKCDDIEHDLWRINRKMDRSKRKTNPDFFNDDKTYKKRKRGERRIWHFSKRYKRLKLERRELYRKQAAIRKQQHIDMANALLSQGDEFIVENNPIDSWAKCAKETKKNKNGKNRCKRRFGKSIAHHAPAMFISILHNKVRSLGGSFSKVEIKNAATQFDFTNGEFTKHELNERIVTLSDGRTHQRDMLSAFNLQHLRPESEEIKDYDIEQMEVDYPIFCKLEEQGIAIFKEKRPQDRDVAYFVGTSHKTLKISSANRHNGGVGRKNCNHHAGKRKNVAKAVTKSSSKKNCESLESVSVVPNEYHHSDNQHLRLDRL

SEQ ID NO: 289 - protein, 495 aa, unidentified
  REGION 1..495: note = uncultivated organism
  REGION 1..495: note = MG91B-139 Effector
MAGKSDKARCVLTLPLLTEPWQEHIIEKRFRIMEHLKNSLIALELRKLKNIQRTKQYRQLKAEISNAPKENRKTLDKKRKELLINAGFSERAFKDDMTNMQKHFVEHISTHAAHKSASDVWRSFDKVLFHTGRKIHFIRRGSLSSIACQTDGNGMRFRNGMLHWNGGRCKNKVKLELRVACPKTPYECAMLEKKIKFERIIRKWVKTRYKYYVQFTLVGNAVPKPRKTGTGRVGIDIGPSSIAIASSQEVRLMELADKINQNHLRKVALQQKMDRSRRATNPDNFNENKTIRRGVKLSWHYSKHYQKLRGQVRELERKNADIRKYQHTCLANYILSLGTEVYIEQMNFKGLQARAKETTYNKNGRANRKKRFGKSIANRAPAMLVTILETKLKSYAGTELHKVNTWSYRASQYDHITDTYTKKLLRQRWAELGNGDRIQRDLYSAFLLMNSQPSLQSTDRERCSKTYSQFKSLHDQEIYRLQQTPERHLSSFGIA

SEQ ID NO: 290 - protein, 528 aa, unidentified
  REGION 1..528: note = uncultivated organism
  REGION 1..528: note = MG91B-140 Effector
MNEVFVLELPLQIEKYQADALNKRYEQLRQLYNYVQGKLLRQYRYFEQMKEYQACKKIKEKRFFFRSHPFQINETLKGNKIPFKVSFDEYSIKGYIEKLVQKSVGPNMTYADLGFTTNILDHLGASVWAAWDKKLYDLKAKRISFKKYGELDSFSSRRKVVRGVVYFSGMELHLDSMELAIKMNGKVGKDAKFITLKMLHNPKHADYELWALKGGVDSVKVVKVVRRLVRSQYKYYVQLTIEGEKPQKGRTLGKGNVGIDLGPTTVAVSGVNVVSIDKLASKCDNIQEEITQLSRKIDRSRRANNPQNFNEDGTIKRGIKLVWSDSKRYKELRRELAELRRKQAVIRKQQHIERANALLKEGDTFIVENNPISSWTTRAKETKVNEKTGKIQKKKRFGKSVANHAPSMFVSILENKVKSLGGRFERVSTQNAASQYDFTTDTFKTGEEKHKLNERAITLSNGDTHQRDMLAAFNLQHLKYDEKDAKQYDTEAMAADYERFCQLEQEEILRYKNKVKKDDRGTIGAEKL

SEQ ID NO: 291 - protein, 495 aa, unidentified
  REGION 1..495: note = uncultivated organism
  REGION 1..495: note = MG91B-141 Effector
MQKENKNGCCILQLRLKPEPWQVDVIETRFRILEHLKNSLTAYELRKLKNLERTREYRAILEALKIADEKTKKNLYKKRQKMLTDAGFSEFRFKDDITPMQKHFVEHISTHVAHRAASEVWRAFDKFLFGSGKTVHFVRRGTLKSAANQKAGTSMAMKNGYFEWNGGRSSNTISLKVRIAKPETYYEKEMLQKKAKYFRIVRKWGKTGYKYYLQITLEGQPVLKPRAIANGKKVGIDIGPSSIAIVSDKKVELRQLADHVQNNYEEKCLLQRSMDRSRRNLNTQNYNEDGTIRRGIRLEWKLSKHYMEMRGKVRMLERKNADIRKYQHTCLANEILSLGTNVYIEQMSFKGLQRKAKETTYREDGRPKRKKRFGKSIANRAPAMFVEILNKKLETIVGVELNKVDTFQFKASQYDHTNGSYKKKLLKERWAQLSNGDRIQRDLYSAFLLMNSNNDMNSADVDKCNATYIEFKKLHDKCIDKLKTDGGIRLPSFGI

SEQ ID NO: 292 - protein, 523 aa, unidentified
  REGION 1..523: note = uncultivated organism
  REGION 1..523: note = MG91C-1 Effector
MEKELFILELPLKVEKWQEDLLNKRYEYLRQIYNYAQGKLLKEYNYLSQMADFKNCKTKKAKSEFFNTHPFIIKGFKKPVIFSKMKSKEIEADVYGITDFIEKIVQKKVDNQKTYKDFGINTTMINALANHIWAAWEKVIYGNGKHISFKKYGDINTISIRKKGNFNGLDVDINNMFLAFNINGRKRSNSKKIKLLIDYKKLTDYEIYSLKNGYDSIRVVTIARKLIRGKYKYYVQFTIEGEKPQKGRTLGKGNVGIDVGPSTIAVSSLKGVSIDTLSEKCNDIEHDLYIINRKIDRSRRATNPQNFKEDGQIKPINRREGQKRHWNKSKHYVNLSNKKRELLRKQAAIRKLQHNIKANELLTLGDTFIVENNPISAWVKKSKETKINKKGKFQSKKRFGKSVANHAPAMLITILENKIKSLGGKFIKVDIKNAASRYDFTNNEFKEHGLNERKITLSNGNTHQRDLLAAFNLQHIDLTTEEFKKYDVNEMVKDYPIFCNLEKEEIQRFLDGLKKRHNSTIGI

SEQ ID NO: 293 - protein, 544 aa, unidentified
  REGION 1..544: note = uncultivated organism
  REGION 1..544: note = MG91C-2 Effector
ISLIVFNIKYLVVSKFWFTFANEIRKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEDGQIKRIDRKKGEKRVWKYSKRYVKLRNKKRELLRKQTVIRKLQHNIKANEILSLGDTFIVENNPISAWAKRANGVRKNKNGKIQTNKRLGKSIGNHAPAMFVTILQNKVESLKGKFVQVDLKNAASRFDFTNGDFTEHNLDERKITLSNGNTHQRDMLAAYNLQHIDLTNDYLKKYNVDEMIEDYPIFCKLEKEEIKRFIDKMKISNKSNIGL

SEQ ID NO: 294 - protein, 523 aa, unidentified; identical to SEQ ID NO: 279
  REGION 1..523: note = uncultivated organism
  REGION 1..523: note = MG91C-3 Effector

SEQ ID NO: 295 - protein, 509 aa, unidentified
  REGION 1..509: note = uncultivated organism
  REGION 1..509: note = MG91C-4 Effector
MNKRIKEDGKENFILCLPLQVTKQQADILNKRLSICCGIYNNFQKKMLRKFNYVKQMKAYKECKTLSERRKFLNGYTIQWKNTKGKLYDLKPFTEFGFKSYIAVFNETFSNNGINSKLLQYIASNAWSAWDKVLYGKGKRISFKKRDEFNSYTIGISSNNTFNGIDISKINENKIGVNLNGGRGKNAKVIYIPYKINTDYERECFLNDIRAIWLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 296 - protein, 523 aa, unidentified
  REGION 1..523: note = uncultivated organism
  REGION 1..523: note = MG91C-5 Effector
MKSEKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEYGQIKRIDRKKGEKRVWKYSKRYVKLRNKKRELLRKQTVIRKLQHNIKANEILSLGDTFIVENNPISAWAKRANGVRKNKNGKIQTNKRFGKSIGNHAPAMFVTILQNKVESLKGKFVQVDLKNAASRFDFTNGDFTEHNLDERKITLSNGNTHQRDMLAAFNLQHIDLTNDDLKKYNVDEMIEDYPIFCKLEKEEIKRFIDKMKISNKSNIGL

SEQ ID NO: 297 - protein, 509 aa, unidentified
  REGION 1..509: note = uncultivated organism
  REGION 1..509: note = MG91C-6 Effector
MNKRIKEDGKENFILCLPLQVTKQQADILNKRLSICCGIYNNFQKKMLRKFNYVKQMKAYKECKTLSERRKFLNGYTIQWKNTKGKLYDLKPFTEFGFKSYIAVFNETFSNNGINSKLLQYIASNAWSAWDKVLYGKGKRISFKKRDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 298 - protein, 521 aa, unidentified; identical to SEQ ID NO: 228
  REGION 1..521: note = uncultivated organism
  REGION 1..521: note = MG91C-7 Effector

SEQ ID NO: 299 - protein, 511 aa, unidentified; identical to SEQ ID NO: 274
  REGION 1..511: note = uncultivated organism
  REGION 1..511: note = MG91C-8 Effector

SEQ ID NO: 300 - protein, 601 aa, unidentified
  REGION 1..601: note = uncultivated organism
  REGION 1..601: note = MG91C-9 Effector
MIEEFVLTLPLKVEKWQADILNNRYEHLRRIYNMVQRKLLRQYIYYSQQSEYRKCKSNDEKRDFWENHPFTIKGIYDQKGNSSIVKFPYTYSSRSKNSGKRASNGISDLLPKLKHYNIGGGKTYGSLGLNASILENLGLSVQQAWEKRIYNSKSKHISFKKEDELNTFGCREKDGFSGFVLDLSKMELSFNTNGLHGKKAEFIHLPINPNKKFTQYEVEALKGGPESVRKIVISREYIRGAYKYYVQLTIRGEKPQKNRTLGKGTVGIDVGPSTVAVSSLYGVALNKLADKCDGIEHDLWLVNRKMDRSRRATNPDFFAEDGTYIKRKKGERRIWNFSNRYKNLKSVRRELYRRQAVVRKQQHIAMANEMLSQGDTFIVENNPVDSWTRRAKETKKNKKGKMRCKRRFGKSVAHHAPAMFVSILQNKVRSLGGEFHKVDIKIAATQFDFTNGEYKKHELKERTVTLADGKTHQRDMMAAFNLQHFRADSSEDYDIEQMILDYPVFCKLEEQGIANYIESKGEEKDTAYFAGKSSKELKRSSNRHNGGVERKHCNIHERKGDAAESGKKTLFGGISESLESVPDVPNECHHLGHQHLRLDEP

SEQ ID NO: 301 - protein, 621 aa, unidentified; identical to SEQ ID NO: 284
  REGION 1..621: note = MG91C-10 Effector
  REGION 1..621: note = uncultivated organism

SEQ ID NO: 302 - protein, 509 aa, unidentified
  REGION 1..509: note = MG91C-11 Effector
  REGION 1..509: note = uncultivated organism
MNKRIKEDGKENFILCLPLQVTKQQADILNKRLSICCGIYNNFQKKMLRKFNYVKQMKAYKECKTLSERRKFLNTYTIQWKNTKGKLYDLKPFTEFGFKSYIAVFNETFSNNGINSKLLQYIASNAWSAWDKVLYGKGKRISFKKKDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSRRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 303 - protein, 523 aa, unidentified
  REGION 1..523: note = MG91C-12 Effector
  REGION 1..523: note = uncultivated organism
MKSEKKNRCMEKEMFVVELPLKVEKWQSDVLNKRYELIAKIYNYAQKKLLRQYIYLVQMDEYKKCKTKKEKSIFFKNHPFYFKDITSKITFSEYDVTAFVEKIIKKNVSSDKTYKDFGINTSITECIGKHIWSAWEKKIYGNGKKVSLKNKGELNTISIRKKSEYFGGLDIDIEHMILSFNINGKTKSKAKKIILPINVKNLTEYEMYALKDGYDSINVVTIIRKMIRGNYKYYVQFTIEGEKPQKGRTLGNGNVGIDIGPSTVAISSLNGVTIDTLAEKCDNIESKISNIERKMDRSRRATNPQNFTEDGQIKRIDRKKGEKRVWKYSKRYVKLRNKKRELLRKQTVIRKLQHNIKANEILSLGDTFIVENNPISAWAKRANGVRKNKNGKIQTNKRFGKSIGNHAPAMFVTILQNKVESLKGKFVQVDLKNAASRFDFTNGDFTEHNLDERKITLSNGNTHQRDMLAAFNLQHIDLTNDDLKKYNVDEMIEDYPIFCKLEKEEIKRFIDKMKISNKSNIGL

SEQ ID NO: 304 - protein, 509 aa, unidentified
  REGION 1..509: note = MG91C-13 Effector; note = uncultivated organism
MNKRIKEEGKENFILCLPLQVTKQQADILNKRLSICCGIYNNFQKKMLRKFNYVKQMKAYKECKTLSERRKFLNTYTIQWKNTKGKLYDLKPFTEFGFKSYIAVFNETFSNNGINSKLLQYIASNAWSAWDKVLYGKGKRISFKKKDEFNSYTIGISSNNTFNGIDISKINENKIGVNLNGGRGKNAKVIYIPYKINTDYERECFLNDIRAIGLKRVFIRGKWKYFITFSFEGKKPVKNRKLGKGKVGIDIGPSTIAISSDDVVYMDVLAKNVNSIEDKKRLLQRKIDRSKRKTNPLQFKEDGSIRRYKKGERPKWIISNHCKDYYVKLKELYRKQSVYRKNSHIELSNMLLNLGNDFIVENNPVEAWMRKSKEITFNKKGKINSRKRFGKSIANHAPSMFITILENKVKSLGGSLTKADIKNAASQFDFTNETFTKHELKERRINLSNGNTHCRDSIAAFNLQHLIGDKEYDVSTMKEKYNKFCRMEQEEIKRHQMFDEKISSNFGIN

SEQ ID NO: 305 - protein, 521 aa, unidentified; identical to SEQ ID NO: 166
  REGION 1..521: note = uncultivated organism; note = MG91C-14 Effector

SEQ ID NO: 306 - protein, 605 aa, unidentified
  REGION 1..605: note = MG91C-15 Effector; note = uncultivated organism
MIEEFVLTLPLKVESWQADILNNRYEHLRRIYNMVQRKLLRHYIYLSQQNEYRQCKDNDEKRTFWENHPITFKDFCDKNGNPAIVKFPYTYSHRSKDSGKKASNGISDFVPKFKNRNIGGGLTYGSLGINASILENLGLRIEQAWKKRLYDPKSKHISFKEEGDLNTLGCREKNGFSGFVLDLQKMTLSFNTNGRQGKSAKFIHFPINAGKELTEYEMYALKGGIGSIRKIVVSREYVRGRYKYYVQLTIKGEKPQKNRTLGKGTVGIDVGPSTVAVSSLHGVSLDKLADKCDNIEHDLWRINRKMDRSKHETNPDFFNEDKTYKKPEKGERRKWTFSNRYKRLKLERRELYRKQAAISKQQHIDMANALLSQGDEFIVENNPIDSWAKRAKETKRGKNGKIRCKRRFGKSVAHHAPAMFVSILQNKVRSLGGTFHKVEIKNAATQFDFTNGEFTKHELNERTVTLSNGRTHQRDMLSAFNLQHLRPDSEETKDYNIEQMEADYPVFCKLEEKGIAQFKEKRQQERDVAYFTGVSHKTLKFSSANRHNGGVGRKNCNHHAGSKRNVAETGNKSSSKEYCESMETVSLVPNEYHHSNHQHLRLDGL

SEQ ID NO: 307 - protein, 461 aa, unidentified
  REGION 1..461: note = MG91C-16 Effector; note = uncultivated organism
MEKEMFVVELPLKVEKWQSDVLNKRYELLAKIYNYAQKKLLRQYIYLTQMDEYKKCKTKKDKSVFFKNHPFYFKGITSKITFSEYDVTAFVEKIIKKNVSCDKTYKDFGINSSITEYLGKHIWSAWEKKIYDNGKKVSFKNKGELNTISIRKKSEYFGGLDADIEHMILSFNINGKKNSKAKKIILPINVKNLTEYEMYALKDGYDSINVVTIIRKMIRGKYKYYVQFTIKGEKPQKGRTLGKGNVGIDIGPSTVAISSLNGVTIDTLAEKCNNIEDKISDIERKMDRSRRATNPQNFTEDGQIKRINRKKGEIREWKYSKRYVKLRSKKQELLRKQAAIRKLQHNIKANEILSLGDTFIVENNPISAWAKRAKGVRKNKNGKIQTNKRFGKSIGNRAPSMFVTILQNKVKSLKGKFVPVNPKNAASRFDFTNGDFTEHNLDERKITLSNGNTHQRDMLAA

SEQ ID NO: 308 - protein, 518 aa, unidentified
  REGION 1..518: note = MG91C-17 Effector; note = uncultivated organism
MNNKKEDDIFVLELPLSVEKWQSDLLDRRYEYLRQIYNYAQRKLIRQYIYLVQTEEYKKCRTKKDKSSFFGNHEFWFNGIKCPITFTEFGIKKYVTKLCKLKVSSGMTYKDFGLNSSHLETLGANMWAAWDKKIYGKGERINIKRYGCLNTFSVRCKSGYFGGLKVILDGNEIYLFLDMNGKKGEKEKKEIKLTVKIGKMTEYEMQALRNGIENIRMVTIARKLVRGKYKYYVQFAIKDEKPKKGRKLGKGSVGIDVGPSTVAVSSLHGVEINTLAEKCDDIHRKVRLIQRKMDRSSRASNPQNYNEDGTIKKVRGQKLKWVRSKNYVRLRNEKRELMRKQAAIRKLSHNIDTNKLLELGDTFVVEDNPISSWTKRASGIRKGKNGKNLSNKRYGKSVANHAPAAYVEILKNKVESLGGKFIKVDTKNGASGYDFTTGEYTKHGVEKRKVTLSNGNVHQRDMLAAFNLQHLNYEDGELKSYNVEEMEKDYPIFCKLEKEEIKRYTSGQKTSHKSTVGY

SEQ ID NO: 309 - protein, 630 aa, unidentified
  REGION 1..630: note = MG91C-18 Effector; note = uncultivated organism
MILVHFSLHHEICEFKFYFVNLQEIFSTPMIEEFVLTLPLKVELWQADILNKRYEHLRRIYNMVQRKLLRNYIYLSQQDAYRQCKDNNEKRDFWENHPVTIKEFCDKNGNPAIVKFPYTLSRRSKNSGKKASNGISDFVPKFKNHNIGSGLTYGALGINASILEELGLRIQQAWEKRLYNPKSKHISFKEEGDINTFGCREKRGFSGFVLDLSHMMLLVNINGRQHQLAQYLNLPINPNKELTEYEMYALKGGLSSVRKIIISREYIRGRYKYYVQLTIKGEKPQKNRTLGKGTVGIDVGPSTVAVSSLQGVSLDKLADKCDDIERDLWRINRKMDRSKRATNPDFFNKDKTYSKRKKDERRKWTFSNRYKRLKLERRELYRKQAAIRKQQHIEMANALLSQGDEFIVENNPIDSWAKRAKETKKGKNGKNRCKRRFGKSIAHHAPAMFVSILQNKVRSLGGSFREVEIKNAATQFDFTNGEFTKHELNERTVTLSNGRTHQRDMLAAFNLQHLKEEVKDYDIEQMDVDYPLFCKLEEKGIAQFKENRHLDRDVAYFVGASHKTLKFSSANRHNGGVGRKNCNHHSGGRDVAKTGNKSSIMKNCESLEPVSVLPNEYRHSNHQHLKIDGL

SEQ ID NO: 310 - protein, 549 aa, unidentified
  REGION 1..549: note = uncultivated organism; note = MG91C-19 Effector
MLCNTIIMVLTGCALKNLATEGVLCNAITMALTSFTHDIQFFPYITRYKTLSNMEEIFILEMPLHVEQWQADLLNKRYEYLRKLYNYVQSKLLKQYEYFIQMNEYKECTGSCKTPLFEFFKTHSFHFRGINNSQGLPLPITFSKMGICAYVCQLAKKNIGDNTTYKDVGINTCVLKHMGEHIWAAWDKMIFNKKTNRISFKKYGEMNILGAIKSGGYTCGFDIDLENFILTFNLNGKQGKNGKYIHLPILPNVKHADYETEALKGGYNSIRVLTIVRKLIRGKYKYYLQITIKGKKPQKNRTLGKGNVGIDIGPSTIAISCDSKVDVSQLADKCENIQHKIAVINRKLDRSRRATNPDNFNENGTYKPINRKNGERRTWYYSNRYKKLKAIRQELQRKQAAIRKLQHTEYVNTLLPLGNTFIVEKNPYCGWAKRSEGIRYDKNGKICSNKRYGHSIGIHAPSTLITLLENKVKSLGGKMFKVDIRNAATGYDFTNGEYTKHDISDRKITLSNGDVHHRDIMAAFNLKHLNYDSKELKDYNIPEMYQDYD

SEQ ID NO: 311 - protein, 630 aa, unidentified
  REGION 1..630: note = uncultivated organism; note = MG91C-20 Effector
MILVHFSLHHEICEFKFYFVNLQEIFSTPMIEEFVLTLPLKVELWQADILNKRYEHLRRIYNMVQRKLLRNYIYLSQQDAYRQCKDNNEKRDFWENHPVTIKEFCDKNGNPAIVKFPYTLSRRSKNSGKKASNGISDFVPKFKNHNIGSGLTYGALGINASILEELGLRIQQAWEKRLYNPKSKHISFKEEGDINTFGCREKRGFSGFVLDLSHMMLLVNINGRQHQLAQYLNLPINPNKELTEYEMYALKGGLSSVRKIIISREYIRGRYKYYVQLTIKGEKPQKNRTLGKGTVGIDVGPSTVAVSSLQGVSLDKLADKCDDIERDLWRINRKMDRSKRATNPDFFNKDKTYSKRKKDERRKWTFSNRYKRLKLERRELYRKQAAIRKQQHIEMANALLSQGDEFIVENNPIDSWAKRAKETKKGKNGKNRCKRRFGKSIAHHAPAMFVSILQNKVRSLGGSFREVEIKNAATQFDFTNGEFTKHELNERTVTLSNGRTHQRDMLAAFNLQHLKEEVKDYDIEQMDVDYPLFCKLEEKGIAQFKENRHLDRDVAYFVGASHKTLKFSSANRHNGGVGRKNCNHHSGGRDVAKTGNKSSIMKNCESLEPVSVLPNEYHHSNHQHLKIDGL

SEQ ID NO: 312 - protein, 516 aa, unidentified
  REGION 1..516: note = uncultivated organism; note = MG91C-21 Effector
MEKKKEMFVVELPLKVEKWQSDVLNKRYELITKIYNYAQKKLLRQYIYLTQMDDYKKCKTKKDKSVFFKNHPFYFKGITSKITFSEYDVTAFFEKIIKKNVSSDKTYKDFGINTSITGYLGKHAWSAWEKKIYGTGKKISFKNKGELSTISIRKKSEYFGGLDVDIEHMILSFNINGKTKSKAKKIILPINTKNLTEYEMYALKGGYDSIKVVTIVSKMIRGKYKYYVQFTIEGEKPQKGRTLGKGNVGIDIGPSTVAISSLNGVTIDTLAEKCDNIEDKISDIERKLDRSRRATNPQNFTEDGQIKRIDRKKGEKRKWEYSKRYVKLNNKKRELLRKQAAIRKLQHNIKANEVLSLGDTFIVENNPTSAWAKRAKGVRKNKNGKIQTNKRFGKSIGNHAPAMFVTILQNKVESLKGKFVKVDIKNAASRFDFTNGDFTEHNLDERKITLSNGNTHQRDMLAAFNLQHIDLTNDDLKKYNVEEMMEDYPIFCKLEKEEIKRFTNGMKIPNKSNIGI

SEQ ID NO: 313 - protein, 510 aa, unidentified; identical to SEQ ID NO: 177
  REGION 1..510: note = MG91C-22 Effector; note = uncultivated organism

SEQ ID NO: 314 - protein, 466 aa, unidentified
  REGION 1..466: note = uncultivated organism; note = MG91C-23 Effector
QMDEYKKCKTKKDKSAFFKNHPFYFKGITSKITFSEYDVTAFVEKIIKKNVSSDKTYKDFGINTSITECLGKHIWCAWEKKIYGKGKKVSFKNKGELNTISIRKKSEYFGGLDVDIEHMILSFNINGRTKTKAKKIILPINVKNLTEYEMYALKGGYDSINVVTIIRKMIRGKYKYYVQFTIEGEKPQKGRTLGKGNVGIDIGPSTIAISSLNGVTIDTLAEKCDNIEGKISDVERKMDRSRRVTNPQNFTEDGQIRRIDRKKGEKRVWKYSKRYIKLRNKKRELLRKQTVTRKLQHNIKANEILSLGDTFIVENNPMSAWAKRAKGVRKNKNGKIQTNKRFGKSIGNHAPAMFVTILQNKVESLKGKFVKVDPKNAASRFDFTNGDFTEHNLDERKITLSNGNTHQRDMLAAFNLQHIDLTNDDLKKYNVDEMIEDYPIFCKLEKEEIKRFINGMKKSNKSNIGI

SEQ ID NO: 315 - protein, 607 aa, unidentified
  REGION 1..607: note = uncultivated organism; note = MG91C-24 Effector
MIEEFVLTLPLKVETWQADILNKRYEHLRCIYNMVQRKLLRHYIYLSQQNEYRQCKDNDEKRTFWENHPITFKDFCDKNGNPAIVKFPYTYSHRSKDSGKKASNGISDFVPKFKNRNIGGGLTYGSLGINASILENLGLRIEQAWKKRLYDPKSKHISFKEEGDLNTLGCREKDGFSGFVLDLQKMMLSFNTNGRRHGSAEYIHLPIKPSKLTKYEKFVIDALKDKLSSVRKIVVAREYVRGKYKYYVQLTIKGEKPQKNRTLGKGTVGIDVGPSTVAVSSLQGVSLDKLADKCDNIEHDLWHINRKMDRSKRDTNPDFFNEDKTYKKPEKGERRKWTFSNRYKRLKLERRELYRKQAAIRKQQHIEMANALLSQGDEFIVENNPIDSWAKRAKETKRGKNGKIRCKRRFGKSVAHHAPAMFVSILQNKVRSLGGSFHKVEIKNAATQFDFTNGEFTKHELNERTVTLSNGRTHQRDMLSAFNLQHLRPDSEETKNYNIEQMEADYPVFCKLEEKGIAQFKEKRQQERDVAYFTGVSHKTLKFSSANRHNGGVGRGNCNYHAGSKRNVAKTGNKSSSKENCESMETVSLVPNEYHHSNHQHLRLDGL

SEQ ID NO: 316 - protein, 589 aa, unidentified
  REGION 1..589: note = MG91C-25 Effector; note = uncultivated organism
MSKSYEKCLWTAFLLGSDALLLKYYDYLCTLKLCSPIMIEEFVLTLPLKVESWQADILNNRYEHLRHIYNMVQRKLLRHYIYLSQQNEYRQCKDNDEKRMFWETHPVIIKDFCDKKGNPTIVKFPHTYYTRSKASGKRTSNGIPDLLPKLKNRNIGSGLTYASLGINAAILENLGLRIEQAWKKRLYDLKSKHISFKEEGDLNTFGCREKDGFSGFVLDLQKMKLSFNTNGRQGKSAKFIHFPINVGKELTEYEMYALKGGIGSVRKIVVAREYVRGRYKYYVQLTIKGEKPQKNRTLGKGTVGIDVGPSTVAVSSLQGVSLDKLADKCDNIEHDLWRINRKMDRSKHETNPDFFNEDKTYKKPEKGERRKWTFSNRYKRLKLERRELYRKQAAIRKQQHIEMANTLLSQGDEFIVENNPIDSWAKRAKETKRGKNGKIRCKRRFGKSVAHHAPAMFVSILQNKVRSLRGTFHKVEIKNAATQFDFTNGEFTKHELNERTVTLSNGRTHQRDMLSAFNLQHLRPDSEETKDYNIEQMEADYPVFCKLEEKGIAQFKEKRQQERDVAYFTGVSHKTLKFSSANRHNGGVG

SEQ ID NO: 317 - protein, 510 aa, unidentified
  REGION 1..510: note = MG91C-26 Effector; note = uncultivated organism
MFVVELPLKVEKWQSDVLNKRYELLAKIYNYAQTKLLRQYIYLTQMDEYKKCKTKQDKSVFFKNHPFYFKGITSKITFSEYDVTNFVNKIIKKNVSSDKTYKDFGINTSITEYLGKHAWSAWKKKIYGNGKKVSFKNKDELNTISIRKKSEYFGGLDVDIEHMILSFNINGRTKTKAKKIILPINVKNLTEYEMYALKDGYDSINVVTIMRKMIRGNYKYYVQFTIEGEKPQKGRTLGKGNVGIDIGPSTVAISSLNGVTIDTLAEKCDNIEDKISDIERKMDRSRRATNPQNFTEDGQIKRIDRKKGEKREWKYSKRYVKLHNKKRELLRKQAAIRKLRHNIKANEILSLGDTFIVENNPTSAWAKRAKGVRKNKNGKIQTNKRFGKSIGNHAPAMFVTILQNKVESLKGKFVKVDPKNAASRFDFTNSDFTEHNLDERKITLSNGNTHQRDMLAAFNLQHIDLTNDDLKKYNVNEMIEDYPIFCKLEKEEIKRFINGIKISNKSNIGI

SEQ ID NO: 318 - protein, 402 aa, unidentified
  REGION 1..402: note = MG91C-27 Effector; note = uncultivated organism
SITECLGKHIWSVWEKKIYDNGKKVSFKNKGEVNTISIRKKNEYFGGLDVDIEHMILSFNINGKIKAKAKKIILPINVKNLTEYEMYALQGGYDSINVVTIIRKMIRGKYKYYVQFTIEGEKPQKGRTLGKGNVGIDIGPSTVAISSLNGVTIDTLAEKCDNIESKISDIGRKMDRSRRATNPQNFTEDGQIKRIDRKKGEKRVWKCSKRYIKLRNKKRELLRKQTVIRKLQHNIKANEILSLGDTFIVENNPISAWAKRAKGVRKNKNGKIQTNKRFGKSIGNHAPSMFVTILQNKVKSLKGKFVTVNPKNAASRFDFTNGDFTEHNLDERKITLSNGNTHQRDMLAAFNLQHIDLTNEDLKKYNVHEMIEDYPIFCKLEKEEIKRFINGMKMSNKSNIGI

SEQ ID NO: 319 - protein, 723 aa, unidentified
  REGION 1..723: note = MG126-3 Effector; note = uncultivated organism
MAEEKEMRSYNIRINPDADAIKVIERDMKLMEQLYGFIIYKIESHIQQLRDETEYGEYDSNYKEYCRLMAEAEQKKRQCEVLRDAKKRKNIEDPELDSQYKELSAEIIKLDKHRKEWCRKCCEFFKNNKKALCHFVFESRRKVIRLKSDMVTPTENSGFILHHGVNGELYYIYDKTYYMMVEDSIRKANKGDGYSALVCECGVVTKRVSLNEDKTFKDKVYDGVAVLTDDGEIIMPYEYLRIDKLNKNNAHSFSILFVGEYVYSLNKNPKTKKSQKTSMAYKDFGFTSTDFFENIGNRLTLSYADFPKLLSQKHRKNDFMTIMCGYQSKLMDIMCDESGGYFVRMVTHSYGRGKKKYVDIPVLVSHRNKNRIGTERDMDTKVLEKKLKLLETGKTNVIGISKDVIREKSRYYVNIFLEHTGSLGLEYNENSLNQELGVGSVGVDPNMHQIAVYSDSLKKSTIFSLETIRGGIFKQKIYDCYSKLNSLQTKMSRCMDENNSGAKDENGNFIKGKKLFKSKRYLAMKKEVGELHRRIAELKETANIFIAHELLSYGNVFYIEDPRSAEKKRRMQGVVYDVKGRVVKNRKFSRSIQKFSISGMIYKALDVIESRGGKVYWINNKSAATAYVPKGDNVSHGMGERVFNWLGYKVQRDMKSGYVLSGCKELEQTKETYLNKPIYKVVSYDDERLLNDVDVFAESVKEEMERCRDLNVEDKTIGVEKYF

SEQ ID NO: 320 - protein, 572 aa, unidentified
  REGION 1..572: note = MG126-3 Effector; note = uncultivated organism
MPANVYQYGSQLKTVLTGEAEKQIRLQARFWNDLVEIEHEFSQRYRDVMNNADEKISSLNEEVGRRREEIEALRGEIKERRKKARSGRVDDSEIKKKIAELLAENKHLIQELKDYRAEVKIMVKPRLHELESERRARVKELRQQYATEGLYWGNYNAVLTSYQTARSRSMQSGGELRFRRFDGTGRLTCQIQGGMSVEEAFSGANNLFQIDPVPETAWNIPSRGESRKLCRTKAKIRITSDEEKNPVWIEIPIVMHRPIPQNARIQRVSISTKKVADRIRWFLNVTVIEDEPEKDDFAIGQVVALDVGWRKKEDGSIRVAYWIDSNGDTGEVELDVSFIHTDQRIRTLQKKRDDNFNVAKEKLSKWVSARKEHEGVPEWFTERTDTLAQWRAQSRLAALILFWRDNRFTGDGEVFSFLESWRRQDRHLWIWQANLRDKMSGRRLQQYRVFAAKAAKQYDAVIFEEFDLRSIKKKKRPEQGVDSDSSLRNFMKIAGVSILRSEVRRAFEATGKLFVKEQTSGQGACPFCAETAEREPGVGMFVSCTGCGKVYDRDWAKAKDLLSRWFVATKKL

SEQ ID NO: 321 - protein, 552 aa, unidentified
  REGION 1..552: note = MG126-4 Effector; note = uncultivated organism
MFGNKSLPSRIFSYGANAPIEGLDLVNEQMKLAHRYRNSLVELEHSRRKRVNEALARLSPRLVEVEELLIVADQKVEDAADAIRKASGAARKKVRPPEMVAEMKQCKEVLKALRADRKSLRTLLFASKAWEQEQDTLNAWVTEQQKALRAASGLYWGTYLHVEQSMAGTKSGPPPRFYRYDGTGHLAVQLQKGLSPKEAQENDPRIRIEVVPEGVYRPGKNRPKKLGNAIVHFRVGSDKSGDPIFARIPFILHRPLPEDSRIKWVHLISDRVGTHSKWLVQFVLSKESWPKADRATSGHVGVDVGWRMKEDGSLRVAVWKGSDGQDGELTLPLNWLPAMGKTRHIRSVRDKNFDTIRPELVAWLKERQDLPEWMKEPLTTMHMWKAQARLASFMIRWRTERFPGDEQMFQKAEDWRKRDRHLYDYESNLRDQLLAQRSDLYRNFAAKLRRLYKTAALEDLDLRDFHVLPPVEDPMMDPALKEHVRDACLSSLLQYLKESMGGFIEVPAANTTKKCHSCGSIQEWDHKILRHTCTKCGKDWDQDYNAAINIGA

SEQ ID NO: 322 - protein, 612 aa, unidentified
  REGION 1..612: note = MG126-5 Effector; note = uncultivated organism
MTARIYEYGLLAPSQEGERVSDQIWRAHRYRNLLCEIERRRREAVAAVLAAHPTIEPLSAAVDLLATEAEAADLAIRAARKASRSRSDTAAGRSALRDLRVRLKRARAEMRDAKRALRDDAPIQAQIAAVNEVDHCARLKARAECGVHWGSYLLIENAAIQAAKTAIKERTMPQFSAWDGNGAVGVQIQGGISVAEVHAGTDTRLQIDAVPEAAWTGTRGDRRRLGRTTLRMRVGSEGRAPIWAVWPMVMHRPLPAGAVLKGARVIRRRHGWRSRGSHDTWHLQITVVLPDEYRTEPCGTGMVAVHPGWRQVAGGLRIGYVLGDDGHERELLLDDSVAPRIASADAIHGHRDDAIEALRPALSAWLAGRDLPEWLAEQSRYLPMWRATSRFARLEARWRTERFAGDDEGYAMLDSWRRRDAHLYAYEDGRRDRALGHRREVYRRLAAELARRYEVLVLSDTDYRQLARRPRTEDERAAWDAANGQRHLAAVSTLTEACRQAFRARGGRIVTVASLDLTRTCHVCGAIVSWDQAAYVYATCACGATWDQDANAAHNSLARGREQLIADLAAEAARAKDPVVVESRWAKVKRLGREKREAARAADDNSIDVQRD

SEQ ID NO: 323 - protein, 612 aa, unidentified
  REGION 1..612: note = uncultivated organism; note = MG126-6 Effector
MTARIYEYGLRAPTQEGARVSDQIWLAHRYRNLLCEIERRRREAVAAVLAAHPTIEPLSAAVDLLATEAEAADLAIRAARKTSRSRSDTVADRSALRDLRVRLKRARAEMRDAKRALRDDAPIQAQIAAVNEVDHCARLKARAECGVHWGSYLLIENAAIQAAKTAIKERTMPQFSAWDGNGAVGVQIQGGISVAEVHAGTDTRLQIDAVPEAAWTGTRGDRRRLGRTTLRMRVGSEGRAPIWAVWPMVMHRPLPAGAVLKGARVIRRRHGWRSRGSHDTWHLQITVVLPDEYRTEPCGTGMVAVHPGWRQVAGGLRIGYVLGDDGHERELLLDDSVAPRIASADAIHGHRDDAIEALRPALSAWLAGRDLPEWLAEQSRYLPMWRATSRFARLEARWRTERFAGDDEGYAMLDSWRRRDAHLYAYEDGRRDRALGHRREVYRRLAAELARRYEVLVLSDTDYRQLARRPRTEDERAAWDAANGQRHLAAVSTLTEACRQAFRARGGRIVTVASLDLTRTCHACGAIVSWDQAAYVYATCACGATWDQDANAAHNSLARGREQLIADLAAEAARAKDPVVVESRWAKVKRLGREKREAARAADDNSIDVQRD

SEQ ID NO: 324 - protein, 678 aa, unidentified
  REGION 1..678: note = MG126-7 Effector; note = uncultivated organism
VKRKTSETPTRIYSYGCLLPIAGAELLEDQLRLANRYYNKLIEIERRRREAVDKITLSWSDDLGALDTAAQQALRLEEQYAIQKKDYGDKAPESAAMREIMRVARAELLCQAYELILSRRSEQSVSNLEALDKRVKSINEMLAVARAEVKALRAGKEKKVKSVNTKAAAARVKQLIQDQKAAKTDLQRERERLRTPALRCAQRAIGDIAGAEIRSARAACGLYSATYLKTEEAAGQAAKTSYPGLPHFQRYDGSGKIAVQLVGGISVKELFSGEDTQIRIKPVPEDTFYRRRHHRRLASRTVVKIRAGSNTDRTPKWVDLPIILHRPLPDDAVIKWAWIFRRRIGCRHEYRLQLVFESNTFRTGVQKAGRGQATIAIDLGWRKLEDGEIRVAYWLDDQGNKGQFLVPRYKPRTGDAVVFGHEKPEDLRSIRDKLLDAEKTTLRTWLAATSLPDWLKERTRYLHTWRAPGKLGALLTTWKKQRFDGDEEIVRKLEAWSKQDRHLLDWESSQRDRAQNHRREEYRLIANKIARVYSKIIIEVFDLRTFAKAKQPEDGLPSDGRRQRSNRMIASPSELRNVIRQAAVKTGAEVIEESAEYTTLTCALCGFTEKWDAAPAIIHTCGRCGEEWDQDENACRNLLGASGKAITSDGEALENGKGADPRGKLRRSEFRQETVVAE

SEQ ID NO: 325 - protein, 585 aa, unidentified; identical to SEQ ID NO: 19
  REGION 1..585: note = MG126-8 Effector; note = uncultivated organism

SEQ ID NO: 326 - RNA, 163 nt, unidentified
  source 1..163: mol_type = other RNA; note = uncultivated organism; MG119-3_IG1
attcttttattatgcgacttcgaacttgcaaagttaggaaaagttttcgtaaggaataactgaaaaaagttattatagtagccttttactcaaataattttaagcatagcaaactgttgatttacaaaataaatttaattacttttacactaaagtatttttt

SEQ ID NO: 327 - RNA, 163 nt, unidentified; identical to SEQ ID NO: 326
  source 1..163: mol_type = other RNA; note = uncultivated organism; MG119-3_IG2

SEQ ID NO: 328 - RNA, 205 nt, unidentified
  source 1..205: mol_type = other RNA; note = uncultivated organism; MG119-3_IG3
gctacctttgccacatacagagttctttaaaattttagatatgatttttatattccctttatgggaagggtgtgttggaacgccttagtttgaggtttgaatataaaaagtgaaacaaatggagatttgtaacaccttcaaacactcaaaaaattcatttattttttgatataaatgttggatttcaacgacttttactacatat

SEQ ID NO: 329 - RNA, 137 nt, unidentified
  source 1..137: mol_type = other RNA; note = uncultivated organism; MG119-4_IG1
aacttttgttttatacattaatatgggcaaaaatactaaattttacgaaatacacttaactttcattcgtctctaattacaatagcagactatttttcctttgaaaataactacctttgtcaatctttataaaaata

SEQ ID NO: 330 - RNA, 162 nt, unidentified
  source 1..162: mol_type = other RNA; note = uncultivated organism; MG119-4_IG2
atttctcgaaatagaatttacttgtggaatccctcatgcggttgcatgggggattttttatgggcgattttgagaatttgtgaaattctgaaaattatttgagttggatttgagacttacaattataattacaatatttgcggtggtaaaattaaaaagagt

SEQ ID NO: 331 - RNA, 107 nt, unidentified
  source 1..107: mol_type = other RNA; note = uncultivated organism; MG119-4_IG3
aaaagaaattttctaattggggctgctggaagtccttaacctgagataggaaactatactcgggaagtacgtcaaaacattgaaattaataaacttagaaactaacg

SEQ ID NO: 332 - RNA, 202 nt, unidentified
  source 1..202: mol_type = other RNA; note = uncultivated organism; MG119-4_IG4
taacactctgatatcattaaatataagcctatttccttctcaaatcaattaatagggggctgatgatgattgagaggaaataggcctattatgtatttaaaagagaagtaatatagaggtgaatatagtgtattttacatttaaaggcctcacaataaatgattaaaaaatagattttgtaatcactacatttaagaagatt

SEQ ID NO: 333 - RNA, 108 nt, unidentified
  source 1..108: mol_type = other RNA; note = uncultivated organism; MG120-1_IG1
aaacaaatggttaggtcaagggtttaattaatatttagagaaaaatcatagaaacacaaccagcatctcaattgttatcaacaacccaattgtgtttcgttttttgct

SEQ ID NO: 334 - RNA, 206 nt, unidentified
  source 1..206: mol_type = other RNA; note = uncultivated organism; MG120-1_IG2
gaacttgaaataggcatcaaaccatttgttgaggttaatgcgagcggccagttggctttaaaaacaggctggcctctcgcaacctttaccgcaaaggacttacgacaaaaaatcttttgttttggaaagtgtttacaggttgggaaacatagcctctcgcaacttttatcctaaacattgataataattagacttataacaatgcc

SEQ ID NO: 335 - RNA, 219 nt, unidentified
  source 1..219: mol_type = other RNA; note = uncultivated organism; MG120-1_IG3
acgatcttcgtctttatgataacggaacccgccaatcgcacctttcgaaatttatgaataaaatcgggggcggacttgactgggctgactttttctgataaaattcagccaacaaatggcagggtcgagttttgaatctcaggtaccaattatagattgtgtctataaattggatagttttagacaaatggctagatcgagtttaggaggtttgtgttt

SEQ ID NO: 336 - RNA, 489 nt, unidentified
  source 1..489: mol_type = other RNA; note = uncultivated organism; MG119-1_IG1
attcatcgttgtcggacgatcaaagattgttcgaattttcggagaaaatatgccgcggtgggtttaatgaaacttaacttttataaccattcaacttaatcataatctaatcaaatgatcaaattgattaagaaaatgattagattgagtcggttagataacggaggtgaagaaagtgtggatcaaacgtgagacttatgaacatcttcggatctgcgaggaaatgaaaacgaagcttgaagatgaagtcaagcggttatccgaattgatttcttcttagaatccggattgcaaagtaggcccatggtgtaaagattgcgcacatattggaagagattcatctgtggtgcgttattatacccctagcattgagtttggttgtgcaaagacagtagctggagaagttatttactgcaaaaaacacatccacgatatgtgcccggaatttgaaaagtcctcttcagattcatatttttcgttattgtacaa

SEQ ID NO: 337 - RNA, 1259 nt, unidentified
  source 1..1259: mol_type = other RNA; note = uncultivated organism; MG119-1_IG2
ttacattacgattgcggtggcggaatagacgggctgcgattgattcgcagttcggtaaaaccagacgggaagccaaggtggactgaatggtaacagtagacgctagacgggtagcaagatggaaacgaccgtcccgtagaccgatggtctcgccaaaatccacggggttcttcttgtatgtgaggtgcaaatcctcacccgtaatttgatttaggtgattcgtatgagaaaattgatttggtatttaagatcttgcttttgtaggcatgattgggttgtagaagagcaaccattcagagaatactttgaagggacgcttactgagcaaaccactgtagtgtctatgagatgcacaaaatgtggttggcatcagtcatataagaagtttaagttttgatttcatgcggcagtagctcagatggtagaggacgccaacaacggtgatcggtcgccggttcgagtccggcctgccgaatcaacataatgaggtaaaactatggagaaatacggagaaatatatttaccaaaacatcatagagccaaaaaaaatggatatgtagatgagcatattgtaatcgcagaacaaaaaatagggagatagttaaaaaaggatgaggtggttcatcataaagacaaagataaatccaataataaccctgaaaatctgatggtttttacaacaatggctgcacatacggcatttcatagaggaggaaaattgataagccttcccgatgggacttatgattgcgaacggctcccaataagatattgtgaaatatgtgggaagcaacttaaaacaaaacgagcaaaaagatgtcaaaaatgcgaccatataagacaaagaaaatgcgattggccaacaaaagagtagctaataaaagatttgaatgactttaaaaccaatgttgctctcgcagaaaaatatggggtaagtgataaaaccattgcaaaatggaagaaaaaaatactatcacaatctgaatctgaaaaataagctataagatacattcataatctatattcccggtaactgtttccagttacctataatatgctatcgtagctcagctggcagagcaatcgttttgtaaacgataggtcgtgggttcgagtcccaccgatagctccatccgaaaaagggtcagtagtagggagtgcaaaccagtgaccctgagacccgcaaggggcaacacccaagcgtgtaaggcgtagtacgctgttaggtattcaatcaagatgagggagtgaggcaagtc

SEQ ID NO: 338 - RNA, 173 nt, unidentified
  source 1..173: mol_type = other RNA; note = uncultivated organism; MG119-1_IG3
gtaggttcgcaccaaatatagggcgacttggcgtccttaaaccgagaagtgcatcagaaggtttacatagatgtaacactcggtaaggtaaatccgtaaggataatccaccccggcgaaatgccggccactaaaatgcataaagcgcaagttaatattttgtacggttttgcc

SEQ ID NO: 339 - RNA, 728 nt, unidentified
  source 1..728: mol_type = other RNA; note = uncultivated organism; MG119-1_IG4
aaataggagagagcatctaaattttccaaaacagaaaaataactgcctccttagctcaatcggcagagcgccagactcataatctgtgaggttccgggttcgatcccccgagggggcaccagtgataccaaaggacggaacaggcagccgtcgcccaatgttaggagatgcgggagtgtcaccccgcttggtatctcaacaactgaatagcccagacgggcaaaacatttccgagtgatccgtcatcgctcggagacatcgaggtgtagcgcagtggcagcgctcctgctttgggagcaggatgccgcgggttcgaatcccgccacctcgaccaaacgccgccgtggtggaatggcagacacaagcgacttaaaatctcttgctctcagagcgtatgggttcgagtcccatcggcggcaccatttgcttagttggcggaatcggcagacgcaccgcactcaaaatgcggcggagaaatccatgagggttcaaatccctcactaagcaccagtaaccctgttcgctatgctcacgtttgcgcagtttggttcgcgaccgtacttcaatctctgtgagaagtcgaggcggcggccgacgctgttcttcggacgcagggtctgtaataggggtgtagccaagcggcaaggcaaaggactttgactccttgattcgccggttcgatcccggccacccctgccaatttcattttaaagttcaggagggttttt

SEQ ID NO: 340 - RNA, 1074 nt, unidentified
  source 1..1074: mol_type = other RNA; note = uncultivated organism; MG119-1_IG5
aaattctatcgctgccattcgaaaatctgaaacagtgcttcatctatgttcgcgtaccacatcgatgttaaaagtaaaaaaggcacgcaccaatcggctaaattggattttaattctaattcgacgcgaaaatcgaataagaatctataaaaaattgctgtaaagctaaaagatcagaagctgaaagctcaaaactcaatgctcaaagtttatttgatcaaagctcaaagatctaaaagtcaaagagtacagctttacaaaatccaaggtctcaggtttcgggcaagcactttgctatcgggttctcctcttggctgaatggtagcgatttgaatataacttcacgtacacaagaaaaggagaaatcctgtttttgattcacaactgcgtgaagccacgcggctttgccgcgtatatcatctaatccagctgcatgaggcggacagatgaatgtcaacaaacgtaggtgataaaatgtacggcgcattaacctcagaagaagaaattgaagctcgcatgttgtttcgaggagctaaaaatccgctaatgcagattgagatcatctgcgatctatactaggttgatctgaacacagtctgcaaagctctcaaattagagtgcgaccccgagcttctgaaaaagaatttaagggcaactcattatgatgaaaatccgacagtatatgcaaagattgtattaaactgggggacaatcagacaattcgctttacaaacggggatttgtgaacaatccgtccgtaaagcattgcgaagacgtaatatggcagactctaaaattgggaaaagaattttagaagaactgcaaaattaggaggatacaacccatgccaaaagcgccggtttgcagtcgctgcggtaagcaatctgcgaaaactcactttgaaaacaacgataaaagtagggttttatcatatgcggcgtaaaacccctgtcttcagacatggggagtatgtcaatgtgatattccaagacctttttgtgaggtgttccaagtaaagggttataaaaatctttatcgatatcttatcgatatggtagataagaggtaatatct

SEQ ID NO: 341 - RNA, 259 nt, unidentified
  source 1..259: mol_type = other RNA; note = uncultivated organism; MG119-2_IG1
ttcgtcttcttttttctatttattaaaaagattagaatttatgttataaattacattgaactgtttatactaatattgttgcaaagttgatttgcacaaagtatactatgcttagttacacatcattagatgtgagggcaattttatgtcctaatattgagggtattaggtaacaacactttgttttaatagaacagtgtctaataaccactcgataagaccatataaaaggtataaatagtgaatttgaggttttata

SEQ ID NO: 342 - RNA, 464 nt, unidentified
  source 1..464: mol_type = other RNA; note = uncultivated organism; MG119-2_IG2
tatacatattttataatatgtaagggcgattttacgtcctaatggtgaggatgttgacgtaaaaacattttgtttgaaatatatcaatgtgttaatgtcactcgctaaaaccatacaaatgtgtaaaataataatttgaggttttatacctacttaattttgtatggttataaaaccaggaatatcaccttttaagaagtcttcttgttttacacctactagaatttgcatggttgtaaaacgataaaaaaataataagtcaagttcaagcggttttacacctaccaaaatttgtatggttgtaaaactgaatatttaactctcgacttatatacaagttgtacacatactatataccgattaaaatacacaatttagaaataaaaatcatcaaaatatagataaaaagaggtcaaaaagacctctttttttattttagaatataaatttaagtcttcagtata

SEQ ID NO: 343 - RNA, 332 nt, unidentified
  source 1..332: mol_type = other RNA; note = uncultivated organism; MG119-5_IG1
aaaaatataataaaatattgtttacaaacaattaaaaataaattataataatattagattttgaaagagaggtgaaacgaaataaaatttcaaggggaggcttagatcggtgaatccgagtcctacgtgcgaatcgcatatggatgccccataacctgagatagtcagcgatgaaattgttgatacataaggatttacgtaaggtgcgaatctataaaaaatacgctgatgaaactcggtcaaggtattttccgaaaggaaaagtcctaactaatgttaggtgaaaaactcgatcacaaagcccgatcgagccacttacaactgaatacgga

SEQ ID NO: 344 - RNA, 169 nt, unidentified
  source 1..169: mol_type = other RNA; note = uncultivated organism; MG119-5_IG2
agaggtggtggcatattgtttggatttggtttttatagcaatgttgtaaaaggatttttacaactaattggattaatgtttgtttcatttattattggatttgttttaggtgcatggattttttgataaaagaatggtaattgatacataaattaggagggatattcaa

SEQ ID NO: 345 - RNA, 129 nt, unidentified
  source 1..129: mol_type = other RNA; note = uncultivated organism; MG119-5_IG3
taatccacctaaatatgatgaatattcaagtaatggtgtttatatttggggtactgaaaattctataaaaaaatacttagaaacttataaatgaaataaattaaaacaatattaataaggagcggttta

SEQ ID NO: 346 - RNA, 208 nt, unidentified
  source 1..208: mol_type = other RNA; note = uncultivated organism; MG90-3_IG1
ggcgatggaacctctgaggttgtgtggaagtgtgttggtctggttgcatgatctgtatctgtccaggctaccagagagttgtaagataaagctatctcttcctggtggatggtctaccgggtgcagtacctttggcgtggggttagagcacgtcactggtcagtctgaccccagccgcggggctaggtagcggcaaaggagccttacc

SEQ ID NO: 347 - RNA, 167 nt, unidentified
  source 1..167: mol_type = other RNA; note = uncultivated organism; MG90-3_IG2
ggaggttgcacggtacgaatcgtccctgctcagcctgcgaggaggcgtcgaagtcctcgacagcagctagcttgccgatatctgccgatgcgtccaaaatacctccaggctaagctatactggaggcattctggccgctcgtgatcgaaatttaaggagtagaggtt

SEQ ID NO: 348 - RNA, 206 nt, unidentified
  source 1..206: mol_type = other RNA; note = uncultivated organism; MG119-3_IG1_adapters
gaagtgccattccgcctgacctattcttttattatgcgacttcgaacttgcaaagttaggaaaagttttcgtaaggaataactgaaaaaagttattatagtagccttttactcaaataattttaagcatagcaaactgttgatttacaaaataaatttaattacttttacactaaagtattttttaggctaggtggaggctcagtg

SEQ ID NO: 349 - RNA, 248 nt, unidentified
  source 1..248: mol_type = other RNA; note = uncultivated organism; MG119-3_IG2_adapters
gaagtgccattccgcctgacctgctacctttgccacatacagagttctttaaaattttagatatgatttttatattccctttatgggaagggtgtgttggaacgccttagtttgaggtttgaatataaaaagtgaaacaaatggagatttgtaacaccttcaaacactcaaaaaattcatttattttttgatataaatgttggatttcaacgacttttactacatataggctaggtggaggctcagtg

SEQ ID NO: 350 - RNA, 180 nt, unidentified
  source 1..180: mol_type = other RNA; note = uncultivated organism; MG119-3_IG3_adapters
gaagtgccattccgcctgacctaacttttgttttatacattaatatgggcaaaaatactaaattttacgaaatacacttaactttcattcgtctctaattacaatagcagactatttttcctttgaaaataactacctttgtcaatctttataaaaataaggctaggtggaggctcagtg

SEQ ID NO: 351 - RNA, 205 nt, unidentified
  source 1..205: mol_type = other RNA; note = uncultivated organism; MG119-4_IG1_adapters
gaagtgccattccgcctgacctatttctcgaaatagaatttacttgtggaatccctcatgcggttgcatgggggattttttatgggcgattttgagaatttgtgaaattctgaaaattatttgagttggatttgagacttacaattataattacaatatttgcggtggtaaaattaaaaagagtaggctaggtggaggctcagtg

SEQ ID NO: 352 - RNA, 150 nt, unidentified
  source 1..150: mol_type = other RNA; note = uncultivated organism; MG119-4_IG2_adapters
gaagtgccattccgcctgacctaaaagaaattttctaattggggctgctggaagtccttaacctgagataggaaactatactcgggaagtacgtcaaaacattgaaattaataaacttagaaactaacgaggctaggtggaggctcagtg

SEQ ID NO: 353 - RNA, 307 nt, unidentified
  source 1..307: mol_type = other RNA; note = uncultivated organism; MG119-4_IG3_adapters
gaagtgccattccgcctgaccttataaagtcgtctgcgatgatgtgcgcatgggtgttaataatcgtgtctttttttacttattttagttataaggtaatatacttataagggttattattgctaacgcatgtggttgcttatcacatacgttagcaatcaatcattacatacgttagtaataatcaacgactaacgtatgtaataatctgcaagaaaactttgccatttcaaataaaattgtaattttgctattacagtccccattaataaaaaaggaggtcagtaggctaggtggaggctcagtg

SEQ ID NO: 354 - RNA, 245 nt, unidentified
  source 1..245: mol_type = other RNA; note = uncultivated organism; MG119-4_IG4_adapters
gaagtgccattccgcctgaccttaacactctgatatcattaaatataagcctatttccttctcaaatcaattaatagggggctgatgatgattgagaggaaataggcctattatgtatttaaaagagaagtaatatagaggtgaatatagtgtattttacatttaaaggcctcacaataaatgattaaaaaatagattttgtaatcactacatttaagaagattaggctaggtggaggctcagtg

SEQ ID NO: 355 - RNA, 151 nt, unidentified
  source 1..151: mol_type = other RNA; note = uncultivated organism; MG120-1_IG1_adapters
gaagtgccattccgcctgacctaaacaaatggttaggtcaagggtttaattaatatttagagaaaaatcatagaaacacaaccagcatctcaattgttatcaacaacccaattgtgtttcgttttttgctaggctaggtggaggctcagtg

SEQ ID NO: 356 - RNA, 249 nt, unidentified
  source 1..249: mol_type = other RNA; note = uncultivated organism; MG120-1_IG2_adapters
gaagtgccattccgcctgacctgaacttgaaataggcatcaaaccatttgttgaggttaatgcgagcggccagttggctttaaaaacaggctggcctctcgcaacctttaccgcaaaggacttacgacaaaaaatcttttgttttggaaagtgtttacaggttgggaaacatagcctctcgcaacttttatcctaaacattgataataattagacttataacaatgccaggctaggtggaggctcagtg

SEQ ID NO: 357 - RNA, 262 nt, unidentified
  source 1..262: mol_type = other RNA; note = uncultivated organism; MG120-1_IG3_adapters
gaagtgccattccgcctgacctacgatcttcgtctttatgataacggaacccgccaatcgcacctttcgaaatttatgaataaaatcgggggcggacttgactgggctgactttttctgataaaattcagccaacaaatggcagggtcgagttttgaatctcaggtaccaattatagattgtgtctataaattggatagttttagacaaatggctagatcgagtttaggaggtttgtgtttaggctaggtggaggctcagtg

SEQ ID NO: 358 - RNA, 532 nt, unidentified
  source 1..532: mol_type = other RNA; note = uncultivated organism; MG119-1_IG1_adapters
gaagtgccattccgcctgacctattcatcgttgtcggacgatcaaagattgttcgaattttcggagaaaatatgccgcggtgggtttaatgaaacttaacttttataaccattcaacttaatcataatctaatcaaatgatcaaattgattaagaaaatgattagattgagtcggttagataacggaggtgaagaaagtgtggatcaaacgtgagacttatgaacatcttcggatctgcgaggaaatgaaaacgaagcttgaagatgaagtcaagcggttatccgaattgatttcttcttagaatccggattgcaaagtaggcccatggtgtaaagattgcgcacatattggaagagattcatctgtggtgcgttattatacccctagcattgagtttggttgtgcaaagacagtagctggagaagttatttactgcaaaaaacacatccacgatatgtgcccggaatttgaaaagtcctcttcagattcatatttttcgttattgtacaaaggctaggtggaggctcagtg

SEQ ID NO: 359 - RNA, 1302 nt, unidentified
  source 1..1302: mol_type = other RNA; note = uncultivated organism; MG119-1_IG2_adapters
gaagtgccattccgcctgacctttacattacgattgcggtggcggaatagacgggctgcgattgattcgcagttcggtaaaaccagacgggaagccaaggtggactgaatggtaacagtagacgctagacgggtagcaagatggaaacgaccgtcccgtagaccgatggtctcgccaaaatccacggggttcttcttgtatgtgaggtgcaaatcctcacccgtaatttgatttaggtgattcgtatgagaaaattgatttggtatttaagatcttgcttttgtaggcatgattgggttgtagaagagcaaccattcagagaatactttgaagggacgcttactgagcaaaccactgtagtgtctatgagatgcacaaaatgtggttggcatcagtcatataagaagtttaagttttgatttcatgcggcagtagctcagatggtagaggacgccaacaacggtgatcggtcgccggttcgagtccggcctgccgaatcaacataatgaggtaaaactatggagaaatacggagaaatatatttaccaaaacatcatagagccaaaaaaaatggatatgtagatgagcatattgtaatcgcagaacaaaaaatagggagatagttaaaaaaggatgaggtggttcatcataaagacaaagataaatccaataataaccctgaaaatctgatggtttttacaacaatggctgcacatacggcatttcatagaggaggaaaattgataagccttcccgatgggacttatgattgcgaacggctcccaataagatattgtgaaatatgtgggaagcaacttaaaacaaaacgagcaaaaagatgtcaaaaatgcgaccatataagacaaagaaaatgcgattggccaacaaaagagtagctaataaaagatttgaatgactttaaaaccaatgttgctctcgcagaaaaatatggggtaagtgataaaaccattgcaaaatggaagaaaaaaatactatcacaatctgaatctgaaaaataagctataagatacattcataatctatattcccggtaactgtttccagttacctataatatgctatcgtagctcagctggcagagcaatcgttttgtaaacgataggtcgtgggttcgagtcccaccgatagctccatccgaaaaagggtcagtagtagggagtgcaaaccagtgaccctgagacccgcaaggggcaacacccaagcgtgtaaggcgtagtacgctgttaggtattcaatcaagatgagggagtgaggcaagtcaggctaggtggaggctcagtg

SEQ ID NO: 360 - RNA, 216 nt, unidentified
  source 1..216: mol_type = other RNA; note = uncultivated organism; MG119-1_IG3_adapters
gaagtgccattccgcctgacctgtaggttcgcaccaaatatagggcgacttggcgtccttaaaccgagaagtgcatcagaaggtttacatagatgtaacactcggtaaggtaaatccgtaaggataatccaccccggcgaaatgccggccactaaaatgcataaagcgcaagttaatattttgtacggttttgccaggctaggtggaggctcagtg

SEQ ID NO: 361 - RNA, 771 nt, unidentified
  source 1..771: mol_type = other RNA; note = uncultivated organism; MG119-1_IG4_adapters
gaagtgccattccgcctgacctaaataggagagagcatctaaattttccaaaacagaaaaataactgcctccttagctcaatcggcagagcgccagactcataatctgtgaggttccgggttcgatcccccgagggggcaccagtgataccaaaggacggaacaggcagccgtcgcccaatgttaggagatgcgggagtgtcaccccgcttggtatctcaacaactgaatagcccagacgggcaaaacatttccgagtgatccgtcatcgctcggagacatcgaggtgtagcgcagtggcagcgctcctgctttgggagcaggatgccgcgggttcgaatcccgccacctcgaccaaacgccgccgtggtggaatggcagacacaagcgacttaaaatctcttgctctcagagcgtatgggttcgagtcccatcggcggcaccatttgcttagttggcggaatcggcagacgcaccgcactcaaaatgcggcggagaaatccatgagggttcaaatccctcactaagcaccagtaaccctgttcgctatgctcacgtttgcgcagtttggttcgcgaccgtacttcaatctctgtgagaagtcgaggcggcggccgacgctgttcttcggacgcagggtctgtaataggggtgtagccaagcggcaaggcaaaggactttgactccttgattcgccggttcgatcccggccacccctgccaatttcattttaaagttcaggagggtttttaggctaggtggaggctcagtg

SEQ ID NO: 362 - RNA, 1117 nt, unidentified
  source 1..1117: mol_type = other RNA; note = uncultivated organism; MG119-1_IG5_adapters
gaagtgccattccgcctgacctaaattctatcgctgccattcgaaaatctgaaacagtgcttcatctatgttcgcgtaccacatcgatgttaaaagtaaaaaaggcacgcaccaatcggctaaattggattttaattctaattcgacgcgaaaatcgaataagaatctataaaaaattgctgtaaagctaaaagatcagaagctgaaagctcaaaactcaatgctcaaagtttatttgatcaaagctcaaagatctaaaagtcaaagagtacagctttacaaaatccaaggtctcaggtttcgggcaagcactttgctatcgggttctcctcttggctgaatggtagcgatttgaatataacttcacgtacacaagaaaaggagaaatcctgtttttgattcacaactgcgtgaagccacgcggctttgccgcgtatatcatctaatccagctgcatgaggcggacagatgaatgtcaacaaacgtaggtgataaaatgtacggcgcattaacctcagaagaagaaattgaagctcgcatgttgtttcgaggagctaaaaatccgctaatgcagattgagatcatctgcgatctatactaggttgatctgaacacagtctgcaaagctctcaaattagagtgcgaccccgagcttctgaaaaagaatttaagggcaactcattatgatgaaaatccgacagtatatgcaaagattgtattaaactgggggacaatcagacaattcgctttacaaacggggatttgtgaacaatccgtccgtaaagcattgcgaagacgtaatatggcagactctaaaattgggaaaagaattttagaagaactgcaaaattaggaggatacaacccatgccaaaagcgccggtttgcagtcgctgcggtaagcaatctgcgaaaactcactttgaaaacaacgataaaagtagggttttatcatatgcggcgtaaaacccctgtcttcagacatggggagtatgtcaatgtgatattccaagacctttttgtgaggtgttccaagtaaagggttataaaaatctttatcgatatcttatcgatatggtagataagaggtaatatctaggctaggtggaggctcagtg

SEQ ID NO: 363 - RNA, 302 nt, unidentified
  source 1..302: mol_type = other RNA; note = uncultivated organism; MG119-2_IG1_adapters
gaagtgccattccgcctgacctttcgtcttcttttttctatttattaaaaagattagaatttatgttataaattacattgaactgtttatactaatattgttgcaaagttgatttgcacaaagtatactatgcttagttacacatcattagatgtgagggcaattttatgtcctaatattgagggtattaggtaacaacactttgttttaatagaacagtgtctaataaccactcgataagaccatataaaaggtataaatagtgaatttgaggttttataaggctaggtggaggctcagtg

SEQ ID NO: 364 - RNA, 507 nt, unidentified
  source 1..507: mol_type = other RNA; note = uncultivated organism; MG119-2_IG2_adapters
gaagtgccattccgcctgaccttatacatattttataatatgtaagggcgattttacgtcctaatggtgaggatgttgacgtaaaaacattttgtttgaaatatatcaatgtgttaatgtcactcgctaaaaccatacaaatgtgtaaaataataatttgaggttttatacctacttaattttgtatggttataaaaccaggaatatcaccttttaagaagtcttcttgttttacacctactagaatttgcatggttgtaaaacgataaaaaaataataagtcaagttcaagcggttttacacctaccaaaatttgtatggttgtaaaactgaatatttaactctcgacttatatacaagttgtacacatactatataccgattaaaatacacaatttagaaataaaaatcatcaaaatatagataaaaagaggtcaaaaagacctctttttttattttagaatataaatttaagtcttcagtataaggctaggtggaggctcagtg

SEQ ID NO: 365 - RNA, 375 nt, unidentified
  source 1..375: mol_type = other RNA; note = uncultivated organism; MG119-5_IG1_adapters
gaagtgccattccgcctgacctaaaaatataataaaatattgtttacaaacaattaaaaataaattataataatattagattttgaaagagaggtgaaacgaaataaaatttcaaggggaggcttagatcggtgaatccgagtcctacgtgcgaatcgcatatggatgccccataacctgagatagtcagcgatgaaattgttgatacataaggatttacgtaaggtgcgaatctataaaaaatacgctgatgaaactcggtcaaggtattttccgaaaggaaaagtcctaactaatgttaggtgaaaaactcgatcacaaagcccgatcgagccacttacaactgaatacggaaggctaggtggaggctcagtg

SEQ ID NO: 366 - RNA, 212 nt, unidentified
  source 1..212: mol_type = other RNA; note = uncultivated organism; MG119-5_IG2_adapters
gaagtgccattccgcctgacctagaggtggtggcatattgtttggatttggtttttatagcaatgttgtaaaaggatttttacaactaattggattaatgtttgtttcatttattattggatttgttttaggtgcatggattttttgataaaagaatggtaattgatacataaattaggagggatattcaaaggctaggtggaggctcagtg

SEQ ID NO: 367 - RNA, 172 nt, unidentified
  source 1..172: mol_type = other RNA; note = uncultivated organism; MG119-5_IG3_adapters
gaagtgccattccgcctgaccttaatccacctaaatatgatgaatattcaagtaatggtgtttatatttggggtactgaaaattctataaaaaaatacttagaaacttataaatgaaataaattaaaacaatattaataaggagcggtttaaggctaggtggaggctcagtg

SEQ ID NO: 368 - RNA, 251 nt, unidentified
  source 1..251: mol_type = other RNA; note = uncultivated organism; MG90-3_IG1_adapters
gaagtgccattccgcctgacctggcgatggaacctctgaggttgtgtggaagtgtgttggtctggttgcatgatctgtatctgtccaggctaccagagagttgtaagataaagctatctcttcctggtggatggtctaccgggtgcagtacctttggcgtggggttagagcacgtcactggtcagtctgaccccagccgcggggctaggtagcggcaaaggagccttaccaggctaggtggaggctcagtg

SEQ ID NO: 369 - RNA, 210 nt, unidentified
  source 1..210: mol_type = other RNA; note = uncultivated organism; MG90-3_IG2_adapters
gaagtgccattccgcctgacctggaggttgcacggtacgaatcgtccctgctcagcctgcgaggaggcgtcgaagtcctcgacagcagctagcttgccgatatctgccgatgcgtccaaaatacctccaggctaagctatactggaggcattctggccgctcgtgatcgaaatttaaggagtagaggttaggctaggtggaggctcagtg

SEQ ID NO: 370 - DNA, 121 bp, unidentified
  source 1..121: mol_type = other DNA; note = uncultivated organism; 119-3_5U40_31_F
taatacgactcactataggttgtaaaagcattaatttttaaatgaatatccaactggagatatcttgaaccttgcatccccggaagttgtaaaagcattaatttttaaatgaatatccaac

SEQ ID NO: 371 - DNA, 121 bp, unidentified
  source 1..121: mol_type = other DNA; note = uncultivated organism; 119-3_5U40_31_R
taatacgactcactataggttggatattcatttaaaaattaatgcttttacaactggagatatcttgaaccttgcatccccggaagttggatattcatttaaaaattaatgcttttacaac

SEQ ID NO: 372 - DNA, 121 bp, unidentified
  source 1..121: mol_type = other DNA; note = uncultivated organism; 119-4_5U40_31_F
taatacgactcactataggttggttttatcattgttttgaatggtatatccaactggagatatcttgaaccttgcatccccggaagttggttttatcattgttttgaatggtatatccaac

SEQ ID NO: 373 - DNA, 121 bp, unidentified
  source 1..121: mol_type = other DNA; note = uncultivated organism; 119-4_5U40_31_R
taatacgactcactataggttggatataccattcaaaacaatgataaaaccaactggagatatcttgaaccttgcatccccggaagttggatataccattcaaaacaatgataaaaccaac

SEQ ID NO: 374 - DNA, 127 bp, unidentified
  source 1..127: mol_type = other DNA; note = uncultivated organism; 120-1_5U40_37_F
taatacgactcactatagatagcaacgaatgaaaatacaaatggttagatcgagtggagatatcttgaaccttgcatccccggaagagagtatagcaacgaatgaaaatacaaatggttagatcgag

SEQ ID NO: 375 - DNA, 127 bp, unidentified
  source 1..127: mol_type = other DNA; note = uncultivated organism; 120-1_5U40_37_R
taatacgactcactatagctcgatctaaccatttgtattttcattcgttgctattggagatatcttgaaccttgcatccccggaagagagtctcgatctaaccatttgtattttcattcgttgctat

SEQ ID NO: 376 - DNA, 128 bp, unidentified
  source 1..128: mol_type = other DNA; note = uncultivated organism; 118-1_5U40_38_R
taatacgactcactataggtaacgactgataaaaattgctcactacggtgagactggagatatcttgaaccttgcatccccggaagagagtcgtaacgactgataaaaattgctcactacggtgagac

SEQ ID NO: 377 - DNA, 114 bp, unidentified
  source 1..114: mol_type = other DNA; note = uncultivated organism; 119-1_5U67_32_F
taatacgactcactataggacgcaacttgcgcgtatgcacaagtggaagtgtcgaggcttgcgacgtggtggctcacggaaggacgcaacttgcgcgtatgcacaagtggaagt

SEQ ID NO: 378 - DNA, 114 bp, unidentified
  source 1..114: mol_type = other DNA; note = uncultivated organism; 119-1_5U67_32_R
taatacgactcactatagacttccacttgtgcatacgcgcaagttgcgtcgtcgaggcttgcgacgtggtggctcacggaagacttccacttgtgcatacgcgcaagttgcgtc

SEQ ID NO: 379 - DNA, 102 bp, unidentified
  source 1..102: mol_type = other DNA; note = uncultivated organism; 119-2_5U40_28_F
taatacgactcactatagcctacttaattttgtatggttataaaactggagatatcttgaaccttgcatccccgcctacttaattttgtatggttataaaac

SEQ ID NO: 380 - DNA, 102 bp, unidentified
  source 1..102: mol_type = other DNA; note = uncultivated organism; 119-2_5U40_28_R
taatacgactcactataggttttataaccatacaaaattaagtaggtggagatatcttgaaccttgcatccccggttttataaccatacaaaattaagtagg

SEQ ID NO: 381 - DNA, 111 bp, unidentified
  source 1..111: mol_type = other DNA; note = uncultivated organism; 119-5_5U40_31_F
taatacgactcactataggttgcaatacgtatgcgtatgtaagtggtaatggagatatcttgaaccttgcatccccggaagttgcaatacgtatgcgtatgtaagtggtaa

SEQ ID NO: 382 - DNA, 111 bp, unidentified
  source 1..111: mol_type = other DNA; note = uncultivated organism; 119-5_5U40_31_R
taatacgactcactatagttaccacttacatacgcatacgtattgcaactggagatatcttgaaccttgcatccccggaattaccacttacatacgcatacgtattgcaac

SEQ ID NO: 383 - DNA, 129 bp, unidentified
  source 1..129: mol_type = other DNA; note = uncultivated organism; 90-3_5U67_37_F
taatacgactcactatagggtgcagtacctttggcgtggggttagagcacgtcacgtcgaggcttgcgacgtggtggctcacggaagacgaaggtgcagtacctttggcgtggggttagagcacgtcac

SEQ ID NO: 384 - DNA, 129 bp, unidentified
  source 1..129: mol_type = other DNA; note = uncultivated organism; 90-3_5U67_37_R
taatacgactcactataggtgacgtgctctaaccccacgccaaaggtactgcaccgtcgaggcttgcgacgtggtggctcacggaagacgaagtgacgtgctctaaccccacgccaaaggtactgcacc

SEQ ID NO: 385 - DNA, 164 bp, unidentified
  source 1..164: mol_type = other DNA; note = uncultivated organism; 119-3_5U40_31_F_adapters
gaagtgccattccgcctgaccttaatacgactcactataggttgtaaaagcattaatttttaaatgaatatccaactggagatatcttgaaccttgcatccccggaagttgtaaaagcattaatttttaaatgaatatccaacaggctaggtggaggctcagtg

SEQ ID NO: 386 - DNA, 164 bp, unidentified
  source 1..164: mol_type = other DNA; note = uncultivated organism; 119-3_5U40_31_R_adapters
gaagtgccattccgcctgaccttaatacgactcactataggttggatattcatttaaaaattaatgcttttacaactggagatatcttgaaccttgcatccccggaagttggatattcatttaaaaattaatgcttttacaacaggctaggtggaggctcagtg

SEQ ID NO: 387 - DNA, 164 bp, unidentified
  source 1..164: mol_type = other DNA; note = uncultivated organism; 119-4_5U40_31_F_adapters
gaagtgccattccgcctgaccttaatacgactcactataggttggttttatcattgttttgaatggtatatccaactggagatatcttgaaccttgcatccccggaagttggttttatcattgttttgaatggtatatccaacaggctaggtggaggctcagtg

SEQ ID NO: 388 - DNA, 164 bp, unidentified
  source 1..164: mol_type = other DNA; note = uncultivated organism; 119-4_5U40_31_R_adapters
gaagtgccattccgcctgaccttaatacgactcactataggttggatataccattcaaaacaatgataaaaccaactggagatatcttgaaccttgcatccccggaagttggatataccattcaaaacaatgataaaaccaacaggctaggtggaggctcagtg

SEQ ID NO: 389 - DNA, 170 bp, unidentified
  source 1..170: mol_type = other DNA; note = uncultivated organism; 120-1_5U40_37_F_adapters
gaagtgccattccgcctgaccttaatacgactcactatagatagcaacgaatgaaaatacaaatggttagatcgagtggagatatcttgaaccttgcatccccggaagagagtatagcaacgaatgaaaatacaaatggttagatcgagaggctaggtggaggctcagtg

SEQ ID NO: 390 - DNA, 170 bp, unidentified
  source 1..170: mol_type = other DNA; note = uncultivated organism; 120-1_5U40_37_R_adapters
gaagtgccattccgcctgaccttaatacgactcactatagctcgatctaaccatttgtattttcattcgttgctattggagatatcttgaaccttgcatccccggaagagagtctcgatctaaccatttgtattttcattcgttgctataggctaggtggaggctcagtg

SEQ ID NO: 391 - DNA, 171 bp, unidentified
  source 1..171: mol_type = other DNA; note = uncultivated organism; 118-1_5U40_38_R_adapters
gaagtgccattccgcctgaccttaatacgactcactataggtaacgactgataaaaattgctcactacggtgagactggagatatcttgaaccttgcatccccggaagagagtcgtaacgactgataaaaattgctcactacggtgagacaggctaggtggaggctcagtg

SEQ ID NO: 392 - DNA, 157 bp, unidentified
  source 1..157: mol_type = other DNA; note = uncultivated organism; 119-1_5U67_32_F_adapters
gaagtgccattccgcctgaccttaatacgactcactataggacgcaacttgcgcgtatgcacaagtggaagtgtcgaggcttgcgacgtggtggctcacggaaggacgcaacttgcgcgtatgcacaagtggaagtaggctaggtggaggctcagtg

SEQ ID NO: 393 - DNA, 157 bp, unidentified
  source 1..157: mol_type = other DNA; note = uncultivated organism; 119-1_5U67_32_R_adapters
gaagtgccattccgcctgaccttaatacgactcactatagacttccacttgtgcatacgcgcaagttgcgtcgtcgaggcttgcgacgtggtggctcacggaagacttccacttgtgcatacgcgcaagttgcgtcaggctaggtggaggctcagtg

SEQ ID NO: 394 - DNA, 145 bp, unidentified
  source 1..145: mol_type = other DNA; note = uncultivated organism; 119-2_5U40_28_F_adapters
gaagtgccattccgcctgaccttaatacgactcactatagcctacttaattttgtatggttataaaactggagatatcttgaaccttgcatccccgcctacttaattttgtatggttataaaacaggctaggtggaggctcagtg

SEQ ID NO: 395 - DNA, 145 bp, unidentified
  source 1..145: mol_type = other DNA; note = uncultivated organism; 119-2_5U40_28_R_adapters
gaagtgccattccgcctgaccttaatacgactcactataggttttataaccatacaaaattaagtaggtggagatatcttgaaccttgcatccccggttttataaccatacaaaattaagtaggaggctaggtggaggctcagtg

SEQ ID NO: 396 - DNA, 154 bp, unidentified
  source 1..154: mol_type = other DNA; note = uncultivated organism; 119-5_5U40_31_F_adapters
gaagtgccattccgcctgaccttaatacgactcactataggttgcaatacgtatgcgtatgtaagtggtaatggagatatcttgaaccttgcatccccggaagttgcaatacgtatgcgtatgtaagtggtaaaggctaggtggaggctcagtg

SEQ ID NO: 397 - DNA, 154 bp, unidentified
  source 1..154: mol_type = other DNA; note = uncultivated organism; 119-5_5U40_31_R_adapters
gaagtgccattccgcctgaccttaatacgactcactatagttaccacttacatacgcatacgtattgcaactggagatatcttgaaccttgcatccccggaattaccacttacatacgcatacgtattgcaacaggctaggtggaggctcagtg

SEQ ID NO: 398 - DNA, 172 bp, unidentified
  source 1..172: mol_type = other DNA; note = uncultivated organism; 90-3_5U67_37_F_adapters
gaagtgccattccgcctgaccttaatacgactcactatagggtgcagtacctttggcgtggggttagagcacgtcacgtcgaggcttgcgacgtggtggctcacggaagacgaaggtgcagtacctttggcgtggggttagagcacgtcacaggctaggtggaggctcagtg

SEQ ID NO: 399 - DNA, 172 bp, unidentified
  source 1..172: mol_type = other DNA; note = uncultivated organism; 90-3_5U67_37_R_adapters
gaagtgccattccgcctgaccttaatacgactcactataggtgacgtgctctaaccccacgccaaaggtactgcaccgtcgaggcttgcgacgtggtggctcacggaagacgaagtgacgtgctctaaccccacgccaaaggtactgcaccaggctaggtggaggctcagtg

SEQ ID NO: 400 - RNA, 61 nt, unidentified
  source 1..61: mol_type = other RNA; note = uncultivated organism; MG118-1_U40_18nt_target
taatacgactcactatagtaaaaattgctcactacggtgagactggagatatcttgaacct

SEQ ID NO: 401 - RNA, 61 nt, unidentified
  source 1..61: mol_type = other RNA; note = uncultivated organism; MG118-1_U67_18nt_target
taatacgactcactatagtaaaaattgctcactacggtgagacgtcgaggcttgcgacgtg

SEQ ID NO: 402 - RNA, 169 nt, unidentified
  source 1..169: mol_type = other RNA; note = uncultivated organism; 90-3_sgRNA_10bp_RAR_U40_24_target
taatacgactcactatagaggttgtgtggaagtgtgttggtctggttgcatgatctgtatctgtccaggctaccagagagttgtaagataaagctatctcttcctggtggatggtctaccgggtgcagtacgaaagtactgcacctggagatatcttgaaccttgcatc

SEQ ID NO: 403 - RNA, 181 nt, unidentified
  source 1..181: mol_type = other RNA; note = uncultivated organism; 90-3_sgRNA_16bp_RAR_U40_24_target
taatacgactcactatagaggttgtgtggaagtgtgttggtctggttgcatgatctgtatctgtccaggctaccagagagttgtaagataaagctatctcttcctggtggatggtctaccgggtgcagtacctttgggaaaccaaaggtactgcacctggagatatcttgaaccttgcatc

SEQ ID NO: 404 - RNA, 141 nt, unidentified
  source 1..141: mol_type = other RNA; note = uncultivated organism; 119-1_sgRNA_10bp_RAR_U40_24_target
taatacgactcactatagtttacatagatgtaacactcggtaaggtaaatccgtaaggataatccaccccggcgaaatgccggccactaaaatgcatgaaaatgcacaagtggaagttggagatatcttgaaccttgcatc

SEQ ID NO: 405 - RNA, 156 nt, unidentified
  source 1..156: mol_type = other RNA; note = uncultivated organism; 119-1_sgRNA_15bp_RAR_U40_24_target
taatacgactcactatagtttacatagatgtaacactcggtaaggtaaatccgtaaggataatccaccccggcgaaatgccggccactaaaatgcataaagcgcgaaattgcgcgtatgcacaagtggaagttggagatatcttgaaccttgcatc

SEQ ID NO: 406 - RNA, 160 nt, unidentified
  source 1..160: mol_type = other RNA; note = uncultivated organism; 119-2_sgRNA_10bp_RAR_U40_24_target
taatacgactcactatagtgacgtaaaaacattttgtttgaaatatatcaatgtgttaatgtcactcgctaaaaccatacaaatgtgtaaaataataatttgaggttttatacctacttaatgaaaattaagtaggtggagatatcttgaaccttgcatc

SEQ ID NO: 407 - RNA, 172 nt, unidentified
  source 1..172: mol_type = other RNA; note = uncultivated organism; 119-2_sgRNA_16bp_RAR_U40_24_target
taatacgactcactatagtgacgtaaaaacattttgtttgaaatatatcaatgtgttaatgtcactcgctaaaaccatacaaatgtgtaaaataataatttgaggttttatacctacttaattttgtagaaatacaaaattaagtaggtggagatatcttgaaccttgcatc

SEQ ID NO: 408 - RNA, 131 nt, unidentified
  source 1..131: mol_type = other RNA; note = uncultivated organism; 119-5_sgRNA_9bp_RAR_U40_24_target
taatacgactcactatagttttccgaaaggaaaagtcctaactaatgttaggtgaaaaactcgatcacaaagcccgatcgagccacttacagaaatgtaagtggtaatggagatatcttgaaccttgcatc

SEQ ID NO: 409 - RNA, 151 nt, unidentified
  source 1..151: mol_type = other RNA; note = uncultivated organism; 119-5_sgRNA_16bp_RAR_U40_24_target
taatacgactcactatagttttccgaaaggaaaagtcctaactaatgttaggtgaaaaactcgatcacaaagcccgatcgagccacttacaactgaatacggaaacgtatgcgtatgtaagtggtaatggagatatcttgaaccttgcatc

SEQ ID NO: 410 - RNA, 43 nt, unidentified
  source 1..43: mol_type = other RNA; note = uncultivated organism; MG118-1_U40_18nt
taaaaattgctcactacggtgagactggagatatcttgaacct

SEQ ID NO: 411 - RNA, 43 nt, unidentified
  source 1..43: mol_type = other RNA; note = uncultivated organism; MG118-1_U67_18nt
taaaaattgctcactacggtgagacgtcgaggcttgcgacgtg

SEQ ID NO: 412 - RNA, 151 nt, unidentified
  source 1..151: mol_type = other RNA; note = uncultivated organism, 90-3_sgRNA_10bp_RAR_U40_24
aggttgtgtggaagtgtgttggtctggttgcatgatctgtatctgtccaggctaccagagagttgtaagataaagctatctcttcctggtggatggtctaccgggtgcagtacgaaagtactgcacctggagatatcttgaaccttgcatc

SEQ ID NO: 413 - RNA, 163 nt, unidentified
  source 1..163: mol_type = other RNA; note = uncultivated organism; 90-3_sgRNA_16bp_RAR_U40_24
aggttgtgtggaagtgtgttggtctggttgcatgatctgtatctgtccaggctaccagagagttgtaagataaagctatctcttcctggtggatggtctaccgggtgcagtacctttgggaaaccaaaggtactgcacctggagatatcttgaaccttgcatc

SEQ ID NO: 414 - RNA, 123 nt, unidentified
  source 1..123: mol_type = other RNA; note = uncultivated organism; 119-1_sgRNA_10bp_RAR_U40_24
tttacatagatgtaacactcggtaaggtaaatccgtaaggataatccaccccggcgaaatgccggccactaaaatgcatgaaaatgcacaagtggaagttggagatatcttgaaccttgcatc

SEQ ID NO: 415 - RNA, 138 nt, unidentified
  source 1..138: mol_type = other RNA; note = uncultivated organism; 119-1_sgRNA_15bp_RAR_U40_24
tttacatagatgtaacactcggtaaggtaaatccgtaaggataatccaccccggcgaaatgccggccactaaaatgcataaagcgcgaaattgcgcgtatgcacaagtggaagttggagatatcttgaaccttgcatc

SEQ ID NO: 416 - RNA, 142 nt, unidentified
  source 1..142: mol_type = other RNA; note = uncultivated organism; 119-2_sgRNA_10bp_RAR_U40_24
tgacgtaaaaacattttgtttgaaatatatcaatgtgttaatgtcactcgctaaaaccatacaaatgtgtaaaataataatttgaggttttatacctacttaatgaaaattaagtaggtggagatatcttgaaccttgcatc

SEQ ID NO: 417 - RNA, 154 nt, unidentified
  source 1..154: mol_type = other RNA; note = uncultivated organism; 119-2_sgRNA_16bp_RAR_U40_24
tgacgtaaaaacattttgtttgaaatatatcaatgtgttaatgtcactcgctaaaaccatacaaatgtgtaaaataataatttgaggttttatacctacttaattttgtagaaatacaaaattaagtaggtggagatatcttgaaccttgcatc

SEQ ID NO: 418 - RNA, 113 nt, unidentified
  source 1..113: mol_type = other RNA; note = uncultivated organism; 119-5_sgRNA_9bp_RAR_U40_24
ttttccgaaaggaaaagtcctaactaatgttaggtgaaaaactcgatcacaaagcccgatcgagccacttacagaaatgtaagtggtaatggagatatcttgaaccttgcatc

SEQ ID NO: 419 - RNA, 133 nt, unidentified
  source 1..133: mol_type = other RNA; note = uncultivated organism; 119-5_sgRNA_16bp_RAR_U40_24
ttttccgaaaggaaaagtcctaactaatgttaggtgaaaaactcgatcacaaagcccgatcgagccacttacaactgaatacggaaacgtatgcgtatgtaagtggtaatggagatatcttgaaccttgcatc

SEQ ID NO: 420 - protein, 464 aa, unidentified
  source 1..464: note = uncultivated organism; MG119-124 Effector
MKTTRKLKLTIIGDEETRKEQYKIIREEQYQQYKALNLCMTLLNTHNILNSYNTGAENKLNAQIDSIDKKIEQAKKELEKKGLKESKVSKLKETIEFLENDREKLKDEYLNSSKFRSDIDEKMKEMYIKDMYTVVQNQVNFRARDMMSLVTQRARKDFKNSLKNGMAKGERSLTNYKRDFPLMTRGERWLKFEYDKDSDDILINWIHGIKFKVLLGYKKNENSIELRHTLHKVINKEYKICDSSMQFDRNNNLILNLTLDIPDKQNNNYIEKRTLGVDLGIKYPAYVCLNDDTYIRSHIGESLELLKQREQFKDRRKRLQQQLKNVKGGKGRNKKLSALNKLSDNERNFARTYNHMISKRIVEFAKKNRCEFINLEKLTKDGFDNNILSNWSYYELQNMIEYKAKREGIKVRYIDPAYTSQKCSRCGYIDKENRQTQEKFKCLKCEFEINADHNAAINIARALD

SEQ ID NO: 421 - protein, 403 aa, unidentified
  source 1..403: note = uncultivated organism; MG119-125 Effector
MSITVRKLKLIIVNSDDKARNNQYKFIRDSQYAQYQALNLGMGYMMAGYLACNRDIKSEEFKEWQKGLKNSNPVFQGIEYGKGIDTLSAVTQKIKKDFSTSLKNGLEKGERSSINYKRNFPLMTRGRNLNFYEEDGDIFIKWVNKILFKVVFNKRKENTIELQHTLNKVINGEYKVSQSSLEFDRNNNLILNLTMDIPFKKQEEFVEGRVLGVDLGIAVPAYVCLSDDTYKRKPIGSYEDFFKVRKQFKERRERLQRQLKMVKGGKGRNKKLQALNSFREKERNYAKTYNHNISKMIIDFAKQHKCKQINIENLSGNSFDEKILSSWSYYELQQMITYKAERIGINVKKVEASYTSQTCSRCGHIDKENRQTQDRFKCIKCDFELNADHNAAINIARSNKYVK

SEQ ID NO: 422 - protein, 403 aa, unidentified
  source 1..403: note = uncultivated organism; MG119-126 Effector
MAITVRKLKLTIVNGDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSFPLMTRGRNLTFYEENGEIYIKWVNKIVFKVVFNARKENTLELQHTLNKVITGEYKLSQSSLEFDRNNNLILNLTMDIPFKQQEEFVDGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMIAYKAERIGIEVKKVNPAYTSQTCSRCGNVDKENRQTQEKFKCTKCEFELNADHNAAINISRSKKYVK

SEQ ID NO: 423 - protein, 530 aa, unidentified
  source 1..530: note = uncultivated organism; MG119-127 Effector
MSKKGKKDKKNKKIVLCRKLKLTILGDEQTRKEQFKLIRDDQYQQYKALNLCMDLYQANHALRKYEFKLEVDSRKELNDSIPKKEKLIEKILTLEKQIISLEEKGKLNEKDEEKLEQKKSSLNKKNDELSALEEKILKLKNNIKNSIEYQASLSEKMQEKYIDDIHTVVQNQVNLNYKDTLSLVTQKVKEDFKIALNNGLKEGKRKSTSYKRDFPLMTKGRELKFKYRDENSDDIIIKWVKGITFKVILSNKKNNDKELRHTLHKIINNEVKICDSSLQFDNKNNLILNLAMEMKVERGFTPIEGRVMGVDLGMKIPAIASFNNMNGFKKFGSIDDFLRVRKQIQARRRKLQIALTTVSGGHGRDKKIQALDRFSKSEKNFVNTYNHTISKKIIEYAKVNKCGVINLEFLSLADQEKDLFLTMSESKIKQSRFIKRNWSYFDLQTKIIDKANREGIKVLLVDPYHTSQTCSKCGHYETGQRTSQAHFKCKSCGYEANADFNASQNIAKSTQYIDKKEDSQFFKNKKSKAS

SEQ ID NO: 424 - protein, 493 aa, unidentified
  source 1..493: note = uncultivated organism; MG119-128 Effector
MGKMDIKEEKIFRASNKVKITIVEDDPDERNRKYKLIHDEMYKQYLALNMCMSLLATQNTLRMYNSGAENRLLSQIAKLDKKISEKQQIADKDVNTEKEQTRKQKAIEDIAKLEEEKNKLREEFDKKATNRTDIDVKFHEQYTSDLYQIIRNEFDFASSITAGLVARRAKDDFKNSFSMGYAKGERSLISYKRSFPLMIRGNEDACYDSNKRKWLDFYYDGDDIFIRWLDGFIFKAILGNNPNRNMEIKHTLHKIITGEYRVSQSSIEFDDKGHMYMNVSYRFKKNSKTEIIPGRTLGVDLGIATPAYVCLSDTTYVRSGIGSANDFFRIRQQMKSRRRSLYKSLSLVKGGKGRKKKLQAINSLRDKERNFAKTYNHQLSNKIVKFAKDNGCEFINLEHLTKNGFEDAILSSWSYYELQNMIEYKAEREGIKVRYVDPAYTSQTCSKCGHVDKENRPTQAKFKCINCGLELNADHNASINIARSDNFVNKKKK

SEQ ID NO: 425 - protein, 416 aa, unidentified
  source 1..416: note = uncultivated organism; MG119-129 Effector
MINVRKLKLVIRETDKELRSQKYKFIRDSQYAQYRALNVAMGYISSAYLKANKDIKSEEYKEAIKLLTNSNPIFNGIEVGTGIDTLSAVTRKARKDFSASLKNGLARGERNLTSYRRTYPLITRGRDLKFRYEGDDIVIKWVNKIEFTVITGSNNMKENTIELKHTLHKIINNEYKIKESSLMFDKNNNLILNLTFDIPDKEIHDIVPGRTLGVDLGIATPAYVCLSDETYIRSEFGSANDFFRVRQQMKDRRKKLYASLTVVKGGKGRNKKLKAADRLRDKERNFVKTYNHQLSHKIVKFAKDNKCEFINLEYLTKDGFENRILSSWSYYELQNMIEYKAEREGIKVRYVDPAYTSQTCSKCGHVDKENRPTQAKFKCVNCGLELNADHNASINIARSDNFVNKKKKKKEDKQEE

SEQ ID NO: 426 - protein, 403 aa, unidentified
  source 1..403: note = uncultivated organism; MG119-130 Effector
MAITVRKLKLTIVNEDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSYPLMTRGRNLTFYEENGDIYIKWVNKIVFKVVFNARKENTLELQHTLNKVITGEYKLSQSSLEFDRNNNLILNLTMDIPFKQQEEFVEGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMITYKAERIGIEVKKVNPAYTSQTCSRCGNVDKENRQTQDKFKCTKCEFELNADHNAAINISRSKKYVK

SEQ ID NO: 427 - protein, 403 aa, unidentified
  source 1..403: note = uncultivated organism; MG119-131 Effector
MITVRKIKLTVIGTDEERKQGYKFIRDSQYAQYQGLNLAMGILSSAYLESNRDIKSDIFKNAQKSLTNSNPLFEGITFGTGIDTKSSITQRVKKDFKTSLKNGLAKGERTITNYKRNYPLMTRGRDLKFMYEGDDILIKWVNKIIFKVVLGATKRKENSIELQHTLHKVIDKEYKVMQSSLCFDKNNNLILNLTLDIPYTPKKNFVEGRVLGVDLGIKYPAYVCLNDDTYKREHLGSIDDFMKVREQMQQRARNLQQALKLTKGGKGRTKKTQALNRLKDKERNWAKTYNHQLSKKIVDFAIKHQCEFINMEKLTKDGFGDKLLRTWSYYELQNMIEYKAERVGITVRYIDPAYTSQTCCICGHIDKDNRQTQEKFKCTECGFELNADHNASINIARSEKFIK

SEQ ID NO: 428 - protein, 403 aa, unidentified
  source 1..403: note = uncultivated organism; MG119-132 Effector
MITVRKLKLTVIGTDEERKQGYKFIRDSQYAQYQGLNLAMGILSSAYLESNRDIKSDTFKNAQKSLTNSNPLFEGIAFGTGIDTKSSITQRVKKDFKTSLKNGLAKGERTITNYKRNYPLMTRGRDLKFMYEGDDILIKWVNKIIFKVVLGATKRKENSIELQHTLHKVIDKEYKVMQSSLCFDKNNNLILNLTLDIPYTPKKNFVEGRVLGVDLGIKYPAYVCLNDDTYKREHLGSIDDFMKVREQMQQRARNLQQALKLTKGGKGRTKKTQALNRLKDKERNWAKTYNHQLSKKIVDFAIKHQCEFINMEKLTKDGFGDKLLRTWSYYELQNMIEYKAERVGITVRYIDPAYTSQTCCICGHIDKDNRQTQEKFKCTECGFELNADHNASINIARSEKFIK

SEQ ID NO: 429 - protein, 403 aa, unidentified
  source 1..403: note = uncultivated organism; MG119-133 Effector
MAITVRKLKLTIVNEDENVRREQYEFIRNSQYAQYQALNLGMGYIMTGYLACNRDIKSEDFKEWQKGLKNSNPIFKGIEFGKGVDTLSAVTQKVKKDFSTSLKNGMAKGERASTNYKRSFPLMTRGRSLTFYEENGEIYIKWVNKIVFKVVFNARKENTLELQHTLNKVITGEYKLSQSSLEFDRNNNLILNLTMDIPFKQQEEFVEGRVLGVDLGVAVPAYICLNDDTYKRQGLGSYEDFFKVRQQMKARRERLQKQLKLVKGGKGRNKKLKALNQFKEKERNYAKTYNHNLSRAIVDFAKQHKCGQINIEHLTGESFDNKLLGSWSYYELQQMITYKAERIGIEVKKVNPAYTSQTCSRCGNVDKENRQTQEKFKCTKCNFELNADHNAAINISRSKKYVK

SEQ ID NO: 430 - protein, 467 aa, unidentified
  source 1..467: note = uncultivated organism; MG119-134 Effector
MITVRKLKLTIVGDEETRNQQYKIIRDEQYQQYRALNLCMSLLNTHNILNTCNSGAENKLNSQIEKLDKKIEKNKNDLKKDNLKDNKIKKINESIQTLTKEKEKLQQEYLKSSKYRSNIDKKVKEMYIDDLYTVVQSQVNFKSKDMMSLVTQRAKKDFATALKNGMAKGERSLTNYKRDFPLMTRGERWLKFEYDEDSDDIYINWLHGIRFKVVLGYKKNENSIELRHTLHKVINKEYKICDSSMQFDRNNNLILNLTLDIPLNIKNEHIEGRILGVDLGIKYPAYVCLSDDTYKRKSIGCAEDFIKFREQIRARRYRLQKQLSMVKGGKGRNKKLQALDRIKDKERNFVKTYNHMISKNVVEFAKNHKCESINLEKLTKDGFPNMILSKWSYYELQNMIEYKAEREGISVKYVDPAYTSQTCSKCGHVDKENRTSQEKFKCIECGFELNADHNAAINIARSNNYVK

SEQ ID NO: 431 - protein, 403 aa, unidentified
  source 1..403: note = uncultivated organism; MG119-135 Effector
MSITVRKLKLIIVNDNEEERKAQYKFIRDAQYSQYQALNLGMGYLMTGYLAYNRDIKSEEFKAWRKELKNSNPIFKDVKCGKGIDTLSAVTQKVKKDFSTALKNGMAKGERTSTNYKRTFPLMTRGRDLKFYAEGSDIYIKWVNKITFKVVFNARKENTIELQHTLHKIINEEYKLSQSSIEFDRNNNLMLNLTIDMPFNECDNLIKDRVLGVDLGIKVPALVCLNDDTYKRQSIGSYEDFFKVRQQFKSRREKLQRQLKMTKGGKGRKQKLKAFNKFKEKERNYVKTYNHNISKCIIEFAKKHKCSQINIENLTGENFDSKVLGSWSYYELQQMITYKAERVGIIVKKVDPAYTSQTCSVCGHIDKENRQTQEKFKCTMCGFELNADHNAAINIARSNKYVK

SEQ ID NO: 432 - protein, 145 aa, unidentified
  source 1..145: note = Uncultivated organism; MG119-1_sgRNA1
AUAGGGCGACUUGGCGUCCUUAAACCGAGAAGUGCAUCAGAAGGUUUACAUAGAUGUAACACUCGGUAAGGUAAAUCCGUAAGGAUAAUCCACCCCGGCGAAAUGCCGGCCACUAAAAUGCAUAGAAAUAUGCACAAGUGGAAGU

SEQ ID NO: 433 - skipped

SEQ ID NO: 434 - RNA, 129 nt, unidentified
  source 1..129: mol_type = other RNA; note = Uncultivated organism; MG119-2_sgRNA1_Mutant1
tacacatcattagatgtgagggcaatttaatgtcctaatattgagggtattaggtaacaacactttgttctaatagaacagtgtctaataaccactcgataagaccatatgaaagtatggttataaaac

SEQ ID NO: 435 - skipped

SEQ ID NO: 436 - RNA, 156 nt, unidentified
  source 1..156: mol_type = other RNA; note = Uncultivated organism; MG119-3_sgRNA1_Mutant1
gttaaaattctagatatgattattatattccctttatgggaagggtgtgttggaacgccttagtttgaggtttgaatataaaaagtgaaacaaatggagatttgtaacaccttcaaacactcaaaaaattcatttagaaataaatgaatatccaac

SEQ ID NO: 437 - skipped

SEQ ID NO: 438 - RNA, 117 nt, unidentified
  source 1..117: mol_type = other RNA; note = Uncultivated organism, MG119-4_sgRNA1
ttagaaaagaaattttctaattggggctgctggaagtccttaacctgagataggaaactatactcgggaagtacgtcaaaacattgagaaatcattgttttgaatggtatatccaac

SEQ ID NO: 439 - skipped

SEQ ID NO: 440 - RNA, 143 nt, unidentified
  source 1..143: mol_type = other RNA; note = Uncultivated organism; MG119-10_sgRNA1
tattgcgatgagtaaaaaaatcgtagaaagataattagagaagaaattttctaattggggctgctggaagtccttaacctgagataggaaactatactcgggaagtacgtcaaaacagaaatgttttgaatggtatatccaac

SEQ ID NO: 441 - skipped

SEQ ID NO: 442 - RNA, 132 nt, unidentified
  source 1..132: mol_type = other RNA; note = Uncultivated organism; MG119-19_sgRNA1
caagtagatgcgaaagataaagtatgtaagttttgaataacaatattattcaaattggggcagttggatgcccttatgctgagggattattccactcggcaagtaccaatagaaatatgaggtacatccaac

SEQ ID NO: 443 - skipped

SEQ ID NO: 444 - RNA, 157 nt, unidentified
  source 1..157: mol_type = other RNA; note = Uncultivated organism; MG119-27_sgRNA2_Mutant2
ttgatataacgtatatgaatcttctattcccctgtgggggaagggtgtgttggaacgccttggtttgaggtttgaaatagaaaaaagaaatacgggaatgtattatcgatttcaggcactcaaaaaattcacttaagaaattaagtgaatatccaac

SEQ ID NO: 445 - skipped

SEQ ID NO: 446 - RNA, 152 nt, unidentified
  source 1..152: mol_type = other RNA; note = Uncultivated organism; MG119-28_sgRNA2
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaac

SEQ ID NO: 447 - skipped

SEQ ID NO: 448 - RNA, 140 nt, unidentified
  source 1..140: mol_type = other RNA; note = Uncultivated organism; MG119-32_sgRNA1
atatagtcccattagggattgggcgtgttggaacgccttagtttgaggtttgaaaccaaaagcggtgtagatggaggtctgtaccaccttcaagcactcaaaaaatccacttatatgaaaatataaggggatatccaacg

SEQ ID NO: 449 - skipped

SEQ ID NO: 450 - RNA, 147 nt, unidentified
  source 1..147: mol_type = other RNA; note = Uncultivated organism; MG119-54_sgRNA1
aacagatatacgacacgaagtatatttacatggggctgttggaggcgccttagtttgaggttcagacataaaaaatcgttatgacagtcggttataacgatgtctgcgcactcgaaaaattcatctcgaaagggatgaatatccaac

SEQ ID NO: 451 - skipped

SEQ ID NO: 452 - RNA, 112 nt, unidentified
  source 1..112: mol_type = other RNA; note = Uncultivated organism; MG119-64_sgRNA2
ggggcagttggatgcccttagcctgaggaatttatttcacttgggaagtaccattcttattgaatgacagctagttatgttactgaaaagtaaaatgtttggtatatccaac

SEQ ID NO: 453 - skipped

SEQ ID NO: 454 - RNA, 133 nt, unidentified
  source 1..133: mol_type = other RNA; note = Uncultivated organism; MG119-72_sgRNA1
aaatatttcattaagtaccgaatttaaaaaataggattgcagaaagtgcaattaggctggttgtgcagccttaatctgagggattaatccactcggaaagtacctttattgaaaaatgaaaggtattcacaac

SEQ ID NO: 455 - skipped

SEQ ID NO: 456 - RNA, 104 nt, unidentified
  source 1..104: mol_type = other RNA; note = Uncultivated organism; MG119-83_sgRNA1
aaggaataggtcataaggcctatggggctgttggaagcccttagcctgaggactttagtcactcgggaattaccttaatttgaaaaaatgaaggcaaatccaac

SEQ ID NO: 457 - skipped

SEQ ID NO: 458 - RNA, 156 nt, unidentified
  source 1..156: mol_type = other RNA; note = Uncultivated organism; MG119-97_sgRNA1_Mutant1
tcatggggtgatttggcatcctacaatcgagatagtgtatataagactatattaatatagcaataaaaacactcgataaggctctaagaattctccaaccgaattcttaaccaccttgtccgaaaaggacaacaatatatgaaaatatattgaaag

SEQ ID NO: 459 - skipped

SEQ ID NO: 460 - RNA, 155 nt, unidentified
  source 1..155: mol_type = other RNA; note = Uncultivated organism; MG119-109_sgRNA1
attcagggcggctcgacgtcctaaaatcgagaaagtatgtataagtctttataatatggagcagcagatactctcggtaaggtataaattccacaatgtaaatcctgtggagaccctggttattgtgtagaaatgcgagagtgtgccgggtacga

SEQ ID NO: 461 - skipped

SEQ ID NO: 462 - RNA, 170 nt, unidentified
  source 1..170: mol_type = other RNA; note = Uncultivated organism; MG119-118_sgRNA1_Mutant2
cggcagggcgacttagcgtccaaaagtcgagtgatttgacgataatatgtcaaactttaatagtaatagtttactctcgataaggtaacaaattataagatttgttgaaactgcatatataaaatccatatgcacatctcgtatttgaaaagatatgaaatgtaagttga

SEQ ID NO: 463 - skipped

SEQ ID NO: 464 - RNA, 141 nt, unidentified
  source 1..141: mol_type = other RNA; note = Uncultivated organism; MG119-121_sgRNA1_Mutant1
attgagggcgactgtgcgtcctaagtgtgagggaatttaccaagtgaatatccactcataaagtcttgaatatattgatattacaagacatcaccctgcaagtagcaggtataatactctagaaatagagtattataagta

SEQ ID NO: 465 - skipped

SEQ ID NO: 466 - RNA, 144 nt, unidentified
  source 1..144: mol_type = other RNA; note = Uncultivated organism; MG119-125_sgRNA1
taaacaattgaataaaaaatattatatacgggcgattcaccgtccgaaggttgaggatacgtaagaatataagtctaggctttggccaacagatttccgtatcactcgataaatcacatagtgaaaactatgtggtatgtgaat

SEQ ID NO: 467 - skipped

SEQ ID NO: 468 - RNA, 135 nt, unidentified
  source 1..135: mol_type = other RNA; note = Uncultivated organism; MG119-128_sgRNA2_Mutant1
ttagttacacatcattagatgtgagggcaatattatgtcctaatattgagggtattaggtaacaacactttgttataatagaacagtgtctaataaccactcgataagaccatatgaaagtatggttataaaact

SEQ ID NO: 469 - skipped

SEQ ID NO: 470 - RNA, 133 nt, unidentified
  source 1..133: mol_type = other RNA; note = Uncultivated organism; MG119-129_sgRNA1_Mutant1
tacacatcattagatgtgagggcaattatatgtcctaatggtgaggatgcagattaaagcattaggttcatgaaccaacaatgtactgtaatcactcgctaaatcgtgtaagaaattacatggtactaaaaca

SEQ ID NO: 471 - skipped

SEQ ID NO: 472 - RNA, 144 nt, unidentified
  source 1..144: mol_type = other RNA; note = Uncultivated organism; MG119-133_sgRNA1_Mutant1
taaataaattaaaaatacatattatatacgggtgatttaccatccgaaggtcgagggtgtgggtttaatatctgggtttagaccaaaaggattctcacatcactcgataaatcatatggttgaaaaactatgtgatatgtaaat

SEQ ID NO: 473 - skipped

SEQ ID NO: 474 - RNA, 127 nt, unidentified
  source 1..127: mol_type = other RNA; note = Uncultivated organism; MG119-136_sgRNA1_Mutant2
attataagttatattataattgggctagttgcatagcctttcgtgaggttaacgaattaaatatattaattcgttgaaaaacactcataaactactgtaaattgaaaaattgtatggtagatgcaac

SEQ ID NO: 475 - skipped

SEQ ID NO: 476 - protein, 464 aa, unidentified
  source 1..464: note = Uncultivated organism; MG119-136 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGRDKSNNASIIEKTISGEYKFCDSNVVVEKNKMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 477 - protein, 446 aa, unidentified
  source 1..446: note = Uncultivated organism; MG119-139 effector
MGVSCKKLQIIPVGDDNEIKRVYEYIRNGIYSQYRILNTYMSQVGTLYYKYDRDYKSPGFKDEYNAIFRNTNTAIHDIEQAKGLGMAGNCGMRVKSDFSTALKNGLARGERQLPFYKRDFPLLVPGRFLVFSEKEEDYYDDNGELKTVVNYYIKFVNGIVFKVVFGSKGRRDYRLTSLLSGIVNDVDHYKVCGSSIQISGKKIILNLTTNVEKETVPFIPIKDKKMGIVLGYDQPLVAALSNDDKEYYIGTDIVESIVERRTAIQAYRHRLQAMQTTAKSGHGRKRRMRRVDQQGHYERNVMRNYNHILSSRVVSFAVKNKVETIFIDDISKDTLVDEPILLRNWSYYQLVEFITYKAKEVGITVITVDFKKNKKEDGEDDTDASNKHDFFDRCCVCGEQIDGLAELYDDISVYKDESSFTCPHCHKKVKVRYNKAKNLTYYKNKK

SEQ ID NO: 478 - protein, 525 aa, unidentified
  source 1..525: note = Uncultivated organism; MG119-140 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVAIGLVQPAVCALNNDQYVRLPIGDERDLLDNKLQIQERRRRLQSALRETSGGHGRKKKMQALNRIAKTESNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 479 - protein, 525 aa, unidentified
  source 1..525: note = Uncultivated organism; MG119-141 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 480 - protein, 535 aa, unidentified
  source 1..535: note = Uncultivated organism; MG119-142 effector
MAAKDNTMVITRKIALIPVVSERKEWKKRINAFLEKDFPRKIEAKKKQIKNTSKPENKDGYKLQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPSFKIDSPFTIAKAYFDLYHEYEDIAELKKRIYDSDCKLYCGLGNKGIPTIANFKVNIGHKGNRDELISTLLRIFTGEYEICGSTMQISKTKIILNLSIRIPKTQLELDEDTVVGVDLGVAIPAVCSLNNNKYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQVCSFCGHWEDGQRKDQATFVCGNPNCESHKLHMVNADYNASRNIAMSTLFTSDKFQFCKKTMQNAAEYYGIQLDNEDQLAA

SEQ ID NO: 481 - protein, 552 aa, unidentified
  source 1..552: note = Uncultivated organism; MG119-143 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTLNYAYRKKGSNKGSLFDDTEINSILNAYGIAWNQELTKEIKDLVKNGALEGKVSLTNYKLDSPFTIAKAHFSFDHDYDSFEELCEHIDDSDCKMYMNYGGDNKKDGTNPASLARFRINLGQGKNRNELKSTLLKVYSGEYQYCGSSIQISKNKIILNLSMKIPKIETELDENTVCGVDLGIAVPAMCALNNDMYKRLAIGSADDFLRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAARYGIVVRKINPCYTSQICSVCGNWEPDQRKTQAKFECANEECASYDKYKYGFNADFNAARNIAMSTLFMETGEVTEKKKEEARKYYGIEEKYQKYQASLKEKDDKVA

SEQ ID NO: 482 - protein, 579 aa, unidentified
  source 1..579: note = Uncultivated organism; MG119-144 effector
MKKNNKESNTYIKAMKIKIIPICDIKEENLRDKYYKELYQYLRDSIWAQNAALNYGLAMTRDAYVLHKNEDKIKDIYFKLAHQTPNPSMASEARLKEVYEAAPITQEYIDNKVEEFKKSNSKKKKPLTDEGVKKKTDSINNMYKKFIGLSKEDIQELIDKLDDYCAYPEEIYEKFANGFSTPAYVTQQIKDYWNTEGVMTKVIYSMSDKLRSVKNSNPLTIPPNLFYNKKNDLIGITHSYNTYLEFVEALMNAYDANLFLELPYKKGYDKMKFKLILGNPCKSHEVRISLQKIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGKGYTLNADYNAARNIAMCDLFMDNKSEITEEDKKIAREKYGIPEPVKKEEKIAA

SEQ ID NO: 483 - protein, 535 aa, unidentified
  source 1..535: note = Uncultivated organism; MG119-145 effector
MTAKDNTMVITRKIALIPVASERKEWKKRINSFLEKDFPRKIEAKKKQIKNTSKPEKKDGYKQQLTELEKQYEDFKENGIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPNFKIDSPFTIAKAYFDLYHEYEDITELKKNIQNSDCKLYCGLGNKGIPTIANFKVNIGHKGNRDELISTLLRIFTGEYEICGSTMQISKTKIILNLSIRIPKTKLELDEDTVVGVDLGVAIPAVCSLNNNKYKREYIGSSDDFIRMRTKLQHEKQQLQKALKMSKGGHGRKRKLLALERCKKKEYNFVETYCHKVSKKVVDFAVKNKAKYINVENLSGYDSSKFILRNWSFYKLQQYIEYKAAKYGIEVRKVNPSFTSQVCSFCGHWEDGQRKDQATFVCGNPNCESHKLHMVNADYNASRNIAMSTLFTSDKFQFCKKTMQDAAEYYGIQLNNEDQLAA

SEQ ID NO: 484 - protein, 522 aa, unidentified
  source 1..522: note = Uncultivated organism; MG119-146 effector
MENNITITRKYALIPEFSDRKEWKKRVYDFMINDLEQKIDYRNKKKQDTSELESQLEYIKNGGDFTRSMVNNYTYSLVRKAMEEETRRKNYILSWIFSEMRANRIDQMESLKDKFKFVSDTINYAYRKAGSNKGSLFDETEIHCILKSYGIAFSQELTKEIKELVKNGVLEGKVVIPTYKLDSPFTIAKSHFSFEHDYDSFEELCEHINDSDCKMYMNYGGDNRKDGINPASIARFRISLGHGKNKDELKSTLLKVYSGEYQYCGSSIQITKNKIILNLTMKIPKIETKLDENTVVGVDLGIAVPAMCALNNNMYERLAIGSADDFLRTRTKLQSQRRRLQKSLKNSNGGHGRNKKLKVLERLGKSETHFAETYCHMVSKRIVDFALKNNAKYINIENLNGYNTSSFILRNWSFYKLQQYITYKAERYGIVVRKINPCYTSQVCSVCGNWEDGQRKTQASFECANPKCESHKKYKYGFNADFNAARNIAMSTLFMEDGEVTEKKKEEAREYYGIKKENSEAV

SEQ ID NO: 485 - protein, 521 aa, unidentified
  source 1..521: note = Uncultivated organism; MG119-147 effector
MAKSSENVLTRKVQLIPEISDRKEYTKRLTKYINDSYSEKIEKKRNKKFNRKDYDSLEKFKEAKEKNESEIQNLQDALADLATGTITKKMVDDYTYGLVREAMESESSRKNYILSYVYSQLITSDYANMDKEERKQFFKDIWNPCCRKKGSNLGSIFDNTDIDNILGGYGTGFSQDLKSKINNAIKDGLLEGKVSLPMYKKDSPFSIASKFFNFYHTYESFDELEKNIEKPNCQLFMAFGGNGNPTIANFRVNLGCGSGAKKRKDNSICQTMLKVLAGEYKICGSSIQISKGKIMLNLSMDIPIKEHKLDPDICLGVDVGYVIPAMCGLNKGKLAFRDARFGDGTDLNGRRKRIREQKKRMQVGIATNKGGHGRAKKIKALDRFREKERNFARTYNHKVSHDIVNLALKNNAMYINLENLKGITSDEKKKRELGDWSYFQLQDFITYKAKQHGIIVRKVNPYRTSQTCSCCGHWEEGQRVDRDTFICKNSECEQFGKKIHADLNAAINIAKSTDFSVKKDA

SEQ ID NO: 486 - protein, 576 aa, unidentified
  source 1..576: note = Uncultivated organism; MG119-148 effector
MNTINNTYIRTLKFNLNLTPKFETEEDNKKYINDVYSYLRDAMWAQNRAMNIVLDRTKEAYTLGRGMNRVKEIYYSYSHQKPISNDKKESFLESLLQYAPIDDQFVKNEVKKLRKFYESKKKPPKEETVSKNCEALKNKYIKYVGKSKDDIKRELDLLENYCAYPEDIYEKFANGLSTPAYIKQKVESYWKQDGIKTKVIYSMDENLRRIKDAPLFIPPNIFYNKKDELIGLVYDYTDYISFLEDLENKRNVNIYLSIPYKKGEDKLKFKLVLGNPHKSRDSRLSIKRIFEEEYRIKGSSIGFTKNKETGKNTNLTLYLTVEVPQNKDNTLDENVVVGVDVGIAIPCVCALNNDKYTRENIGSYDTLFAKRTQFKMQRSRLNSQLKLSKGGHGRKRKLKKLELLSGKEKNYADTECRKYASDVIKFCLKNHAKYINLEHLKGYRENPKVLAGWSFYKIQTYIEQAAEKHGIIVRKINPCYTSQICSVCGNWHPENRPKGKLGQAYFNCHNIDCKTHNTDLYKYGINADFNAARNIAMSTLFITDSDEITKKHWKEAREYYGIDESDDKEEKLNKVA

SEQ ID NO: 487 - protein, 675 aa, unidentified
  source 1..675: note = Uncultivated organism; MG119-149 effector
MSKKNTIVEITFTNGVKKEVVLPPKASTIPSKKIRMIEQEKNYGIRKVVIPEGITKITSRSFKDILSLEEVVLPDSLTVVGNSAFQNCISLKIIKIPNSVTAIGNSAFQNCAELTDIDIPDSVTVIGHSAFQNCIGLISVTIGSNVTEIGDYAFSGCSGLTGIDIPHSVNEIGSYAFNDCTGLVSITIPNSVLRIGNGAFEGCTGLTGTENSSNRTNGNVVIISRKIQIVPIGDTKNCAPIHEYISEAMDAQYRAKNLLMGHIGSMYYKNEQKMTKDLKSGVLYSGNPVLSSISFPIGLDLNSSIRNQIRQEIDSALKNGVAKGERSLSSYKRNNPVPTNGRNVHFNFEFDEKDIENYKDDPTNPLNEKNLLMHMRVTINWVNNKGFIVNPGSSMDIRRILFRLYTGEYKHGQAKLQILKDTNDKKNLEKIMMILSLVVPNTSIELDENTVVGVDIGAKNPAVCALNTDENKRLYCGDNFAMLKAHTEFQGRKKEIAHTSLFNCSQHGYRKRNKRLNQAKKHEQNKTKNISHEIAKQVVEFAIENNAKYINVENLEGLFETPGNSADRKVWSYYQIQTLIMQKAARYGIEMRKVESRNTSRICSFCGSDAEDQLRDRDTFICKNPDCVSHKLHHGFVESDFNAARNIAKSTEWSKGKAKKSSKEKDKNKDESKKQ

SEQ ID NO: 488 - protein, 512 aa, unidentified
  source 1..512: note = Uncultivated organism; MG119-150 effector
MDTMIITRKFVLVPVASCKKEWVKKATAYIVSDTEDKLDFFKEKLKKVEKEMKKAKDSEQLSKEYNRLCSKKEEYESLLKDAEDGVFNQKAINNYTYHLVREAMESEARRKNYILSWAFSEMIANGVPYMETYKEKCKFINEMIKPAYRTKGSKKGSLFDDSEIQNILGGYGVSFKQELTQKLKDCVKDGLLEGKVVLPNYKLDSPFTVEKTHVSLSHDFETYEELCEHIGKKDGKIYLNYGGHGESTIAKFAIDTGSNKNKEELNTTLLRLISGAYEVCGSSIGLCKDGKKIVMNLSMKIPKTELELDEKTVVGVDLGIAVPAYCALNNNIYSKKAIGSAAEFLRIRTQLQSQYRRLQKQVAANNGGHGRNKKMKPLDRFREKERNFVKTYNHYVSKEVVEFAVKNKAKYINMEDLSGFKGNDDRILRNWSYYELQQFIEYKAAKYGIEVRYINPYYTSQVCSCCGHWEEGQRMDQAHFVCKKCGTELNADYNAARNISMSTEFKKQKLAA

SEQ ID NO: 489 - protein, 525 aa, unidentified
  source 1..525: note = Uncultivated organism; MG119-151 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 490 - protein, 587 aa, unidentified
  source 1..587: note = Uncultivated organism; MG119-152 effector
MLDKKTNATSSKIITITESQKLELYPLPCIEVKQWNGRVNENFDYQKDLLEKSIKRKKGWINNYEKRLNEAERKLKDYISNNHIDNEDKDKNIKALNKQINECNKKIRKYEKECKEHMEKINAYQKEQDRFNEIINHDNLTKDDTEFINKTAKNYTYSLIRRACKSEAARKNATLSYTYSECIRNNVADMEDFGDICSAIDEILKHCNRTRNGLSDDLNIDNPLGGYGISYNQNLRSSFINMVKKGLFDGRVSLVNYKSDSPVSIAGAHYNFFPSFDLIGFEDFYDLKRVMQKENGVIYLNVGSNGLPSIARFRVVIGSKNHKSDSIYAFLYNVLKNNYPTGGSSIQIENNKIMLNLSYKKSSQEIDTLKDDVVVGVDVGISIPAVCALNNSKGYRWDIGDKKELLRIRTKLQEQKKRITNKKDLCSSGKEKKKINKASFRLTKHIKHWKRTYNHMLSRRIIKFAIKNNARYINLENISKFDKKEKERRFLKEWSYYELQKYIEYKAKEYGIIVRYVKAENTSTTCSCCGNTDEEQRNKSIFVCKNHECKQYGKKVNSDFNAARNIALSTDFFSKKELNEESLENED

SEQ ID NO: 491 - protein, 543 aa, unidentified
  source 1..543: note = Uncultivated organism; MG119-153 effector
MDSNNTQIVTRKLVIRPTFSERKEWKKKVTEFTLKDLEEQISKYKEWIENLKSNKKIDKEEKERRKQKYTDLLTNAENSLNQFKDREEITQKMVNRYTETLVKESMESEARRKNYILTWAYFSMRANGVQHMQPKEQKKFIREMLKPAYRVKNSSKGSIFDDVEIDNILGGYGMAFNQELTNKIIDSVEKGLFEGGKDSLPSYKYDSPFTVAKSTMGFSHDYDSFEELCEHIKEKDCNLYFDFGGNSDATIARFIIDVGTNRNKDELLITLCRVYSGEYEYCGSNIQFDKTGKKIILNLTIKIPQKNCDLNENTVVGVDLGIKIPAVCALNTNPYAREFIGSVDDYLRVRTQIQNQRRRLSIQLRNTKGGHGRKKKLKALDRYTSRERNFVKNYNHMVSSKIIKFALKYNAKYINLECLKGYDADKKALRNWSYYELQSMIEYKANKYGIVVRKINPCYTSQVCSECGHYEEGQRVSQEKFVCGNDDCRFHNTKSGYINADFNAARNIAKSELFLDSEVTKKRIEEAARYYNIPYESKNTDEV

SEQ ID NO: 492 - protein, 525 aa, unidentified
  source 1..525: note = Uncultivated organism; MG119-154 effector
MSEKMTMITKKCVLIPERSDSSLWERRVYDFTVKDYEQRISFYESQLKSKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKTMDRAAEYYGISEKYKA

SEQ ID NO: 493 - protein, 534 aa, unidentified
  source 1..534: note = Uncultivated organism; MG119-155 effector
MSEKMTMITKKCVLIPERSDSSLWERRVYDFTVKDYEQRISFYESQLESKKKEVRENAKKRLEALNKELDAFLENPEITQNVINNYTYGLVHTAMSEESARKNYIIEYVKSILIAEHAYAMDFKDRNKRITQILQYAYRKKGSNKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFRIDLGHGRNRDKLMATLLKVYSGEYAYCGSSIQISKNKIVLNLSLQIPKEEKDELDEDTVVGVALGLVQPAVCALNNDQYVRLPIGDERDLLDNKLQIQERRRRLQSALRETSGGHGRKKKMQALNRMAKTEKNFTETYCHMVSKRIVEFALQNHAKYINMENLSGYNTDKFILWNSYASKIIMYTTYKAGHYGIEVRLVNPCFNAQVCSICGNWTPEQKISRTEFVCADPDCKSHTKYKKGFTADFNHARNVAMSTLFMENGSFSKKSMDEAADYYGISEKYKAHIGNEPKVE

SEQ ID NO: 494 - protein, 551 aa, unidentified
  source 1..551: note = Uncultivated organism; MG119-156 effector
MPKKKSNTKTKKDNKTKTPKVTSVVFCKKLKLKPVPVLDNKKWAKRVLAYFAYHINYQSELIEKYELWMQDATKTKKEAYKKQQAIAEKKIEKLKADAKRFENLFTNGLETQNDEDFIIKIAKDYTYDFIRKACKSEAQRKNAALSFTFSECLNRNIKDIEDIKERSRAISDILNFCNRVKSKSVSEQPNSLADMIDIENPLGGYGYDYKQAIRSNMLDMIKDGLLEGKIALKCYRMDGPVTIGADHFSITPDFDLIGTKDYTDLQRIIKKENGRLYVNIGGLGNPTIAQFEIILGSKQKARDDLFSLFYDILKKNCTFGGSSVQITDRHEIILNLTIKKDKELKNKLKDDVIVGVDVGINTPAMCALNKKKDFYLVLGSKDDFLRMRGQLKAQRKRLDCARRLCTTAEEKKRLLAKSERLTKHTDKWAQTYNHMISKRIITFALRNNAGIIRLENLSKFSDEQKQNNLLEDWSYFQLQNYIEYKAKAEGIKVEYVNPENTSTTCACCGETENTERRKNSIFICHNPKCKNCDKEVNADLNAAKNIASTEP

SEQ ID NO: 495 - protein, 533 aa, unidentified
  source 1..533: note = Uncultivated organism; MG119-157 effector
MDDDKVIITRKYTLIPEFSDRKEWDKRVRSFIKTDSENKIEYLIKKRTTVKGKAEKTKIDTRISTYKERIEIIDSGGEITQKMVNDYTYNLIRTAMEEEARRKNYILSWIFSEMRLHRVDCMETLKEKFKFISDTINYAYRKAGSSKGSLFDDTDITSILKSYGIAFSQQLTAEIKDLVKAGLLEGKVTLTNYKLDSPFTIARSHFSFDHDYDSFEELCEHVNDSDCKMYVNYGGDNTKTGKNPASIARFRINLGHGKNRDELKSILLKVYSGEYDYRGSSIQISGNKIILNLSMAIPKQTMELDEETIVGVDMGIAVPAMCALNNNLYEREAIGNADDFLRMRTKMQKQRRRLQKSLRNTSGGHGRKKKLRSLDRVGKSEAHFVETYCHMVSKRVVDFAVRHHAKYINVENLTGYDTSEFILRNWSYYKLQQYITYKAAKYGIVVRKVNPCYTSQMCSICGSYEPGQRKSQSVFECANENCNSHTKYKHGFNADFNAARNIAMTTLWMETGQVTEKSKQEAREYYGIAEEVA

SEQ ID NO: 496 - protein, 541 aa, unidentified
  source 1..541: note = Uncultivated organism; MG119-158 effector
MAKKQDDNTMTIARKITLIPVASDNNKWKKRVDNFLEKDFPRKIEAKKKQIKNTSKPERKDGYKQQLVELEKQYEEFKENGIKEYTHKMASDYTYDIVRKAMDSEARRKNYILSYIYTKMIQEEVANLPTLTEKNKWVSANVKECYRKAGNKNGSIFTNVDIDNPLAGYGSDFKQALTGKIKNLIKDGVLEGKVSVPNYKFDSPFTLTNQNFGIFTDCKDITELKKNIGKSTCPVYVCLGKNGSPTIAKFKINFGYKQNKNKAELISTIIKIFTGEYSVGGSTFGIDDDKIEMNLSITMQKQKMDLDENTVVGVDLGLAVPAVCALNNNEYDKQYIGSGNDLVSRRTKFQNEYTQLQKALRLAKGGHGRKRKLLALERLKEKENNFVDTYCHRVSKKVVDYAIKNRAKYINIENLKGYDSSEFALRNWSFYKLQQYITYKAEQCGIIVRKINPSFTSQVCSFCGHWEKGQRKDQATFKCKNPNCKSHKLYTVNADYNAARNIAMSTQFTDDKFKCSKETIQAAADYYGIVLEDDNNNDKAA

SEQ ID NO: 497 - protein, 525 aa, unidentified
  source 1..525: note = Uncultivated organism; MG119-159 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKTMDRAAEYYGISEKYKA

SEQ ID NO: 498 - protein, 552 aa, unidentified
  source 1..552: note = Uncultivated organism; MG119-160 effector
MKKNDDNKVIIARKYNIIPTCSEKKEWKKKVYDFTIEDLNKKIIDFEKKIKKQKDKKKKENYIQKLNTYKESLASLSVENPEFTQTMINNYTYNLIRSAMEEEARKKNYILTWIFSEMIANGVQYMPTLQEKNKFVSDTINYARRVKGSSKGSLFDNTEITNTLGAYGLVFTKELKNKIQRDIKDGLLDGKVSLSNYKLDSPCTIAKDHMKFTSDYDTFEELCEHIDDNDLKMYFNYGSKGEPTIAKFKINLGHGKNRAELKSTLLKVYSGEYEYCGSSIQIAKTKIILNLTMKIPKKELDVLNEDTVVGVDLGIKIPAMCALNNDAYSRQAIGSSDDFLVKREQFQKQRERLQKALKNTKGGHGRKKKLQALNRVKNAEAHFVETYNHMVSKRVVDFALKYHAKYINLENLTGYDTSDKILRNWSYYKLQTYITYKAKKYGIIVRKINPCYTSQICSVCGNWHPENRPKGDKGQGYFNCHNEECKSHDKEKYKYGLNADFNAARNIAMSTLWMDEGKVTEKKKEEAREYYGIEEEYQKYLEEKQKNKKKAA

SEQ ID NO: 499 - protein, 520 aa, unidentified
  source 1..520: note = Uncultivated organism; MG119-161 effector
MIPVASERKEWKKRINAFLEKDFPRKIEAKKKQIKNTTKPENKDGYKQQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIENPLGGYGTCFSQILASKIKDLVKQGLLDGKVSIPNFKIDSPFTIAKAYFDLYHEYEDITELKKNIQNSDCKLYCGLGNKGIPTIANFKVNIGHKGNRDELISTLLRIFAGEYEICGSTMQISKTKIILNLSIRIPKTQLELDEDTVVGVDLGVAIPAVCSLNNNNYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQLCSFCGHWEDGQRKDQATFVCGNPNCESHKLHMVNADYNASRNIAMSTLFTSDKFQFCKKTMQDAAEYYGIQLDNEDQLAA

SEQ ID NO: 500 - protein, 525 aa, unidentified
  source 1..525: note = Uncultivated organism; MG119-162 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVAIGLVQPAVCALNNDQYVRLPIGDERDLLDNKLQIQERRRRLQSALRETSGGHGRKKKMQALNRIAKTESNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTKYKKGFTADFNHARNVAMSTLFMENSSFSKKSMDEAAEYYGISEKYKA

SEQ ID NO: 501 - protein, 525 aa, unidentified
  source 1..525: note = Uncultivated organism; MG119-163 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 502 - protein, 539 aa, unidentified
  source 1..539: note = Uncultivated organism; MG119-164 effector
MNSNNTQIVTRKIVIRPTFSERKEWKKKVTEFTLKDLEEQISKYEEWIENLKSNKKIDKEEKERRKQKYTDLLTNAENSLNQFKDNEEITQKMVNRYTETLVRESMESEARRKNYILTWAYFSMIANGVPDMQLKEQKKFIREMLKPAYRVKKSSKGSIFDDVNIDNILGGYGISFNQELTNKIIDSVEKGLFEGGKDSLPSYKYDSPFTVAKSAMGFSHDYDSFEELCEHIKKKDCNLYFDFGSNSDATIARFIIDVGVNRNKDELLTTLCRVYSGEYEYCGSSIQFDKTGKKIILNLTVKMPQKNCDLNENTVVGVDLGIKIPAVCALNTNPYAREFIGSVDDYLRVRTQIQNQRRRLSIQLRNTKGGHGRKKKLKALDRYTSRERNFVKNYNHMVSSRIIKFALKYNAKYINLECLKGYDTDKKVLRNWSYYELQSMIEYKANKYGIVVRKINPCYTSQVCSECGHYEEGQRVSQEKFICGNDGCRFHNAKNGYINADFNAARNIAKSELFLDSEVTKKRIEEAAKYYNIHINDEV

SEQ ID NO: 503 - protein, 533 aa, unidentified
  source 1..533: note = Uncultivated organism; MG119-165 effector
MNKNNDRKTQCIITRKFTLIPEVSDRKEWVKRVLNYVIEECDKKIEYFEKKQSGTSQNVNGKIKEKIQVLQLKKELAKSGKLEIDKKLAVDYTYDTVRRSMKSEAARKNYILSWAFSEMKGNGVSYMEPKERKKFIEELLKPAYRKKGSKLGSLFDDTEIENILKSYGVAFSKSFTKQLRKDVANGLLDGEISLRTYKSNSPFTIAKEHISLTHNYETYEELRENIGKKDCKLYFNFGGNGIPATLRFLINTGTAKNKDELNTTLLKLYSGEYELSGSSIQLDKTGKKIILNLSMIIPMKYHELDENTVVGVDLGVAIPAVCALNNDMYKREVMGSADDFLRVRMKIQAQRRRLQSSLKYNSGGHGRKKKLKALERLSKSEIHFVETYNHMVSRRVVDFAIKNNAKYINIENLKGYNTSQYILRNWSFYQLQQYITYKAEQHGIEVRKINPCYTSQVCSVCGHWEEGQRKSQAVFECGNPDCESHSKYKNGFNADFNAARNIAKSTLFMGDGEVTEKSKQEAREYCGIYREED

SEQ ID NO: 504 - protein, 525 aa, unidentified
  source 1..525: note = Uncultivated organism; MG119-166 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 505 - protein, 565 aa, unidentified
  source 1..565: note = Uncultivated organism; MG119-167 effector
MKTNKDETTITITRKYAIIPTSSETKEWNKRIYAFTADKLNKKITSLTEDIEEINDKPEKKKTDEDRKHLEELNKSLSDANSTLEEITKTKEYTQKMINDYTYSLVRTAMEEEARRKNYILSWMFSEMTKHGVQYMETLKEKQKFVKDTINYAYRKKGSKAGSLFDNTEITNILNGYGTAFKQAFTGKINDAVKDGLLEGKVSLPSYKIDSPFTLEKTTMGFTHDYENYEELCEHVNDKDLKLYFDYGGNQNPTIARFKINLGHGKNKDELKATLLKLYSGEYQYCGSSIQISKNKIILNLSMKIPKKEMDLDENVVVGVDLGMEVPAMCALNNSIYEKQPIGSADDFLAIRTRLQAQRRKLQIALKSTAGGHGRKKKLKALNRLRDREKHFVESYCHFVSKQVVDFALKHRAKYINIENLTGYDTSKFILRNWSYYKLQQYITYKAARYGIIVRKINPCYTSQVCSYCGHWEPKQRKSQDTFICGSEDCISNNKKKVKYTVNADFNAARNIAMSTLFMETGEVTEKTKEKAREYYGFEEEYQKYKAKNEEAKENKEEELYDLAA

SEQ ID NO: 506 - protein, 539 aa, unidentified
  source 1..539: note = Uncultivated organism; MG119-168 effector
MDSNNTQIVTRKLVIRPTFSERKEWKKKVTEFTLKDLEEQISKYKEWIENLKSNKKIDKEEKERRKQKYTDLLTNAENSLNQFKDREEITQKMVNRYTETLVKESMESEARRKNYILTWAYFSMRANGVQHMQPKEQKKFIREMLKPAYRVKNSSKGSIFDDVEIDNILGGYGMAFNQELTNKIIDSVEKGLFEGGKDSLPSYKYDSPFTVAKSTMGFSHDYDSFEELCEHIKEKDCNLYFDFGGNSDATIARFIIDVGTNRNKDELLTTLCRVYSGEYEYCGSNIQFDKTGKKIILNLTIKIPQKNCDLNENTVVGVDLGIKIPAVCALNTNPYAREFIGSVDEYLRVRTQIQNQRRRLSIQLRNTKGGHGRKKKLKALDRYTSRERNFVKNYNHMVSSRIIKFALKYNAKYINLECLKGYDADKKALRNWSYYELQSMIEYKANKYGIVVRKINPCYTSQVCSECGHYEEGQRVSQEKFICGNDDCRFHNTKSGYINADFNAARNIAKSELFLDSEVTKKRIEEAAKYYNIPTKDEV

SEQ ID NO: 507 - protein, 538 aa, unidentified
  source 1..538: note = Uncultivated organism; MG119-169 effector
MIKKQDNEIETIIITRKLNIIPIKSENNTCIKKVYKFHIDKINKKIEALVECESKEKDKKKEEKITDLSTDEKNKLEYFLNGEELTRPMVNDYTYTLVRDAMESEARRKNYILSYAFAKMTEARVDLMDIKERNKFIGQLLNPAYRAKGSKKGSIFDETEIDNILGGYGTGFSQELTSKIKDCVKDGLLDGKVSLPSYKLDSPFTIVKDHMGFTHGYDSYEELCEHINDKNCELYFNYGRHHEPTIAKFRINVGHKGNREELLTTLRRLYSGEYQHCGSSIQFDKTGRKIILNLSMKIPKIHMELDENTVVGVDLGIAIPAMCALNNNMYKREAIGSAEDFLRIRTKIQKQRRHVQTSIKNAKGGHGRKKKLKALTRYRQYEKHFVETYNHMVSSRVVKFALSNRAKYINIENLTGYDTSKFILRNWSFYQLQQYITYKAAKYGIEVRKVNPCYTSQVCSVCGNWEEGQRVTQSKFECGNKDCKSHDFKHGFNADFNAARNIAKSTLFMKDGQVTEKSKAEARKYYSIPENIEENKED

SEQ ID NO: 508 - protein, 552 aa, unidentified
  source 1..552: note = Uncultivated organism; MG119-170 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTINYAYRKQGSNKGSLFDDTEISSILKSYGIAWSQELTKEIKNLVKNGALEGKVSLTNYKLDSPFTIAKAHFSFDHDYDSFEELCEHIDDSDCKMYMNYGGDNKKDGTNPASLARFRINLGQGKNRNELKATLLKVYSGEYQYCGSSIMISKNKIILNLSMKIPKIETELDENTTVGVDLGIAVPAMCALNNDMYKRLAIGSADDFIRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAERYGIVVRKINPCYTSQICSVCGNWEPDQRKTQAKFECANEECASHDKYKYGFNADFNAARNIAMSTLFMETGEVTEKKKEEARKYYGIEEKYQRYQASLKEKDDKVA

SEQ ID NO: 509 - protein, 568 aa, unidentified
  source 1..568: note = Uncultivated organism; MG119-171 effector
MGTELQNSTIMKLNIKNKVDRESTKMELKNTQIVTRKLVIKPTFSYRKEWKKKVSDFTIKDLEDRILKYKEWIENLKVNKKLKKDEKERRKQKYTDLLRDAEKTLEEFKSGGEITQKMVNRYTESLVKDSMESEARRKNYILTWAFYNMLANGVQYMEFPEQKKFIQEMLKPAYRKKNSSKGSIFDDVEIDNILNGYGIAFSQELTSIIVEGVKNGLFSGSKETPPRYKYDSPFTVAKSTMGFSHDYDSYEELCEHINDKDCKLYFDFGGNQNPTIARFIIDLGTNGNKCELTTTLLRVYSGEYKYCGSSIQFDKTGKKIVLNLSLEIPQKECSLNENIVVGVDLGIKVPAVCALNVNPYAREYIGSVDDFLKIRTQIQNQRRNLSKQLKNTTGGHGRTKKLKALDRYTKRERNFVKNYNHMVSSRIIKFALKNNAKYINIECLKGYDTDKKILRNWSYYELQQMITYKANKYGIVVRKINPCFTSQVCSECGNYEEGQRISQEKFMCKSEKCRFHNLKKGYINADFNAARNIAKSELFLDSEVTKKKIEEAARYYNIPYEFKNTDEV

SEQ ID NO: 510 - protein, 535 aa, unidentified
  source 1..535: note = Uncultivated organism; MG119-172 effector
MAAKDNTMVITRKIALIPVVSERKEWKKRINAFLEKDFPRKIEAKKKQIKNTSKPENKDGYKLQLAELEKQYEDFKENRIEEYTQKMVNDYTYGLVRDAMESEARRKNYILSYIYTKMVQDGVGYMPTLKDKYTWIKENINVAYRRKGSNKGSIFDDVDIDNPLGGYGTCFSQILTSKIKDLVKQGLLDGKVSIPSFKIDSPFTIAKAYFDLYHEYEDIAELKKRIYDSDCKLYCGLGNKGIPTIANFKVNIGHKGNRDELISTLLRIFTGEYEICGSTMQISKTKIILNLSIRIPKTQLELDEDTVVGVDLVVAIPAVCSLNNNKYKREYIGSSDDFIRMRTKLQHEKQQLQKALKMSKGGHGRKRKLLALERCKKKEHNFVETYCHKVSKKVVDFAVKNKAKYINVENLSGYDSSKFILRNWSFYKLQQYIEYKAAKYGIEVRKINPSFTSQVCSFCGHWEDGQRKDQATFVCGNPNCESHKLHMVNADYNASRNIAMSTLFTSDKFQFCKKTMQNAAEYYGIQLDNEDQLAA

SEQ ID NO: 511 - protein, 521 aa, unidentified
  source 1..521: note = Uncultivated organism; MG119-173 effector
MAKSSENVLTRKVQLIPEISDRKKYTKRLTKYINDSYSEKIDKKRNKKFNRKDYDSLEKFKEAKEKNESEIQNLQDALANLAAGTITKKMVDDYTYGLVREAMESESSRKNYILSHVYSQMVTSDYAHMDKEEREQFFKDIWNPCFRKKGSNLGSIFDNTDIDNILGGYGTGFSQELKGKINNAIKSGLLEGKVSLPMYKKDSPFSIESQYLKFYHAYESFEELENNIEKPNCQLFMGFGKNGNSTIANFKVNLGCGSGAKKRKDNSICQTMLKVLAGEYKICGSSIQISKGKIMLNLSIDIPIKEHKLDPNICLGVDVGYVIPAMCGLNKGKLTFRDARFGDGTDLNGRRKRIREQKKRMQVGIATNKGGHGRAKKIKALDRFREKERNFARTYNHKVSHDIVNLALKNNAMYINLENLKGITSDEKKKRELGDWSYFQLQEFITYKAKQHGIIVRKVNPYRTSQTCSCCGHWEEGQRVDRDTFICKNPECKWFGKKIHADLNAAINIAKSTDFSVKKDA

SEQ ID NO: 512 - protein, 532 aa, unidentified
  source 1..532: note = Uncultivated organism; MG119-174 effector
MANKSSKKPLIITRKYALIPIESPSPIWTKSVISFLNKDYISKIEYKKQLIEIENKKGKKKDNNKIEKWEKEIANIESKIELVDNGDFTSGIVNDYTYNLIRKCCEEQSIIKNHTMFAMGAAYKDCIIKGIDEKERTEIVTDVFNSWSRVPNSSKGSMLDKMNIDLSIGSYELAYVNALKNKFWECVKDGFAYARQTLPYYKSDCPMDIASKEMSFTHDYESFEELCEHINEKPNLYFNYGGNGKPHIFRFKINTGHGKNNDELMATLMKVYAKEYKVCGSSIQIQKSGNDKKDKIILNLSLEIPKVKRELDKNICVGVDLGIAIPAMCALNTNDYVRQSIGSKDDFLRVRTKISNQRSRLQASLKMSNGGHGRKKKMKPMDRFEDYEANWVQNYNHFVSKQVVDFAIKNKAKYINIENLEGFDANNYLLAKWSYYQLQQYISYKAKINGIEVRKINPYHTSQRCSCCGYEDKGNRPKGKKKQAYFKCLKCGKEMNADFNAARNIAMSTEWSDGKTTKEQKKKQHEEYIKKE

SEQ ID NO: 513 - protein, 525 aa, unidentified
  source 1..525: note = Uncultivated organism; MG119-175 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTKYKKGFTADFNHARNVAMSTLFMENGSFSKKSMDEAAEYYGISEKYKA

SEQ ID NO: 514 - protein, 525 aa, unidentified
  source 1..525: note = Uncultivated organism; MG119-176 effector
MAEKMTMITKKCVLIPERSDSSLWERRVYDFTVKDYEQRISFYESQLESKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 515 - protein, 579 aa, unidentified
  source 1..579: note = Uncultivated organism; MG119-177 effector
MKKNNKESNTYIKAMKIRIVPICDIEEENLRDKYYKELYQYLRDSIWAQNAALNYGLAMTRDAYVLHKNEDKIKDIYFKLAHQTPNPSMASEARLKEVYEAAPITQEYIDNKVEEFKKSNSKKKKPLTDEGVKKKTDSINNMYKKFIGLSKEDIQELIDKLDDYCAYPEEIYEKFANGFSTPAYVTQQIKDYWNTEGVMTKVIYSMSDKLRSVKNSNPLTIPPNLFYNKKNDLIGITHSYNTYLEFVEALMNVYDANLFLELPYKKGYDKMKFKLILGNPCKSHEVRISLQRIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGEGYTLNADYNAARNIAMCDLFMDNKSEITEEDKRIAREKYGIPEPIKKKEKLAA

SEQ ID NO: 516 - protein, 571 aa, unidentified
  source 1..571: note = Uncultivated organism; MG119-178 effector
MVTSNTTANVRKMCKTVKIKLIPMGTKKGTAKQERERVIQYLKDSEKAQQIALNHIIGQLTAEYYACGSNLNSPEFKERRKEILNYWNPVFHDVVYLPPSSELKSAITWKLNKEFSSIIKNGLAKGERTVTNYSLHCPIPTAKNRINFEHNCSSDIEFLEKMYTKEVDVQMRWINRFRFRVDLGNLSKSKKLRKTFAQLFLGNIEKCDSSIQLCKNKEIFLNLCIMVSETLVPLNEETVVGVDLNLNHPAVCALNNDKTKMLFVGNIAFRGTRETLKAARLSKQSSLMTANGSHGRGKKLAALKSIKRRERNFAKTYNHTVARRVVNFALENNAKYINIENLQGATTQIQKDSSMEFVSSNWSYYELQQYIKYKAEAVGIEVREVNSYNTSKMCSYCGSENDEQLVSFHEFVCSNPNCESHTLFPKAMDADFNAARNIALSTEWSSGKKKFADSKKQHEDAKSEKEKSEEEKETKEKSNTAAKKAKSTDPYDFETLKANGILSARTANALTTAGYKNVSDLHGKSVKDIKKIRGFGDSAINQLFKYLNSLDFVSVPANPSEKLSVTDAPRG

SEQ ID NO: 517 - protein, 525 aa, unidentified
  source 1..525: note = Uncultivated organism; MG119-179 effector
MSEKMTMITKKCVLIPERSDSSLWERRVYDFTVKDYEQRISFYESQLKSKNKEVKRNAKTRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTKYKKGFTADFNHARNVAMSTLFMENGSFSKKTMDRAAEYYGISEKYKA

SEQ ID NO: 518 - protein, 525 aa, unidentified
  source 1..525: note = Uncultivated organism; MG119-180 effector
MAEKMTMITKKCVLIPERSDSLLWERRVYDFTVKDYEQRISFYESQLESKNKEVKRNAKIRLEILNMELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNQTLHIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKTMDRAAEYYGISEKYKA

SEQ ID NO: 519 - protein, 525 aa, unidentified
  source 1..525: note = Uncultivated organism; MG119-181 effector
MAEKMTIITKKCVLIPERSDSSLWERRVYDFTVKDYEQRISFYESQLKSKNKEVKRNAKTRLEILNKELDDFLDNPETTQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKNTVVGVALGLVQPAVCALNNDQYVRLPIGDERDLLDNKLQIQERSRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 520 - protein, 525 aa, unidentified
  source 1..525: note = Uncultivated organism; MG119-182 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKNHTNYKKGFTADFNHARNVAMSTLFMENGSFSKKSMDEAAEYYGISEKYKA

SEQ ID NO: 521 - protein, 549 aa, unidentified
  source 1..549: note = Uncultivated organism; MG119-183 effector
MINKDKNENTVTITRKYALIPVSSDKTQWYKKVYDFTIEDLTRKIEYYKEKLKKEKDKEKKEQIKTKIKNFEEGLESIYNGGDFTQKMINDYTYNLVRTAMEEEARRKNYILSWIFSEMRLNRVDQMESLKDKFKFISDTLNYAYRKKGSNKGSLFDDTEINSILNAYGIAWNQELTKEIKDLVKNGALEGKVSLTNYKLDSPFTIAKAHFSFDHDYDSFEELCEHIDDSDCKMYMNYGGDNKKDGTNPASLARFRINLGQGKNRNELKSTLLKVYSGEYQYCGSSIQISKNKIILNLSMKIPKIETELDENTVCGVDLGIAVPAMCALNNDMYKRLAIGSADDFLRVRTKHQEQRRRLQKSLKNSNGGHGRKKKLKPLERMNKAEAHFVETYCHMVSKRVVEFAVKNRAKYINIENLNGYDSSEFILRNWSFYKLQQYITYKAARYGIVVRKINPCYTSQICSVCGNWEPDQRKTQAKFECANEECASYDKYKYGFNADFNAARNIAMSTLFMETGEVTEKKKEEARKYYGIEEKYQASLKEKDDKVA

SEQ ID NO: 522 - protein, 452 aa, unidentified
  source 1..452: note = Uncultivated organism; MG119-184 effector
MIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGTAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMERLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 523 - protein, 358 aa, unidentified
  source 1..358: note = Uncultivated organism; MG119-185 effector
MLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQILKNKIVLNLSLQIPKEEKDELDKDTVVGVALGLVQPAVCALNNDQYVRLPIGDERDLLDNKLQIQERRRRLQSALRETSGGHGRKKKMQALNRIAKTENNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKTMDRAAEYYGISEKYKA

SEQ ID NO: 524 - protein, 448 aa, unidentified
  source 1..448: note = Uncultivated organism; MG119-186 effector
MTNTYIIARKIQLIPVGKKEEVNRVYTYIKDGIKSQNMAMNQYMSALYTSTIMEMTKDDRKELSRLFGRISTSKLGSAYDKTIEFAKGLPMGGAIQQKVKKDFDNAMKKGLKYGKLSLPSYRDSNPLLVHVDYVRLRSTNPHLDNGLYHNYSSHEEFLNHLYNEKLELFIKFANKITFKLVLGNPRKSAEIRSVFKNIFEDYYHIKGSSIELDGTKIILNMSIEIPKKKVVLDENVVVGVDLGIAIPAVCALNTNDYVRKSIGSRDDFLRVRTQIKSQRDRLKRKLKYTRGGHGRAKKMQAMDRFTDYEKNWVQTYNHKVSKDVVDFALKNKAKYINLECLDGFSKDDKKKNYLLANWSYYQLQQYIKYKAEKYGIVVRFVNPAYTSQTCNCCGNLEEGQRVKQSEFICKNPECKNFGKKINADFNAARNISMSTDFVEKEEEKERIT

SEQ ID NO: 525 - protein, 474 aa, unidentified
  source 1..474: note = Uncultivated organism; MG119-187 effector
MANKYIISRKITLIPAAPLNNQWQKRFDKYVNLSFERVQEQIIEAQAELDDEETKKTRKDSLSKWIDSLYKKADMYSDYISEGEITKALLNDYVYTFVRNACKAESRHKNLVISKVIDTMRINGFYDLKSDEARELYRSTFNAAVRLNNGSFSDIINNPCGGYGVSWSNALNASLKREIINGCISCRNGQVKNFKEMSPFSIAKSAMGFSHGYDSDEIMLANMDEKNAPLYFDFGGNGTPSLLQFRMNLGANPKNKNELVSTIKRVYTGEYQYCGSSIQISGNKIILNLTLEIPKPADMLESLSKNTVLGLSLGTNTAIIGALNDSKSYFTFGTGHELEYYRRYIQESYRNLAKAVKYTNGGHGREKKLKKLEVFKVRERNFVKSFNHKASRTAVDFAVKHNAGLIRMPDLSEFSSKVNDDESKYLLRNWSYYQLQQMIEDKAARVGITIEYVPIPQVEDGVADIDIARKIANN

SEQ ID NO: 526 - protein, 433 aa, unidentified
  source 1..433: note = Uncultivated organism; MG119-188 effector
MILTRKIQLLIVGNNDEVNRVFNYIRKGMISQNKAMNQYMSALYLAELNKVSKEDRKELNQLYSRISNSKKGSAYSQDIVFPKGLSVASDLSMKVKQDFKQSCKNGLMYGKVSLPTYRSNNPLLIHVDYVRLRSSNPHRDSGLYHNYKNHAEFLEHLDNKDLEVFIKFANNITFKLIFGNVKKSASLRHEIQMVFEEYYKVCSSSIEIDGRKIILNLSMDVPEQKIELDENVIVGVDVGIAIPAVCGLNTNDYSRKYIGSVDDFMRVKTKIQHQKSRLQTNLKMAKGGHGKKRKLKAMDKFTDYEKNWVQSYNHYVSKQIIDFALKNKAKYINIEDLSGITKGKNVNKFLKGWSYYQLQSFIIYKANKYGIEVRKIDPHYTSQTCSCCGYVDEKNRPKNEKGQSYFRCLKCGHEENADFNAAKNIAKSVNFVK

SEQ ID NO: 527 - protein, 470 aa, unidentified
  source 1..470: note = Uncultivated organism; MG119-189 effector
MIMVKKIAIIPAASDNAMWLKKFNEYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSGNKVGVSLGLNTAVIAVTNANKKDVLKFGDEHTLEYYRVYIQNRLWELKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKTLSEDIDGVADIDKARMMLE

SEQ ID NO: 528 - protein, 458 aa, unidentified
  source 1..458: note = Uncultivated organism; MG119-190 effector
MEKNTQIITRKYNIVPCKADTKQWYEKCYEYTISDINAKIDRAEYVLKKKDGIENRDRLQEKIGQYRKMLDEMEKHKEDFDFTNQMIDTYTKDLVREAMESEARRRNYILSWAFATMTENGLQHETDFKARNKFISEMLKPAYRVKGSNKGSLFDDTEIENILGGYGTSFNQELTRKIKDSVNKGLLEGRVSLPSYKMDSPCSIAKAHMGFSHDYSSYEELMAHIEDRELKLYFDFGSPDKDAGMAKPTIARFRFNLGGCRNRQELLTTLLRVYSGEYQYCGSSIQFNKQGNKVMLNLSMEIPVRENILDPDTVVDIDLGVDIPAICTVENSGSTLDIGSKEDLLRIRTQLQIQRGNTQRNLVIAKGGHGRKKKLRKLETLKAREANFANTYLHMVSRKAVDFAVENHAGCIRLKNIEEYDSQDYLLKNMSYYKLCQQIEYKAAIQGISVVYAEKERK

SEQ ID NO: 529 - protein, 390 aa, unidentified
  source 1..390: note = Uncultivated organism; MG119-191 effector
MSEKRMTITKKMQLLVDGDKDEVKRVYDFIRNGQYAQYRAMNRYMGEIASIYYKHNMDFKSDGFQTEYKEIRTNKNEIFQDLEFSTGVDTISQARRTVESDFMASVKNGLAKGERTITNYKREYPLLTYGRSLKFYHDYADDYEMYDNLDNKDFLIYLGWVNKIRFKVRFGNPYKSHELRTSVARICDGTYKIQGSKITIIGKKIFLFASVSIPILEKEMLEDNVCGVKFGKCVTATCVTNIDKKKYVECGNADVFLMQRAKMKAMRERTMAQLKYSKGGHGRKKKLKPLERFRKREHNFAKTYNHTISKEIVNFAFDNRCSKIVLEDLSIYENDDEFVTKNWSYFDLEEIIKYKAKRYGITVETKKLDIEKVKDENWSNVDLAFELTLD

SEQ ID NO: 530 - protein, 357 aa, unidentified
  source 1..357: note = Uncultivated organism; MG119-192 effector
MAKKQNKNTMQINRKITLIPVASDNDSWKKKMNTYLEKFYFDKKIKAKERQIKNTSKPERKDEYKQQLAEYKKQQEQFLNGELEDYTKQMVMSYTYDLVRDCMESEARQKNFIMSYMFSEMIREKVCYLKNKKEKEKWVNDNINVAFRVKGSPKGSIFDDVEIYSPLRALSIQGYTQELKGRVKKFATDGGLDGKLAVDNYKLDSPFHMSKQGFDIVHEYEDLTELKKNIGKSSCEIYVNLGNGGVPTFARFQLDFGHKGNREELISTITKVFTGEYKVCGSSIQINKKNKIILNLGLEIPKRLTELDENTVVGVDLGLAVPAVCSLNNNQYKKEYIGDGEAFVKQRGKIQKEKQRL

SEQ ID NO: 531 - protein, 419 aa, unidentified
  source 1..419: note = Uncultivated organism; MG119-193 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTNPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGEHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 532 - protein, 419 aa, unidentified
  source 1..419: note = Uncultivated organism; MG119-194 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 533 - protein, 460 aa, unidentified
  source 1..460: note = Uncultivated organism; MG119-195 effector
MKTEETIDWIDSCIVCAGRQERRKEGMLPGMFGSLILTRKIQLIPCTEGMTTDEAKVEVNRIYQLLRDGIYAQNKAYNIFISRMYTATLLGASKEELKEIRLKGERTPKESDSNYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 534 - protein, 457 aa, unidentified
  source 1..457: note = Uncultivated organism; MG119-196 effector
MTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYHDFLDKINDSDLAVYVKWVNKIVFKVVFGNPHRSLELRSVIQNILEENYKVQGSSIEIDGKSIILNLSISIPKQLRELDENIVVGVDLGIAVPAMCALNNNLYERLAIGNADDFLRIRTKMQAQRKRLQKSLRNTSGGHGRAKKLKALERLQKAEVHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKTGFNADFNAARNIAMSTLWMGSGQVTEKSKQEAREYYDISEKYEQSKNGSENNKVA

SEQ ID NO: 535 - protein, 426 aa, unidentified
  source 1..426: note = Uncultivated organism; MG119-197 effector
MILTRKIQLIPCTEGMTTDETKVEVNRIYHLLRDGIYAQNKAYNIFISRMYTATILGASKEELKEIRLKGERTPKESDSDYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 536 - protein, 412 aa, unidentified
  source 1..412: note = Uncultivated organism; MG119-198 effector
MGTFCKKIRLYPIGDQEEVNRVYEYIRNGMYVQYTLMNKLMSQIMTLYYSDEVDRDVKSDAFKNGRREILRISNPVFHDEPMPKGLGMQSTVTRKVSQDFSTALKNGLASGDRSAPYYRRSCGLMVPGVRFQFYMSDDTYCIKFCNGIHFKVITGRGKRDYYLTDLLDALSSGNEDYHIGTSQLTIDGKYIYLILTVSKEDVEVSYEPDKDRVMGIAFGYESPTVCCIRNLTKPQVSYIEIGDSTPLVKDRMRIQENMRMLTTGLKNCTGGRGRKHKLQALDRYTKRERNFVRTYNHKLSKDIVSAALDAQVSDIVMEKVEKQDVSAVMLRNWSWYELQSMVSYKAAKYGITVSVDDRKIGLVCSQCGCVQSEQADSEFNTSGTFFCTECGHRMPAGQNKALILVSDTKEKA

SEQ ID NO: 537 - protein, 419 aa, unidentified
  source 1..419: note = Uncultivated organism; MG119-199 effector
MANVLTRKIQLVPVGDKEEINRVYNYIREGQYSQYLGLNRLMSELVVTFYKNDRKLDEKFNEERKNILSNSNTILNDILFPKGCDTKSHIAQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNSNCKNYGKEINADFNAARNIAQCSDFIK

SEQ ID NO: 538 - protein, 474 aa, unidentified
  source 1..474: note = Uncultivated organism; MG119-200 effector
MENKMIMVKKIAIIPAASDNAMWLKKFNEYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSENKVGVSLGLNTAVIAVTNANKKDVLKFGDEHTLEYYRVYIQNRLWRLKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKPLSESIDGVADIDKARMMLE

SEQ ID NO: 539 - protein, 471 aa, unidentified
  source 1..471: note = Uncultivated organism; MG119-201 effector
MILTRKIQLLPLGDKEELNRIYTYIRDGMFNQNKGMNQFISALYVSFINEFAKEDRKELVRLYGRNPESSKGSAYSADMSFAKGLGSASSLTRKCQQSFDKAVKDGLLYGRVSLPTFRMDNPLLVPGRFIDVKGNRIKNDGHTETRGIYHLYASHEEFLDHLHNNDLEIYLDFVNGIHFKFVLGNPHKSRFLREEIKQIFEGYYKICDSSIGIDGKKIMLNLCMDVPKKEIVLDENKVIGVDMGLAVPAVCVCYNRSTNKYEGTPLYIGEIDELLTVRTRLQAERRRLQKALADCTSGGHGRKKKLAKLEDLKKRERHFVQTYNHMVSRRIVKFAEEHGAKYINVEDLSDLDSKLKGKLKDKFDEESGQFVLRNFSYFERQNYTEYKAARYEMVVRKINPDKTSQTCSCCGNYEPGQRISQSAFKCKKCGYEENADVNAAKNIAFSENFVEKVKTPKKTKKNEQLDGIEND

SEQ ID NO: 540 - protein, 459 aa, unidentified
  source 1..459: note = Uncultivated organism; MG119-202 effector
MEIKANRMIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGTAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMECLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 541 - protein, 401 aa, unidentified
  source 1..401: note = Uncultivated organism; MG119-203 effector
MYLVRKIRLIVIGENKEDVNEKYKFIRDAQYAQYRGLNYMMGQVGALYYSCDRNISSDEFKTQYMEIFRASNQVVDGIEWGTGIDTRSLVGTRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 542 - protein, 459 aa, unidentified
  source 1..459: note = Uncultivated organism; MG119-204 effector
MEIKANRMIITKKLKVIPDGDKEEVKRVYKYIRDAQYNQYRYMNRLMSNVCMIYYKYDMDITNPNLKEEMKELYRVSNAAWIKEDNPIGSGMQATATSRVKQDFSTALKNGLAKGERRLPQYKRDFPFLMNGAAIKPFIKNVTYTDTNSGEDKIRTEYHLKWVNKISFRIWLPTNAYKDLNIQTIIAALNAVIENKEGYKICGSSIRVEDKDIFIFLTLDVPSKKTSYTPVTGRTMGVALGYDSPLVCCITENDKDSKFFAVGENLDFVENRKKRHEKYRQIQSATKECHGGNGRKRKMERLNAAKSNESNYRKDINHKLSKEVIDLATKNKVANIIIESTTADELKTVPDMLRNWSYYDLVAKITYKGNMYGILTEEMKKTEKKSTKKIKRADCCPACGEVFASGNANALTPEDYWNTSTEFVCPSCGTVEEYGKVRAFSSACKAMLPEEKVKNNKTE

SEQ ID NO: 543 - protein, 426 aa, unidentified
  source 1..426: note = Uncultivated organism; MG119-205 effector
MILTRKIQLIPCTEGMTTDEAKVEVNRIYQLLRDGIYAQNKAYNIFISRMYTAILLGASKEELKEIRLKGERTPKESDSDYSLYDFDKIKFIKGLPQASALGEQALKALKKQRKDGLYKGKVSLACRKLDAPMWIKTKFSFFHKYNDYQEFLDHLYCDDLKIYMKFVQGIVFEVVLGNPHKSETIRTEFQQIFEGHYKLCSSSLQIKDKKIMLNLAIDIPEKGIELNDETVVGVDIGIAIPAVCALNNREYVHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 544 - protein, 426 aa, unidentified
  source 1..426: note = Uncultivated organism; MG119-206 effector
MILTRKIQLIPCTEGMTTDEAKVEVNRIYHLLRDGIYAQNKAYNIFISRMYTATILGASKEELKEIRLKGERTPKESDSDYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 545 - protein, 452 aa, unidentified
  source 1..452: note = Uncultivated organism; MG119-207 effector
MAKDRVTICKTIKLYPVGDKSEVDRVYKFIRDGQYAQYRASNRLAGELYSEYYRFNMDIKNEEFKTRQKEIMRNSNPLFDDIEFAIGVDTPSAVTQKVKSDFSIALKNGLARGERNTTNYKRTNPLITRGRSLKFVHEYENYQEFLDHINSSDFAVFIKWVNKIKFKIVFGNPHRSQELRGIVKNIFEDNYKVQGSSIQVDGNEIKLNLSLSIPKEIKELDENTVVGVDLGIAIPAMCALNNNLYERESIGKVDDFTRVRQKYQAQVKRLQKSHKSATGSHGRSKKMRALNRVKKSEAHFVETYCHYVSKKVVDFALKHNAKYINMENLKGYDTSRFILRNWSYYKLQQYITYKADRYGIVVRKINPCYTSQVCSVCGHWEPDQRKTQASFECANPGCESHKKYKYGFNADFNAARNIAMSTLFMQDEEVTEKKKEEACEYYGIKKENSEAV

SEQ ID NO: 546 - protein, 462 aa, unidentified
  source 1..462: note = Uncultivated organism; MG119-208 effector
MGNDRMTICRKIKLFPVGDKEEINRVYNFIRNGQYAQYQACNLLMGQLMSEYYKYNRDIKNEEFKARQKEIMTNSNIILKDIDFATGVDTPSAVTQKVKQDFNTALKNGLARGERTVTNYKRTNPLITRGRNLTFYHEYETYQDFLDKINDSDLTVYVKWVNKIVFKVVFGNPHRSLELRSVIQNILEENYKVQGSSIEIDGKSIILNLSISIPKQIRELDENTVVGVDLGVAVPAMCALNNNLYERLAIGDADDFLRIRTKMQAQRRRLQKLLRNTSGGHGRTKKLKALDRLQKSEAHFVETYCHMISKRVVDFALKHNAKYINIENLTGYNTSDFILRNWSYYKLQDYITYKAAKYGIEVRKINPCYTSQICSVCGNWEFGQRKSQSVFECANEDCDSHKKYEKTGFNADFNAARNIAMSTLWMENGQVTEKSKQEAREYYGISEKYEQSKNDSENNKVA

SEQ ID NO: 547 - protein, 474 aa, unidentified
  source 1..474: note = Uncultivated organism; MG119-209 effector
MENKMIMVKKIAIIPAASDNAMWLKKFNKYVEFSRRDAEEKYKAVSEKKNLKDKDNILKNLRNKMDRADKFLADGEITKPLLNDYVFNFCRDASREEAKHKNYVISKVIDTLRIAGYEDLSSDEAKKIYKDTFNAALRTKNSTSAAFINNPLKGYGLSWSQTLNSSLKKDIFNGAISCKNGQARNFKPDSPFTVAKSMMGFSHTYDSDGEMYAHISDTNANIYFDLGAKGKPTILRFKLNLGANPKNKNELISVIKKVYSGEYEYCASSIKIEGAKILLYLCLAIPEDKEELKSENKVGVSLGLNTAVIAVTNANKKDVLKFGDEHTLEYYRVYIQNRLWRLKKDLKYINGGHGRKEKLEKLNIYKDRERNFVKDFNHKASFAIIKFALKYQASVIVMPDLTEFAQRLTKEIKEARKNHETYSHKYMLRNWSYYELTSMVEYKAARKGITVIYKPLSESIDGVADIDKARMMLE

SEQ ID NO: 548 - protein, 466 aa, unidentified
  source 1..466: note = Uncultivated organism; MG119-210 effector
MILKSHVNGLQIPINTRAANILRENTRMSKRTTMIQRVSLIPIGEKEQTDKAYKYISDSIYAFYKTNNQIMGEIYSAFYNNGCNLNLEEYKAEHSELFSYDNPAISKIEYPGGIDILELSIKKVESDFKKALLNGLAQGKITAKNYKRTEPLPVSKETLNFDIKFENLKNGTPNIRSLSATLNWVNGISFEVYFEKSGYGFNTQNSFYKIHTGEYTIEDSKIIIEKNKRGIRGINLYLSLNVPGKVHTLNEDTVIGVNLGLSRTIVCCLNTDNYKRLYLEPDLLIKSTEQRIYSAEKDVKKLSLSTRTEHKRQAKEQFVNRLKHCKESLIKRINDKATTNILLFAQKNNAKYINIENMAWYGAPQKNSLLVRNGNYADFEEQLIRKSHKFGMIVRKINPYSTMHTCSFCGCQEDGQLISDTRFLCKNPDCVSHTFKGKGEANKKYIEPDFNAARNIAKSTEFKKSH

SEQ ID NO: 549 - protein, 466 aa, unidentified
  source 1..466: note = Uncultivated organism; MG119-211 effector
MILKSHVNGLQIPINTRAANILRENTRMSKRTTMIQRVSLIPIGEKEQTDKAYKYISDSIYAFYKTNNQIMGEIYSAFYNNGCNLNLEEYKAEHSELFSYDNPAISKIEYPGGIDILELSIKKVESDFKKALLNGLAQGKITAKNYKRTEPLPVSKETLNFDIKFENLKNGSPNIRSLSATLNWVNGISFEVYFEKSGYGFNTQNSFYKIHTGEYTIEDSKIIIEKNKRGIRGINLYLSLNVPGKVHTLNEDTVIGVNLGLSRTIVCCLNTDNYKRLYLEPDLLIKSTEQRIYSAEKDVKKLSLSTRTEHKRQAKEQFVNRLKHCKESLIKRINDKATTNILLFAQKNNAKYINIENMAWYGAPQKNSLLVRNGNYADFEEQLIRKSHKFGMIVRKINPYSTMHTCSFCGCQEDGQLISDTRFLCKNPDCVSHTFKGKGEANKKYIEPDFNAARNIAKSTEFKKSH

SEQ ID NO: 550 - protein, 442 aa, unidentified
  source 1..442: note = Uncultivated organism; MG119-212 effector
MPNITRKYQLKVIGDKEEIDRVYKYIREGTEAQNKALNEAMSALYAANLLDMSKDDKKELSKLFSRVTNGKNESGFTNDICFATGLGTTANIKQKVKQDFNNACKKGLMYGRVSLPSYKTDNPLLVSKSYVQLLSEGDKNFGIYSAYETPMDLVDALEKETNPEVYLKFANNILFKFVFGNPWKGREQRKVFERIFSGEYKICGSSIGIDGKKIILNLCMDIPKQEHNLDENIVVGVDLGLAIPAMCALNNDDYKRLSIGSIDDLLRVRIQLQNERKRIQGNLKNSKGGHGRQKKLKALENLKDRERNFVQTYNHMVSKRVVDFAVKNNARYINIEDLSGFGKTRYGKSKSDDEKVLRNWSYYELQNYITYKAQLHGITVKKVRAEYTSQTCSYCGSKGIRKEQKKFVCVNPDCKCHKIYDGYINADFNAARNIAMSNDFVE

SEQ ID NO: 551 - protein, 474 aa, unidentified
  source 1..474: note = Uncultivated organism; MG119-213 effector
MAITTKKIQLKIVGDNNEKNRVYTYLRDGIYNQHLVLNTYMSQVGTLYYSVNGDIKSVEWKEKYNEIFRNTNEQVKVIPYPKGLNMQANAGRKVQADFSTALKNGLASGERQLPYYKKDVPLIVPSRFLHFYSEEETYEKDGIKDTRNAYYIKFVNGIVFKVVIGPSAIKDFYFLPLLDGICNNPLVYDVRGSSIQIAKGNKIILNLTVAVNKHTEDYTPIKGRTMGVAFGYNRSLTVALSDDDNTYYIGDKVFGKNINEEIISKRIELQDYRTRLQKSVRGAKGGHGRTRKIEALDKFRRSEKNMVKHYNHILSKEVVNFAKNHNVEAIIFEDVDKDDLKDNPVMLRNWSYYELCNFIKYKAKSICKNEDNEIKVLMANANGIVEEKKKKKKKDKNASKEKPIEVKTEKNVKEDDTDKIRTKCHICGFAIRDKDRQKVSKVDEWISEQYFTCPNCDNLIEYSYNKAKNMSLLG

SEQ ID NO: 552 - protein, 456 aa, unidentified
  source 1..456: note = Uncultivated organism; MG119-214 effector
MPNLTRKYELQVVGDKEEINRVYTWLRQGIEAQNKAMNEYMSHLFMAKSCEMSLDDRKELNNLFQRVTNSKKGSGYTKDIEFPVGLPIAASVSRKVKQDFDNACKKGLMYGRISLPTYRSNNPLIIHVRFVALRGTKKVDNGIYHNYNSPADLFSALENDTKPEVFIKFCNNITFKFRFGNPYKAKEQREVFKKIFTEEYKVGGSTIEIKGTKIIINLTLEVPQIKKCSDENITVGVDLGQAIPAMCALNNDYYKRLAIGNVDDFLRIRTQMQEERRRLQCHLRNANGGHGRKKKLRALERLKQREKNFVQTYSHMVSRKVVDFAVKHNAKYINIEDLSGFGRDKYGHVKDDEQAKMVLRNWSYYDLQEKIKYKANIYGIEVRKIDPKFTSQICSVCGEKGIRQTQKEFICINPECSCHKMYQNQKYINADFNAARNIAMSQKFINEDKETTKKNE

SEQ ID NO: 553 - protein, 451 aa, unidentified
  source 1..451: note = Uncultivated organism; MG119-215 effector
MVTTKKIQLIPVGDPEEKKRVYEYLHDGIYNQCCILNTYMSQVATIYYKYNKDFKNADFKNEYNAIFRNTNTAIKDFQQAKGLGMAGNCGMRVKQDFSTALKNGLARGERSIPYYKRDFPLLCPSRFLTFYTGEDSFLNDKDEEVSVDAFFIKFVNGIHFKCVLGSKNKRRADLYLPELLSSIVEDPEHYHVCGSSIQFHGTKIILNLAVKIEKDAETYNPVEGRTMGLAMGYNKCLVAALSDSDRQYNIGDSNNPEYDLSRQIVTKRVEIQEHRTSLQKSLKGAAGGHGRSKKLKALDTQKKHENNVIRHFNHLMSKSVVDFAKKNKVEVIIMESIEKKDLDDVFLRNWSYYQLQSYITYKAEREGIRVIMSNATSASDKKKKGKKKDTTEETVMKTIHNTCCMCGKEIKREDILPEKIVWANAVSFTCPHCGNHLDYSFNKAKNMTTMG

SEQ ID NO: 554 - protein, 417 aa, unidentified
  source 1..417: note = Uncultivated organism; MG119-216 effector
MKNTFVTRYRVLPVGEKEERDRIYQYYRDAQYNQYLALNQAMSMAGIIYYNCHRNIKSEDFKTQYKDMFKRNSTVWDGVDFPKGGHLQGNVSRKVQSDFSTALKKGLAKGERQLPRYRRDFPYMIHGAEIKPYAQEEEYEGKTRTAYYIKSVNKIHLKFVFGSAERKQYRKVDMIKKLAEEDPAYKICQSNLFFKDNKMFLNLVVTKDVETKKYDPVKGRIMGVSYGFNCPAQTAFSDSENTYSLGDGDVFVEKRIQLQKNYNSLQKALRNAGGGKGRKKKLRALERYKAKEKNFAKQYNHKLSRQIVDMAKEKKVEKIILQQIDTKKLKKNPVLLRNWSYSQIYEMIKYKAEYGGIETDIRPAEDICPCCGCFIELPEFDNEMYEKEFVELCTECGEKINVSQNRAQYLAKQIGEN

SEQ ID NO: 555 - protein, 470 aa, unidentified
  source 1..470: note = Uncultivated organism; MG119-217 effector
MPTLTRKVELYVVGDKEEVSRVYDYIRLAMNATYKCFNECMTALYIAQVKEDTKEDRKELNYLYSRQTYTKKETAFTNDIVFPTGLVRASYINHEANLKFKTSMKNGLMYGCVSLPTYKKDCAVPLRCEFVSFAENKDKGLGFYHEYADVNELVNALEYDNNPKVFLKFPNNIRFEVVFGNPYRGREQRSVFSKIFLGEYKTCGSSIQINSKGRIILNLTMEVPKKEMEQIGGRVVGVDVGLAIPAMCAINDDNYARSAIGDINDFLRVRTQIQNQRRRLQKSLKNTSSGHGRAKKLKPLERISERERNFVNTYNHMVSKRVVDFAVKNGASQINIEDLSGFAKDKNGKSVEDANMKKILSNWSYFELQQQIRYKAGQYDIKVRTVNPAYTSQTCSYCGQIGKRETQSKFVCTNPDCSCHKMYKKDWFNADFNAARNIALSTDYTDDEDGKKTKKKKSAKKKLEKKTEEA

SEQ ID NO: 556 - protein, 453 aa, unidentified
  source 1..453: note = Uncultivated organism; MG119-218 effector
MLEAQTITRKIQIIPVGDKDEVNRVYTYLREGIKSQNMAMNQYMSALYSAMVADISKDDRKELNKLFSRISTSKLGSAYDETIEFAKGLPMGGAIQQKVKRDFDNAMKKGLKYGKLSLPTYRDNNPLLVHVDYVRLRSTNPHTDNGLFHNYESHEDFLDKLYSKDLELFIKFANKITFQIILGKPHKSAALRSEIKNIFEDYYHVKGSSIQIDGTKIILNMSLEIPKQKGVELDENIVVGVDLGIAIPAMCALNTNNYVRLRIGSRDDFLRVRTQIKNQRKRLYSNLKFANGGHGRNKKMQAANRFTDYEKNWVQTYNHMVSKRVVDFALKHKAKYINLECLDGFSQKDLKDNYLLANWSYYQLQQYIKYKAEKYGIVVRFVNPYHTSQNCSCCGHWEEGQRIKQAEFICKNPECKNFGKKINADFNAARNIAMSTYFIDKEEKKKKKTKKAA

SEQ ID NO: 557 - protein, 442 aa, unidentified
  source 1..442: note = Uncultivated organism; MG119-219 effector
MPNLTRKYKLIPVGDKEEVNRVYTCLRKDMENQNKAMNQYMTALYVAQIREASKEDRKELNNIYGRIATSKKGSAYTNDIVFTKGLDILSMAKRKVSEDFGNAMKKGLMYGRISLPTYRQDNPLLIRPDFVRLRSLSLKDYGIYHKYDSPMDLINGLEKDTNPEVYIKFPNGITFKIIFGNPHKGREQRKVFENIFSETYKVCGSSMGIDKNRKIILNLCLEVPKQEHKLDENVIVGVDLGQAIPAMCGLNNNYQIREKIGDKENLLHVRNQMRAKRERLQENLKFSKGGHGRKEKLKALDRLKDRERHFVQTYNHKVSKRVVEFALRHNASQINIEDLSGFGKDKNGNVEELKKKILSEWSYYELQNYITYKAQLHGIVVKKVNPAYTSQTCSCCGQMGIRLSQSEFICQNPDCKCHTLYGKYINADFNAARNIAMSTDYR

SEQ ID NO: 558 - protein, 446 aa, unidentified
  source 1..446: note = Uncultivated organism; MG119-220 effector
MGVSCKKLQIVPVGDDNEVKRVYEYIRNGIYSQYRILNTYMSQVGTLYYKYDRDYKDPGFKDEYNAIFRNTNTAIHDIEQAKGLGMAGNCGMRVKSDFSSALKNGLARGERQLPFYKRDFPLLVPGRFLVFSEKEEDYYDSNGELKTVVNYYIKFVNGIIFKVVFGSKGRSDYRLTSLLSGIVNDVDHYKVCGSSIQISGKKIILNLTTNVEKETVPFIPIKDKKMGIVLGYEQPLVAALSNDDKEYYIGTDIAESIVERRTAIQAYRHRLQVMQTTAKSGHGRKRRMRRVDQQGHYEKNVMRNYNHILSSQVVSFAVKNKVETIFIDDISKDTLIDEPILLRNWSYYQLVEFITYKAKEVGITVITVDFKKNKKEDGEDEADASNKHDFFDRCCVCGEQIDGLAELYDDISVYKDESSFTCPHCRKKVKVRYNKAKNLTYYKNKK

SEQ ID NO: 559 - protein, 452 aa, unidentified
  source 1..452: note = Uncultivated organism; MG119-221 effector
MTETQTITRKIQLIPVGDKEEINRVYTYLRDGIKSQNMAMNQYMSALYSAMVVDVSKDDRKELNHLFSRISTSKLGSAYDETIKFAKGLPSASSVAQKVKQDFDNAMKKGLKYGKLSLPTYRDNNPLLLHVDYVRLRATNPHLDNGLYHNYESHEDFLDNLYSKKLELFIKFANKITFQIVLGNPHKSAEIRSVFKNIFEDYYHIKGSSIEIDGTKIILNMSLEIPKQKVELDEGVVVGVDLGIAIPAMCALNTNDYVKLRIGSKDDFLRVRTQIKNQRRRLYSNMKFANGGHGRTKKMQAADRFADYEKNWVQTYNHMVSKKVVDFALKHKAKYINLECLDGIGQNDLRNNYLLANWSYYQLQQYIKYKAEKYGITVRFINPYHTSQNCSCCGHWEEGQRIKQAEFICKNPECKKFGKKVNADFNAARNIAMSTDFVEKEEKKKKKTKKAV

SEQ ID NO: 560 - protein, 436 aa, unidentified
  source 1..436: note = Uncultivated organism; MG119-222 effector
MITRKIQITPVGDKKEVDRVYAYLRNAIEIQNKAMNEYMSTLYIAEQSELTKDDRKELHNLYGRISTSKKGSAYDKSMEFPTGLPLGVITQAVEADFKKAKKDGVMYGRTSLPTYRADNPLNIHVDYCRLRKLSSHDCGLYHNYKTHLEFLDHLYKKDIEVFLRFANGITFKLNLGNNLQKSYALRVEIQKIFEEEFDVRGSKIQLKKRKTGKGDDILLLLSIETPKKEYKLDENIVVGVDLGVATPAVCALNNKWQPRKYIGDGQTFIHQKQRHKEQRQAIQRNLALCSGGHGRKKKLQKLDKLKQHEGNFTTNYSHKISKEIVDFALKHNAKYINIEDLTGIDTQHNKLLGKWKYFEVQEFTKYKAAQYGIEVRKIVPKYTSQTCSVCGHWEEGQRNGRNFECKNCGTKLNADYNAARNIAMSENFSEEKKKKK

SEQ ID NO: 561 - protein, 465 aa, unidentified
  source 1..465: note = Uncultivated organism; MG119-223 effector
MPNLTRKYELQVVGDKEEINRVYTWLRQGIEAQNKAMNEYMSHLFMAKSCEMSLDDRKELNNLFQRVTNSKKGSGYTKDIEFPVGLPIAASASRKVKQDFDNACKKGLMYGRISLPTYRSNNPLIIHVRFVALRGTKKVDNGIYHNYNSPADLFSALENDAKPEVFIKFCNNITFKFRFGNPYKAKEQREVFKKIFTEEYKVGGSTIEIKGTKIIINLTLEVPQIKMCLDENVTVGVDLGQAIPAMCALNNDYYKRLAIGNVDDFLRIRTQMQEERKRLQCHLRNANGGHGRKKKLRALERLKQREKNFVQTYSHMVSRKVVDFAVKHNAKYINIEDLSGFGRDKYGHVKDDEQAKMVLRNWSYYNLQEKIKYKANIYGIEVRKIDPKFTSQICSVCGEKGIRQTQKEFICINPECSCHKMYQNQKYINADFNAARNIAMSQKFINEDKETAKKNEQKKENSSSN

SEQ ID NO: 562 - protein, 428 aa, unidentified
  source 1..428: note = Uncultivated organism; MG119-224 effector
MSVTTKKIQLLPFTDSKEERERIYNYLRDGIYNQHHILNTYMSQMGVVYYKYNKDIKNPDYKEEVKAIFRNTNMSIKDFTQAKGLAMAANCGMQVKSDFSIAIRNGLAKGERALPFYKRDFPLLVPSRFLNFYSTTESYKDDFGEIKTRDIYAIKFVNGIHFKVVLGSRGRNDFYINSLLDSILNDPDNYHVCGSSIQFSKGGKIILNLTVKISKQAEEYEPVKGRVMGLAMGYDKCLVAAISDKDDAYSIGDYIQESIVEERIKIQEYTQRLQRSMKSAQGGRGRKRKMRSFDKKGSHEKNVVRHFNHILSKTVVEFAKKHKVETIVIEDVNEKELKDHPTLLRNWSFYQLITFISYKAKAYGISVEKSSSKDKPRMQCCFCGEALDKEDIIPKEIEWQNELYFTCPKCGKRIEYSFNKAKNMTVMG

SEQ ID NO: 563 - protein, 453 aa, unidentified
  source 1..453: note = Uncultivated organism; MG119-225 effector
MTSKASQNNTLVVFRKVRLIATGEKSDVNETYKYIRDKMYAQYRVRNLIAGHLYRAYYNAGCNDESPEFNAAADQILSNTSPILDVLDVEYDAKMVEFACQKVSEDFLSAVKNRLEIGTCRFINYKRTQPLQIDTEQVTFHHNYSNAEEFGANKLSEDAEVTVELAQKYKLKAQLGLPYRSGEIRSVFYNLFAGEYKLLSTSIAIDDKAIILTMSIETPYVSRGKKSYQGDADTSVGVILLKYGQAVCAVNNNNYARCYIGDKNHREYVGNFLHRKEKYQARIAECSEKIALYNQMEFIGKTNKIRALKREIEKYEKNIQNLDHAKQRYQSRTYDGIARSVVNFATKNHAKTIRVGLHSEASAPFYIREVIKRLKEKAADYKIELVVHECDCFEQKCSYCGEPLSLLMEDSRLLHCNNPDCFVNKVPARSKTSSGAIEKNFNAARIVAKLSNN

SEQ ID NO: 564 - protein, 428 aa, unidentified
  source 1..428: note = Uncultivated organism; MG119-226 effector
MSVTTKKIQLLPFTDSKEERERIYNYLRDGIYNQHHILNTYMSQMGVVYYKYNKDIKNPDYKEEVKAIFRNTNIAIKDFTQAKGLAMAANCGMQVKSDFSIAIRNGLAKGERSLPFYKRDFPLLVPSRFLNFYSTTESYKDDFGEIKTRDIYAIKFVNGIHFKVVLGSRGRNDFYINSLLDSILNDPDNYHVCGSSIQFSKGGKIILNLTVKISKQAEEYEPVKGRVMGLAMGYDKCLVAAISDKDDAYSIGDYIQESIVEERIKIQEYTQRLQRSMKSAQGGRGRKRKMRSFDKKGSHEKNVVRHFNHILSKNVVEFAKKHKVETIVIEDVNEKELKDHPTLLRNWSFYQLITFISYKAKAYGISVEKSSSKDKPRMQCCFCGEVLDKEDIIPKEIEWQNELYFTCPKCGKRIEYSFNKAKNMTVMG

SEQ ID NO: 565 - protein, 473 aa, unidentified
  source 1..473: note = Uncultivated organism; MG119-227 effector
MSVSCKKLQLIPLGDKEERNRVYTYLKNGIYSQYRILNTYMSQVGTLYYSCNRNVESEEFTSGFDQIFKDTEAAYRNLGFMAFGDYLRKMEEDYKKHMKDLKDKEYKESYNAIFRNTNEAIWDIEQAKGLGMAGNAGMRVKQDFSTALKNGLARGERQLPFYKRDFPLLVPSRFLTFYSEDEVYTNEQTGEECERTNYCIKFVNGIRFKVVIGSQGKRDLYLLPLLDSILHDPENYKVCGSSIQITKQGKIILNLTTNINKREKEYEPVKGRVMGLSMGYDKCLVAALSDKDKMYEIGTYDKDGYIERRIRLQNERQLLQMKKKGPEGSGHGRKRKMRDFDNMRKRESNIVRYYNHRLSKQVVEFAKKNKVEAIVIEEINASDLNEHPTMLRNWSYYQLQDFIKYKAGKGIKVMVSSKEENRKVCCNCGKPFEKKDVLPKEIEWTGTIHFTCPNCKKQIEYSFNKAKNMALNG

SEQ ID NO: 566 - protein, 446 aa, unidentified
  source 1..446: note = Uncultivated organism; MG119-228 effector
MGVSCKKLQIVPVGDDNEVKRVYEYIRNGIYSQYRILNTYMSQVGTLYYKYDRDYKDPGFKDEYNAIFRNTNTAIHDIEQAKGLGMAGNCGMRVKSDFSTALKNGLARGERQLPFYKRDFPLLVPSRFLVFSEKEEDYYDDNGELKTVVNYYIKFVNGIVFKVVFGSKGRRDYRLTSLISGIVNDVDHYKVCGSSIQISGKKIILNLTTKVEKETVPFIPIKDKKMGIVLGYDQPLVAALSNDDKEYYIGTDIAESIVERRTAIQAYRHRLQVMQTTAKSGHGRKRRMRRVDQQGHYERNVMRNYNHILSSRVVSFAVKNKVETIFIDDISKDTLIDAPILLRNWSYYQLVEFITYKAKEVGITVITVDFKKNKKDDGEDEADASNKHDFFDRCCVCGEQIDGLAELYDDISVYKDESSFTCPHCRKKVKVRYNKAKNLTYYKNKK

SEQ ID NO: 567 - protein, 452 aa, unidentified
  source 1..452: note = Uncultivated organism; MG119-229 effector
MTETQIITRKIQLIPVGDKEEINRVYTYLQDGIKSQNMAMNQYMSALYSSMVVEISKDDRKELNKLFGRISTSNLGSAYDKTIEFAKGLPMGGAIQQKVKRDFDNAMKKGLKYGKLSLPTYRENNPLLVHVDYVRLRTTNPHSDNGLYHNYESHEDFLNSLYSKDLELFIKFANKITFQIVLGNPHKSAEIRSVFKNIFEDYYHIKGSSIQIDGKKIILNMSIEIPKQKVELDENVVVGVDLGLAIPAMCALNTNDYVRLRIGSRDDFLRVRTQIKSQRRRLYSNLKFAHGGHGRSKKMQATDRFTNYEKNWVQTYNHMVSKRVVDFALKHKAKYINLECLDGFSKKQLRDNYLLANWSYYQLQQYIKYKAEKYGIVVRFVNPYHTSQNCSCCGHWEEGQRVKQAEFICKNPECKNYGKKINADFNAARNIAMSTDFVEKEEKKKKKTKKAA

SEQ ID NO: 568 - protein, 465 aa, unidentified
  source 1..465: note = Uncultivated organism; MG119-230 effector
MPNLVRKYELQVVGDKEEINRVYTWLRKGIEAQNKAMNEYMSHLFMAKSCEMSLEDRKELNNLFQRVTNSKKGSGYTKDIEFPIGLPIAASASRKVKQDFDNACKKGLMYGRMSLPSYRNTNPLIVHVNFVALRGTKKPDNGIYHNYENPAKLFEALENDMKPEVFIKFCNNITFKFRFGNPYRGKEQREVFKRIFTEEYKVGGSTIEIKGTKIIINLTLEVPKVEAVVNEDVVLGVDLGQAVPAMCALNNDYYKRLAIGSADEFLRIRTQIQNERRRLQHALKDSKGGHGRQKKLRALDRLKEREKNFVQTYSHMVSRRVVDFAIKNGAKYINIEDLSGFGRDKYGHVKDDEHSKMILRNWSYYDLQEKIKYKAAIHGIEVRKIDPKYTSQTCSVCGEKGLRQTQKEFICTNPECVCHKIYSNQKYINADFNAARNIAMSQNFIEEDEGSKKKSEQKKENSNGN

SEQ ID NO: 569 - protein, 429 aa, unidentified
  source 1..429: note = Uncultivated organism; MG119-231 effector
MVITRKIALTVVGNKEEKDRVYTYIRDGIKNQNLAMNQYMSALYVANMQDISKDDRKELNHLYTRISTSKKGSAYSTDIQFPKGLPCTSSLGQEVRAKFKKACKDGLMYGRVSLPTYRANNPLLIHVDYVRLRSTNPHNDTGLYHNYESHTEFLEHLYKNDCEVFIKFANNITFQLFFGQPHKSHELRSVIQKVFEEYYSVCGSSIEISKKGKIMLNMCIEIPVEKKELDENIVVGVDLGISTPAMCGLNCNDYVREGIGSKDTLLSKRTQLQRQYRELQGRMKMTNGGHGRGKKLKKMDDYRNHERHFVQTYNHQVSKKIVDFALKYKAKYINVEDLSGFGNRDTNQWVLRNWSYYELQQYITYKAQKYGIEVRKVKPYLTSQTCSHCGHYEPGQRLDQAHFECKNCGLKINADFNASRNIAMSTEFV

SEQ ID NO: 570 - protein, 451 aa, unidentified
  source 1..451: note = Uncultivated organism; MG119-232 effector
MSNTQILTRKFQIFPVGDKEEINRVYQYIRDCQYVQNKAYNILISNVYSAIISGKSKEEINLIYKKGGRKPKEDNPDYSLYNYNDFKVPVGLTIIGALPQTVKNDLKTAQKAGLFKGNSALPNRRRTASLNIQPVYVRLRDINLRNPKITFKTGIYHTYSSYNDFLDNLYSKRELDIYLAFANNITFKFVLGNPYKSYQDRNILQHIFEEDYLVKGSSIFFDKTNKKIMLNLSMEIPKKEIELKEDVVVGVDVGLAIPAVCGLNINSYAREYIGSKDDFLRVRTQISNERKRVQKNLKMVKGGHGRKKKMKSLDRFTLKEKNFVQTYNHMVSKRVVDFAVKNHAKYINLENLKGYDANIFILRNWSYYQLQQYIKYKAEKYGIETRMIDPYHTSQICSCCGHWEEGQRINQSTFICKNPECKNFGVKINADFNASRNIALSTNFISADSAE

SEQ ID NO: 571 - protein, 477 aa, unidentified
  source 1..477: note = Uncultivated organism; MG119-233 effector
MAVLTRTLEMMPVGDKAERDRIYTWIRSFMDAQSRMMNQYMSALYIAAIQEISKEDRQELNKLYNRIPTSSKGSAFDKEIELPKGLGAAYGQTVRADFDTSCENGLLHGRVALPTYKKDYPIIIPPLYVQLRKVNQETKGKNCGFYEEYGSYNDLYDSLTQGEPEIFWSFVDKMIFKIRFGNPYKSAFLRSEILHFLEGEYKAMGSSLSINNKGKIILSLTMEIPEKKVKLDENTVCGVSLGFMHPIVCSVNNNFYKKEFIGDYDVFVDTRVRLQDQRRELQKKLKYASSSHGRVKKLRKLAILRKREANFAKNMNHKMSSDIVKFAVKNEAKYINMQDLTSFGKDSRDNIKSGYEFALRNWSYFQLQQMVTYKAASYGIIVRKISNDNLSNTCSICGAEGIIEDGAFICSDPNCLSHDKYEKARKNGKGNYYFNSDFNISRNVSISENFTTKKRKSKAEKKEERKAKKEKEKLDKS

SEQ ID NO: 572 - protein, 468 aa, unidentified
  source 1..468: note = Uncultivated organism; MG119-234 effector
MEEKKTHVISRKVRLYVVPHEGTPEEQEAEINRVYTELRSGMEAQNKAYNILVSAIYSATMSGASMDEIKMICKRGSRNPKEDDPDYSLYDFNEIKFMKGLPSASKVNRYAEADMKKAWKDGLKYGVVSLQTKKMDAPLWVIPENLKYRHNCPSYTLFLELLMNGKTDIFIDFVQGITFGVVLGNPYKGHRNRLMWKRIFEGEYKIGTSSIQIAKEGKIILNLALTVPVEEHKELNKDIVVGVDLGIKIPAVCALNNNEYEREMIGSINDFLRVRTSMQAERKRIKKALKTGASGGHGRKKKLKNLDNIERREHNFATTYNHMVSKRVVDFALKNNAGVIHIENLEGFGKNEKTQKVLRNWSYYQLQQDIIYKAEANGIEVKKINPYHTSQICSICGSEEPGQRTSQTTFICANPKCRSHKIYFNDRHGKEEFNADFNAARNIAKSTEYQTGKKSKRQKKEQHENING

SEQ ID NO: 573 - protein, 447 aa, unidentified
  source 1..447: note = Uncultivated organism; MG119-235 effector
MSTTTKKIQLIPVGDKEEKDRVYDYLRDGIYNQHKILNTYMSQVGTIYYKYNKDFNNPDFKEELKAIFRNKNEAIYEFTQAKGLGMAGSCGRRVNQDFSTALRNGLARGERALPFYKRDFPLLIQGRFLTFYTGEDSYQDEEGNEKTVNAYFIKFVNGIHFKCVLGSRGRKRPDLYLPRLLQSIVEDPENYRICGSTIQFSKDNKIILNLTVDINKTVEDYKPVKGRVMGLTMGYDKCLVAALSDSDEMYTIGDYIKDSVIEKRIKIQKDTHRLQMSMREDAKGGHGRKRKLEKLEQHKKYEKNVIQDFNHRLSKSVIDFAKKHNVEIIVLEGISKEELEKYPTLLRNWSYYQLQTYIKYKAKAEGIDVRVIEDKQADTDDKTQKGRKKKKALDEIVRKTCCACGCSLDRDDIIPKQIEWCHELSFTCPECGKTIDYSYNKAKNMIS

SEQ ID NO: 574 - protein, 403 aa, unidentified
  source 1..403: note = Uncultivated organism; MG119-236 effector
MNEIKMICKRGSRNPKEDDPDYSLYDFNEIKFMKGLPSASKVNRYAEADMKKAWKDGLKYGVVSLQTKKKDAPLWVIPENLKYRHNCPSYTLFLELLMNGKTDIFIDFVQGITFGVVLGNPYKGHRNRLMWKRIFEGEYKIGTSSIQIAKEGKIILNLALTVPVEEHKELNKDIVVGVDLGIKIPAVCALNNNEYEREMIGSINDFLRVRTSMQAERKRIQKALKTGASGGHGRKKKLKNLDNIERREHNFATTYNHMVSKRVVDFALKNNAGVIHIENLEGFGKNEKTQKVLRNWSYYQLQQDIIYKAEANGIEVKKINPYHTSQICSICGSEEPGQRTSQATFICANPKCRSHKIYFNDRHGKEEFNADFNAARNIAKSTEYQTGKKSKRQKKEQHENING

SEQ ID NO: 575 - protein, 448 aa, unidentified
  source 1..448: note = Uncultivated organism; MG119-237 effector
MITRKIQLYPIGDEKEVDRVYTYLRDSIKSQNKAMNQYMSNLYVEELLNASKEDRKELNALYTRIPTSKKGSAYSLDLEFQKGQMLGYITRAIDSDFKKSLKDGLMYGKVSLPVYSNSNPLLVHVNYCNLRSKVQKQTGTNTGIYHNYESHQEFLSHLYKTDIEIYLQFCNNILFKLNLGDSLRKSREIRSVIQKIFEEEYIIHGSQIQLKKRKTGKGEDIFLLLSIETPVVKHELDENIVVGVDLGVANPAVCAVNINGNYKFLGDGETFLHYKQKSKAERRRLQRALKYTNGGHGRTKKLKALDRLKERESNFTTTYSHKLSKDVVDFAIEQHAKYINLEDLTGIDDKDGKKLLDCWKYYELQQFIIYKATHHGIVVRFINPKYTSQTCSKCGNHAEGQRDGHSFTCKDCGEELDADLNAARNIAMSTNFVERGKKKKKKNKTDNK

SEQ ID NO: 576 - protein, 439 aa, unidentified
  source 1..439: note = Uncultivated organism; MG119-238 effector
MGTTTKKLQLYPIGEKEEVDRVFTYLRDGIYNQHCILNTYMSQVGCLYYKYNKDFKNVGFKEEFNAIFRNTNEAIKDFTQAKGLGMAGNCGMRVKQDFSTALKNGLARGERNLPFYKRDFPLLVPSRFLTFYTIDSVDKKTGEERTVYCIKFVNGINFKIVIGSQGKKDFYLLPLLDSIINDPDNYHVCGSSIQITKKGKIILNLCVKVSKKAEEYHPKQGRVMGLALGYDKCLVAALSDSDKMFTIGDDIKDSVVEKRIKIQAYNQALQKALKDAKGGHGRARKLAKFNERDRYEKNVVRHFNHVLSKKVVEFAKKQKAETIVIENIDKADLESYPVLLRNWSYYQLCEYIKYKSGDDINVILAGEGKKSKKKAEGDTDTATSAELRKICCKCGCKLDESVILPEKIEWTNELYFTCPECKEKIEFSYNKAKVMSMKG

SEQ ID NO: 577 - protein, 467 aa, unidentified
  source 1..467: note = Uncultivated organism; MG119-239 effector
METILTRKIMLKVVGDNAEKNRVYSYLRNGQYINALMKNQYISALYAAYMTGASKEEIKELNTLYSRVPGSGKGSAYSFDTKLYPTGLPLASSVQHLGKDLFSKACKDGLLHGKVSLPTYKADGPMPIPKYLVSRRGTVHRTSGNKEGFMDAGFYHEYASDAEFFSELQSKRVPDVYIKFTNNIVFKVRFGNKNRTDQERALISDIFAGRFPVCDSSIGVEDKDIILNLSMKIPECKNELDKDTIVGVKLGIATPAVCALNNDYKKREYIGTYEEFTGHRNKMAARKRILTRDLAQCKGGHGRSKKLAKLEKNSAREADYAKTYNHRISRTIIDFALKNKASVIQIEKLSSLNKKKKKYFDDTKEKKEERNLLGNWSYYQLQQQIEYKAKKEGIKIRYVNPAYTMQTCSCCGMLGERVGNDFRCISSGCKFNGKIRNADFNAARNISMSTDYIDNEKETAEESEETA

SEQ ID NO: 578 - protein, 442 aa, unidentified
  source 1..442: note = Uncultivated organism; MG119-240 effector
MKYINVVRKIQAVPCGDRNEANRVYQYLRSGTEAQNKAMNQMMNGLVMAHVSEMQKDDKKEFVKYFNRTGTTKLESAYVDVEFATGIPISAFVQKVKADFDTALKNGAAYGRVRFPEYKSDNPLLVHIDWVRLRKTNPHNDFGIYHCYPTDEEFEKHLYSKDLEIFWKFANGITFKLVFGNLKRSCELRSVFKNIFNETYEIRGSSIQINSKNQIILNMTIKMPVYEASLDENTVVGVDIGMAIPAMCALNNNPVCRQRIGSFDDFMRVKTQIKKERSRIQSSIRFGSGGHGREKKLKSMQKFRSRESNWAMTYNHTVSRKVVDFALKNHAKYINLEDLSGIASQEKSNKLLRDWTYFQLQQFITYKAEKVGIVVRKIDPYHTSQICSCCGHWEEGQRISQDKFICKNPECKNFGKIVNADFNAARNIAMSTAFVDEKKLSA

SEQ ID NO: 579 - protein, 465 aa, unidentified
  source 1..465: note = Uncultivated organism; MG119-241 effector
MPNLTRKYELQVVGDKEEINRVYTWLRQGIEAQNKAMNEYISHLFMVKSCEMSLDDRKELNNLFQRVTNSKKGSGYTKDIEFPVGLPIAASASRKVKQDFDNACKKGLMYGCISLPTYRSNNPLIVHVRFVALRGTKKVDNGIYHNYNSPADLFSALENDTKPEVFIKFCNNITFKFRFGNPYKAKEQREVFKKIFTEEYKVGGSTIEIKGTKIIINLTLEVPQIKKCLDDNITVGVDLGQAIPAMCALNNDYYKRLAIGNVDDFLRIRTQMQEERKRLQCYLRNANGGHGRKKKLRALERLKQREKNFVQTYSHMVSRKVVDFAVKHNAKYINIEDLSGFGRDKYGHVKDDEQAKMVLRNWSYYDLQEKIKYKANIYGIEVRKIDPKFTSQICSVCGEKGIRQTQKEFICINPECSCHKMYQNQKYINADFNAARNIAMSQKFINGDKETTKKNEQKKENSSSN

SEQ ID NO: 580 - protein, 450 aa, unidentified
  source 1..450: note = Uncultivated organism; MG119-242 effector
MGQNITRKIQLVPIGDKKEISRVYNYLSKHIEAQNKAMNQYISSLYAAHLLDISEEDRKELRNLYSRIPTSKKGSGYDTSIEFQKGIPLSNITRSIESDFKKSLKDGLMYGKVSLPTYKKNNPMLVHVNYCDLRDKVKERTGKNTGIFHNYKNHEEFISHLYKTDLEVFLQFTNNILFKFHFGESVKKSSEIRSVMRKIFEREYTIHGSQIQLKERKTGDGDDIFLLLCIEVPEVTHELSEDTVVGVDLGVANPAVCALNTKRYPYKYIGDGSIFIHHKQKYKNERTRLQRNLKYTSGGHGRKKKLRALNRLRDSESNFATTFSHKISKDIVDFAISNNAKYINIEDLKGIDTKNTKLLECWKYFELQQFITYKAARHGITVRKINPEYTSQTCSICGNREDGQRDGRNFHCKACGAELDADYNAARVIAMSTNFVEDKKKKKKKKEESE

SEQ ID NO: 581 - protein, 469 aa, unidentified
  source 1..469: note = Uncultivated organism; MG119-243 effector
MPTLTRKVKLHVVGDKEEVSRVYDYIRLAMNATYKCFNECMTALYIAQVKEDTTDDRNELNCLYSRQTYTKKETAFTNDIVFPTGLALASYINHEANLKFKTSIKNGLMHGCVSLPTYKKDCAVPLRCEFVSFAENKDKGLGFYHEYADVNELINALEYDNNPKVFLKFPNDIRFEVVFGNPYRGREQRSVFSKIFLGEYKTRGSSIQINSKSRIILNLTMEVPKKEMEQIEGRVVGVDVGLAIPAMCAINDDNYARSAIGDINDFLRVRTQIKSQRRRLQKSLKNTSSGHGRIKKLKPLEKIAEKERNFVNTYNHMVSKRVVDFAVKNGASQINIEDLSGFAKDKNGKSVEDANMKRILSNWSYFELQQQIRYKAGQYDIKVRTVNPAYTSQTCSYCGQIGKRETQSKFVCTNPDCGCHKIYKDGWFNADFNAARNIALSTDYTDDEDGKKSKKKPAKAKVKKWNEKD

SEQ ID NO: 582 - protein, 444 aa, unidentified
  source 1..444: note = Uncultivated organism; MG119-244 effector
MNNIVTRKIGLFVVGDKTESDRVYKYLRDGMASQNRAMNQYITALYFADQNEATKDDRKELNRLFSRISSSKKGSAYDTSIEFAKGLPSGSSVTQKVEADFKNAMKKGLKHGIVSLPTYKKDNPLIIHRDYVRVRFTNPHHDNGLFHEYESHEDFLKHLYSNDLDLFIKFANGITFKLYLGKPKKSAELRSVLKNIFEDYYQIQGSTIEITNGKIILNLSVSIPQKEIELDENTVVGVDVGMACPAVCAVNKNDVYKQFIGSADDFIRVRTQIQNEYRRLKQSVAFNSSGHGRSKKAKPTDRFHEYESNWVKTYNHKVSKKVVDFALKNKAKYINIENLSGIAQDEKNKAFVLRNWSYYQLQQFITYKAAKYGIIVRKVDPAYTSQRCSCCGHLEKGQRVSQSDFICKNPKCKNFGKIVNADFNAARNISMSTKFVKDDVEVGT

SEQ ID NO: 583 - protein, 451 aa, unidentified
  source 1..451: note = Uncultivated organism; MG119-245 effector
MSNTQILTRKFQIFPVGDKEEINRVYQYIRDCQYVQNKAYNILISNVYSAIISGKSKEEINLIYKKGGRKPKEDNPDYSLYNYNDFKVPVGLTIIGALPQTVKNDLKTAQKAGLFKGNSALPNKRRTASLNIQPVYVRLRDINLRNPKITFKTGIYHTYSSYNDFLDNLYSKRELDIYLAFANNITFKFVLGNPYKSYQDRNILQHIFEEDYLIKGSSISFDKTNKKIMLNLSMEIPKKEIELKEDVVVGVDVGLAIPAVCGLNINSYAREYIGSKDDFLRVRTQISNERKRVQKNLKMIKGGHGRKKKMKSLDRFTLKEKNFVQTYNHMVSKRVVDFAVKNHAKYINLENLKGYDANIFILRNWSYYQLQQYIKYKAEKYGIETRMIDPYHTSQICSCCGHWEEGQRINQSTFICKNPECKNFGVKINADFNASRNIALSTNFISADSAE

SEQ ID NO: 584 - protein, 458 aa, unidentified
  source 1..458: note = Uncultivated organism; MG119-246 effector
MSKEKTLIMVRKVKLHPVGDKEEVSRVYKYIRDCSYVTKIVKNKSMSFLYSAFVTGMDNEKRKEEYRKYTRKPNPNKEGYSAYEGIELPDIKGLPVFGDASHAAQDEFKNDLKNGLASGKISLRNTKQGVPVNINKCYLQKRDDPDYPGQKHGGFYHEYPDDREFVEHLDKDKVELYFQFPNNIYFRVDLGNPYNGKEIRSCFAKFWSGEYKTVGSKIMISKRNEIILSLAFEVPVENNDLNSDIVLGVDVGMAYPAYAALNNDNYARRSFGSFDELAKRKESFNAQRKRMQHGLSFAKGGHGRKRKLVKLDDLKTKEKNYTDTYIQQISSEIIRYALSKRAGVIQMEDLSFVREKSDDSVFVMRNWCYADLQSKIEYKAAKSGIEVRYINPAYTSKTCSVCGKAGQRLSQKEFICSDPDCKSHKMYRDNGKYFGADFNAARNIALSTDFSDKKKSRV

SEQ ID NO: 585 - protein, 470 aa, unidentified
  source 1..470: note = Uncultivated organism; MG119-247 effector
MATLTRKIQLYVVGDKKEVSRVYDYIRGATDATYKAFNECMTALYVAEKSDETKEGKKWLNEYYSRQTYTKYKSGFSDNIVFPEGLALASYINNLAKDKFSIAVRNGLMYGCVSLPTFKKDCAVPLHVKYVAVKGTKSDSHGLYHEYADVNDLINELENGDKPKVFLKFPSKITFGIVFGNPYDGREQRSVFSKIFLGEYQVCGSSIQINSKNKIILNLTLKVPDKKIEQVEGRVVGVDLGIAIPAMCAMNDDDYSRSSIGKINDFLRVRTQIRNQRMRLQKGLKNTSSGHGRTKKLEPLERFKEKERNFVQTYNHMVSKRVVDYAIKNGAAQINIEDLSGFGKDKNGKSKEDEKTKKILSNWSYFESQQYIRYKAEMHGIKVRTVNPAYTSQTCSYCGQRGKREEQSKFVCVNPDCKCHKMYKKGYLDADFNAARNIALSTDYTDDENGKKKKKKSSKKKDVNKSGKEE

SEQ ID NO: 586 - protein, 451 aa, unidentified
  source 1..451: note = Uncultivated organism; MG119-248 effector
MADTQILTRKFQIFPVGDNDEINRAYQYIRDCQYVQNKAYNILISNVYSAIISGKSREEIDLIYKKGGRKPKEDNPDYSLYNYNDFKVPVGLPIIGGLPQTVKSDLKIAQKAGLFKGNSALPNKRRTASLNVQPIYIRLRDINLRNPKINSKTGIYHTYSSYNNFLDNLYSKRELDVYLAFANNITFKFVLGNPHKSYQDRNILQHIFEEDYLVKGSSICFDKTNKKIMLNLSMEIPKTKIELKEDVVVGVDIGLAIPAVCGLNTNSYARESIGSKDDFLRVRTQISNEYKRVQKGIKMNKGGHGRKKKMKALDRFTLKEKNFVQTYNHMVSKRVVDFAIKNHAKYINLENLEGYDASKFILRNWSFYQLQQYIKYKAEKYGIETRMINPYHTSQICSCCGHWEEGQRIDQATFICKNPKCKNFGIKINADFNASRNIALSTNFISADSVE

SEQ ID NO: 587 - protein, 525 aa, unidentified
  source 1..525: note = Uncultivated organism; MG119-249 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTPEQKISRTEFVCADPDCKSHTNYKKGFTADFNHARNVAMSTLFMENGSFSKKSMDEAAEYYGISEKYKA

SEQ ID NO: 588 - protein, 579 aa, unidentified
  source 1..579: note = Uncultivated organism; MG119-250 effector
MKKNNKESNTYIKAMKIRIVPICDIEEENLRDKYYKELYQYLRDSIWAQNAALNYGLAMTRDAYVLHKNEDKIKDIYFKLAHQTPNPSMASEARLKEVYEAAPITQEYIDNKVKEFKKSNNKKKKPLSDEGVKKKTDSINNMYKKFIGLSKEDIQGLIDKLDDYCAYPEEIYEKFANGFSTPAYITQQIKDYWNTEGVMTKVIYSMSDKLRSVKNSNPLTIPPNLFYNKKNDLIGITHSYNTYLEFVEALMNVYDANLFLELPYKKGYDKMKFKLILGNPCKSHEVRISLQRIFEEYYKIRGSKIGFVRNKKTGKNTDLVLYLSVEIPKDTSIELDENTVVGVDLGLAVPAVCALNNNPYPRKYIGKKLELLKKRTQFKNRRKKLQSELRDARGGHGRRRKMKAMDRLSELEKNFADTYCHKVAKKVVMFALKNKAKYINLEMLKGYRADEKVLQNWSYYRIQMYIKILAERYGIIVRFINPCYTSQVCSVCGNWHPGNRPKGDKGQKYFDCHNDKCESHTKNKGEGYTLNADYNAARNIAMCDLFMDNKSEITEEDKRIAREKYGIPEPIKKKEKLAA

SEQ ID NO: 589 - protein, 525 aa, unidentified
  source 1..525: note = Uncultivated organism; MG119-251 effector
MAEKMTMITKKCVLIPERSDSLLWERRVYDFTVKDYEQRISFYESQLESKNKEVKRNAKIRLEILNKELDAFLDNPEITQKVINNYTYGLVRTAMSEEAARKNYIIEYVKSILIAEHAYAMDFRDRNKRISQILQYAYRKKGSSKGSLFDETEIGNILGSYGIGFSQMLTRKIQDCCNKGALEGRASFPFYKEDSPFTVTKAAMGFSHEYDSYEELCEHVKDCKLYFDYGGNGKPHIARFQIDLGHGRNRDKLMATLLKVYSGEYAYCGSSIQISKNKIVLNLSLQIPKEEKDELDKDTVVGVALGLVQPAVCALNNDQYVRLPIGDERDLLDNKLQIQERRRRLQSALRETSGGHGRKKKMQALNRIAKTEKNFTETYCHMVSKRIVEFALQNNAKYINMENLSGYNTDKFILRNSYASKIIMYTTYKAGHYGIEVRLVNPCFNAQVCSICGNWTSEQKISRTEFVCADPDCKSHTNYKKGFTADFNHARNVAMSTLFMENGSFSKKSMDEAAEYYGISEKYKA

SEQ ID NO: 590 - protein, 426 aa, unidentified
  source 1..426: note = Uncultivated organism; MG119-252 effector
MILTRKIQLYPVGDKEEVNRVYQYIRDGIFNQNKAMNQYMTALYVSTIQDISKEDRQELNRLYTRIATSKKGSAYDLDVEFAKGLPTTASLGQKVRQDFSKACKDGLLYGKVSLPTYRKDNPLLVHVDYVRLRETNPHMDNGIYHNYESYDEFISKLYDKDLEIFIRFANGITFKMILGQPHKSIYLREEIKKIFDGEYNVCGSSIQFNDKKIILNMSMDIPKISRELDENTVVGVDLGIAVPAVCGLNNSNVKKQFIGSKEDFLRIRTQLQAQRKRLQKNVKITNGGHGRNKKLKALDKLSNKERNFVKTYNHYVSKQVIDFALKNNAKYINMEDLSGFNTNKFILRNWSFYELQQFITYKAQRYGIEVRKVNPYHTSQICSKCGHWEEGQRDTQDHFKCKACGYEDNADFNASKNIAKSIDFRK

SEQ ID NO: 591 - protein, 414 aa, unidentified
  source 1..414: note = Uncultivated organism; MG119-253 effector
MANVLTRKIQLVPVGDKEEINRVYNYIREGQYSQYLGLNRLMSELVVTFYKNDRKLDEKFNEERKNILSNSNTILNDILFPKGCDTKSHIVQKVKSDFSKALKNGLARGEVAVTNYKRTSPLLVRGRDLKFSYDYATLNEFEQNLLNTDLKVYINFVDKIKFKIVFGNPHKSQALRLEIREILLDNYKVKGSSLQIDGKKIILNLSIAVPTKKIELDKKVTVGVDLGLAIPAVCALNTNDYIRKSIGSADDFLRQRTKIQKQKKAIQKNLVQVKGGHGRKKKLNHLEKVRKNEEHWVRTYNHFVSKQVVDFAIKNKAKYINVEDLTGFDNSKFILRNWSYYQLQEFITYKANNVGIQVRKINPYHTSQTCSCCGHWEEGQRISQSKFICKNYGKEINADFNAARNIAQCSDFIK

SEQ ID NO: 592 - protein, 462 aa, unidentified
  source 1..462: note = Uncultivated organism; MG119-254 effector
MGNDRMTICRKIKLFPVGDKEEINRVYDFIRNGQYAQYQACNLLMGQLMSEYYKYNRDIKNEEFKARQKEIMTNSNIILKDIDFATGVDTPSAVTQKVKQDFSTALKNGLARGERTVTNYKRTNPLITRGRNLTFYHEYETYHDFLDKINDSDLAVYVKWVNKIVFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNISGGHGRAKKLKALERLQKAETHFVETYCHIISKRVVDFALKHNAKYINIENLTGYDTSDFILRNWSYYKLQDYITYKAAKYGIEVRKINPCYTSQICSVCGNWEFGQRKSQSVFECANENCDSHKKYEKIGFNADFNAARNIAMSTLWMESGQVTEKSKQEAREYYDISKKYEQSKSDSENNKVA

SEQ ID NO: 593 - protein, 441 aa, unidentified
  source 1..441: note = Uncultivated organism; MG119-255 effector
MVLTRKIQLIPVGNKEEINRVYKYLQDGIYSQNKAMNQYISALYVSAIQDISKEDRSELNKLYGRISTSKKGSAYDKNIIFPKGLPIGALTRKVRQDFSKSCKDGLLYGKVSLPMYKKDNPLLIHVDYVRLRKTNPHQDSGIYYEYDSYSDFLEHLHKNDLEVFIKFANQITFKMIFGSPHKSYTLREEIKQIFEENYKVCSSTIQIDGKKIILNLSMEVPKNDVELDENIVVGVDLGIAIPAMCALNNNEYIRKSIGSKDDFLRVRTQLQSQRRRLQKTLKISNGGHGRNKKLQALNKLKNRERNFVRTYNHYVSKQVIDFAVKNKAKYINVEDLSGFDSSKFILRNWSFYELQQFITYKAAKYGIEVRKVNPYHTSQICCKCGHWEEGQRIDQAHFVCKKCGNETNADFNAARNIAMSVDFIDKKKEVETTCNNMQLTA

SEQ ID NO: 594 - protein, 418 aa, unidentified
  source 1..418: note = Uncultivated organism; MG119-256 effector
MGDTQTITRKIQLFPVGDDEEISRVYKYIRDGMYAQYQASNLLMGQLASEYFKCNRDIKSDSFKNAQKEIFKADNPLLKEIEFPKGNDTLSAVTQKVKQDFSTALKNGLARGERTITNYKRTNPLLTRNRSLKFCYEYESYHEFLDKLFTNELEVFVKWVNKIKFKVVFGNPHKSHELRCVIQNIFEEVYKVQGSSIQFDNTGKKIILNLSLAIPRKRMELDENTVVGVDLGVAVPAVCALNNDEFAREFIGSKDDFLRVRTRIQAQKRRLQKSLKFSSGGHGRNKKLKPLKQFEDYEKNFVQTYSHMVSKHVVDFAVKHNAKYINIEDLEGYRSNDFILRNWSYYKLQEYITYKAGIYGIEVRKVNPYHTSQICSCCGHWEEDQRIDQLHFKCKLCGAELNADFNAAKNIAKSTDFR

SEQ ID NO: 595 - protein, 401 aa, unidentified
  source 1..401: note = Uncultivated organism; MG119-257 effector
MYLVRKIRLIVIGENKEDVNEKYKFIRDAQYAQYRGLNYMMGQVGALYYSCDRNISSDEFKTQYMEIFRASNQVVDGIEWGTGIDTRSLVGMRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 596 - protein, 426 aa, unidentified
  source 1..426: note = Uncultivated organism; MG119-258 effector
MILTRKIQLIPCTEGMTTDEAKVEVNRIYQLLRDGIYAQNKAYNIFISRMYTATLLGASKEELKEIRLKGERTPKESDSNYSLYDFDKIKFIKGLPQASALGEQALKALKKQQKDGLYKGKVSLACRKLDAPMWIKTKFSFFHKYNDYQEFLDHLYCDDLKIYMKFVQGIVFETVLGNPHKSETIRAEFQQIFEGHYKLCSSSLQIKDKKIMLNLAIDIPEKEIELNDETVVGVDIGIAIPAVCALNSREYIHKSIGSAEELLRIRTQLQSQKRRLQKNLKNTTGGHGRSHKLAPLDKLAKRERNFVQTYNHMISKTVVEFAVKNKAKYINLEDLSNYKDNGSEFILRNWSYFELQSQIEYKAKMHGIIVRKINPYHTSQICSKCGHWEEGQRISQSKFKCKSCGYESNADFNAARNIALSTDFIE

SEQ ID NO: 597 - protein, 457 aa, unidentified
  source 1..457: note = Uncultivated organism; MG119-259 effector
MTICRKIKLFPVGDKDEINRVYDFIRNGQYAQYQACNLLMGQLMSEYYKYNRDIKNEEFKARQKEIMTNSNIILKDIDFATGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNTSGGHGRAKKLKALERLQKAEAHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSHKKYEKIGFNADFNAARNIAMSTLWMESGQVTEKSKQEAREYYGISKKYEQNKNDSENNKVA

SEQ ID NO: 598 - protein, 457 aa, unidentified
  source 1..457: note = Uncultivated organism; MG119-260 effector
MTICRKIKLFPVGDKEEINRVYDFIRNGQYAQYQACNLLMGQLMSEYYKYNRDIKNKEFKARQKEIMTNSNIILKDIDFATGVDTPSAVTQKVKQDFSTALKNGLAKGERTVTNYKRTNPLITRGRNLTFYHEYETYQNFLDKINDSDLAVYIKWVNKILFKVVFGNPHRSLELRSVVQNILEENYKVQGSSIEIDGKSIILNLSISIPKQLRELDENTVVGVDLGIAVPAMCALNNNLYERLAIGNAEDFLRIRTKMQAQRRRLQKSLRNTSGGHGRAKKLKALERLQKAEAHFVETYCHMISKRVVDFALKHNAKYINIENLTGYDTSDFILRNWSYYKLQDYITYKAAKYGIEVRKINPCYTSQICSVCGNWEFGQRKSQSVFECANENCDSYKKYEKIGFNADFNAARNIAMSTLWMETGQVTEKSKQEAREYYGISKKYEQNKNDSENNKVA

SEQ ID NO: 599 - protein, 401 aa, unidentified
  source 1..401: note = Uncultivated organism; MG119-261 effector
MYLVRKIRLIVIGENKEDINEKYKFIRDAQYAQYRGLNYMMGQVGALYYSCDRNISSDEFKTQYMEIFRASNQVVDGIEWGTGIDTRSLIGMRIKQDFSTALKRGLAKGEISLPNYKRSYPLLTKGRDIRFYTDYTEGELVHDMVLEPDFNLYIKWVHKIHFKVVLGNPYRSRFLREELWKVLCGEYKIAGSSLGFDKNGKLILNLSMDIPVNKEKKENMNNVLGVRLAYAKPVVAVVNSTGRCFMIGDGERLIGYRKKMRNNIRRVQQDVTLAAGGSGRAKKLAALNRFKDREANFAKTYNHQISRRIVEIAKNNGCSTIVLEDIGNIYDVDNIVLSNWGYYQLHQQIEYKAQMQDINVVKSQLVAEKQMQTMLETIELDKRDEFIAKSLSQIDIKQKKK

SEQ ID NO: 600 - protein, 479 aa, unidentified
  source 1..479: note = Uncultivated organism; MG119-262 effector
MYITRKIELWVKEDDKNLRNEIWRTIRDYEHMVFKSANLIVTNQLFNQTFTDRIVLTDEELSVKREKIERDIDKLNEKLKDEKDDTKKDKLKENRNKLYRQINQLSTEARKMAEEFYTTSEKNTTYQLIRKEFPTLPSHISASLNDMVTKNYSNEIFDVRLGKRSLRTYRKGMPIPFMKLSLKLKKVDDEICLKWVNNIEFTLHFGRDKSNNQIIVERILKGDYKVGDSSIQLKKGKIFILLVVDIPKEENQLNDEVSVGVDLGLSIPAVCSLNVGKERLFIGSYNDFIRVRTQLQSRKRRLQRSLTLTKGGKGRGKKLKAFDRLKTKERNFVKTYNHTLTNRIVKFAKDNLAATIKLEFLEGYGEDETNSFVLRNWSYYELQTQLQYKAEREGIETVFIDPYHTSQICSFCNHYEQGQRLKQSEFLCKNVDCSNKDKKGENKIINADWNASVNIAKSVKFVTKKSDCEYFKRKKDEKT

SEQ ID NO: 601 - protein, 486 aa, unidentified
  source 1..486: note = Uncultivated organism; MG119-263 effector
MPTEYTCITRKIEVHLHKNGDDAESIALLKEQYAIWNAINDNLYKAANRIVSHCFFNDAYEFRLRLHSPEFDKIAKALKDAEKNKLSKKEIDALKKERKKLFDGFKKQRKLFLSGGVDDGKNKEQNSTYKVIREEFSDVIPSKVANCLNQKVVATYKAFTKDVERGTRTIPNYKKGLPVPFQMTLDKELQLKQRSDGSYYVKFPKQLEWDLYFGRDRSNNLEIVKRILSGEYLVGDSSIQQTKRGKIFLLLVVKIPKKEIALDKNIVVGVDLGINVPLYAALNNNVYGGMGVGSRDQFLKVRMQMYARKREMQRCLRYGTNGGHGRKQKLQALERLEGKERNWVHLQNHIFSKALIDYALKNNAGVIQMEKLSGFGKDKDGEVDDGYKFILRNWSYFELQNMIEYKANAVGIEVRYVDPYHTSQTCSFCGHYEPGQRISQSQFECRNPDCQKGKGKKRSNGEYEGINADWNAARNIALSDKIVSEK

SEQ ID NO: 602 - protein, 440 aa, unidentified
  source 1..440: note = Uncultivated organism; MG119-264 effector
MNITKKIKIEMCAPQDFESLDKEFNYRCQKSQSISLGKTRRDVENAFVEMNSVLWRVYNKVSFKCGTYIYLDKDWGEVSGMNNELYVFTNEVMNEVKKECPSINSVPSSIILRLINDVASKIGSGESKKEIKRGDRKTPEFNKGVPMGIGNKQLRFYKKHNGNEYYVRIYGIWFKTILSKDHQGNRMIINHIMNGDEGYKASDSTLSVKEKGKNIDFILNLSVKIPESKELYDRGIICGIDLGMKNLAVCTICDKSTGDVITNEYTKKELIEKIDDVSIIEVRNKYKAERERIRKKCELTGISGHGRKAKMNRYYDFGHKEKNVARNIYHNVSRKIIKFCKGNNVGVIHMENLKGYGKKECMAASLWGYYELQQMIEYKAKKDGIDVVYVNPKNTSKTCSECGHVNENLALKDRDWICPHCGKHHDRDINAAINIARMLK

SEQ ID NO: 603 - protein, 464 aa, unidentified; identical to SEQ ID NO: 476
  source 1..464: note = Uncultivated organism; MG119-265 effector

SEQ ID NO: 604 - protein, 464 aa, unidentified
  source 1..464: note = Uncultivated organism; MG119-266 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGKDKSNNASIIEKTISGEYKFCDSNVVVEKNIMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 605 - protein, 488 aa, unidentified
  source 1..488: note = Uncultivated organism; MG119-267 effector
MATEYTCITRKIEVHLHKHGDSEEAAQRFKDEYRIWDDINNNLYKAANRIISHCFFNDTYEYRLKLHSPRLQEIEKLLSNPKRNKLSDEDVKQLKAERKHLFADFKKQRQVFLRGGVEDGANPEQNSTYKVVSNEFIDFIPSEVLTNLNQNISSTYREYSLDVERGVRTIPNYKKGIPVPFSIKQKGEIVLKKREDGSIYVRFPKGLEWDLSFGRDRSNNREIVERVLSGQYDVGNSSIQETKNKKRFLLLVVKVPKQVASFDPNRIVGVDLGINVPLYAALNDNEYGGMSIGSRDQFLKVRMRMAAQKRELQHNLRHTTNGGHGRPQKLQALNRLEGKERNWVHLQNHIFSKSIIEYAVRNNAGVIQMERLTGFGRDKNDEVGEDFKFLLRYWSFFELQSMIEYKAKAVGVEVRYINPYHTSQTCSFCGHYEKGQRINQATFICKNPECTKGKGKQRTDGTFEGINADWNAARNIAFSTEFVDKKKK

SEQ ID NO: 606 - protein, 488 aa, unidentified
  source 1..488: note = Uncultivated organism; MG119-268 effector
MANEFTCITRKIEVHLHKHGDSDEAIQRYKEEYRMWDDINNNLYKAANRIVSHCFFNDAYEYRLKLHSPRFQKIEKLLSNPKRNKLSNDDIKELKAERKLLFSDFKSQRQTFLRGGIETGTNQEQNSTYIVIKNEFIACIPSDVLTNLNQNISSTYRKYTLDVERGIRTIPNFKKGIPVPFSIKPNGEIALKKRDDGTIYVRFPKGLEWDLNFGRDRSNNREIVERVLSGQYGVGNSSIQESKNKKQFLLLVVKIPKENRVLDKERIVGVDLGVNTPLYAALNDNEYGGMGIGSREQFLKVRERMNAQKRELQRSLRHSTNGGHGRSQKLQALDRLEGKERNWVHLQNHIFSKSIIEYALKNDAGVIQMERLTGFGRDNNEEVQNEYKYILRYWSYFELQTMIEYKAKAAGIEVRYINPYHTSQTCSFCGHYEKGQRINQSTFICKNPDCTKGKGKQKSDGAYEGINADWNAARNIARSNEFVGKKKK

SEQ ID NO: 607 - protein, 464 aa, unidentified
  source 1..464: note = Uncultivated organism; MG119-269 effector
MAVKAAKIELEVCDVNKRNSFIESVGKHINNTFKSQYILTLKENKKDIYDDLLKLQKSNFRLHNFIVNKYQNFINSDDKKFKLENNEKNEINHLATKYSKKLCEEYQLNNINSTIPNGILNLIDKNVKKTIKDIKNGKRIWQSYREQVPIPLSQKDAIVKINGKHFTKIDEMNKNYYIVFWGILFKTNLGKDKSNNASIIEKTISGEYKFCDSNVVVEKNKMFLLLSYDAPQKQIELDDDKVLGIDLGIKIPAVGCIYSKTLGKPITYQHNGKDVIESFDETYVKSKYVNKYEKKEIKPSILTIRNAIKIEHEKRKKDAKYICTNGHGRNRKMKFADKISNKEKNTANLYYHNLSRDIINFALKNKCKQINIENLKGFKGDKVLINGWGYFTLQNQIEYKANEYGIKVKKINPKNTSRTCSSCGHVSEKNRIDQEHFKCEHCGLEMNADYNASINIAKSENFVK

SEQ ID NO: 608 - protein, 481 aa, unidentified
  source 1..481: note = Uncultivated organism; MG119-270 effector
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGNSSIQETKNRKRFLLLVVKIPKESRALIPDRVVGVDLGVNIPLYAALNDNTYGGLSIGSRDQFLKVRMHMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 609 - protein, 481 aa, unidentified
  source 1..481: note = Uncultivated organism; MG119-271 effector
MATEYTCITRKIEVHLHRHGDSEEAKQRLAEEYQMWDEINNNLYKAANRIISHCFFNDAYEYRLKLHSPRFKEIEDLLKNAKRKKLTKEDIDELKAERKRLFAEFKKQRQEFLGGSEQNSTYKVATQEFLDKIPSNVLTCLNQNIAGVYREFSLEVERGQRTIPNYKKGIPVPFPLKVHGELMLMRRSDNSVYVRFPKGLEWDLSFGRDRSNNREIVERVLDGRYDVGNSSIQESKNGKRFLLLVVKIPKENRALDANRVVGVDLGVAVPLSAALNDNEYGGMSIGSYDQFTKMRMRMSAQKRELQRNLRHTTNGGHGRAQKLQALERLEGKERNWVHLQNHIFSKSVIEFAIQNGAGAIQMERLAGFGRDKNNEVDEGYKFVLRYWSFFELQSMIEYKAKAAGIEVRYVDPYHTSQTCSFCGHYEKGQRISQSKFVCKCPECTKGKGKQRADGTFEGINADWNAARNIALSTKIVDRKKK

SEQ ID NO: 610 - protein, 481 aa, unidentified
  source 1..481: note = Uncultivated organism; MG119-272 effector
MATEYTCITRKIEVHLHRHGDSEEAAQRLKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSDVLTCLNQNISSTYREYALDVESGRRTIPNFKKGIPVPFAIKVHRELALRKREDGSIYIRFPKGLEWDLNFGRDRSNNREIVERVLSGQYDVGNSSIQETKNRKRFLLLVVKIPKESRALNPDRVVGVDLGVNIPLYAALNDNTYGGLSIGSRDQFLKVRMHMAAQKRELQRNLRVATNGGHGRKQKLQALDRLEGKERNWVHLQNHIFSKSIIEYALRNEAGAIQMERLTGFGHDRNDEVDEGFKFILRYWSFFELQTMIEYKAKAAGIEVRYVDPYHTSQTCSFCGHYEKGQRVNQATFICKNPDCTKGKGKERSDGTFEGINADWNAARNIALSDKIVERKKK

SEQ ID NO: 611 - protein, 481 aa, unidentified
  source 1..481: note = Uncultivated organism; MG119-273 effector
MANEFVCITRKIEVYLHKHGDDDESKLRYDAEREKWRAINDNLYKAANYITTHCFFNDAFEERLRVQSPEYNRIEKLLRRAKKDNLSYAEIRVLKEERKKLQIQFKQQKKEFLQTSEQNSTYRMVSEEFGDIISSEILTCLNQNISSTYSCYRKEIEYGKRTIPNYKRGIPVPFSIKKKQQLNIKRRADGSIFVRLPFGMEWDLFFGRDRSNNREIVDRVLEQKYDVGNSSLQETQKGKTFLLLVVKIPKQIQELNFNRVVGVDLGINVPLYAALNDNEYGGLSIGSREQFLNVRMRLTKQKRELQYALRNSTNGGHGRKQKLQALERVEKKERNWVHLQNHIFSKSIIEYALKHSAGVIQMENLTGFGRDVDNEVEEDYKFILRYWSFYELQQLIEYKANAAGITVRYINPYHTSQRCSFCGHYEQGQRINQSTFICKNNECKKGKGKTKSDGTYLGINADWNAARNIALSVDFVDKRKK

SEQ ID NO: 612 - protein, 489 aa, unidentified
  source 1..489: note = Uncultivated organism; MG119-274 effector
MANTEFTCITRKIEVHLHRHGDSDEAALRLKNEYHFWDEINDNLYKAANRIISHCFFNDTYEYRLKLHSPRFQEIEKLLKNAKRNKLTDEEIKELKAERKLLFSDFKKQRYTFLRGGIEDGANPEQNSTYRVVSNEFIDTIPSEVLTNLNQNISSTYKNYTIDVERGLRTIPNYKRGMPVPFSIKKHGVLALRKREDGSIYVAFPKGLEWDLSFGRDRSNNREIVERVLSGLYDVGNSSIQEAKNGKRFLLLVVKIPKEVKILDTNRVVGVDLGVAVPLYAAISDNEYGGMSIGSYDQFIKVRMRMNAQKREMQHNLRHTTNGGHGRTQKLQALERLEGKERNWVHLQNHIFSKSIIEYALQNNAGAIQMERLTGFGHDKNNEVDEGYKFILRYWSFFELQTMIEYKAKAAGIEVRYIDPYHTSQTCSFCGHYEKGQRLNQSKFVCKNPDCVKGKGKQMSDGSFEGINADWNAARNIALSTKIVDRKKK

SEQ ID NO: 613 - protein, 488 aa, unidentified
  source 1..488: note = Uncultivated organism; MG119-275 effector
MATEYTCITRKIEVHLHRHGEDEEAKQRLIDDYRVWDTINDNLYKAANRIVSHCFFNDAFEYRLKIHSPRFQEIEKLLKYSKRNKLTDEDIKQLKAERKQMFADFKKQRHTFLRGGVAEGANPEQNSTYKVISNEFLEVIPSEILTNLNQNISSTYKNYSLDVERGIRTIPNYKRGIPVPFSIKQRGELMLKRRDDGSIFIRFPMGLEWDLSFGRDRSNNREIVERVLSGQYDVGNSSIQESKNRKRFLLLVVKIPKESRNLNPDRIVGVDLGINIPLYAALNDNDYGGMGIGSREQFLNMRMRMAAKKRELQRNLRQSTNGGHGRKQKLQALERLEGKERNWVHLQNHIFSKSIIEYALKNNAGAIQMERLTGFGRDKNDEVDSNFKFILRYWSFFELQTMIEYKANAAGIEVRYVDPYHTSQTCSFCGHYEKGQRLNQSTFVCKNPDCEKGKGKKLSDGTYQGINADWNAARNIALSDKIVDRKKK

SEQ ID NO: 614 - protein, 488 aa, unidentified
  source 1..488: note = Uncultivated organism; MG119-276 effector
MATEYTCITRKIEVHLHKHGEGEEATKRLQEEYRIWDDINNNLYKAANRIVSHCFFNDTYEYRLKLHSPRFQEIEKKLSNPKRNKLSDEEVKQLKAERKLLFADFKKQRQVFLRGGIEEGANPEQNSTYKVVSKEFIDTIPSEVLTNLNQNISSTYREYALDVERGVRTIPNYKKGIPVPFSIKQKGDIVLKKRDDGTIYVRFPKGLEWDLSFGRDRSNNREIVERVLSGEYDVGNSTIQETKNRKRFLLLVVKIPKQTVVFNPNRIVGVDLGINVPLYAALNDNEYGGMGIGSRDQFLKVRMRMVAQKRELQRNLRHTTNGGHGRTQKLQALDRLEGKERNWVHLQNHIFSKSIIEYAVKNNAGIIQMERLTGFGRDDKEEVQDEYKFILRYWSFFELQTMIEYKAKAAGIEVRYINPYHTSQTCSFCGHYEKGQRINQGTFICKNPECVKGKGKQKSDGSYEGINADWNAARNIAKSTEVVDKKRK

SEQ ID NO: 615 - protein, 486 aa, unidentified
  source 1..486: note = Uncultivated organism; MG119-277 effector
MAAEFTCITRKIEVHLHRHGEGEEAEMRLKEEYRIWDEINDNLYKAANRVVSHCFFNDSYEYRLKLHSPRFQEIEKLLKNAKRNKLSNDDIRELKEERSLLFSDFKKQRLAFLQGNSGKGSEQNSTYKVISNEFVDVIPSEVLTNLNQDISSTYKNYALEVERGARTIPNYKKGIPVPFSIKQSGELRLRKREDGSVYITFPRGLEWDLNFGRDRSNNREIVERVLSGLYDVGNSSIQETKSRKRFLLLVVKIPKEASKLDKNKVVGVDLGVAVPLYAALNDNQYGGMSIGSYEQFTKVRQRMFAQKKELQRNLRHTTNGGHGRKQKLQALERMEGKERNWVHLQNHIFSKSLIEYALGNKAGVVQMERLTGLGRDVNDELGEGYKFVLRYWSFFELQTMIEYKAKAAGIEVRYINPYHTSQTCSFCGHYEKGQRINQSTFICKNPDCIKGKGERKSDGSYKGINADWNAARNIALSKEYVEKKKK

SEQ ID NO: 616 - protein, 466 aa, unidentified
  source 1..466: note = Uncultivated organism; MG119-278 effector
MDWDKLSLHSKEEYMILTRKLALKARKKEDLGYLYTLNNSVFKAYNTVVNTLYFSYLFKDKITLTDAELAKRREKISRELVKIRGKISLIDKDDKTKLDNLKKRRKGFYNKLNTLTREARDRAEEYYTTSEKNYGYQIISKEFPDIPSYIRACINVEVYANFTKDINRIRNGEISVRTYRKGTPIPFMASAGKFIKDEDNIIFKWLKGIEFDIQFGRDKSARKSEVEKIISGDLKMSDSKIQIKNRKIFLLLAIDQPVEKKDLNMNITVGVDVGIKYPAYVSTNSGIQHKHIGSIDDLFKLRMQIQKRRQRLQKQIKSAKGGKGRGKKLKALNRMREKERNYVRTYNHYISREVVNFALENRAGTIIMEDLSGFGVNYKKKNTILRNWSYFELQSMIDYKAKLMGIKVEKIKPEYTSQTCALCGMKGKREEREIFICLNSECKNFEKPINADYNAAMNISRRGIKK

SEQ ID NO: 617 - protein, 453 aa, unidentified
  source 1..453: note = Uncultivated organism; MG119-279 effector
MILTRKLKLELIDRDNLEYLYNLNYLTFKAYNTAVNALYFSSLFKDIIFDSDKEIVEKRKEIIQQLKSIDEEIFQAKDEDKENKDELKRQKTYLQSMYKSLFRDVQKTAEEFYGGSERNLAYKIINKDYSELPSYIKAAIGADVYANYKSDIGQIRRGEISLRTYRKGIPIPFMKSAGKFIKEDDNIFFQWVKGIKFKIIFGRDKSARQVEVERILACKLKISDSKIQFKNKKIFLLLAINQSIDKKELDKNIAVGVDLGIKYPAYVSTNSGIQHKHIGSYDDLFKLRLQIQERKKRLQKNLKYASGGKGRKKKLKALNRIETKERNYIRTYNHSISKEIVNFTLQNKAANIIIEDLSGFGSRDDNKKRVLRNWSYFELQSMIKYKAELKGIKVIEISPKYTSQTCAICGERGDRDTQNSFVCSNPECENFNQSVNADYNASFYIAKTGLEKK

SEQ ID NO: 618 - protein, 453 aa, unidentified
  source 1..453: note = Uncultivated organism; MG119-280 effector
MILTRKLKLELIDRDNLEYLYNLNYLTFKAYNTAVNALYFSSLFKDIIFDSDKEIVEKRKEIIQQLKSIDEEIFQAKDEDKENKDELKRQKTYLQSMYKSLFRDVQKTAEEFYGGSERNLAYKIINKDYSELPSYIKAAIGADVYANYKSVIGQIRRGEISLRTYRKGIPIPFMKSAGKFIKEDDNIFFQWVKGIKFKIIFGRDKSARQVEVERILACKLKISDSKIQFKNKKIFLLLAINQSIDKKELDKNIAVGVDLGIKYPAYVSTNSGIQHKHIGSYDDLFKLRLQIQERKKRLQKNLKYASGGKGRKKKLKALNRIETKERNYIRTYNHSISKEIVNFTLQNKAANIIIEDLSGFGSRDDNKKRVLRNWSYFELQSMIKYKAELKGIKVIEISPKYTSQTCAICGERGDRDTQNSFVCSNPECENFNQSVNADYNASFYIAKTGLEKK

SEQ ID NO: 619 - protein, 453 aa, unidentified
  source 1..453: note = Uncultivated organism; MG119-281 effector
MILTRKLQLKIIDVKNWLFLNELNNNVFKAYNDVVNTHYFSTLFKDKIALTDEEINSKTIKINKKLNKNKEIIKEAKDDKKKIETLKKEQQKLYNTLRILNREARDKAEVFYTTGDKNFGYQMLSRKFPEIPSTIRTCINADVYANFTKEINDIRSGKRAVRTYRKGVPIPFQLDKSKEIFVQKNDKIIFRWIKDIDFEIIFGRDKSARQSEVEKIISGDYKMSDSKLQIKNKKIFLLLALNQPEEKLQLDKSVTIGVDLGIVFPAYVSTNSGIQHQRIGSSDDLFKLRMQIQKRRRRLQKDIKSAHGGKGRKKKLKALERLRQKERNYVRTYNHFISREIINFAIQQKAATIFIEDLSGFGKNKEKGNIILRNWSYFELQSMIEYKAKLKGVEVKKVDPKYTSQICSICGEKGKRESQKDFLCLNKKCENFGKKILADYNAAWNIAKGGIKI

SEQ ID NO: 620 - protein, 458 aa, unidentified
  source 1..458: note = Uncultivated organism; MG119-282 effector
MNIARKIKMEIISPSRSEEILKKYNINRKEKMMISSGQSIREIEQSLLDMNEILPRACNMTMVDFHKIIHSNLSDDEKCTLIKNRGIIGKKPGEKVNNDPNDIRVHLKDNLNLLASEYGSINNVPSSIYVRGYTDMTNGIIEKSGKIKREILRYDRVLPEFKKGMPMGLSSQRIRIYPNEDGEFYFVIWGVLFKTILRKDKQKNRDILYEIINNNDYKLSDSTLSVFKNGKNTEIYFNMTVKIPESKELYDRGIICGLDLGMKTLVTCAVCDKATGNVITDEHSKKEIIGKIGDGSTIIESRNKYKIEGERIRKKCEMMGVNSHGRNAKMEEYYKYIHNKERNAAKNIYHTVSRKVIKLCKEYNAGVIQMENLEGYGKKECVAASLWGYYELQKMIEYKANKEGIEVKYVNPKNTSKTCSECHGVNESLTLKDREWVCPHCGKHHDRDVNAAINIARM

SEQ ID NO: 621 - protein, 458 aa, unidentified
  source 1..458: note = Uncultivated organism; MG119-283 effector
MNIVRKIKMEIISPSRSEEILKKYNINRKEKMMISTGQSIREIEQSLLDMNEILPRACNMTMVDFHKIIHSNLSDDEKCALIKNRGIIGKKPGEKVNNDPNDIRVHLKDNLNLLASEYGSINNVPSSIYVRGYTDMTNGIIEKSGKIKREILRYDRVLPEFKKGMPMGLSSQQIRIYPNEDGEFYFVIWGVLFKTILRKDKQKNRDILYGIINNNDYKLSDSTLSVFKNGKNTEIYFNMTVKIPESKELYDRGIICGLDLGMKTLVTYAICDKATGNVITDEHSKKEIIGKIGDGSTIIEFRNKYKIEGERIRKKCEMMGVNSHGRNAKMEEYYKYIHNKERNAAKNIYHTVSRKVIKLCKENNAGVIQMENLEGYGKKECIAASLWGYYELQKMIEYKAKKEGIEVKYVNPKNTSKTCSECHGVNESLTLKDREWVCPHCGKHHDRDVNAAINIARM

SEQ ID NO: 622 - protein, 458 aa, unidentified
  source 1..458: note = Uncultivated organism; MG119-284 effector
MNIVRKIKMEIISPSRSEEILKKYNINRKEKMMISTGQSIREIEQSLLDMNEILPRACNMTMVDFHKIIHSNLSDDEKCALIKNRGIIGKKPGEKVNNDPNDIRVHLKDNLNLLASEYGSINNVPSSIYVRGYTDMTNGIIEKSGKIKREILRYDRVLPEFKKGMPMGLSSQQIRIYPNEDGEFYFVIWGVLFKTILRKDKQKNRDILYEIINNNDYKLSDSTLSVFKNGKNTEIYFNMTVKIPESKELYDRGIICGLDLGMKTLVTCAVCDKATGNVITDEHSKKEIIGKIGDGSTIIESRNKYKIEGERIRKKCEMMGVNSHGRNAKMEEYYKYIHNKERNAAKNIYHTVSRKVIKLCKENNAGVIQMENLEGYGKKECVAASLWGYYELQKMIEYKAKKEGIEVKYVNPKNTSKTCSECHGVNESLTLKDREWVCPHCGKHHDRDVNAAINIARM

SEQ ID NO: 623 - protein, 486 aa, unidentified
  source 1..486: note = Uncultivated organism; MG119-285 effector
MADKQKEVIITRKIEVHLHHDPVADPDKVEYNRHWEVWHTINNNLYKAANRIVSHKFFNDEYEHRLQLHSPEFQEINRKLARAKKEQLSAEEIAALRSRIKELYAQFANEKRTFLRGGVAVGPNPEQNSTDKVVRDEFSDVVPTEILTCLNQSVSSTYSEYKKQIARGERAIPNYKKGMPVPIRMKANGQLKLRRRPDNSIYVLTPRGLEWDLSFGRDRSNNREIVERVLSGQYDVGGSSIQEKGRKIFLLLVVKLPASQNELNPERVVGVDLGLNIPLYAALNDNEYGGLSIGSRDKFMKERERMSARRRELQRSLRISTQGGHGRSHKLKALDQFEEKERNWVHTQNHIFSRQVVEYAKSNGAGVIQMESLKGSGKDKEEHIEQGFKYVVRYWSYFELQTLIQQKAEREGIEVRKVNPYHTSQTCSFCGHYEKGQRVSQSTFICKNPACAHGKGKQLKDGTFAGINADWNAARNIALSTEWVKE

SEQ ID NO: 624 - protein, 481 aa, unidentified
  source 1..481: note = Uncultivated organism; MG119-286 effector
MATEYTCITRKIEVHLHRHGDSEEALQRYKEEFRIWDEINDNLYKAANRIISHCFFNDAYEYRLKLHSPRFQEIEKLLKYAKRNKLTDDDIKALKAERKELFAEFKRQRQSFLGGSEQNSTYKVVTDEFLEVIPSHVLTCLNQNISSTYREYALDVEHGRRTIPNFKKGIPVPFPIKATGELLLRKREDGSIYIRFPKGLEWDLNFGRDRSNNREIVERVLSGQYDVGNSSIQETKNRKRFLLLVVKIPKESRTLNPDRVVGVDLGVNIPLYAALNDNTYGGLSIGSRDQFLKVRMRMAAQKRELHRNLRVATNGGHGRKQKLQALDRLEGKERNWVHLQNHIFSKSIIEYALRNEAGAIQMERLTGFGHDRNDEVDEGFKFILRYWSFFELQSMIEYKAKAAGIEVRYVDPYHTSQTCSFCGHYEKGQRVNQATFICKNPDCTKGKGKEHSDGTFEGINADWNAARNIALSDKIVERKKK

SEQ ID NO: 625 - RNA, 172 nt, synthetic construct
  source 1..172: mol_type = other RNA; note = 119-28 sgRNA1_Mouse_Alb
  modified_base 1: mod_base = um
  modified_base 2: mod_base = um
  modified_base 3: mod_base = gm
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 170: mod_base = cm
  modified_base 171: mod_base = um
  modified_base 172: mod_base = OTHER; note = 2'-O-Methyladenosine
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacaagattgatgaagacaacta

SEQ ID NO: 626 - RNA, 172 nt, synthetic construct
  source 1..172: mol_type = other RNA; note = 119-28 sgRNA2_Mouse_Alb
  modified_base 1: mod_base = um
  modified_base 2: mod_base = um
  modified_base 3: mod_base = gm
  modified_base 170: mod_base = OTHER; note = 2'-O-Methyladenosine
  modified_base 171: mod_base = cm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacggtcagtgaagagaagaaca

SEQ ID NO: 627 - RNA, 172 nt, synthetic construct
  source 1..172: mol_type = other RNA; note = 119-28 sgRNA3_Mouse_Alb
  modified_base 1: mod_base = um
  modified_base 2: mod_base = um
  modified_base 3: mod_base = um
  modified_base 170: mod_base = cm
  modified_base 171: mod_base = cm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaacagtgtagcagagaggaacca

SEQ ID NO: 628 - RNA, 172 nt, synthetic construct
  source 1..172: mol_type = other RNA; note = 119-28 sgRNA4_Mouse_Alb
  modified_base 1: mod_base = um
  modified_base 2: mod_base = um
  modified_base 3: mod_base = gm
  modified_base 170: mod_base = OTHER; note = 2'-O-Methyladenosine
  modified_base 171: mod_base = gm
  modified_base 172: mod_base = OTHER; note = 2'-O-Methyladenosine
  modified_base 1^2: mod_base = OTHER; note = Phosphorothioate bond
  modified_base 2^3: mod_base = OTHER; note = Phosphorothioate bond
  modified_base 3^4: mod_base = OTHER; note = Phosphorothioate bond
  modified_base 169^170: mod_base = OTHER; note = Phosphorothioate bond
  modified_base 170^171: mod_base = OTHER; note = Phosphorothioate bond
  modified_base 171^172: mod_base = OTHER; note = Phosphorothioate bond
ttgaaataaaatgaatttcaaaccccttcgggggagggcgcgttggagcgccttagtttgaggtgcagaatcaaaaaaactgcgacgatggaggtcgtttcagtctctgtacactcaaaaaattcacttgagaaatcaagtgaatatccaactctgtggaaacagggagaga

(19) United States
(12) Patent Application Publication
(10) Pub. No.: US 2026/0275332 A1
(43) Pub. Date: Sep. 17, 2026

(54) CLASS 2, TYPE V CRISPR SYSTEMS

(71) Applicant: Metagenomi Therapeutics, Inc., Emeryville, CA (US)

(72) Inventors: Brian C. Thomas, Berkeley, CA (US); Lisa Alexander, Albany, CA (US); Molly Brothers, Oakland, CA (US); Christopher Brown, Albany, CA (US); Dominic Castanzo, Oakland, CA (US); Cindy Castelle, San Francisco, CA (US); Daniela S.A. Goltsman, Oakland, CA (US); Liliana Gonzalez-Osorio, Emeryville, CA (US); Paula B. Matheus Carnevali, Pinole, CA (US)

(21) Appl. No.: 19/162,390

(22) PCT Filed: Mar. 8, 2024
(86) PCT No.: PCT/US2024/019205

§ 371 Date: Sep. 4, 2025

Related U.S. Application Data
(60) Provisional application No. 63/489,162, filed on Mar. 8, 2023.
(60) Provisional application No. 63/504,422, filed on May 25, 2023.
(60) Provisional application No. 63/587,655, filed on Oct. 3, 2023.

(51) Int. Cl. C12N 15/11; C07K 14/76; C07K 14/775; C12N 9/10; C12N 9/22

(52) U.S. Cl. CPC C12N 15/111; C07K 14/76; C07K 14/775; C12N 9/1007; C12N 9/226; C07K 2319/80; C12N 2310/20; C12Y 201/01037

(57) ABSTRACT

Described herein are methods, compositions, and systems derived from uncultivated microorganisms useful for gene editing.

### CROSS-REFERENCE

This application claims the benefit of and priority to U.S. Provisional Patent Application No. 63/489,162, filed Mar. 8, 2023, U.S. Provisional Patent Application No. 63/504,422 filed May 25, 2023, and U.S. Provisional Patent Application No. 63/587,655 filed Oct. 3, 2023, each of which is incorporated by reference in its entirety herein.

### SUMMARY

Described herein, in certain embodiments, are engineered nuclease systems comprising: a) an endonuclease comprising a sequence having at least 80% sequence identity to any one of SEQ ID NOs: 1-325, 420-431, 476-624, 629, 1065-1090, 1114-1118, and 1746-1752; and b) an engineered guide polynucleotide configured to form a complex with the endonuclease and to hybridize to a target nucleic acid sequence. In some embodiments, the endonuclease comprises a sequence having at least 90% sequence identity to any one of SEQ ID NOs: 1-325, 420-431, 476-624, 629, 1065-1090, 1114-1118, and 1746-1752. In some embodiments, the endonuclease comprises a sequence having 100% sequence identity to any one of SEQ ID NOs: 1-325, 420-431, 476-624, 629, 1065-1090, 1114-1118, and 1746-1752. In some embodiments, the engineered guide polynucleotide comprises a crRNA and a tracrRNA. In some embodiments, the engineered guide polynucleotide comprises a sequence having at least 90% sequence identity to any one of SEQ ID NOs: 333-335, 355-357, 410-411, 346-347, 368-369, 412-413, 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1091-1113, 1119-1120, 1697-1731, and 1876. In some embodiments, the engineered guide polynucleotide comprises a sequence having 100% sequence identity to any one of SEQ ID NOs: 333-335, 355-357, 410-411, 346-347, 368-369, 412-413, 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1091-1113, 1119-1120, 1697-1731, and 1876. In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the endonuclease binds non-covalently to the engineered guide polynucleotide. In some embodiments, the endonuclease is covalently linked to the engineered guide polynucleotide. In some embodiments, the endonuclease is fused to the engineered guide polynucleotide. In some embodiments, the endonuclease system further comprises a DNA methyltransferase. In some embodiments, the DNA methyltransferase binds non-covalently to the endonuclease. In some embodiments, the DNA methyltransferase is fused to the endonuclease in a single polypeptide. In some embodiments, the DNA methyltransferase comprises Dmn13A or Dnmt3L.

Described herein, in certain embodiments, are engineered nuclease systems comprising: a) an endonuclease comprising a sequence having at least 80% sequence identity to any one of SEQ ID NOs: 6-14; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 333-335 and 355-357.

Described herein, in certain embodiments, are engineered nuclease systems comprising: a) an endonuclease comprising a sequence having at least 80% sequence identity to ID NO: 15; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 410-411.

Described herein, in certain embodiments, are engineered nuclease systems comprising: a) an endonuclease comprising a sequence having at least 80% sequence identity to any one of SEQ ID NOs: 16-29; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 346-347, 368-369, and 412-413.

Described herein, in certain embodiments, are engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876.

Described herein, in certain embodiments, are engineered nuclease systems comprising: a) an endonuclease comprising a sequence having at least 80% sequence identity to any one of SEQ ID NOs: 1065-1090 1114-1118, and 1746-1752; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1091-1113, 1119-1120, and 1876. In some embodiments, the engineered guide polynucleotide is a single guide nucleic acid. In some embodiments, the engineered guide polynucleotide is a dual guide nucleic acid. In some embodiments, the engineered guide polynucleotide is RNA. In some embodiments, the endonuclease binds non-covalently to the engineered guide polynucleotide. In some embodiments, the endonuclease is covalently linked to the engineered guide polynucleotide. In some embodiments, the endonuclease is fused to the engineered guide polynucleotide. In some embodiments, the endonuclease system further comprises a DNA methyltransferase. In some embodiments, the DNA methyltransferase binds non-covalently to the endonuclease. In some embodiments, the DNA methyltransferase is fused to the endonuclease in a single polypeptide. In some embodiments, the DNA methyltransferase comprises Dmnt3A or Dnmt3L.

Described herein, in certain embodiments, are methods for modifying a target nucleic acid sequence comprising contacting the target nucleic acid sequence using the engineered nuclease system described herein. In some embodiments, modifying the target nucleic acid sequence comprises binding, nicking, or cleaving the target nucleic acid sequence. In some embodiments, the target nucleic acid sequence comprises genomic DNA, viral DNA, viral RNA, or bacterial DNA. In some embodiments, the modification is in vitro. In some embodiments, the modification is in vivo. In some embodiments, the modification is ex vivo.

Described herein, in certain embodiments, are methods of modifying a target nucleic acid sequence in a mammalian cell comprising contacting the mammalian cell using the engineered nuclease system described herein. In some embodiments, the methods further comprise selecting cells comprising the modification.

Described herein, in certain embodiments, are methods of modifying TRAC comprising contacting TRAC using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 767-798. In some embodiments, the target nucleic acid sequence comprises a sequence having any one of SEQ ID NOs: 799-830.

Described herein, in certain embodiments, are methods of modifying APOA1 comprising contacting APOA1 using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 831-904, 979-1021, 1219-1237, 1491-1506, and 1663-1669. In some embodiments, the target nucleic acid sequence comprises a sequence having any one of SEQ ID NOs: 905-978, 1022-1064, 1238-1256, and 1670-1676.

Described herein, in certain embodiments, are methods of modifying AAVS1 comprising contacting APOA1 using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1257-1324, 1523-1562, 1677-1686, and 1753-1779. In some embodiments, the target nucleic acid sequence comprises a sequence having any one of SEQ ID NOs: 1325-1392, 1563-1602, 1687-1696, and 1780-1806.

Described herein, in certain embodiments, are methods method of modifying albumin comprising contacting albumin using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1121-1169, 1393-1441, 1603-1632, 1887, 1889, 1891, and 1892-1893. In some embodiments, the target nucleic acid sequence comprises a sequence having any one of SEQ ID NOs: 1170-1218, 1442-1490, 1633-1662, 1888, 1890, and 1994.

Described herein, in certain embodiments, are nucleic acids encoding the engineered nuclease system described herein.

Described herein, in certain embodiments, are cells comprising the engineered nuclease system described herein. In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, primary cell, or a derivative thereof. In some embodiments, the cell is an engineered cell. In some embodiments, the cell is a stable cell. In some embodiments, the cell is a primary cell. In some embodiments, the primary cell is a T cell. In some embodiments, the primary cell is a hematopoietic stem cell (HSC).

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts typical organizations of CRISPR/Cas loci of different classes and types that were previously described before this disclosure.

FIGS. 2A-2D depict an overview of the MG119 Family. FIG. 2A depicts a multiple alignment of MG119 effectors representatives showing domains compositions and conservation of the RuvC catalytic residues critical for function for a double-stranded DNA cleavage activity. FIG. 2A discloses MG119-3 (SEQ ID NO: 32), MG119-4 (SEQ ID NO: 33), MG119-(SEQ ID NO: 31), MG119-1 (SEQ ID NO: 30), and MG119-5 (SEQ ID NO: 34). FIG. 2B depicts a representation of a CRISPR-containing contig with genomic context surrounding the CRISPR array and the Cas effector (example of MG119-1). FIG. 2C depicts folding of the Direct repeat of MG119-1 (SEQ ID NO: 1934). FIG. 2D depicts a single guide RNA designed for MG119-1 (SEQ ID NO: 1935).

FIGS. 3A-3C depict an overview of the MG90 Family. FIG. 3A depicts a multiple alignment of MG90 effectors representatives showing domains compositions and conservation of the RuvC catalytic residues critical for function for a double-stranded DNA cleavage activity. FIG. 3A discloses SEQ ID NO: 26 (MG90-21), SEQ ID NO: 20 (MG90-8), SEQ ID NO: 17 (MG90-5), SEQ ID NO: 23 (MG90-18), and SEQ ID NO: 16 (MG90-3).

FIG. 3B depicts a representation of a CRISPR-containing contig with genomic context surrounding the CRISPR array and the Cas effector (example of MG90-5). FIG. 3C depicts folding of the Direct repeat of MG90-5.

FIGS. 4A-4C depict an overview of the MG126 Family. FIG. 4A depicts a multiple alignment of MG126 effectors representatives showing domains compositions and conservation of the RuvC catalytic residues critical for function for a double-stranded DNA cleavage activity. FIG. 4A discloses MG126-3 (SEQ ID NO: 319), MG126-4 (SEQ ID NO: 321), and MG126-7 (SEQ ID NO: 324). FIG. 4B depicts a representation of a CRISPR-containing contig with genomic context surrounding the CRISPR array and the Cas effector (example of MG126-4). FIG. 4C depicts folding of the Direct repeat of MG126-4 (SEQ ID NO: 1936).

FIGS. 5A-5C depict an overview of the MG118 Family. FIG. 5A depicts a multiple alignment of MG118 effectors representatives showing domains compositions and conservation of the RuvC catalytic residues critical for function for a double-stranded DNA cleavage activity. FIG. 5A discloses SEQ ID NO: 15 (MG118-1). FIG. 5B depicts a representation of a CRISPR-containing contig with genomic context surrounding the CRISPR array and the Cas effector (example of MG118-1). FIG. 5C depicts folding of the direct repeat of MG118-1 (SEQ ID NO: 1937).

FIGS. 6A-6C depict an overview of the MG122 Family. FIG. 6A depicts a multiple alignment of MG122 effectors representatives showing domains compositions and conservation of the RuvC catalytic residues critical for function for a double-stranded DNA cleavage activity. FIG. 6A discloses SEQ ID NO: 3 (MG122-3), SEQ ID NO: 4 (MG122-4), and SEQ ID NO: 5 (MG122-5). FIG. 6B depicts a representation of a CRISPR-containing contig with genomic context surrounding the CRISPR array and the Cas effector (example of MG122-4). FIG. 6C depicts folding of the Direct repeat of MG122-4 (SEQ ID NO: 1938).

FIGS. 7A-7C depict an overview of the MG120 Family. FIG. 7A depicts a multiple alignment of MG120 effectors representatives showing domains compositions and conservation of the RuvC catalytic residues critical for function for a double-stranded DNA cleavage activity. FIG. 7A discloses SEQ ID NO: 6 (MG120-1), SEQ ID NO: 7 (MG120-2), SEQ ID NO: 8 (MG120-3), SEQ ID NO: 9 (MG120-4), SEQ ID NO: 10 (MG120-5), SEQ ID NO: 11 (MG120-6), SEQ ID NO: 12 (MG120-7), and SEQ ID NO: 14 (MG120-9). FIG. 7B depicts a representation of a CRISPR-containing contig with genomic context surrounding the CRISPR array and the Cas effector (example of MG120-1). FIG. 7C depicts folding of the Direct repeat of MG120-1 (SEQ ID NO: 1939).

FIGS. 8A-8D depict an overview of the MG91 Family. FIG. 8A depicts a representation of a CRISPR-containing contig with genomic context surrounding the CRISPR array and the Cas effector (example of MG91B-24). FIG. 8B depicts folding of the Direct repeats of MG91B-24 (SEQ ID NO: 1940). FIG. 8C depicts a representation of a CRISPR-containing contig with genomic context surrounding the CRISPR array and the Cas effector (example of MG91C-10). FIG. 8D depicts folding of the Direct repeats of MG91C-10 (SEQ ID NO: 1941).

FIG. 9 depicts in vitro activity of MG119-2 using the TXTL assay. MG119-2 was tested for dsDNA cleavage with two intergenic sequences from the MG119-2 contig, minimal array (MA) sequences containing repeats in the forward or reverse orientation, and a PAM library target plasmid. Positive intergenic enrichment was observed in lane 1 as an amplified cleavage product with intergenic (IG) sequence 1 and the minimal array with repeats in the forward orientation. Lanes 3 and 7 are the negative controls where IGs were omitted, and lane 4 is a third negative control where both the arrays and IGs were omitted.

FIG. 10A depicts a SeqLogo of the MG119-2 PAM (5′-nTnn-3′) determined via next-generation sequencing (NGS) of the cleavage products obtained from the in vitro cleavage assay.

FIG. 10B depicts a histogram of the cutsite (23 bd away from the PAM).

FIGS. 11A and 11B depict examples of active MG119 nuclease and their sgRNA designs. FIG. 11A depicts predicted folding for single guide RNA sequences without spacers. The blue circle represents the first 5′ nucleotide of the tracrRNA and the red circle represents the 3′ nucleotide of the repeat. TracrRNA and repeat sequences are looped with a GAAA tetraloop. The repeat anti-repeat fold is on the 3′ end of each structure. Depicted are three different RNA structures of active guides within the same family. From left to right: the MG119-28 guide has four hairpins, three smaller ones on the 5′ end and a very long hairpin with two bulges next to the repeat anti-repeat fold. The MG119-83 sgRNA has three small hairpins and the repeat anti-repeat has two bulges. The MG119-118 has four hairpins, the second hairpin from the 5′ end branches into three hairpins while the third hairpin and the repeat anti-repeat have one bulge. This guide also has some pairing nucleotides between the 5′ end of the tracr and the 3′ end of the repeat. FIG. 11B depicts in vitro cleavage assay amplification products on 2% agarose gels. Low molecular weight DNA ladders are in lanes 1, 7, and 11. Other lane contents from left to right: (2) MG119-28 nuclease only, MG119-28 nuclease plus (3) sgRNA1 with U67 spacer, (4) sgRNA1 with U40 spacer, (5) sgRNA2 with U67 spacer, and (6) sgRNA2 with U40 spacer; (8) MG119-83 nuclease only, MG119-83 nuclease plus (9) sgRNA1 with U67 spacer and (10) sgRNA1 with U40 spacer; (12) MG119-118 nuclease only, MG119-118 nuclease plus (13) sgRNA1 with U67 spacer and (14) sgRNA1 with U40 spacer. Resulting amplicon products are 188 bp with a U67 spacer carrying guide or 205 bp with a U40 spacer carrying guide.

FIG. 12 depicts sequence logos of protospacer adjacent motifs (PAMs) for active MG119 nucleases.

FIGS. 13A-13F depict example SDS-PAGE gels of protein purification steps and size exclusion chromatography (SEC) A280 traces. FIG. 13A depicts MG119-28A purification with samples recovered (1) post-sonication lysis, (2) post-clarification centrifugation, (3) Ni-NTA gravity column flow-through, (4) eluate from Ni-NTA resin, (5) concentrated sample. FIG. 13B depicts S200i 10/300 GL column SEC A280 trace. Peak fractions were pooled and concentrated. FIGS. 13C and 13D depict MBP-tagged/cleaved MG119-28A purification with samples recovered (1) post-sonication lysis, (2) post-clarification centrifugation, (3) Ni-NTA gravity column flow-through, (4) eluate from Ni-NTA resin, (5) concentrated protein, (6) concentrated protein cleaved overnight with TEV protease, (7) and centrifuged (21,000×g, 4° C., 10 min) to pellet aggregates, (8) Amylose column flow-through, (9) centrifuged flow-through (21,000×g, 4° C., 10 min) to pellet aggregate, and (10) concentrated flow-through. FIG. 13E depicts S200i 10/300 GL column SEC A280 trace. FIG. 13F depicts data demonstrating that of the five MG119 candidates expressed in both the pMGB and pMGBA expression vectors, all showed higher yields in the pMGBA vector.

FIGS. 14A and 14B depict an example of in vitro cleavage efficiency with purified protein. FIG. 14A depicts an agarose gel showing RNP: substrate ratio titration and increasing substrate cleavage at higher ratios. FIG. 14B depicts the percent of substrate cleaved determined for each lane using densitometry. Cleavage fractions were plotted in Prism8, and the slope of the linear range of cleavage was used to calculate protein active fraction. This assay used MG119-28 expressed in the pMGBA backbone.

FIGS. 15A and 15B depict examples of in vitro cleavage and editing efficiency of mouse Hepa1-6 cells DNA. FIG. 15A depicts percent cleavage of MG119-28 with four chemically modified guides targeting the mouse albumin gene at intron 1 (Table 6). Two concentrations of nuclease were tested 15.6 nM (black bars) and 7.8 nM (white bars). Cleavage was normalized to the non-targeting control. MG119-28 can cleave Hepa 1-6 gDNA up to an average of 60% with sgRNA4 at 15.6 nM RNP and up to 33% at 7.8 nM RNP. FIG. 15B depicts percent INDEL generated by MG119-28 in Hepa 1-6 cells normalized to apo reactions. Each condition was performed in triplicate. An average of 25.12% of the sequenced reads were edited with sgRNA3. sgRNA3 is consistently active in vitro and in cells as shown here. The next best guide in cells is sgRNA4 with an average of 4.11% editing. The edits observed are largely a deletion between 4-24 bp.

FIGS. 16A-16B depict the genomic context of a representative Cas nuclease gene in the MG191 family. FIG. 16A depicts a CRISPR array (depicted by repeats and spacers) and nuclease gene (dark grey arrow). FIG. 16B depicts an example of a crRNA fold.

FIGS. 17A-17B depict an example of proteins purified without (MG119-14) and with a fusion protein (SUMO-MG119-1). FIG. 17A: MG119-1 (~56 kDa) can be expressed and purified without a fusion protein, but the resulting sample is relatively low-yield and impure. FIG. 17B: Inclusion of the SUMO domain as an N-terminal fusion protein (SUMO-MG119-1, ~63 kDa) increases both the expression yield and the purity of the sample.

FIGS. 18A-18B depict sequence logos of protospacer adjacent motifs (PAMs) for nuclease MG119-137 from (FIG. 18A) the target strand and (FIG. 18B) the non-target strand.

FIG. 19 depicts an in vitro cleavage assay to determine spacer length preference. Agarose gels with cleavage products including linearized plasmid from dsDNA breaks and nicked plasmid from ssDNA breaks.

FIGS. 20A-20B depict an example of single guide engineering (MG119-2 sgRNA2). FIG. 20A depicts round 1 guide engineering. Truncations to the sequence-optimized WT sgRNA were designed, complexed at a constant ratio of sgRNA:effector, and tested at a constant ratio of RNP:substrate DNA. Cleavage was measured via densitometry and normalized to the extent of cleavage from the non-truncated sequence. In this assay, sgRNA2_4 and sgRNA2_6 are successful truncations (≥80% cleavage relative to the non-truncated sequence). FIG. 20B depicts round 2 guide engineering. Further truncation of the MG119-2 sgRNA2 guide combined the sgRNA2_4 and sgRNA2_6 deletions with additional truncations. The shortest guide to meet the ≥80% relative cleavage threshold is sgRNA2_4.6.11 (96 nt).

FIGS. 21A-21C depict percent indels in K562 cells. Represented is the percentage of amplicons from NGS amplicon sequencing that contain insertions or deletions (indel %). FIG. 21A: Each bar represents the indel % for the indicated MG119-2 guide targeting exon 3 of the human TRAC gene. Black bars represent data from two independent replicates of mRNA+sgRNA nucleofections. Gray bars represent data from one RNP nucleofection. FIG. 21B: Each bar represents the indel % for the indicated MG119-125 guide targeting exon 3 of the human APOA1 gene. Displayed in descending order of indel %. FIG. 21C: Each bar represents the indel % for the indicated MG119-129 guide targeting exon 3 of the human APOA1 gene.

FIGS. 22A-22C depict MG119-2 activity at hAPOA1 exon 3, hALB intron 1, and AAVS1 shown as percent indels in K562 cells. Represented is the percentage of amplicons from NGS amplicon sequencing that contain insertions or deletions (indel %). Black bars represent data from mRNA nucleofections (n=1). FIG. 22A: Each bar represents the indel % for the MG119-2 guide targeting exon 3 of the human APOA gene. FIG. 22B: Each bar represents the indel % for the MG119-2 guide targeting intron 1 of human ALB gene. FIG. 22C: Each bar represents the indel % for the MG119-2 guide targeting the AAVS1 gene.

FIGS. 23A-23C depict MG119-28 activity at hAPOA1 exon 3, hALB intron 1, and AAVS1 shown as percent indels in K562 cells. Represented is the percentage of amplicons from NGS amplicon sequencing that contain insertions or deletions (indel %). Black bars represent data from mRNA nucleofections (n=1). FIG. 23A: Each bar represents the indel % for the indicated MG119-28 guide targeting exon 3 of the human APOA gene. FIG. 23B: Each bar represents the indel % for the indicated MG119-28 guide targeting intron 1 of human ALB gene. FIG. 23C: Each bar represents the indel % for the indicated MG119-28 guide targeting the AAVS1 gene.

FIG. 24 depicts MG119-32 activity at hALB intron 1, hAPOA1 exon 3, and AAVS1 shown as percent indels in K562 cells. Represented is the percentage of amplicons from NGS amplicon sequencing that contain insertions or deletions (indel %). Each bar represents the indel % for the indicated MG119-32 guide targeting intron 1 of human ALB gene, exon 3 of the human APOA gene, and the AAVS1 gene. Black bars represent data from RNP nucleofections (n=2).

FIGS. 25A-25B depict MG119-2 split guide engineering for 119-2_sg2_WT. FIG. 25A: Five split guides for the MG119-2 guide 119-2_sg2_WT were designed and synthesized in two fragments: the “A” fragment (5′ half) and the “B” fragment (3′ half). In some instances (v4 and v5), small portions of the guide were excluded in the final annealed construct. Final designs were mapped to reference (SEQ ID NO: 655) using parent sgRNA. FIG. 25B depicts an agarose gel showing RNP: substrate ratio and the corresponding graph depicts the percent of substrate cleaved by each split guide version as determined for each lane using densitometry.

FIGS. 26A-26B depict MG119-2 split guide engineering for 119-2_sg2_4.6.11. FIG. 26A: Five split guides for the MG119-2 guide 119-2_sg2_4.6.11 were designed and synthesized in two fragments: the “A” fragment (5′ half) and the “B” fragment (3′ half). In some instances (v4 and v5), small portions of the guide were excluded in the final annealed construct. Final designs were mapped to reference (SEQ ID NO: 668) using parent sgRNA. FIG. 26B depicts an agarose gel showing RNP: substrate ratio and the corresponding graph depicts the percent of substrate cleaved by each split guide version as determined for each lane using densitometry. Designs v3 and v4 designs have activity comparable to with sgRNA parent.

FIGS. 27A-27B depict MG119-28 split guide engineering for 119-28_sg1_WT. FIG. 27A: Four split guides for the MG119-28 guide 119-28_sg1_WT were designed and synthesized in two fragments: the “A” fragment (5′ half) and the “B” fragment (3′ half). In some instances (v2 and v3), small portions of the guide were excluded in the final annealed construct. Final designs were mapped to reference (SEQ ID NO: 686) using parent sgRNA. FIG. 27B: depicts an agarose gel showing RNP: substrate ratio and the corresponding graph depicts the percent of substrate cleaved by each split guide version as determined for each lane using densitometry. Activity testing for split 119-28_sg1_WT guides shows split guides v1, v3, and v4 are highly active compared to the sgRNA parent.

FIGS. 28A-28B depict MG119-28 split guide engineering for 119-28_sg1_8.5. FIG. 28A: Four split guides for the MG119-28 guide 119-28_sg1_8.5 were designed and synthesized in two fragments: the “A” fragment (5′ half) and the “B” fragment (3′ half). In some instances (v2 and v3), small portions of the guide were excluded in the final annealed construct. Final designs were mapped to reference (SEQ ID NO: 704) using parent sgRNA. FIG. 28B depicts an agarose gel showing RNP: substrate ratio and the corresponding graph depicts the percent of substrate cleaved by each split guide version as determined for each lane using densitometry. Activity testing for split 119-28_sg1_8.5 guides shows split guides v1, v3, and v4 are highly active compared to the sgRNA parent.

FIGS. 29A-29B depict MG119-32 sgRNA1 round 1 guide length optimization. FIG. 29A: MG119-32 sg1 guide truncation designs, aligned to the untrimmed WT sg1 sgRNA. FIG. 29B depicts relative activity of sg1 guide truncations. The data suggest that designs 5, 6, 7, and 8 have activity better than or comparable to the untrimmed WT sg1 sgRNA.

FIGS. 29C-29D depict MG119-32 sgRNA2 round 1 guide length optimization. FIG. 29C: MG119-32 sg2 guide truncation designs, aligned to the untrimmed WT sg2 sgRNA. FIG. 29D depicts relative activity of sg2 guide truncations. The data suggest that designs 6, 7, and 8 have activity better than or comparable to the untrimmed WT sg2 sgRNA.

FIGS. 30A-30B depict MG119-32 sgRNA1 round 2 guide length optimization. FIG. 30A: Successful guides from round 1 and new round 2 MG119-32 sg1 guide truncation designs, aligned to the untrimmed WT sg1 sgRNA. FIG. 30B depicts relative activity of sg1 guide truncations including successful guides from round 1 (light gray) and new guides from round 2 (dark gray). The data suggest that 119-32_sg1_5.6 and 119-32_sg1_5.7 have activity better than or comparable to the untrimmed WT sg1 sgRNA.

FIGS. 31A-31B depict monitoring nicking activity throughout guide engineering. FIG. 31A: Cleavage reactions are shown for MG119-2 and guide constructs (SEQ ID NOs: 434, 655, 659, 663, and 668. FIG. 31B: Cleavage reactions are shown for MG119-28 and guide constructs (SEQ ID NOs: 686, 691, 703, and 704). For both effectors and guide panels, the intensity of the band corresponding to the nicked product does not significantly change relative to the WT sgRNA.

FIG. 32 depicts a phylogenetic tree of representative Cas 12 nucleases (600-1100 aa) highlighting novel MG families.

FIG. 33 depicts in vitro cleavage assay amplification products by automated gel electrophoresis. Example active MG191 proteins that successfully cleaved the PAM library yielded a band around 205 bp.

FIG. 34 depicts sequence logos of protospacer adjacent motifs (PAMs) obtained from NGS sequencing of the amplified cut site.

FIGS. 35A-35E depict an example purification and activity analysis of MG191-15. FIG. 35A: Protein expression induction and purification was monitored by gel electrophoresis. Expected protein MW was ~136 kDa. FIG. 35B: Concentrated protein was run over an S200i 10 300 SEC column, and peak fractions were collected and concentrated (shaded box). FIG. 35C: A normalized protein quantity of each protein prep was run on an SDS-PAGE protein gel for purification analysis via densitometry. FIG. 35D: Protein activity was assessed in an in vitro cleavage reaction, using linear DNA as substrate. Lanes: (1) substrate alone, (2) substrate+Apo (unguided) protein, (3) substrate+5× molar excess RNP, (4) substrate+25× molar excess RNP. FIG. 35E: automated electrophoresis gel showing nuclease activity for MG191-18, MG191-25, and MG191-29. Activity was detected by the presence of the amplified cleaved product bands around 250 bp (black arrow) when the pre-cRNA is present (+MA).

FIG. 36 depicts in-cell activity of MG119-28 with truncated sgRNAs. MG119-28 truncated guides were screened in K562 cells for activity at nine sites targeting hAAVS1. mRNA and guides were transfected in cells via nucleofection. Three days later, the edited sites were amplified for NGS and sequenced for INDEL analysis. All conditions were conducted in replicates of two.

FIG. 37 depicts dose titration of RNP to test best truncated scaffold potency. MG119-28 WT (filled circles) and sg1_8 truncated guide (triangles) RNPs were screened in K562 cells with three doses at nine sites targeting hAAVS1. RNPs were transfected in cells via nucleofection. Three days later, the edited sites were amplified for NGS and sequenced for INDEL analysis.

FIG. 38 depicts in cell activity with incremental truncations of MG119-28 sgRNA1. MG119-28 wild type guide was truncated at two stem loops in the repeat-anti-repeat region generating eight truncated designs. Guides carrying AAVS1 targeting spacers were transfected in K562s by nucleofection. Genomic DNA was analyzed for INDELs by NGS.

FIG. 39 depicts in vitro cleavage activity of modern and ancestral MG191 nucleases. Minimal arrays used in this experiment consist of repeat, spacer targeting the 8N PAM plasmid library, and repeat sequences. “A” stands for Apo condition where the minimal arrays are omitted. Each reaction is analyzed on a Tapestation to detect cleavage products between 200-300 bp. Ancestor candidates were tested with minimal arrays from closely related homologs. Modern candidates were tested with minimal array containing repeats in two orientations (forward and reverse). Cleavage was observed with MG191-51 and 191-51 ancestral candidates, as well as with modern MG191-1, MG191-2, MG191-5, and MG191-28 candidates.

FIGS. 40A-B depict sequence logos from NGS sequencing of in vitro cleavage activity products. FIG. 40A: Ancestral MG191-53 was active with crRNAs from active modern MG191 nucleases indicated on top of each sequence logo. The ancestral candidates were tested with minimal arrays, and active nucleases showed they are capable of processing the crepictRNAs from modern nucleases. MG191-53 prefers a PAM of TtR; the lower case letter means that there was a low signal. FIG. 40B: Modern MG191-5 nuclease is active with its corresponding crRNA. MG191-5 prefers a PAM of TRR.

FIG. 41 depicts gel electrophoretic analyses showing MG191 crRNAs are compatible with multiple MG191 candidates in vitro. Nucleases with similar known PAMs can be activated with the same crRNAs. Double strand cleavage is tested by incubating each nuclease with multiple minimal arrays consisting of repeat, spacer targeting the PAM plasmid library, and repeat sequences. Cleavage products 200-300 bp long are analyzed on 1.5% agarose gels and imaged. Apo conditions are tested without minimal arrays. PC=positive control nuclease and crRNA.

FIGS. 42A-C depict a SEC purification of Sumo-MG191-12 and activity analysis of SUMO-MG191-12 and SUMO-MG191-25. FIG. 42A: A SUMO-fused concentrated protein post IMAC is run over an S200i 10 300 SEC column, and peak fractions are collected, concentrated (shaded box; left) and run on an SDS-PAGE protein gel (right). FIG. 42B: Protein activity is assessed in an in vitro cleavage reaction, using 521 bp linear DNA as substrate. Lanes: (1) substrate alone, (2)-(5) substrate+1×, 5×, 25×, 40× molar excess of RNP over substrate FIG. 42C: Protein activity is assessed in an in vitro cleavage reaction, using 2,281 bp plasmid DNA as substrate. Lanes: (1) Substrate Alone, (2) Unguided: Protein at 40× molar excess over substrate with no SgRNA, (3)-(5) substrate+1×, 5×, 25×, 40× molar excess of RNP over substrate.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG122

SEQ ID NOs: 1-5 show the full-length amino acid sequences of MG122 nucleases.

### MG120

SEQ ID NOs: 6-14 show the full-length amino acid sequences of MG120 nucleases.

SEQ ID NOs: 333-335 and 355-357 show nucleotide sequences of MG120 tracrRNAs derived from the same loci as a MG120 Cas effector.

SEQ ID NOs: 374-375 and 389-390 show nucleotide sequences of MG120 minimal arrays.

### MG118

SEQ ID NO: 15 shows the full-length amino acid sequence of an MG118 nuclease.

SEQ ID NO: 376 shows a nucleotide sequence of an MG118 minimal array.

SEQ ID NO: 391 shows a nucleotide sequence of an MG118 minimal array.

SEQ ID NOs: 400-401 show nucleotide sequences of MG118 target CRISPR repeats.

SEQ ID NOs: 410-411 show nucleotide sequences of MG118 crRNAs.

### MG90

SEQ ID NOs: 16-29 show the full-length amino acid sequences of MG90 nucleases.

SEQ ID NOs: 346-347 and 368-369 show nucleotide sequences of MG90 tracrRNAs derived from the same loci as a MG90 Cas effector.

SEQ ID NOs: 383-384 and 398-399 show nucleotide sequences of MG90 minimal arrays.

SEQ ID NOs: 402-403 show nucleotide sequences of MG90 target CRISPR repeats.

SEQ ID NOs: 412-413 show nucleotide sequences of MG90 sgRNAs.

MG119

SEQ ID NOs: 30-150, 420-431, 476-624, and 629 show the full-length amino acid sequences of MG119 nucleases.

SEQ ID NOs: 326-332, 336-345, 348-354, and 358-367 show nucleotide sequences of MG119 tracrRNAs derived from the same loci as a MG119 Cas effector.

SEQ ID NOs: 370-373, 377-382, 385-388, and 392-397 show nucleotide sequences of MG119 minimal arrays.

SEQ ID NOs: 404-409 show nucleotide sequences of MG119 target CRISPR repeats.

SEQ ID NOs: 414-419, 432, 434, 436, 438, 440, 442, 444, 446, 448, 450, 452, 454, 456, 458, 460, 462, 464, 466, 468, 470, 472, 474, 647-766, and 1697-1731 show nucleotide sequences of MG119 sgRNAs.

SEQ ID NOs: 433, 435, 437, 439, 441, 443, 445, 447, 449, 451, 453, 455, 457, 459, 461, 463, 465, 467, 469, 471, 473, 475, and 646 show nucleotide sequences of MG119 PAMs.

### MG91B

SEQ ID NOs: 151-291 show the full-length amino acid sequences of MG91B nucleases.

### MG91C

SEQ ID NOs: 292-318 show the full-length amino acid sequences of MG91C nucleases.

### MG91A

SEQ ID NO: 319 shows the full-length amino acid sequence of an MG91A nuclease.

### MG126

SEQ ID NOs: 320-325 show the full-length amino acid sequences of MG126 nucleases.

### MG191

SEQ ID NOs: 1065-1090, 1114-1118, and 1142-1172 show the full-length amino acid sequences of MG191 nucleases.

SEQ ID NOs: 1091-1113 and 1119-1120 show nucleotide sequences of MG191 crRNAs.

SEQ ID NOs: 1732-1738 show nucleotide sequences of MG191 minimal arrays.

SEQ ID NOs: 1739-1745 and 1837-1841 show nucleotide sequences of MG191 PAMs.

SEQ ID NO: 1873 shows the nucleotide sequence of an active MG191 repeat sequence.

SEQ ID NO: 1874 shows the nucleotide sequence of an MG191 minimal array.

SEQ ID NO: 1875 shows the nucleotide sequence of an MG191 PAM sequence.

SEQ ID NO: 1876 shows the nucleotide sequence of an MG191 sgRNA

### MG185

SEQ ID NO: 1746 shows the full-length amino acid sequence of an MG185 nuclease.

### MG186

SEQ ID NOs: 1747-1748 show the full-length amino acid sequences of MG186 nucleases.

### MG187

SEQ ID NOs: 1749-1750 show the full-length amino acid sequences of MG187 nucleases.

### MG188

SEQ ID NOs: 1751-1752 show the full-length amino acid sequences of MG188 nucleases.

### Nuclear Localization Signals

SEQ ID NOs: 630-645 and 1904-1933 show the amino acid amino acid sequences of nuclear localization signals.

### TRAC Targeting

SEQ ID NOs: 767-798 show the nucleotide sequences of sgRNAs engineered to function with an MG119-2 nuclease in order to target TRAC.

SEQ ID NOs: 799-830 show the DNA sequences of TRAC target sites.

APOA1 Targeting with MG119-125

SEQ ID NOs: 831-904 show the nucleotide sequences of sgRNAs engineered to function with an MG119-125 nuclease in order to target APOA1.

SEQ ID NOs: 905-978 show the DNA sequences of APOA1 target sites.

APOA1 Targeting with MG119-129

SEQ ID NOs: 979-1021 show the nucleotide sequences of sgRNAs engineered to function with an MG119-129 nuclease in order to target APOA1.

SEQ ID NOs: 1022-1064 show the DNA sequences of APOA1 target sites.

Albumin (ALB) Targeting with MG119-2

SEQ ID NOs: 1121-1169 show the nucleotide sequences of sgRNAs engineered to function with an MG119-2 nuclease in order to target the albumin gene.

SEQ ID NOs: 1170-1218 show the DNA sequences of albumin gene target sites.

APOA1 Targeting with MG119-2

SEQ ID NOs: 1219-1237 show the nucleotide sequences of sgRNAs engineered to function with an MG119-2 nuclease in order to target APOA1.

SEQ ID NOs: 1238-1256 show the DNA sequences of APOA1 target sites.

AAVS1 Targeting with MG119-2

SEQ ID NOs: 1257-1324 show the nucleotide sequences of sgRNAs engineered to function with an MG119-2 nuclease in order to target AAVS1.

SEQ ID NOs: 1325-1392 show the DNA sequences of AAVS1 target sites.

Albumin (ALB) Targeting with MG119-28

SEQ ID NOs: 1393-1441, 1887, 1889, 1891, and 1892-1893 show the nucleotide sequences of sgRNAs engineered to function with an MG119-28 nuclease in order to target an albumin gene.

SEQ ID NOs: 1442-1490, 1888, 1890, and 1994 show the DNA sequences of albumin gene target sites.

APOA1 Targeting with MG119-28

SEQ ID NOs: 1491-1506 show the nucleotide sequences of sgRNAs engineered to function with an MG119-28 nuclease in order to target APOA1.

SEQ ID NOs: 1507-1522 show the DNA sequences of APOA1 target sites.

AAVS1 Targeting with MG119-28

SEQ ID NOs: 1523-1562, and 1753-1779 show the nucleotide sequences of sgRNAs engineered to function with an MG119-28 nuclease in order to target AAVS1.

SEQ ID NOs: 1563-1602 and 1780-1806 show the DNA sequences of AAVS1 target sites.

Albumin (ALB) Targeting with MG119-32

SEQ ID NOs: 1603-1632 show the nucleotide sequences of sgRNAs engineered to function with an MG119-32 nuclease in order to target albumin.

SEQ ID NOs: 1633-1662 show the DNA sequences of albumin target sites.

APOA1 Targeting with MG119-32

SEQ ID NOs: 1663-1669 show the nucleotide sequences of sgRNAs engineered to function with an MG119-32 nuclease in order to target APOA1.

SEQ ID NOs: 1670-1676 show the DNA sequences of APOA1 target sites.

AAVS1 Targeting with MG119-32

SEQ ID NOs: 1677-1686 show the nucleotide sequences of sgRNAs engineered to function with an MG119-32 nuclease in order to target AAVS1.

SEQ ID NOs: 1687-1696 show the DNA sequences of AAVS1 target sites.

### U67 Spacer DNA

SEQ ID NOs: 1877 and 1891 show the nucleotide sequences of U67 Spacer DNAs U40 Spacer DNA

SEQ ID NO: 1878 shows the nucleotide sequence of a U40 Spacer DNA.

### Oligonucleotide Primers for Nucleic Acid Synthesis

SEQ ID Nos; 1895, 1896, 1897, 1898, and 1899 show the nucleotide sequences of 611F_HE, 869R_HE, 680_HE Taqman Probe, 611 NGS, and 927 R NGS DNA primers for nucleic acid synthesis.

### SUMO Protein Sequences

SEQ ID NO: 1900 shows the amino acid sequence of a SUMO protein.

### Precission Protease Sequences

SEQ ID NO: 1902 shows the amino acid sequence of a Precission protease site.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The practice of some methods disclosed herein employ, unless otherwise indicated, techniques of immunology, biochemistry, chemistry, molecular biology, microbiology, cell biology, genomics, and recombinant DNA.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink Deoxy Nucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

The terms “transfection” or “transfected” refer to introduction of a nucleic acid into a cell by non-viral or viral-based methods. The nucleic acid molecules may be gene sequences encoding complete proteins or functional portions thereof.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. Eukaryotic basal promoters typically, though not necessarily, contain a TATA-box and/or a CAAT box.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, the term expression includes splicing of the mRNA in a eukaryotic cell.

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, “expression cassette” and “nucleic acid cassette” are used interchangeably to refer to a component of a vector comprising a combination of nucleic acid sequences or elements (e.g., therapeutic gene, promoter, and a terminator) that are expressed together or are operably linked for expression. The terms encompass an expression cassette including a combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence,” or a “spacer.” A nucleic acid-targeting segment can include a sub-segment referred to as a “protein binding segment” or “protein binding sequence” or “Cas protein binding segment.”

As used herein, the term “Cas12a” refers to a family of Cas endonucleases that are class 2, Type V-A Cas endonucleases and that (a) use a relatively small guide RNA (about 42-44 nucleotides) that is processed by the nuclease itself following transcription from the CRISPR array, and (b) cleave DNA to leave staggered cut sites. Further features of this family of enzymes can be found, e.g. in Zetsche B, Heidenreich M, Mohanraju P, et al. Nat Biotechnol 2017; 35:31-34, and Zetsche B, Gootenberg J S, Abudayyeh O O, et al. Cell 2015; 163:759-771.

The term “tracrRNA” or “tracr sequence” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

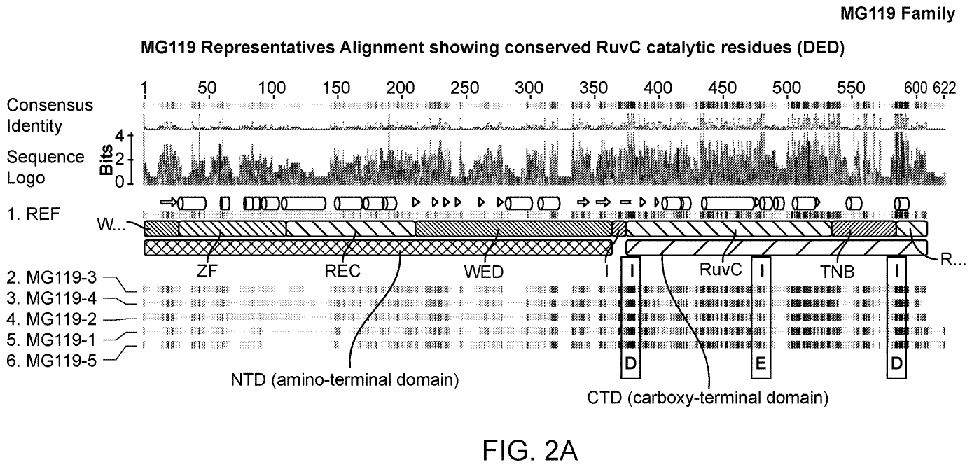

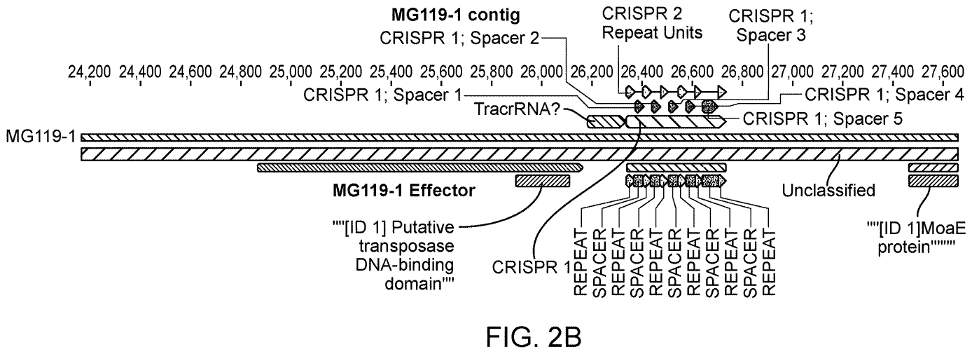

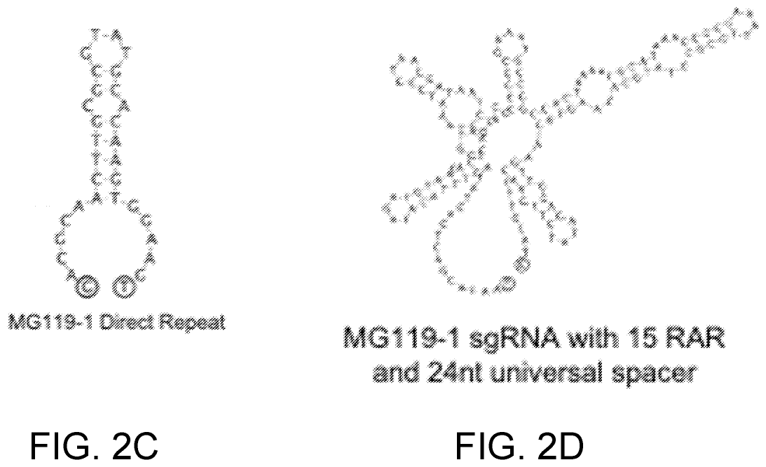

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the endonuclease protein sequences described herein (e.g. MG90, MG91A, MG91B, MG91C, MG118, MG119, MG120, MG122, MG126, or MG191 family endonucleases described herein, or any other family nuclease described herein). In some embodiments, such conservatively substituted variants are functional variants. Such functional variants can encompass sequences with substitutions such that the activity of one or more critical active site residues or guide RNA binding residues of the endonuclease are not disrupted. In some embodiments, a functional variant of any of the proteins described herein lacks substitution of at least one of the conserved or functional residues called out in FIG. 2A, 3A, 4A, 5A, or 6A. In some embodiments, a functional variant of any of the proteins described herein lacks substitution of all of the conserved or functional residues called out in FIG. 2A, 3A, 4A, 5A, or 6A.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIG. 2A, 3A, 4A, 5A, or 6A.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

### Overview

The discovery of new Cas enzymes with unique functionality and structure offers the potential to further gene editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches containing large numbers of microbial species may offer the potential to drastically increase the number of new CRISPR/Cas systems characterized and speed the discovery of new oligonucleotide editing functionalities. A recent example of the fruitfulness of such an approach is demonstrated by the 2016 discovery of CasX/CasY CRISPR systems from metagenomic analysis of natural microbial communities.

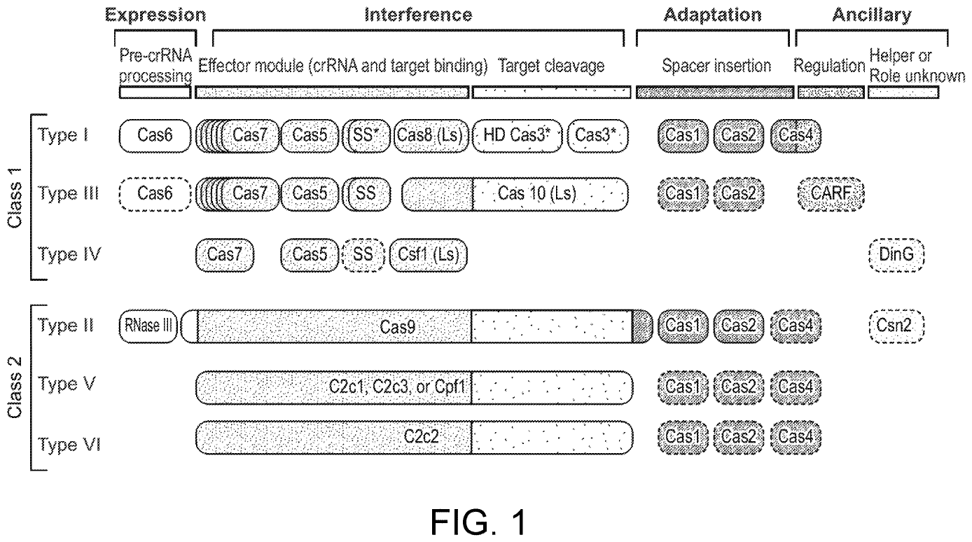

CRISPR/Cas systems are RNA-directed nuclease complexes that function as an adaptive immune system in microbes. In their natural context, CRISPR/Cas systems occur in CRISPR (clustered regularly interspaced short palindromic repeats) operons or loci, which generally are made up of two parts: (i) an array of short repetitive sequences (30-40 bp) separated by short spacer sequences, which encode the RNA-based targeting element; and (ii) ORFs encoding the Cas nuclease. Efficient nuclease targeting of a particular target nucleic acid sequence generally requires both (i) complementary hybridization between the first 6-8 nucleic acids of the target nucleic acid and a crRNA guide; and (ii) presence of a protospacer-adjacent motif (PAM) sequence within a certain vicinity of the target nucleic acid sequence depending on the specific Cas nuclease (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class 1 CRISPR-Cas systems have large, multi-subunit effector complexes, and include Types I, III, and IV Cas nucleases. Class 2 CRISPR-Cas systems generally have single-polypeptide multidomain nuclease effectors, and include Types II, V and VI Cas nucleases.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g. Cas9) and the repeat sequence to form a precursor dsRNA structure, which is cleaved by endogenous RNAse III to generate a mature effector enzyme loaded with both tracrRNA and crRNA. Cas II nucleases are identified as DNA nucleases. Type 2 effectors generally exhibit a structure comprising a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g. Cas12) structure similar to that of Type II effectors, comprising a RuvC-like domain. Similar to Type II, most (but not all) Type V CRISPR systems use a tracrRNA to process pre-crRNAs into mature crRNAs. However, unlike Type II systems which requires RNAse III to cleave the pre-crRNA into multiple crRNAs. Type V systems are capable of using the effector nuclease itself to cleave pre-crRNAs. Like Type-II CRISPR-Cas systems, Type V CRISPR-Cas systems are again identified as DNA nucleases. Unlike Type II CRISPR-Cas systems, some Type V enzymes (e.g., Cas 12a) appear to have a robust single-stranded nonspecific deoxyribonuclease activity that is activated by the first crRNA directed cleavage of a double-stranded target sequence.

CRISPR-Cas systems have emerged in recent years as the gene editing technology of choice due to their targetability and ease of use. The most commonly used systems are the Class 2 Type II SpCas9 and the Class 2 Type V-A Cas12a (previously Cpf1). The Type V-A systems in particular are becoming more widely used since their reported specificity in cells is higher than other nucleases, with fewer or no off-target effects. The V-A systems are also advantageous in that the guide RNA is small (42-44 nucleotides compared with approximately 100 nt for SpCas9) and is processed by the nuclease itself following transcription from the CRISPR array, simplifying multiplexed applications with multiple gene edits. Furthermore, the V-A systems have staggered cut sites, which may facilitate directed repair pathways, such as microhomology-dependent targeted integration (MITI).

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. Recent exploration of orthologs has revealed proteins with less restrictive PAM sequences that are also active in mammalian cell culture, for example YTV, YYN or TTN. However, these enzymes do not fully encompass Type V biodiversity and targetability, and may not represent all possible activities and PAM sequence requirements. Described herein, in certain embodiments, are improved Type V nucleases that are highly targetable, compact, and precise for use in systems and methods for gene editing.

### MG Enzymes

Described herein, in certain embodiments, are engineered nuclease systems comprising an endonuclease and an engineered guide polynucleotide. In some embodiments, the endonuclease is a Class 2, Type V endonuclease. In some embodiments, the endonuclease is a double-strand nuclease. In some embodiments, the endonuclease is catalytically dead. In some embodiments, the endonuclease is modified. In some embodiments, the endonuclease is modified resulting in an endonuclease with nickase activity. In some embodiments, the modified endonuclease is a site-directed nickase.

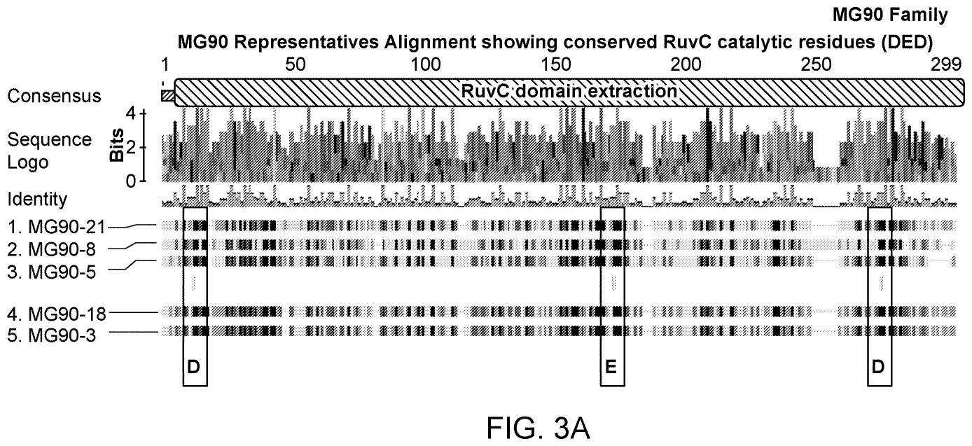

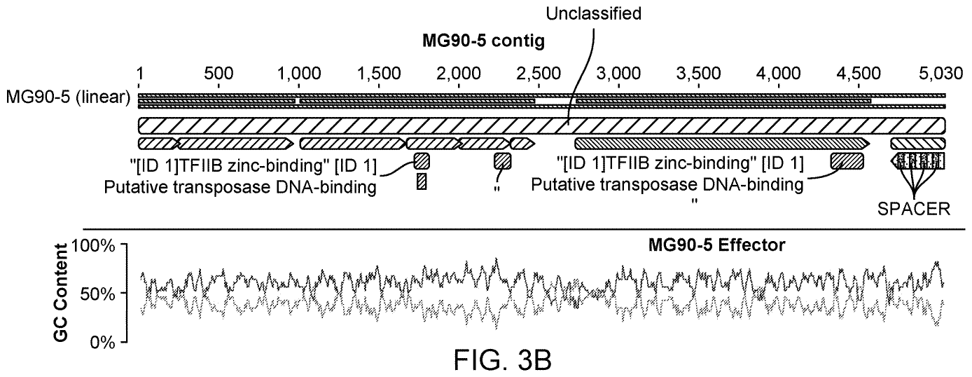

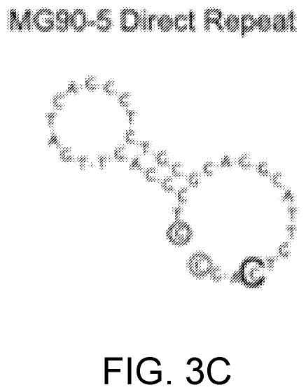

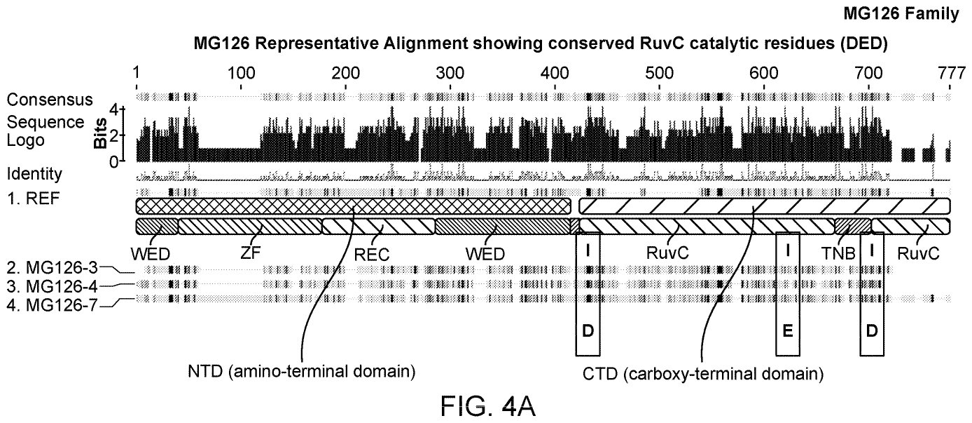

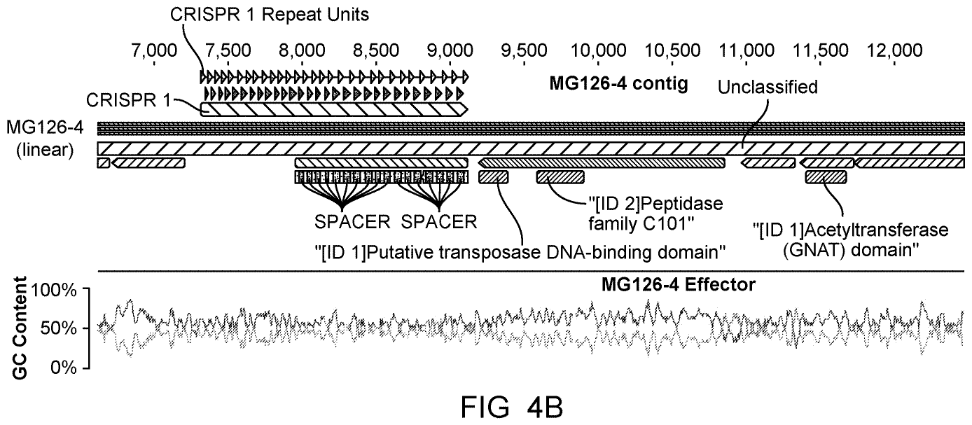

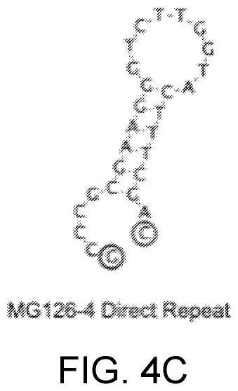

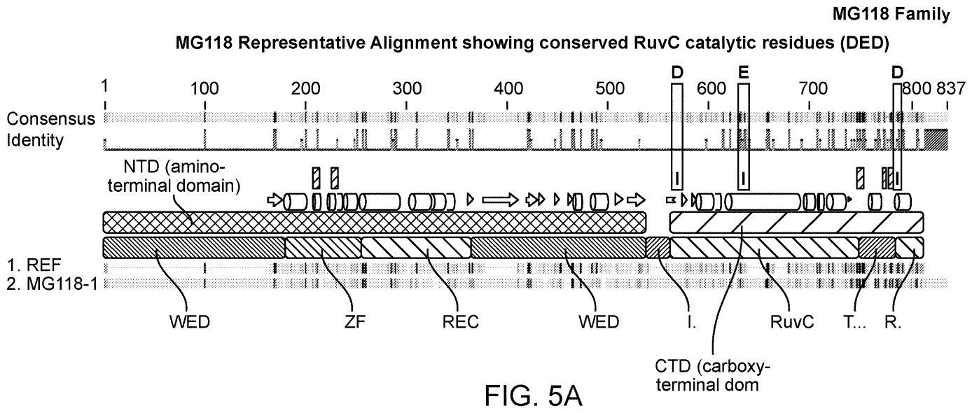

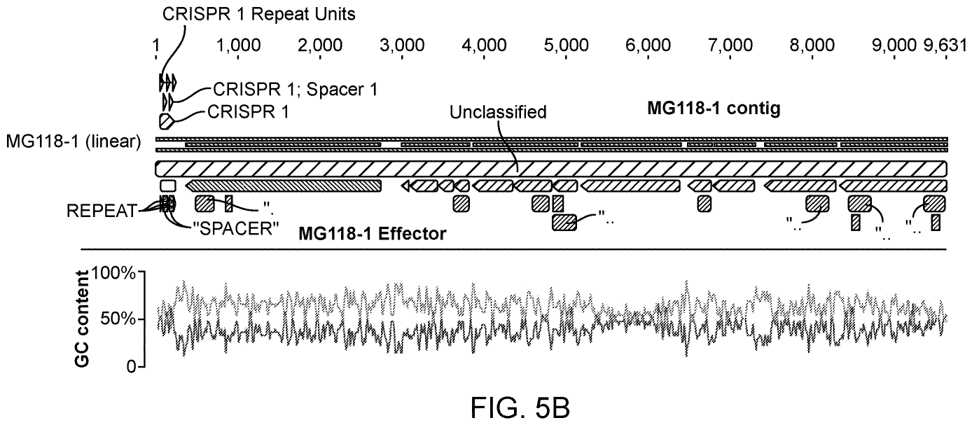

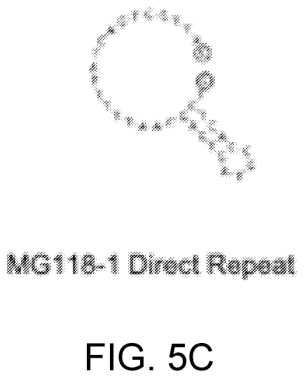

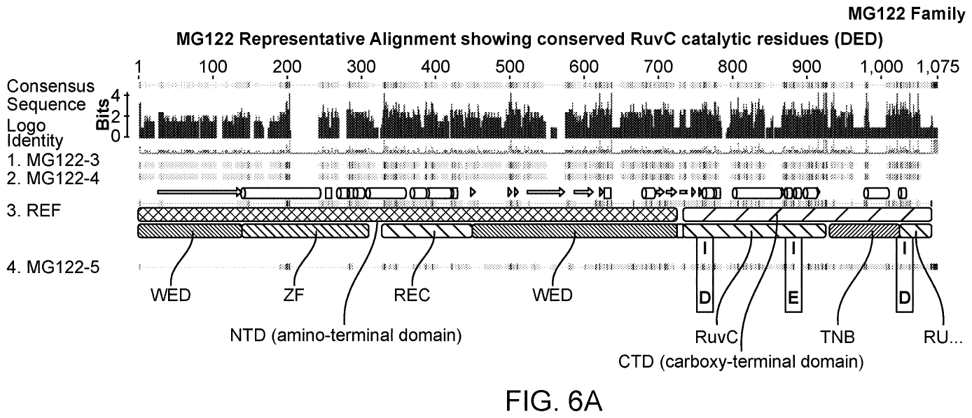

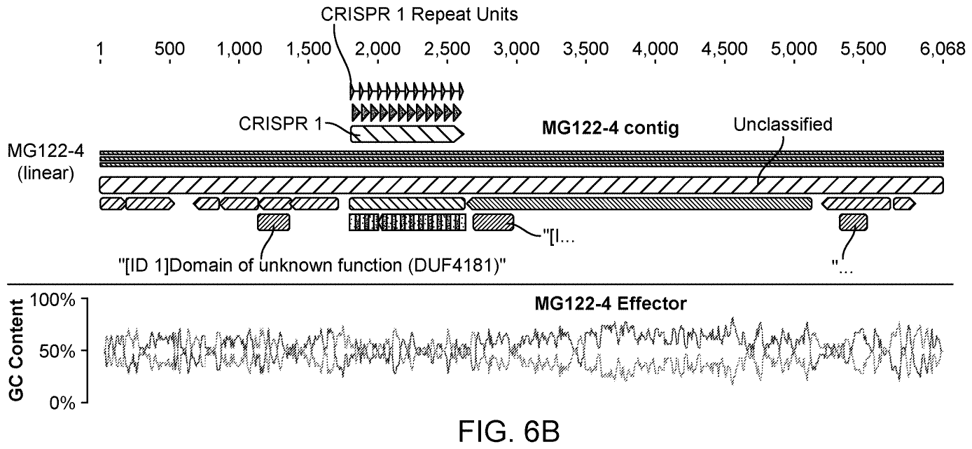

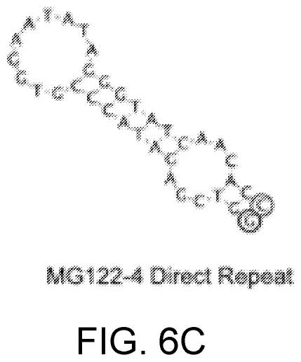

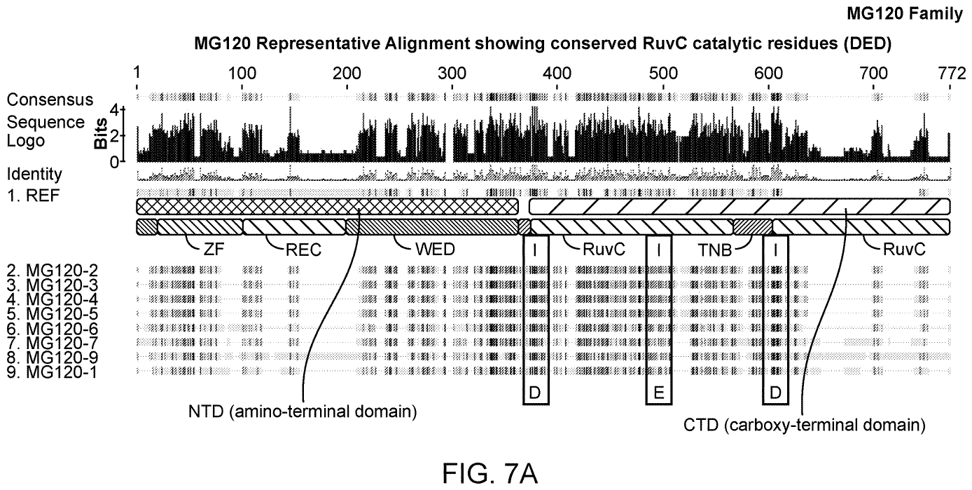

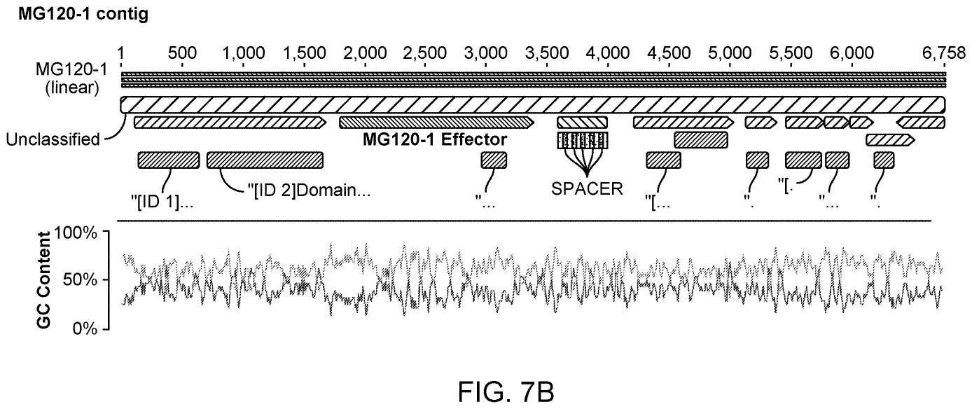

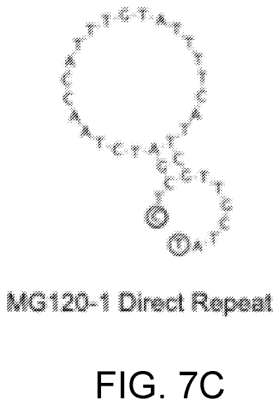

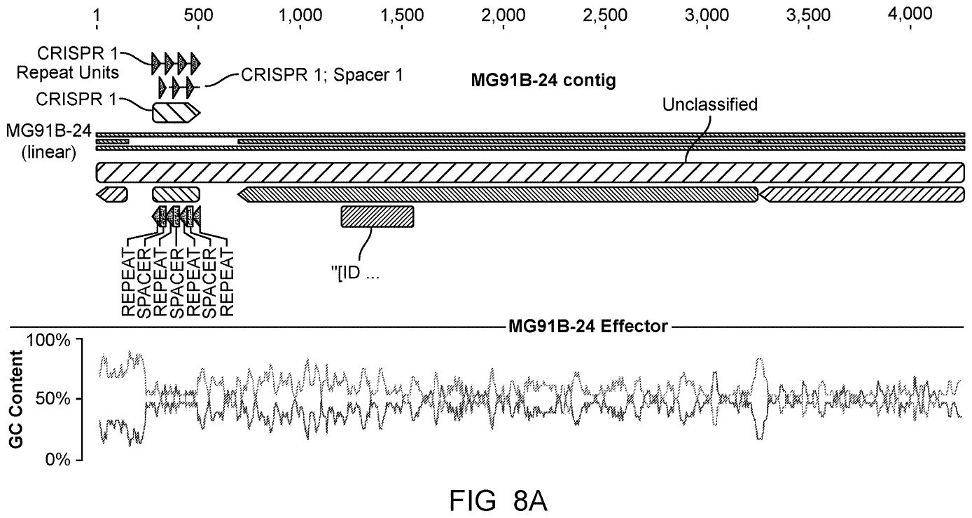

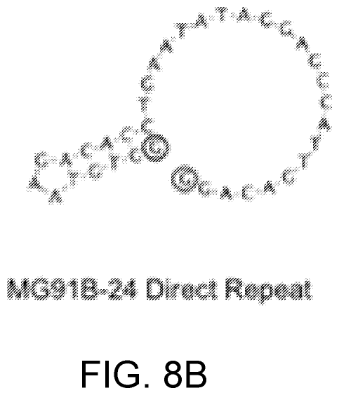

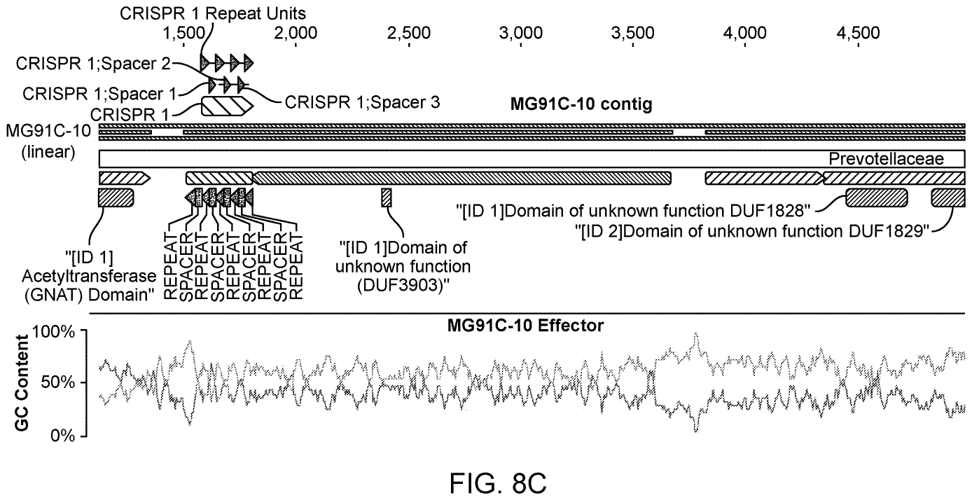

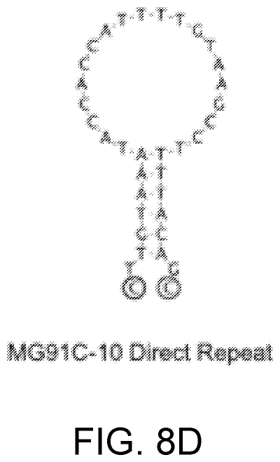

In some embodiments, the endonuclease is an MG90 endonuclease (see FIGS. 3A-3C). In some embodiments, the endonuclease is an MG90A endonuclease. In some embodiments, the endonuclease is an MG90B endonuclease. In some embodiments, the endonuclease is an MG90C endonuclease. In some embodiments, the endonuclease is an MG91 endonuclease (see FIGS. 8A-8B). In some embodiments, the endonuclease is an MG118 endonuclease (see FIGS. 5A-5C). In some embodiments, the endonuclease is an MG119 endonuclease (see FIGS. 2A-2D). In some embodiments, the endonuclease is an MG120 endonuclease (see FIGS. 7A-7C). In some embodiments, the endonuclease is an MG122 endonuclease (see FIGS. 6A-6C). In some embodiments, the endonuclease is an MG126 endonuclease (see FIGS. 4A-4C). In some embodiments, the endonuclease is an MG191 endonuclease.

In some embodiments, the nucleases are less than about 1000 amino acids in length. In some embodiments, the nucleases are less than about 900 amino acids in length. In some embodiments, the nucleases are less than about 850 amino acids in length. In some embodiments, the nucleases are less than about 800 amino acids in length. In some embodiments, the nucleases are less than about 750 amino acids in length. In some embodiments, the nucleases are less than about 700 amino acids in length. In some embodiments, the nucleases are less than about 650 amino acids in length. In some embodiments, the nucleases are less than about 600 amino acids in length. In some embodiments, the nucleases are less than about 550 amino acids in length. In some embodiments, the nucleases are less than about 500 amino acids in length.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1-5. In some embodiments, the endonuclease comprises a sequence having at least about 70% identity to any one of SEQ ID NOs: 1-5. In some embodiments, the endonuclease comprises a sequence having at least about 75% identity to any one of SEQ ID NOs: 1-5. In some embodiments, the endonuclease comprises a sequence having at least about 80% identity to any one of SEQ ID NOs: 1-5. In some embodiments, the endonuclease comprises a sequence having at least about 85% identity to any one of SEQ ID NOs: 1-5. In some embodiments, the endonuclease comprises a sequence having at least about 90% identity to any one of SEQ ID NOs: 1-5. In some embodiments, the endonuclease comprises a sequence having at least about 95% identity to any one of SEQ ID NOs: 1-5. In some embodiments, the endonuclease comprises a sequence having at least about 96% identity to any one of SEQ ID NOs: 1-5. In some embodiments, the endonuclease comprises a sequence having at least about 97% identity to any one of SEQ ID NOs: 1-5. In some embodiments, the endonuclease comprises a sequence having at least about 98% identity to any one of SEQ ID NOs: 1-5. In some embodiments, the endonuclease comprises a sequence having at least about 99% identity to any one of SEQ ID NOs: 1-5. In some embodiments, the endonuclease comprises a sequence having 100% identity to any one of SEQ ID NOs: 1-5.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 6-14. In some embodiments, the endonuclease comprises a sequence having at least about 70% identity to any one of SEQ ID NOs: 6-14. In some embodiments, the endonuclease comprises a sequence having at least about 75% identity to any one of SEQ ID NOs: 6-14. In some embodiments, the endonuclease comprises a sequence having at least about 80% identity to any one of SEQ ID NOs: 6-14. In some embodiments, the endonuclease comprises a sequence having at least about 85% identity to any one of SEQ ID NOs: 6-14. In some embodiments, the endonuclease comprises a sequence having at least about 90% identity to any one of SEQ ID NOs: 6-14. In some embodiments, the endonuclease comprises a sequence having at least about 95% identity to any one of SEQ ID NOs: 6-14. In some embodiments, the endonuclease comprises a sequence having at least about 96% identity to any one of SEQ ID NOs: 6-14. In some embodiments, the endonuclease comprises a sequence having at least about 97% identity to any one of SEQ ID NOs: 6-14. In some embodiments, the endonuclease comprises a sequence having at least about 98% identity to any one of SEQ ID NOs: 6-14. In some embodiments, the endonuclease comprises a sequence having at least about 99% identity to any one of SEQ ID NOs: 6-14. In some embodiments, the endonuclease comprises a sequence having 100% identity to any one of SEQ ID NOs: 6-14.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to SEQ ID NO: 15. In some embodiments, the endonuclease comprises a sequence having at least about 70% identity to SEQ ID NO: 15. In some embodiments, the endonuclease comprises a sequence having at least about 75% identity to SEQ ID NO: 15. In some embodiments, the endonuclease comprises a sequence having at least about 80% identity to SEQ ID NO: 15. In some embodiments, the endonuclease comprises a sequence having at least about 85% identity to SEQ ID NO: 15. In some embodiments, the endonuclease comprises a sequence having at least about 90% identity to SEQ ID NO: 15. In some embodiments, the endonuclease comprises a sequence having at least about 95% identity to SEQ ID NO: 15. In some embodiments, the endonuclease comprises a sequence having at least about 96% identity to SEQ ID NO: 15. In some embodiments, the endonuclease comprises a sequence having at least about 97% identity to SEQ ID NO: 15. In some embodiments, the endonuclease comprises a sequence having at least about 98% identity to SEQ ID NO: 15. In some embodiments, the endonuclease comprises a sequence having at least about 99% identity to SEQ ID NO: 15. In some embodiments, the endonuclease comprises a sequence having 100% identity to SEQ ID NO: 15.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 16-29. In some embodiments, the endonuclease comprises a sequence having at least about 70% identity to any one of SEQ ID NOs: 16-29. In some embodiments, the endonuclease comprises a sequence having at least about 75% identity to any one of SEQ ID NOs: 16-29. In some embodiments, the endonuclease comprises a sequence having at least about 80% identity to any one of SEQ ID NOs: 16-29. In some embodiments, the endonuclease comprises a sequence having at least about 85% identity to any one of SEQ ID NOs: 16-29. In some embodiments, the endonuclease comprises a sequence having at least about 90% identity to any one of SEQ ID NOs: 16-29. In some embodiments, the endonuclease comprises a sequence having at least about 95% identity to any one of SEQ ID NOs: 16-29. In some embodiments, the endonuclease comprises a sequence having at least about 96% identity to any one of SEQ ID NOs: 16-29. In some embodiments, the endonuclease comprises a sequence having at least about 97% identity to any one of SEQ ID NOs: 16-29. In some embodiments, the endonuclease comprises a sequence having at least about 98% identity to any one of SEQ ID NOs: 16-29. In some embodiments, the endonuclease comprises a sequence having at least about 99% identity to any one of SEQ ID NOs: 16-29. In some embodiments, the endonuclease comprises a sequence having 100% identity to any one of SEQ ID NOs: 16-29.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 30-150, 420-431, 476-624, and 629. In some embodiments, the endonuclease comprises a sequence having at least about 70% identity to any one of SEQ ID NOs: 30-150, 420-431, 476-624, and 629. In some embodiments, the endonuclease comprises a sequence having at least about 75% identity to any one of SEQ ID NOs: 30-150, 420-431, 476-624, and 629. In some embodiments, the endonuclease comprises a sequence having at least about 80% identity to any one of SEQ ID NOs: 30-150, 420-431, 476-624, and 629. In some embodiments, the endonuclease comprises a sequence having at least about 85% identity to any one of SEQ ID NOs: 30-150, 420-431, 476-624, and 629. In some embodiments, the endonuclease comprises a sequence having at least about 90% identity to any one of SEQ ID NOs: 30-150, 420-431, 476-624, and 629. In some embodiments, the endonuclease comprises a sequence having at least about 95% identity to any one of SEQ ID NOs: 30-150, 420-431, 476-624, and 629. In some embodiments, the endonuclease comprises a sequence having at least about 96% identity to any one of SEQ ID NOs: 30-150, 420-431, 476-624, and 629. In some embodiments, the endonuclease comprises a sequence having at least about 97% identity to any one of SEQ ID NOs: 30-150, 420-431, 476-624, and 629. In some embodiments, the endonuclease comprises a sequence having at least about 98% identity to any one of SEQ ID NOs: 30-150, 420-431, 476-624, and 629. In some embodiments, the endonuclease comprises a sequence having at least about 99% identity to any one of SEQ ID NOs: 30-150, 420-431, 476-624, and 629. In some embodiments, the endonuclease comprises a sequence having 100% identity to any one of SEQ ID NOs: 30-150, 420-431, 476-624, and 629.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NO: 151-291. In some embodiments, the endonuclease comprises a sequence having at least about 70% identity to any one of SEQ ID NO: 151-291. In some embodiments, the endonuclease comprises a sequence having at least about 75% identity to any one of SEQ ID NO: 151-291. In some embodiments, the endonuclease comprises a sequence having at least about 80% identity to any one of SEQ ID NO: 151-291. In some embodiments, the endonuclease comprises a sequence having at least about 85% identity to any one of SEQ ID NO: 151-291. In some embodiments, the endonuclease comprises a sequence having at least about 90% identity to any one of SEQ ID NO: 151-291. In some embodiments, the endonuclease comprises a sequence having at least about 95% identity to any one of SEQ ID NO: 151-291. In some embodiments, the endonuclease comprises a sequence having at least about 96% identity to any one of SEQ ID NO: 151-291. In some embodiments, the endonuclease comprises a sequence having at least about 97% identity to any one of SEQ ID NO: 151-291. In some embodiments, the endonuclease comprises a sequence having at least about 98% identity to any one of SEQ ID NO: 151-291. In some embodiments, the endonuclease comprises a sequence having at least about 99% identity to any one of SEQ ID NO: 151-291. In some embodiments, the endonuclease comprises a sequence having 100% identity to any one of SEQ ID NO: 151-291.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 292-318. In some embodiments, the endonuclease comprises a sequence having at least about 70% identity to any one of SEQ ID NOs: 292-318. In some embodiments, the endonuclease comprises a sequence having at least about 75% identity to any one of SEQ ID NOs: 292-318. In some embodiments, the endonuclease comprises a sequence having at least about 80% identity to any one of SEQ ID NOs: 292-318. In some embodiments, the endonuclease comprises a sequence having at least about 85% identity to any one of SEQ ID NOs: 292-318. In some embodiments, the endonuclease comprises a sequence having at least about 90% identity to any one of SEQ ID NOs: 292-318. In some embodiments, the endonuclease comprises a sequence having at least about 95% identity to any one of SEQ ID NOs: 292-318. In some embodiments, the endonuclease comprises a sequence having at least about 96% identity to any one of SEQ ID NOs: 292-318. In some embodiments, the endonuclease comprises a sequence having at least about 97% identity to any one of SEQ ID NOs: 292-318. In some embodiments, the endonuclease comprises a sequence having at least about 98% identity to any one of SEQ ID NOs: 292-318. In some embodiments, the endonuclease comprises a sequence having at least about 99% identity to any one of SEQ ID NOs: 292-318. In some embodiments, the endonuclease comprises a sequence having 100% identity to any one of SEQ ID NOs: 292-318.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to SEQ ID NO: 319. In some embodiments, the endonuclease comprises a sequence having at least about 70% identity to SEQ ID NO: 319. In some embodiments, the endonuclease comprises a sequence having at least about 75% identity to SEQ ID NO: 319. In some embodiments, the endonuclease comprises a sequence having at least about 80% identity to SEQ ID NO: 319. In some embodiments, the endonuclease comprises a sequence having at least about 85% identity to SEQ ID NO: 319. In some embodiments, the endonuclease comprises a sequence having at least about 90% identity to SEQ ID NO: 319. In some embodiments, the endonuclease comprises a sequence having at least about 95% identity to SEQ ID NO: 319. In some embodiments, the endonuclease comprises a sequence having at least about 96% identity to SEQ ID NO: 319. In some embodiments, the endonuclease comprises a sequence having at least about 97% identity to SEQ ID NO: 319. In some embodiments, the endonuclease comprises a sequence having at least about 98% identity to SEQ ID NO: 319. In some embodiments, the endonuclease comprises a sequence having at least about 99% identity to SEQ ID NO: 319. In some embodiments, the endonuclease comprises a sequence having 100% identity to SEQ ID NO: 319.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 320-325. In some embodiments, the endonuclease comprises a sequence having at least about 70% identity to any one of SEQ ID NOs: 320-325. In some embodiments, the endonuclease comprises a sequence having at least about 75% identity to any one of SEQ ID NOs: 320-325. In some embodiments, the endonuclease comprises a sequence having at least about 80% identity to any one of SEQ ID NOs: 320-325. In some embodiments, the endonuclease comprises a sequence having at least about 85% identity to any one of SEQ ID NOs: 320-325. In some embodiments, the endonuclease comprises a sequence having at least about 90% identity to any one of SEQ ID NOs: 320-325. In some embodiments, the endonuclease comprises a sequence having at least about 95% identity to any one of SEQ ID NOs: 320-325. In some embodiments, the endonuclease comprises a sequence having at least about 96% identity to any one of SEQ ID NOs: 320-325. In some embodiments, the endonuclease comprises a sequence having at least about 97% identity to any one of SEQ ID NOs: 320-325. In some embodiments, the endonuclease comprises a sequence having at least about 98% identity to any one of SEQ ID NOs: 320-325. In some embodiments, the endonuclease comprises a sequence having at least about 99% identity to any one of SEQ ID NOs: 320-325. In some embodiments, the endonuclease comprises a sequence having 100% identity to any one of SEQ ID NOs: 320-325.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1065-1090 and 1114-1118. In some embodiments, the endonuclease comprises a sequence having at least about 70% identity to any one of SEQ ID NOs: 1065-1090 and 1114-1118. In some embodiments, the endonuclease comprises a sequence having at least about 75% identity to any one of SEQ ID NOs: 1065-1090 and 1114-1118. In some embodiments, the endonuclease comprises a sequence having at least about 80% identity to any one of SEQ ID NOs: 1065-1090 and 1114-1118. In some embodiments, the endonuclease comprises a sequence having at least about 85% identity to any one of SEQ ID NOs: 1065-1090 and 1114-1118. In some embodiments, the endonuclease comprises a sequence having at least about 90% identity to any one of SEQ ID NOs: 1065-1090 and 1114-1118. In some embodiments, the endonuclease comprises a sequence having at least about 95% identity to any one of SEQ ID NOs: 1065-1090 and 1114-1118. In some embodiments, the endonuclease comprises a sequence having at least about 96% identity to any one of SEQ ID NOs: 1065-1090 and 1114-1118. In some embodiments, the endonuclease comprises a sequence having at least about 97% identity to any one of SEQ ID NOs: 1065-1090 and 1114-1118. In some embodiments, the endonuclease comprises a sequence having at least about 98% identity to any one of SEQ ID NOs: 1065-1090 and 1114-1118. In some embodiments, the endonuclease comprises a sequence having at least about 99% identity to any one of SEQ ID NOs: 1065-1090 and 1114-1118. In some embodiments, the endonuclease comprises a sequence having 100% identity to any one of SEQ ID NOs: 1065-1090 and 1114-1118.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to SEQ ID NO: 1746. In some embodiments, the endonuclease comprises a sequence having at least about 70% identity to SEQ ID NO: 1746. In some embodiments, the endonuclease comprises a sequence having at least about 75% identity to SEQ ID NO: 1746. In some embodiments, the endonuclease comprises a sequence having at least about 80% identity to SEQ ID NO: 1746. In some embodiments, the endonuclease comprises a sequence having at least about 85% identity to SEQ ID NO: 1746. In some embodiments, the endonuclease comprises a sequence having at least about 90% identity to SEQ ID NO: 1746. In some embodiments, the endonuclease comprises a sequence having at least about 95% identity to SEQ ID NO: 1746. In some embodiments, the endonuclease comprises a sequence having at least about 96% identity to SEQ ID NO: 1746. In some embodiments, the endonuclease comprises a sequence having at least about 97% identity to SEQ ID NO: 1746. In some embodiments, the endonuclease comprises a sequence having at least about 98% identity to SEQ ID NO: 1746. In some embodiments, the endonuclease comprises a sequence having at least about 99% identity to SEQ ID NO: 1746. In some embodiments, the endonuclease comprises a sequence having 100% identity to SEQ ID NO: 1746.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1747-1748. In some embodiments, the endonuclease comprises a sequence having at least about 70% identity to any one of SEQ ID NOs: 1747-1748. In some embodiments, the endonuclease comprises a sequence having at least about 75% identity to any one of SEQ ID NOs: 1747-1748. In some embodiments, the endonuclease comprises a sequence having at least about 80% identity to any one of SEQ ID NOs: 1747-1748. In some embodiments, the endonuclease comprises a sequence having at least about 85% identity to any one of SEQ ID NOs: 1747-1748. In some embodiments, the endonuclease comprises a sequence having at least about 90% identity to any one of SEQ ID NOs: 1747-1748. In some embodiments, the endonuclease comprises a sequence having at least about 95% identity to any one of SEQ ID NOs: 1747-1748. In some embodiments, the endonuclease comprises a sequence having at least about 96% identity to any one of SEQ ID NOs: 1747-1748. In some embodiments, the endonuclease comprises a sequence having at least about 97% identity to any one of SEQ ID NOs: 1747-1748. In some embodiments, the endonuclease comprises a sequence having at least about 98% identity to any one of SEQ ID NOs: 1747-1748. In some embodiments, the endonuclease comprises a sequence having at least about 99% identity to any one of SEQ ID NOs: 1747-1748. In some embodiments, the endonuclease comprises a sequence having 100% identity to any one of SEQ ID NOs: 1747-1748.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1749-1750. In some embodiments, the endonuclease comprises a sequence having at least about 70% identity to any one of SEQ ID NOs: 1749-1750. In some embodiments, the endonuclease comprises a sequence having at least about 75% identity to any one of SEQ ID NOs: 1749-1750. In some embodiments, the endonuclease comprises a sequence having at least about 80% identity to any one of SEQ ID NOs: 1749-1750. In some embodiments, the endonuclease comprises a sequence having at least about 85% identity to any one of SEQ ID NOs: 1749-1750. In some embodiments, the endonuclease comprises a sequence having at least about 90% identity to any one of SEQ ID NOs: 1749-1750. In some embodiments, the endonuclease comprises a sequence having at least about 95% identity to any one of SEQ ID NOs: 1749-1750. In some embodiments, the endonuclease comprises a sequence having at least about 96% identity to any one of SEQ ID NOs: 1749-1750. In some embodiments, the endonuclease comprises a sequence having at least about 97% identity to any one of SEQ ID NOs: 1749-1750. In some embodiments, the endonuclease comprises a sequence having at least about 98% identity to any one of SEQ ID NOs: 1749-1750. In some embodiments, the endonuclease comprises a sequence having at least about 99% identity to any one of SEQ ID NOs: 1749-1750. In some embodiments, the endonuclease comprises a sequence having 100% identity to any one of SEQ ID NOs: 1749-1750.

In some embodiments, the endonuclease comprises a sequence having at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99% identity to any one of SEQ ID NOs: 1751-1752. In some embodiments, the endonuclease comprises a sequence having at least about 70% identity to any one of SEQ ID NOs: 1751-1752. In some embodiments, the endonuclease comprises a sequence having at least about 75% identity to any one of SEQ ID NOs: 1751-1752. In some embodiments, the endonuclease comprises a sequence having at least about 80% identity to any one of SEQ ID NOs: 1751-1752. In some embodiments, the endonuclease comprises a sequence having at least about 85% identity to any one of SEQ ID NOs: 1751-1752. In some embodiments, the endonuclease comprises a sequence having at least about 90% identity to any one of SEQ ID NOs: 1751-1752. In some embodiments, the endonuclease comprises a sequence having at least about 95% identity to any one of SEQ ID NOs: 1751-1752. In some embodiments, the endonuclease comprises a sequence having at least about 96% identity to any one of SEQ ID NOs: 1751-1752. In some embodiments, the endonuclease comprises a sequence having at least about 97% identity to any one of SEQ ID NOs: 1751-1752. In some embodiments, the endonuclease comprises a sequence having at least about 98% identity to any one of SEQ ID NOs: 1751-1752. In some embodiments, the endonuclease comprises a sequence having at least about 99% identity to any one of SEQ ID NOs: 1751-1752. In some embodiments, the endonuclease comprises a sequence having 100% identity to any one of SEQ ID NOs: 1751-1752.

In some embodiments, the nucleases comprise RuvC catalytic residues. In some embodiments, the nucleases do not require tracrRNA. In some embodiments, the nucleases comprise RuvC catalytic residues and do not require tracrRNA.

In some embodiments, the nucleases comprise a PAM interacting domain. In some embodiments, the PAM comprises a sequence of any one of SEQ ID NOs: 433, 435, 437, 439, 441, 443, 445, 447, 449, 451, 453, 455, 457, 459, 461, 463, 465, 467, 469, 471, 473, 475, and 646.

In some embodiments, the engineered nuclease system is discovered through metagenomic sequencing. In some embodiments, the metagenomic sequencing is conducted on samples collected from various environments. In some embodiments, the environment is a human microbiome, an animal microbiome, an environment with high temperatures, an environment with low temperatures, or sediment.

In some embodiments, the endonuclease comprises a nuclear localization sequence (NLS). In some embodiments, the NLS is at an N-terminus of the endonuclease. In some embodiments, the NLS is at a C-terminus of the endonuclease. In some embodiments, the NLS is at an N-terminus and a C-terminus of the endonuclease.

In some embodiments, the NLS comprises a sequence of any one of SEQ ID NOs: 630-645 and 1904-1933, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 630-645 and 1904-1933. In some cases, the NLS comprises a sequence having at least about 80% identity to SEQ ID NOs: 630-645 and 1904-1933. In some cases, the NLS comprises a sequence having at least about 85% identity to SEQ ID NOs: 630-645 and 1904-1933. In some cases, the NLS comprises a sequence having at least about 90% identity to SEQ ID NOs: 630-645 and 1904-1933. In some cases, the NLS comprises a sequence having at least about 91% identity to SEQ ID NOs: 630-645 and 1904-1933. In some cases, the NLS comprises a sequence having at least about 92% identity to SEQ ID NOs: 630-645 and 1904-1933. In some cases, the NLS comprises a sequence having at least about 93% identity to SEQ ID NOs: 630-645 and 1904-1933. In some cases, the NLS comprises a sequence having at least about 94% identity to SEQ ID NOs: 630-645 and 1904-1933. In some cases, the NLS comprises a sequence having at least about 95% identity to SEQ ID NOs: 630-645 and 1904-1933. In some cases, the NLS comprises a sequence having at least about 96% identity to SEQ ID NOs: 630-645 and 1904-1933. In some cases, the NLS comprises a sequence having at least about 97% identity to SEQ ID NOs: 630-645 and 1904-1933. In some cases, the NLS comprises a sequence having at least about 98% identity to SEQ ID NOs: 630-645 and 1904-1933. In some cases, the NLS comprises a sequence having at least about 99% identity to SEQ ID NOs: 630-645 and 1904-1933. In some cases, the NLS comprises a sequence having 100% identity to SEQ ID NOs: 630-645 and 1904-1933.

TABLE 1
Example NLS Sequences that may be used with Cas effectors according to the
disclosure.

| | | SEQ ID |
|---|---|---|
| Source | NLS amino acid sequence | NO: |
| SV40 | PKKKRKV | 630 |
| nucleoplasmin bipartite NLS | KRPAATKKAGQAKKKK | 631 |
| c-myc NLS | PAAKRVKLD | 632 |
| c-myc NLS | RQRRNELKRSP | 633 |
| hRNPA1 M9 NLS | NQSSNFGPMKGGNFGGRSSGPYGGGGQYFA | 634 |
| | KPRNQGGY | |
| Importin-alpha IBB domain | RMRIZFKNKGKDTAELRRRRVEVSVELRKA | 635 |
| | KKDEQILKRRNV | |
| Myoma T protein | VSRKRPRP | 636 |
| Myoma T protein | PPKKARED | 637 |
| p53 | PQPKKKPL | 638 |
| mouse c-abl IV | SALIKKKKKMAP | 639 |
| influenza virus NS1 | DRLRR | 640 |
| influenza virus NS1 | PKQKKRK | 641 |
| Hepatitis virus delta antigen | RKLKKKIKKL | 642 |
| mouse Mx1 protein | REKKKFLKRR | 643 |
| human poly(ADP-ribose) | KRKGDEVDGVDEVAKKKSKK | 644 |
| polymerase | | |
| steroid hormone receptors | RKCLQAGMNLEARKTKK | 645 |
| (human) glucocorticoid | | |
| 5′ SV40 NLS | MAPKKKRKVGGGGS | 1904 |
| 3′ SV40 1 NLS | SGGAPKKKRKV | 1905 |
| 3′ SV40 2 NLS | SGGAPKKKRKV | 1906 |
| 3′ SV40 3 NLS | SGGAPKKKRKVSGGAPKKKRKVSGGAPKK | 1907 |
| | KRKV | |
| 5′ Nucleoplasmin NLS | MKRPAATKKAGQAKKKKGGGGS | 1908 |
| 3′ Nucleoplasmin 1 NLS | SGGKRPAATKKAGQAKKKK | 1909 |
| 3′ Nucleoplasmin 2 NLS | SGGKRPAATKKAGQAKKKKSGGKRP AATK | 1910 |
| | KAGQAKKKK | |
| 3′ Nucleoplasmin 3 NLS | SGGKRPAATKKAGQAKKKKSGGKRPAATK | 1911 |
| | KAGQAKKKKSGGKRPAATKKAGQAKKKK | |
| 5′ c-Myc NLS | MPAAKRVKLDGGGGS | 1912 |
| 3′ c-Myc 1 NLS | SGGPAAKRVKLD | 1913 |
| 3′ c-Myc 2 NLS | SGGPAAKRVKLDSGGPAAKRVKLD | 1914 |
| 3′ c-Myc 3 NLS | SGGPAAKRVKLDSGGPAAKRVKLDSGGP AA | 1915 |
| | KRVKLD | |
| Synthetic 5′ Class 2-1 NLS | MQAAKRPRTTGGGGS | 1916 |
| Synthetic 3′ Class 2-1 1 NLS | SGGQAAKRPRTT | 1917 |
| Synthetic 3′ Class 2-1 2 NLS | SGGQAAKRPRTTSGGQAAKRPRTT | 1918 |
| Synthetic 5′ Class 2-2 NLS | MRAAKRPRTTGGGGS | 1919 |
| Synthetic 3′ Class 2-2 1 NLS | SGGRAAKRPRTT | 1920 |
| Synthetic 3′ Class 2-2 2 NLS | SGGRAAKRPRTTSGGRAAKRPRTT | 1921 |
| Synthetic 5′ Class 2-2 NLS | MRAAKRPRTTGGGGS | 1922 |
| Synthetic 3′ Class 2-2 1 NLS | SGGRAAKRPRTT | 1923 |
| Synthetic 3′ Class 2-2 2 NLS | SGGRAAKRPRTTSGGRAAKRPRTT | 1924 |
| Synthetic 5′ Class 3 NLS | MAAAKRSWSMAFGGGGS | 1925 |
| Synthetic 3′ Class 3 1 NLS | MSGGAAAKRSWSMAF | 1926 |
| Synthetic 3′ Class 3 2 NLS | MSGGAAAKRSWSMAFMSGGAAAKRSWSM | 1927 |
| | AF | |
| Synthetic 5′ Class 4 NLS | MRAAKRKYFAVGGGGS | 1928 |
| Synthetic 3′ Class 4 1 NLS | MSGGRAAKRKYFAV | 1929 |
| Synthetic 3′ Class 4 2 NLS | MSGGRAAKRKYFAVMSGGRAAKRKYFAV | 1930 |
| 5′ HCV NLS | MPKPQRKTKRGGGGS | 1931 |
| 3′ HCV NLS | SGGPPRKKRTVV | 1932 |
| 3′ hRNP A1 NLS | SGGFGNYNNQSSNFGPMKGGNFGGRSSGPY | 1933 |

In some cases, the engineered nuclease system further comprises a single- or double-stranded DNA repair template. In some cases, the engineered nuclease system further comprises a single-stranded DNA repair template.

In some cases, the single- or double-stranded DNA repair template comprises from 5′ to 3′: a first homology arm comprising a sequence of at least 20 nucleotides 5′ to the target deoxyribonucleic acid sequence, a synthetic DNA sequence of at least 10 nucleotides, and a second homology arm comprising a sequence of at least 20 nucleotides 3′ to the target sequence.

In some cases, the first homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides. In some cases, the second homology arm comprises a sequence of at least 40, at least 50, at least 60, at least 70, at least 80, at least 90, at least 100, at least 110, at least 120, at least 130, at least 140, at least 150, at least 175, at least 200, at least 250, at least 300, at least 400, at least 500, at least 750, or at least 1000 nucleotides.

In some cases, the first and second homology arms are homologous to a genomic sequence of a prokaryote. In some cases, the first and second homology arms are homologous to a genomic sequence of a bacteria. In some cases, the first and second homology arms are homologous to a genomic sequence of a fungus. In some cases, the first and second homology arms are homologous to a genomic sequence of a eukaryote.

In some cases, the engineered nuclease system further comprises a DNA repair template comprising a double-stranded DNA segment flanked by one or two single-stranded DNA segments. In some embodiments, the single-stranded DNA segments are conjugated to the 5′ ends of the double-stranded DNA segment. In some embodiments, the double-stranded DNA segment is flanked by two single-stranded DNA segments. In some embodiments, the single-stranded DNA segments are conjugated to the 3′ ends of the double-stranded DNA segment. In some embodiments, the -stranded DNA segments have a length from 4 to 10 nucleotide bases. In some embodiments, the single-stranded DNA segments have a nucleotide sequence complementary to a sequence within the spacer sequence.

In some cases, the single-stranded DNA segments have a length from 1 to 15 nucleotide bases. In some cases, the single-stranded DNA segments have a length from 4 to 10 nucleotide bases. In some cases, the single-stranded DNA segments have a length of 4 nucleotide bases. In some cases, the single-stranded DNA segments have a length of 5 nucleotide bases. In some cases, the single-stranded DNA segments have a length of 6 nucleotide bases. In some cases, the single-stranded DNA segments have a length of 7 nucleotide bases. In some cases, the single-stranded DNA segments have a length of 8 nucleotide bases. In some cases, the single-stranded DNA segments have a length of 9 nucleotide bases. In some cases, the single-stranded DNA segments have a length of 10 nucleotide bases.

In some cases, the single-stranded DNA segments have a nucleotide sequence complementary to a sequence within the spacer sequence. In some cases, the double-stranded DNA sequence comprises a barcode, an open reading frame, an enhancer, a promoter, a protein-coding sequence, a miRNA coding sequence, an RNA coding sequence, or a transgene. In some embodiments, the double-stranded DNA sequence is flanked by a nuclease cut site. In some embodiments, the nuclease cut site comprises a spacer and a PAM sequence. In some embodiments, the PAM comprises a sequence of any one of SEQ ID NOs: 433, 435, 437, 439, 441, 443, 445, 447, 449, 451, 453, 455, 457, 459, 461, 463, 465, 467, 469, 471, 473, 475, and 646.

In some cases, the engineered nuclease system further comprises a source of Mg²⁺.

In some cases, the sequence is determined by a BLASTP, CLUSTALW, MUSCLE, or MAFFT algorithm, or a CLUSTALW algorithm with the Smith-Waterman homology search algorithm parameters. In some cases, the sequence is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

### Guide Polynucleotides

In some embodiments, the engineered nuclease system disclosed herein comprises an engineered guide polynucleotide, e.g., a guide ribonucleic acid (gRNA), a single gRNA, or a dual guide RNA.

In some embodiments, the engineered polynucleotide comprises a sequence having at least about 70% identity to any one of SEQ ID NOs: 333-335 and 355-357. In some embodiments, the engineered polynucleotide comprises a sequence having at least about 75% identity to any one of SEQ ID NOs: 333-335 and 355-357. In some embodiments, the engineered polynucleotide comprises a sequence having at least about 80% identity to any one of SEQ ID NOs: 333-335 and 355-357. In some embodiments, the engineered polynucleotide comprises a sequence having at least about 85% identity to any one of SEQ ID NOs: 333-335 and 355-357. In some embodiments, the engineered polynucleotide comprises a sequence having at least about 90% identity to any one of SEQ ID NOs: 333-335 and 355-357. In some embodiments, the engineered polynucleotide comprises a sequence having at least about 95% identity to any one of SEQ ID NOs: 333-335 and 355-357. In some embodiments, the engineered polynucleotide comprises a sequence having at least about 96% identity to any one of SEQ ID NOs: 333-335 and 355-357. In some embodiments, the engineered polynucleotide comprises a sequence having at least about 97% identity to any one of SEQ ID NOs: 333-335 and 355-357. In some embodiments, the engineered polynucleotide comprises a sequence having at least about 98% identity to any one of SEQ ID NOs: 333-335 and 355-357. In some embodiments, the engineered polynucleotide comprises a sequence having at least about 99% identity to any one of SEQ ID NOs: 333-335 and 355-357. In some embodiments, the engineered polynucleotide comprises 100% identity to any one of SEQ ID NOs: 333-335 and 355-357.

In some embodiments, the engineered polynucleotide comprises a sequence having at least about 70% identity to SEQ ID NO: 410-411. In some embodiments, the engineered polynucleotide comprises a sequence having at least about 75% identity to SEQ ID NO: 410-411. In some embodiments, the engineered polynucleotide comprises a sequence having at least about 80% identity to SEQ ID NO: 410-411. In some embodiments, the engineered polynucleotide comprises a sequence having at least about 85% identity to SEQ ID NO: 410-411. In some embodiments, the engineered polynucleotide comprises a sequence having at least about 90% identity to SEQ ID NO: 410-411. In some embodiments, the engineered polynucleotide comprises a sequence having at least about 95% identity to SEQ ID NO: 410-411. In some embodiments, the engineered polynucleotide comprises a sequence having at least about 96% identity to SEQ ID NO: 410-411. In some embodiments, the engineered polynucleotide comprises a sequence having at least about 97% identity to SEQ ID NO: 410-411. In some embodiments, the engineered polynucleotide comprises a sequence having at least about 98% identity to SEQ ID NO: 410-411. In some embodiments, the engineered polynucleotide comprises a sequence having at least about 99% identity to SEQ ID NO: 410-411. In some embodiments, the engineered polynucleotide comprises 100% identity to SEQ ID NO: 410-411.

In some embodiments, the engineered polynucleotide comprises a sequence having at least about 70% identity to any one of SEQ ID NOs: 346-347, 368-369, and 412-413. In some embodiments, the engineered polynucleotide comprises a sequence having at least about 75% identity to any one of SEQ ID NOs: 346-347, 368-369, and 412-413. In some embodiments, the engineered polynucleotide comprises a sequence having at least about 80% identity to any one of SEQ ID NOs: 346-347, 368-369, and 412-413. In some embodiments, the engineered polynucleotide comprises a sequence having at least about 85% identity to any one of SEQ ID NOs: 346-347, 368-369, and 412-413. In some embodiments, the engineered polynucleotide comprises a sequence having at least about 90% identity to any one of SEQ ID NOs: 346-347, 368-369, and 412-413. In some embodiments, the engineered polynucleotide comprises a sequence having at least about 95% identity to any one of SEQ ID NOs: 346-347, 368-369, and 412-413. In some embodiments, the engineered polynucleotide comprises a sequence having at least about 96% identity to any one of SEQ ID NOs: 346-347, 368-369, and 412-413. In some embodiments, the engineered polynucleotide comprises a sequence having at least about 97% identity to any one of SEQ ID NOs: 346-347, 368-369, and 412-413. In some embodiments, the engineered polynucleotide comprises a sequence having at least about 98% identity to any one of SEQ ID NOs: 346-347, 368-369, and 412-413. In some embodiments, the engineered polynucleotide comprises a sequence having at least about 99% identity to any one of SEQ ID NOs: 346-347, 368-369, and 412-413. In some embodiments, the engineered polynucleotide comprises 100% identity to any one of SEQ ID NOs: 346-347, 368-369, and 412-413.

In some embodiments, the engineered polynucleotide comprises a sequence having at least about 70% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 75% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 80% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 85% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 90% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 95% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 96% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 97% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 98% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises a sequence having at least about 99% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876. In some embodiments, the engineered polynucleotide comprises 100% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1697-1731, and 1876.

In some embodiments, the engineered polynucleotide comprises a sequence having at least about 70% identity to any one of SEQ ID NOs: 1091-1113, 1119-1120, and 1876. In some embodiments, the engineered polynucleotide comprises a sequence having at least about 75% identity to any one of SEQ ID NOs: 1091-1113, 1119-1120, and 1876. In some embodiments, the engineered polynucleotide comprises a sequence having at least about 80% identity to any one of SEQ ID NOs: 1091-1113, 1119-1120, and 1876. In some embodiments, the engineered polynucleotide comprises a sequence having at least about 85% identity to any one of SEQ ID NOs: 1091-1113, 1119-1120, and 1876. In some embodiments, the engineered polynucleotide comprises a sequence having at least about 90% identity to any one of SEQ ID NOs: 1091-1113, 1119-1120, and 1876. In some embodiments, the engineered polynucleotide comprises a sequence having at least about 95% identity to any one of SEQ ID NOs: 1091-1113, 1119-1120, and 1876. In some embodiments, the engineered polynucleotide comprises a sequence having at least about 96% identity to any one of SEQ ID NOs: 1091-1113, 1119-1120, and 1876. In some embodiments, the engineered polynucleotide comprises a sequence having at least about 97% identity to any one of SEQ ID NOs: 1091-1113, 1119-1120, and 1876. In some embodiments, the engineered polynucleotide comprises a sequence having at least about 98% identity to any one of SEQ ID NOs: 1091-1113, 1119-1120, and 1876. In some embodiments, the engineered polynucleotide comprises a sequence having at least about 99% identity to any one of SEQ ID NOs: 1091-1113, 1119-1120, and 1876. In some embodiments, the engineered polynucleotide comprises 100% identity to any one of SEQ ID NOs: 1091-1113, 1119-1120, and 1876.

In some embodiments, the engineered guide polynucleotide targets a gene in a cell. In some embodiments, the engineered guide polynucleotide targets a gene in a mammalian cell. In some embodiments, the mammalian cell is a pig, a cow, a goat, a sheep, a rodent, a rat, a mouse, a non-human primate, or a human cell. In some embodiments, the target gene or target locus is albumin, TRAC, AAVS1, or APOA1.

In some embodiments, the target gene is TRAC. In some embodiments, the guide polynucleotide targeting TRAC is encoded by any one of SEQ ID NOs: 767-798 or a sequence having at least 90%, 95%, 97%, 98%, or 99% sequence identity to any one of SEQ ID NOs: 767-798. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 767-798. In some embodiments, the guide polynucleotide is encoded by a sequence having at least about 80% identity to any one of SEQ ID NOs: 767-798. In some embodiments, the guide polynucleotide is encoded by a sequence having at least about 85% identity to any one of SEQ ID NOs: 767-798. In some embodiments, the guide polynucleotide is encoded by a sequence having at least about 90% identity to any one of SEQ ID NOs: 767-798. In some embodiments, the guide polynucleotide is encoded by a sequence having at least about 95% identity to any one of SEQ ID NOs: 767-798. In some embodiments, the guide polynucleotide is encoded by a sequence having at least about 96% identity to any one of SEQ ID NOs: 767-798. In some embodiments, the guide polynucleotide is encoded by a sequence having at least about 97% identity to any one of SEQ ID NOs: 767-798. In some embodiments, the guide polynucleotide is encoded by a sequence having at least about 98% identity to any one of SEQ ID NOs: 767-798. In some embodiments, the guide polynucleotide is encoded by a sequence having at least about 99% identity to any one of SEQ ID NOs: 767-798. In some embodiments, the guide polynucleotide is encoded by a sequence having 100% identity to any one of SEQ ID NOs: 767-798.

In some embodiments, the guide polynucleotide hybridizes or targets a sequence complementary to a target nucleic acid sequence within the TRAC gene or within an intron of the TRAC gene (e.g., SEQ ID NOs: 767-798). In some embodiments, the guide polynucleotide hybridizes or targets a sequence complementary to any one of SEQ ID NOs: 767-798 or a sequence having at least 90%, 95%, 97%, 98%, or 99% sequence identity to any one of SEQ ID NOs: 767-798. In some embodiments, the guide polynucleotide hybridizes or targets a sequence complementary to a sequence having at least about 80% identity to any one of SEQ ID NOs: 767-798. In some embodiments, the guide polynucleotide hybridizes or targets a sequence complementary to a sequence having at least about 85% identity to any one of SEQ ID NOS: 767-798. In some embodiments, the guide polynucleotide hybridizes or targets a sequence complementary to a sequence having at least about 90% identity to any one of SEQ ID NOS: 767-798. In some embodiments, the guide polynucleotide hybridizes or targets a sequence complementary to a sequence having at least about 95% identity to any one of SEQ ID NOS: 767-798. In some embodiments, the guide polynucleotide hybridizes or targets a sequence complementary to a sequence having at least about 96% identity to any one of SEQ ID NOS: 767-798. In some embodiments, the guide polynucleotide hybridizes or targets a sequence complementary to a sequence having at least about 97% identity to any one of SEQ ID NOs: 767-798. In some embodiments, the guide polynucleotide hybridizes or targets a sequence complementary to a sequence having at least about 98% identity to any one of SEQ ID NOs: 767-798. In some embodiments, the guide polynucleotide hybridizes or targets a sequence complementary to a sequence having at least about 99% identity to any one of SEQ ID NOS: 767-798. In some embodiments, the guide polynucleotide hybridizes or targets a sequence complementary to a sequence having 100% identity to any one of SEQ ID NOs: 767-798.

In some embodiments, the guide polynucleotide hybridizes or targets a sequence within the TRAC gene or within an intron of the TRAC gene (e.g., SEQ ID NOs: 799-830). In some embodiments, the guide polynucleotide hybridizes or targets a sequence according to any one of SEQ ID NOs: 799-830 or a sequence having at least 90%, 95%, 97%, 98%, or 99% sequence identity to any one of SEQ ID NOs: 799-830. In some embodiments, the guide polynucleotide hybridizes or targets a sequence having at least about 80% identity to any one of SEQ ID NOS: 799-830. In some embodiments, the guide polynucleotide hybridizes or targets a sequence having at least about 85% identity to any one of SEQ ID NOs: 799-830. In some embodiments, the guide polynucleotide hybridizes or targets a sequence having at least about 90% identity to any one of SEQ ID NOs: 799-830. In some embodiments, the guide polynucleotide hybridizes or targets a sequence having at least about 95% identity to any one of SEQ ID NOs: 799-830. In some embodiments, the guide polynucleotide hybridizes or targets a sequence having at least about 96% identity to any one of SEQ ID NOs: 799-830. In some embodiments, the guide polynucleotide hybridizes or targets a sequence having at least about 97% identity to any one of SEQ ID NOs: 799-830. In some embodiments, the guide polynucleotide hybridizes or targets a sequence having at least about 98% identity to any one of SEQ ID NOs: 799-830. In some embodiments, the guide polynucleotide hybridizes or targets a sequence having at least about 99% identity to any one of SEQ ID NOs: 799-830. In some embodiments, the guide polynucleotide hybridizes or targets a sequence having 100% identity to any one of SEQ ID NOs: 799-830.

In some embodiments, the target gene is APOA1. In some embodiments, the guide polynucleotide targeting APOA1 is encoded by any one of SEQ ID NOs: 831-904, 979-1021, 1219-1237, 1491-1506, and 1663-1669 or a sequence having at least 90%, 95%, 97%, 98%, or 99% sequence identity to any one of SEQ ID NOs: 831-904, 979-1021, 1219-1237, 1491-1506, and 1663-1669. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 831-904, 979-1021, 1219-1237, 1491-1506, and 1663-1669. In some embodiments, the guide polynucleotide is encoded by a sequence having at least about 80% identity to any one of SEQ ID NOs: 831-904, 979-1021, 1219-1237, 1491-1506, and 1663-1669. In some embodiments, the guide polynucleotide is encoded by a sequence having at least about 85% identity to any one of SEQ ID NOs: 831-904, 979-1021, 1219-1237, 1491-1506, and 1663-1669. In some embodiments, the guide polynucleotide is encoded by a sequence having at least about 90% identity to any one of SEQ ID NOs: 831-904, 979-1021, 1219-1237, 1491-1506, and 1663-1669. In some embodiments, the guide polynucleotide is encoded by a sequence having at least about 95% identity to any one of SEQ ID NOs: 831-904, 979-1021, 1219-1237, 1491-1506, and 1663-1669. In some embodiments, the guide polynucleotide is encoded by a sequence having at least about 96% identity to any one of SEQ ID NOs: 831-904, 979-1021, 1219-1237, 1491-1506, and 1663-1669. In some embodiments, the guide polynucleotide is encoded by a sequence having at least about 97% identity to any one of SEQ ID NOs: 831-904, 979-1021, 1219-1237, 1491-1506, and 1663-1669. In some embodiments, the guide polynucleotide is encoded by a sequence having at least about 98% identity to any one of SEQ ID NOs: 831-904, 979-1021, 1219-1237, 1491-1506, and 1663-1669. In some embodiments, the guide polynucleotide is encoded by a sequence having at least about 99% identity to any one of SEQ ID NOs: 831-904, 979-1021, 1219-1237, 1491-1506, and 1663-1669. In some embodiments, the guide polynucleotide is encoded by a sequence having 100% identity to any one of SEQ ID NOs: 831-904, 979-1021, 1219-1237, 1491-1506, and 1663-1669.

In some embodiments, the guide polynucleotide hybridizes or targets a sequence complementary to a target nucleic acid sequence within the APOA1 gene or within an intron of the APOA1 gene (e.g., SEQ ID NOs: 831-904, 979-1021, 1219-1237, 1491-1506, and 1663-1669). In some embodiments, the guide polynucleotide hybridizes or targets a sequence complementary to any one of SEQ ID NOs: 831-904, 979-1021, 1219-1237, 1491-1506, and 1663-1669 or a sequence having at least 90%, 95%, 97%, 98%, or 99% sequence identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 80% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 85% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 90% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 95% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 96% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 97% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 98% identity to any one of SEQ ID NOs: 831-904, 979-1021, 1219-1237, 1491-1506, and 1663-1669. In some embodiments, the guide polynucleotide hybridizes or targets a sequence complementary to a sequence having at least about 99% identity to any one of SEQ ID NOs: 831-904, 979-1021, 1219-1237, 1491-1506, and 1663-1669. In some embodiments, the guide polynucleotide hybridizes or targets a sequence complementary to a sequence having 100% identity to any one of SEQ ID NOs: 831-904, 979-1021, 1219-1237, 1491-1506, and 1663-1669.

In some embodiments, the guide polynucleotide hybridizes or targets a sequence within the APOA1 gene or within an intron of the APOA1 gene (e.g., SEQ ID NOs: 905-978, 1022-1064, 1238-1256, and 1670-1676). In some embodiments, the guide polynucleotide hybridizes or targets a sequence according to any one of SEQ ID NOs: 905-978, 1022-1064, 1238-1256, and 1670-1676 or a sequence having at least 90%, 95%, 97%, 98%, or 99% sequence identity to any one of SEQ ID NOs: 905-978, 1022-1064, 1238-1256, and 1670-1676. In some embodiments, the guide polynucleotide hybridizes or targets a sequence having at least about 80% identity to any one of SEQ ID NOs: 905-978, 1022-1064, 1238-1256, and 1670-1676. In some embodiments, the guide polynucleotide hybridizes or targets a sequence having at least about 85% identity to any one of SEQ ID NOs: 905-978, 1022-1064, 1238-1256, and 1670-1676. In some embodiments, the guide polynucleotide hybridizes or targets a sequence having at least about 90% identity to any one of SEQ ID NOs: 905-978, 1022-1064, 1238-1256, and 1670-1676. In some embodiments, the guide polynucleotide hybridizes or targets a sequence having at least about 95% identity to any one of SEQ ID NOs: 905-978, 1022-1064, 1238-1256, and 1670-1676. In some embodiments, the guide polynucleotide hybridizes or targets a sequence having at least about 96% identity to any one of SEQ ID NOs: 905-978, 1022-1064, 1238-1256, and 1670-1676. In some embodiments, the guide polynucleotide hybridizes or targets a sequence having at least about 97% identity to any one of SEQ ID NOs: 905-978, 1022-1064, 1238-1256, and 1670-1676. In some embodiments, the guide polynucleotide hybridizes or targets a sequence having at least about 98% identity to any one of SEQ ID NOs: 905-978, 1022-1064, 1238-1256, and 1670-1676. In some embodiments, the guide polynucleotide hybridizes or targets a sequence having at least about 99% identity to any one of SEQ ID NOs: 905-978, 1022-1064, 1238-1256, and 1670-1676. In some embodiments, the guide polynucleotide hybridizes or targets a sequence having 100% identity to any one of SEQ ID NOs: 905-978, 1022-1064, 1238-1256, and 1670-1676.

In some embodiments, the target gene is AAVS1. In some embodiments, the guide polynucleotide targeting AAVS1 is encoded by any one of SEQ ID NOs: 1257-1324, 1523-1562, 1677-1686, and 1753-1779, or a sequence having at least 90%, 95%, 97%, 98%, or 99% sequence identity to any one of SEQ ID NOs: 1257-1324, 1523-1562, 1677-1686 and 1753-1779. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1257-1324, 1523-1562, 1677-1686, and 1753-1779. In some embodiments, the guide polynucleotide is encoded by a sequence having at least about 80% identity to any one of SEQ ID NOs: 1257-1324, 1523-1562, 1677-1686, and 1753-1779. In some embodiments, the guide polynucleotide is encoded by a sequence having at least about 85% identity to any one of SEQ ID NOs: 1257-1324, 1523-1562, 1677-1686, and 1753-1779. In some embodiments, the guide polynucleotide is encoded by a sequence having at least about 90% identity to any one of SEQ ID NOs: 1257-1324, 1523-1562, 1677-1686, and 1753-1779. In some embodiments, the guide polynucleotide is encoded by a sequence having at least about 95% identity to any one of SEQ ID NOs: 1257-1324, 1523-1562, 1677-1686, and 1753-1779. In some embodiments, the guide polynucleotide is encoded by a sequence having at least about 96% identity to any one of SEQ ID NOs: 1257-1324, 1523-1562, 1677-1686, and 1753-1779. In some embodiments, the guide polynucleotide is encoded by a sequence having at least about 97% identity to any one of SEQ ID NOs: 1257-1324, 1523-1562, 1677-1686, and 1753-1779. In some embodiments, the guide polynucleotide is encoded by a sequence having at least about 98% identity to any one of SEQ ID NOs: 1257-1324, 1523-1562, and 1677-1686. In some embodiments, the guide polynucleotide is encoded by a sequence having at least about 99% identity to any one of SEQ ID NOs: 1257-1324, 1523-1562, 1677-1686, and 1753-1779. In some embodiments, the guide polynucleotide is encoded by a sequence having 100% identity to any one of SEQ ID NOs: 1257-1324, 1523-1562, 1677-1686, and 1753-1779.

In some embodiments, the guide polynucleotide hybridizes or targets a sequence complementary to a target nucleic acid sequence within the AAVS1 gene or within an intron of the AAVS1 gene (e.g., SEQ ID NOs: 1257-1324, 1523-1562, 1677-1686, and 1753-1779). In some embodiments, the guide polynucleotide hybridizes or targets a sequence complementary to any one of SEQ ID NOs: 1257-1324, 1523-1562, 1677-1686, and 1753-1779 or a sequence having at least 90%, 95%, 97%, 98%, or 99% sequence identity to any one of SEQ ID NOs: 1257-1324, 1523-1562, 1677-1686, and 1753-1779. In some embodiments, the guide polynucleotide hybridizes or targets a sequence complementary to a sequence having at least about 80% identity to any one of SEQ ID NOs: 1257-1324, 1523-1562, 1677-1686, and 1753-1779. In some embodiments, the guide polynucleotide hybridizes or targets a sequence complementary to a sequence having at least about 85% identity to any one of SEQ ID NOs: 1257-1324, 1523-1562, 1677-1686, and 1753-1779. In some embodiments, the guide polynucleotide hybridizes or targets a sequence complementary to a sequence having at least about 90% identity to any one of SEQ ID NOs: 1257-1324, 1523-1562, 1677-1686, and 1753-1779. In some embodiments, the guide polynucleotide hybridizes or targets a sequence complementary to a sequence having at least about 95% identity to any one of SEQ ID NOs: 1257-1324, 1523-1562, 1677-1686, and 1753-1779. In some embodiments, the guide polynucleotide hybridizes or targets a sequence complementary to a sequence having at least about 96% identity to any one of SEQ ID NOs: 1257-1324, 1523-1562, 1677-1686, and 1753-1779. In some embodiments, the guide polynucleotide hybridizes or targets a sequence complementary to a sequence having at least about 97% identity to any one of SEQ ID NOS: 1257-1324, 1523-1562, 1677-1686, and 1753-1779. In some embodiments, the guide polynucleotide hybridizes or targets a sequence complementary to a sequence having at least about 98% identity to any one of SEQ ID NOs: 1257-1324, 1523-1562, 1677-1686, and 1753-1779. In some embodiments, the guide polynucleotide hybridizes or targets a sequence complementary to a sequence having at least about 99% identity to any one of SEQ ID NOS: 1257-1324, 1523-1562, 1677-1686, and 1753-1779. In some embodiments, the guide polynucleotide hybridizes or targets a sequence complementary to a sequence having 100% identity to any one of SEQ ID NOs: 1257-1324, 1523-1562, 1677-1686, and 1753-1779.

In some embodiments, the guide polynucleotide hybridizes or targets a sequence within the AAVS1 gene or within an intron of the AAVS1 gene (e.g., SEQ ID NOs: 1325-1392, 1563-1602, 1687-1696, and 1780-1806). In some embodiments, the guide polynucleotide hybridizes or targets a sequence according to any one of SEQ ID NOs: 1325-1392, 1563-1602, 1687-1696, and 1780-1806 or a sequence having at least 90%, 95%, 97%, 98%, or 99% sequence identity to any one of SEQ ID NOs: 1325-1392, 1563-1602, 1687-1696, and 1780-1806. In some embodiments, the guide polynucleotide hybridizes or targets a sequence having at least about 80% identity to any one of SEQ ID NOs: 1325-1392, 1563-1602, 1687-1696, and 1780-1806. In some embodiments, the guide polynucleotide hybridizes or targets a sequence having at least about 85% identity to any one of SEQ ID NOs: 1325-1392, 1563-1602, 1687-1696, and 1780-1806. In some embodiments, the guide polynucleotide hybridizes or targets a sequence having at least about 90% identity to any one of SEQ ID NOs: 1325-1392, 1563-1602, 1687-1696, and 1780-1806. In some embodiments, the guide polynucleotide hybridizes or targets a sequence having at least about 95% identity to any one of SEQ ID NOs: 1325-1392, 1563-1602, 1687-1696, and 1780-1806. In some embodiments, the guide polynucleotide hybridizes or targets a sequence having at least about 96% identity to any one of SEQ ID NOs: 1325-1392, 1563-1602, 1687-1696, and 1780-1806. In some embodiments, the guide polynucleotide hybridizes or targets a sequence having at least about 97% identity to any one of SEQ ID NOs: 1325-1392, 1563-1602, 1687-1696, and 1780-1806. In some embodiments, the guide polynucleotide hybridizes or targets a sequence having at least about 98% identity to any one of SEQ ID NOs: 1325-1392, 1563-1602, 1687-1696, and 1780-1806. In some embodiments, the guide polynucleotide hybridizes or targets a sequence having at least about 99% identity to any one of SEQ ID NOs: 1325-1392, 1563-1602, 1687-1696, and 1780-1806. In some embodiments, the guide polynucleotide hybridizes or targets a sequence having 100% identity to any one of SEQ ID NOs: 1325-1392, 1563-1602, 1687-1696, and 1780-1806.

In some embodiments, the target gene is albumin. In some embodiments, the guide polynucleotide targeting albumin is encoded by any one of SEQ ID NOs: 1121-1169, 1393-1441, 1603-1632, 1887, 1889, 1891, and 1892-1893 or a sequence having at least 90%, 95%, 97%, 98%, or 99% sequence identity to any one of SEQ ID NOs: 1121-1169, 1393-1441, 1603-1632, 1887, 1889, 1891, and 1892-1893. In some embodiments, the guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1121-1169, 1393-1441, 1603-1632, 1887, 1889, 1891, and 1892-1893. In some embodiments, the guide polynucleotide is encoded by a sequence having at least about 80% identity to any one of SEQ ID NOs: 1121-1169, 1393-1441, 1603-1632, 1887, 1889, 1891, and 1892-1893. In some embodiments, the guide polynucleotide is encoded by a sequence having at least about 85% identity to any one of SEQ ID NOs: 1121-1169, 1393-1441, 1603-1632, 1887, 1889, 1891, and 1892-1893. In some embodiments, the guide polynucleotide is encoded by a sequence having at least about 90% identity to any one of SEQ ID NOs: 1121-1169, 1393-1441, 1603-1632, 1887, 1889, 1891, and 1892-1893. In some embodiments, the guide polynucleotide is encoded by a sequence having at least about 95% identity to any one of SEQ ID NOs: 1121-1169, 1393-1441, 1603-1632, 1887, 1889, 1891, and 1892-1893. In some embodiments, the guide polynucleotide is encoded by a sequence having at least about 96% identity to any one of SEQ ID NOs: 1121-1169, 1393-1441, 1603-1632, 1887, 1889, 1891, and 1892-1893. In some embodiments, the guide polynucleotide is encoded by a sequence having at least about 97% identity to any one of SEQ ID NOs: 1121-1169, 1393-1441, 1603-1632, 1887, 1889, 1891, and 1892-1893. In some embodiments, the guide polynucleotide is encoded by a sequence having at least about 98% identity to any one of SEQ ID NOs: 1121-1169, 1393-1441, 1603-1632, 1887, 1889, 1891, and 1892-1893. In some embodiments, the guide polynucleotide is encoded by a sequence having at least about 99% identity to any one of SEQ ID NOs: 1121-1169, 1393-1441, 1603-1632, 1887, 1889, 1891, and 1892-1893. In some embodiments, the guide polynucleotide is encoded by a sequence having 100% identity to any one of SEQ ID NOs: 1121-1169, 1393-1441, 1603-1632, 1887, 1889, 1891, and 1892-1893.

In some embodiments, the guide polynucleotide hybridizes or targets a sequence complementary to a target nucleic acid sequence within the albumin gene or within an intron of the albumin gene (e.g., SEQ ID NOs: 1121-1169, 1393-1441, 1603-1632, 1887, 1889, 1891, and 1892-1893). In some embodiments, the guide polynucleotide hybridizes or targets a sequence complementary to any one of SEQ ID NOs: 1121-1169, 1393-1441, 1603-1632, 1887, 1889, 1891, and 1892-1893 or a sequence having at least 90%, 95%, 97%, 98%, or 99% sequence identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 80% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 85% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 90% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 95% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 96% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 97% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 98% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having at least about 99% identity to any one of SEQ ID NOs: 1121-1169, 1393-1441, 1603-1632, 1887, 1889, 1891, and 1892-1893. In some embodiments, the guide polynucleotide hybridizes or targets a sequence complementary to a sequence having 100% identity to any one of SEQ ID NOs: 1121-1169, 1393-1441, 1603-1632, 1887, 1889, 1891, and 1892-1893.

In some embodiments, the guide polynucleotide hybridizes or targets a sequence within the albumin gene or within an intron of the albumin gene (e.g., SEQ ID NOs: 1170-1218, 1442-1490, 1633-1662, 1888, 1890, and 1994). In some embodiments, the guide polynucleotide hybridizes or targets a sequence according to any one of SEQ ID NOs: 1170-1218, 1442-1490, 1633-1662, 1888, 1890, and 1994 or a sequence having at least 90%, 95%, 97%, 98%, or 99% sequence identity to any one of SEQ ID NOs: 1170-1218, 1442-1490, 1633-1662, 1888, 1890, and 1994. In some embodiments, the guide polynucleotide hybridizes or targets a sequence having at least about 80% identity to any one of SEQ ID NOs: 1170-1218, 1442-1490, 1633-1662, 1888, 1890, and 1994. In some embodiments, the guide polynucleotide hybridizes or targets a sequence having at least about 85% identity to any one of SEQ ID NOs: 1170-1218, 1442-1490, 1633-1662, 1888, 1890, and 1994. In some embodiments, the guide polynucleotide hybridizes or targets a sequence having at least about 90% identity to any one of SEQ ID NOs: 1170-1218, 1442-1490, 1633-1662, 1888, 1890, and 1994. In some embodiments, the guide polynucleotide hybridizes or targets a sequence having at least about 95% identity to any one of SEQ ID NOs: 1170-1218, 1442-1490, 1633-1662, 1888, 1890, and 1994. In some embodiments, the guide polynucleotide hybridizes or targets a sequence having at least about 96% identity to any one of SEQ ID NOs: 1170-1218, 1442-1490, 1633-1662, 1888, 1890, and 1994. In some embodiments, the guide polynucleotide hybridizes or targets a sequence having at least about 97% identity to any one of SEQ ID NOs: 1170-1218, 1442-1490, 1633-1662, 1888, 1890, and 1994. In some embodiments, the guide polynucleotide hybridizes or targets a sequence having at least about 98% identity to any one of SEQ ID NOs: 1170-1218, 1442-1490, 1633-1662, 1888, 1890, and 1994. In some embodiments, the guide polynucleotide hybridizes or targets a sequence having at least about 99% identity to any one of SEQ ID NOs: 1170-1218, 1442-1490, 1633-1662, 1888, 1890, and 1994. In some embodiments, the guide polynucleotide hybridizes or targets a sequence having 100% identity to any one of SEQ ID NOs: 1170-1218, 1442-1490, 1633-1662, 1888, 1890, and 1994.

In some embodiments, the engineered guide polynucleotide is configured to form a complex with the endonuclease. In some cases, the engineered guide polynucleotide comprises a spacer sequence. In some cases, the spacer sequence is configured to hybridize to a target nucleic acid sequence. In some cases, the endonuclease is configured to bind to a protospacer adjacent motif (PAM) sequence.

In some embodiments, the engineered guide polynucleotide comprises a DNA-targeting segment comprising a nucleotide sequence that is complementary to a target sequence in a target nucleic acid site; and a protein-binding segment comprising two complementary stretches of nucleotides that hybridize to form a double-stranded RNA (dsRNA) duplex. In some embodiments, the two complementary stretches of nucleotides are covalently linked to one another with intervening nucleotides. In some embodiments, the engineered guide polynucleotide is capable of forming an endonuclease (e.g., a Class 2, Type V Cas endonuclease). In some embodiments, the DNA-targeting segment is positioned 3′ of both of the two complementary stretches of nucleotides.

In some cases, the endonuclease is not a Cpf1 or Cms1 endonuclease.

In some cases, the endonuclease is configured to bind to the engineered guide polynucleotide. In some cases, the Cas endonuclease is configured to bind to the engineered guide polynucleotide. In some cases, the class 2 Cas endonuclease is configured to bind to the engineered guide polynucleotide. In some cases, the class 2, type V Cas endonuclease is configured to bind to the engineered guide polynucleotide. In some cases, the class 2, type V, subtype Cas endonuclease is configured to bind to the engineered guide polynucleotide.

In some embodiments, the guide polynucleotide is configured to form a complex with the endonuclease. In some embodiments, the guide polynucleotide binds to the endonuclease to form a complex. In some embodiments, the guide polynucleotide binds (e.g., non-covalently through electrostatic interactions or hydrogen bonds) to the endonuclease to form a complex. In some embodiments, the guide polynucleotide is fused to the endonuclease to form a complex.

In some cases, the guide polynucleotide comprises a sequence complementary to a eukaryotic, fungal, plant, mammalian, or human genomic polynucleotide sequence. In some cases, the guide polynucleotide comprises a sequence complementary to a eukaryotic genomic polynucleotide sequence. In some cases, the guide polynucleotide comprises a sequence complementary to a fungal genomic polynucleotide sequence. In some cases, the guide polynucleotide comprises a sequence complementary to a plant genomic polynucleotide sequence. In some cases, the guide polynucleotide comprises a sequence complementary to a mammalian genomic polynucleotide sequence. In some cases, the guide polynucleotide comprises a sequence complementary to a human genomic polynucleotide sequence.

In some cases, the guide polynucleotide comprises a hairpin comprising at least 8 base-paired ribonucleotides. In some cases, the guide polynucleotide comprises a hairpin comprising at least 9 base-paired ribonucleotides. In some cases, the guide polynucleotide comprises a hairpin comprising at least 10 base-paired ribonucleotides. In some cases, the guide polynucleotide comprises a hairpin comprising at least 11 base-paired ribonucleotides. In some cases, the guide polynucleotide comprises a hairpin comprising at least 12 base-paired ribonucleotides.

In some cases, the guide polynucleotide is 30-250 nucleotides in length. In some cases, the guide polynucleotide is 42-44 nucleotides in length. In some cases, the guide polynucleotide is 42 nucleotides in length. In some cases, the guide polynucleotide is 43 nucleotides in length. In some cases, the guide polynucleotide is 44 nucleotides in length. In some cases, the guide polynucleotide is 85-245 nucleotides in length. In some cases, the guide polynucleotide is more than 90 nucleotides in length. In some cases, the guide polynucleotide is less than 245 nucleotides in length.

In some embodiments, the guide polynucleotide comprises synthetic nucleotides or modified nucleotides. In some embodiments, the guide polynucleotide comprises one or more inter-nucleoside linkers modified from the natural phosphodiester. In some embodiments, all of the inter-nucleoside linkers of the guide polynucleotide, or contiguous nucleotide sequence thereof, are modified. For example, in some embodiments, the inter nucleoside linkage comprises Sulphur(S), such as a phosphorothioate inter-nucleoside linkage.

In some embodiments, the present disclosure provides an engineered guide polynucleotide comprising a DNA-targeting segment. In some cases, the DNA-targeting segment comprises a nucleotide sequence that is complementary to a target sequence. In some cases, the target sequence is in a target DNA molecule. In some cases, the engineered guide polynucleotide comprises a protein-binding segment. In some cases, the protein-binding segment comprises two complementary stretches of nucleotides. In some cases, the two complementary stretches of nucleotides hybridize to form a double-stranded RNA (dsRNA) duplex. In some cases, the two complementary stretches of nucleotides are covalently linked to one another with intervening nucleotides. In some cases, the engineered guide ribonucleic acid polynucleotide is configured to form a complex with an endonuclease. In some cases, the endonuclease has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-325, 420-431, 476-624, or 629. In some cases, the complex targets the target sequence of the target DNA molecule. In some cases, the DNA-targeting segment is positioned 3′ of both of the two complementary stretches of nucleotides.

In some cases, the double-stranded RNA (dsRNA) duplex comprises at least 8 ribonucleotides. In some cases, the double-stranded RNA (dsRNA) duplex comprises at least 9 ribonucleotides. In some cases, the double-stranded RNA (dsRNA) duplex comprises at least 10 ribonucleotides. In some cases, the double-stranded RNA (dsRNA) duplex comprises at least 11 ribonucleotides. In some cases, the double-stranded RNA (dsRNA) duplex comprises at least 12 ribonucleotides.

In some embodiments, the guide polynucleotide comprises modifications to a ribose sugar or nucleobase. In some embodiments, the guide polynucleotide comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide polynucleotide comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′, or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than-H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide polynucleotide comprises one or more modified sugars. In some embodiments, the guide polynucleotide comprises only modified sugars. In certain embodiments, the guide polynucleotide comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide polynucleotide comprises both inter-nucleoside linker modifications and nucleoside modifications.

In some cases, the guide polynucleotide comprises a sequence complementary to a eukaryotic, fungal, plant, mammalian, or human genomic polynucleotide sequence. In some cases, the guide polynucleotide comprises a sequence complementary to a eukaryotic genomic polynucleotide sequence. In some cases, the guide polynucleotide comprises a sequence complementary to a fungal genomic polynucleotide sequence. In some cases, the guide polynucleotide comprises a sequence complementary to a plant genomic polynucleotide sequence. In some cases, the guide polynucleotide comprises a sequence complementary to a mammalian genomic polynucleotide sequence. In some cases, the guide polynucleotide comprises a sequence complementary to a human genomic polynucleotide sequence.

In some cases, the guide polynucleotide is 30-400 nucleotides in length. In some cases, the guide polynucleotide is 85-245 nucleotides in length. In some cases, the guide polynucleotide is more than 90 nucleotides in length. In some cases, the guide polynucleotide is less than 245 nucleotides in length. In some embodiments, the guide polynucleotide is 30, 40, 50, 60, 70, 80, 90, 100, 120, 140, 160, 180, 200, 220, 240, or more than 240 nucleotides in length. In some embodiments, the guide polynucleotide is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some embodiments, sequence is determined by a BLASTP, CLUSTALW, MUSCLE, or MAFFT algorithm, or a CLUSTALW algorithm with the Smith-Waterman homology search algorithm parameters. In some embodiments, sequence is determined by the BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

### MG Systems

Described herein, in certain embodiments, are engineered nuclease systems comprising an endonuclease comprising and an engineered guide polynucleotide. In some embodiments, the engineered guide polynucleotide comprises a tracrRNA. In some embodiments, the engineered guide polynucleotide comprises a guide nucleic acid (e.g., gRNA). In a polynucleotide when referring to a T, a T means U (Uracil) in RNA and T (Thymine) in DNA.

In some embodiments, the engineered nuclease system comprises an endonuclease comprising sequence having at least about 70% identity to any one of SEQ ID NOs: 6-14 and an engineered polynucleotide comprising a sequence having at least about 70% identity to any one of SEQ ID NOs: 333-335 and 355-357. In some embodiments, the engineered nuclease system comprises an endonuclease comprising sequence having at least about 75% identity to any one of SEQ ID NOs: 6-14 and an engineered polynucleotide comprising a sequence having at least about 75% identity to any one of SEQ ID NOs: 333-335 and 355-357. In some embodiments, the engineered nuclease system comprises an endonuclease comprising sequence having at least about 80% identity to any one of SEQ ID NOs: 6-14 and an engineered polynucleotide comprising a sequence having at least about 80% identity to any one of SEQ ID NOs: 333-335 and 355-357. In some embodiments, the engineered nuclease system comprises an endonuclease comprising sequence having at least about 85% identity to any one of SEQ ID NOs: 6-14 and an engineered polynucleotide comprising a sequence having at least about 85% identity to any one of SEQ ID NOs: 333-335 and 355-357. In some embodiments, the engineered nuclease system comprises an endonuclease comprising sequence having at least about 90% identity to any one of SEQ ID NOs: 6-14 and an engineered polynucleotide comprising a sequence having at least about 90% identity to any one of SEQ ID NOs: 333-335 and 355-357. In some embodiments, the engineered nuclease system comprises an endonuclease comprising sequence having at least about 95% identity to any one of SEQ ID NOs: 6-14 and an engineered polynucleotide comprising a sequence having at least about 95% identity to any one of SEQ ID NOs: 333-335 and 355-357. In some embodiments, the engineered nuclease system comprises an endonuclease comprising sequence having at least about 96% identity to any one of SEQ ID NOs: 6-14 and an engineered polynucleotide comprising a sequence having at least about 96% identity to any one of SEQ ID NOs: 333-335 and 355-357. In some embodiments, the engineered nuclease system comprises an endonuclease comprising sequence having at least about 97% identity to any one of SEQ ID NOs: 6-14 and an engineered polynucleotide comprising a sequence having at least about 97% identity to any one of SEQ ID NOs: 333-335 and 355-357. In some embodiments, the engineered nuclease system comprises an endonuclease comprising sequence having at least about 98% identity to any one of SEQ ID NOs: 6-14 and an engineered polynucleotide comprising a sequence having at least about 98% identity to any one of SEQ ID NOs: 333-335 and 355-357. In some embodiments, the engineered nuclease system comprises an endonuclease comprising sequence having at least about 99% identity to any one of SEQ ID NOs: 6-14 and an engineered polynucleotide comprising a sequence having at least about 99% identity to any one of SEQ ID NOs: 333-335 and 355-357. In some embodiments, the engineered nuclease system comprises an endonuclease comprising 100% identity to any one of SEQ ID NOs: 6-14 and an engineered polynucleotide comprising 100% identity to any one of SEQ ID NOs: 333-335 and 355-357.

In some embodiments, the engineered nuclease system comprises an endonuclease comprising sequence having at least about 70% identity to SEQ ID NO: 15 and an engineered polynucleotide comprising a sequence having at least about 70% identity to SEQ ID NO: 410-411. In some embodiments, the engineered nuclease system comprises an endonuclease comprising sequence having at least about 75% identity to SEQ ID NO: 15 and an engineered polynucleotide comprising a sequence having at least about 75% identity to SEQ ID NO: 410-411. In some embodiments, the engineered nuclease system comprises an endonuclease comprising sequence having at least about 80% identity to SEQ ID NO: 15 and an engineered polynucleotide comprising a sequence having at least about 80% identity to SEQ ID NO: 410-411. In some embodiments, the engineered nuclease system comprises an endonuclease comprising sequence having at least about 85% identity to SEQ ID NO: 15 and an engineered polynucleotide comprising a sequence having at least about 85% identity to SEQ ID NO: 410-411. In some embodiments, the engineered nuclease system comprises an endonuclease comprising sequence having at least about 90% identity to SEQ ID NO: 15 and an engineered polynucleotide comprising a sequence having at least about 90% identity to SEQ ID NO: 410-411. In some embodiments, the engineered nuclease system comprises an endonuclease comprising sequence having at least about 95% identity to SEQ ID NO: 15 and an engineered polynucleotide comprising a sequence having at least about 95% identity to SEQ ID NO: 410-411. In some embodiments, the engineered nuclease system comprises an endonuclease comprising sequence having at least about 96% identity to SEQ ID NO: 15 and an engineered polynucleotide comprising a sequence having at least about 96% identity to SEQ ID NO: 410-411. In some embodiments, the engineered nuclease system comprises an endonuclease comprising sequence having at least about 97% identity to SEQ ID NO: 15 and an engineered polynucleotide comprising a sequence having at least about 97% identity to SEQ ID NO: 410-411. In some embodiments, the engineered nuclease system comprises an endonuclease comprising sequence having at least about 98% identity to SEQ ID NO: 15 and an engineered polynucleotide comprising a sequence having at least about 98% identity to SEQ ID NO: 410-411. In some embodiments, the engineered nuclease system comprises an endonuclease comprising sequence having at least about 99% identity to SEQ ID NO: 15 and an engineered polynucleotide comprising a sequence having at least about 99% identity to SEQ ID NO: 410-411. In some embodiments, the engineered nuclease system comprises an endonuclease comprising 100% identity to SEQ ID NO: 15 and an engineered polynucleotide comprising 100% identity to SEQ ID NO: 410-411.

In some embodiments, the engineered nuclease system comprises an endonuclease comprising sequence having at least about 70% identity to any one of SEQ ID NOs: 16-29 and an engineered polynucleotide comprising a sequence having at least about 70% identity to any one of SEQ ID NOs: 346-347, 368-369, and 412-413. In some embodiments, the engineered nuclease system comprises an endonuclease comprising sequence having at least about 75% identity to any one of SEQ ID NOs: 16-29 and an engineered polynucleotide comprising a sequence having at least about 75% identity to any one of SEQ ID NOs: 346-347, 368-369, and 412-413. In some embodiments, the engineered nuclease system comprises an endonuclease comprising sequence having at least about 80% identity to any one of SEQ ID NOs: 16-29 and an engineered polynucleotide comprising a sequence having at least about 80% identity to any one of SEQ ID NOs: 346-347, 368-369, and 412-413. In some embodiments, the engineered nuclease system comprises an endonuclease comprising sequence having at least about 85% identity to any one of SEQ ID NOs: 16-29 and an engineered polynucleotide comprising a sequence having at least about 85% identity to any one of SEQ ID NOs: 346-347, 368-369, and 412-413. In some embodiments, the engineered nuclease system comprises an endonuclease comprising sequence having at least about 90% identity to any one of SEQ ID NOs: 16-29 and an engineered polynucleotide comprising a sequence having at least about 90% identity to any one of SEQ ID NOs: 346-347, 368-369, and 412-413. In some embodiments, the engineered nuclease system comprises an endonuclease comprising sequence having at least about 95% identity to any one of SEQ ID NOs: 16-29 and an engineered polynucleotide comprising a sequence having at least about 95% identity to any one of SEQ ID NOs: 346-347, 368-369, and 412-413. In some embodiments, the engineered nuclease system comprises an endonuclease comprising sequence having at least about 96% identity to any one of SEQ ID NOs: 16-29 and an engineered polynucleotide comprising a sequence having at least about 96% identity to any one of SEQ ID NOs: 346-347, 368-369, and 412-413. In some embodiments, the engineered nuclease system comprises an endonuclease comprising sequence having at least about 97% identity to any one of SEQ ID NOs: 16-29 and an engineered polynucleotide comprising a sequence having at least about 97% identity to any one of SEQ ID NOs: 346-347, 368-369, and 412-413. In some embodiments, the engineered nuclease system comprises an endonuclease comprising sequence having at least about 98% identity to any one of SEQ ID NOs: 16-29 and an engineered polynucleotide comprising a sequence having at least about 98% identity to any one of SEQ ID NOs: 346-347, 368-369, and 412-413. In some embodiments, the engineered nuclease system comprises an endonuclease comprising sequence having at least about 99% identity to any one of SEQ ID NOs: 16-29 and an engineered polynucleotide comprising a sequence having at least about 99% identity to any one of SEQ ID NOs: 346-347, 368-369, and 412-413. In some embodiments, the engineered nuclease system comprises an endonuclease comprising 100% identity to any one of SEQ ID NOs: 16-29 and an engineered polynucleotide comprising 100% identity to any one of SEQ ID NOs: 346-347, 368-369, and 412-413.

In some embodiments, the engineered nuclease system comprises an endonuclease comprising sequence having at least about 70% identity to any one of SEQ ID NOs: 30-150, 420-431, 476-624, and 629 and an engineered polynucleotide comprising a sequence having at least about 70% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 75% identity to any one of SEQ ID NOs: 30-150, 420-431, 476-624, and 629 and an engineered polynucleotide comprising a sequence having at least about 75% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 80% identity to any one of SEQ ID NOs: 30-150, 420-431, 476-624, and 629 and an engineered polynucleotide comprising a sequence having at least about 80% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 85% identity to any one of SEQ ID NOs: 30-150, 420-431, 476-624, and 629 and an engineered polynucleotide comprising a sequence having at least about 85% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 90% identity to any one of SEQ ID NOs: 30-150, 420-431, 476-624, and 629 and an engineered polynucleotide comprising a sequence having at least about 90% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 95% identity to any one of SEQ ID NOs: 30-150, 420-431, 476-624, and 629 and an engineered polynucleotide comprising a sequence having at least about 95% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 96% identity to any one of SEQ ID NOs: 30-150, 420-431, 476-624, and 629 and an engineered polynucleotide comprising a sequence having at least about 96% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 97% identity to any one of SEQ ID NOs: 30-150, 420-431, 476-624, and 629 and an engineered polynucleotide comprising a sequence having at least about 97% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 98% identity to any one of SEQ ID NOs: 30-150, 420-431, 476-624, and 629 and an engineered polynucleotide comprising a sequence having at least about 98% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising sequence having at least about 99% identity to any one of SEQ ID NOs: 30-150, 420-431, 476-624, and 629 and an engineered polynucleotide comprising a sequence having at least about 99% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731. In some embodiments, the engineered nuclease system comprises an endonuclease comprising 100% identity to any one of SEQ ID NOs: 30-150, 420-431, 476-624, and 629 and an engineered polynucleotide comprising 100% identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731.

In some embodiments, the engineered nuclease system comprises an endonuclease comprising sequence having at least about 70% identity to any one of SEQ ID NOs: 1065-1090 and 1114-1118 and an engineered polynucleotide comprising a sequence having at least about 70% identity to any one of SEQ ID NOs: 1091-1113, 1119-1120, and 1876. In some embodiments, the engineered nuclease system comprises an endonuclease comprising sequence having at least about 75% identity to any one of SEQ ID NOs: 1065-1090 and 1114-1118 and an engineered polynucleotide comprising a sequence having at least about 75% identity to any one of SEQ ID NOs: 1091-1113, 1119-1120, and 1876. In some embodiments, the engineered nuclease system comprises an endonuclease comprising sequence having at least about 80% identity to any one of SEQ ID NOs: 1065-1090 and 1114-1118 and an engineered polynucleotide comprising a sequence having at least about 80% identity to any one of SEQ ID NOs: 1091-1113, 1119-1120, and 1876. In some embodiments, the engineered nuclease system comprises an endonuclease comprising sequence having at least about 85% identity to any one of SEQ ID NOs: 1065-1090 and 1114-1118 and an engineered polynucleotide comprising a sequence having at least about 85% identity to any one of SEQ ID NOs: 1091-1113, 1119-1120, and 1876. In some embodiments, the engineered nuclease system comprises an endonuclease comprising sequence having at least about 90% identity to any one of SEQ ID NOs: 1065-1090 and 1114-1118 and an engineered polynucleotide comprising a sequence having at least about 90% identity to any one of SEQ ID NOs: 1091-1113, 1119-1120, and 1876. In some embodiments, the engineered nuclease system comprises an endonuclease comprising sequence having at least about 95% identity to any one of SEQ ID NOs: 1065-1090 and 1114-1118 and an engineered polynucleotide comprising a sequence having at least about 95% identity to any one of SEQ ID NOs: 1091-1113, 1119-1120, and 1876. In some embodiments, the engineered nuclease system comprises an endonuclease comprising sequence having at least about 96% identity to any one of SEQ ID NOs: 1065-1090 and 1114-1118 and an engineered polynucleotide comprising a sequence having at least about 96% identity to any one of SEQ ID NOs: 1091-1113, 1119-1120, and 1876. In some embodiments, the engineered nuclease system comprises an endonuclease comprising sequence having at least about 97% identity to any one of SEQ ID NOs: 1065-1090 and 1114-1118 and an engineered polynucleotide comprising a sequence having at least about 97% identity to any one of SEQ ID NOs: 1091-1113, 1119-1120, and 1876. In some embodiments, the engineered nuclease system comprises an endonuclease comprising sequence having at least about 98% identity to any one of SEQ ID NOs: 1065-1090 and 1114-1118 and an engineered polynucleotide comprising a sequence having at least about 98% identity to any one of SEQ ID NOs: 1091-1113, 1119-1120, and 1876. In some embodiments, the engineered nuclease system comprises an endonuclease comprising sequence having at least about 99% identity to any one of SEQ ID NOs: 1065-1090 and 1114-1118 and an engineered polynucleotide comprising a sequence having at least about 99% identity to any one of SEQ ID NOs: 1091-1113, 1119-1120, and 1876. In some embodiments, the engineered nuclease system comprises an endonuclease comprising 100% identity to any one of SEQ ID NOs: 1065-1090 and 1114-1118 and an engineered polynucleotide comprising 100% identity to any one of SEQ ID NOs: 1091-1113, 1119-1120, and 1876.

Further described herein, in some embodiments, are engineered nuclease systems comprising an endonuclease provided herein and a DNA methyltransferase. In some embodiments, the DNA methyltransferase binds non-covalently to the endonuclease. In some embodiments, the DNA methyltransferase is fused to the endonuclease in a single polypeptide. In some embodiments, the DNA methyltransferase comprises Dmnt3A or Dnmt3L. In some embodiments, the engineered nuclease system further comprises a KRAB domain. In some embodiments, the KRAB domain binds non-covalently to the endonuclease or the DNA methyltransferase. In some embodiments, the KRAB domain is covalently linked to the endonuclease or the DNA methyltransferase. In some embodiments, the KRAB domain is fused to the endonuclease or the DNA methyltransferase in a single polypeptide.

### Cells

Described herein, in certain embodiments, is a cell comprising the class 2, type V nuclease systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NS0), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a Streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof. In some embodiments, the primary cell is a T cell. In some embodiments, the primary cell is a hematopoietic stem cell (HSC).

### Delivery and Vectors

Disclosed herein, in some embodiments, are nucleic acid sequences encoding a MG90, MG91A, MG91B, MG91C, MG118, MG119, MG120, MG122, or MG126 system comprising a class 2, type V effector a gRNA, or a gene editing system disclosed herein.

In some embodiments, the nucleic acid encoding the MG90, MG91A, MG91B, MG91C, MG118, MG119, MG120, MG122, or MG126 system is a DNA, for example a linear DNA, a plasmid DNA, or a minicircle DNA. In some embodiments, the nucleic acid encoding the MG90, MG91A, MG91B, MG91C, MG118, MG119, MG120, MG122, or MG126 system is an RNA, for example a mRNA.

In some embodiments, the nucleic acid encoding the MG90, MG91A, MG91B, MG91C, MG118, MG119, MG120, MG122, or MG126 system is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3×FLAG, pSF-CMV-NEO-COOH-3×FLAG, pSF-CMV-PURO-NH2-GST-TEV, pSF-OXB20-COOH)-TEV-FLAG (R)-6His, pCEP4, pDEST27, pSF-CMV-Ub-KrYFP, pSF-CMV-FMDV-daGFP, pEF1a-mCherry-N1 vector, pEF1a-tdTomato vector, pSF-CMV-FMDV-Hygro, pSF-CMV-PGK-Puro, pMCP-tag (m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-21YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the class 2, type V effector or the genome editing system is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the class 2, type V effector or the genome editing system is introduced into the cell in any suitable way, either stably or transiently. In some embodiments, the class 2, type V effector or the genome editing system is transfected into the cell. In some embodiments, the cell is transduced or transfected with a nucleic acid construct that encodes the class 2, type V effector or the genome editing system. For example, a cell is transduced (e.g., with a virus encoding the class 2, type V effector or the genome editing system), or transfected (e.g., with a plasmid encoding the class 2, type V effector or the genome editing system) with a nucleic acid that encodes the class 2, type V effector or the genome editing system, or the translated the class 2, type V effector or the genome editing system. In some embodiments, the transduction is a stable or transient transduction. In some embodiments, cells expressing the class 2, type V effector or the genome editing system or containing the class 2, type V effector or the genome editing system are transduced or transfected with one or more gRNA molecules, for example, when the class 2, type V effector or the genome editing system comprises a CRISPR nuclease. In some embodiments, a plasmid expressing the class 2, type V effector or the genome editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Methods of Use

Gene editing systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

Described herein, in certain embodiments, are methods for modifying a target nucleic acid site comprising providing a class 2, type V effector or the genome editing system disclosed herein. In some embodiments, modifying the target nucleic acid site comprises binding, nicking, cleaving, marking, modifying, or transposing the target nucleic acid site. In some cases, the endonuclease induces a single-stranded break or a double-stranded break at or proximal to the target nucleic acid site. In some cases, the endonuclease induces a staggered single-stranded break within or 3′ to the target nucleic acid site.

In some embodiments, the target nucleic acid site is double-stranded. In some embodiments, the target nucleic acid site is single-stranded. In some cases, the target nucleic acid site comprises deoxyribonucleic acid (DNA). In some cases, the target nucleic acid site is double-stranded DNA. In some cases, the target nucleic acid site comprises ribonucleic acid (RNA). In some cases, the target nucleic acid site comprises genomic DNA, viral DNA, viral RNA, or bacterial DNA. In some cases, the target nucleic acid site is in vitro. In some cases, the target nucleic acid site is within a cell. In some cases, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is genome edited ex vivo. In some embodiments, the cell is genome edited in vivo.

In some embodiments, the methods are used to introduce a modification in the genome of a cell. In some embodiments, the modification is an insertion, deletion, or mutation. In some embodiments, the methods are used to introduce site-directed insertions, deletions, and/or mutations in the genome of a cell (for example an insertion and a mutation). In some embodiments, the methods are used in combination with a nucleic acid template to facilitate site-directed insertions into the genome of a cell.

In some embodiments, the methods are used for binding, cleaving, marking, or modifying a double-stranded deoxyribonucleic acid polynucleotide. In some embodiments, the methods comprise contacting the double-stranded deoxyribonucleic acid polynucleotide with an endonuclease. In some cases, the endonuclease is a Cas endonuclease. In some cases, the endonuclease is a class 2 Cas endonuclease. In some cases, the endonuclease is a class 2, type V Cas endonuclease. In some cases, the endonuclease is a class 2, type V, subtype Cas endonuclease. In some cases, the endonuclease is in complex with an engineered guide RNA. In some cases, the engineered guide RNA is configured to bind to the endonuclease. In some cases, the engineered guide RNA is configured to bind to the double-stranded deoxyribonucleic acid polynucleotide. In some cases, the engineered guide RNA is configured to bind to the endonuclease and to the double-stranded deoxyribonucleic acid polynucleotide. In some cases, the double-stranded deoxyribonucleic acid polynucleotide comprises a protospacer adjacent motif (PAM).

Described herein, in certain embodiments, are methods of modifying TRAC comprising contacting TRAC using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 767-798. In some embodiments, the engineered guide polynucleotide is encoded by any one of SEQ ID NOs: 767-798 or a sequence having at least 90%, 95%, 97%, 98%, or 99% sequence identity to any one of SEQ ID NOs: 767-798. In some embodiments, the engineered guide polynucleotide is configured to hybridize to a sequence or target a sequence complementary to a sequence comprising any one of SEQ ID NOs: 767-798 or a sequence having at least 90%, 95%, 97%, 98%, or 99% sequence identity to any one of SEQ ID NOs: 767-798. In some embodiments, the target nucleic acid sequence comprises a sequence having any one of SEQ ID NOs: 799-830. In some embodiments, the target nucleic acid sequence comprises a sequence having at least 90%, 95%, 97%, 98%, or 99% sequence identity to any one of SEQ ID NOs: 799-830.

Described herein, in certain embodiments, are methods of modifying APOA1 comprising contacting APOA1 using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 831-904, 979-1021, 1219-1237, 1491-1506, and 1663-1669. In some embodiments, the engineered guide polynucleotide is encoded by any one of SEQ ID NOs: 767-798 or a sequence having at least 90%, 95%, 97%, 98%, or 99% sequence identity to any one of SEQ ID NOs: 831-904, 979-1021, 1219-1237, 1491-1506, and 1663-1669. In some embodiments, the engineered guide polynucleotide is configured to hybridize to a sequence or target a sequence complementary to a sequence comprising any one of SEQ ID NOs: 831-904, 979-1021, 1219-1237, 1491-1506, and 1663-1669 or a sequence having at least 90%, 95%, 97%, 98%, or 99% sequence identity to any one of SEQ ID NOs: 831-904, 979-1021, 1219-1237, 1491-1506, and 1663-1669. In some embodiments, the target nucleic acid sequence comprises a sequence having any one of SEQ ID NOs: 905-978, 1022-1064, 1238-1256, and 1670-1676. In some embodiments, the target nucleic acid sequence comprises a sequence having at least 90%, 95%, 97%, 98%, or 99% sequence identity to any one of SEQ ID NOs: 905-978, 1022-1064, 1238-1256, and 1670-1676.

Described herein, in certain embodiments, are methods of modifying AAVS1 comprising contacting APOA1 using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1257-1324, 1523-1562, 1677-1686, and 1753-1779. In some embodiments, the engineered guide polynucleotide is encoded by any one of SEQ ID NOs: 1257-1324, 1523-1562, 1677-1686, and 1753-1779, or a sequence having at least 90%, 95%, 97%, 98%, or 99% sequence identity to any one of SEQ ID NOs: 1257-1324, 1523-1562, 1677-1686, and 1753-1779. In some embodiments, the engineered guide polynucleotide is configured to hybridize to a sequence or target a sequence complementary to a sequence comprising any one of SEQ ID NOs: 1257-1324, 1523-1562, 1677-1686, and 1753-1779, or a sequence having at least 90%, 95%, 97%, 98%, or 99% sequence identity to any one of SEQ ID NOs: 1257-1324, 1523-1562, 1677-1686, and 1753-1779. In some embodiments, the target nucleic acid sequence comprises a sequence having any one of SEQ ID NOs: 1325-1392, 1563-1602, 1687-1696, and 1780-1806. In some embodiments, the target nucleic acid sequence comprises a sequence having at least 90%, 95%, 97%, 98%, or 99% sequence identity to any one of SEQ ID NOs: 1325-1392, 1563-1602, 1687-1696, and 1780-1806.

Described herein, in certain embodiments, are methods of modifying albumin comprising contacting albumin using an engineered nuclease system comprising: a) an endonuclease comprising a sequence having at least 80% sequence identity to any one of SEQ ID NOs: 30-150, 420-431, 476-624, and 629; and b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1121-1169, 1393-1441, 1603-1632, 1887, 1889, 1891, and 1892-1893. In some embodiments, the engineered guide polynucleotide is encoded by any one of SEQ ID NOs: 1121-1169, 1393-1441, 1603-1632, 1887, 1889, 1891, and 1892-1893 or a sequence having at least 90%, 95%, 97%, 98%, or 99% sequence identity to any one of SEQ ID NOs: 1121-1169, 1393-1441, 1603-1632, 1887, 1889, 1891, and 1892-1893. In some embodiments, the engineered guide polynucleotide is configured to hybridize to a sequence or target a sequence complementary to a sequence comprising any one of SEQ ID NOs: 1121-1169, 1393-1441, 1603-1632, 1887, 1889, 1891, and 1892-1893 or a sequence having at least 90%, 95%, 97%, 98%, or 99% sequence identity to any one of SEQ ID NOs: 1121-1169, 1393-1441, 1603-1632, 1887, 1889, 1891, and 1892-1893. In some embodiments, the target nucleic acid sequence comprises a sequence having any one of SEQ ID NOs: 1170-1218, 1442-1490, 1633-1662, 1888, 1890, and 1994. In some embodiments, the target nucleic acid sequence comprises a sequence having at least 90%, 95%, 97%, 98%, or 99% sequence identity to any one of SEQ ID NOs: 1170-1218, 1442-1490, 1633-1662, 1888, 1890, and 1994.

Further described herein, in certain embodiments, are methods of manufacturing an engineered nuclease system described herein or components thereof. In some embodiments, the method comprises cultivating a host cell with the engineered nuclease system described herein or components thereof.

In some embodiments, the host cell is a bacterial cell. In some embodiments, the bacterial cell is Bifidobacterium longum, Bifidobacterium lactis, Bifidobacterium animalis, Bifidobacterium breve. Bifidobacterium infantis, Bifidobacterium adolescentis. Lactobacillus acidophilus, Lactobacillus casei, Lactobacillus paracasei, Lactobacillus salivarius, Lactobacillus reuteri, Lactobacillus rhamnosus, Lactobacillus johnsonii, Lactobacillus plantarum, Lactobacillus fermentum, Lactococcus lactis, Streptococcus thermophilus, Lactococcus lactis, Lactococcus diacetylactis, Lactococcus cremoris, Lactobacillus bulgaricus, Lactobacillus helveticus, Lactobacillus delbrueckii, or Escherichia coli. In some embodiments, the host cell is an E. coli cell. In some embodiments, the E. coli cell is a 2DE3 lysogen or a BL21 (DE3) strain. In some embodiments, the E. coli cell has an ompT lon genotype.

In some embodiments, the host cell is an E. coli cell. In some embodiments, the E. coli cell is a λDE3 lysogen or the E. coli cell is a BL21 (DE3) strain. In some embodiments, the E. coli cell has an ompT lon genotype.

In some embodiments, the open reading frame is operably linked to a promoter sequence. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof.

In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof.

In some embodiments, the open reading frame comprises a sequence encoding an affinity tag linked in-frame to a sequence encoding the engineered nuclease system described herein or components thereof. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, the affinity tag is linked in-frame to the sequence encoding the engineered nuclease system described herein or components thereof via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof.

In some embodiments, the open reading frame is codon-optimized for expression in the host cell. In some embodiments, the open reading frame is provided on a vector. In some embodiments, the open reading frame is integrated into a genome of the host cell.

In some embodiments, the present disclosure provides a culture comprising a host cell described herein in compatible liquid medium.

In some embodiments, the present disclosure provides a method of producing an engineered nuclease system described herein or components thereof, comprising cultivating a host cell described herein in compatible growth medium. In some embodiments, the method further comprises inducing expression of the engineered nuclease system described herein or components thereof by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the engineered nuclease system described herein or components thereof. In some embodiments, the IMAC affinity tag is linked in-frame to the sequence encoding the engineered nuclease system described herein or components thereof via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the engineered nuclease system described herein or components thereof. In some embodiments, the method further comprises performing subtractive IMAC affinity chromatography to remove the affinity tag from a composition comprising the engineered nuclease system described herein or components thereof.

### Kits

In some embodiments, this disclosure provides kits comprising one or more nucleic acid constructs encoding the various components of the class 2, type V effector and the genome editing system described herein, e.g., comprising a nucleotide sequence encoding the components of the class 2, type V effector and the genome editing system capable of modifying a target DNA sequence. In some embodiments, the nucleotide sequence comprises a heterologous promoter that drives expression of the RNA genome editing system components.

In some embodiments, the class 2, type V effector, the gRNA, or gene editing system comprising any combination thereof disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

### Example 1—A Method of Metagenomic Analysis for New Proteins

Metagenomic samples were collected from sediment, soil, and animals. Deoxyribonucleic acid (DNA) was extracted with a DNA mini-prep kit and sequenced. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on known Cas protein sequences including class 2 type V Cas effector proteins to identify new Cas effectors. Effector proteins identified by the search were aligned to known proteins to identify potential active sites. This metagenomic workflow resulted in the delineation of the MG90, MG91A, MG91B, MG91C, MG118, MG119, MG120, MG122, and MG126 families described herein.

### Example 2—Discovery of MG90, MG91A, MG91B, MG91C, MG118, MG119, MG120, MG122, and MG126 Families of CRISPR Systems

Analysis of the data from the metagenomic analysis of Example 1 revealed new clusters of previously undescribed putative CRISPR systems comprising 9 families (MG90, MG91A, MG91B, MG91C, MG118, MG119, MG120, MG122, and MG126). The corresponding protein and nucleic acid sequences for these new enzymes and their exemplary subdomains are presented as SEQ ID NOs: 1-325, 420-431, 476-624, or 629.

### Example 3—Template DNA for Transcription and Translation

E. coli codon optimized sequences of all MG VU and CasPhi nucleases were manufactured in a plasmid with a T7 promoter. Linear templates were amplified from the plasmids by PCR to include the T7 and nuclease sequence. Minimal array linear templates were amplified from sequences composed of a T7 promoter, native repeat, universal spacer, and native repeat, flanked by adapter sequences for amplification. The universal spacer matches the spacer in an 8N target library, where there are 8N mixed bases adjacent to the spacer for PAM determination. Three intergenic sequences near the ORF or CRISPR array were identified from the metagenomic contigs and ordered as gBlocks with flanking adapter sequences for amplification.

### Example 4—In Vitro Transcription of crRNA, Minimal Arrays, and sgRNA

RNA was produced by in vitro transcription using an RNA Synthesis Kit and purified using an RNA Cleanup Kit. Templates for T7 transcription varied. For crRNA, DNA oligos were designed with a T7 promoter, trimmed native repeat, and universal spacer. For minimal arrays the same templates as described above were used. For sgRNA, DNA ultramers were designed with a T7 promoter, trimmed tracrRNA, GAAA tetraloop, trimmed native repeat, and universal spacer. Minimal array templates were amplified with adapter primers. The crRNA and sgRNA templates were ordered as reverse complements and annealed with a primer with the T7 promoter sequence in 1× duplex buffer at 95° C. for two minutes followed by cooling to 22° C. at 0.1° C./second to produce a hybrid ds/ssDNA substrate suitable for transcription. After transcription, but prior to cleaning, each reaction was treated with DNAse I and incubated at 37° C. for 15 minutes. All transcription products were verified for yield and purity via RNA electrophoresis or via a denaturing urea PAGE gel.

### Example 5—TXTL Expression

Nucleases, intergenic sequences, and minimal arrays were expressed in transcription-translation reaction mixtures. The final reaction mixtures contained 5 nM nuclease DNA template, 12 nM intergenic DNA template, 15 nM minimal array DNA template, 0.1 nM pTXTL-P70a-T7map, and 1× of Master Mix. The reactions were incubated at 29° C. for 16 hours then stored at 4° C.

### Example 6—Protein Expressions

10 nM of nuclease PCR templates were expressed at 37° C. for 3 hours with an in vitro protein synthesis kit for cleavage with in vitro transcribed RNA. These reactions were used to test in vitro cleavage with 50 nM sgRNA or minimal array RNA following the same procedure as described in the cleavage reactions section.

### Example 7—E. coli Expressions

Plasmids encoding the effector, intergenic sequence from the genomic contig, native repeat, and universal spacer sequences with a T7 promoter were transformed into BL21 DE3 or T7 Express lysY/Iq and cultured at 37° C. in 60 mL terrific broth media supplemented with 100 μg/mL of ampicillin. Expression was induced with 0.4 mM IPTG after cultures reached OD_{600 nm} of 0.5 and incubated at 16° C. overnight. 25 mL of cells were pelleted by centrifugation and resuspended in 1.5 mL of lysis buffer (20 mM Tris-HCl, 500 mM NaCl, 1 mM TCEP, 5% glycerol, 10 mM MgCl₂ pH 7.5 with Protease Inhibitor). Cells were then lysed by sonication. Supernatant and cell debris were separated by centrifugation.

### Example 8—Cleavage Reactions

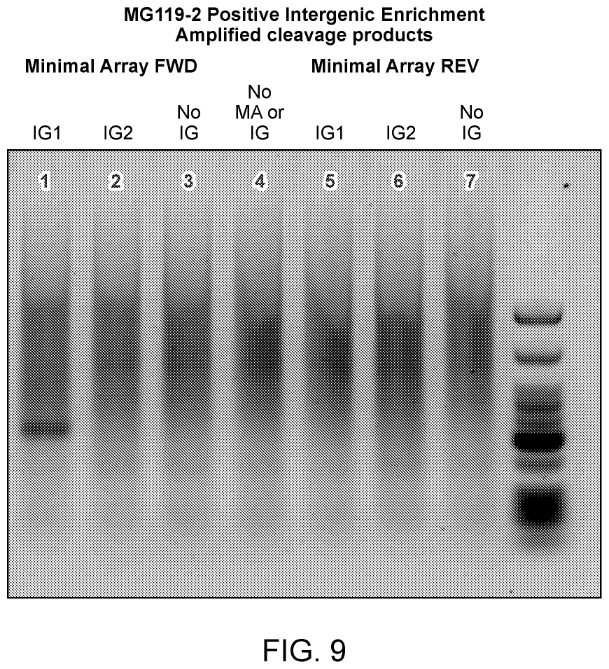

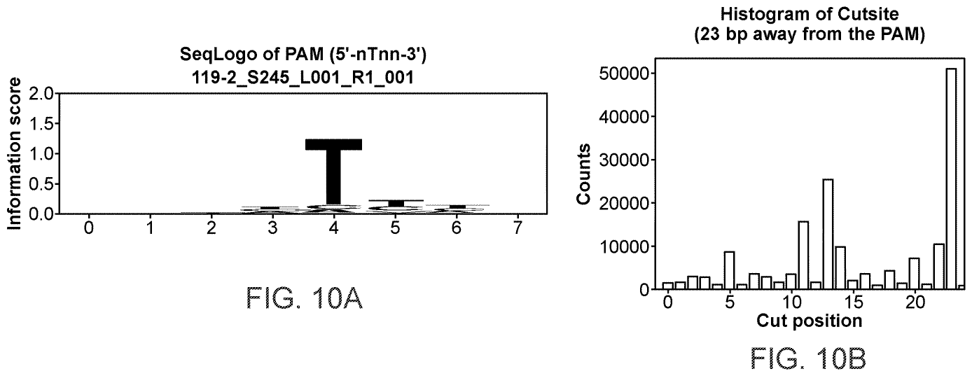

Plasmid library DNA cleavage reactions were carried out by mixing 5 nM of the target library, a 5-fold dilution of protein expressions, 10 nM Tris-HCl, 10 nM MgCl₂, and 100 mM NaCl at 37° C. for 2 hours. For reactions with E. coli expressions, 10 μL of the clarified lysate was added. Reactions were stopped and cleaned with PCR clean up beads and eluted in Tris EDTA pH 8.0 buffer. 3 nM of the cleavage product ends were blunted with 3.33 μM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, 20 U/μL T4 DNA ligase at room temperature for 20 minutes. The ligated products were amplified by PCR with NGS primers and sequenced by NGS to obtain the PAM. The in vitro activity of MG119-2 is depicted in FIG. 9, while the PAM determination for MG119-2 is depicted in FIG. 10.

### Example 9—RNAseq Library Prep of Intergenic Enrichment from TXTL and E. coli Lysates

RNA is extracted and cell lysate expressions following the RNA Miniprep Kit and eluted in 30-50 μL of water. The total concentration of the transcripts were measured.

100 ng-1 μg of total RNA from each sample were prepped for RNA sequencing. Amplicons between 150-300 bp were quantified by and pooled to a final concentration of 4 nM. A final concentration of 12.5 μM was loaded into a sequencing kit and sequenced for 176 total cycles. The RNAseq reads were used to identify the tracr sequence of the genes.

### Example 10—Predicted RNA Folding

Predicted RNA folding of the active single RNA sequence was computed at 37° C. The shading of the bases corresponds to the probability of base pairing of that base.

### Example 11—In Vitro Cleavage Efficiency

The protein is expressed in E. coli protease deficient B strain under T7 inducible promoter, the cells are lysed using sonication, and the His-tagged protein of interest is purified using Ni-NTA affinity chromatography on a FPLC. Purity is determined using densitometry in of the protein bands resolved on SDS-PAGE and Coomassie stained acrylamide gels. The protein is desalted in a storage buffer composed of 50 mM Tris-HCl, 300 mM NaCl, 1 mM TCEP, 5% glycerol; pH 7.5 and stored at −80° C.

A target DNA is constructed that contains a spacer sequence and the PAM determined via NGS. In the case of degenerate bases in the PAM a single representative PAM is chosen for testing. The target DNA is 2200 bp of linear DNA derived from a plasmid via PCR amplification. The PAM and spacer are located 700 bp from one end. Successful cleavage results in fragments of 700 and 1500 bp.

The target DNA, in vitro-transcribed single RNA, and purified recombinant protein are combined in cleavage buffer (10 mM Tris, 100 mM NaCl, 10 mM MgCl₂) with an excess of protein and RNA and incubated for 5′ to 3 hours, usually 1 hr. The reaction is stopped via addition of RNAse A and incubation at 60°. The reaction is resolved on a 1.2% TAE agarose gel and the fraction of cleaved target DNA is quantified in imaging software.

### Example 12—Activity in E. coli

For testing of nuclease activity in bacterial cells, strains are constructed with genome sequences containing the target spacer and corresponding PAM sequence specific to the enzyme of interest. Engineered strains are then transformed with the nuclease of interest and transformants are then subsequently made chemocompetent and transformed with 50 ng of single guides either specific to the target sequence, on target, or nonspecific to the target, off target. After heat shock, transformations are recovered in SOC for 2 hrs at 37° C., and nuclease efficiency is determined by a 5-fold dilution series grown on induction media. Colonies are quantified from the dilution series in triplicate.

### Example 13—Activity in Mammalian Cells

To show targeting and cleavage activity in mammalian cells, the protein sequences are cloned into 2 mammalian expression vectors, one with a C-terminal SV40 NLS and a 2A-GFP tag and one with no GFP tag and 2 NLS sequences, one on the N-terminus and one on the C-terminus. Alternative NLS sequences that can also be used. The DNA sequence for the protein can be the native sequence, the E. coli codon optimized sequence, or the mammalian codon optimized sequence. The single guide polynucleotide sequence with a gene target of interest is also cloned into a mammalian expression vector. The two plasmids are co-transfected into HEK293T cells. 72 hr after co-transfection of the expression plasmid and a sgRNA targeting plasmid into HEK293T cells, the DNA is extracted and used for the preparation of an NGS-library. Percent NHEJ is measured via indels in the sequencing of the target site to demonstrate the targeting efficiency of the enzyme in mammalian cells. At least 10 different target sites are chosen for testing each protein's activity.

### Example 14—Characterization of Compact Type V Nucleases in the MG119 Family

### In Silico Identification of Compact Type V Nucleases in the MG119 Family

Discovery of predicted proteins related to nuclease sequences in the MG119 family of compact type V nucleases was based on homology searches. Searches were performed and Type V nuclease sequence hits were retained if they met the following criteria: (i) the hmmsearch e-value was ≤10-5, (ii) the genes encoding the nuclease were within 1 kb from a CRISPR array, and (iii) the amino acid sequence length ranged between 350 and 700 aa. Sequences at 100% amino acid identity were clustered, with coverage mode 1 and 80% coverage of the target sequence. Sequence representatives were chosen to build a multiple sequence alignment a phylogenetic tree was built. Careful examination of individual clades on the phylogenetic tree, including the nuclease gene's genomic context, led to the identification of several compact type V nuclease sequences in the MG119 family (SEQ ID NOs: 476-624 and 629).

In Vitro Characterization to Identify Putative tracrRNAs

To identify putative tracrRNA sequences, e.g., for nuclease MG119-2, adjacent intergenic sequences and a minimal array were expressed in transcription-translation reaction mixtures using cell-free expression reactions (TXTL). The final reaction mixtures contained 5 nM nuclease DNA template, 12 nM intergenic DNA template, 15 nM minimal array DNA template, 0.1 nM pTXTL-P70a-T7rnap, and 1× of Master Mix. The reactions were incubated at 29° C. for 16 hours, then stored at 4° C.

Ribonucleoprotein complexes were tested via in vitro cleavage reactions. Plasmid DNA library cleavage reactions were carried out by mixing 5 nM of the target plasmid DNA library representing all possible 8N PAMs, a 5-fold dilution of the TXTL expressions, 10 nM Tris-HCl, 10 nM MgCl₂ and, 100 mM NaCl at 37° C. for 2 hours. Reactions were stopped and cleaned with PCR clean up beads and eluted in Tris EDTA pH 8.0 buffer.

To obtain the PAM sequences, 3 nM of the cleavage product ends were blunted with 3.33 AM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, and 20 U/μL T4 DNA ligase at room temperature for 20 minutes. The ligated products were amplified by PCR with NGS primers and sequenced by NGS.

To obtain the sequence of the tracrRNA and the crRNA, RNA was extracted from TXTL lysate following the RNA Miniprep Kit and eluted in 30-50 μL of water. 100 ng-1 μg of total RNA from each sample were prepped for RNA sequencing using a Small RNA Library Prep Set. Amplicons between 150-300 bp were quantified and pooled to a final concentration of 4 nM. A final concentration of 12.5 μM was loaded into a sequencing kit and sequenced for 176 total cycles. The RNAseq reads were used to identify the tracr sequence of the genes by mapping back to the original sequences.

In Silico Identification of tracrRNA Sequences

To identify additional non-coding regions containing potential tracrRNAs, the sequence of the active tracrRNA was mapped to other contigs containing nucleases in the same nuclease family (e.g. MG119-1 and MG119-3). The newly identified sequences were used to generate covariance models to predict additional tracrRNAs. Covariance models were built from a multiple sequence alignment (MSA) of the active and predicted tracrRNA sequences. The secondary structure of the MSA was obtained, and the covariance models were built. Other contigs containing candidate nucleases were searched using the covariance models. TracrRNA candidates were tested in vitro (see below), and in an iterative process, sequences from active candidates were used to improve the covariance models and search for additional tracrRNAs in the intergenic regions associated with other nuclease candidates.

sgRNA Design

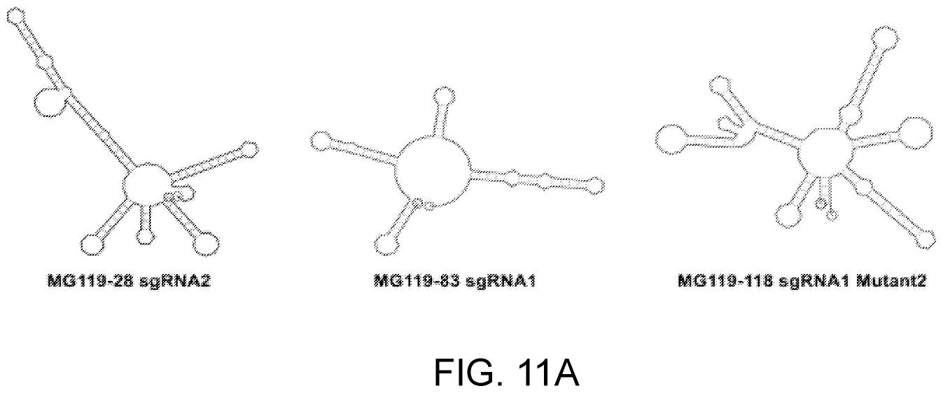

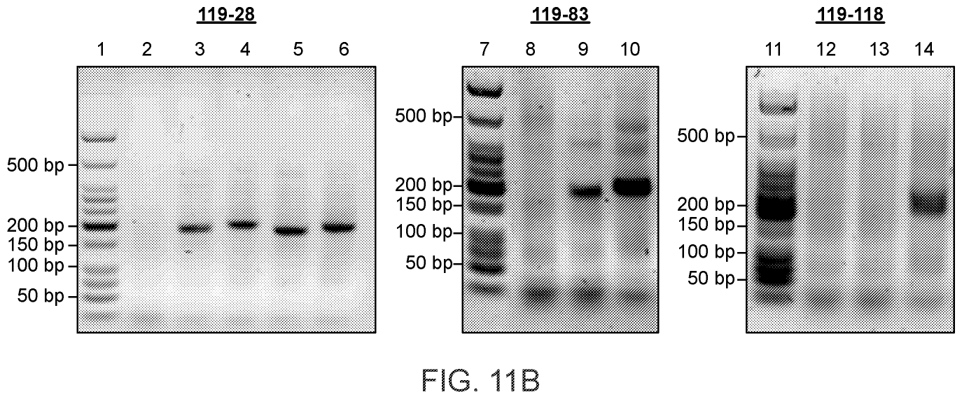

Predicted tracrRNAs obtained from the covariance models and their associated CRISPR repeat sequence were modified to generate sgRNAs (FIG. 11A) as follows: the 3′ end of the predicted tracrRNA sequence as well as the 5′ end of the repeat sequence were trimmed, and then connected with a GAAA tetraloop.

### In Vitro Cleavage Reactions to Confirm Nuclease Activity and Enable PAM Determination

5 nM of nuclease amplified DNA templates and 25 nM sgRNA amplified DNA templates (including one of the spacer sequences listed in Table 2) were expressed at 37° C. for 3 hours with an in vitro protein expression system. Plasmid library DNA cleavage reactions were carried out by mixing 5 nM of the target library representing all possible 8N PAMs, a 5-fold dilution of in vitro protein expressions, 10 mM Tris-HCl pH 7.9, 10 mM MgCl₂, 100 μg/mL BSA, and 50 mM NaCl at 37° C. for 2 hours. Reactions were stopped and cleaned with PCR clean up beads and eluted in Tris EDTA pH 8.0 buffer. 3 nM of the cleavage product ends were blunted with 3.33 μM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, and 20 U/μL T4 DNA ligase at room temperature for 20 minutes. The ligated products were amplified by PCR with NGS primers and sequenced by NGS to obtain the PAM. Active proteins that successfully cleaved the PAM library yielded a band around 188 or 205 bp in an agarose gel, depending on which target site was encoded in the sgRNA (FIG. 11B).

TABLE 2
Spacer sequences for tested guides

| Code | Sequence |
|---|---|
| U67 spacer | GTCGAGGCTTGCGACGTGGT |
| | (SEQ ID NO: 1877) |
| U40 spacer | TGGAGATATCTTGAACCTTG |
| | (SEQ ID NO: 1878) |

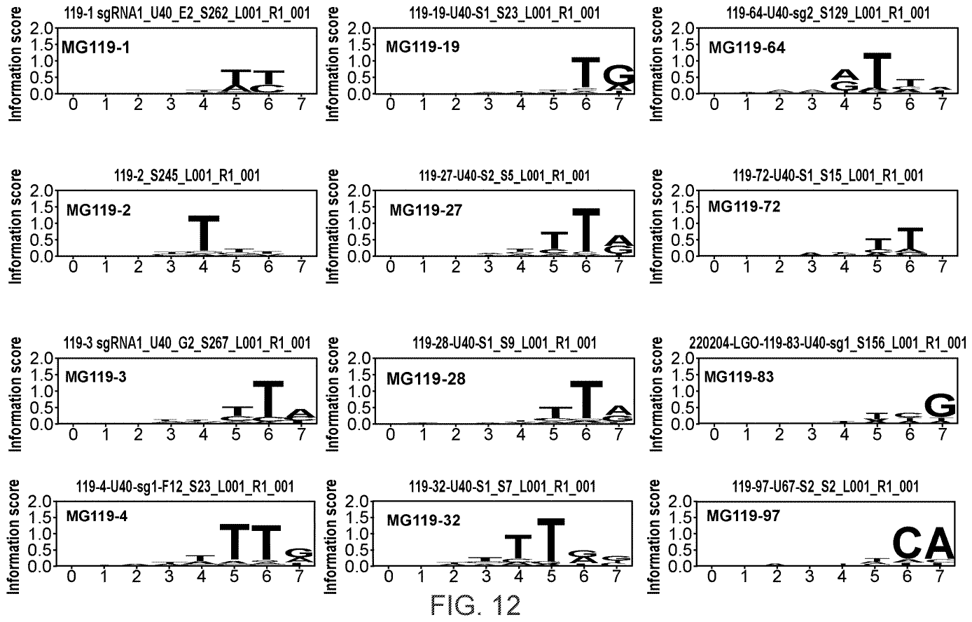

The PAMs recognized by MG119 nucleases are shown as sequence logos made with Seqlog maker (FIG. 12). The preferred cut position on target strand of the protospacer sequence complementary to the U40 spacer is listed in Table 3.

TABLE 3
MG119 nucleases preferred cutsites in the protospacer sequence

| Nuclease | SgRNA | Cutsite |
|---|---|---|
| 119-1 | MG119-1_sgRNA1 | 20 & 23 |
| 119-2 | MG119-2_sgRNA1_Mutant1 | 22 |
| 119-3 | MG119-3_sgRNA1_Mutant1 | 22-23 |
| 119-4 | MG119-4_sgRNA1 | 22-23 |
| 119-10 | MG119-10_sgRNA1 | 22-23 |
| 119-19 | MG119-19_sgRNA1 | 23 |
| 119-27 | MG119-27_sgRNA2_Mutant2 | 22-23 |
| 119-28 | MG119-28_sgRNA2 | 22-23 |
| 119-32 | MG119-32_sgRNA1 | 23 |
| 119-54 | MG119-54_sgRNA1 | 22 |
| 119-64 | MG119-64_sgRNA2 | 20 |
| 119-72 | MG119-72_sgRNA1 | 23 |
| 119-83 | MG119-83_sgRNA1 | 23 |
| 119-97 | MG119-97_sgRNA1_Mutant1 | 22 |
| 119-109 | MG119-109_sgRNA1 | 24-25 |
| 119-118 | MG119-118_sgRNA1_Mutant2 | 23 |
| 119-121 | MG119-121_sgRNA1_Mutant1 | 20 & 22 |
| 119-125 | MG119-125_sgRNA1 | 22-23 |
| 119-128 | MG119-128_sgRNA2_Mutant1 | 22 |
| 119-129 | MG119-129_sgRNA1_Mutant1 | 22-23 |
| 119-133 | MG119-133_sgRNA1_Mutant1 | 22 |
| 119-136 | MG119-136_sgRNA1_Mutant2 | 23 |
| 119-137 | MG119-137_sgRNA1 | 22-23 |

### Protein Expression and Purification

Isolating pure and functional proteins is essential for extensive in vitro analysis of biochemical properties and mechanistic studies. The expression and purification of MG119 candidates was optimized to obtain proteins of sufficient quantity and quality for such characterizations. All constructs were expressed in E. coli. Constructs were expressed in either the pMGB expression vector (MBP-fused), the pMGBA expression vector (no fusion protein), or both.

### Protein Expression

Protein expression protocols for pMGB and pMGBA constructs are identical. Cultures were grown at 37° C. in 2×YT media (1.6% tryptone, 1% yeast extract, 0.5% NaCl) or TB media with 100 μg/L Carbenicillin. At OD600≈0.8-1.2, cultures were induced with 0.5 mM IPTG and incubated at 18° C. overnight or 24° C. for 4-6 hrs, depending on construct. Cultures were then harvested by centrifugation at 6,000×g for 10 min, and pellets were resuspended in Nickel_A Buffer (50 mM Tris pH 7.5, 750 mM NaCl, 10 mM MgCl₂, 20 mM imidazole, 0.5 mM EDTA, 5% glycerol, 0.5 mM TCEP)+protease inhibitors (Protease Inhibitor Tablets, EDTA-free) and stored at −80° C.

Protein Purification—pMGBA Expression Vector

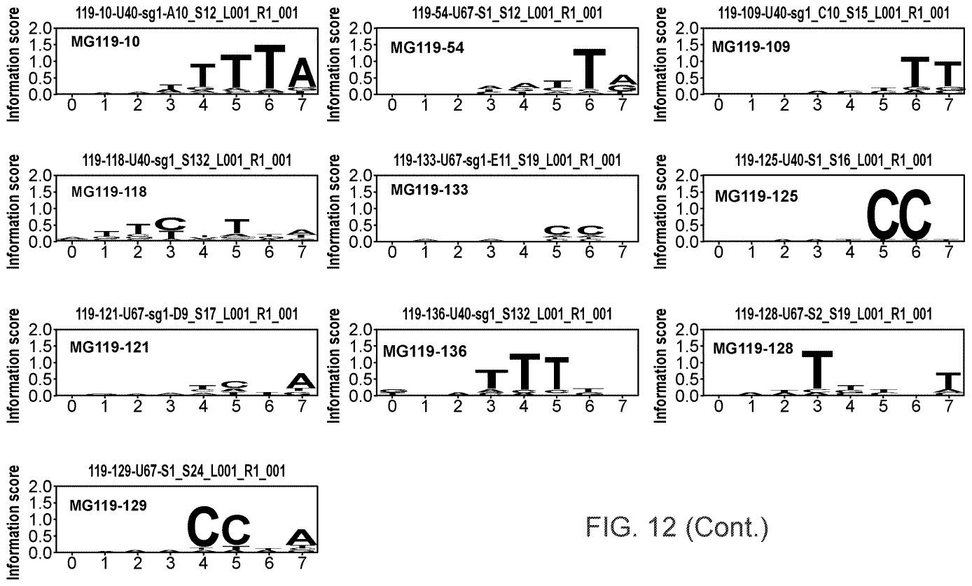

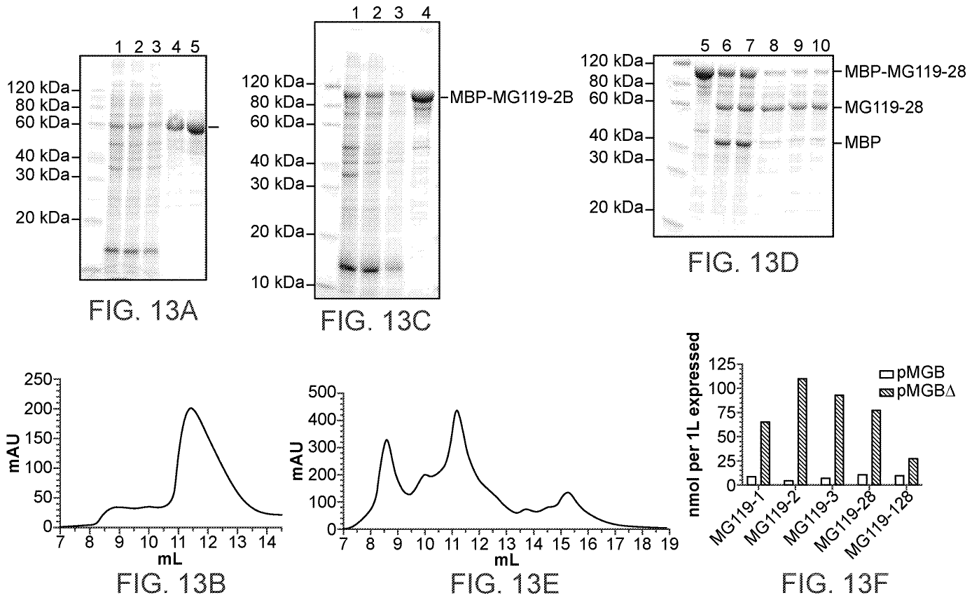

Proteins expressed in this vector have the following sequence architecture: 6×His-(GS)2-PSP-nucleoplasmin bipartite NLS-(GGS) 1-(GS)1-MG119-X-(GGS) 3-SV40 NLS (Table 5). Proteins expressed in this vector are denoted MG119-XA. Cell pellets were thawed and the volume supplemented to 120 mL with Cf=0.5% n-Octyl-β-D-glucoside detergent. Samples were sonicated in an ice-water bath at 75% amplitude for a total processing time of 3 min using a 15 s on/45 s off cycle. Lysates were clarified by centrifugation at 30,000×g for 25 min, and supernatants batch bound to 5 mL Ni-NTA resin for ≥20 min. Samples were loaded onto a gravity column and washed with 30 CV Nickel_A Buffer, then eluted in 4 CV Nickel_B Buffer (Nickel_A Buffer+250 mM imidazole) before concentrating in a 50 kDa MWCO concentrator. Samples were taken throughout the purification process and run on an SDS-PAGE protein gel, which was imaged on an imaging system in the stain-free channel following 5 min UV activation (FIG. 13A). AMBP constructs were then loaded onto an S200i 10/300 GL column and run into Nickel_A buffer (FIG. 13B). Peak fractions were pooled and concentrated in a 50 kDa MWCO concentrator. Purification of proteins expressed in the pMGBΔ vector typically yielded 25-125 nmol protein per L expression culture (FIG. 13F).

Protein Purification—pMGB Expression Vector

Proteins expressed in this vector have the following sequence architecture: 6×His-(GS)1-MBP-(GS)1-TEV-nucleoplasmin bipartite NLS-(GGGGS)3-(GS)1-MG119-X-(GGS)3-SV40 NLS (Table 5). MBP-fused constructs were purified identically to pMGBA proteins through lysis, clarification, affinity purification, and elution in Nickel_B (FIG. 13C). Following protein concentration in a 50 kDa MWCO concentrator, TEV protease added to each sample (Cf=1 UI/μL) and incubated at 4° C. overnight, gently rotating end-over-end. Samples were centrifuged (21,000×g, 4° C., 10 min) to pellet aggregates, and the supernatant was then batch-bound to 3 mL amylose resin for 30 min at 4° C., then loaded onto a gravity column. The flow-through was collected and concentrated in a 50 kDa MWCO concentrator (FIG. 13D). Again, samples were centrifuged (21,000×g, 4° C., 10 min) to pellet aggregates before loading on an S200i 10/300 GL column and run into Nickel_A buffer (FIG. 13E). Peak fractions were pooled and concentrated in a 50 kDa MWCO concentrator. Samples were taken throughout the purification process and run on an SDS-PAGE protein gel, which was imaged on an imaging system in the stain-free channel following 5 min UV activation (FIG. 13D).

A select few MG119 candidates were purified both from the pMGB and pMGBA expression vectors. A comparison of final protein yield, normalized to the initial expression culture volume, shows a trend of higher expression yields from the pMGBA vector (FIG. 13E). Purification of proteins expressed in the pMGBA vector typically yielded 2-15 nmol protein per L expression culture (FIG. 13E). Protein purification yields are shown in Table 4.

TABLE 4
Protein purification yields

| | Expression | Expression | Yield |
|---|---|---|---|
| Nuclease | Vector | Media | (nmol) |
| MG119-1 | Pmgb | TB | 8.8 |
| MG119-1 | pMGBA | TB | 105.2 |
| MG119-2 | pMGB | 2xYT | 5.0 |
| MG119-2 | pMGBA | 2xYT | 95.0 |
| MG119-3 | pMGB | 2xYT | 7.6 |
| MG119-3 | pMGBA | 2xYT | 78.1 |
| MG119-27 | pMGB | 2xYT | 11.9 |
| MG119-28 | pMGB | 2xYT | 11.6 |
| MG119-28 | pMGBA | 2xYT | 102.2 |
| MG119-32 | pMGB | 2xYT | 4.3 |
| MG119-54 | pMGB | 2xYT | 5.6 |
| MG119-64 | pMGB | 2xYT | 2.1 |
| MG119-97 | pMGB | 2xYT | 4.3 |
| MG119-109 | pMGB | 2xYT | 4.1 |
| MG119-121 | pMGB | 2xYT | 8.2 |
| MG119-128 | pMGB | 2xYT | 9.9 |
| MG119-128 | pMGBA | 2xYT | 37.0 |
| MG119-129 | pMGB | 2xYT | 2.8 |
| MG119-136 | pMGB | 2xYT | 14.3 |
| MG119-137 | pMGB | 2xYT | 10.4 |

TABLE 5
Sequence element glossary

| Element name | Element amino acid sequence |
|---|---|
| 6xHis | HHHHHH (SEQ ID NO: 1879) |
| (GS)ₙ | GS (SEQ ID NO: 1880) |
| (GGS)ₙ | GGS (SEQ ID NO: 1881) |
| (GGGGS)ₙ | GGGGS (SEQ ID NO: 1882) |
| PSP | LEVQFQGP (SEQ ID NO: 1883) |
| TEV | ENLYFQG (SEQ ID NO: 1884) |
| Nucleoplasmin | KRPAATKKAGQAKKKK (SEQ |
| bipartite NLS | ID NO: 1885) |
| SV40 NLS | PKKKRKV (SEQ ID NO: 1886) |

In Vitro Cleavage Efficiency with Purified Protein

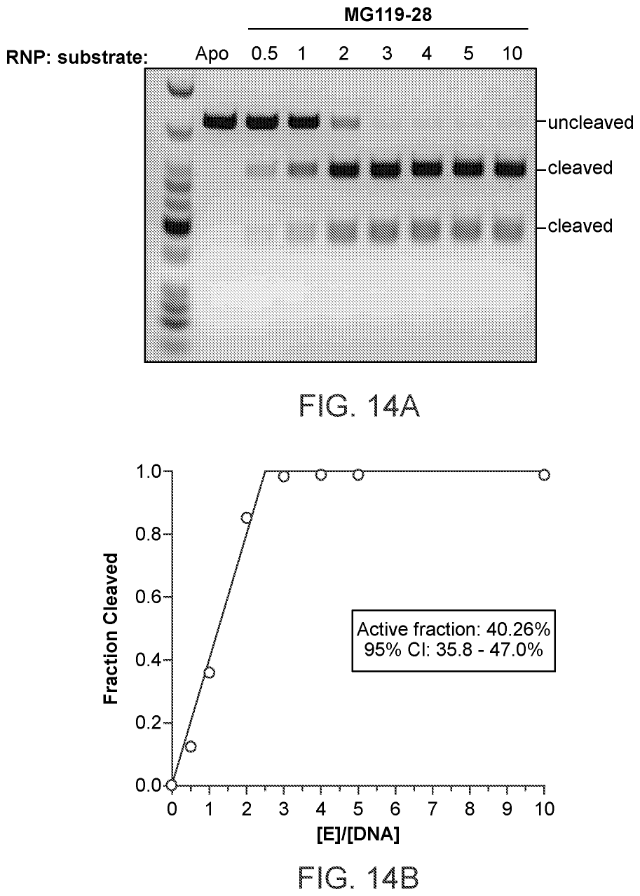

The active fraction of protein aliquots was determined in a linear DNA substrate cleavage assay. Effector proteins were preincubated with a 2-fold molar excess of sgRNA for 20 min at room temperature to form the ribonucleoprotein complex (RNP). Reactions were set up using 25 nM DNA substrate and a titration of RNP from 0.25× to 10× molar excess over substrate. The reaction buffer composition was 10 mM Tris pH 7.5, 10 mM MgCl₂, and 100 mM NaCl. The DNA substrate is 522 bp long. Successful cleavage results in fragments of 172 and 350 bp. The reaction was incubated at 37° C. for 60 min, then incubated at 75° C. for 10 min. RNase was added to each reaction (Cf=0.33 μg/μL), and samples were incubated at 37° C. for 10 min. Proteinase K was added to each reaction (Cf=60 units/mL), and samples were incubated at 55° C. for 15 min. The entirety of each reaction was then run on a 1.5% agarose gel with GelGreen dye (FIG. 14A) and imaged on an imaging system in the GelGreen channel. Percent cleaved substrate was calculated for each lane through densitometry analysis using imaging software. Active fraction was determined by the slope of the linear range of cleavage (FIG. 14B).

In Vitro Cleavage of Purified Hepa1-6 Genomic DNA with Purified Protein

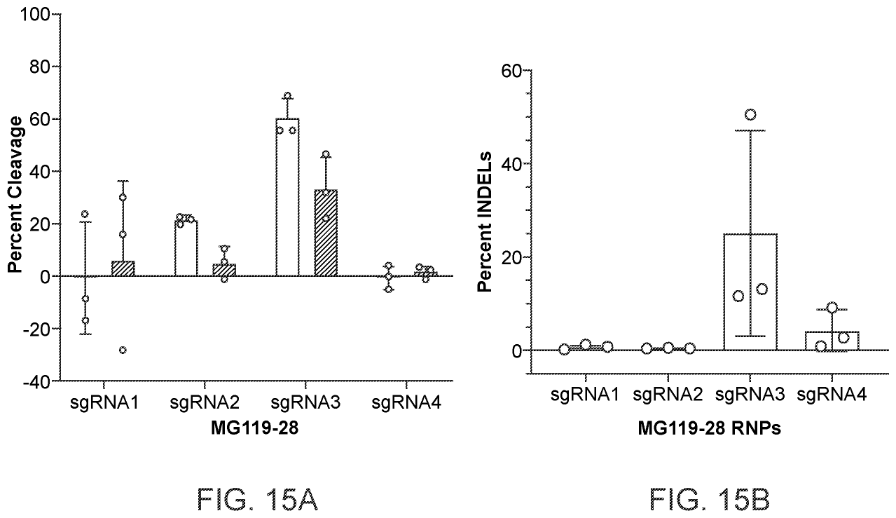

To assess cleavage of purified mouse Hepa1-6 genomic DNA (gDNA), the mouse albumin gene was targeted at intron 1 (Table 6). gDNA was extracted from Hepa1-6 cell pellets with 8 million cells using a Genomic DNA Mini purification kit and eluted in 10 mM TrisHCI at pH 8. sgRNAs were manufactured at 2 nmol then resuspended in 10 mM Tris EDTA Buffer at 20 μM (Table 6). Ribonucleoproteins (RNPs) were made by pre-incubating nucleases with targeting or non-targeting guides at a 1:2 molar ratio for 30 minutes at room temperature in 1× effector buffer (100 mM NaCl, 10 mM MgCl₂, 10 mM Tris HCl, at pH 7.5). All reactions were done in replicates of three including negative controls with no sgRNA. Following RNP formation, RNP was added to a digest reaction containing 20 ng/μL of the purified gDNA in 1× effector buffer and incubated at 37° C. for 1 hour. The nuclease was tested at two final concentrations, 7.8 and 15.6 nM. These concentrations were normalized by dividing the targeted concentrations with the active fractions for each nuclease. Following incubation, these reactions were immediately moved to 4° C., diluted 30× in water, then prepared for qPCR containing 1× Master Mix, 10 μM forward primer, 10 μM reverse primer, and 5 μM 5′-FAM and ZEN/Iowa Black fluorescence quencher Taqman probe (Table 7). A Real-Time PCR System was used with the following cycles 1) at 95° C. for 15 minutes, 2) at 95° C. for 5 seconds, and 3) at 60° C. for 1 min, where steps 2-3 were repeated 40×. Cq values were used to calculate the gDNA percent cleavage of each reaction following the Percent Cleavage Equation (below). All were normalized to the non-targeting control reactions. FIG. 15A illustrates an example of an average 60% gDNA cleavage by MG119-28 and sgRNA3 and 21% cleavage with sgRNA2 at the higher concentration of protein used.

### Percent Cleavage Equation

$$\% Cleavage = 100 - ( {2}^{- ( Cq ( experimental ) - Cq ( non - targeting control ) )} \times 100 )$$

TABLE 6
Targeting sequences in mouse albumin intron 1 and chemically modified sgRNAs

| | | Mouse Albumin Target |
|---|---|---|
| sgRNA Name | Sequence (5′-3′) | (5′-3′) |
| 119-28 sgRNA1_ | mU*mU*mG*rArArArUrArArArArUrGrArAr | AAGATTGATGAAGA |
| Mouse Alb | UrUrUrCrArArArCrCrCrCrUrUrCrGrGrGrGr | CAACTA (SEQ ID NO: |
| | GrArGrGrGrCrGrCrGrUrUrGrGrArGrCrGrCr | 1888) |
| | CrUrUrArGrUrUrUrGrArGrGrUrGrCrArGrAr | |
| | ArUrCrArArArArArArArCrUrGrCrGrArCrGr | |
| | ArUrGrGrArGrGrUrCrGrUrUrUrCrArGrUrCr | |
| | UrCrUrGrUrArCrArCrUrCrArArArArArArUr | |
| | UrCrArCrUrUrGrArGrArArArUrCrArArGrUr | |
| | GrArArUrArUrCrCrArArCrArArGrArUrUrGr | |
| | ArUrGrArArGrArCrArA*mC*mU*mA (SEQ | |
| | ID NO: 1887) | |
| 119-28 | mU*mU*mG*rArArArUrArArArArUrGrArAr | GGTCAGTGAAGAGA |
| sgRNA2_Mouse_ | UrUrUrCrArArArCrCrCrCrUrUrCrGrGrGrGr | AGAACA (SEQ ID |
| Alb | GrArGrGrGrCrGrCrGrUrUrGrGrArGrCrGrCr | NO: 1890) |
| | CrUrUrArGrUrUrUrGrArGrGrUrGrCrArGrAr | |
| | ArUrCrArArArArArArArCrUrGrCrGrArCrGr | |
| | ArUrGrGrArGrGrUrCrGrUrUrUrCrArGrUrCr | |
| | UrCrUrGrUrArCrArCrUrCrArArArArArArUr | |
| | UrCrArCrUrUrGrArGrArArArUrCrArArGrUr | |
| | GrArArUrArUrCrCrArArCrGrGrUrCrArGrUr | |
| | GrArArGrArGrArArGrA*mA*mC*mA (SEQ | |
| | ID NO: 1889) | |
| 119-28 | mU*mU*mG*rArArArUrArArArArUrGrArAr | AGTGTAGCAGAGAG |
| sgRNA3_Mouse_ | UrUrUrCrArArArCrCrCrCrUrUrCrGrGrGrGr | GAACCA (SEQ ID NO: |
| Alb | GrArGrGrGrCrGrCrGrUrUrGrGrArGrCrGrCr | 1892) |
| | CrUrUrArGrUrUrUrGrArGrGrUrGrCrArGrAr | |
| | ArUrCrArArArArArArArCrUrGrCrGrArCrGr | |
| | ArUrGrGrArGrGrUrCrGrUrUrUrCrArGrUrCr | |
| | UrCrUrGrUrArCrArCrUrCrArArArArArArUr | |
| | UrCrArCrUrUrGrArGrArArArUrCrArArGrUr | |
| | GrArArUrArUrCrCrArArCrArGrUrGrUrArGr | |
| | CrArGrArGrArGrGrArA*mC*mC*mA (SEQ | |
| | ID NO: 1891) | |
| 119-28 | mU*mU*mG*rArArArUrArAr | TCTGTGGAAACAGG |
| sgRNA4_Mouse_ | ArArUrGrArArUrUrUrCrArArArCrCrCrCrUr | GAGAGA(SEQ ID NO: |
| Alb | UrCrGrGrGrGrGrArGrGrGrCrGrCrGrUrUrGr | 1894) |
| | GrArGrCrGrCrCrUrUrArGrUrUrUrGrArGrGr | |
| | UrGrCrArGrArArUrCrArArArArArArArCrUr | |
| | GrCrGrArCrGrArUrGrGrArGrGrUrCrGrUrUr | |
| | UrCrArGrUrCrUrCrUrGrUrArCrArCrUrCrAr | |
| | ArArArArArUrUrCrArCrUrUrGrArGrArArAr | |
| | UrCrArArGrUrGrArArUrArUrCrCrArArCrUr | |
| | CrUrGrUrGrGrArArArCrArGrGrGrArG*mA* | |
| | mG*mA (SEQ ID NO: 1893) | |

TABLE 7
DNA oligos used for qPCR

| Oligo Names | Oligo sequences |
|---|---|
| 611F_HE | TGCACAGATATAAACACTTAACGGG |
| | (SEQ ID NO: 1895) |
| 869R_HE | GGGCGATCTCACTCTTGTCT |
| | (SEQ ID NO: 1896) |
| 680_HE Taqman | 5′-FAM- |
| Probe | AGCAGAGAGGAACCATTGCCACCTTCAG |
| | (SEQ ID NO: 1897) |

In Vivo Cleavage of Genomic DNA in Hepa 1-6 Cells with Purified Protein

In cell editing was demonstrated with RNP complexes of nucleases and guides targeting the mouse albumin gene at intron 1 (Table 6). Hepa1-6 cells were thawed, washed, and resuspended in Dulbecco's Modified Eagle Medium (DMEM, 10% FBS, and 1% Pen-strep). Cells were seeded at a density of 4×10⁶ cells per 15 cm dish in 30 mL of media at 37° C. After two days when the cells reached 70-80% confluency, the cells were split. Cells were trypsinized with 0.25% trypsin, then incubated at 37° C. for 30 seconds. DMEM was added then split into 3 mL and further diluted with 27 mL of media. Split cells were incubated for another two days. Prior to nucleofection, the media was aspirated from the plates, and cells were washed with 1× phosphate buffer saline pH 7.2 before trypsinizing. Trypsin was neutralized and cells were resuspended with DMEM. Cells in the cell suspension were counted to calculate the volume of cells to pellet. Each treatment downstream required a total of 100,000 cells. Cells were centrifuged at 300×g for 7 minutes, then washed in PBS pH 7.2 and resuspended in nucleofection solution.

RNP complexes were individually prepared by incubating 120 pmol of the nucleases with 120 pmol of the guides for 90 min at room temperature. 20 μL of the prepared cells were added to the RNPs. Nucleofections were performed on a Nucleofector. The nucleofected cells were transferred from the nucleofection cassettes to the 24 well plates, each well containing 500 μL of media. Following a two day incubation, gDNA from all treatments was extracted using the following cycles 1) at 65° C. for 15 min, 2) at 68° C. for 15 min, and 3) at 98° C. for 10 min and then held at 4° C. until use. The targeting window of 317 bp was amplified from the resulting extracted gDNA using the following cycles 1) at 98° C. for 10 sec, 2) at 98° C. for 1 sec, 3) at 63° C. for 5 sec, 4) at 72° C. for 15 sec, and 5) at 72° C. for 1 min repeating steps 2-5 for 30 cycles then held at 4° C. Amplicons were visualized on 2% agarose gels before cleaning and concentrating with magnetic beads with 1.8× bead volume to sample. Samples were eluted in water. INDELs were sequenced by NGS (600-cycles; Table 8) and 5% phiX for 2×301 bp paired-end reads, with a minimum of 20,000 reads per sample. INDEL analysis was performed, and results are shown in Table 9 and FIG. 15B.

TABLE 8
Oligos used for NGS PCR1

| Oligo Name | Oligo Sequence (5′-3′) |
|---|---|
| 611F_NGS | GCTCTTCCGATCTNNNNNTGCACAGATATAAACACT |
| | TAACGGG (SEQ ID NO: 1898) |
| 927R_NGS | GCTCTTCCGATCTNNNNNTTCAGCATTATAACTTAC |
| | AGGCCT (SEQ ID NO: 1899) |

TABLE 9
Percent INDELs normalized to Apo conditions

| | Replicate1 | Replicate2 | Replicate3 | Average |
|---|---|---|---|---|
| RNP | % INDEL | % INDEL | % INDEL | % INDEL |
| 119-28 sgRNA1_ | 0.70 | 0.23 | 0.87 | 0.60 |
| Mouse_Alb | | | | |
| 119-28 sgRNA2_ | 0.32 | 0.48 | 0.38 | 0.39 |
| Mouse_Alb | | | | |
| 119-28 sgRNA3_ | 50.57 | 13.12 | 11.68 | 25.12 |
| Mouse_Alb | | | | |
| 119-28 sgRNA4_ | 9.23 | 2.52 | 0.60 | 4.12 |
| Mouse_Alb | | | | |

### Example 15—Buffer Optimization for MG119 Protein Purification

Thus far, MG119 proteins have been purified in Nickel_A buffer. Nickel_A buffer is incompatible with downstream in vivo assays due to its high salinity, and rapid dilution into low-salt solutions induces protein precipitation. To optimize buffers for protein stability and downstream assay compatibility, MG119 nucleases are purified initially in high-salt buffers (750 mM NaCl) and gradually washed into a Nickel_A buffer variant with 200 mM NaCl and the zwitterionic amino acids L-arginine (50 mM) and L-glutamate (50 mM). On an empirical basis, various stabilizing sugars (ribose, sorbitol, mannitol, xylitol) are also added to the buffers to enhance protein stability in low salt buffers.

### Example 16—Fluorescence-Based Measurement of Nuclease Activity

### Cell Line Engineering

Current assays used to measure in vivo (i.e., in mammalian cell lines) nuclease activity require extensive data analysis and turnaround times of up to a week. To expedite evaluation of in vivo nuclease activity, an immortalized mammalian cell line is engineered to provide immediate data on editing of genomic DNA. K562 mammalian cells, grown in IMDM+10% FBS, are used for this assay. K562 mammalian cells are transfected with 12 pmol Cas9 protein, 60 pmol sgRNA, and 1200 ng plasmid (pUC backbone) containing an expression sequence for an mMBP-(GGS) 3-eGFP protein. Genomic integration of this construct results in constitutive expression under the synthetic MND promoter. Cells are left to grow for 6 days, passaging every 3 days. Monogenic cell lines are isolated from single cells by sorting individual GFP-expressing cells into a 96-well plate using a cell sorter.

### Fluorescence-Based In Vivo Nuclease Activity Screen

Appropriate sgRNAs are designed to direct nuclease cleavage along the mMBP and eGFP genes, such that indel formation produces a frameshift mutation resulting in loss of fluorescence. MG119 RNP complexes are formed by combining 100 pmol protein and 200 pmol sgRNA and incubating at room temperature for ≥20 min in a final volume of 5 μL. K562 cells are washed in 1×PBS and resuspended in nucleofector solution with approximately 200,000 cells per well. Cells and RNP are combined in a 96-well nucleofection plate in a final volume of 25 μL, nucleofected (K562 cells) and recovered in IMDM+10% FBS media. Cells are left to recover for 2-3 days at 37° C. To analyze, cells are washed twice with 1×PBS, then stained with 1×PBS+LIVE/DEAD dye for 20 min at room temperature. Cells are washed once more with 1×PBS before being resuspended in 1×PBS and loaded into a flow cytometer for fluorescence analysis. Positive unedited controls (nucleofected without RNP) and negative controls (non-fluorescent K562 cells) are used to establish positive and negative fluorescence gates, and cell populations are analyzed for loss-of-fluorescence in the GFP channel to evaluate in vivo nuclease activity.

### Example 17—Use for Epigenome Editing

Epigenome editing is a gene modulation technique that comprises turning genes on or off constitutively or temporarily. Such techniques may use catalytically dead Cas9 (dCas9) fused to 3 proteins: Dnmt3A, Dnmt3L, and KRAB. Dnmt3A and Dnmt3L are DNA methyltransferases. The KRAB domain mediates histone methylation. The methylation of DNA and histones in the promoter region mediates constitutive gene repression. dCas9 and a guide RNA may recruit the DNA and histone methylation complex to the promoter region, requiring no nuclease activity. Together, Dnmt3A, Dnmt3L, and KRAB are 579 aa, and dCas9 is 1,368 aa. The fusion protein consists of 1,947 aa or 5,841 nucleotides, exceeding the adeno-associated virus vector (AAV) packaging limit (4.7 Kb). Therefore, there is a need to create more compact epigenome editors. Compact Type V nucleases from the MG119 family represent great candidates for use as the dead nuclease partners in technologies for epigenome editing. Due to their small size, ranging from 350 to 700 aa, when fused to DNA and histone methylation complexes, the size of the fusion proteins may range from, for example, about 929 to about 1,279 aa, or about 2787 to about 3837 nucleotides, allowing easy packaging in AAVs.

To test MG119 fusion proteins as epigenome editors, HEK293T cells expressing GFP under a chimeric promoter (GAPDH-Srnpn) are generated by lentiviral transduction. MG119 family guide RNAs targeting the chimeric promoter are designed. Guides were manufactured, modifying the 5′ and 3′ nucleotides with 3 2′-O-methyl substituents and 3 phosphorothioate bonds for stability. Dead versions of MG119 nucleases are fused to DNA and histone methylation complexes (MG119 epigenome editors). The fusion proteins are cloned in mammalian expression plasmids under the CMV promoter. GFP expressing HEK293T cells are transfected with chemically synthesized guides and plasmids expressing MG119 epigenome editors. Transfected cells are analyzed by flow cytometry. Successful MG119 epigenome editors are determined by the loss of GFP fluorescence in transfected cells. MG119 epigenome editors are then used to target genes of therapeutic interest.

### Example 18—in Silico Identification of Mid-Size Type V Nucleases

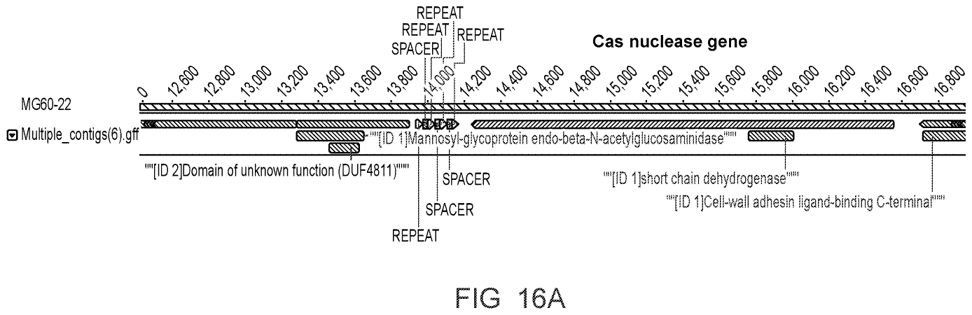

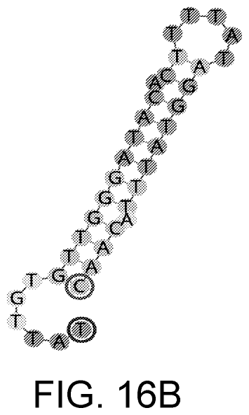

A homology search for type V Cas nucleases was performed using HMMER software. Type V nuclease sequence hits were retained if they met the following criteria: (i) the hmmsearch e-value was ≤10⁻⁵, (ii) the genes encoding the nuclease were within 1 Kb from a CRISPR array, and (iii) the amino acid sequence length ranged between 700 and 1100 aa. MMSeqs2 was used to cluster sequences at 100% amino acid identity, with coverage mode 1 and 80% coverage of the target sequence (parameters --cov-mode 1 -c 0.8 --min-seq-id 1.0). Sequence representatives were chosen to build a multiple sequence alignment using MAFFT with the Needleman-Wunsch algorithm for global alignment, and a phylogenetic tree was built. Careful examination of the phylogenetic tree led to the identification of type V nuclease family MG191 (SEQ IDs 1065-1090 and 1114-1118), and the potential CRISPR RNAs associated with them (SEQ IDs 1091-1113 and 1119-1120). A representative nuclease gene's genomic context is shown in FIG. 16A, in addition to its associated crRNA (FIG. 16B).

### Example 19—Construct Optimization for Improved MG119 Nuclease Solubility

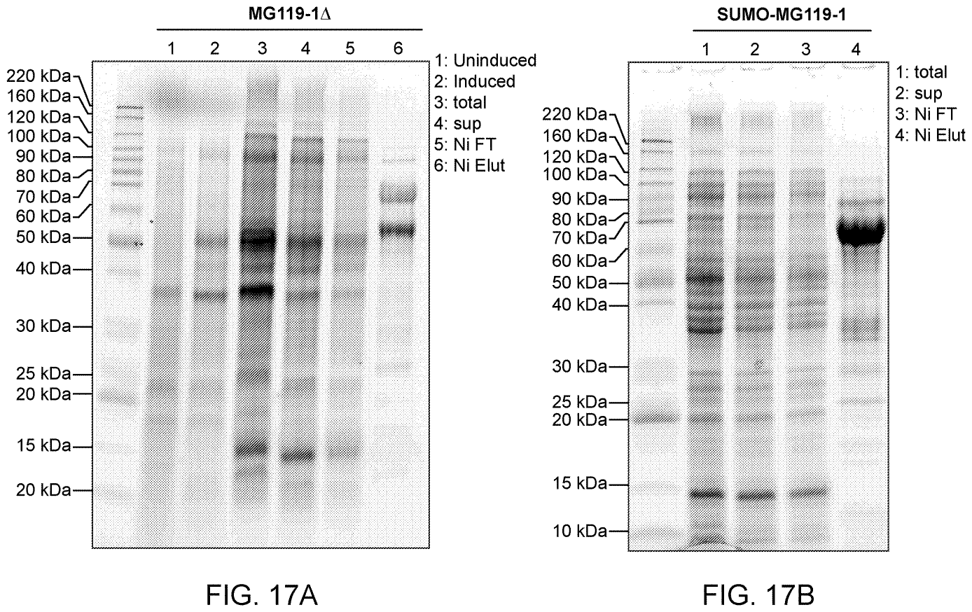

Some effector proteins purify readily with a stable fusion protein (e.g. maltose binding protein, MBP), but precipitate upon cleavage of the fusion protein using a protease. MBP is a large fusion protein (~35 kDa) and might hinder nucleofection efficiency if not removed. Therefore, poorly soluble MG119 nucleases were expressed with an N-terminal fusion to SUMO (small ubiquitin-like modifier; ~11 kDa, Tables 10 and 22), which is commonly used as an expression/solubility fusion protein. Due to its relatively small size, the SUMO domain remains fused to the effector protein, and prevents effector precipitation. Proteins with an N-terminal SUMO fusion were expressed and purified similarly to proteins expressed in the pMGBA expression vector as described in Example 14. In some cases, the inclusion of the N-terminal SUMO domain increased protein expression and solubility (FIG. 17B); by contrast, the same protein without a SUMO fusion was less pure, and was also observed readily precipitating over time (FIG. 17A). Furthermore, the SUMO-fused effector protein is also more active, as measured by an active fraction assay (e.g., MG119-1 Table 13).

TABLE 10
Amino acid sequence for SUMO domain and nucleic acids encoding same

| Protein domain | Domain amino acid sequence |
|---|---|
| SUMO | TCGGACTCAGAAGTCAATCAAGAAGCTAAGCCAGAGGTCAAGCCAGA |
| | AGTCAAGCCTGAGACTCACATCAATTTAAAGGTGTCCGATGGATCTTC |
| | AGAGATCTTCTTCAAGATCAAAAAGACCACTCCTTTAAGAAGGCTGAT |
| | GGAAGCGTTCGCTAAAAGACAGGGTAAGGAAATGGACTCCTTAAGATT |
| | CTTGTACGACGGTATTAGAATTCAAGCTGATCAGACCCCTGAAGATTT |
| | GGACATGGAGGATAACGATATTATTGAGGCTCACAGAGAACAGATTGG |
| | TGGA (SEQ ID NO: 1903) |

### Example 20—Expanded Characterization of MG119 Nucleases

### Determination of Non-Template Strand Cut Sites and Confirmation of PAM Sequences

To confirm the PAM sequences and the non-target strand cleavage site, 5 nM of nuclease amplified DNA templates and 25 nM sgRNA amplified DNA templates (including one of the spacer sequences listed in Table 11) were expressed at 37° C. for 3 hours with an in vitro protein synthesis kit. Plasmid library DNA cleavage reactions were carried out by mixing 5 nM of the target library representing all possible 8N PAMs, a 5-fold dilution of in vitro expressions, 10 nM Tris-HCl, 10 nM MgCl₂ and, 100 mM NaCl at 37° C. for 2 hours. Reactions were stopped and cleaned with PCR clean up beads and eluted in Tris EDTA pH 8.0 buffer. 3 nM of the cleavage product ends were blunted with 0.167 U/μL of Mung Bean Nuclease, 1× Mung Bean Nuclease Buffer, at 30° C. for 15-30 minutes. The ligated products were amplified by PCR with NGS primers and sequenced by NGS. Active proteins that successfully cleaved the PAM library yielded a band around 195 bp in an agarose gel electrophoresis. Bands from the agarose gels were extracted and sequenced by NGS to determine the PAM sequence.

TABLE 11
Spacer sequences for tested guides.

| Code | Sequence |
|---|---|
| U67 spacer | GTCGAGGCTTGCGACGTGGT |
| | (SEQ IDN O: 1877) |
| U40 spacer | TGGAGATATCTTGAACCTTG |
| | (SEQ ID NO: 1878) |

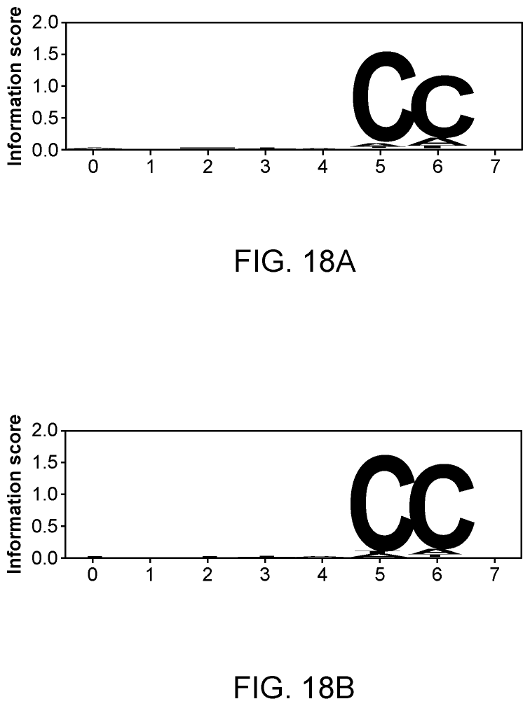

Sequence logos made using Seqlogo maker showed that the PAM sequence recognized by active effectors on the non-target strand (NTS) matches the PAM sequence on the target strand (TS). For example, for MG119-137, the PAM determined from the NTS matches the PAM obtained from the TS (FIG. 18). The cut sites on the NTS were determined from read counts at each nucleotide position. The preferred cut positions on either protospacer sequence targeted by the spacers (Table 11) are shown in Table 12, including the corresponding sgRNAs used in the experiment.

TABLE 12
MG119 nucleases preferred cut site on the non-target strand

| Nuclease | sgRNA | Non-target strand cutsite |
|---|---|---|
| MG119-1 | MG119-1_sgRNA1 | 11 & 13 |
| MG119-2 | MG119-2_sgRNA1 | 11 & 13 |
| MG119-4 | MG119-4_sgRNA1 | 11 |
| MG119-10 | MG119-10_sgRNA1 | 11 |
| MG119-19 | MG119-19_sgRNA1 | 11 |
| MG119-27 | MG119-27_sgRNA2_M2 | 11 |
| MG119-28 | MG119-28_sgRNA1 | 11 |
| MG119-32 | MG119-32_sgRNA1 | 8 & 11 |
| MG119-54 | MG119-54_sgRNA1 | 11 & 13 |
| MG119-64 | MG119-64_sgRNA1 | 13 |
| MG119-83 | MG119-83_sgRNA1 | 11 & 13 |
| MG119-121 | MG119-121_sgRNA1 | 0, 4, 9, & 11 (weak signal) |
| MG119-125 | MG119-125_sgRNA1 | 11 |
| MG119-128 | MG119-128_sgRNA2_M1 | 11 & 13 |
| MG119-129 | MG119-129_sgRNA1_M1 | 11 |
| MG119-133 | MG119-133_sgRNA1 | 11-13 |

### Spacer Length Determination

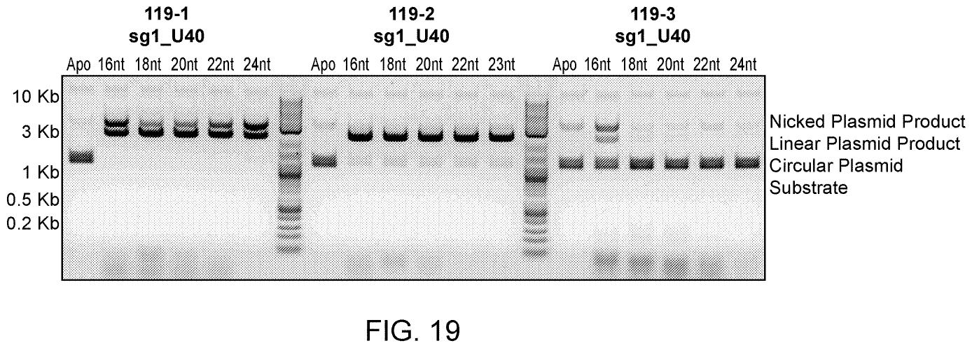

Spacer length preferences for MG119-1, MG119-2, and MG119-3 nucleases were determined by testing activity of purified enzymes with single guide RNAs (SEQ ID NOs: 432, 755, and 761) individually carrying 16 to 24 nt spacers targeting a plasmid with the preferred PAM (TTG) and protospacer sequence. 250 nM of effector was complexed with 500 nM of each guide at room temperature for 20 minutes. The RNP was then reacted with 5 nM of the target plasmid in 1× buffer at 37° C. for 1.5 hours. To analyze the cleavage products, the reactions were terminated by denaturing at 75° C. for 10 minutes. To remove remaining guide RNA, 0.1 μg/μL of RNAse A was added to each reaction and incubated for 10 minutes at 37° C. To remove the protein, 0.03 U/μL of Proteinase K was added to each reaction and incubated at 55° C. for 15 minutes. Reactions were mixed with 1× Gel Loading Dye, Purple no SDS. 6 μL were loaded in a 1% agarose gel with GelGreen dye that ran for 35 minutes at 135 V. Target plasmids were visualized using an imaging system in the GelGreen channel. Cleavage products included linearized plasmid from dsDNA breaks and nicked plasmid from ssDNA breaks. Double-stranded and single-stranded breaks were observed for MG119-1 with all spacer lengths; the 18 nt spacer guide generated fewer nicked products compared to the other spacer lengths. MG119-2 also generated double-stranded and single-stranded breaks, although minimal nicking was observed with the 18 nt spacer guide. MG119-3 seemed to favor a spacer length of 16 nt and potentially single-stranded breaks over double-stranded breaks (FIG. 19).

### Nuclease Activity Evaluation Using Purified Proteins

Not all protein purifications produce proteins that are both properly folded and catalytically active. In order to determine the quality of the purified proteins, active fraction assays were performed to quantify the percentage of purified proteins capable of cleavage. These assays were performed by titrating RNP complex relative to a constant concentration of linear DNA substrate, and measuring the DNA cleavage. Effector proteins were purified and preincubated with a 1.5- or 2-fold molar excess of sgRNA for 20 minutes at room temperature to form the ribonucleoprotein complex (RNP). Reactions were set up using 25 nM DNA substrate and a titration of RNP from 0.25× to 10× molar excess over substrate. The reaction buffer composition was 10 mM Tris pH 7.5, 10 mM MgCl₂, and 100 mM NaCl. The DNA substrate was 522 bp long. Successful cleavage resulted in fragments of 172 and 350 bp. The reaction was incubated at 37° C. for 60 min, then incubated at 75° C. for 10 min. RNase was added to each reaction (Cf=0.33 μg/μL), and samples were incubated at 37° C. for 10 min. Proteinase K was added to each reaction (Cf=60 units/mL), and samples were incubated at 55° C. for 15 min. The entirety of each reaction was then run on a 1.5% agarose gel with GelGreen dye and imaged on an imaging system in the GelGreen channel. Percent cleaved substrate was calculated for each lane through densitometry analysis using imaging software Active fraction was determined by the slope of the linear range of cleavage (Table 13).

TABLE 13
Percent active fraction and 95% confidence interval for
nucleases expressed without an N-terminal MBP fusion
protein (Δ) or with an N-terminal SUMO fusion protein

| Nuclease | Active fraction (95% CI) |
|---|---|
| MG119-1Δ | 8.00 (5.9-15.4%) |
| SUMO-MG119-1 | 14.15 (9.6-32.2%) |
| MG119-2Δ | 4.73% (4.0-6.5%) |
| MG119-3Δ | 6.76% (5.3-9.5%) |
| MG119-32Δ | 6.91% |
| MG119-54Δ | 6.79% |
| SUMO-MG119-129 | 14.47% (12.4-19.9%) |
| SUMO-MG119-136 | 16.7% |

### Example 21—Single Guide RNA Engineering

### Primary Sequence Optimization

In preparation for testing gene editing in mammalian cells, strings of four consecutive U's were replaced by single or paired mutations. These changes prevent early termination of transcription and immunogenicity in mammalian cells while preserving the secondary structure of the sgRNAs. These modified guides were designed and tested in parallel with the wild type (WT) guides using the same in vitro cleavage reactions described above. The amplified cleavage products were visualized from an agarose gel and compared to each other. The modified guides that yielded comparable PCR bands were chosen for downstream testing.

### Length Optimization for Improved Guide RNA Synthesis

Standard commercial guide synthesis quality decreases with longer RNA sequences. Therefore truncation of either wild type or optimized sgRNAs through rational design was explored, with the ultimate goal of identifying guide sequences ≤100 nt, not including the spacer sequence. Truncations were designed by predictive RNA folds and predicted RNAs were manufactured. sgRNAs were resuspended to 100 μM in TE buffer, then aliquoted to 25 μM in diH2O. Informed by active fraction measurements for each different effector, guide engineering activity tests were performed at an RNP: substrate ratio such that the WT (untruncated) sgRNA cleaves ~50% of the available substrate. All sgRNAs were tested at this same RNP: substrate ratio for a given effector. To form the RNP complex, sgRNAs were incubated with effector protein for 20 minutes at room temperature in a 1.5× molar excess over effector protein. From there, the activity test proceeded identically to active fraction measurements, starting with a 60 min incubation at 37° C. All guide variations were synthesized with the U40 spacer (Table 11). The process of designing, ordering, and testing sgRNA truncations was repeated in further rounds of engineering, and informed by data from the prior round of engineering to expand upon or design new truncations. MG119-1, MG119-3, MG119-32, MG119-54, MG119-129, and MG119-136 have undergone one round of sgRNA engineering, and MG119-2 and MG119-28 have both undergone two rounds of sgRNA engineering.

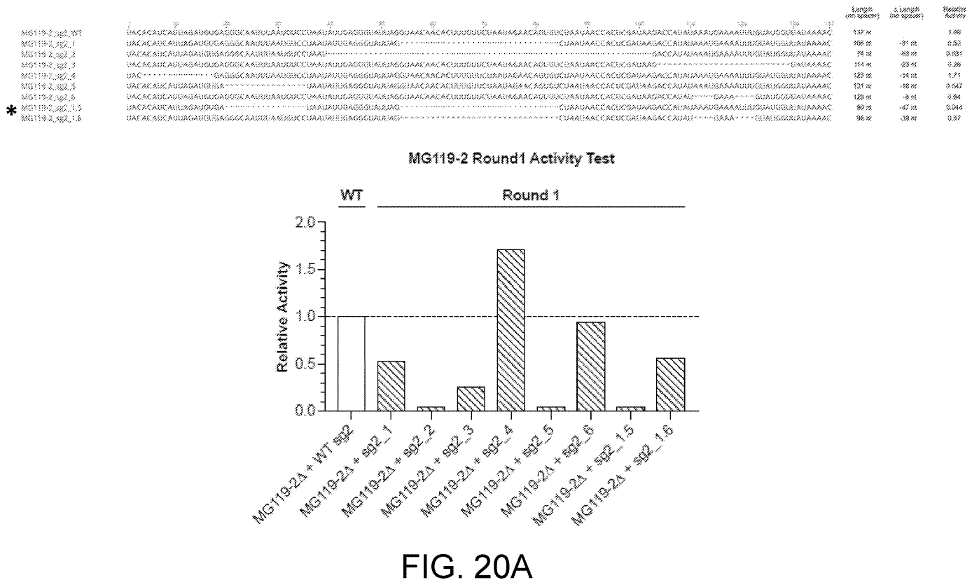

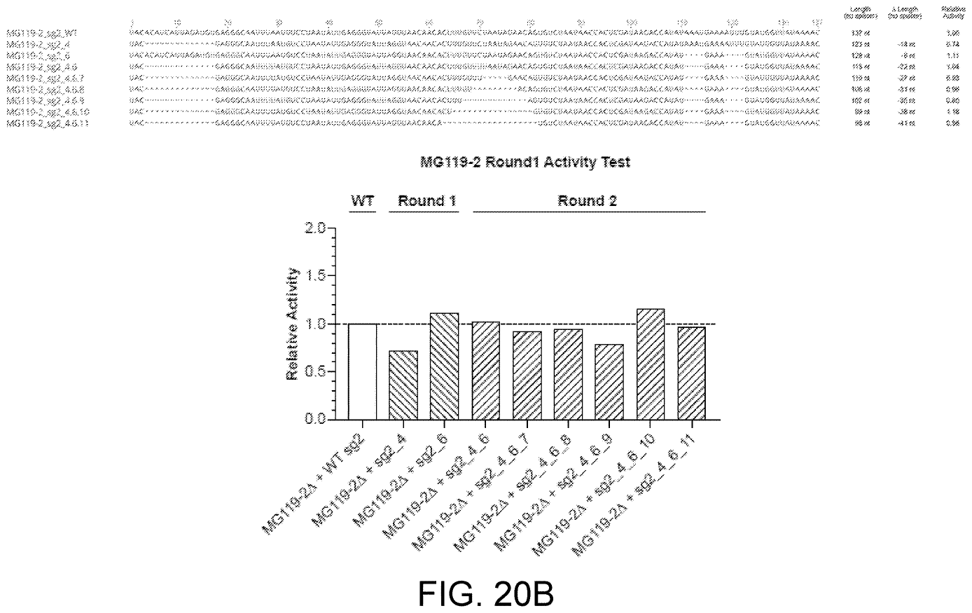

Throughout each round of guide engineering, guides that were ≥80% active relative to WT were considered of sufficient quality to be advanced. In the first round of engineering for MG119-2 guide designs, both sgRNA2_4 (124 nt; SEQ ID 659) and sgRNA2_6 (129 nt; SEQ ID 434) met this threshold, while other truncated guide scaffolds did not (FIG. 20A). During the second round of guide engineering, the sgRNA2_4 and sgRNA2_6 truncations were combined to produce the sgRNA2_4.6 guide (115 nt; SEQ ID 663), and further combined with additional truncations (FIG. 20B). All variations of MG119-2 sgRNA2 guides tested in the second round of guide engineering met the ≥80% activity threshold. The shortest guide to meet the threshold was MG119-2 sgRNA2_4.6.11 (96 nt; SEQ ID 668). Identifying guide structures that are short enough for high-yield commercial synthesis while enabling efficient enzymatic activity helps to broaden the spectrum of potential scientific and therapeutic applications for the effector proteins.

### Example 22—Mammalian Cell Editing with MG119 Nucleases

Nuclease mRNA Production

Sequences for nuclease mRNA were codon optimized for human expression, then synthesized and cloned into a high copy ampicillin plasmid. Synthesized constructs encoding T7 promoter, UTRs, nuclease ORF, and NLS sequences were digested from the backbone with HindII and BamHI, and ligated into a pUC19 plasmid backbone with T4 DNA ligase and 1× reaction buffer. The complete nuclease mRNA plasmid consisted of an origin of replication, ampicillin resistance cassette, the synthesized construct, and an encoded poly A tail. Nuclease mRNA was synthesized via in vitro transcription (IVT) using the linearized nuclease mRNA plasmid. This plasmid was linearized by incubation at 37° C. for 16 hours with SapI enzyme. The linearization reaction consisted of a 50 μL reaction containing 10 μg pDNA, 50 units Sap I, and 1× reaction buffer. Linearized plasmid was purified with Phenol: Chloroform: Isoamyl Alcohol (25:24:1, v/v), precipitated in EtOH, and resuspended in nuclease free water at an adjusted concentration of 500 ng/μL. The IVT reaction to generate nuclease mRNA was performed at 50° C. for 1 hr under the following conditions: 1 μg linearized plasmid; 5 mM ATP, CTP, GTP, and N1-methyl pseudo-UTP; 18750 U/mL Hi-T7 RNA Polymerase; 4 mM CleanCap AG; 2.5 U/mL Inorganic E. coli pyrophosphatase; 1000 U/mL murine RNase Inhibitor; and 1× transcription buffer. After 1 hr, IVT was stopped, and plasmid DNA was digested with the addition of 250 U/mL DNaseI and incubated for 10 min at 37° C. Purification of nuclease mRNA was performed. Transcript concentration was determined by UV and further analyzed by capillary gel electrophoresis.

### Cell Culture, Transfections, Next Generation Sequencing, and Indel Analysis in Mammalian Cells

Experiments with MG119-28 were performed in Hepa1-6 cells grown and passaged in in Dulbecco's Modified Eagle's Medium plus 1×NEAA supplemented with 10% (v/v) fetal bovine serum and 1% Pen/Strep at 37° C. with 5% CO₂. Experiments with MG119-2, MG119-125, and MG119-129 were performed in K562 cells grown and passaged in Dulbecco's Modified Eagle's Medium supplemented with 10% (v/v) fetal bovine serum at 37° C. with 5% CO₂.

For experiments where the nuclease was delivered as mRNA, 500 ng mRNA and 150 pmol of sgRNA were mixed together and incubated on ice until cells were prepared. For experiments where the nuclease and guide were delivered as an RNP complex, 100 pmol of purified nuclease was incubated with 200 pmol of sgRNA at room temperature for approximately 30 minutes prior to transfection. The sgRNA sequences used with MG119-28 targeting mouse Albumin intron 1 are SEQ ID NOs: 625-628. The sgRNA sequences used with MG119-2 targeting human TRAC exon 3 are SEQ ID NOs: 767-798. The sgRNA sequences used with MG119-129 targeting human APOA1 exon 3 are SEQ ID NOs: 979-1021. The sgRNA sequences used with MG119-125 targeting human APOA1 exon 3 are SEQ ID NOs: 831-904.

Approximately 1×10⁵ cells were transfected with the RNP complex or mRNA plus sgRNA in a nucleofector. Transfected cells were grown for 3 days, harvested, and gDNA was extracted. Targeted regions for indels were amplified using Q5 High-Fidelity DNA polymerase with primers and extracted DNA as the templates and PCR products were purified. PCR primers appropriate for use in NGS-based DNA sequencing were generated, optimized, and used to amplify the individual target sequences for each guide RNA. The amplicons were sequenced and analyzed with a proprietary Python script to measure indel frequency.

The percentage of amplicons from NGS amplicon sequencing that contained insertions or deletions obtained with MG119-28 guides targeting intron 1 of the mouse Albumin gene are shown in Table 14. Compared to previous results with MG119-28 using RNP delivery, mRNA delivery boosted editing at mouse Albumin up to >90% at one target site (mALB sgRNA3) and 43% at another (mALB sgRNA4).

TABLE 14
MG119-28 Hepa1-6 percent indels

| | mRNA Rep1 | mRNA Rep2 |
|---|---|---|
| Guide | % INDEL | % INDEL |
| mALB sgRNA1 | 3 | 4 |
| mALB sgRNA2 | 0.8 | 1 |
| mALB sgRNA3 | 91 | 92 |
| mALB sgRNA4 | 44 | 42 |

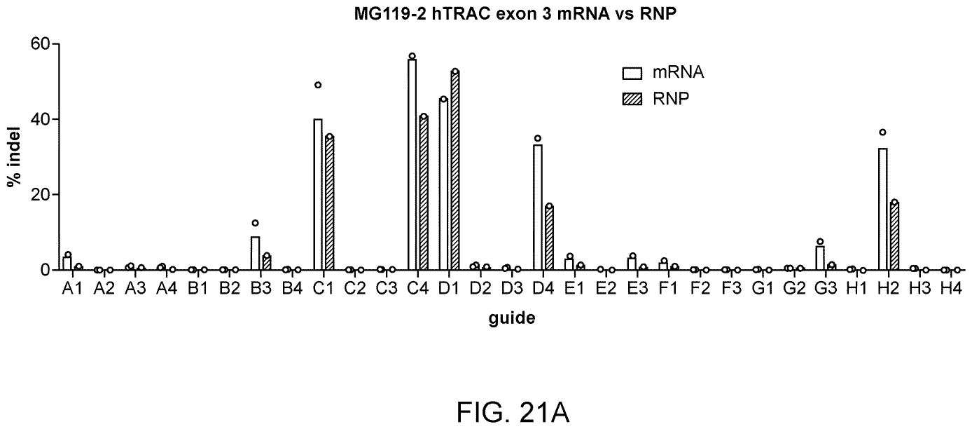

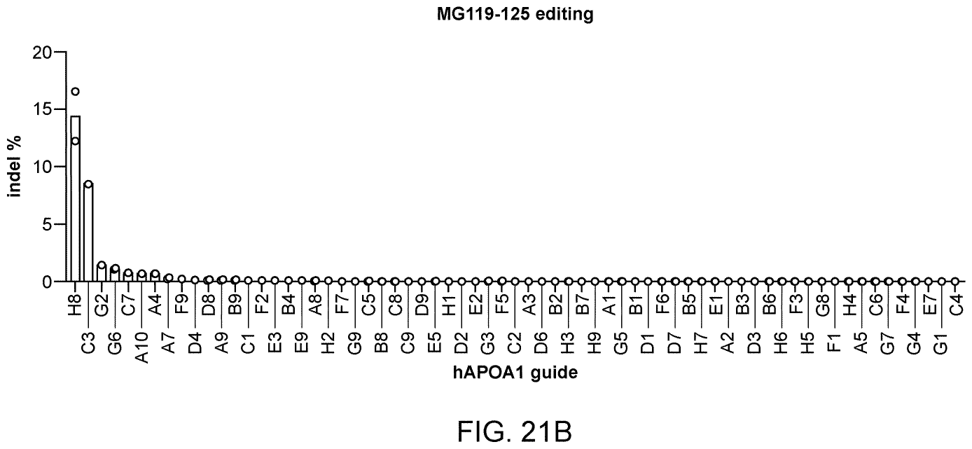

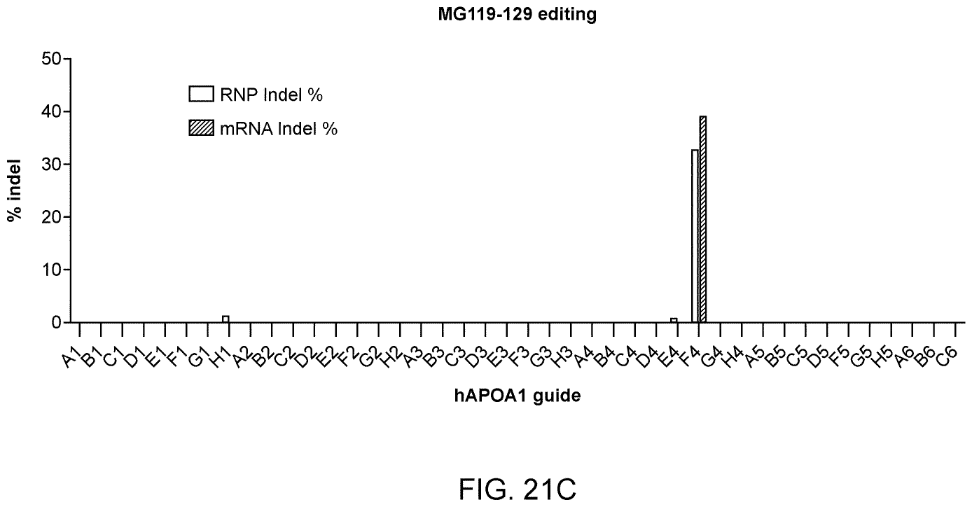

The percentage of amplicons from NGS amplicon sequencing that contained insertions or deletions obtained with MG119-2 guides targeting exon 3 of the human TRAC gene are shown in FIG. 21A. Five target sites in TRAC displayed >10% editing by MG119-2 with delivery by both mRNA and RNP, with the highest editing at 57%.

MG119-125 was delivered as mRNA to K562 cells targeting human APOA1 exon 3. The percentage of indel amplicons from NGS amplicon sequencing are shown in FIG. 21B. Four target sites displayed detectable editing, with one target seeing up to 17% editing.

MG119-129 was delivered as both mRNA and RNP to K562 cells targeting human APOA1 exon 3. The percentage of indel amplicons from NGS amplicon sequencing are shown in FIG. 21C. One target site displayed 33% editing using RNP delivery and 39% editing using mRNA delivery.

### Example 23—Mammalian Cell Editing with MG119 Nucleases

Nuclease mRNA Production

Sequences for MG119 nucleases mRNA were codon optimized for human expression then synthesized and cloned into a high copy ampicillin plasmid. Synthesized constructs encoding T7 promoter, UTRs, nuclease ORF, and NLS sequences were digested from the backbone with HindII and BamHI enzymes, and ligated into a pUC19 plasmid backbone with T4 DNA ligase and 1× reaction buffer. The complete nuclease mRNA plasmid consists of an origin of replication, ampicillin resistance cassette, the synthesized construct, and an encoded poly A tail. Nuclease mRNA was synthesized via in vitro transcription (IVT) using the linearized nuclease mRNA plasmid. This plasmid was linearized by incubation at 37° C. for 16 hours with SapI enzyme. The linearization reaction consisted of a 50 μL reaction containing 10 μg pDNA, 50 units Sap I, and 1× reaction buffer. Linearized plasmid was purified with Phenol:Chloroform:Isoamyl Alcohol (25:24:1, v/v), precipitated in EtOH, and resuspended in nuclease-free water at an adjusted concentration of 500 ng/μL. The IVT reaction to generate nuclease mRNA was performed at 50° C. for 1 hour under the following conditions: 1 μg linearized plasmid; 5 mM ATP, CTP, GTP, and Nl-methyl pseudo-UTP; 18750 U/mL Hi-T7 RNA Polymerase; 4 mM CleanCap AG; 2.5 U/mL Inorganic E. coli pyrophosphatase; 1000 U/mL murine RNase Inhibitor; and 1× transcription buffer. After 1 hour, IVT was stopped, and plasmid DNA was digested with the addition of 250 U/mL DNaseI and incubated for 10 min at 37° C. Nuclease mRNA was purified. Transcript concentration was determined by UV and further analyzed by capillary gel electrophoresis on a Fragment Analyzer.

### Cell Culture, Transfections, Next Generation Sequencing, and Indel Analysis in Mammalian Cells

Experiments were performed in K562 cells grown and passaged in Iscove's Modified Dulbecco's Medium supplemented with 10% (v/v) fetal bovine serum at 37° C. with 5% CO₂.

For nucleofection with mRNA (MG119-2 and MG119-28), 500 ng mRNA and 200 pmol of sgRNA were mixed together and incubated on ice until cells were prepared. For nucleofection with RNP (MG119-32), 100 pmol of nuclease was complexed with 200 pmol of sgRNA for 30 minutes at room temperature and then placed on ice until cells were prepared. The sgRNA sequences used with MG119-2, MG119-28, and MG119-32 targeting human Albumin intron 1, human APOA1 exon 3, and AAVS1 are SEQ ID NOs: 1121-1169, 1219-1237, 1257-1324, 1393-1441, 1491-1506, 1523-1562, 1603-1632, 1663-1669, and 1677-1686.

Approximately 1×10⁵ cells were transfected with mRNA plus sgRNA. Transfected cells were grown for 3 days, harvested, and gDNA was extracted. Targeted regions for indels were amplified using a high fidelity DNA polymerase with primers and extracted DNA as the templates. PCR products were purified by. PCR primers appropriate for use in NGS-based DNA sequencing were generated, optimized, and used to amplify the individual target sequences for each guide RNA. The amplicons were sequenced through next generation sequencing and analyzed with a proprietary Python script to measure indel frequency.

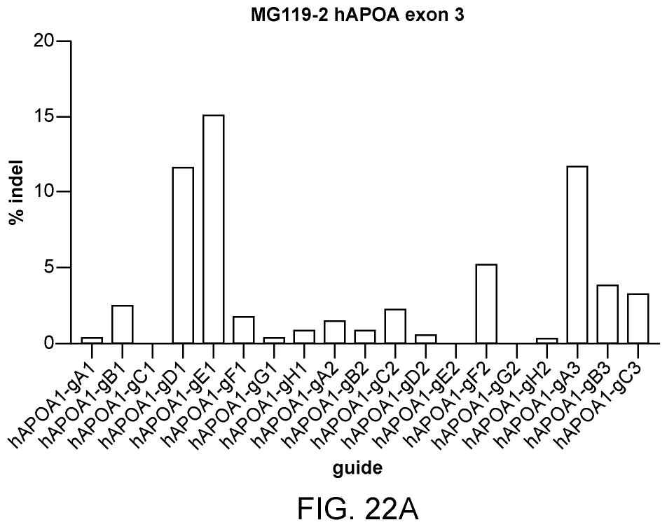

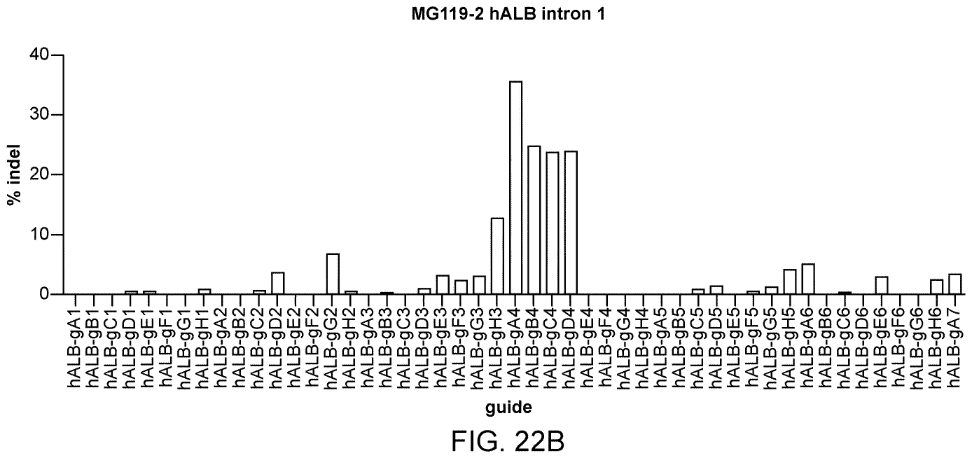

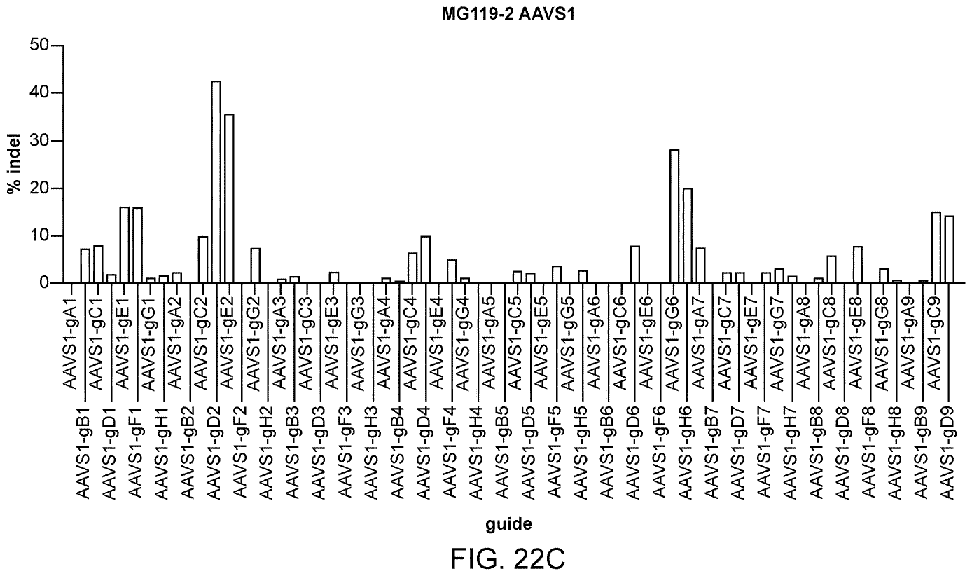

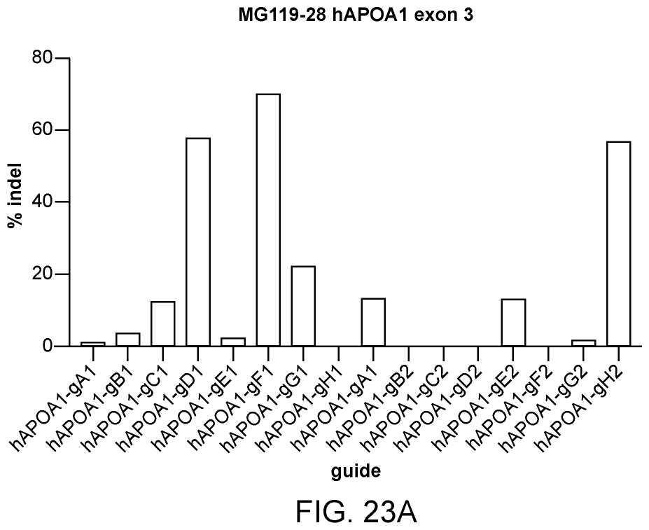

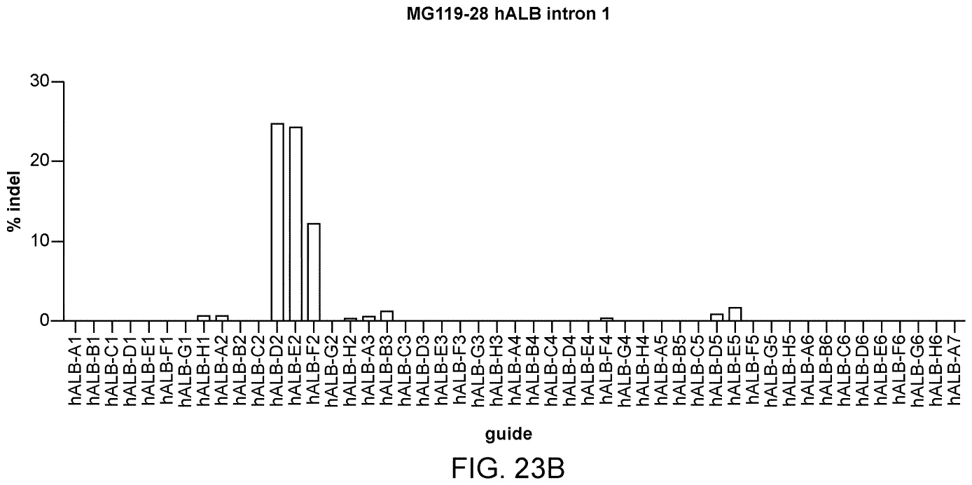

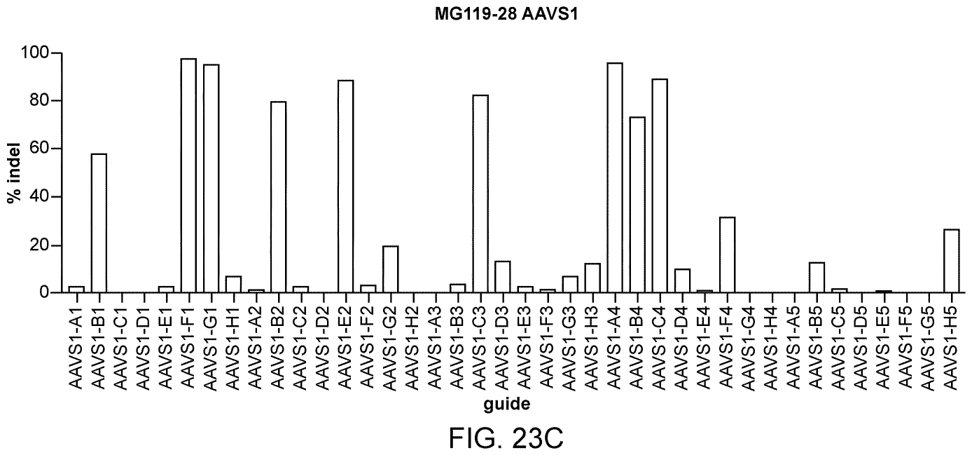

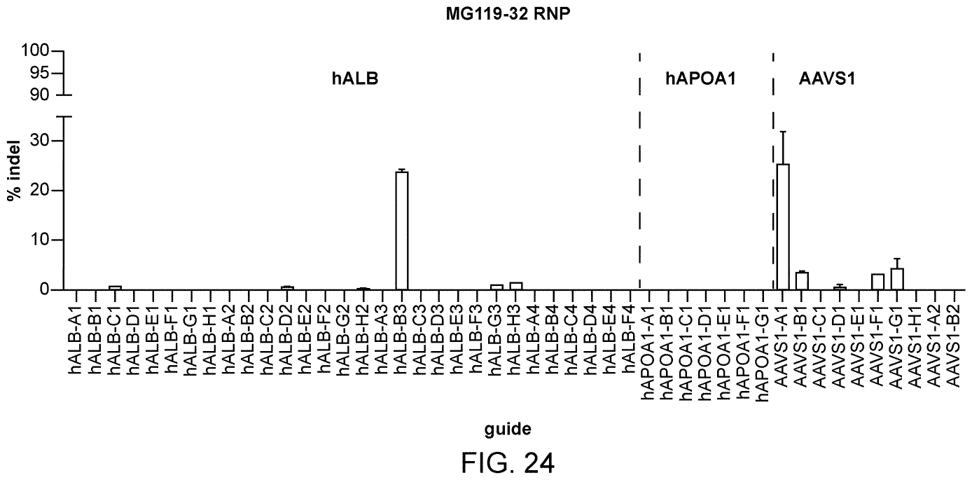

The percentage of amplicons from NGS amplicon sequencing that contain insertions or deletions obtained with MG119-2 guides targeting intron 1 of the human ALB gene, exon 3 of the human APOA1 gene, and AAVS1 are shown in FIG. 22. Sixteen target sites across all three loci displayed >10% editing, with the highest editing at 43% for AAVS1-gD2. The percentage of amplicons from NGS amplicon sequencing that contain insertions or deletions obtained with MG119-28 guides targeting intron 1 of the human ALB gene, exon 3 of the human APOA1 gene, and AAVS1 are shown in FIG. 23. Twenty-five target sites across all three loci displayed >10% editing, twelve of which were >50%. At AAVS1, two target sites achieved >90% editing. The percentage of amplicons from NGS amplicon sequencing that contain insertions or deletions obtained with MG119-32 guides targeting intron 1 of the human albumin (ALB) gene, exon 3 of the human APOA1 gene, and AAVS1 are shown in FIG. 24. Two target sites achieved >20% editing.

### Example 24—Guide RNA Engineering for the MG119 Family

### Split Guide Engineering

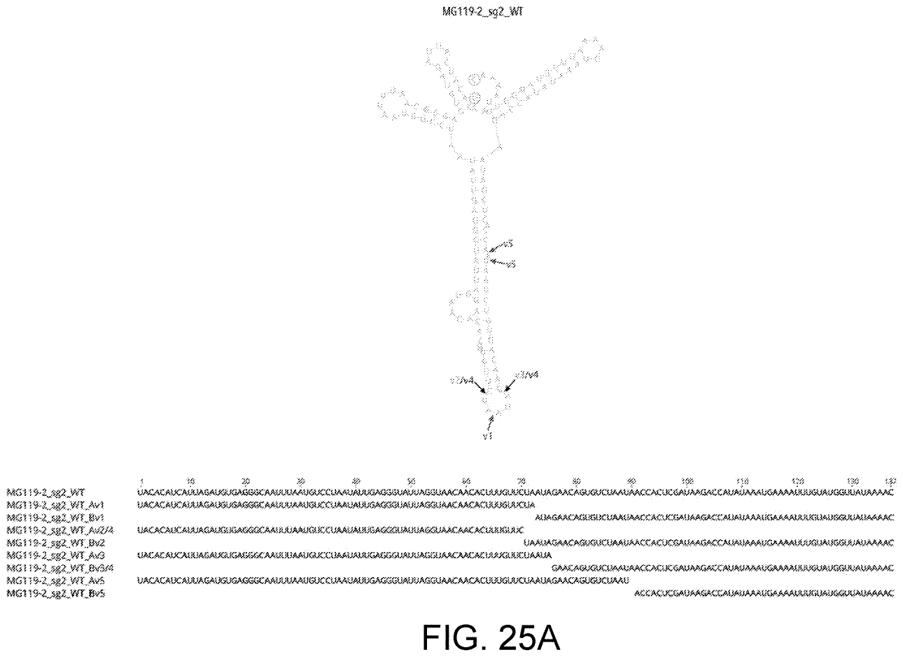

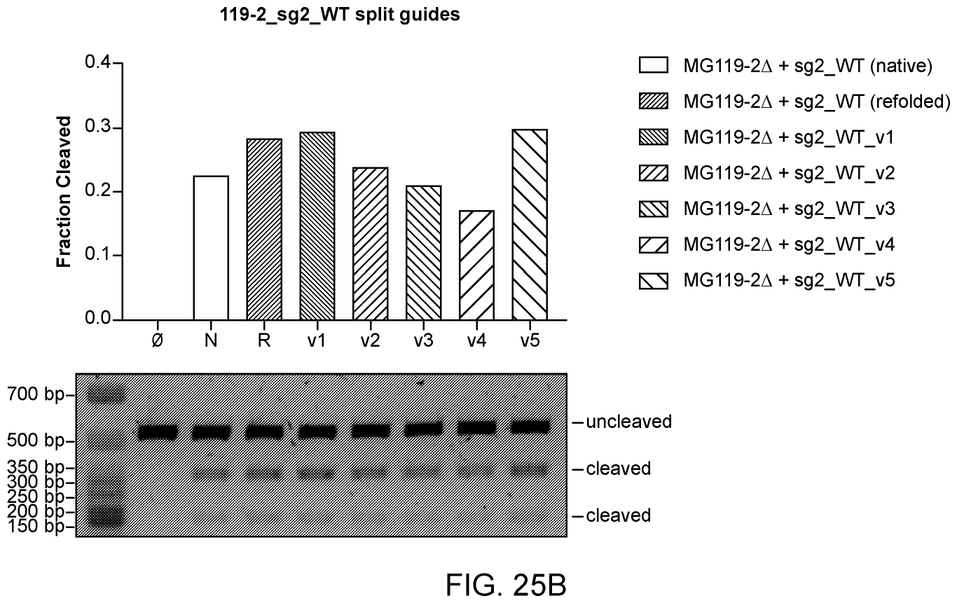

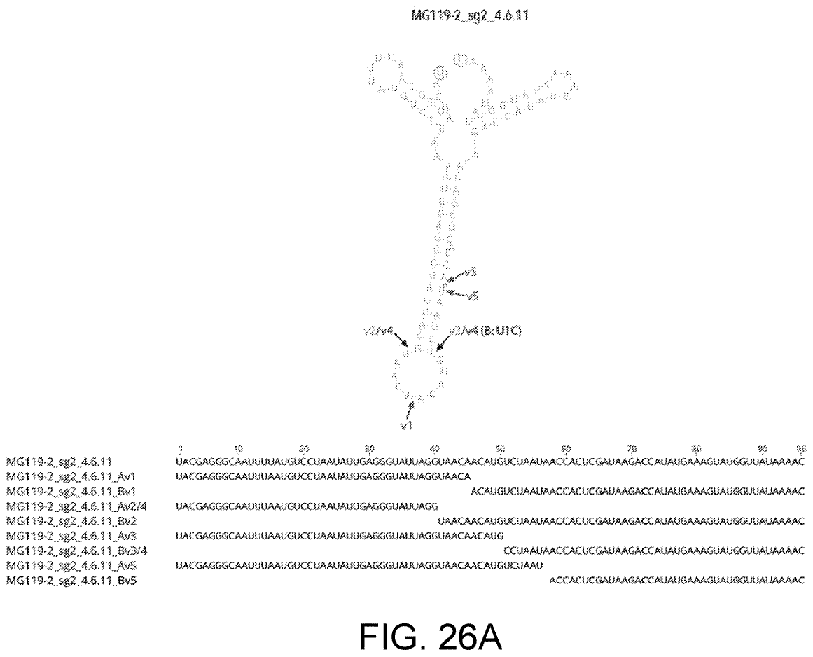

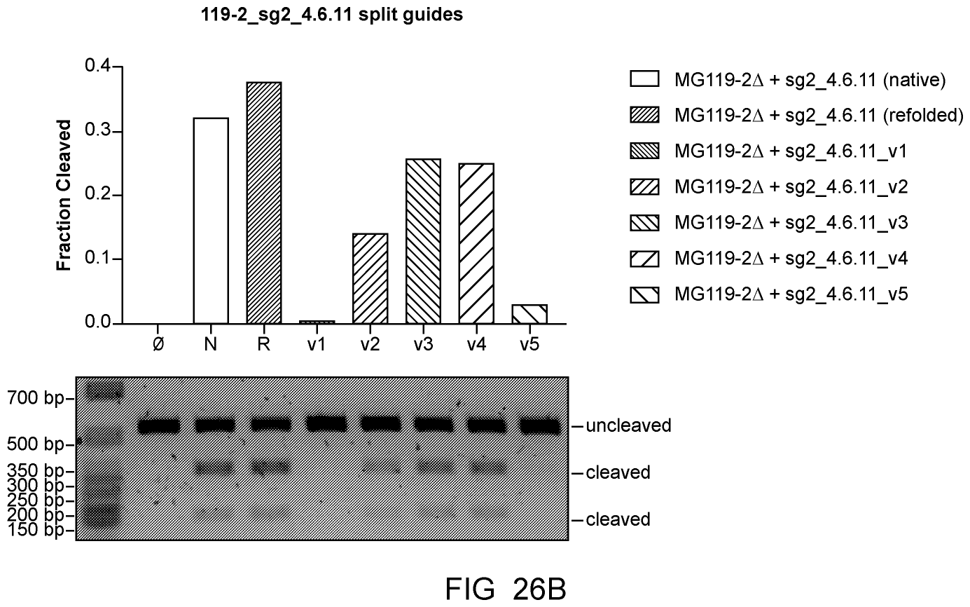

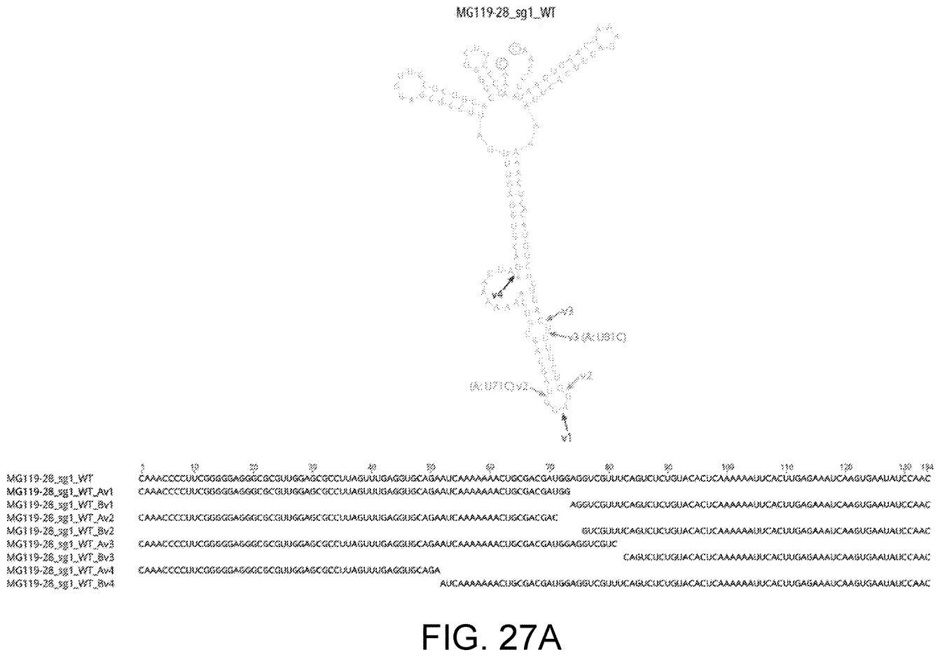

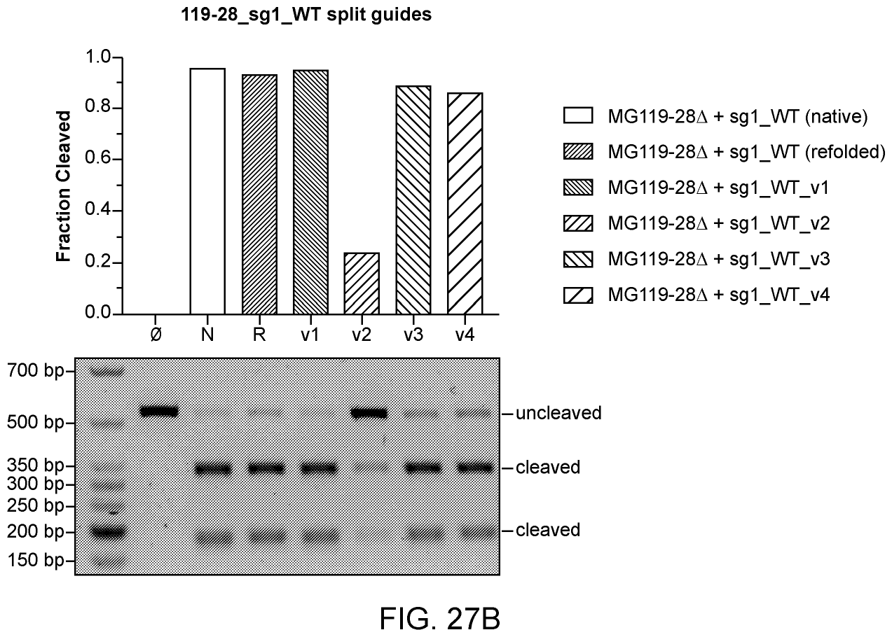

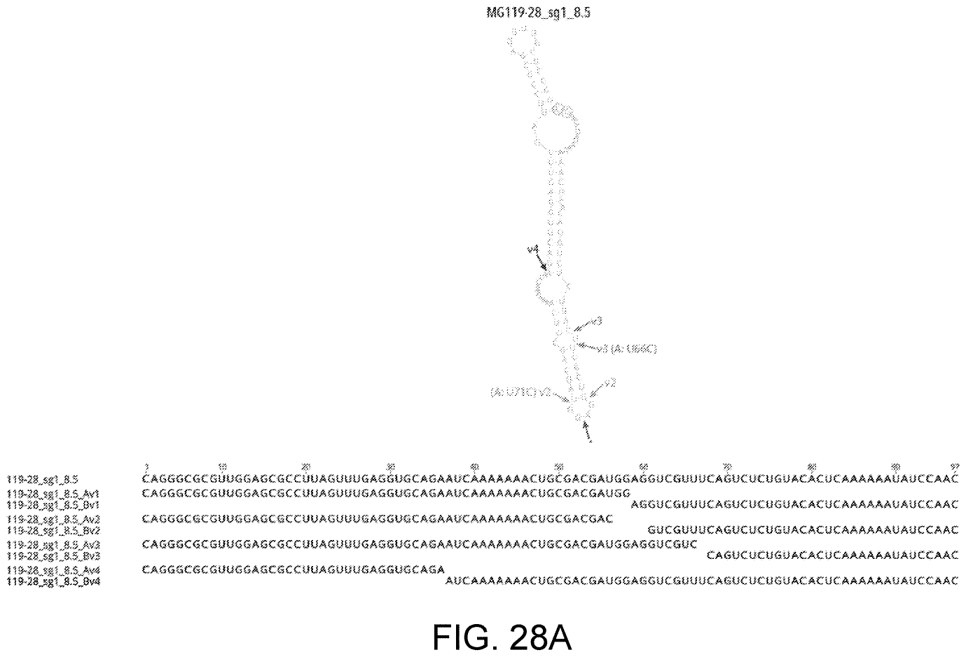

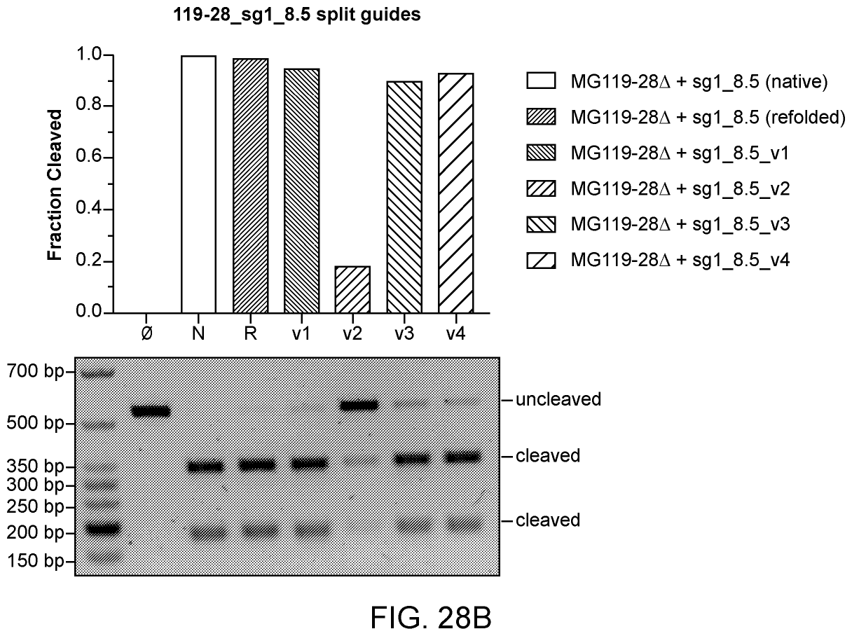

Most MG119 effectors have single-guide scaffolds ranging from 130-160 nt (without spacer). While guide minimization via truncation of unnecessary regions is advantageous, this may not always be possible. A viable alternative may be to split the guide RNA into two halves, which are annealed prior to RNP complex formation. To test this idea, four or five split guide designs each were evaluated for MG119-2_sg2_WT (FIG. 25A), MG119-2_sg2_4.6.11 (FIG. 26A), MG119-28_sg1_WT (FIG. 27A), and MG119-28_sg1_8.5 (FIG. 28A). Prior to RNP complexing, the two halves of each split guide were warmed to 80° C. in an unfolding buffer (20 mM HEPES pH 7.0, 100 mM NaCl, 0.1 mM EDTA) for 2 min. As a control, the parent sgRNA for each was also unfolded and re-annealed alongside the split guides. Subsequently, an equal volume of pre-warmed (80° C.) 2× annealing buffer (40 mM HEPES pH 7.0, 200 mM NaCl, 2 mM MgCl₂) was added to the guides, and the reaction was slow-cooled to 4° C. at 0.1° C./s. From this point on, in vitro cleavage reactions were performed as previously described in Example 20. These assays included both a non-refolded parent sgRNA (N, Native) and a refolded parent sgRNA (R, Refolded). For MG119-2, reactions were set up with 25 nM DNA substrate, 50 nM MG119-2 effector protein, and 75 nM guide RNA. For MG119-28, reactions were set up with 20 nM DNA substrate, 400 nM MG119-28 effector protein, and 600 nM guide RNA.

For MG119-2_sg2 (SEQ ID NO: 656), all split guide designs (SEQ ID NOs: 1697-1704) were active to varying degrees (FIG. 25B). For MG119-2_sg2_4.6.11 (SEQ ID NO: 668), split guides v1 and v5 were virtually inactive, and v2 split guides were only about half as active as the parent sgRNA (FIG. 26B; SEQ ID NOs: 1705-1712). For both MG119-28_sg1 (SEQ ID NO: 686; FIG. 27B) and MG119-28_sg1_8.5 (SEQ ID NO: 704; FIG. 28B), all versions of split guides were highly active, with the exception of v2 (SEQ ID NOs: 1713-1720 and 1721-1728, respectively). These data suggest that split guide engineering is feasible for systems with large guides, and that multiple split points can be active.

### Single Guide RNA Minimization for Improved Synthesis

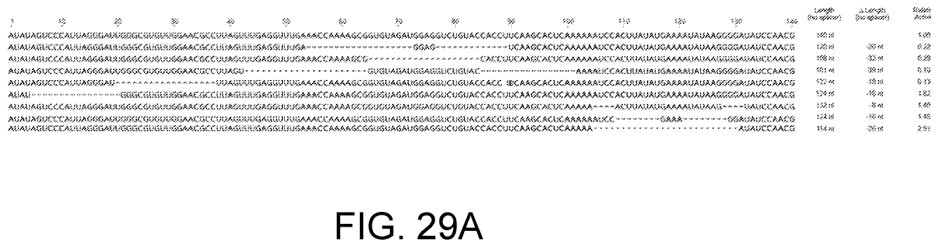

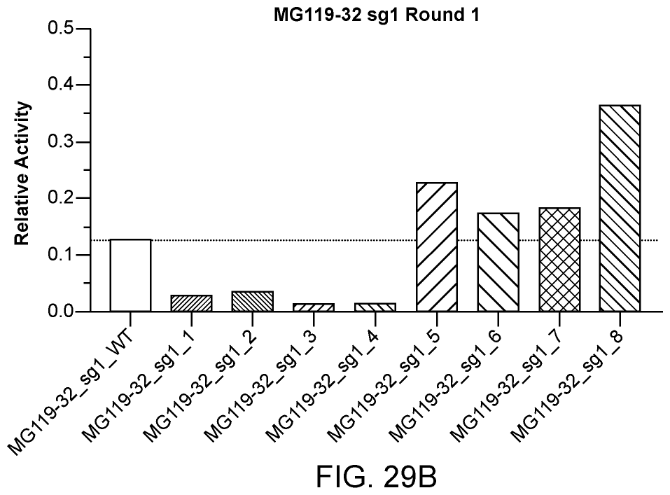

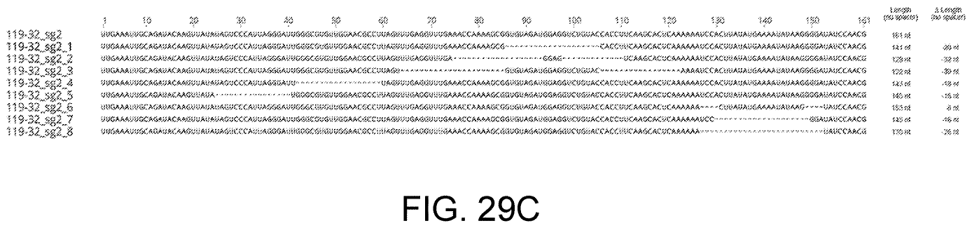

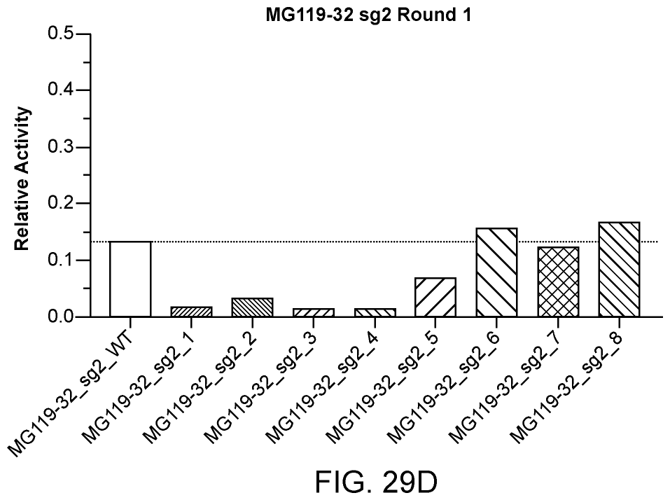

Length optimization for the sgRNA of the MG119-32 effector was performed similarly to methods described in Example 21. Two WT variants of sgRNA were initially tested for MG119-32: sg1 (SEQ ID NO: 448) and sg2 (SEQ ID NO: 720). Guide trimming was performed on both the sg1 and sg2 WT guides. For the first round of guide engineering, similar truncations were designed for both sg1 (FIG. 29A; SEQ ID NOs: 712-719) and sg2 (FIG. 29C; SEQ ID NOs: 721-728). Cleavage tests were performed similarly to methods described in Example 20, using 20 nM substrate, 200 nM MG119-32 effector protein, and 300 nM sgRNA.

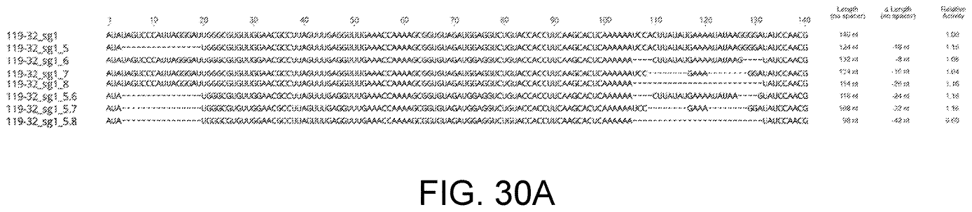

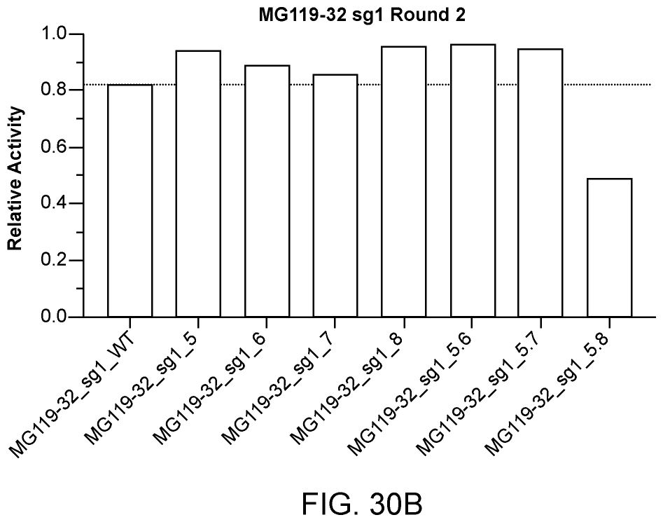

A similar trend was seen for both panels of sgRNAs, in that only truncations 5, 6, 7, and 8 showed any notable activity relative to the WT sgRNA (FIGS. 29B and 29D). Percent cleaved substrate was calculated for each lane through densitometry analysis. Since cleavage appeared overall higher in the sg1 sgRNA panel, only sg1 was advanced to round 2 of guide engineering. For the second round of guide engineering, truncation 5 was combined with truncation 6 (MG119-32_sg1_5.6; SEQ ID NO: 1729), truncation 7 (MG119-32_sg1_5.7; SEQ ID NO: 1730), or truncation 8 (MG119-32_sg1_5.8; SEQ ID NO: 1731) to make three different double-truncations (FIG. 30A). Cleavage tests were performed similarly to the first round of guide engineering, using 20 nM substrate, 500 nM MG119-32 effector protein, and 750 nM sgRNA.

Among the new double-truncation guides, 119-32_sg1_5.6 and 119-32_sg1_5.7 cleaved with high activity comparable to the WT sg1 sgRNA. Guides that contribute to at least 80% cleavage relative to WT are considered successful and are used as a starting point for the second round of guide engineering. By contrast, 119-32_sg1_5.8 had impaired activity relative to the WT sg1 sgRNA. These data suggest that the large sgRNAs of MG119 family effector proteins are amenable to guide trimming without significant loss of activity.

Trimmed sgRNA Characterization

During guide length optimization, guide scaffold truncations are primarily advanced through engineering rounds based on dsDNA cleavage activity. It is possible, however, that guide length optimization can inadvertently change tangential enzymatic activities, such as nicking, collateral ssDNA cleavage, and collateral ssRNA cleavage.

To determine if guide engineering of the MG119-2 and MG119-28 guides changed secondary nickase activity, a cleavage assay was performed using circular plasmid DNA as the substrate. In this assay, uncleaved substrate remains supercoiled. Fully-cleaved substrate becomes linear and migration on an agarose gel therefore slows relative to the supercoiled uncut reactant. Furthermore, nicked plasmid does not become linearized, but its supercoil relaxes; this species migrates even slower than the fully-cleaved linear product on an agarose gel, and is thus identifiable. Tracking the intensity of this relaxed product is therefore a readout of the prevalence of nicking activity. Furthermore, performing this reaction with the WT guide, the fully-trimmed guide, and each variation that contributed to the fully-trimmed guide will provide insight into what portions of the guide may mediate such activity (if any differences are observed).

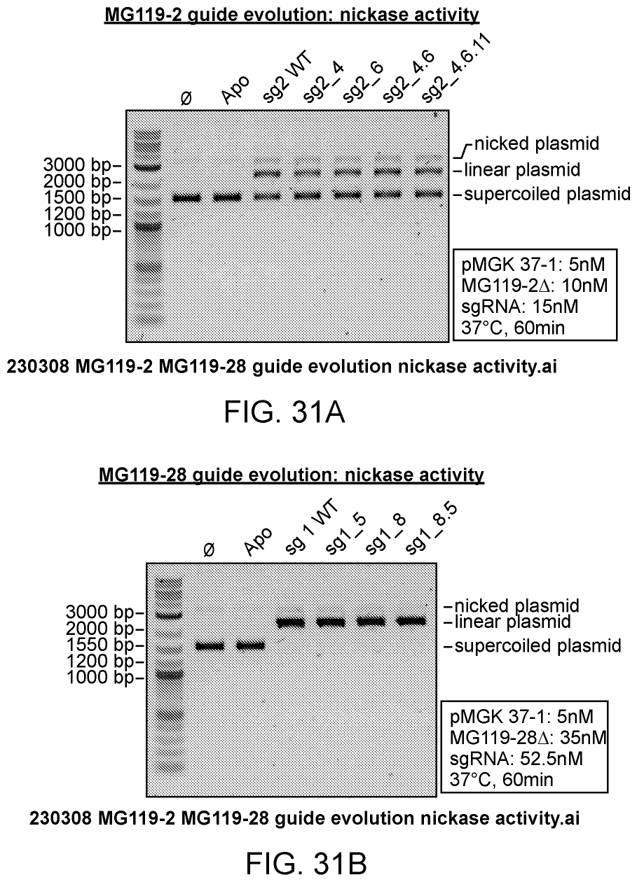

MG119-2 cleavage reactions were performed as described in Example 20, using 5 nM plasmid substrate, 10 nM MG119-2 effector protein, and 15 nM sgRNA. The data suggest that nicking activity remains largely unchanged for all guides tested (FIG. 31A). MG119-28 cleavage reactions were performed as described in Example 20, using 5 nM plasmid substrate, 35 nM MG119-28 effector protein, and 52.5 nM sgRNA. The data suggest that nicking activity remains largely unchanged for all guides tested (FIG. 31B).

### Example 25—Mid-Size Type V Nucleases Discovery and In Vitro Activity Assay

### In Silico Discovery of Novel Mid-Size Type V Nucleases

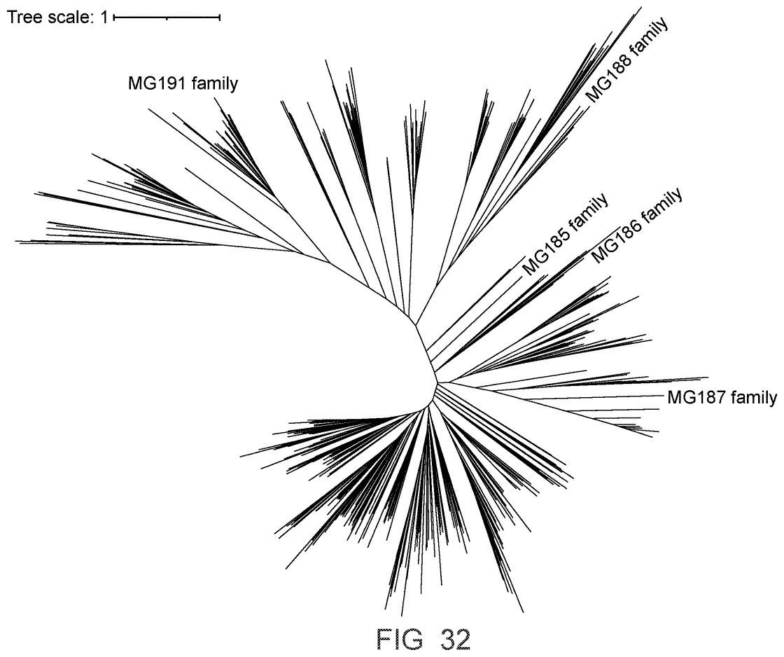

The MG191 family of nucleases (750-800 aa) was identified with crRNAs encoded next to them. Similar methods led to the discovery of four other families (MG185, MG186, MG187, and MG188; SEQ ID NOs: 1746-1752) of type V nucleases between 700 and 1100 aa long. In brief, Cas12 hmm hits with an e-value≤10-5 that were within 1 Kb from a CRISPR array and 700-1100 aa long were clustered at 100% amino acid identity (--cov-mode 1 -c 0.8 --min-seq-id 1.0). Representative sequences were aligned using MAFFT with the Needleman-Wunsch algorithm for global alignment, and a phylogenetic tree was built using FastTree (FIG. 32).

### MG191 DNA Templates for In Vitro Transcription and Translation

Minimal arrays (SEQ ID NOs: 1732-1738) were designed to include a T7 promoter, predicted repeat, a U40 spacer sequence (TGGAGATATCTTGAACCTTGCATCCCCGGA, SEQ ID NO:1901), a second identical repeat, followed by a primer binding sequence, in that order. In a second design, the repeat orientations in the minimal arrays were reversed while keeping the spacers constant to test which orientation of the repeats was active. The reverse complement of these sequences were ordered as single stranded ultramers to be annealed with a complementary T7 promoter oligo for transcription. Transcription templates were prepared by mixing and incubating 20 μM of T7 promoter oligo, 20 μM of the reverse complement minimal arrays, and 0.7×IDT duplex buffer then annealing by heating to 95° C. for 2 minutes then cooling to room temperature at 0.1° C./sec.

E. coli codon-optimized nuclease plasmids were generated with a pTAC driven expression vector flanked by NLS sequences at each termini and an N-terminal His tag and MBP with prescission protease site (pMGD expression vector as described in Example 14). Linear nuclease templates for in vitro transcription/translation (IVTT) were amplified by PCR, which simultaneously added a T7 promoter for expression, then cleaned and eluted in 10 mM Tris HCl pH 8.0. PCR templates were verified for yield and purity.

### MG191 Transcription of Minimal Array (Pre-crRNA)

dsDNA minimal array templates described above were used for synthesis of pre-crRNA.

RNA was synthesized with and cleaned. Transcription products were verified for yield and purity. RNA generated here was used to test for activity of purified protein.

### MG191 In Vitro Cleavage Assay and PAM Sequence Determination

As previously described, 5 nM of nuclease amplified DNA templates and 25 nM of promoter-annealed minimal array DNA templates (including the U40 spacer sequence TGGAGATATCTTGAACCTTGCATCCCCGGA (SEQ ID NO: 1901)) were expressed at 37° C. for 2 hours with an in vitro protein expression system. Plasmid library DNA cleavage reactions were carried out by mixing 5 nM of the target library representing all possible 8N PAMs with a 5-fold dilution of in vitro expressions in 10 mM Tris-HCl pH 7.9, 10 mM MgCl₂, 100 μg/mL BSA, and 50 mM NaCl at 37° C. for 1 hour. Reactions were stopped and cleaned with using PCR clean up beads and eluted in Tris EDTA pH 8.0 buffer. 3 nM of the cleavage product ends were blunted with 3.33 μM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, 20 U/μL T4 DNA ligase at room temperature for 20 minutes. The ligated products were amplified by PCR with NGS primers and sequenced by NGS to obtain the PAM.

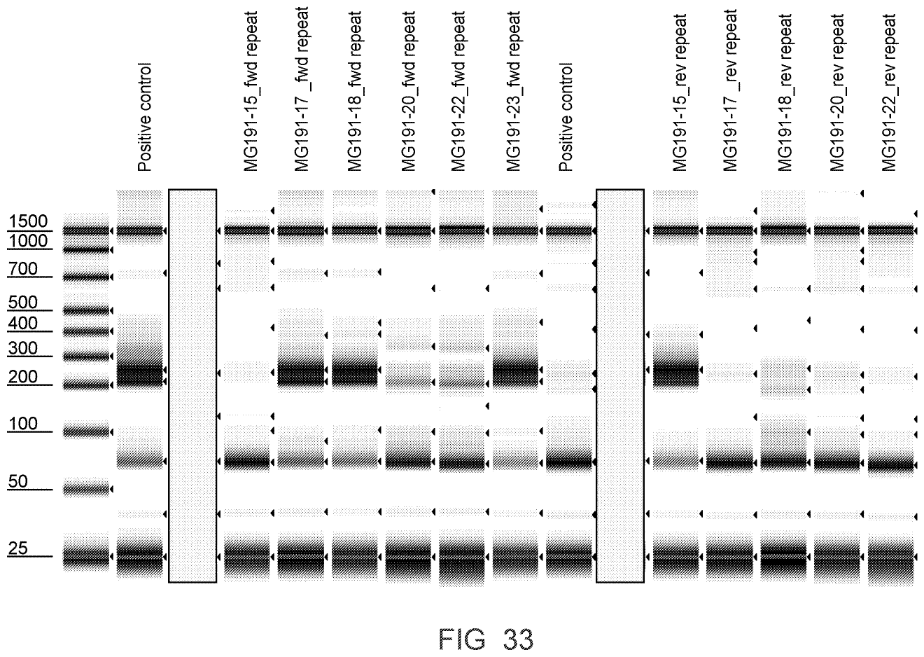

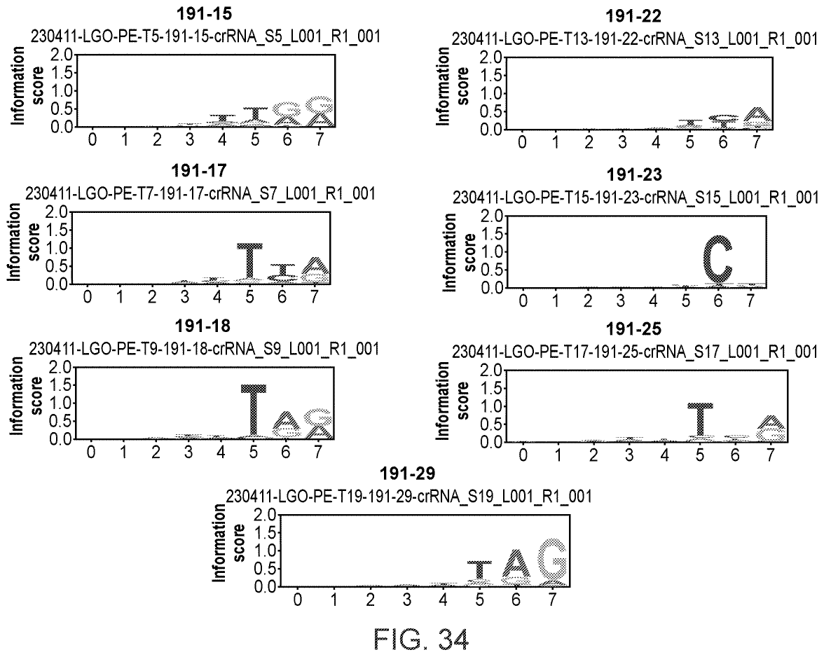

Active proteins that successfully cleaved the PAM library yielded a band around 205 bp by automated gel electrophoresis (FIG. 33). The identified PAMs (SEQ ID NOs: 1739-1745) are shown as sequence logos (FIG. 34). The preferred cut position on the target strand of the protospacer sequence complementary to the U40 spacer is listed in Table 15.

TABLE 15
MG191 nucleases preferred cutsites in the protospacer sequence.

| Nuclease | Minimal Array/Repeat | Cutsite |
|---|---|---|
| 191-15 | MG191-15_5U40_30_R | 21-22 |
| 191-17 | MG191-17_5U40_30_F | 22 |
| 191-18 | MG191-18_5U40_30_F | 22 |
| 191-20 | MG191-20_5U40_29_F | Activity unknown |
| 191-22 | MG191-22_5U40_30_F | Weak activity, 23 |
| 191-23 | MG191-23_5U40_30_F | 23 |
| 191-25 | MG191-25_5U40_30_F | 22 |
| 191-29 | MG191-29_5U40_30_F | 23 |

### MG191 Expression and Purification

Isolating pure and functional proteins is essential for extensive in vitro analysis of biochemical properties and mechanistic studies. MG191 candidates were expressed and purified to obtain proteins of sufficient quantity and quality for such characterizations. All constructs were expressed in E. coli. Constructs were expressed with an N-terminal MBP-fusion protein in the pMGD expression vector.

### Protein Expression

Protein expression plasmids were transformed into competent cells and cultured overnight in 25 mL growth media (1.6% tryptone, 1% yeast extract, 0.5% NaCl) with 100 μg/L Carbenicillin at 37° C. The next day, 10 mL from each overnight culture was used to inoculate 1000 mL growth media containing 100 μg/L Carbenicillin, and cultures were grown, shaking at 37° C. At OD600≈0.6, cultures were cooled on ice before induction with 0.5 mM IPTG and further incubation at 16° C. overnight, shaking, for approximately 18 hrs. Cultures were then harvested by centrifugation at 6,000×g for 10 min, and pellets were resuspended in Nickel_A Buffer (50 mM HEPES, 500 mM NaCl, 10 mM MgCl₂, 0.5 mM EDTA, 20 mM imidazole, 5% glycerol, pH 7.5)+EDTA-free protease inhibitors and stored at −80° C. Culture samples were taken pre- and post-induction, and cells were pelleted via centrifugation (15,000×g, 1.5 min) and resuspended in 100 μL 2× Laemmli Buffer per 1 OD cells.

### Protein Purification

All MG191 candidates were purified in the same manner. MG191-15 is shown here as an example. Proteins expressed in the pMGD vector have the following sequence architecture: 6×His-(GS)1-MBP-GSGSGGSGS-PSP-nucleoplasmin bipartite NLS-GGSGSGGS-MG119-X-GGSGGSG-SV40 NLS (Table 16).

TABLE 16
Sequence element glossary

| Element name | Element amino acid sequence |
|---|---|
| 6xHis | HHHHHH (SEQ ID NO: 1879) |
| (GS)ₙ | GS(SEQ ID NO: 1880) |
| PSP | LEVQFQGP (SEQ ID NO: 1883) |
| Nucleoplasmin | KRPAATKKAGQAKKKK (SEQ |
| bipartite NLS | ID NO: 1885) |
| SV40 NLS | PKKKRKV (SEQ ID NO: 1886) |

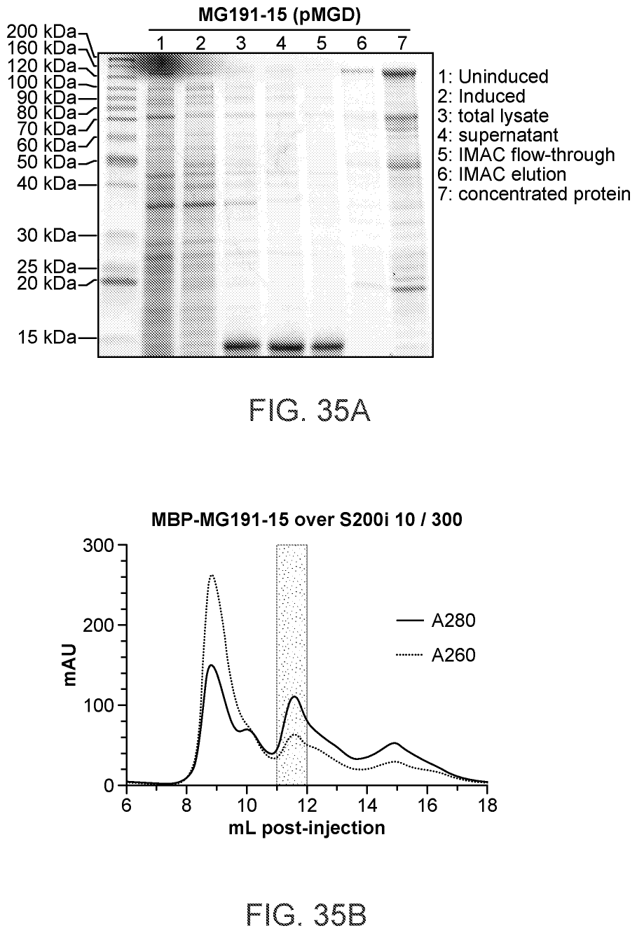

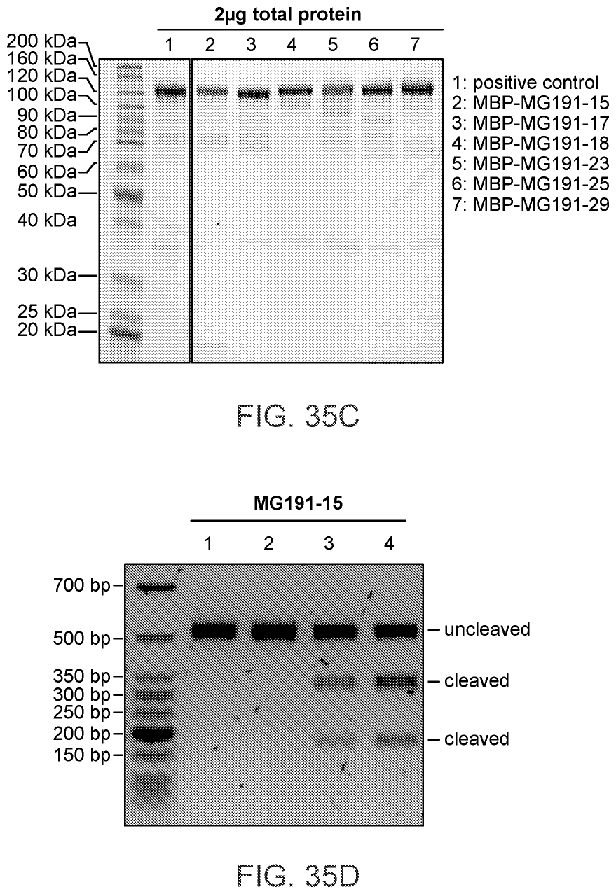

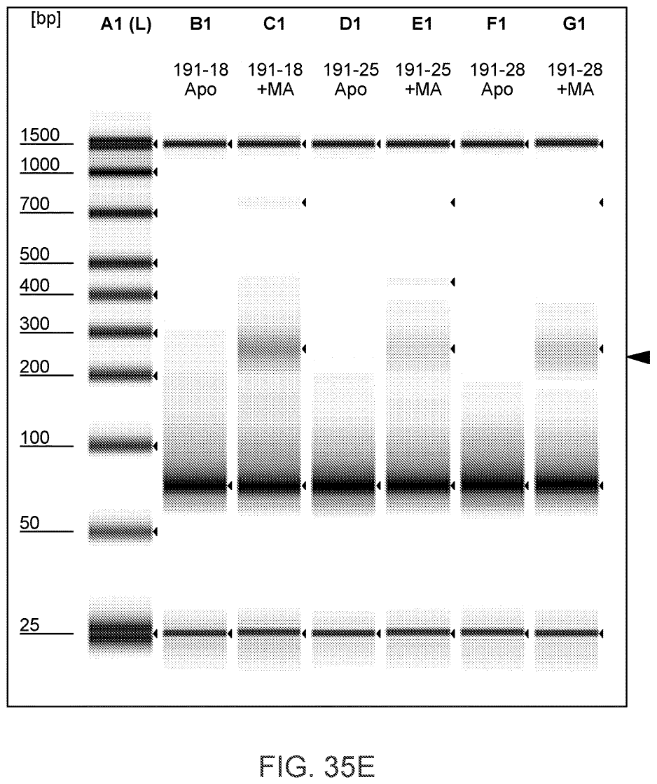

Cell pellets were thawed and the volume supplemented to 80 mL with Nickel_A buffer with 0.5% ß-octylglucoside. Samples were sonicated in an ice-water bath at 75% amplitude for a total processing time of 2 min using a 5 s on/15 s off cycle. Lysates were clarified by centrifugation at 30,000×g for 15 min, and supernatants batch bound to 2.5 mL Ni-NTA resin for ≥15 min. Samples were loaded onto a gravity column and washed with 30 CV Nickel wash Buffer, then eluted in 4 CV Nickel elution Buffer (Nickel wash buffer+250 mM imidazole) before concentrating in a 50 kDa MWCO concentrator. Samples were taken throughout the purification process and run on an SDS-PAGE protein gel, activated by 5 min of UV and imaged on a UV transilluminator. These gels were used to track the progress of purification throughout the protocol (FIG. 35A). Lastly, protein samples were then filtered through a 0.22 μm cellulose acetate membrane before loading onto an S200i 10/300 GL column and run into SEC buffer (20 mM HEPES, 500 mM NaCl, 10 mM MgCl₂, 0.5 mM EDTA, 5% glycerol, 0.5 mM TCEP, pH to 7.5) to further purify proteins (FIG. 35B). Peak fractions were pooled and concentrated in a 50 kDa MWCO concentrator. Each sample's absorbance at 280 nm was measured and used to calculate protein concentration; furthermore, the sample volume and concentration was used to estimate prep yield (nmol/L; Table 17). To estimate purity, 2 μg purified protein was run on an SDS-PAGE protein gel, activated by 5 min of UV and imaged on a UV transilluminator. (FIG. 35C). Densitometry was used to estimate the purity of each sample (Table 17). Through this protocol, purified proteins were obtained for each of the MG191 candidates.

TABLE 17
MG191 candidate purification yield and purity

| | Yield | Purity |
|---|---|---|
| Candidate | (nmol/L) | (%) |
| MG191-15 | 4.8 | 61 |
| MG191-17 | 9.7 | 74 |
| MG191-18 | 9.7 | 74 |
| MG191-23 | 21.8 | 67 |
| MG191-25 | 45.2 | 76 |
| MG191-29 | 6.6 | 83 |

### Nuclease Activity Evaluation Using Purified Proteins

Activity assays were performed to determine whether the MG191 purified proteins are capable of cleavage. This assay was performed to test activity of MG191-15, MG191-17, and MG191-23. The results of testing MG191-15 is shown as an example. These assays are performed by titrating RNP complex relative to a constant concentration of linear DNA substrate and measuring the DNA cleavage. Effector proteins were preincubated with a 1.5-fold molar excess of pre-crRNA (repeat-spacer-repeat-primer binding site) for 20 min at room temperature to form the ribonucleoprotein complex (RNP). Reactions were set up using 20 nM DNA substrate and a 5× (100 nM) and 25× (500 mM) molar excess of RNP over substrate. The reaction buffer composition was 10 mM Tris pH 7.5, 10 mM MgCl₂, 100 mM NaCl. The DNA substrate is 521 bp long and contains the PAM determined via NGS and the spacer specified in the pre-crRNA array. Successful cleavage results in fragments of approximately 171 and 350 bp. The reaction was incubated at 37° C. for 60 min, then incubated at 75° C. for 10 min. RNase A was added to each reaction (Cf=0.33 μg/μL), and samples were incubated at 37° C. for 10 min. Proteinase K was added to each reaction (Cf=60 units/mL), and samples were incubated at 55° C. for 15 min. The entirety of each reaction was then run on a 1.5% agarose gel a nucleic acid stain and imaged on a transilluminator. Active proteins produced product bands that migrated faster than the uncleaved reactant on an agarose gel (FIG. 35D). Percent cleaved substrate was calculated for each lane through densitometry analysis. These data show that all candidates, with the exception of MG191-17, are active as purified protein.

MG191 Testing Activity of Purified Protein with In Vitro Library Cleavage and Ligation Assay

The same in vitro assay described above was used to test for activity of MG191-18, MG191-25, and MG191-29 purified preps. In this assay, plasmid library DNA cleavage reactions were carried out by mixing 5 nM of the target library representing all possible 8N PAMs, 50 nM of effector protein, 50 nM of minimal array RNA, 10 mM Tris-HCl pH 7.9, 10 mM MgCl₂, 100 μg/ml BSA, and 50 mM NaCl at 37° C. for 30 minutes. Reactions were stopped and cleaned using PCR cleaning beads and eluted in water. 3 nM of the cleavage product ends were blunted with 3.33 μM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, and 20 U/μL T4 DNA ligase at room temperature for 20 minutes. The ligated products were amplified by PCR. Active proteins that successfully cleaved the PAM library yielded a band around 205 bp visualized by automated gel electrophoresis (FIG. 35E).

### Example 26—MG119-28 sgRNA is Truncated Successfully without Reducing Activity in Cells

Of the truncated guides that showed sustained high levels of cleavage in vitro, three were further tested in K562 cells targeting 9 different sites and showed a range of activity (Table 18) following the protocols described in Example 22. Guide scaffolds MG119-28_sg1_8 (SEQ ID NO: 703), MG119-28_sg1_5 (SEQ ID NO: 691), and MG119-28_sg1_8.5 (SEQ ID NO: 704) were used to design chemically modified guides targeting the spacers listed in Table 18, generating 27 sgRNAX.

TABLE 18
Target sites in hAAVS1

| | | WT targeting |
|---|---|---|
| | | guide |
| Site | Spacer | SEQ ID NO |
| AAVS1-B1 | GGGACTAGAAAGGTGAAGAG | 1524 |
| AAVS1-C3 | GGCAGCTCCCCTACCCCCCT | 1541 |
| AAVS1-C4 | GGTCTAACCCCCACCTCCTG | 1549 |
| AAVS1-D4 | GGCAGATTCCTTATCTGGTG | 1550 |
| AAVS1-F1 | GGAAGTGTAAGGAAGCTGCA | 1528 |
| AAVS1-F4 | CCAGAACCTCTAAGGTTTGC | 1552 |
| AAVS1-H3 | TCTGTCCCCTCCACCCCACA | 1546 |
| AAVS1-H5 | CCTGTGAGATAAGGCCAGTA | 1562 |

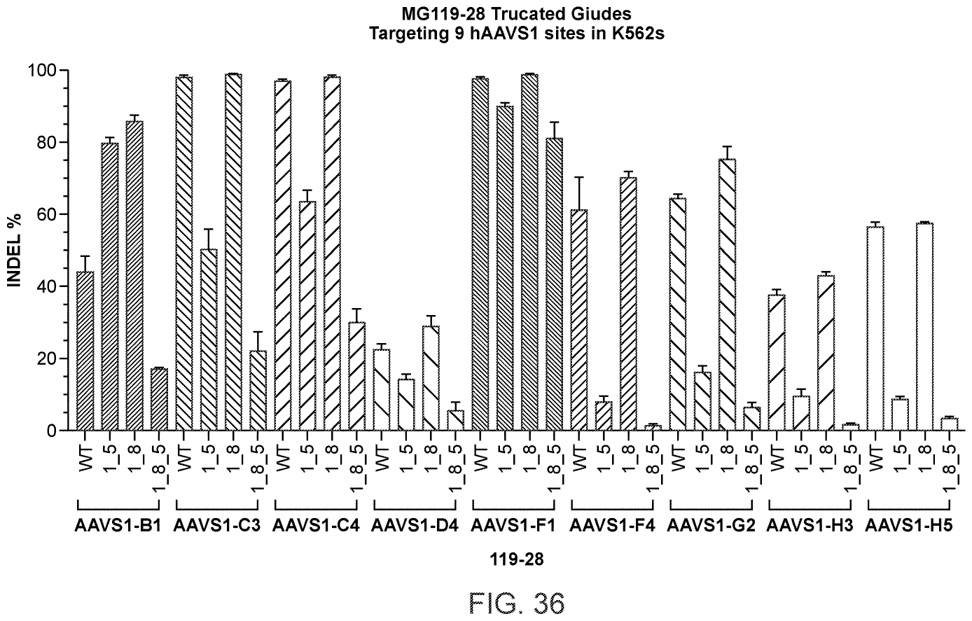

The NGS editing data showed that all designs were active in cells; however, guide scaffold 1_8 had similar or higher activity than the WT guide. Guide designs 1_5 and 1_8.5 had on average lower activity than the WT guide (FIG. 36).

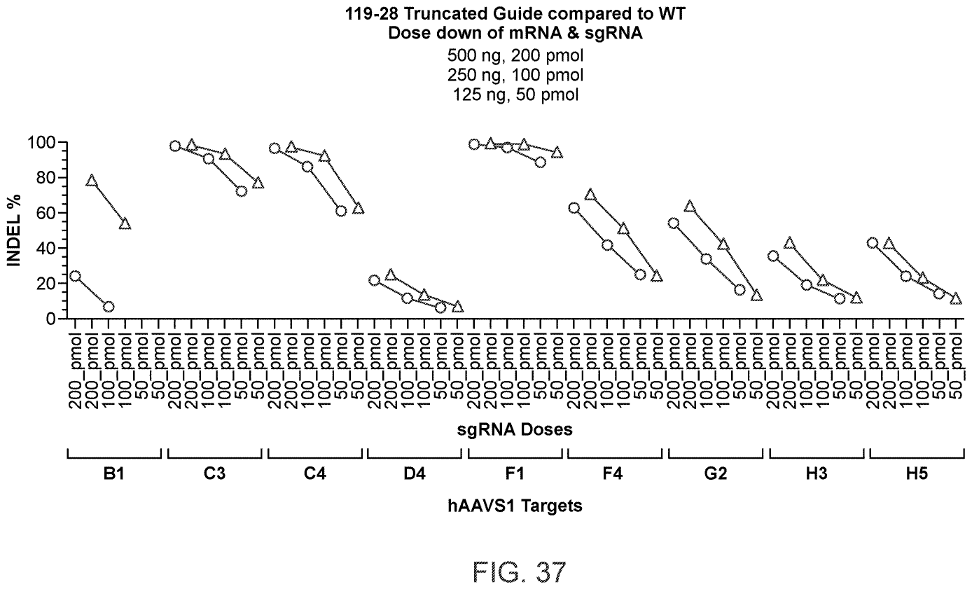

Further analysis of guide 1_8 was conducted by performing a dose titration of mRNA and guide 1_8 or WT into K562 cells (FIG. 37). These results demonstrate that sg1_8 generated equivalent or better levels of INDELs, even at sub-saturating doses of the guide, showing that this truncation to the sgRNA is not detrimental to activity. This truncation to WT sgRNA reduced the guide length by 15 nt.

### Example 27—Incremental Truncations of MG119-28 sgRNA and Nuclease Activity Testing in Cells

Activity was assessed of guides with incremental truncations throughout the repeat-anti-repeat region of the sgRNA. Guide designs included 20 nt spacers targeting five AAVS1 target sequences. The base sequence for all additional incremental truncations is SEQ ID NO: 1876, which is a sgRNA sequence modified from SEQ ID 703. K562 cells were grown and passaged in Iscove's Modified Dulbecco's Medium supplemented with 10% (v/v) fetal bovine serum (Gibco) at 37° C. with 5% CO2. 120K cells were transfected with 200 pmol of each guide and 500 ng of nuclease mRNA in a 96 well plate format as recommended by the Amaxa™ 4D-Nucleofector™ Protocol in a 4D-Nucleofector™ System (Lonza). Transfected cells were grown for 3 days, harvested, and gDNA was extracted with QuickExtract (Lucigen) per the manufacturer's instructions. PCR primers appropriate for use in NGS-based DNA sequencing were generated, optimized, and used to amplify the individual target sequences for each guide RNA and the extracted DNA as the template. PCR products were purified. The amplicons were sequenced on and analyzed.

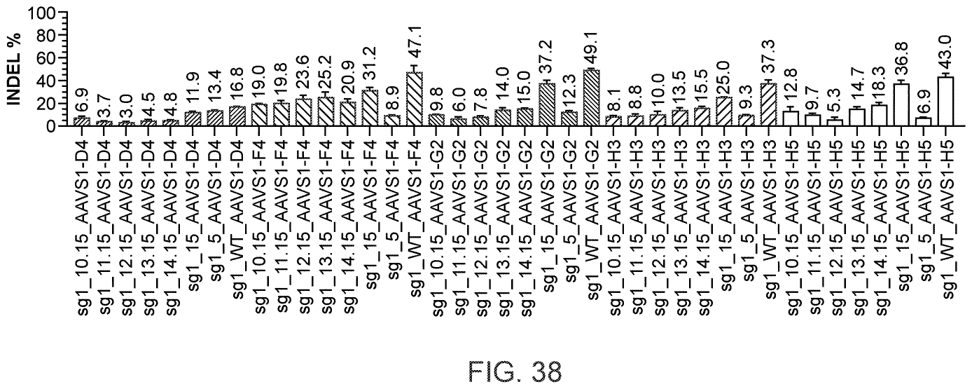

Analysis of the data shows that truncations to the targeted stem-loops decrease activity compared to the wild-type guide. Each guide is plotted against their corresponding INDELs and are compared against wild-type sgRNA activity (FIG. 38). At all five AAVS1 sites we observed detectable activity with each guide. Although activity is decreased relative to the WT guide, these alternate guides could be used if size or sequence of the guide was constrained.

### Example 28—Ancestral Sequence Reconstruction of MG191 Nucleases and In Vitro Activity Assay of Modern and Ancestral Sequences

### Ancestral Sequence Reconstruction of MG191 Nucleases

In order to generate further diversity of the MG191 family of mid-size type V nucleases, ancestral sequence reconstruction (ASR) algorithms were used. ASR is a computational technique that uses existing protein sequences and the relationships inferred between them to reconstruct potential ancestral sequences from ancient organisms. In brief, sequences were aligned using MAFFT with the Needleman-Wunsch algorithm for global alignment (Katoh and Standley 2013). Two phylogenetic trees were built using FastTree (Price, Dehal and Arkin 2010) using different sets of sequences, and both trees were rooted using known sequences as outgroups. Sequence reconstruction was done using the codeml package in PAML 4.8 (Yang 2007). Insertions and deletions were identified manually for each reconstructed node. Thirty-one ancestral sequences were reconstructed with high confidence and have the amino acid sequences shown in SEQ ID Nos: 1842-1872.

### In Vitro Cleavage Assay for Modern and Ancestral Candidates

### In Vitro Cleavage for PAM and Cutsite Determination

The MG191 family of nucleases are capable of processing their own crRNAs. To examine nuclease activity, modern MG191 nucleases MG191-1, -2, -5 and -28 (SEQ ID Nos: 1065, 1066, 1069, and 1115) were tested with their corresponding repeat sequences (SEQ ID No: 1873 or SEQ ID Nos: 1095, 1098 and 1119) and the U40 spacer listed in Table 19. Ancestral nucleases MG191-37, -38, -40, -41, -42, -48, -51, -53, and -62 (SEQ ID Nos: 1847, 1848, 1850-1852, 1858, 1861, 1863, and 1872) were tested with the minimal array for the closest modern homolog (SEQ IDs: 1732-1734, 1738, and 1874).

Table 20 shows active nuclease and minimal array combinations listed in this section.

5 nM of nuclease amplified DNA templates and 25 nM of promoter-annealed minimal array DNA templates mentioned above were expressed at 37° C. for 2 hours with PURExpress® In Vitro Protein Synthesis Kit (New England Biolabs Inc.). Plasmid library DNA cleavage reactions were carried out by mixing 5 nM of the target library representing all possible 8N PAMs with a 5-fold dilution of PURExpress expressions in 10 mM Tris-HCl pH 7.9, 10 mM MgCl2, 100 μg/ml BSA, and 50 mM NaCl (NEB 2.1 Buffer, NEB Inc.) at 37° C. for 2 hours. Reactions were stopped and cleaned and eluted in Tris EDTA pH 8.0 buffer. 3 nM of the cleavage product ends were blunted with 3.33 μM dNTPs, 1×T4 DNA ligase buffer, and 0.167 U/μL of Klenow Fragment at 25° C. for 15 minutes. 1.5 nM of the cleavage products were ligated with 150 nM adapters, 1×T4 DNA ligase buffer, 20 U/μL T4 DNA ligase at room temperature for 20 minutes. The ligated products were amplified by PCR with NGS primers and sequenced by NGS to obtain the PAM.

TABLE 19
Spacer sequence for tested guides

| Code | Sequence |
|---|---|
| U40 spacer | TGGAGATATCTTGAACCTTGCATCCCCGGA |
| (long) | (SEQ ID NO: 1901) |

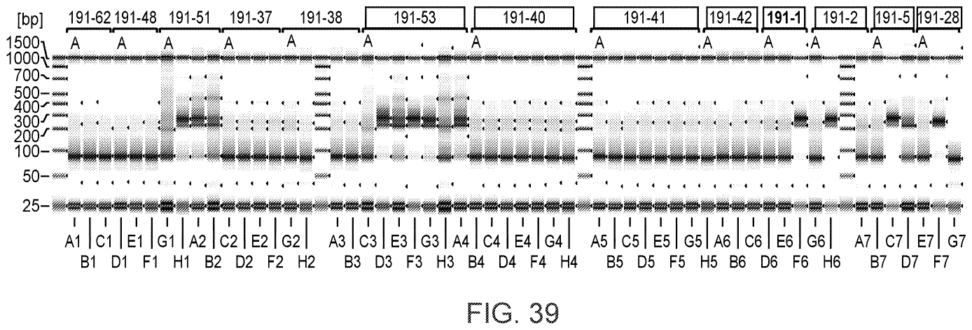

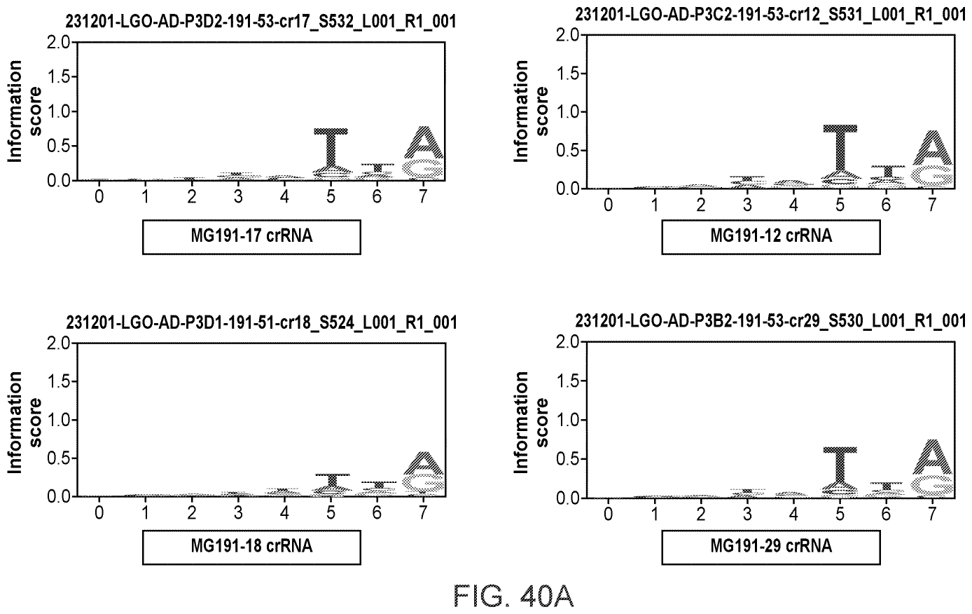

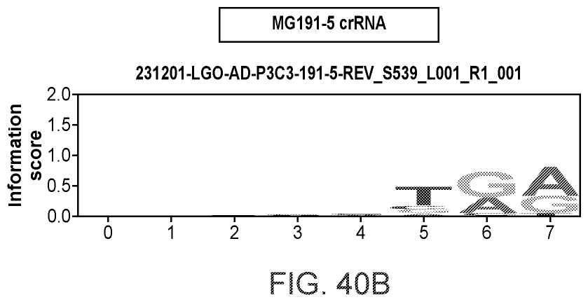

Active proteins that successfully cleaved the PAM library yielded a band around 200-300 bp in an TapeStation D1000 gel (Agilent Technologies Inc.) (FIG. 39). The PAM recognized by active modern and ancestral MG191 nucleases is provided in SEQ ID Nos: 1837-1841). Examples of the sequence logos made with Logo maker are shown in FIG. 40.

Modern nucleases MG191-1, MG191-2 and MG191-5 are active with tested minimal arrays and repeat orientations listed in Table 20 (SEQ ID Nos: 1867, 1095, and 1098). MG191-51 and MG191-53 ancestral nucleases were activated with multiple minimal arrays (current sequence table SEQ ID Nos: 1874, 1732 1734, and 1738). The preferred cut position on the target strand of the protospacer sequence complementary to the U40 spacer is listed in Table 20.

TABLE 20
Active MG191 nucleases preferred
cutsites in the protospacer sequence

| Nuclease | | Minimal Array/Repeat | Cutsite (TS) |
|---|---|---|---|
| NAncestral | 191-51 | MG191-15_5U40_30_R | 21-23 |
| Nucleases | | MG191-18_5U40_30_F | 23 |
| | | MG191-29_5U40_30_F | 22-23 |
| | 191-53 | MG191-12_5U40_30_R | 23 |
| | | MG191-18_5U40_30_F | 23 |
| | | MG191-17_5U40_30_F | 23 |
| | | MG191-29_5U40_30_F | 23 |
| Modern | 191-1 | MG191-1_5U40_31_R | 21-23 |
| Nucleases | 191-2 | MG191-2_5U40_30_F | 23 |
| | 191-5 | MG191-5_5U40_30_R | 23 |

MG191 crRNA Intersystem Swapability

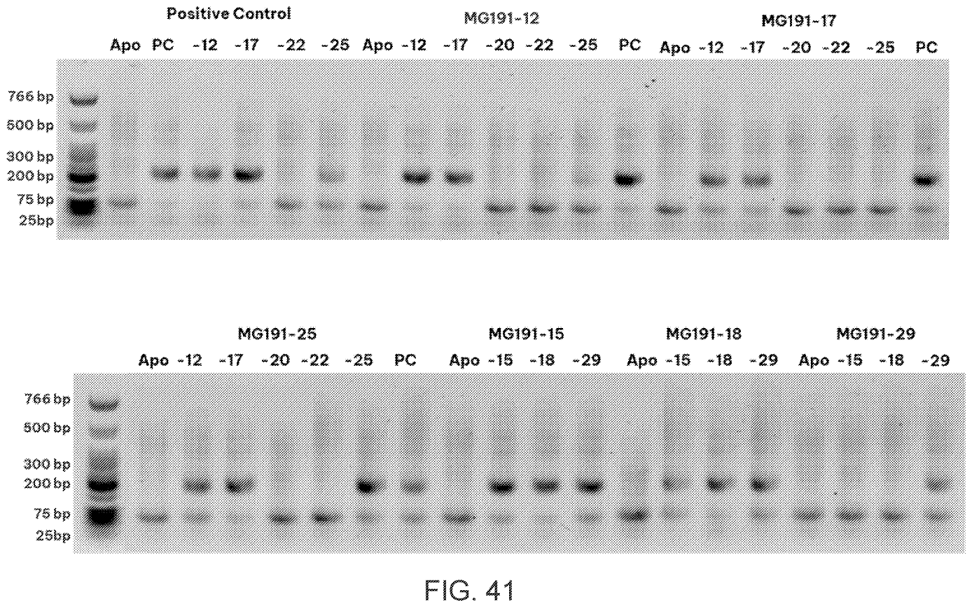

To optimize the execution of downstream testing in human cells, we tested repeat interchangeability within the MG191 nuclease family using the same in vitro cleavage assay described above. MG191-12, -15, -17, -18, -25 and -29 nucleases (SEQ ID Nos: 1076, 1079, 1081, 1082, 1089, 1116) with similar PAM sequences (SEQ ID Nos: 1739, 1741, 1744 and 1745) were tested with a combination of their corresponding repeats in minimal array format. Except for MG191-29, MG191 nucleases listed in Table 21 can use other crRNAs for in vitro cleavage (FIG. 41).

Purification of SUMO-fused MG191-12 and MG191-25 and evaluation of nuclease activity.

TABLE 21
MG191 activity with different minimal arrays

| | | PAM | In vitro |
|---|---|---|---|
| Nuclease | Minimal array | SEQ ID | activity |
| MG191-12 | MG191-12_5U40_30_R, | TTR | Active with |
| | MG191-17_5U40_30_F | | both repeats |
| MG191-17 | MG191-12_5U40_30_R, | TYR | Active with |
| | MG191-17_5U40_30_F | | both repeats |
| MG191-25 | MG191-12_5U40_30_R, | TnR | Active with |
| | MG191-17_5U40_30_F, | | three repeats |
| | MG191-25_5U40_30_F | | |
| MG191-15 | MG191-15_5U40_30_R, | tTRR | Active with |
| | MG191-18_5U40_30_F, | | three repeats |
| | MG191-29_5U40_30_F | | |
| MG191-18 | MG191-15_5U40_30_R, | TRR | Active with |
| | MG191-18_5U40_30_F, | | three repeats |
| | MG191-29_5U40_30_F | | |
| MG191-29 | MG191-29_5U40_30_F | TAG | Not active with |
| | | | other repeats |

MG191 candidates previously purified with MBP will have the MBP tag removed prior to any in-cell testing due to the large size of the MBP tag. To mitigate potential protein precipitation and degradation post MBP cleavage, MG191 candidates were expressed and purified with SUMO solubility tag, which does not need to be cleaved off due to its minimal size. Protein expressed with the SUMO tag has the following architecture: 6×His-(GS)1-SUMO-(GGSGS)₂-PSP-nucleoplasmin bipartite NLS-GGS-MG119-X-GP-SV40 NLS (Table 22). Densitometry was used to estimate the purity of the final sample of MG191-25 expressed with the SUMO construct compared to the same protein expressed with the MBP construct. The average purity of all collected fractions of MG191-25 expressed with SUMO tag at the final purification step was 57% compared to the 46% observed with MG191-25 expressed with MBP post tag-cleavage.

TABLE 22
Sequence element glossary

| Element name | Element amino acid sequence |
|---|---|
| PSP (PreScission | LEVLFQGP (SEQ ID NO: 1902) |
| Protease site) | |
| SUMO | SDSEVNQEAKPEVKPEVKPETHINLKVSDGSSEIFFKIKKTTP |
| | LRRLMEAFAKRQGKEMDSLRFLYDGIRIQADQTPEDLDMED |
| | NDIIEAHREQI (SEQ ID NO: 1900) |

Protein expression plasmids for SUMO-fused MG191-12 (SEQ ID NO: 1076) and MG191-25 were transformed into competent cells and cultured overnight in 25 mL 2×YT media (1.6% tryptone, 1% yeast extract, 0.5% NaCl) with 100 μg/L Carbenicillin at 37° C. The next day, 10 mL from each overnight culture was used to inoculate 1000 mL 2×YT media containing 100 μg/L Carbenicillin, and cultures were grown, shaking at 37° C. At OD600≈0.6, cultures were cooled on ice before induction with 0.5 mM IPTG and further incubation at 16° C. overnight, shaking, for approximately 18 hrs. Cultures were then harvested by centrifugation at 6,000×g for 10 min, and pellets were resuspended in Nickel_A Buffer (50 mM HEPES, 500 mM NaCl, 10 mM MgCl2, 0.5 mM EDTA, 20 mM imidazole, 5% glycerol, pH 7.5)+protease inhibitors and stored a −80° C. Culture samples were taken pre- and post-induction, and cells were pelleted via centrifugation (15,000×g, 1.5 min) and resuspended in 100 μL 2× Laemmli Buffer per 1 OD cells.

### Protein Purification

SUMO-fused MG191-12 and MG191-25 candidates were purified in the same manner by which MBP-tagged proteins were purified. MG191-12 is shown as an example. Cell pellets were thawed and the volume supplemented to 80 mL with Nickel_A buffer with 0.5% β-octylglucoside (P1P1P1, CI-00234). Samples were sonicated in an ice-water bath at 75% amplitude for a total processing time of 2.5 min using a 5 s on/15 s off cycle. Lysates were clarified by centrifugation at 30,000×g for 15 min, and filtered through a 0.2 μM aPES membrane before being loaded onto a 5 mL HisTrap Fast Flow column for immobilized metal-affinity chromatography (IMAC). The column was washed with 10 column volume (CV) of Nickel A buffer before going through an elution phase of 8 CV with increasing concentration of Nickel B Buffer (Nickel_A Buffer+500 mM imidazole) to reach 100% Nickel B Buffer at the end of the elution phase.

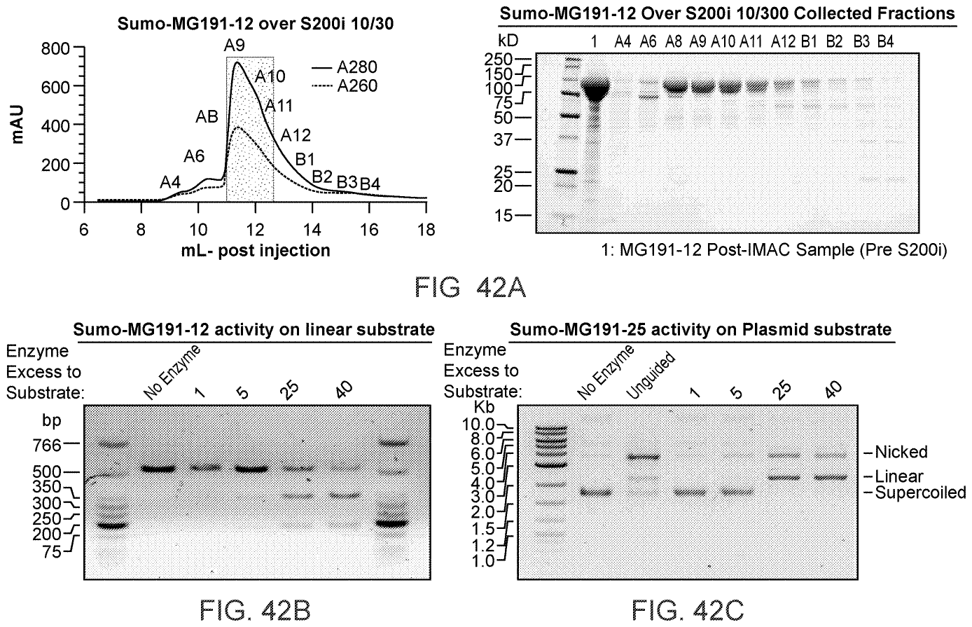

Eluted fractions with relatively low impurities were pooled and concentrated in a 50 kDa MWCO concentrator. The concentrated protein samples were then filtered through a 0.22 μm cellulose acetate membrane before loading onto an S200i 10/300 GL column (and run into SEC buffer (50 mM HEPES, 200 mM NaCl, 10 mM MgCl₂, 0.5 mM EDTA, 5% glycerol, 0.5 mM TCEP, pH to 7.5) to further purify proteins. Samples were taken from fraction throughout the size-exclusion purification process and run on an SDS-PAGE protein gel, which was imaged in the stain-free channel following 5 min UV activation. These gels were used to track elution of the protein of interest through the protocol (FIG. 42A). Peak fractions with relatively low impurities were pooled and concentrated in a 50 kDa MWCO concentrator. Each sample's absorbance at 280 nm was measured and used to calculate protein concentration. The sample volume and concentration were used to estimate prep yield. The results of the purification process are captured in Table 23.

TABLE 23
MG191 candidate purification yield

| | Yield |
|---|---|
| Candidate | (nmol/L) |
| MG191-12 | 45.4 |
| MG191-25 | 12.00 |

### Testing In Vitro Cleavage Activity of Purified Protein

The cleavage activity of SUMO-fused sample protein aliquots was determined in either a linear DNA substrate (FIG. 42B) or plasmid substrate (FIG. 42C; results using MG191-25 are shown) cleavage assay. Effector proteins were preincubated with a 1.5-fold molar excess of sgRNA for 20 min at room temperature to form the ribonucleoprotein complex (RNP). Reactions were set up using 25 nM linear DNA substrate or 5 nM plasmid DNA substrate and RNP at 0 and 40× molar excess over substrate. The reaction buffer composition was 10 mM Tris pH 7.5, 10 mM MgCl2, 100 mM NaCl. For the linear DNA cleavage assay, the substrate is 522 bp long. Successful cleavage results in fragments of 172 and 350 bp. For the plasmid substrate cleavage assay, the “Unguided” condition had no sgRNA and 40× molar excess of enzyme over substrate added in the RNP-forming step. The plasmid substrate is 2,218 bp long but would migrate faster than its actual size due its supercoiled plasmid form. A successful cleavage results in a linearized fragment that migrated slower than uncleaved plasmid. The reaction was incubated at 37° C. for added to each reaction (Cf=60 units/mL), and samples were incubated at 55° C. for 15 min. The entirety of each reaction was then run on a 1.5% agarose gel with GelGreen dye (Biotium, #41005) (FIG. 42B and FIG. 42C) and imaged.

In vitro cleavage activity was determined by the appearance of fragments that migrated slower than uncleaved plasmid. MG191-12 was active on linear substrate (FIG. 42B) and MG191-25 was only active on plasmid substrate (FIG. 42C).

### REFERENCES FOR EXAMPLE 28

Katoh K, Standley D M. MAFFT Multiple Sequence Alignment Software Version 7: Improvements in Performance and Usability. Mol Biol Evol 2013; 30:772-80. Price M N, Dehal P S, Arkin A P. FastTree 2—Approximately Maximum-Likelihood Trees for Large Alignments. Plos One 2010; 5:e9490. Yang Z. PAML 4: Phylogenetic Analysis by Maximum Likelihood. Mol Biol Evol 2007; 24:1586-91).

TABLE 19
Protein and nucleic acid sequences referred to herein

| | SEQ | | | | | |
|---|---|---|---|---|---|---|
| | ID | | | | Other | |
| Cat. | NO | Description | Type | Organism | Information | Sequence |
| MG119-137 | | MG119-137 | Nucleotide | Artificial | Uncultivated | CCn |
| effectors PAM (5′) | | PAM (5′) | | sequence | organism with | |
| | | | | | predicted | |
| | | | | | taxonomy: | |
| | | | | | Clostridiales | |
| MG119-1 effector | 647 | MG119- | Nucleotide | Artificial | R1 truncation | AUAGGGCGACUUGGCGUCCUUAAACCGAGCUCGGUAAGGUAAAUCCGUAA |
| sgRNA | | 1_sg1_1 | | sequence | | GGAUAAUCCACCCCGGCGAAAUGCCGGCCACUAAAAUGCAUAGAAAUAUG |
| | | | | | | CACAAGUGGAAGU |
| MG119-1 effector | 648 | MG119- | Nucleotide | Artificial | R1 truncation | AUAGGGCGACUUGGCGUCCUUAAAUAAGGUAAAUCCGUAAGGAUAAUCCA |
| sgRNA | | 1_sg1_2 | | sequence | | CCCCGGCGAAAUGCCGGCCACUAAAAUGCAUAGAAAUAUGCACAAGUGGA |
| | | | | | | AGU |
| MG119-1 effector | 649 | MG119- | Nucleotide | Artificial | R1 truncation | AUAGGGCGACUUGGCGUCCUUAAACCGAGAAGUGCAUCAGAAGGUUUACA |
| sgRNA | | 1_sg1_3 | | sequence | | UAGAUGUAACACUCGGUAAGGUAAAUCCGUAAGGAUAAUCCACCCCGGCG |
| | | | | | | AAAUGCCGGCCACUAGUGGAAGU |
| MG119-1 effector | 650 | MG119- | Nucleotide | Artificial | R1 truncation | AUAGGGCGACUUGGCGUCCUUAAACCGAGAAGUGCAUCAGAAGGUUUACA |
| sgRNA | | 1_sg1_4 | | sequence | | UAGAUGUAACACUCGGUAAGGUAAAUCCGUAAGGAUAAUCCACCCCGGCG |
| | | | | | | AAAUGCCGGCGAAGU |
| MG119-1 effector | 651 | MG119- | Nucleotide | Artificial | R1 truncation | AUAGGGCGACUUGGCGUCCUUAAACCGAGAAGUGCAUCAGAAGGUUUACA |
| sgRNA | | 1_sg1_5 | | sequence | | UAGAUGUAACACUCGGUAAGGUAAAUCCGUAAGGAUAAUCCACCCGCCAC |
| | | | | | | UAAAAUGCAUAGAAAUAUGCACAAGUGGAAGU |
| MG119-1 effector | 652 | MG119- | Nucleotide | Artificial | R1 truncation | AUAGGGCGACUUGGCGUCCUUAAACCGAGAAGUGCAUCAGAAGGUUUACA |
| sgRNA | | 1_sg1_6 | | sequence | | UAGAUGUAACACUCGGUAAGGCCACCCCGGCGAAAUGCCGGCCACUAAAA |
| | | | | | | UGCAUAGAAAUAUGCACAAGUGGAAGU |
| MG119-1 effector | 653 | MG119- | Nucleotide | Artificial | R1 truncation | AUAGGGCGACUUGGCGUCCUUAAACCGAGCUCGGUAAGGUAAAUCCGUAA |
| sgRNA | | 1_sg1_1.3 | | sequence | | GGAUAAUCCACCCCGGCGAAAUGCCGGCCACUAGUGGAAGU |
| MG119-1 effector | 654 | MG119- | Nucleotide | Artificial | R1 truncation | AUAGGGCGACUUGGCGUCCUUAAACCGAGCUCGGUAAGGUAAAUCCGUAA |
| sgRNA | | 1_sg1_1.5 | | sequence | | GGAUAAUCCACCCGCCACUAAAAUGCAUAGAAAUAUGCACAAGUGGAAGU |
| MG119-2 effector | 655 | MG119- | Nucleotide | Artificial | R0 (untruncated) | UACACAUCAUUAGAUGUGAGGGCAAUUUAAUGUCCUAAUAUUGAGGGUA [symbol] |
| sgRNA | | 2_sg2 | | sequence | | UAGGUAACAACACUUUGUUCUAAUAGAACAGUGUCUAAUAACCACUCGA [symbol] |
| | | | | | | AAGACCAUAUAAAUGAAAAUUUGUAUGGUUAUAAAAC |
| MG119-2 effector | 656 | MG119- | Nucleotide | Artificial | R1 truncation | UACACAUCAUUAGAUGUGAGGGCAAUUUAAUGUCCUAAUAUUGAGGGUA [symbol] |
| sgRNA | | 2_sg2_1 | | sequence | | UAGCUAAUAACCACUCGAUAAGACCAUAUAAAUGAAAAUUUGUAUGGUU [symbol] |
| | | | | | | UAAAAC |
| MG119-2 effector | 657 | MG119- | Nucleotide | Artificial | R1 truncation | UACACAUCAUUAGAUGUGAGGGCAAUUUAAUGUCCUAAUGACCAUAUAAA |
| sgRNA | | 2_sg2_2 | | sequence | | UGAAAAUUUGUAUGGUUAUAAAAC |
| MG119-2 effector | 658 | MG119- | Nucleotide | Artificial | R1 truncation | UACACAUCAUUAGAUGUGAGGGCAAUUUAAUGUCCUAAUAUUGAGGGUAU |
| sgRNA | | 2_sg2_3 | | sequence | | UAGGUAACAACACUUUGUUCUAAUAGAACAGUGUCUAAUAACCACUCGAU |
| | | | | | | AAGUAUAAAAC |
| MG119-2 effector | 659 | MG119- | Nucleotide | Artificial | R1 truncation | UACGAGGGCAAUUUAAUGUCCUAAUAUUGAGGGUAUUAGGUAACAACACU |
| sgRNA | | 2_sg2_4 | | sequence | | UUGUUCUAAUAGAACAGUGUCUAAUAACCACUCGAUAAGACCAUAUAAAU |
| | | | | | | GAAAAUUUGUAUGGUUAUAAAAC |
| MG119-2 effector | 660 | MG119- | Nucleotide | Artificial | R1 truncation | UACACAUCAUUAGAUGUGAUAAUAUUGAGGGUAUUAGGUAACAACACUUU |
| sgRNA | | 2_sg2_5 | | sequence | | GUUCUAAUAGAACAGUGUCUAAUAACCACUCGAUAAGACCAUAUAAAUGA |
| | | | | | | AAAUUUGUAUGGUUAUAAAAC |
| MG119-2 effector | 661 | MG119- | Nucleotide | Artificial | R1 truncation | UACACAUCAUUAGAUGUGAUAAUAUUGAGGGUAUUAGCUAAUAACCACUC |
| sgRNA | | 2_sg2_1.5 | | sequence | | GAUAAGACCAUAUAAAUGAAAAUUUGUAUGGUUAUAAAAC |
| MG119-2 effector | 662 | MG119- | Nucleotide | Artificial | R1 truncation | UACACAUCAUUAGAUGUGAGGGCAAUUUAAUGUCCUAAUAUUGAGGGUAU |
| sgRNA | | 2_sg2_1.6 | | sequence | | UAGCUAAUAACCACUCGAUAAGACCAUAUGAAAGUAUGGUUAUAAAAC |
| MG119-2 effector | 663 | MG119- | Nucleotide | Artificial | R2 truncation | UACGAGGGCAAUUUUAUGUCCUAAUAUUGAGGGUAUUAGGUAACAACACU |
| sgRNA | | 2_sg2_4.6 | | sequence | | UUGUUUUAAUAGAACAGUGUCUAAUAACCACUCGAUAAGACCAUAUGAAA |
| | | | | | | GUAUGGUUAUAAAAC |
| MG119-2 effector | 664 | MG119- | Nucleotide | Artificial | R2 truncation | UACGAGGGCAAUUUUAUGUCCUAAUAUUGAGGGUAUUAGGUAACAACACU |
| sgRNA | | 2_sg2_4.6.7 | | sequence | | UUGUUUGAACAGUGUCUAAUAACCACUCGAUAAGACCAUAUGAAAGUAUG |
| | | | | | | GUUAUAAAAC |
| MG119-2 effector | 665 | MG119- | Nucleotide | Artificial | R2 truncation | UACGAGGGCAAUUUUAUGUCCUAAUAUUGAGGGUAUUAGGUAACAACACU |
| sgRNA | | 2_sg2_4.6.8 | | sequence | | UUGUACAGUGUCUAAUAACCACUCGAUAAGACCAUAUGAAAGUAUGGUUA |
| | | | | | | UAAAAC |
| MG119-2 effector | 666 | MG119- | Nucleotide | Artificial | R2 truncation | UACGAGGGCAAUUUUAUGUCCUAAUAUUGAGGGUAUUAGGUAACAACACU |
| sgRNA | | 2_sg2_4.6.9 | | sequence | | UUAGUGUCUAAUAACCACUCGAUAAGACCAUAUGAAAGUAUGGUUAUAA |
| | | | | | | AC |
| MG119-2 effector | 667 | MG119- | Nucleotide | Artificial | R2 truncation | UACGAGGGCAAUUUUAUGUCCUAAUAUUGAGGGUAUUAGGUAACAACAC [symbol] |
| sgRNA | | 2_sg2_4.6. | | sequence | | GUGUCUAAUAACCACUCGAUAAGACCAUAUGAAAGUAUGGUUAUAAAAC [symbol] |
| | | 10 | | | | |
| MG119-2 effector | 668 | MG119- | Nucleotide | Artificial | R2 truncation | UACGAGGGCAAUUUUAUGUCCUAAUAUUGAGGGUAUUAGGUAACAACAU [symbol] |
| sgRNA | | 2_sg2_4.6. | | sequence | | UCUAAUAACCACUCGAUAAGACCAUAUGAAAGUAUGGUUAUAAAAC |
| | | 11 | | | | |
| MG119-2 effector | 669 | MG119- | Nucleotide | Artificial | R2 truncation | UACGAGGGCAAUUUAAUGUCCUAAUAUUGAGGGUAUUAGGUAACAACAC [symbol] |
| sgRNA | | 2_sg2_4.6_i | | sequence | | UUGUUCUAAUAGAACAGUGUCUAAUAACCACUCGAUAAGACCAUAUGAA [symbol] |
| | | | | | | GUAUGGUUAUAAAAC |
| MG119-2 effector | 670 | MG119- | Nucleotide | Artificial | R2 truncation | UACGAGGGCAAUUUAAUGUCCUAAUAUUGAGGGUAUUAGGUAACAACACU |
| sgRNA | | 2_sg2_4.6.7 | | sequence | | UUGUUCGAACAGUGUCUAAUAACCACUCGAUAAGACCAUAUGAAAGUAUG |
| | | _i | | | | GUUAUAAAAC |
| MG119-2 effector | 671 | MG119- | Nucleotide | Artificial | R2 truncation | UACGAGGGCAAUUUAAUGUCCUAAUAUUGAGGGUAUUAGGUAACAACACU |
| sgRNA | | 2_sg2_4.6.8_ | | sequence | | UUGUACAGUGUCUAAUAACCACUCGAUAAGACCAUAUGAAAGUAUGGUUA |
| | | i | | | | UAAAAC |
| MG119-2 effector | 672 | MG119- | Nucleotide | Artificial | R2 truncation | UACGAGGGCAAUUUAAUGUCCUAAUAUUGAGGGUAUUAGGUAACAACACU |
| sgRNA | | 2_sg2_4.6.9 | | sequence | | UUAGUGUCUAAUAACCACUCGAUAAGACCAUAUGAAAGUAUGGUUAUAAA |
| | | _i | | | | AC |
| MG119-2 effector | 673 | MG119- | Nucleotide | Artificial | R2 truncation | UACGAGGGCAAUUUAAUGUCCUAAUAUUGAGGGUAUUAGGUAACAACACU |
| sgRNA | | 2_sg2_4.6.1 | | sequence | | GUGUCUAAUAACCACUCGAUAAGACCAUAUGAAAGUAUGGUUAUAAAAC |
| | | 0_i | | | | |
| MG119-2 effector | 674 | MG119- | Nucleotide | Artificial | R2 truncation | UACGAGGGCAAUUUAAUGUCCUAAUAUUGAGGGUAUUAGGUAACAACAUG |
| sgRNA | | 2_sg2_4.6.1 | | sequence | | UCUAAUAACCACUCGAUAAGACCAUAUGAAAGUAUGGUUAUAAAAC |
| | | 1_i | | | | |
| MG119-3 effector | 675 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_1 | | sequence | | UUGGAACGCCUUAGUUUGAGGUUUGAAUAUAAAAAGUGAAAUAACACCUU |
| | | | | | | CAAACACUCAAAAAAUUCAUUUAGAAAUAAAUGAAUAUCCAAC |
| MG119-3 effector | 676 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_2 | | sequence | | UUGGAACGCCUUAGUUUGAGGUUUGAAUAUAAAAAGCCUUCAAACACUCA |
| | | | | | | AAAAAUUCAUUUAGAAAUAAAUGAAUAUCCAAC |
| MG119-3 effector | 677 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_3 | | sequence | | UUGGAACGCCUUAGUUUGAGGUUUGAAGGAGUUCAAACACUCAAAAAAUU |
| | | | | | | CAUUUAGAAAUAAAUGAAUAUCCAAC |
| MG119-3 effector | 678 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_4 | | sequence | | UUGGAACGCCUUAGUUGAAACAAAUGGAGAUUUGUAACAAAAAUUCAUU [symbol] |
| | | | | | | AGAAAUAAAUGAAUAUCCAAC |
| MG119-3 effector | 679 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_5 | | sequence | | UUGGAACGCCUUAGUUGAAUAUAAAAAGUGAAACAAAUGGAGAUUUGUA |
| | | | | | | ACACCUUCAAAAAUUCAUUUAGAAAUAAAUGAAUAUCCAAC |
| MG119-3 effector | 680 | MG119- | Nucleotide | Artificial | R1 truncation | GUUAAAAUUCUAGAUAUGAUUAUUAUAUUCCCUUUAUGGGAAUAGUUUG |
| sgRNA | | 3_sg1_6 | | sequence | | AGGUUUGAAUAUAAAAAGUGAAACAAAUGGAGAUUUGUAACACCUUCAA |
| | | | | | | ACACUCAAAAAAUUCAUUUAGAAAUAAAUGAAUAUCCAAC |
| MG119-3 effector | 681 | MG119- | Nucleotide | Artificial | R1 truncation | GUUAAAAUUCUAGAUAUGAUUAUUAUAUUAAGGGUGUGUUGGAACGCCU |
| sgRNA | | 3_sg1_7 | | sequence | | UAGUUUGAGGUUUGAAUAUAAAAAGUGAAACAAAUGGAGAUUUGUAACA |
| | | | | | | CCUUCAAACACUCAAAAAAUUCAUUUAGAAAUAAAUGAAUAUCCAAC |
| MG119-3 effector | 682 | MG119- | Nucleotide | Artificial | R1 truncation | GUUAAAAUUCUAGAUUCCCUUUAUGGGAAGGGUGUGUUGGAACGCCUUAG |
| sgRNA | | 3_sg1_8 | | sequence | | UUUGAGGUUUGAAUAUAAAAAGUGAAACAAAUGGAGAUUUGUAACACCU |
| | | | | | | UCAAACACUCAAAAAAUUCAUUUAGAAAUAAAUGAAUAUCCAAC |
| MG119-3 effector | 683 | MG119- | Nucleotide | Artificial | R1 truncation | GUUAAAAUUCUAGAUAUGAUUAUUAUAUUCCCUUUAUGGGAAGGGUGUG |
| sgRNA | | 3_sg1_9 | | sequence | | UUGGAACGCCUUAGUUUGAGGUUUGAAUAUAAAAAGUGAAACAAAUGGA |
| | | | | | | GAUUUGUAACACCUUCAAACACUCAAAUAUCCAAC |
| MG119-3 effector | 684 | MG119- | Nucleotide | Artificial | R1 truncation | GAUAUGAUUAUUAUAUUCCCUUUAUGGGAAGGGUGUGUUGGAACGCCUUA |
| sgRNA | | 3_sg1_10 | | sequence | | GUUUGAGGUUUGAAUAUAAAAAGUGAAACAAAUGGAGAUUUGUAACACC |
| | | | | | | UUCAAACACUCAAAAAAUUCAUUUAGAAAUAAAUGAAUAUCCAAC |
| MG119-3 effector | 685 | MG119- | Nucleotide | Artificial | R1 truncation | GUUAAAAUUCUAGAGGGUGUGUUGGAACGCCUUAGUUUGAGGUUUGAAU |
| sgRNA | | 3_sg1_11 | | sequence | | AUAAAAAGUGAAACAAAUGGAGAUUUGUAACACCUUCAAACACUCAAAAA |
| | | | | | | AUUCAUUUAGAAAUAAAUGAAUAUCCAAC |
| MG119-28 effector | 686 | MG119- | Nucleotide | Artificial | R0 (untruncated) | CAAACCCCUUCGGGGGAGGGCGCGUUGGAGCGCCUUAGUUUGAGGUGCAG |
| sgRNA | | 28_sg1 | | sequence | | AAUCAAAAAAACUGCGACGAUGGAGGUCGUUUCAGUCUCUGUACACUCAA |
| | | | | | | AAAAUUCACUUGAGAAAUCAAGUGAAUAUCCAAC |
| MG119-28 effector | 687 | MG119- | Nucleotide | Artificial | R1 truncation | CAAACCCCUUCGGGGGAGGGCGCGUUGGAGCGCCUUAGUUUGAGGUGCAG |
| sgRNA | | 28_sg1_1 | | sequence | | AUCUGUACACUCAAAAAAUUCACUUGAGAAAUCAAGUGAAUAUCCAAC |
| MG119-28 effector | 688 | MG119- | Nucleotide | Artificial | R1 truncation | CAAACCCCUUCGGGGGAGGGCGCGUUGGAGCGCCUUAGUUUGAGGUGCAG |
| sgRNA | | 28_sg1_2 | | sequence | | AAUCAAAAAAACUGCGUUCAGUCUCUGUACACUCAAAAAAUUCACUUGAG |
| | | | | | | AAAUCAAGUGAAUAUCCAAC |
| MG119-28 effector | 689 | MG119- | Nucleotide | Artificial | R1 truncation | CAAACCCCUUCGGGGGAUAGUUUGAGGUGCAGAAUCAAAAAAACUGCGAC |
| sgRNA | | 28_sg1_3 | | sequence | | GAUGGAGGUCGUUUCAGUCUCUGUACACUCAAAAAAUUCACUUGAGAAAU |
| | | | | | | CAAGUGAAUAUCCAAC |
| MG119-28 effector | 690 | MG119- | Nucleotide | Artificial | R1 truncation | CAAACGAGGGCGCGUUGGAGCGCCUUAGUUUGAGGUGCAGAAUCAAAAAA |
| sgRNA | | 28_sg1_4 | | sequence | | ACUGCGACGAUGGAGGUCGUUUCAGUCUCUGUACACUCAAAAAAUUCACU |
| | | | | | | UGAGAAAUCAAGUGAAUAUCCAAC |
| MG119-28 effector | 691 | MG119- | Nucleotide | Artificial | R1 truncation | CAAACCCCUUCGGGGGAGGGCGCGUUGGAGCGCCUUAGUUUGAGGUGCAG |
| sgRNA | | 28_sg1_5 | | sequence | | AAUCAAAAAAACUGCGACGAUGGAGGUCGUUUCAGUCUCUGUACACUCAA |
| | | | | | | AAAAUAUCCAAC |
| MG119-28 effector | 692 | MG119- | Nucleotide | Artificial | R1 truncation | CAAACGAGGGCGCGUUGGAGCGCCUUAGUUUGAGGUGCAGAUCUGUACAC |
| sgRNA | | 28_sg1_1.4 | | sequence | | UCAAAAAAUUCACUUGAGAAAUCAAGUGAAUAUCCAAC |
| MG119-28 effector | 693 | MG119- | Nucleotide | Artificial | R1 truncation | CAAACCCCUUCGGGGGAUAGUUUGAGGUGCAGAAUCAAAAAAACUGCGUU |
| sgRNA | | 28_sg1_2.3 | | sequence | | CAGUCUCUGUACACUCAAAAAAUUCACUUGAGAAAUCAAGUGAAUAUCCA |
| | | | | | | AC |
| MG119-28 effector | 694 | MG119- | Nucleotide | Artificial | R1 truncation | CAAACGAGGGCGCGUUGGAGCGCCUUAGUUUGAGGUGCAGAAUCAAAAAA |
| sgRNA | | 28_sg1_2.4 | | sequence | | ACUGCGUUCAGUCUCUGUACACUCAAAAAAUUCACUUGAGAAAUCAAGUG |
| | | | | | | AAUAUCCAAC |
| MG119-28 effector | 695 | MG119- | Nucleotide | Artificial | R2 truncation | CAAACGAGGGCGCGUUGGAGCGCCUUAGUUUGAGGUGCAGAAUCAAAAAA |
| sgRNA | | 28_sg1_4.5 | | sequence | | ACUGCGACGAUGGAGGUCGUUUCAGUCUCUGUACACUCAAAAAAUAUCCA |
| | | | | | | AC |
| MG119-28 effector | 696 | MG119- | Nucleotide | Artificial | R2 truncation | CAAACGAGGGCGCGUUGGAGCGCCUUAGUUUGAGGUGCAGAAAAAACUGC |
| sgRNA | | 28_sg1_4.5. | | sequence | | GACGAUGGAGGUCGUUUCAGUCUCUGUACACUCAAAAAAUAUCCAAC |
| | | 7 | | | | |
| MG119-28 effector | 697 | MG119- | Nucleotide | Artificial | R2 truncation | CAAACGAGGGCGCGUUGGAGCGCCUUAGUUUGAGGUGCAGAAUCAAAAAA |
| sgRNA | | 28_sg1_4.6 | | sequence | | ACUGCGACGAUGGAGGUCGUUUCAGUCUCUGUACACUCAAAAAAUUCACU |
| | | | | | | UGAGAAAUCAAGUGAAUC |
| MG119-28 effector | 698 | MG119- | Nucleotide | Artificial | R2 truncation | CAAACGAGGGCGCGUUGGAGCGCCUUAGUUUGAGGUGCAGAAAAAACUGC |
| sgRNA | | 28_sg1_4.7 | | sequence | | GACGAUGGAGGUCGUUUCAGUCUCUGUACACUCAAAAAAUUCACUUGAGA |
| | | | | | | AAUCAAGUGAAUAUCCAAC |
| MG119-28 effector | 699 | MG119- | Nucleotide | Artificial | R2 truncation | CAAACGAGGGCGCGUUGGAGCGCCUUAGUUUGAGGUGCAGAAUCAAAAAA |
| sgRNA | | 28_sg1_4.9 | | sequence | | ACUGCGACGAUGGAGGUCGUUUCAGUCUCUGUACACUCAAAAAC |
| MG119-28 effector | 700 | MG119- | Nucleotide | Artificial | R2 truncation | CAAACCCCUUCGGGGGAGGGCGCGUUGGAGCGCCUUAGUUUGAGGUGCAG |
| sgRNA | | 28_sg1_5.7 | | sequence | | AAAAAACUGCGACGAUGGAGGUCGUUUCAGUCUCUGUACACUCAAAAAAU |
| | | | | | | AUCCAAC |
| MG119-28 effector | 701 | MG119- | Nucleotide | Artificial | R2 truncation | CAAACCCCUUCGGGGGAGGGCGCGUUGGAGCGCCUUAGUUUGAGGUGCAG |
| sgRNA | | 28_sg1_6 | | sequence | | AAUCAAAAAAACUGCGACGAUGGAGGUCGUUUCAGUCUCUGUACACUCAA |
| | | | | | | AAAAUUCACUUGAGAAAUCAAGUGAAUC |
| MG119-28 effector | 702 | MG119- | Nucleotide | Artificial | R2 truncation | CAAACCCCUUCGGGGGAGGGCGCGUUGGAGCGCCUUAGUUUGAGGUGCAG |
| sgRNA | | 28_sg1_7 | | sequence | | AAAAAACUGCGACGAUGGAGGUCGUUUCAGUCUCUGUACACUCAAAAAAU |
| | | | | | | UCACUUGAGAAAUCAAGUGAAUAUCCAAC |
| MG119-28 effector | 703 | MG119- | Nucleotide | Artificial | R2 truncation | CAGGGCGCGUUGGAGCGCCUUAGUUUGAGGUGCAGAAUCAAAAAAACUG [symbol] |
| sgRNA | | 28_sg1_8 | | sequence | | GACGAUGGAGGUCGUUUCAGUCUCUGUACACUCAAAAAAUUCACUUGAGA |
| | | | | | | AAUCAAGUGAAUAUCCAAC |
| MG119-28 effector | 704 | MG119- | Nucleotide | Artificial | R2 truncation | CAGGGCGCGUUGGAGCGCCUUAGUUUGAGGUGCAGAAUCAAAAAAACUGC |
| sgRNA | | 28_sg1_8.5 | | sequence | | GACGAUGGAGGUCGUUUCAGUCUCUGUACACUCAAAAAAUAUCCAAC |
| MG119-28 effector | 705 | MG119- | Nucleotide | Artificial | R2 truncation | CAGGGCGCGUUGGAGCGCCUUAGUUUGAGGUGCAGAAAAAACUGCGACGA |
| sgRNA | | 28_sg1_8.5. | | sequence | | UGGAGGUCGUUUCAGUCUCUGUACACUCAAAAAAUAUCCAAC |
| | | 7 | | | | |
| MG119-28 effector | 706 | MG119- | Nucleotide | Artificial | R2 truncation | CAGGGCGCGUUGGAGCGCCUUAGUUUGAGGUGCAGAAUCAAAAAAACUGC |
| sgRNA | | 28_sg1_8.6 | | sequence | | GACGAUGGAGGUCGUUUCAGUCUCUGUACACUCAAAAAAUUCACUUGAGA |
| | | | | | | AAUCAAGUGAAUC |
| MG119-28 effector | 707 | MG119- | Nucleotide | Artificial | R2 truncation | CAGGGCGCGUUGGAGCGCCUUAGUUUGAGGUGCAGAAAAAACUGCGACGA |
| sgRNA | | 28_sg1_8.7 | | sequence | | UGGAGGUCGUUUCAGUCUCUGUACACUCAAAAAAUUCACUUGAGAAAUCA |
| | | | | | | AGUGAAUAUCCAAC |
| MG119-28 effector | 708 | MG119- | Nucleotide | Artificial | R2 truncation | CAGGGCGCGUUGGAGCGCCUUAGUUUGAGGUGCAGAAAAAACUGCGACGA |
| sgRNA | | 28_sg1_8.7. | | sequence | | UGGAGGUCGUUUCAGUCUCUGUACACUCAAAAAC |
| | | 9 | | | | |
| MG119-28 effector | 709 | MG119- | Nucleotide | Artificial | R2 truncation | CAGGGCGCGUUGGAGCGCCUUAGUUUGAGGUGCAGAAUCAAAAAAACUGC |
| sgRNA | | 28_sg1_8.9 | | sequence | | GACGAUGGAGGUCGUUUCAGUCUCUGUACACUCAAAAAC |
| MG119-28 effector | 710 | MG119- | Nucleotide | Artificial | R2 truncation | CAAACCCCUUCGGGGGAGGGCGCGUUGGAGCGCCUUAGUUUGAGGUGCAG |
| sgRNA | | 28_sg1_9 | | sequence | | AAUCAAAAAAACUGCGACGAUGGAGGUCGUUUCAGUCUCUGUACACUCAA |
| | | | | | | AAAC |
| MG119-28 effector | 711 | MG119- | Nucleotide | Artificial | R2 truncation | CAAACCCCUUCGGGGGAGGGCGCGUUGGAGCGCCUUAGUUUGAGGUGCAG |
| sgRNA | | 28_sg1_9.7 | | sequence | | AAAAAACUGCGACGAUGGAGGUCGUUUCAGUCUCUGUACACUCAAAAAC |
| MG119-32 effector | 712 | MG119- | Nucleotide | Artificial | R1 truncation | AUAUAGUCCCAUUAGGGAUUGGGCGUGUUGGAACGCCUUAGUUUGAGGUU |
| sgRNA | | 32_sg1_1 | | sequence | | UGAAACCAAAAGCGCACCUUCAAGCACUCAAAAAAUCCACUUAUAUGAAA |
| | | | | | | AUAUAAGGGGAUAUCCAACG |
| MG119-32 effector | 713 | MG119- | Nucleotide | Artificial | R1 truncation | AUAUAGUCCCAUUAGGGAUUGGGCGUGUUGGAACGCCUUAGUUUGAGGUU |
| sgRNA | | 32_sg1_2 | | sequence | | UGAGGAGUCAAGCACUCAAAAAAUCCACUUAUAUGAAAAUAUAAGGGGAU |
| | | | | | | AUCCAACG |
| MG119-32 effector | 714 | MG119- | Nucleotide | Artificial | R1 truncation | AUAUAGUCCCAUUAGGGAUUGGGCGUGUUGGAACGCCUUAGUGUGUAGAU |
| sgRNA | | 32_sg1_3 | | sequence | | GGAGGUCUGUACAAAAUCCACUUAUAUGAAAAUAUAAGGGGAUAUCCAAC |
| | | | | | | G |
| MG119-32 effector | 715 | MG119- | Nucleotide | Artificial | R1 truncation | AUAUAGUCCCAUUAGGGAUUUAGUUUGAGGUUUGAAACCAAAAGCGGUG [symbol] |
| sgRNA | | 32_sg1_4 | | sequence | | AGAUGGAGGUCUGUACCACCUUCAAGCACUCAAAAAAUCCACUUAUAUGA |
| | | | | | | AAAUAUAAGGGGAUAUCCAACG |
| MG119-32 effector | 716 | MG119- | Nucleotide | Artificial | RI truncation | AUAUGGGCGUGUUGGAACGCCUUAGUUUGAGGUUUGAAACCAAAAGCGGI |
| sgRNA | | 32_sg1_5 | | sequence | | GUAGAUGGAGGUCUGUACCACCUUCAAGCACUCAAAAAAUCCACUUAUAU |
| | | | | | | GAAAAUAUAAGGGGAUAUCCAACG |
| MG119-32 effector | 717 | MG119- | Nucleotide | Artificial | R1 truncation | AUAUAGUCCCAUUAGGGAUUGGGCGUGUUGGAACGCCUUAGUUUGAGGUI |
| sgRNA | | 32_sg1_6 | | sequence | | UGAAACCAAAAGCGGUGUAGAUGGAGGUCUGUACCACCUUCAAGCACUCA |
| | | | | | | AAAAACUUAUAUGAAAAUAUAAGUAUCCAACG |
| MG119-32 effector | 718 | MG119- | Nucleotide | Artificial | R1 truncation | AUAUAGUCCCAUUAGGGAUUGGGCGUGUUGGAACGCCUUAGUUUGAGGUU |
| sgRNA | | 32_sg1_7 | | sequence | | UGAAACCAAAAGCGGUGUAGAUGGAGGUCUGUACCACCUUCAAGCACUCA |
| | | | | | | AAAAAUCCGAAAGGAUAUCCAACG |
| MG119-32 effector | 719 | MG119- | Nucleotide | Artificial | R1 truncation | AUAUAGUCCCAUUAGGGAUUGGGCGUGUUGGAACGCCUUAGUUUGAGGUU |
| sgRNA | | 32_sg1_8 | | sequence | | UGAAACCAAAAGCGGUGUAGAUGGAGGUCUGUACCACCUUCAAGCACUCA |
| | | | | | | AAAAAUAUCCAACG |
| MG119-32 effector | 720 | MG119- | Nucleotide | Artificial | R0 (untruncated) | UUGAAAUUGCAGAUACAAGUUAUAUAGUCCCAUUAGGGAUUGGGCGUGUU |
| sgRNA | | 32_sg2 | | sequence | | GGAACGCCUUAGUUUGAGGUUUGAAACCAAAAGCGGUGUAGAUGGAGGUC |
| | | | | | | UGUACCACCUUCAAGCACUCAAAAAAUCCACUUAUAUGAAAAUAUAAGGG |
| | | | | | | GAUAUCCAACG |
| MG119-32 effector | 721 | MG119- | Nucleotide | Artificial | R1 truncation | UUGAAAUUGCAGAUACAAGUUAUAUAGUCCCAUUAGGGAUUGGGCGUGUU |
| sgRNA | | 32_sg2_1 | | sequence | | GGAACGCCUUAGUUUGAGGUUUGAAACCAAAAGCGCACCUUCAAGCACUC |
| | | | | | | AAAAAAUCCACUUAUAUGAAAAUAUAAGGGGAUAUCCAACG |
| MG119-32 effector | 722 | MG119- | Nucleotide | Artificial | R1 truncation | UUGAAAUUGCAGAUACAAGUUAUAUAGUCCCAUUAGGGAUUGGGCGUGUU |
| sgRNA | | 32_sg2_2 | | sequence | | GGAACGCCUUAGUUUGAGGUUUGAGGAGUCAAGCACUCAAAAAAUCCACU |
| | | | | | | UAUAUGAAAAUAUAAGGGGAUAUCCAACG |
| MG119-32 effector | 723 | MG119- | Nucleotide | Artificial | R1 truncation | UUGAAAUUGCAGAUACAAGUUAUAUAGUCCCAUUAGGGAUUGGGCGUGUU |
| sgRNA | | 32_sg2_3 | | sequence | | GGAACGCCUUAGUGUGUAGAUGGAGGUCUGUACAAAAUCCACUUAUAUGA |
| | | | | | | AAAUAUAAGGGGAUAUCCAACG |
| MG119-32 effector | 724 | MG119- | Nucleotide | Artificial | R1 truncation | UUGAAAUUGCAGAUACAAGUUAUAUAGUCCCAUUAGGGAUUUAGUUUGA |
| sgRNA | | 32_sg2_4 | | sequence | | GGUUUGAAACCAAAAGCGGUGUAGAUGGAGGUCUGUACCACCUUCAAGCA |
| | | | | | | CUCAAAAAAUCCACUUAUAUGAAAAUAUAAGGGGAUAUCCAACG |
| MG119-32 effector | 725 | MG119- | Nucleotide | Artificial | R1 truncation | UUGAAAUUGCAGAUACAAGUUAUAUGGGCGUGUUGGAACGCCUUAGUUUG |
| sgRNA | | 32_sg2_5 | | sequence | | AGGUUUGAAACCAAAAGCGGUGUAGAUGGAGGUCUGUACCACCUUCAAGC |
| | | | | | | ACUCAAAAAAUCCACUUAUAUGAAAAUAUAAGGGGAUAUCCAACG |
| MG119-32 effector | 726 | MG119- | Nucleotide | Artificial | R1 truncation | UUGAAAUUGCAGAUACAAGUUAUAUAGUCCCAUUAGGGAUUGGGCGUGU [symbol] |
| sgRNA | | 32_sg2_6 | | sequence | | GGAACGCCUUAGUUUGAGGUUUGAAACCAAAAGCGGUGUAGAUGGAGGU [symbol] |
| | | | | | | UGUACCACCUUCAAGCACUCAAAAAACUUAUAUGAAAAUAUAAGUAUCCA |
| | | | | | | ACG |
| MG119-32 effector | 727 | MG119- | Nucleotide | Artificial | R1 truncation | UUGAAAUUGCAGAUACAAGUUAUAUAGUCCCAUUAGGGAUUGGGCGUGU [symbol] |
| sgRNA | | 32_sg2_7 | | sequence | | GGAACGCCUUAGUUUGAGGUUUGAAACCAAAAGCGGUGUAGAUGGAGGU [symbol] |
| | | | | | | UGUACCACCUUCAAGCACUCAAAAAAUCCGAAAGGAUAUCCAACG |
| MG119-32 effector | 728 | MG119- | Nucleotide | Artificial | R1 truncation | UUGAAAUUGCAGAUACAAGUUAUAUAGUCCCAUUAGGGAUUGGGCGUGU [symbol] |
| sgRNA | | 32_sg2_8 | | sequence | | GGAACGCCUUAGUUUGAGGUUUGAAACCAAAAGCGGUGUAGAUGGAGGU [symbol] |
| | | | | | | UGUACCACCUUCAAGCACUCAAAAAAUAUCCAACG |
| MG119-54 effector | 729 | MG119- | Nucleotide | Artificial | R1 truncation | AACAGAUAUACGACACGAAGUAUAUUUACAUGGGGCUGUUGGAGGCGCCU |
| sgRNA | | 54_sg1_1 | | sequence | | UAGUUUGAGGUUCAGACAUAAAAAAUAUGUCUGCGCACUCGAAAAAUUCA |
| | | | | | | UCUCGAAAGGGAUGAAUAUCCAAC |
| MG119-54 effector | 730 | MG119- | Nucleotide | Artificial | R1 truncation | AACAGAUAUACGACACGAAGUAUAUUUACAUGGGGCUGUUGGAGGCGCCU |
| sgRNA | | 54_sg1_2 | | sequence | | UAGUUUGAGGUUCAGACAUAAAAAAUCCAGUCGGGAUGUCUGCGCACUCG |
| | | | | | | AAAAAUUCAUCUCGAAAGGGAUGAAUAUCCAAC |
| MG119-54 effector | 731 | MG119- | Nucleotide | Artificial | R1 truncation | AACAGAUAUACGACACGAAGUAUAUUUACAUGGGGCUGUUGGAGGCGCCU |
| sgRNA | | 54_sg1_3 | | sequence | | UAGUUUGAGGUUCAUGACAGUCGGUUAUGCGCACUCGAAAAAUUCAUCUC |
| | | | | | | GAAAGGGAUGAAUAUCCAAC |
| MG119-54 effector | 732 | MG119- | Nucleotide | Artificial | R1 truncation | AACAGAUAUACGACACGAAGUAUAUUUACAUGGGGCUGUUGGAGGCGCCU |
| sgRNA | | 54_sg1_4 | | sequence | | UAGUUAGACAUAAAAAAUCGUUAUGACAGUCGGUUAUAACGAUGUCUAAA |
| | | | | | | AAUUCAUCUCGAAAGGGAUGAAUAUCCAAC |
| MG119-54 effector | 733 | MG119- | Nucleotide | Artificial | R1 truncation | AACAGAUAUACGACACGAAGUAUAUUUACAUGGGGCUGUUGGAGGCGCCU |
| sgRNA | | 54_sg1_5 | | sequence | | UAGUUCGUUAUGACAGUCGGUUAUAACGAAAAAUUCAUCUCGAAAGGGAU |
| | | | | | | GAAUAUCCAAC |
| MG119-54 effector | 734 | MG119- | Nucleotide | Artificial | R1 truncation | AACAGAUAUACGACACGAAGUAUAUUUAUUUGAGGUUCAGACAUAAAAAA |
| sgRNA | | 54_sg1_6 | | sequence | | UCGUUAUGACAGUCGGUUAUAACGAUGUCUGCGCACUCGAAAAAUUCAUC |
| | | | | | | UCGAAAGGGAUGAAUAUCCAAC |
| MG119-54 effector | 735 | MG119- | Nucleotide | Artificial | R1 truncation | AACAUGGGGCUGUUGGAGGCGCCUUAGUUUGAGGUUCAGACAUAAAAAAU |
| sgRNA | | 54_sg1_7 | | sequence | | CGUUAUGACAGUCGGUUAUAACGAUGUCUGCGCACUCGAAAAAUUCAUCU |
| | | | | | | CGAAAGGGAUGAAUAUCCAAC |
| MG119-54 effector | 736 | MG119- | Nucleotide | Artificial | R1 truncation | AACAGAUAUACGACACGAAGUAUAUUUACAUGGGGCUGUUGGAGGCGCCU |
| sgRNA | | 54_sg1_8 | | sequence | | UAGUUUGAGGUUCAGACAUAAAAAAUCGUUAUGACAGUCGGUUAUAACGA |
| | | | | | | UGUCUGCGCACUCGAAAAAUAUCCAAC |
| MG119-54 effector | 737 | MG119- | Nucleotide | Artificial | R1 truncation | AACAGAUAUACGACACGAAGUAUAUUUACAUGGGGCUGUUGGAGGCGCCU |
| sgRNA | | 54_sg1_9 | | sequence | | UAGUUUGAGGUUCAGACAUAAAAAAUCGUUAUGACAGUCGGUUAUAACG [symbol] |
| | | | | | | UGUCUGCGCACUCGAAAAC |
| MG119-129 effector | 738 | MG119- | Nucleotide | Artificial | R0 (untruncated) | CUUAGUUACACAUCAUUAGAUGUGAGGGCAAUUAUAUGUCCUAAUGGUG [symbol] |
| sgRNA | | 129_sg2 | | sequence | | GGAUGCAGAUUAAAGCAUUAGGUUCAUGAACCAACAAUGUACUGUAAUC [symbol] |
| | | | | | | CUCGCUAAAUCGUGUAAGAAAUUACAUGGUACUAAAACA |
| MG119-129 effector | 739 | MG119- | Nucleotide | Artificial | R1 truncation | CUUAGUUACACAUCAUUAGAUGUGAGGGCAAUUAUAUGUCCUAAUGGUG [symbol] |
| sgRNA | | 129_sg2_1 | | sequence | | GGAUGCAGAUUAAAGCAUGUACUGUAAUCACUCGCUAAAUCGUGUAAGA [symbol] |
| | | | | | | AUUACAUGGUACUAAAACA |
| MG119-129 effector | 740 | MG119- | Nucleotide | Artificial | R1 truncation | CUUAGUUACACAUCAUUAGAUGUGAGGGCAAUUAUAUGUCCUAAUAAAU [symbol] |
| sgRNA | | 129_sg2_2 | | sequence | | GUGUAAGAAAUUACAUGGUACUAAAACA |
| MG119-129 effector | 741 | MG119- | Nucleotide | Artificial | R1 truncation | CUUAGUUACGAGGGCAAUUAUAUGUCCUAAUGGUGAGGAUGCAGAUUAAA |
| sgRNA | | 129_sg2_4 | | sequence | | GCAUUAGGUUCAUGAACCAACAAUGUACUGUAAUCACUCGCUAAAUCGUG |
| | | | | | | UAAGAAAUUACAUGGUACUAAAACA |
| MG119-129 effector | 742 | MG119- | Nucleotide | Artificial | R1 truncation | CUUAGUUACACAUCAUUAGAUGUGAUAAUGGUGAGGAUGCAGAUUAAAGC |
| sgRNA | | 129_sg2_5 | | sequence | | AUUAGGUUCAUGAACCAACAAUGUACUGUAAUCACUCGCUAAAUCGUGUA |
| | | | | | | AGAAAUUACAUGGUACUAAAACA |
| MG119-129 effector | 743 | MG119- | Nucleotide | Artificial | R1 truncation | CUUAGUUACACAUCAUUAGAUGUGAGGGCAAUUAUAUGUCCUAAUGGUGA |
| sgRNA | | 129_sg2_6 | | sequence | | GGAUGCAGAUUAAAGCAUUAGGUUCAUGAACCAACAAUGUACUGUAAUCA |
| | | | | | | CUCGCUAAAUACUAAAACA |
| MG119-129 effector | 744 | MG119- | Nucleotide | Artificial | R1 truncation | CUUAGUUACGAGGGCAAUUAUAUGUCCUAAUGGUGAGGAUGCAGAUUAAA |
| sgRNA | | 129_sg2_1.4 | | sequence | | GCAUGUACUGUAAUCACUCGCUAAAUCGUGUAAGAAAUUACAUGGUACUA |
| | | | | | | AAACA |
| MG119-129 effector | 745 | MG119- | Nucleotide | Artificial | R1 truncation | CUUAGUUACACAUCAUUAGAUGUGAGGGCAAUUAUAUGUCCUAAUGGUGA |
| sgRNA | | 129_sg2_1.6 | | sequence | | GGAUGCAGAUUAAAGCAUGUACUGUAAUCACUCGCUAAAUACUAAAACA |
| MG119-136 effector | 746 | MG119- | Nucleotide | Artificial | R1 truncation | AUUAUAAGUUAUAUUAUAAUUGGGCUAGUUGCAUAGCCUUUCGUGAGGU |
| sgRNA | | 136_sg1_1 | | sequence | | UGAAAAACACUCAUAAACUACUGUAAAUUGAAAAAUUGUAUGGUAGAUGC |
| | | | | | | AAC |
| MG119-136 effector | 747 | MG119- | Nucleotide | Artificial | R1 truncation | AUUAUAAGUUAUAUUAUAAUUGGGCUAGUUGCAUAGCCUUUCGUGAGGU |
| sgRNA | | 136_sg1_2 | | sequence | | UAACAAUAUAUGUUGAAAAACACUCAUAAACUACUGUAAAUUGAAAAAUU |
| | | | | | | GUAUGGUAGAUGCAAC |
| MG119-136 effector | 748 | MG119- | Nucleotide | Artificial | R1 truncation | AUUAUAAGUUAUAUUAUAAUUGGGCUAGUUGCAUAGCCUUUCGUGAUAA |
| sgRNA | | 136_sg1_3 | | sequence | | AUAUAUUAUCAUAAACUACUGUAAAUUGAAAAAUUGUAUGGUAGAUGCA |
| | | | | | | AC |
| MG119-136 effector | 749 | MG119- | Nucleotide | Artificial | R1 truncation | AUUAUAAGUUAUAUUAUAAUUGGGCUAGUUGCAUAGCCUUUCGUAAACUA |
| sgRNA | | 136_sg1_4 | | sequence | | CUGUAAAUUGAAAAAUUGUAUGGUAGAUGCAAC |
| MG119-136 effector | 750 | MG119- | Nucleotide | Artificial | R1 truncation | AUUAUAAGUUAUAUUAUAAUUGUUUCGUGAGGUUAACGAAUUAAAUAUA [symbol] |
| sgRNA | | 136_sg1_5 | | sequence | | UUAAUUCGUUGAAAAACACUCAUAAACUACUGUAAAUUGAAAAAUUGUA [symbol] |
| | | | | | | GGUAGAUGCAAC |
| MG119-136 effector | 751 | MG119- | Nucleotide | Artificial | R1 truncation | AUUGGGCUAGUUGCAUAGCCUUUCGUGAGGUUAACGAAUUAAAUAUAUU [symbol] |
| sgRNA | | 136_sg1_6 | | sequence | | AUUCGUUGAAAAACACUCAUAAACUACUGUAAAUUGAAAAAUUGUAUGG [symbol] |
| | | | | | | AGAUGCAAC |
| MG119-136 effector | 752 | MG119- | Nucleotide | Artificial | R1 truncation | AUUAUAAGUUAUAUUAUAAUUGGGCUAGUUGCAUAGCCUUUCGUGAGGU [symbol] |
| sgRNA | | 136_sg1_7 | | sequence | | UAACGAAUUAAAUAUAUUAAUUCGUUGAAAAACACUCAUAAACUAAUUG [symbol] |
| | | | | | | AAAAUUAGAUGCAAC |
| MG119-136 effector | 753 | MG119- | Nucleotide | Artificial | R1 truncation | AUUAUAAGUUAUAUUAUAAUUGGGCUAGUUGCAUAGCCUUUCGUGAGGU [symbol] |
| sgRNA | | 136_sg1_8 | | sequence | | UAACGAAUUAAAUAUAUUAAUUCGUUGAAAAACACUCAUAAACC |
| MG119-136 effector | 754 | MG119- | Nucleotide | Artificial | R1 truncation | AUUAUAAGUUAUAUUAUAAUUGGGCUAGUUGCAUAGCCUUUCGAACGAAU |
| sgRNA | | 136_sgl_9 | | sequence | | UAAAUAUAUUAAUUCGUUUAAACUACUGUAAAUUGAAAAAUUGUAUGGU |
| | | | | | | AGAUGCAAC |
| MG119-2 effector | 755 | MG119- | Nucleotide | Artificial | | UACACAUCAUUAGAUGUGAGGGCAAUUUUAUGUCCUAAUAUUGAGGGUAU |
| sgRNA | | 2_sg1_WT | | sequence | | UAGGUAACAACACUUUGUUUUAAUAGAACAGUGUCUAAUAACCACUCGAU |
| | | | | | | AAGACCAUAUAAAAGAAAUUUUGUAUGGUUAUAAAAC |
| MG119-27 effector | 756 | MG119- | Nucleotide | Artificial | | UUGAUAUAACGUAUAUGAAUUUUCUAUUCCCCUGUGGGGGAAGGGUGUGU |
| sgRNA | | 27_sg2_WT | | sequence | | UGGAACGCCUUGGUUUGAGGUUUGAAAUAGAAAAAAGAAAUACGGGAAU |
| | | | | | | GUAUUAUCGAUUUCAGGCACUCAAAAAAUUCACUUAAGAAAUUAAGUGAA |
| | | | | | | UAUCCAAC |
| MG119-64 effector | 757 | MG119- | Nucleotide | Artificial | | GAAUAGGAUUUAUCCUAUGGGGCAGUUGGAUGCCCUUAGCCUGAGGAAUU |
| sgRNA | | 64_sg1_WT | | sequence | | UAUUUCACUUGGGAAGUACCAUUCUUAUUGAAUGACAGCUAGUUAUGUUA |
| | | | | | | CUGAAAAGUAAAAUGUUUGGUAUAUCCAAC |
| MG119-121 effector | 758 | MG119- | Nucleotide | Artificial | | AUUGAGGGCGACUGUGCGUCCUAAGUGUGAGGGAAUUUACCAAGUGAAUA |
| sgRNA | | 121_sg1_W | | sequence | | UCCACUCAUAAAGUCUUGAAUAUAUUGAUAUUACAAGACAUCACCCUGCA |
| | | T | | | | AGUAGCAGGUAAAAAACUCUAGAAAUAGAGUUUUUUAAGUA |
| MG119-133 effector | 759 | MG119- | Nucleotide | Artificial | | UAAAUAAAUUAAAAAUACAUAUUAUAUACGGGUGAUUUACCAUCCGAAGG |
| sgRNA | | 133_sg1_W | | sequence | | UCGAGGGUGUGGGUUUAAUAUCUGGGUUUAGACCAAAAGGAUUUUCACAU |
| | | T | | | | CACUCGAUAAAUCAUAUGGUUGAAAAACUAUGUGAUAUGUAAAU |
| MG119-137 effector | 760 | MG119- | Nucleotide | Artificial | | UAAAUACCGAUAUAAAAUAUCGUGGCUGAUUCAGCUAGGGGAAACCUACA |
| sgRNA | | 137_sg1_W | | sequence | | CAUAUGUUAAGAGAAUAUAUGUGAGCCAAAGAGUGAGGGAUUUUCCACUC |
| | | T | | | | ACUAAAGCCUAUGUAGAAAUACAUGGGUACUGAAC |
| MG119-3 effector | 761 | MG119- | Nucleotide | Artificial | | UUAAAAUUUUAGAUAUGAUUUUUAUAUUCCCUUUAUGGGAAGGGUGUGU |
| sgRNA | | 3_sg1_WT | | sequence | | UGGAACGCCUUAGUUUGAGGUUUGAAUAUAAAAAGUGAAACAAAUGGAG |
| | | | | | | AUUUGUAACACCUUCAAACACUCAAAAAAUUCAUUUAGAAAUAAAUGAAU |
| | | | | | | AUCCAAC |
| MG119-97 effector | 762 | MG119- | Nucleotide | Artificial | | UCAUGGGGUGAUUUGGCAUCCUACAAUCGAGAUAGUGUAUAUAAGACUA [symbol] |
| sgRNA | | 97_sg1_WT | | sequence | | AUUAAUAUAGCAAUAAAAACACUCGAUAAGGCUCUAAGAAUUUUCCAACC |
| | | | | | | GAAUUCUUAACCACCUUGUCCGAAAAGGACAACAAAAUAUGAAAAUAUUU |
| | | | | | | UGAAAG |
| MG119-118 effector | 763 | MG119- | Nucleotide | Artificial | | CGGCAGGGCGACUUAGCGUCCAAAAGUCGAGUGAUUUGACGAUAAUAUG [symbol] |
| sgRNA | | 118_sg1_W | | sequence | | CAAACUUUAAUAGUAAUAGUUUACUCUCGAUAAGGUAACAAAUUUUUAG [symbol] |
| | | T | | | | UUUGUUGAAACUGCAUAUAUAAAAUCCAUAUGCACAUCUCGUAUUUGAA [symbol] |
| | | | | | | AGAUAUGAAAUGUAAGUUGA |
| MG119-128 effector | 764 | MG119- | Nucleotide | Artificial | | UUAGUUACACAUCAUUAGAUGUGAGGGCAAUUUUAUGUCCUAAUAUUGAG |
| sgRNA | | 128_sg2_W | | sequence | | GGUAUUAGGUAACAACACUUUGUUUUAAUAGAACAGUGUCUAAUAACCAC |
| | | T | | | | UCGAUAAGACCAUAUGAAAGUAUGGUUAUAAAACU |
| MG119-129 effector | 765 | MG119- | Nucleotide | Artificial | | UACACAUCAUUAGAUGUGAGGGCAAUUUUAUGUCCUAAUGGUGAGGAUGC |
| sgRNA | | 129_sg1_W | | sequence | | AGAUUAAAGCAUUAGGUUCAUGAACCAACAAUGUACUGUAAUCACUCGCU |
| | | T | | | | AAAUCGUGUAAGAAAUUACAUGGUACUAAAACA |
| MG119-136 effector | 766 | MG119- | Nucleotide | Artificial | | AUUAUAAGUUAUAUUAUAAUUGGGCUAGUUGCAUAGCCUUUCGUGAGGU |
| sgRNA | | 136_sg1_W | | sequence | | UAACGAAAAAAAUAUAUUUUUUUGUUGAAAAACACUCAUAAACUACUGUA |
| | | T | | | | AAUUGAAAAAUUGUAUGGUAGAUGCAAC |
| MG119-2 sgRNA | 767 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA A1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrUrUr |
| | | | | | | UrUrArCrArGrArUrArCrGrArA*mC*mC*mU |
| MG119-2 sgRNA | 768 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA B1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | ArCrArGrArUrArCrGrArArCrC*mU*mA*mA |
| MG119-2 sgRNA | 769 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA C1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArCrArGr |
| | | | | | | ArUrArCrGrArArCrCrUrArArA*mnC*mU*mU |
| MG119-2 sgRNA | 770 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA D1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArGrAr |
| | | | | | | UrArCrGrArArCrCrUrArArArC*mU*mU*mU |
| MG119-2 sgRNA | 771 | MG119-2 | Nucleotide | Artificial | | mU*mA*mC*rArCrArUrCrArUrUrArGrArUrGrUrGrArGrGrGrCrArArUrUrUrUrArU |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA E1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrArUr |
| | | | | | | ArCrGrArArCrCrUrArArArCrU*mU*mU*mC |
| MG119-2 sgRNA | 772 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA F1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArArAr |
| | | | | | | CrCrUrGrUrCrArGrUrGrArUrU*mnG*mG*mG |
| MG119-2 sgRNA | 773 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA G1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArArCr |
| | | | | | | CrUrGrUrCrArGrUrGrArUrUrG*mG*mG*mU |
| MG119-2 sgRNA | 774 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA H1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArCrCr |
| | | | | | | UrGrUrCrArGrUrGrArUrUrGrG*mG*mU*mU |
| MG119-2 sgRNA | 775 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA A2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrArUr |
| | | | | | | UrGrGrGrUrUrCrCrGrArArUrC*mC*mU*mC |
| MG119-2 sgRNA | 776 | MG119-2 | Nucleotide | Artificial | | mU*mA*C*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA B2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrUrCr |
| | | | | | | CrGrArArUrCrCrUrCrCrUrCrC*mU*mG*mA |
| MG119-2 sgRNA | 777 | MG119-2 | Nucleotide | Artificial | | mU*mA*mC*rArCrArUrCrArUrUrArGrArUrGrUrGrArGrGrGrCrArArUrUrUrUrArU |
| targeting TRAC | | hTRAC | | sequence | | GrUrCrCrUrArArUrArUrUrGrArGrGrGrUrArUrUrArGrGrUrArArCrArArCrArCrUrU |
| | | sgRNA C2 | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrGrArArU |
| | | | | | | CrCrUrCrCrUrCrCrUrGrArArA*mG*mU*mG |
| MG119-2 sgRNA | 778 | MG119-2 | Nucleotide | Artificial | | mU*mA*mC*rArCrArUrCrArUrUrArGrArUrGrUrGrArGrGrGrCrArArUrUrUrUrArU |
| targeting TRAC | | hTRAC | | sequence | | GrUrCrCrUrArArUrArUrUrGrArGrGrGrUrArUrUrArGrGrUrArArCrArArCrArCrUrU |
| | | sgRNA D2 | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrArArUrC |
| | | | | | | CrUrCrCrUrCrCrUrGrArArArG*mU*mG*mG |
| MG119-2 sgRNA | 779 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA E2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrCr |
| | | | | | | CrUrGrArArArGrUrGrGrCrCrG*mG*mG*mU |
| MG119-2 sgRNA | 780 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA F2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrGr |
| | | | | | | ArArArGrUrGrGrCrCrGrGrGrU*mU*mU*mA |
| MG119-2 sgRNA | 781 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA G2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArArAr |
| | | | | | | GrUrGrGrCrCrGrGrGrUrUrUrA*mA*mU*mC |
| MG119-2 sgRNA | 782 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA H2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrCrUr |
| | | | | | | GrCrUrCrArUrGrArCrGrCrUrG*mnC*mG*mG |
| MG119-2 sgRNA | 783 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA A3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrUrGr |
| | | | | | | CrUrCrArUrGrArCrGrCrUrGrC*mG*mnG*mC |
| MG119-2 sgRNA | 784 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA B3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrCrAr |
| | | | | | | UrGrArCrGrCrUrGrCrGrGrCrU*mG*mU*mG |
| MG119-2 sgRNA | 785 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA C3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArCrGr |
| | | | | | | CrUrGrCrGrGrCrUrGrUrGrGrU*mC*mC*mA |
| MG119-2 sgRNA | 786 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA D3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrCrUr |
| | | | | | | UrCrArArGrGrCrCrCrCrUrCrA*mC*mC*mU |
| MG119-2 sgRNA | 787 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA E3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrGrCr |
| | | | | | | CrCrCrUrCrArCrCrUrCrArGrC*mU*mG*mG |
| MG119-2 sgRNA | 788 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA F3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrCrCr |
| | | | | | | CrCrUrCrArCrCrUrCrArGrCrU*mG*mG*mA |
| MG119-2 sgRNA | 789 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA G3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrCrAr |
| | | | | | | GrCrUrGrGrArCrCrArCrArGrC*mC*mG*mC |
| MG119-2 sgRNA | 790 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA H3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrGr |
| | | | | | | ArCrCrArCrArGrCrCrGrCrArG*mC*mG*mU |
| MG119-2 sgRNA | 791 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA A4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArGrCr |
| | | | | | | ArGrArUrUrArArArCrCrCrGrG*mC*mC*mA |
| MG119-2 sgRNA | 792 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA B4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArCrCrCr |
| | | | | | | GrGrCrCrArCrUrUrUrCrArGrG*mA*mG*mG |
| MG119-2 sgRNA | 793 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA C4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrGrAr |
| | | | | | | GrGrArGrGrArUrUrCrGrGrArA*mC*mC*mC |
| MG119-2 sgRNA | 794 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA D4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrArGr |
| | | | | | | GrArGrGrArUrUrCrGrGrArArC*mC*mC*mA |
| MG119-2 sgRNA | 795 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA E4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArGrGr |
| | | | | | | ArGrGrArUrUrCrGrGrArArCrC*mC*mA*mA |
| MG119-2 sgRNA | 796 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA F4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArArCr |
| | | | | | | CrCrArArUrCrArCrUrGrArCrA*mG*mG*mU |
| MG119-2 sgRNA | 797 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA G4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArCrCr |
| | | | | | | CrArArUrCrArCrUrGrArCrArG*mG*mU*mU |
| MG119-2 sgRNA | 798 | MG119-2 | Nucleotide | Artificial | | mU*mA*mC*rArCrArUrCrArUrUrArGrArUrGrUrGrArGrGrGrCrArArUrUrUrUrArUr |
| targeting TRAC | | hTRAC | | sequence | | GrUrCrCrUrArArUrArUrUrGrArGrGrGrUrArUrUrArGrGrUrArArCrArArCrArCrUrUr |
| | | sgRNA H4 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrArCr |
| | | | | | | ArGrGrUrUrUrUrGrArArArGrU*mU*mU*mA |
| DNA Sequence of | 799 | MG119-2 | Nucleotide | Artificial | | GGTTTTACAGATACGAACCT |
| TRAC Target Site | | hTRAC | | sequence | | |
| | | Target Site | | | | |
| | | A1 | | | | |
| DNA Sequence of | 800 | MG119-2 | Nucleotide | Artificial | | TTTTACAGATACGAACCTAA |
| TRAC Target Site | | hTRAC | | sequence | | |
| | | Target Site | | | | |
| | | B1 | | | | |
| DNA Sequence of | 801 | MG119-2 | Nucleotide | Artificial | | ACAGATACGAACCTAAACTT |
| TRAC Target Site | | hTRAC | | sequence | | |
| | | Target Site | | | | |
| | | C1 | | | | |
| DNA Sequence of | 802 | MG119-2 | Nucleotide | Artificial | | CAGATACGAACCTAAACTTT |
| TRAC Target Site | | hTRAC | | sequence | | |
| | | Target Site | | | | |
| | | DI | | | | |
| DNA Sequence of | 803 | MG119-2 | Nucleotide | Artificial | | AGATACGAACCTAAACTTTC |
| TRAC Target Site | | hTRAC | | sequence | | |
| | | Target Site | | | | |
| | | E1 | | | | |
| DNA Sequence of | 804 | MG119-2 | Nucleotide | Artificial | | AAAACCTGTCAGTGATTGGG |
| TRAC Target Site | | hTRAC | | sequence | | |
| | | Target Site | | | | |
| | | F1 | | | | |
| DNA Sequence of | 805 | MG119-2 | Nucleotide | Artificial | | AAACCTGTCAGTGATTGGGT |
| TRAC Target Site | | hTRAC | | sequence | | |
| | | Target Site | | | | |
| | | G1 | | | | |
| DNA Sequence of | 806 | MG119-2 | Nucleotide | Artificial | | AACCTGTCAGTGATTGGGTT |
| TRAC Target Site | | hTRAC | | sequence | | |
| | | Target Site | | | | |
| | | H1 | | | | |
| DNA Sequence of | 807 | MG119-2 | Nucleotide | Artificial | | TGATTGGGTTCCGAATCCTC |
| TRAC Target Site | | hTRAC | | sequence | | |
| | | Target Site | | | | |
| | | A2 | | | | |
| DNA Sequence of | 808 | MG119-2 | Nucleotide | Artificial | | GTTCCGAATCCTCCTCCTGA |
| TRAC Target Site | | hTRAC | | sequence | | |
| | | Target Site | | | | |
| | | B2 | | | | |
| DNA Sequence of | 809 | MG119-2 | Nucleotide | Artificial | | GAATCCTCCTCCTGAAAGTG |
| TRAC Target Site | | hTRAC | | sequence | | |
| | | Target Site | | | | |
| | | C2 | | | | |
| DNA Sequence of | 810 | MG119-2 | Nucleotide | Artificial | | AATCCTCCTCCTGAAAGTGG |
| TRAC Target Site | | hTRAC | | sequence | | |
| | | Target Site | | | | |
| | | D2 | | | | |
| DNA Sequence of | 811 | MG119-2 | Nucleotide | Artificial | | CCTCCTGAAAGTGGCCGGGT |
| TRAC Target Site | | hTRAC | | sequence | | |
| | | Target Site | | | | |
| | | E2 | | | | |
| DNA Sequence of | 812 | MG119-2 | Nucleotide | Artificial | | CCTGAAAGTGGCCGGGTTTA |
| TRAC Target Site | | hTRAC | | sequence | | |
| | | Target Site | | | | |
| | | F2 | | | | |
| DNA Sequence of | 813 | MG119-2 | Nucleotide | Artificial | | GAAAGTGGCCGGGTTTAATC |
| TRAC Target Site | | hTRAC | | sequence | | |
| | | Target Site | | | | |
| | | G2 | | | | |
| DNA Sequence of | 814 | MG119-2 | Nucleotide | Artificial | | ATCTGCTCATGACGCTGCGG |
| TRAC Target Site | | hTRAC | | sequence | | |
| | | Target Site | | | | |
| | | H2 | | | | |
| DNA Sequence of | 815 | MG119-2 | Nucleotide | Artificial | | TCTGCTCATGACGCTGCGGC |
| TRAC Target Site | | hTRAC | | sequence | | |
| | | Target Site | | | | |
| | | A3 | | | | |
| DNA Sequence of | 816 | MG119-2 | Nucleotide | Artificial | | CTCATGACGCTGCGGCTGTG |
| TRAC Target Site | | hTRAC | | sequence | | |
| | | Target Site | | | | |
| | | B3 | | | | |
| DNA Sequence of | 817 | MG119-2 | Nucleotide | Artificial | | GACGCTGCGGCTGTGGTCCA |
| TRAC Target Site | | hTRAC | | sequence | | |
| | | Target Site | | | | |
| | | C3 | | | | |
| DNA Sequence of | 818 | MG119-2 | Nucleotide | Artificial | | AGCTTCAAGGCCCCTCACCT |
| TRAC Target Site | | hTRAC | | sequence | | |
| | | Target Site | | | | |
| | | D3 | | | | |
| DNA Sequence of | 819 | MG119-2 | Nucleotide | Artificial | | AGGCCCCTCACCTCAGCTGG |
| TRAC Target Site | | hTRAC | | sequence | | |
| | | Target Site | | | | |
| | | E3 | | | | |
| DNA Sequence of | 820 | MG119-2 | Nucleotide | Artificial | | GGCCCCTCACCTCAGCTGGA |
| TRAC Target Site | | hTRAC | | sequence | | |
| | | Target Site | | | | |
| | | F3 | | | | |
| DNA Sequence of | 821 | MG119-2 | Nucleotide | Artificial | | CTCAGCTGGACCACAGCCGC |
| TRAC Target Site | | hTRAC | | sequence | | |
| | | Target Site | | | | |
| | | G3 | | | | |
| DNA Sequence of | 822 | MG119-2 | Nucleotide | Artificial | | CTGGACCACAGCCGCAGCGT |
| TRAC Target Site | | hTRAC | | sequence | | |
| | | Target Site | | | | |
| | | H3 | | | | |
| DNA Sequence of | 823 | MG119-2 | Nucleotide | Artificial | | GAGCAGATTAAACCCGGCCA |
| TRAC Target Site | | hTRAC | | sequence | | |
| | | Target Site | | | | |
| | | A4 | | | | |
| DNA Sequence of | 824 | MG119-2 | Nucleotide | Artificial | | ACCCGGCCACTTTCAGGAGG |
| TRAC Target Site | | hTRAC | | sequence | | |
| | | Target Site | | | | |
| | | B4 | | | | |
| DNA Sequence of | 825 | MG119-2 | Nucleotide | Artificial | | AGGAGGAGGATTCGGAACCC |
| TRAC Target Site | | hTRAC | | sequence | | |
| | | Target Site | | | | |
| | | C4 | | | | |
| DNA Sequence of | 826 | MG119-2 | Nucleotide | Artificial | | GGAGGAGGATTCGGAACCCA |
| TRAC Target Site | | hTRAC | | sequence | | |
| | | Target Site | | | | |
| | | D4 | | | | |
| DNA Sequence of | 827 | MG119-2 | Nucleotide | Artificial | | GAGGAGGATTCGGAACCCAA |
| TRAC Target Site | | hTRAC | | sequence | | |
| | | Target Site | | | | |
| | | E4 | | | | |
| DNA Sequence of | 828 | MG119-2 | Nucleotide | Artificial | | GAACCCAATCACTGACAGGT |
| TRAC Target Site | | hTRAC | | sequence | | |
| | | Target Site | | | | |
| | | F4 | | | | |
| DNA Sequence of | 829 | MG119-2 | Nucleotide | Artificial | | AACCCAATCACTGACAGGTT |
| TRAC Target Site | | hTRAC | | sequence | | |
| | | Target Site | | | | |
| | | G4 | | | | |
| DNA Sequence of | 830 | MG119-2 | Nucleotide | Artificial | | TGACAGGTTTTGAAAGTTTA |
| TRAC Target Site | | hTRAC | | sequence | | |
| | | Target Site | | | | |
| | | H4 | | | | |
| MG119-125 sgRNA | 831 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A1 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrArCrUrGrGrGrUrGrUrUrGrArGrCrUrU*mC*mU*mU |
| MG119-125 sgRNA | 832 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B1 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrGrArGrArGrCrGrCrUrCrArGrGrArArG*mC*mU*mG |
| MG119-125 sgRNA | 833 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA C1 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrUrGrArArGrCrUrCrUrCrCrArGrCrArCrG*mG*mG*mC |
| MG119-125 sgRNA | 834 | MG119-125 | Nucleotide | Artificial | | mU*mA*mA*rArCrArArUrUrGrArArUrArArArArArArUrArUrUrArUrArUrArCrGrG |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA D1 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrGrCrArCrGrGrGrCrArGrCrArGrGrCrCrU*mU*mG*mG |
| MG119-125 sgRNA | 835 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA El | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrGrCrGrGrArGrGrUrCrCrUrCrGrArG*mnC*mnG*mC |
| MG119-125 sgRNA | 836 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F1 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrGrArGrCrGrCrGrGrGrCrUrUrGrGrCrC*mU*mU*mC |
| MG119-125 sgRNA | 837 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G1 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrUrCrGrCrUrGrArGrCrGrUrGrCrUrC*mA*mG*mA |
| MG119-125 sgRNA | 838 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H1 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrGrCrGrUrGrGrUrArCrUrCrGrGrCrC*mA*mG*mU |
| MG119-125 sgRNA | 839 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrUrCrUrGrGrCrGrCrCrGrCrCrGrUrUrC*mU*mC*mC |
| MG119-125 sgRNA | 840 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrGrUrUrCrUrCrCrUrUrGrArGrArGrC*mC*mU*mC |
| MG119-125 sgRNA | 841 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrUrCrUrCrCrUrUrGrArGrArGrCrCrUrC*mA*mA*mG |
| MG119-125 sgRNA | 842 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrArGrArGrCrCrUrCrArArGrGrCrGrC*mG*mnC*mG |
| MG119-125 sgRNA | 843 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrArArGrGrCrGrCrGrCrGrGrCrCrArArG*mC*mG*mC |
| MG119-125 sgRNA | 844 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrCrGrCrUrGrGrCrGrCrArGrCrUrCrG*mU*mC*mG |
| MG119-125 sgRNA | 845 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArUrGrCrGrUrGrCrGrCrArGrCrGrCrG*mU*mC*mC |
| MG119-125 sgRNA | 846 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H2 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrArUrGrGrGrCrGrCrGrCrGrCrGrCrGrG*mU*mC*mG |
| MG119-125 sgRNA | 847 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrGrCrCrCrArGrUrGrGrGrCrUrCrArGrC*mU*mU*mC |
| MG119-125 sgRNA | 848 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrUrGrGrGrCrUrCrArGrCrUrUrCrUrC*mU*mU*mG |
| MG119-125 sgRNA | 849 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrUrGrGrGrCrUrCrArGrCrUrUrCrUrCrU*mU*mnG*mC |
| MG119-125 sgRNA | 850 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrUrUrGrGrArGrCrUrCrUrGrCrGrCrG*mC*mA*mG |
| MG119-125 sgRNA | 851 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrUrUrGrGrArGrCrUrCrUrGrCrGrCrGrC*mA*mG*mC |
| MG119-125 sgRNA | 852 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrUrUrCrUrGrGrCrGrGrUrArGrArGrC*mU*mC*mC |
| MG119-125 sgRNA | 853 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G3 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrUrGrGrCrGrGrUrArGrArGrCrUrCrC*mA*mU*mC |
| MG119-125 sgRNA | 854 | MG119-125 | Nucleotide | Artificial | | mU*mA*mA*rArCrArArUrUrGrArArUrArArArArArArUrArUrUrArUrArUrArCrGrG |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA H3 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrUrCrUrCrCrUrCrCrUrGrCrCrArCrUrUrC*mU*mU*mC |
| MG119-125 sgRNA | 855 | MG119-125 | Nucleotide | Artificial | | mU*mA*mA*rArCrArArUrUrGrArArUrArArArArArArUrArUrUrArUrArUrArCrGrG |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA A4 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrCrCrUrGrCrCrArCrUrUrCrUrUrCrUrGrG*mA*mA*mG |
| MG119-125 sgRNA | 856 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrCrCrArCrUrUrCrUrUrCrUrGrGrArArG*mU*mnC*mG |
| MG119-125 sgRNA | 857 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrUrUrCrUrUrCrUrGrGrArArGrUrCrGrU*mC*mC*mA |
| MG119-125 sgRNA | 858 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrGrUrArGrGrGrCrUrGrCrArCrCrUrUrG*mG*mC*mC |
| MG119-125 sgRNA | 859 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrGrCrCrUrUrCrArCrCrUrCrCrUrCrC*mA*mG*InA |
| MG119-125 sgRNA | 860 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrArCrCrUrCrCrUrCrCrArGrArUrCrC*mU*mU*mnG |
| MG119-125 sgRNA | 861 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA G4 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrCrCrUrCrCrArGrArUrCrCrUrUrGrCrUrC*mA*mU*mC |
| MG119-125 sgRNA | 862 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H4 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrArGrArUrCrCrUrUrGrCrUrCrArUrC*mU*mC*mC |
| MG119-125 sgRNA | 863 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArUrCrCrUrUrGrCrUrCrArUrCrUrCrC*mU*mG*mC |
| MG119-125 sgRNA | 864 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrCrUrCrArUrCrUrCrCrUrGrCrCrUrC*mA*mG*mG |
| MG119-125 sgRNA | 865 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrCrCrUrCrArGrGrCrCrCrUrCrUrGrUrC*mU*mC*mC |
| MG119-125 sgRNA | 866 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrArGrGrCrCrCrUrCrUrGrUrCrUrCrCrU*mU*mU*mU |
| MG119-125 sgRNA | 867 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrUrGrUrCrUrCrCrUrUrUrUrCrCrArG*mG*U*mU |
| MG119-125 sgRNA | 868 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA F5 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrCrUrGrUrCrUrCrCrUrUrUrUrCrCrArGrG*mU*mU*mA |
| MG119-125 sgRNA | 869 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA G5 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrUrUrUrCrCrArGrGrUrUrArUrCrCrCrArG*mA*mA*mC |
| MG119-125 sgRNA | 870 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H5 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrGrUrUrArUrCrCrCrArGrArArCrUrCrC*mU*mG*mG |
| MG119-125 sgRNA | 871 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrArArCrUrCrCrUrGrGrGrUrCrArCrA*mG*mG*mG |
| MG119-125 sgRNA | 872 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArArCrUrCrCrUrGrGrGrUrCrArCrArG*mG*mG*mC |
| MG119-125 sgRNA | 873 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrGrGrUrCrArCrArGrGrGrCrCrGrArGrC*mU*mG*mU |
| MG119-125 sgRNA | 874 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrCrUrGrUrUrCrGrCrGrCrArGrCrUrU*mG*mnC*mU |
| MG119-125 sgRNA | 875 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrUrUrGrUrCrArArGrGrArGrCrUrUrU*mA*mG*mG |
| MG119-125 sgRNA | 876 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrUrCrUrGrUrCrUrCrArCrCrCrUrCrCrA*mG*mC*mC |
| MG119-125 sgRNA | 877 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrUrGrUrCrUrCrArCrCrCrUrCrCrArG*mC*mC*mU |
| MG119-125 sgRNA | 878 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H6 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrCrArGrCrCrUrArArArGrCrUrCrCrU*mU*mG*mA |
| MG119-125 sgRNA | 879 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrArGrCrCrUrArArArGrCrUrCrCrUrU*mG*mA*mC |
| MG119-125 sgRNA | 880 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrCrCrUrArArArGrCrUrCrCrUrUrGrArC*mA*mA*mC |
| MG119-125 sgRNA | 881 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArArArGrCrUrCrCrUrUrGrArCrArArCrU*mG*mG*mG |
| MG119-125 sgRNA | 882 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA D7 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrUrGrArCrArArCrUrGrGrGrArCrArGrCrG*mU*mG*mA |
| MG119-125 sgRNA | 883 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrArCrCrUrUrCrArGrCrArArGrCrUrG*mC*mG*mC |
| MG119-125 sgRNA | 884 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrUrUrCrArGrCrArArGrCrUrGrCrGrC*mG*mA*mA |
| MG119-125 sgRNA | 885 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrCrArGrCrArArGrCrUrGrCrGrCrGrArA*mC*mA*mG |
| MG119-125 sgRNA | 886 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H7 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrGrUrGrArCrCrCrArGrGrArGrUrUrCrU*mG*mG*mG |
| MG119-125 sgRNA | 887 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrUrGrArCrCrCrArGrGrArGrUrUrCrUrG*mG*mG*mA |
| MG119-125 sgRNA | 888 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrGrArGrUrUrCrUrGrGrGrArUrArArC*mC*mU*mG |
| MG119-125 sgRNA | 889 | MG119-125 | Nucleotide | Artificial | | mU*mA*mA*rArCrArArUrUrGrArArUrArArArArArArUrArUrUrArUrArUrArCrGrG |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA C8 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrGrGrArGrUrUrCrUrGrGrGrArUrArArCrC*mU*mG*mG |
| MG119-125 sgRNA | 890 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrGrArArArArGrGrArGrArCrArGrArGrG*mG*mC*mC |
| MG119-125 sgRNA | 891 | MG119-125 | Nucleotide | Artificial | | mU*mA*mA*rArCrArArUrUrGrArArUrArArArArArArUrArUrUrArUrArUrArCrGrGr |
| targeting APOAl | | hAPOA1 | | sequence | | GrCrGrArUrUrCrArCrCrGrUrCrCrGrArArGrGrUrUrGrArGrGrArUrArCrGrUrArArGr |
| | | sgRNA E8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArGrGrCrArGrGrArGrArUrGrArGrCrA*mA*mG*mG |
| MG119-125 sgRNA | 892 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrGrUrGrCrArGrCrCrCrUrArCrCrUrG*mG*mA*mC |
| MG119-125 sgRNA | 893 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrUrArCrCrUrGrGrArCrGrArCrUrUrCrCrA*mG*mA*mA |
| MG119-125 sgRNA | 894 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H8 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArCrCrUrGrGrArCrGrArCrUrUrCrCrArG*mA*mA*mG |
| MG119-125 sgRNA | 895 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA A9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrGrArCrGrArCrUrUrCrCrArGrArArGrA*mA*mG*mU |
| MG119-125 sgRNA | 896 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArArGrArArGrUrGrGrCrArGrGrArGrG*mA*mG*mA |
| MG119-125 sgRNA | 897 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA C9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrCrArGrArArGrGrUrGrGrArGrCrCrGrC*mU*mG*mC |
| MG119-125 sgRNA | 898 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA D9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArArGrGrUrGrGrArGrCrCrGrCrUrGrC*mnG*mC*mG |
| MG119-125 sgRNA | 899 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA E9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrUrGrCrGrCrGrCrArGrArGrCrUrCrCrA*mA*mG*mA |
| MG119-125 sgRNA | 900 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA F9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArGrArGrGrGrCrGrCrGrCrGrCrCrArGrA*mA*mG*mC |
| MG119-125 sgRNA | 901 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA G9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrGrArArGrCrUrGrCrArCrGrArGrCrUrGrC*mA*mA*mG |
| MG119-125 sgRNA | 902 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA H9 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrArCrUrGrGrGrCrGrArGrGrArGrArUrGrC*mG*mC*mG |
| MG119-125 sgRNA | 903 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArG |
| | | sgRNA A10 | | | | ArArUrArUrArArGrUrCrUrArGrGrCrUrUrUrGrGrCrCrArArCrArGrArUrUrUrCrCrG |
| | | | | | | UrArUrCrArCrUrCrGrArUrArArArUrCrArCrArUrArGrUrGrArArArArCrUrArUrGrU |
| | | | | | | GrGrUrArUrGrUrGrArArUrCrUrGrGrGrCrGrArGrGrArGrArUrGrCrG*mC*mG*mA |
| MG119-125 sgRNA | 904 | MG119-125 | Nucleotide | Artificial | | mU*mA*mA*rArCrArArUrUrGrArArUrArArArArArArUrArUrUrArUrArUrArCrGrGr |
| targeting APOA1 | | hAPOA1 | | sequence | | GrCrGrArUrUrCrArCrCrGrUrCrCrGrArArGrGrUrUrGrArGrGrArUrArCrGrUrArArGr |
| | | sgRNA B10 | | | | ArArUrArUrArArGrUrCrUrArGrGrCrUrUrUrGrGrCrCrArArCrArGrArUrUrUrCrCrGr |
| | | | | | | UrArUrCrArCrUrCrGrArUrArArArUrCrArCrArUrArGrUrGrArArArArCrUrArUrGrUr |
| | | | | | | GrGrUrArUrGrUrGrArArUrCrGrCrGrCrGrCrGrCrCrCrArUrGrUrGrG*mA*mC*mG |
| DNA Sequence of | 905 | MG119-125 | Nucleotide | Artificial | | CACTGGGTGTTGAGCTTCTT |
| APOA1 Target Site | | hAPOA1 | | sequence | | |
| | | Target Site | | | | |
| | | A1 | | | | |
| DNA Sequence of | 906 | MG119-125 | Nucleotide | Artificial | | CGAGAGCGCTCAGGAAGCTG |
| APOA1 Target Site | | hAPOA1 | | sequence | | |
| | | Target Site | | | | |
| | | B1 | | | | |
| DNA Sequence of | 907 | MG119-125 | Nucleotide | Artificial | | TGAAGCTCTCCAGCACGGGC |
| APOA1 Target Site | | hAPOA1 | | sequence | | |
| | | Target Site | | | | |
| | | C1 | | | | |
| DNA Sequence of | 908 | MG119-125 | Nucleotide | Artificial | | GCACGGGCAGCAGGCCTTGG |
| APOA1 Target Site | | hAPOA1 | | sequence | | |
| | | Target Site | | | | |
| | | D1 | | | | |
| DNA Sequence of | 909 | MG119-125 | Nucleotide | Artificial | | TGGCGGAGGTCCTCGAGCGC |
| APOA1 Target Site | | hAPOA1 | | sequence | | |
| | | Target Site | | | | |
| | | E1 | | | | |
| DNA Sequence of | 910 | MG119-125 | Nucleotide | Artificial | | CGAGCGCGGGCTTGGCCTTC |
| APOA1 Target Site | | hAPOA1 | | sequence | | |
| | | Target Site | | | | |
| | | F1 | | | | |
| DNA Sequence of | 911 | MG119-125 | Nucleotide | Artificial | | TCTCGCTGAGCGTGCTCAGA |
| APOA1 Target Site | | hAPOA1 | | sequence | | |
| | | Target Site | | | | |
| | | G1 | | | | |
| DNA Sequence of | 912 | MG119-125 | Nucleotide | Artificial | | TGGCGTGGTACTCGGCCAGT |
| APOA1 Target Site | | hAPOA1 | | sequence | | |
| | | Target Site | | | | |
| | | H1 | | | | |
| DNA Sequence of | 913 | MG119-125 | Nucleotide | Artificial | | GTCTGGCGCCGCCGTTCTCC |
| APOA1 Target Site | | hAPOA1 | | sequence | | |
| | | Target Site | | | | |
| | | A2 | | | | |
| DNA Sequence of | 914 | MG119-125 | Nucleotide | Artificial | | CCGTTCTCCTTGAGAGCCTC |
| APOA1 Target Site | | hAPOA1 | | sequence | | |
| | | Target Site | | | | |
| | | B2 | | | | |
| DNA Sequence of | 915 | MG119-125 | Nucleotide | Artificial | | TTCTCCTTGAGAGCCTCAAG |
| APOA1 Target Site | | hAPOAl | | sequence | | |
| | | Target Site | | | | |
| | | C2 | | | | |
| DNA Sequence of | 916 | MG119-125 | Nucleotide | Artificial | | TGAGAGCCTCAAGGCGCGCG |
| APOA1 Target Site | | hAPOA1 | | sequence | | |
| | | Target Site | | | | |
| | | D2 | | | | |
| DNA Sequence of | 917 | MG119-125 | Nucleotide | Artificial | | CAAGGCGCGCGGCCAAGCGC |
| APOA1 Target Site | | hAPOA1 | | sequence | | |
| | | Target Site | | | | |
| | | E2 | | | | |
| DNA Sequence of | 918 | MG119-125 | Nucleotide | Artificial | | AGCGCTGGCGCAGCTCGTCG |
| APOA1 Target Site | | hAPOA1 | | sequence | | |
| | | Target Site | | | | |
| | | F2 | | | | |
| DNA Sequence of | 919 | MG119-125 | Nucleotide | Artificial | | GATGCGTGCGCAGCGCGTCC |
| APOA1 Target Site | | hAPOA1 | | sequence | | |
| | | Target Site | | | | |
| | | G2 | | | | |
| DNA Sequence of | 920 | MG119-125 | Nucleotide | Artificial | | CATGGGCGCGCGCGCGGTCG |
| APOA1 Target Site | | hAPOAl | | sequence | | |
| | | Target Site | | | | |
| | | H2 | | | | |
| DNA Sequence of | 921 | MG119-125 | Nucleotide | Artificial | | CGCCCAGTGGGCTCAGCTTC |
| APOA1 Target Site | | hAPOA1 | | sequence | | |
| | | Target Site | | | | |
| | | A3 | | | | |
| DNA Sequence of | 922 | MG119-125 | Nucleotide | Artificial | | AGTGGGCTCAGCTTCTCTTG |
| APOA1 Target Site | | hAPOA1 | | sequence | | |
| | | Target Site | | | | |
| | | B3 | | | | |
| DNA Sequence of | 923 | MG119-125 | Nucleotide | Artificial | | GTGGGCTCAGCTTCTCTTGC |
| APOA1 Target Site | | hAPOA1 | | sequence | | |
| | | Target Site | | | | |
| | | C3 | | | | |
| DNA Sequence of | 924 | MG119-125 | Nucleotide | Artificial | | TCTTGGAGCTCTGCGCGCAG |
| APOA1 Target Site | | hAPOA1 | | sequence | | |
| | | Target Site | | | | |
| | | D3 | | | | |
| DNA Sequence of | 925 | MG119-125 | Nucleotide | Artificial | | CTTGGAGCTCTGCGCGCAGC |
| APOA1 Target Site | | hAPOA1 | | sequence | | |
| | | Target Site | | | | |
| | | E3 | | | | |
| DNA Sequence of | 926 | MG119-125 | Nucleotide | Artificial | | CCTTCTGGCGGTAGAGCTCC |
| APOA1 Target Site | | hAPOA1 | | sequence | | |
| | | Target Site | | | | |
| | | F3 | | | | |
| DNA Sequence of | 927 | MG119-125 | Nucleotide | Artificial | | TCTGGCGGTAGAGCTCCATC |
| APOA1 Target Site | | hAPOA1 | | sequence | | |
| | | Target Site | | | | |
| | | G3 | | | | |
| DNA Sequence of | 928 | MG119-125 | Nucleotide | Artificial | | TCTCCTCCTGCCACTTCTTC |
| APOA1 Target Site | | hAPOA1 | | sequence | | |
| | | Target Site | | | | |
| | | H3 | | | | |
| DNA Sequence of | 929 | MG119-125 | Nucleotide | Artificial | | CCTGCCACTTCTTCTGGAAG |
| APOA1 Target Site | | hAPOA1 | | sequence | | |
| | | Target Site | | | | |
| | | A4 | | | | |
| DNA Sequence of | 930 | MG119-125 | Nucleotide | Artificial | | GCCACTTCTTCTGGAAGTCG |
| APOA1 Target Site | | hAPOA1 | | sequence | | |
| | | Target Site | | | | |
| | | B4 | | | | |
| DNA Sequence of | 931 | MG119-125 | Nucleotide | Artificial | | CTTCTTCTGGAAGTCGTCCA |
| APOA1 Target Site | | hAPOA1 | | sequence | | |
| | | Target Site | | | | |
| | | C4 | | | | |
| DNA Sequence of | 932 | MG119-125 | Nucleotide | Artificial | | GGTAGGGCTGCACCTTGGCC |
| APOA1 Target Site | | hAPOA1 | | sequence | | |
| | | Target Site | | | | |
| | | D4 | | | | |
| DNA Sequence of | 933 | MG119-125 | Nucleotide | Artificial | | TGGCCTTCACCTCCTCCAGA |
| APOA1 Target Site | | hAPOA1 | | sequence | | |
| | | Target Site | | | | |
| | | E4 | | | | |
| DNA Sequence of | 934 | MG119-125 | Nucleotide | Artificial | | TCACCTCCTCCAGATCCTTG |
| APOA1 Target Site | | hAPOA1 | | sequence | | |
| | | Target Site | | | | |
| | | F4 | | | | |
| DNA Sequence of | 935 | MG119-125 | Nucleotide | Artificial | | CCTCCAGATCCTTGCTCATC |
| APOA1 Target Site | | hAPOA1 | | sequence | | |
| | | Target Site | | | | |
| | | G4 | | | | |
| DNA Sequence of | 936 | MG119-125 | Nucleotide | Artificial | | CCAGATCCTTGCTCATCTCC |
| APOA1 Target Site | | hAPOAl | | sequence | | |
| | | Target Site | | | | |
| | | H4 | | | | |
| DNA Sequence of | 937 | MG119-125 | Nucleotide | Artificial | | GATCCTTGCTCATCTCCTGC |
| APOA1 Target Site | | hAPOA1 | | sequence | | |
| | | Target Site | | | | |
| | | A5 | | | | |
| DNA Sequence of | 938 | MG119-125 | Nucleotide | Artificial | | TGCTCATCTCCTGCCTCAGG |
| APOA1 Target Site | | hAPOA1 | | sequence | | |
| | | Target Site | | | | |
| | | B5 | | | | |
| DNA Sequence of | 939 | MG119-125 | Nucleotide | Artificial | | GCCTCAGGCCCTCTGTCTCC |
| APOA1 Target Site | | hAPOA1 | | sequence | | |
| | | Target Site | | | | |
| | | C5 | | | | |
| DNA Sequence of | 940 | MG119-125 | Nucleotide | Artificial | | CAGGCCCTCTGTCTCCTTTT |
| APOA1 Target Site | | hAPOA1 | | sequence | | |
| | | Target Site | | | | |
| | | D5 | | | | |
| DNA Sequence of | 941 | MG119-125 | Nucleotide | Artificial | | TCTGTCTCCTTTTCCAGGTT |
| APOA1 Target Site | | hAPOA1 | | sequence | | |
| | | Target Site | | | | |
| | | E5 | | | | |
| DNA Sequence of | 942 | MG119-125 | Nucleotide | Artificial | | CTGTCTCCTTTTCCAGGTTA |
| APOA1 Target Site | | hAPOA1 | | sequence | | |
| | | Target Site | | | | |
| | | F5 | | | | |
| DNA Sequence of | 943 | MG119-125 | Nucleotide | Artificial | | TTTCCAGGTTATCCCAGAAC |
| APOA1 Target Site | | hAPOA1 | | sequence | | |
| | | Target Site | | | | |
| | | G5 | | | | |
| DNA Sequence of | 944 | MG119-125 | Nucleotide | Artificial | | GGTTATCCCAGAACTCCTGG |
| APOA1 Target Site | | hAPOA1 | | sequence | | |
| | | Target Site | | | | |
| | | H5 | | | | |
| DNA Sequence of | 945 | MG119-125 | Nucleotide | Artificial | | AGAACTCCTGGGTCACAGGG |
| APOA1 Target Site | | hAPOA1 | | sequence | | |
| | | Target Site | | | | |
| | | A6 | | | | |
| DNA Sequence of | 946 | MG119-125 | Nucleotide | Artificial | | GAACTCCTGGGTCACAGGGC |
| APOA1 Target Site | | hAPOA1 | | sequence | | |
| | | Target Site | | | | |
| | | B6 | | | | |
| DNA Sequence of | 947 | MG119-125 | Nucleotide | Artificial | | GGGTCACAGGGCCGAGCTGT |
| APOA1 Target Site | | hAPOA1 | | sequence | | |
| | | Target Site | | | | |
| | | C6 | | | | |
| DNA Sequence of | 948 | MG119-125 | Nucleotide | Artificial | | AGCTGTTCGCGCAGCTTGCT |
| APOA1 Target Site | | hAPOA1 | | sequence | | |
| | | Target Site | | | | |
| | | D6 | | | | |
| DNA Sequence of | 949 | MG119-125 | Nucleotide | Artificial | | AGTTGTCAAGGAGCTTTAGG |
| APOA1 Target Site | | hAPOA1 | | sequence | | |
| | | Target Site | | | | |
| | | E6 | | | | |
| DNA Sequence of | 950 | MG119-125 | Nucleotide | Artificial | | TTCTGTCTCACCCTCCAGCC |
| APOA1 Target Site | | hAPOA1 | | sequence | | |
| | | Target Site | | | | |
| | | F6 | | | | |
| DNA Sequence of | 951 | MG119-125 | Nucleotide | Artificial | | TCTGTCTCACCCTCCAGCCT |
| APOA1 Target Site | | hAPOA1 | | sequence | | |
| | | Target Site | | | | |
| | | G6 | | | | |
| DNA Sequence of | 952 | MG119-125 | Nucleotide | Artificial | | TCCAGCCTAAAGCTCCTTGA |
| APOA1 Target Site | | hAPOA1 | | sequence | | |
| | | Target Site | | | | |
| | | H6 | | | | |
| DNA Sequence of | 953 | MG119-125 | Nucleotide | Artificial | | CCAGCCTAAAGCTCCTTGAC |
| APOA1 Target Site | | hAPOA1 | | sequence | | |
| | | Target Site | | | | |
| | | A7 | | | | |
| DNA Sequence of | 954 | MG119-125 | Nucleotide | Artificial | | GCCTAAAGCTCCTTGACAAC |
| APOA1 Target Site | | hAPOA1 | | sequence | | |
| | | Target Site | | | | |
| | | B7 | | | | |
| DNA Sequence of | 955 | MG119-125 | Nucleotide | Artificial | | AAAGCTCCTTGACAACTGGG |
| APOA1 Target Site | | hAPOA1 | | sequence | | |
| | | Target Site | | | | |
| | | C7 | | | | |
| DNA Sequence of | 956 | MG119-125 | Nucleotide | Artificial | | TGACAACTGGGACAGCGTGA |
| APOA1 Target Site | | hAPOA1 | | sequence | | |
| | | Target Site | | | | |
| | | D7 | | | | |
| DNA Sequence of | 957 | MG119-125 | Nucleotide | Artificial | | CCACCTTCAGCAAGCTGCGC |
| APOA1 Target Site | | hAPOA1 | | sequence | | |
| | | Target Site | | | | |
| | | E7 | | | | |
| DNA Sequence of | 958 | MG119-125 | Nucleotide | Artificial | | CCTTCAGCAAGCTGCGCGAA |
| APOA1 Target Site | | hAPOA1 | | sequence | | |
| | | Target Site | | | | |
| | | F7 | | | | |
| DNA Sequence of | 959 | MG119-125 | Nucleotide | Artificial | | TCAGCAAGCTGCGCGAACAG |
| APOA1 Target Site | | hAPOA1 | | sequence | | |
| | | Target Site | | | | |
| | | G7 | | | | |
| DNA Sequence of | 960 | MG119-125 | Nucleotide | Artificial | | TGTGACCCAGGAGTTCTGGG |
| APOA1 Target Site | | hAPOA1 | | sequence | | |
| | | Target Site | | | | |
| | | H7 | | | | |
| DNA Sequence of | 961 | MG119-125 | Nucleotide | Artificial | | GTGACCCAGGAGTTCTGGGA |
| APOA1 Target Site | | hAPOA1 | | sequence | | |
| | | Target Site | | | | |
| | | A8 | | | | |
| DNA Sequence of | 962 | MG119-125 | Nucleotide | Artificial | | AGGAGTTCTGGGATAACCTG |
| APOA1 Target Site | | hAPOA1 | | sequence | | |
| | | Target Site | | | | |
| | | B8 | | | | |
| DNA Sequence of | 963 | MG119-125 | Nucleotide | Artificial | | GGAGTTCTGGGATAACCTGG |
| APOA1 Target Site | | hAPOA1 | | sequence | | |
| | | Target Site | | | | |
| | | C8 | | | | |
| DNA Sequence of | 964 | MG119-125 | Nucleotide | Artificial | | GGAAAAGGAGACAGAGGGCC |
| APOA1 Target Site | | hAPOA1 | | sequence | | |
| | | Target Site | | | | |
| | | D8 | | | | |
| DNA Sequence of | 965 | MG119-125 | Nucleotide | Artificial | | GAGGCAGGAGATGAGCAAGG |
| APOA1 Target Site | | hAPOA1 | | sequence | | |
| | | Target Site | | | | |
| | | E8 | | | | |
| DNA Sequence of | 966 | MG119-125 | Nucleotide | Artificial | | AGGTGCAGCCCTACCTGGAC |
| APOA1 Target Site | | hAPOA1 | | sequence | | |
| | | Target Site | | | | |
| | | F8 | | | | |
| DNA Sequence of | 967 | MG119-125 | Nucleotide | Artificial | | TACCTGGACGACTTCCAGAA |
| APOA1 Target Site | | hAPOA1 | | sequence | | |
| | | Target Site | | | | |
| | | G8 | | | | |
| DNA Sequence of | 968 | MG119-125 | Nucleotide | Artificial | | ACCTGGACGACTTCCAGAAG |
| APOA1 Target Site | | hAPOA1 | | sequence | | |
| | | Target Site | | | | |
| | | H8 | | | | |
| DNA Sequence of | 969 | MG119-125 | Nucleotide | Artificial | | GGACGACTTCCAGAAGAAGT |
| APOA1 Target Site | | hAPOA1 | | sequence | | |
| | | Target Site | | | | |
| | | A9 | | | | |
| DNA Sequence of | 970 | MG119-125 | Nucleotide | Artificial | | GAAGAAGTGGCAGGAGGAGA |
| APOA1 Target Site | | hAPOA1 | | sequence | | |
| | | Target Site | | | | |
| | | B9 | | | | |
| DNA Sequence of | 971 | MG119-125 | Nucleotide | Artificial | | CCAGAAGGTGGAGCCGCTGC |
| APOA1 Target Site | | hAPOA1 | | sequence | | |
| | | Target Site | | | | |
| | | C9 | | | | |
| DNA Sequence of | 972 | MG119-125 | Nucleotide | Artificial | | GAAGGTGGAGCCGCTGCGCG |
| APOA1 Target Site | | hAPOA1 | | sequence | | |
| | | Target Site | | | | |
| | | D9 | | | | |
| DNA Sequence of | 973 | MG119-125 | Nucleotide | Artificial | | CTGCGCGCAGAGCTCCAAGA |
| APOA1 Target Site | | hAPOA1 | | sequence | | |
| | | Target Site | | | | |
| | | E9 | | | | |
| DNA Sequence of | 974 | MG119-125 | Nucleotide | Artificial | | AGAGGGCGCGCGCCAGAAGC |
| APOA1 Target Site | | hAPOA1 | | sequence | | |
| | | Target Site | | | | |
| | | F9 | | | | |
| DNA Sequence of | 975 | MG119-125 | Nucleotide | Artificial | | GAAGCTGCACGAGCTGCAAG |
| APOA1 Target Site | | hAPOA1 | | sequence | | |
| | | Target Site | | | | |
| | | G9 | | | | |
| DNA Sequence of | 976 | MG119-125 | Nucleotide | Artificial | | ACTGGGCGAGGAGATGCGCG |
| APOA1 Target Site | | hAPOA1 | | sequence | | |
| | | Target Site | | | | |
| | | H9 | | | | |
| DNA Sequence of | 977 | MG119-125 | Nucleotide | Artificial | | CTGGGCGAGGAGATGCGCGA |
| APOA1 Target Site | | hAPOA1 | | sequence | | |
| | | Target Site | | | | |
| | | A10 | | | | |
| DNA Sequence of | 978 | MG119-125 | Nucleotide | Artificial | | CGCGCGCGCCCATGTGGACG |
| APOA1 Target Site | | hAPOA1 | | sequence | | |
| | | Target Site | | | | |
| | | B10 | | | | |
| MG119-129 sgRNA | 979 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA A1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArArGrCrUrCrUrCrCrArGrCrArCrGrG*mG*mC*mA |
| MG119-129 sgRNA | 980 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA B1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrGrCrGrGrArGrGrUrCrCrUrCrGrArGrC*mG*mC*mG |
| MG119-129 sgRNA | 981 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrUrCrGrCrUrGrArGrCrGrUrGrCrUrCrA*mG*mA*mU |
| MG119-129 sgRNA | 982 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA DI | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrA |
| | | | | | | GrGrCrGrUrGrGrUrArCrUrCrGrGrCrCrA*mG*mU*mC |
| MG119-129 sgRNA | 983 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA E1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | UrCrUrCrCrUrUrGrArGrArGrCrCrUrCrA*mA*mG*mG |
| MG119-129 sgRNA | 984 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA F1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArGrArGrCrCrUrCrArArGrGrCrGrCrG*mC*mG*mG |
| MG119-129 sgRNA | 985 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA G1 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrGrCrUrGrGrCrGrCrArGrCrUrCrGrU*mC*mG*mC |
| MG119-129 sgRNA | 986 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA HI | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrUrGrGrGrCrUrCrArGrCrUrUrCrUrCrU*mU*mG*mC |
| MG119-129 sgRNA | 987 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA A2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrUrUrGrGrArGrCrUrCrUrGrCrGrCrGrC*mA*mG*mC |
| MG119-129 sgRNA | 988 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA B2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrUrGrGrCrGrGrUrArGrArGrCrUrCrCrA*mU*mC*mU |
| MG119-129 sgRNA | 989 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrA |
| | | | | | | CrUrCrCrUrCrCrUrGrCrCrArCrUrUrCrU*mU*mC*mU |
| MG119-129 sgRNA | 990 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA D2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrGrCrCrUrUrCrArCrCrUrCrCrUrCrCrA*mG*mA*mU |
| MG119-129 sgRNA | 991 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA E2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrArCrCrUrCrCrUrCrCrArGrArUrCrCrU*mU*mG*mC |
| MG119-129 sgRNA | 992 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA F2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrUrCrArUrCrUrCrCrUrGrCrCrUrCrA*mG*mG*mC |
| MG119-129 sgRNA | 993 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA G2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrUrGrUrCrUrCrCrUrUrUrUrCrCrArGrG*mU*mU*mA |
| MG119-129 sgRNA | 994 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA H2 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | UrUrCrCrArGrGrUrUrArUrCrCrCrArGrA*mA*mC*mU |
| MG119-129 sgRNA | 995 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA A3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArArCrUrCrCrUrGrGrGrUrCrArCrArG*mG*mG*mC |
| MG119-129 sgRNA | 996 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA B3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrUrGrUrUrCrGrCrGrCrArGrCrUrUrG*mC*mU*mG |
| MG119-129 sgRNA | 997 | MG119-129 | Nucleotide | Artificial | | mU*mA*mC*ArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrUrUrGrUrCrArArGrGrArGrCrUrUrUrA*mG*mG*mC |
| MG119-129 sgRNA | 998 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA D3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | UrCrUrGrUrCrUrCrArCrCrCrUrCrCrArG*mC*mC*mU |
| MG119-129 sgRNA | 999 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA E3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrUrGrUrCrUrCrArCrCrCrUrCrCrArGrC*mC*mU*mA |
| MG119-129 sgRNA | 1000 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA F3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrCrArGrCrCrUrArArArGrCrUrCrCrUrU*mG*mA*mC |
| MG119-129 sgRNA | 1001 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA G3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | ArArGrCrUrCrCrUrUrGrArCrArArCrUrG*mG*mG*mA |
| MG119-129 sgRNA | 1002 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA H3 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArCrArArCrUrGrGrGrArCrArGrCrGrU*mG*mA*mC |
| MG119-129 sgRNA | 1003 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA A4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrA |
| | | | | | | CrArGrCrArArGrCrUrGrCrGrCrGrArArC*mA*mG*mC |
| MG119-129 sgRNA | 1004 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA B4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrUrGrArCrCrCrArGrGrArGrUrUrCrUrG*mG*mG*mA |
| MG119-129 sgRNA | 1005 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrGrArGrUrUrCrUrGrGrGrArUrArArCrC*mU*mG*mG |
| MG119-129 sgRNA | 1006 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA D4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrGrUrGrCrArGrCrCrCrUrArCrCrUrGrG*mA*mC*mG |
| MG119-129 sgRNA | 1007 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA E4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | ArCrCrUrGrGrArCrGrArCrUrUrCrCrArG*mA*mA*mG |
| MG119-129 sgRNA | 1008 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA F4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrCrUrGrGrArCrGrArCrUrUrCrCrArGrA*mA*mG*mA |
| MG119-129 sgRNA | 1009 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA G4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArGrGrGrCrGrCrGrCrGrCrCrArGrArA*mG*mC*mU |
| MG119-129 sgRNA | 1010 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA H4 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrA |
| | | | | | | CrUrGrGrGrCrGrArGrGrArGrArUrGrCrG*mC*mG*mA |
| MG119-129 sgRNA | 1011 | MG119-129 | Nucleotide | Artificial | | mU*mA*mC*rArCrArUrCrArUrUrArGrArUrGrUrGrArGrGrGrCrArArUrUrArUrArU |
| targeting APOA1 | | hAPOA1 | | sequence | | GrUrCrCrUrArArUrGrGrUrGrArGrGrArUrGrCrArGrArUrUrArArArGrCrArUrUrArG |
| | | sgRNA A5 | | | | GrUrUrCrArUrGrArArCrCrArArCrArArUrGrUrArCrUrGrUrArArUrCrArCrUrCrGrCI |
| | | | | | | UrArArArUrCrGrUrGrUrArArGrArArArUrUrArCrArUrGrGrUrArCrUrArArArArCrA |
| | | | | | | UrGrUrGrGrArCrGrCrGrCrUrGrCrGrCrA*mC*mG*mC |
| MG119-129 sgRNA | 1012 | MG119-129 | Nucleotide | Artificial | | mU*mA*mC*rArCrArUrCrArUrUrArGrArUrGrUrGrArGrGrGrCrArArUrUrArUrArU |
| targeting APOA1 | | hAPOA1 | | sequence | | GrUrCrCrUrArArUrGrGrUrGrArGrGrArUrGrCrArGrArUrUrArArArGrCrArUrUrArG |
| | | sgRNA B5 | | | | GrUrUrCrArUrGrArArCrCrArArCrArArUrGrUrArCrUrGrUrArArUrCrArCrUrCrGrC |
| | | | | | | UrArArArUrCrGrUrGrUrArArGrArArArUrUrArCrArUrGrGrUrArCrUrArArArArCrAr |
| | | | | | | GrUrGrGrArCrGrCrGrCrUrGrCrGrCrArC*mG*mC*mA |
| MG119-129 sgRNA | 1013 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | ArCrArGrCrGrArCrGrArGrCrUrGrCrGrC*mC*mA*mG |
| MG119-129 sgRNA | 1014 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA D5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | CrArGrCrGrArCrGrArGrCrUrGrCrGrCrC*mA*mG*mC |
| MG119-129 sgRNA | 1015 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA E5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrArGrGrCrUrCrUrCrArArGrGrArGrArA*mC*mG*mG |
| MG119-129 sgRNA | 1016 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA F5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrUrArCrCrArCrGrCrCrArArGrGrCrCrA*mC*mC*mG |
| MG119-129 sgRNA | 1017 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA G5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrA |
| | | | | | | GrGrCrCrArCrCrGrArGrCrArUrCrUrGrA*mG*mC*mA |
| MG119-129 sgRNA | 1018 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA H5 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrArUrCrUrGrArGrCrArCrGrCrUrCrA*mG*mC*mG |
| MG119-129 sgRNA | 1019 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA A6 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrCrCrGrCrGrCrUrCrGrArGrGrArCrC*mU*mC*mC |
| MG119-129 sgRNA | 1020 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA B6 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrGrCrCrUrGrCrUrGrCrCrCrGrUrGrCrU*mG*mG*mA |
| MG119-129 sgRNA | 1021 | MG119-129 | Nucleotide | Artificial | | mU*mA*mC*rArCrArUrCrArUrUrArGrArUrGrUrGrArGrGrGrCrArArUrUrArUrArUr |
| targeting APOA1 | | hAPOA1 | | sequence | | GrUrCrCrUrArArUrGrGrUrGrArGrGrArUrGrCrArGrArUrUrArArArGrCrArUrUrArGr |
| | | sgRNA C6 | | | | GrUrUrCrArUrGrArArCrCrArArCrArArUrGrUrArCrUrGrUrArArUrCrArCrUrCrGrCr |
| | | | | | | UrArArArUrCrGrUrGrUrArArGrArArArUrUrArCrArUrGrGrUrArCrUrArArArArCrAr |
| | | | | | | GrCrUrGrGrArGrArGrCrUrUrCrArArGrG*mU*mC*mA |
| DNA Sequence of | 1022 | MG119-129 | Nucleotide | Artificial | | GAAGCTCTCCAGCACGGGCA |
| APOA1 Target Site | | hAPOA1 | | sequence | | |
| | | Target Site | | | | |
| | | A1 | | | | |
| DNA Sequence of | 1023 | MG119-129 | Nucleotide | Artificial | | GGCGGAGGTCCTCGAGCGCG |
| APOA1 Target Site | | hAPOAl | | sequence | | |
| | | Target Site | | | | |
| | | B1 | | | | |
| DNA Sequence of | 1024 | MG119-129 | Nucleotide | Artificial | | CTCGCTGAGCGTGCTCAGAT |
| APOA1 Target Site | | hAPOA1 | | sequence | | |
| | | Target Site | | | | |
| | | C1 | | | | |
| DNA Sequence of | 1025 | MG119-129 | Nucleotide | Artificial | | GGCGTGGTACTCGGCCAGTC |
| APOA1 Target Site | | hAPOA1 | | sequence | | |
| | | Target Site | | | | |
| | | D1 | | | | |
| DNA Sequence of | 1026 | MG119-129 | Nucleotide | Artificial | | TCTCCTTGAGAGCCTCAAGG |
| APOA1 Target Site | | hAPOA1 | | sequence | | |
| | | Target Site | | | | |
| | | E1 | | | | |
| DNA Sequence of | 1027 | MG119-129 | Nucleotide | Artificial | | GAGAGCCTCAAGGCGCGCGG |
| APOA1 Target Site | | hAPOA1 | | sequence | | |
| | | Target Site | | | | |
| | | F1 | | | | |
| DNA Sequence of | 1028 | MG119-129 | Nucleotide | Artificial | | GCGCTGGCGCAGCTCGTCGC |
| APOA1 Target Site | | hAPOA1 | | sequence | | |
| | | Target Site | | | | |
| | | G1 | | | | |
| DNA Sequence of | 1029 | MG119-129 | Nucleotide | Artificial | | GTGGGCTCAGCTTCTCTTGC |
| APOA1 Target Site | | hAPOA1 | | sequence | | |
| | | Target Site | | | | |
| | | H1 | | | | |
| DNA Sequence of | 1030 | MG119-129 | Nucleotide | Artificial | | CTTGGAGCTCTGCGCGCAGC |
| APOA1 Target Site | | hAPOA1 | | sequence | | |
| | | Target Site | | | | |
| | | A2 | | | | |
| DNA Sequence of | 1031 | MG119-129 | Nucleotide | Artificial | | CTGGCGGTAGAGCTCCATCT |
| APOA1 Target Site | | hAPOA1 | | sequence | | |
| | | Target Site | | | | |
| | | B2 | | | | |
| DNA Sequence of | 1032 | MG119-129 | Nucleotide | Artificial | | CTCCTCCTGCCACTTCTTCT |
| APOA1 Target Site | | hAPOA1 | | sequence | | |
| | | Target Site | | | | |
| | | C2 | | | | |
| DNA Sequence of | 1033 | MG119-129 | Nucleotide | Artificial | | GGCCTTCACCTCCTCCAGAT |
| APOA1 Target Site | | hAPOA1 | | sequence | | |
| | | Target Site | | | | |
| | | D2 | | | | |
| DNA Sequence of | 1034 | MG119-129 | Nucleotide | Artificial | | CACCTCCTCCAGATCCTTGC |
| APOA1 Target Site | | hAPOA1 | | sequence | | |
| | | Target Site | | | | |
| | | E2 | | | | |
| DNA Sequence of | 1035 | MG119-129 | Nucleotide | Artificial | | GCTCATCTCCTGCCTCAGGC |
| APOA1 Target Site | | hAPOA1 | | sequence | | |
| | | Target Site | | | | |
| | | F2 | | | | |
| DNA Sequence of | 1036 | MG119-129 | Nucleotide | Artificial | | CTGTCTCCTTTTCCAGGTTA |
| APOA1 Target Site | | hAPOA1 | | sequence | | |
| | | Target Site | | | | |
| | | G2 | | | | |
| DNA Sequence of | 1037 | MG119-129 | Nucleotide | Artificial | | TTCCAGGTTATCCCAGAACT |
| APOA1 Target Site | | hAPOA1 | | sequence | | |
| | | Target Site | | | | |
| | | H2 | | | | |
| DNA Sequence of | 1038 | MG119-129 | Nucleotide | Artificial | | GAACTCCTGGGTCACAGGGC |
| APOA1 Target Site | | hAPOA1 | | sequence | | |
| | | Target Site | | | | |
| | | A3 | | | | |
| DNA Sequence of | 1039 | MG119-129 | Nucleotide | Artificial | | GCTGTTCGCGCAGCTTGCTG |
| APOA1 Target Site | | hAPOA1 | | sequence | | |
| | | Target Site | | | | |
| | | B3 | | | | |
| DNA Sequence of | 1040 | MG119-129 | Nucleotide | Artificial | | GTTGTCAAGGAGCTTTAGGC |
| APOA1 Target Site | | hAPOA1 | | sequence | | |
| | | Target Site | | | | |
| | | C3 | | | | |
| DNA Sequence of | 1041 | MG119-129 | Nucleotide | Artificial | | TCTGTCTCACCCTCCAGCCT |
| APOA1 Target Site | | hAPOA1 | | sequence | | |
| | | Target Site | | | | |
| | | D3 | | | | |
| DNA Sequence of | 1042 | MG119-129 | Nucleotide | Artificial | | CTGTCTCACCCTCCAGCCTA |
| APOA1 Target Site | | hAPOA1 | | sequence | | |
| | | Target Site | | | | |
| | | E3 | | | | |
| DNA Sequence of | 1043 | MG119-129 | Nucleotide | Artificial | | CCAGCCTAAAGCTCCTTGAC |
| APOA1 Target Site | | hAPOA1 | | sequence | | |
| | | Target Site | | | | |
| | | F3 | | | | |
| DNA Sequence of | 1044 | MG119-129 | Nucleotide | Artificial | | AAGCTCCTTGACAACTGGGA |
| APOA1 Target Site | | hAPOA1 | | sequence | | |
| | | Target Site | | | | |
| | | G3 | | | | |
| DNA Sequence of | 1045 | MG119-129 | Nucleotide | Artificial | | GACAACTGGGACAGCGTGAC |
| APOA1 Target Site | | hAPOA1 | | sequence | | |
| | | Target Site | | | | |
| | | H3 | | | | |
| DNA Sequence of | 1046 | MG119-129 | Nucleotide | Artificial | | CAGCAAGCTGCGCGAACAGC |
| APOA1 Target Site | | hAPOA1 | | sequence | | |
| | | Target Site | | | | |
| | | A4 | | | | |
| DNA Sequence of | 1047 | MG119-129 | Nucleotide | Artificial | | GTGACCCAGGAGTTCTGGGA |
| APOA1 Target Site | | hAPOA1 | | sequence | | |
| | | Target Site | | | | |
| | | B4 | | | | |
| DNA Sequence of | 1048 | MG119-129 | Nucleotide | Artificial | | GGAGTTCTGGGATAACCTGG |
| APOA1 Target Site | | hAPOA1 | | sequence | | |
| | | Target Site | | | | |
| | | C4 | | | | |
| DNA Sequence of | 1049 | MG119-129 | Nucleotide | Artificial | | GGTGCAGCCCTACCTGGACG |
| APOA1 Target Site | | hAPOA1 | | sequence | | |
| | | Target Site | | | | |
| | | D4 | | | | |
| DNA Sequence of | 1050 | MG119-129 | Nucleotide | Artificial | | ACCTGGACGACTTCCAGAAG |
| APOA1 Target Site | | hAPOA1 | | sequence | | |
| | | Target Site | | | | |
| | | E4 | | | | |
| DNA Sequence of | 1051 | MG119-129 | Nucleotide | Artificial | | CCTGGACGACTTCCAGAAGA |
| APOA1 Target Site | | hAPOA1 | | sequence | | |
| | | Target Site | | | | |
| | | F4 | | | | |
| DNA Sequence of | 1052 | MG119-129 | Nucleotide | Artificial | | GAGGGCGCGCGCCAGAAGCT |
| APOA1 Target Site | | hAPOA1 | | sequence | | |
| | | Target Site | | | | |
| | | G4 | | | | |
| DNA Sequence of | 1053 | MG119-129 | Nucleotide | Artificial | | CTGGGCGAGGAGATGCGCGA |
| APOA1 Target Site | | hAPOA1 | | sequence | | |
| | | Target Site | | | | |
| | | H4 | | | | |
| DNA Sequence of | 1054 | MG119-129 | Nucleotide | Artificial | | TGTGGACGCGCTGCGCACGC |
| APOA1 Target Site | | hAPOA1 | | sequence | | |
| | | Target Site | | | | |
| | | A5 | | | | |
| DNA Sequence of | 1055 | MG119-129 | Nucleotide | Artificial | | GTGGACGCGCTGCGCACGCA |
| APOA1 Target Site | | hAPOA1 | | sequence | | |
| | | Target Site | | | | |
| | | B5 | | | | |
| DNA Sequence of | 1056 | MG119-129 | Nucleotide | Artificial | | ACAGCGACGAGCTGCGCCAG |
| APOA1 Target Site | | hAPOA1 | | sequence | | |
| | | Target Site | | | | |
| | | C5 | | | | |
| DNA Sequence of | 1057 | MG119-129 | Nucleotide | Artificial | | CAGCGACGAGCTGCGCCAGC |
| APOA1 Target Site | | hAPOA1 | | sequence | | |
| | | Target Site | | | | |
| | | D5 | | | | |
| DNA Sequence of | 1058 | MG119-129 | Nucleotide | Artificial | | GAGGCTCTCAAGGAGAACGG |
| APOA1 Target Site | | hAPOAl | | sequence | | |
| | | Target Site | | | | |
| | | E5 | | | | |
| DNA Sequence of | 1059 | MG119-129 | Nucleotide | Artificial | | GTACCACGCCAAGGCCACCG |
| APOA1 Target Site | | hAPOA1 | | sequence | | |
| | | Target Site | | | | |
| | | F5 | | | | |
| DNA Sequence of | 1060 | MG119-129 | Nucleotide | Artificial | | GGCCACCGAGCATCTGAGCA |
| APOA1 Target Site | | hAPOA1 | | sequence | | |
| | | Target Site | | | | |
| | | G5 | | | | |
| DNA Sequence of | 1061 | MG119-129 | Nucleotide | Artificial | | GCATCTGAGCACGCTCAGCG |
| APOA1 Target Site | | hAPOA1 | | sequence | | |
| | | Target Site | | | | |
| | | H5 | | | | |
| DNA Sequence of | 1062 | MG119-129 | Nucleotide | Artificial | | GCCCGCGCTCGAGGACCTCC |
| APOA1 Target Site | | hAPOAl | | sequence | | |
| | | Target Site | | | | |
| | | A6 | | | | |
| DNA Sequence of | 1063 | MG119-129 | Nucleotide | Artificial | | GGCCTGCTGCCCGTGCTGGA |
| APOA1 Target Site | | hAPOA1 | | sequence | | |
| | | Target Site | | | | |
| | | B6 | | | | |
| DNA Sequence of | 1064 | MG119-129 | Nucleotide | Artificial | | GCTGGAGAGCTTCAAGGTCA |
| APOA1 Target Site | | hAPOA1 | | sequence | | |
| | | Target Site | | | | |
| | | C6 | | | | |
| MG191 effectors | 1065 | MG191-1 | pR0tein | Unknown | uncultivated | MIKSIKLKVKGDCPITKDVINEYKEYYNRCSDWIKNNLTSITIGEIGKFLQDVTGK |
| | | effector | | | organism | TOGYIKVALSDEWKDKPMYNLFTKDYNAEHANNLLYYFIRKNNFDRYDGNILN |
| | | | | | | ISDTYYRQNGYFKLVAGNYRTKIRTLNCKIKRKKVDTDSTSEDIESQVMYEILNR |
| | | | | | | SLNKKSDWDSFISYIENVENPNIDSINRYTLLRDYFCDNEDVIKNKIELLSIEQLKD |
| | | | | | | FGGCIMKQHINTMTLIIQHFKIEEKENSLGLIFNLPLSKKQYQIELWGNRQINKGT |
| | | | | | | KERDAFLNTYGENIVFTINNDELYVVFSYESEFGKEETNFEKSVGLDINFKHALF |
| | | | | | | VTSEMDNDQFDGYINLYKYILSHSEFINLLTEDERKDYEELSKVVTFCPFENQLLF |
| | | | | | | ARYDKMCKFCKKEQVLSKLLYALQKKLKNENRTKEYIYVSCVNKLRAKYISYF [symbol] |
| | | | | | | LKEKYYEKQKEYDIEMGFVDDSTESKESMDRRRFENPFRNTFVANELLAKMSK |
| | | | | | | VQQDINGCLSNIINYVYKVFEQNGYNIIALENLENSNFEKRQVLPTIKSLLKYHKL |
| | | | | | | ENQNINDIKASDKIKEYIENGYYSFITNENNEIVDAKYTAKGDIKVKNAKFFNLM |
| | | | | | | MKILHFASIKDEFVLLSNNGKSQIALVPSEYTSQMDSIDHCIYMTENDKGKIVKV |
| | | | | | | DKRKVRTKQERHINGLNADFNAANNIKYIVSNEKWRKVFCTPKKAKYNTPALD |
| | | | | | | ATKKGQFRILDDMKKLNATKLLEIEK* |
| MG191 effectors | 1066 | MG191-2 | pR0tein | Unknown | uncultivated | MSKQTKTIELKVKGDCPITKDVINEYNECYNRCSNWINNNLTNITIGEIAKFLKDT |
| | | effector | | | organism | TGKTAGYIEVALSDKWINKPMYYLFTDQYNENHANNLLYSFIKKNNFDMYDGN |
| | | | | | | ILNISDTNYRKYGYFKLVASNYRTKIRTLNCKIKRKKVDVDSTSEDIENQVMYEV |
| | | | | | | INNSLNKKTDWDNYISYLENVENPNIDIINRYKLLCDYFCENEDVIKNKIELLSIE |
| | | | | | | QLKDFGGCIRKQHINTITVIIQRFKIEEKENSFGFILHLPLNKKSYPIELWGHRKIN |
| | | | | | | KGTKERDAFINTYGENIVFRINADESYVSI.SYESEFEKEETNFEKIVGLDINFKHA |
| | | | | | | LFVSSEQDSEQLNGYINLYKYILSHNEFTNLLTEDERKDYEELSKIVTFCPFEYQL |
| | | | | | | LFARYDKMSKYYVKEQVFSKILYSLQKKLKYEKRTKEYIYVSCVNKLRAKYKS |
| | | | | | | YFILKEKYHEKQSEYDIEMGFVDDSTESKESMDKRRFENPFRNTLIANELLAKM |
| | | | | | | NNVQQDINGCLKNIINYVYKVFEQNGYDTTGLENLENSNFEKRTELPRIKSLLGY |
| | | | | | | HKLKNQNINDIKASDKIKEYIENGYYELITNENNEIIDAKYTTKGDIKVKSADFFM |
| | | | | | | LMMKTLHFASIKDEFVLLSNNGKNQIALVPSEYTSQMDSIDHCIYMTENNKGKT |
| | | | | | | VKVDKRKVRTKQERHINGLNADFNAANNIKYMVSNEKWRNVFCTQKKAKYN [symbol] |
| | | | | | | PTLDATKKGQFRILEELKKLKATKLLEMEK* |
| MG191 effectors | 1067 | MG191-3 | pR0tein | unknown | uncultivated | MAKSIMKKSIKFKVKGNSPINEDVINEYKGYYNTCSNWINNNLTSITIGEMGKFI |
| | | effector | | | organism | KDVMCKTTVYIDVALSDEWKDKPMYYLFTKKYNPKHANNLLYYFIKEKKLDK |
| | | | | | | FNGNILNIPESYYRKEGYFKLVAGNYRTKINKLNCKIKSKKVDANSLSEDIEMQT |
| | | | | | | IYEIVKRGFDKKSDWDSYISYIECVQNPNIDNINRYKLLRDYFCENEDVIKNKIEII |
| | | | | | | SIEQIKQFGGCIMKPHINSMTFGIKKFKIEEIENSLGFTFNLPLNKNNYKIELWGHR |
| | | | | | | QLKKGNKESNVNVSLFDFINTYGQNVVFTIKRKKLYVVFSYDYEFEKGECNFEK |
| | | | | | | SVGLDVNFKHSLFVTSEIDNNQFDGYINLYKYILSNNEFTSLLTDSERKDYEDLA |
| | | | | | | NIVTFCPFEYQLLFSRYDKMSKFSEKEKVLSKILYSLQKKLKNENRTKEYIYVSC |
| | | | | | | VNKLRAKYVSYFKLIQKYNEKQKEYDIEMGFVDDSTESKESMDKRRFENPFTNT |
| | | | | | | PVANELLEKMNNVKQDINGCKKNIVVYAYKVLEQNGYNIIALENLENSNFEKIR |
| | | | | | | VLPKIKSLLEHHKFENKNINDIKNSDKYKEFIEPGYFELITNENNEIIDAKYTQKG |
| | | | | | | DIKIKNADFINRMIKALNFASIKDEFILLSHNGKSQIALVPAEYTSQMDSIDHCIYM |
| | | | | | | TKNDKGKLVKVDKRKVRTKQERIIINGLNADFNAACNIKYIVTNEDWRKVFCIK |
| | | | | | | PKKEDYNTPLLDATKNGQFRILDKLKKLNATKLLEMEK* |
| MG191 effectors | 1068 | MG191-4 | pR0tein | Unknown | uncultivated | MAKHSKVQTGYITKTFTFKVKDSKNIDKDTLIDAFKEYQNYYNECSDTICAHLT |
| | | effector | | | organism | DMTIGDLFAYIPEESQNTGYAQWASSDEWKNKPLYCIFGKQFLGSHRDNLLYLF |
| | | | | | | LINRVKYTGNKLNIPATFYRRQGFIKCVISNYTSVFTRIKPRITFHKLTGDETHENL |
| | | | | | | VLQTICDMIKFNLEDEQKWAEQVQYFEMKNETSKESLDRIHTLFNFYKDNALEI |
| | | | | | | ANKYNELITESLCKFGGCHRDDSKLTMSIQLSKKSIKVTHSGDNTLNYKFSKTFN |
| | | | | | | LDLWGRKDVITNNELLIDLNNVCEAIIFKVKKDEIYVDIPFKVAFNKKESTLDKIV |
| | | | | | | GVDANIKHMLLSTSLKDNGGYIGYTNLYKEVVNDPDFQKVCDKTTMKILKDISK |
| | | | | | | TVTFAPIEFDMLFSRISKQRKIEDKYIDMEIVFTNVLNKLKQKFIVSNDNKNRIYIE |
| | | | | | | SILKMRSQLKSFAILEEVKYQKASEYDSAIIEKFGVEYLEAHPFKNTETYKEISKK |
| | | | | | | TLNIAEKIIGCRNNIIQYAYKIFEANGFDMISLEKLTNSNFKKEKNMPSIKSLLFYH |
| | | | | | | HVLGKTNEEIEKLDAYSVIKKGYYVFEYEDDKVVNAKYSVTGEMNKIKTTMFN |
| | | | | | | LMIKSIHFAEIKDYFITLANNGGVGVSLVPSYYTSQMDSIDHKVFGVLSKKGKWT |
| | | | | | | LADKRKVRKNQETHINGLNADHNAANNIAFILFDEKWRNKFTKQTKTPKYNTA |
| | | | | | | SYDTSINSQGKMLQALKSLKAFKEFEC* |
| MG191 effectors | 1069 | MG191-5 | pR0tein | unknown | uncultivated | MSKQTKTIVLKVKGDCPITKDVINEYNECYNRCSNWINNNLTNITIGEIAKFLKD |
| | | effector | | | organism | TTGKTAGYIEVALSDKWINKPMYYLFTDQYNENHANNLLYSFIKKNNFDMYDC |
| | | | | | | NILNISDTNYRKYGYFKLVASNYRTKIRTLNCKIKRKKVDVDSTSEDIENQVMY [symbol] |
| | | | | | | VINNSLNKKTDWDNYISYLENVENPNIDIINRYKLLCDYFCENEDVIKNKIELLSI |
| | | | | | | EQLKDFGGCIRKQHINTITVIIQRFKIEEKENSFGFILHLPLNKKSYPIELWGHRKI [symbol] |
| | | | | | | KGTKERDAFLNTYGENIVFRINADESYVSLSYESEFEKEETNFEKIVGLDINFK [symbol] |
| | | | | | | LFVSSEQDSEQLNGYINLYKYILSHNEFTNLLTEDERKDYEELSKIVTFCPFEYQL |
| | | | | | | LFARYDKMSKYYVKEQVFSKILYSLQKKLKYEKRTKEYIYVSCVNKLRAKYKS |
| | | | | | | YFILKEKYHEKQSEYDIEMGFVDDSTESKESMDKRRFENPFRNTLIANELLAKM |
| | | | | | | NNVQQDINGCLKNIINYVYKVFEQNGYDTTGLENLENSNFEKRTELPRIKSLLGY |
| | | | | | | HKLKNQNINDIKASDKIKEYIENGYYELITNENNEIIDAKYTTKGDIKVKSADFF [symbol] |
| | | | | | | LMMKTLHFASIKDEFVLLSNNGKNQIALVPSEYTSQMDSIDHCIYMTENNKGK [symbol] |
| | | | | | | VKVDKRKVRTKQERHINGLNADFNAANNIKYMVSNEKWRNVFCTQKKAKYNT |
| | | | | | | PTLDATKKGQFRILEELKKLKATKLLEMEK* |
| MG191 effectors | 1070 | MG191-6 | pR0tein | Unknown | uncultivated | MAHKKNVGAEIVKTYSFKVKNTNGITMEKLMNAIDEFQSYYNLCSDWICKNLT |
| | | effector | | | organism | TMTIGDLDQYIPEKAKGNTYATVLLDEAWKNQPLYKIFSKKYSSNNRNNALYC |
| | | | | | | ALSSVIDTNKENVLGFSKTYYTRNGYILNVISNYASKLSKLNTGVKSRAIKETSD |
| | | | | | | EATIIEQIIYEMEHNKWESIEDWKNQIEYLNSKTDYNPTYMERMKTLSAYYSAH |
| | | | | | | KSEVDAKMQEMAVENLVKFGGCRRNNSKKSMFIMGASNTNYTISYIGDNSFNIN |
| | | | | | | FANILNFDVYGRRDVVKNGEVLVDIMANHRDSIVLKIVNGELYADVPCSVTLNK |
| | | | | | | VESNFEKVVGIDVNMKHMLLSTSVTDNGSSDFLNIYKEMSNNAEFMALCPEEDR |
| | | | | | | KYYKDISQYVTFAPLELDLLFSRISKQGKVKMEKAYSEILEALKWKFFANGDNK |
| | | | | | | NRIYVESIQKIRQQIKALCVIKNAYYEQQSAYDIDKTQEYIETHPFSLTEKGMSIKS |
| | | | | | | KMDKICQTIIGCRNNIIDYAYSFFEKNGYSIIGLEKLTSSQFEKTKSMPTCKSLLNF |
| | | | | | | HKVLGHTLSELETLPINDVVKKGYYTFTTDNEGKITDASLSEKGKVRKMKDDFF |
| | | | | | | NQAIKAIHFADVKDYFATLSNNGQTGIFFVPSQFTSQMDSNTHNLYFENAKNGG |
| | | | | | | LKLAPKYKVRQTQEYHLNGLPADYNAARNIAYIGLDETMRNTFLKKANSNKSL |
| | | | | | | YNQPIYDTGIKKTAGVFSRMKKLKRYEII* |
| MG191 effectors | 1071 | MG191-7 | pR0tein | Unknown | uncultivated | MASIIKKTESNQIIKTFSFKIKNANGLSLDVLNDAITEYQNYYNICSDWIKDHLTM |
| | | effector | | | organism | KISELYKYIPNEKKNSGYALTLISDEWKDKPMYMMFKKGYPANNRDNAIYETL |
| | | | | | | NTCNTEHYTGNILNFSDTYYRRFGYVASAISNYVTKISKMSTGSRSKNISNDSDV |
| | | | | | | DTIMEQVIYEMEHNGWTSVKDWENQMEYLESKTDSNPNFVYRMTTLYEFYKS |
| | | | | | | HIDDVNSKMETMSIDSLIKFGGCRRKDSKKSMYIMGGSNTPFDITQIGGNSLNIKF |
| | | | | | | SKNLNVDVFGRYDVIKDNTLLVDIINGHGASFVLKIINDEIYIDINVSVPFDKKIAT |
| | | | | | | TNKVVGIDVNIKHMLLATNILDDGNVKGYVNIYKEVINDSDFKKVCNSTVMQY |
| | | | | | | FTDFSKFVTFCPLEFDFLFSRVCNQKGIYNDNSAMEKSFSDVLNKlKWNFIETGD |
| | | | | | | NTKRIYIENVMKLRSQMKAYAIVKNAYYKQQSEYDFGKSEEFIQEHPFSNTDKG [symbol] |
| | | | | | | EILHKLDNISKKILGCRNNIIQYSYNLFEINGYDMISLEKLTSSQFKKKPFPTVNSL |
| | | | | | | LKYHKILGCTQEEMEKKDIYSVIKKGYYDIIFDNDVVTDAKLSTKGELSKFKDD [symbol] |
| | | | | | | FNLMIKSIHFADIKDYFITLSNNGTVGVSLVPSYFTSQMDSIDHKIYFVQDNKS [symbol] |
| | | | | | | LKLANKHKVRSSQEKHINGLNADYNAARNIAYIMENNECRNMFMKQSRTDKSI |
| | | | | | | YNKPSYETFIKTQGSAVSKLKKDGFVKILDEASV* |
| MG191 effectors | 1072 | MG191-8 | pR0tein | unknown | uncultivated | MASHKKTESNQIIKTFSFKLKNANGLSLDVLNDAITEYQNYYNICSDWIKDHLT |
| | | effector | | | organism | MKIGELYKYIPDEKKNSGYALTLISDEWKDKPMYMMFKKGYPANNRDNAIYET |
| | | | | | | LNTCNTEHYTGNILNFSDTYYRRFGYVASAISNYVTKISKMSTGSRSKNISNDSD |
| | | | | | | VDTIMEQVIYEMEHNGWTSVKDWENQMEYLESKTDSNPNFVYRMTTLYEFYK |
| | | | | | | SHIDEVNSKMETMSIDSLIKFGGCRRKDSKKSMYIMGGSNTPFDITQIGGNSLNI [symbol] |
| | | | | | | FSKNLNVDVFGRYDVIKDNTLLVDIINGHGASFVLKIINDEIYIDINVSVPFDKKIA |
| | | | | | | TTNKVVGIDVNIKHMLLATNILDDGNVKGYVNIYKEVINDSDFKKVCNSTIMQY |
| | | | | | | FTDFSKFVTFCPLEFDFLFSRVCNQKGIYNDNSAMEKSFSDVLNKLKWNFIETGD |
| | | | | | | NTKRIYIENVMKLRSQMKAYAIVKNAYYKQQSEYDFGKSEEFIQEHPFSNTDKGI |
| | | | | | | EILHKLDNISKKILGCRNNIIQYSYNLFEINGYDMISLEKLTSSQFKKKPFPTVNSL |
| | | | | | | LKYHKILGCTQEEMEKKDIYSVIKKGYYDIIFDNGVVIDAKLSAKGELSKFKDDF |
| | | | | | | FNLMIKSIHFADIKDYFITLSNNGTAGVSLVPSFFTSQMDSIDHKIYFVQDNKSGK |
| | | | | | | LKLANKHKVRSSQEKHINGLNADYNAARNIAYIMENTDCRNMFMKQSRTDKSL |
| | | | | | | YNKPSYETFIKTQGSAVSKLKKDGFVKILDEASV* |
| MG191 effectors | 1073 | MG191-9 | pR0tein | Unknown | uncultivated | MASHKKTESNQIIKTFSFKIKNANGLSLDVLNDAITEYQNYYNICSDWIKDHLTM |
| | | effector | | | organism | KIGELYKYIPDEKKNSGYALTLISDEWKDKPMYMMFKKGYPANNRDNAIYETL |
| | | | | | | NTCNTEHYTGNILNFSDTYYRRFGYVASAISNYVTKISKMSTGSRSKNISNDSDV |
| | | | | | | DTIMEQVIYEMEHNGWTSVKDWENQMEYLESKTDSNPNFVYRMTTLYEFYKS |
| | | | | | | HIDEVNSKMETMSIDSLIKFGGCRRKDSKKSMYIMGGSNTPFDITQIGGNSLNIKF |
| | | | | | | SKNLNVDVFGRYDVIKDNTLLVDIINGHGASFVLKIINDEIYIDINVSVPFDKKIAT |
| | | | | | | TNKVVGIDVNIKHMLLATNILDDGNVKGYVNIYKEVINDSDFKKVCNSTVMQY |
| | | | | | | FTDFSKFVTFCPLEFDFLFSRVCNQKGIYNDNSAMEKSFSDVLNKLKWNFIETGD |
| | | | | | | NTKRIYIENVMKLRSQMKAYAIVKNAYYKQQSEYDFGKSEEFIQEHPFSNTDKGI |
| | | | | | | EILHKLDNISKKILGCRNNIIQYSYNLFEINGYDMISLEKLTSSQFKKKPFPTVNSL |
| | | | | | | LKYHKILGCTQEEMEKKDIYSVIKKGYYDIIFDNDVVTDAKLSTKGELSKFKDDF |
| | | | | | | FNLMIKSIHFADIKDYFITLSNNGTAGVSLVPSYFTSQMDSIDHKIYFVQDNKSGK |
| | | | | | | LKLANKHKVRSSQEKHINGLNADYNAARNIAYIMENTECRNMFMKQSRTDKSL |
| | | | | | | YNKPSYETFIKTQGSAVSKLKKDGFVKILDEASV* |
| MG191 effectors | 1074 | MG191-10 | pR0tein | unknown | uncultivated | MRISPHLFFIFFKKIWKCHIFVLSLYQLNQYIMASHKKTESNQIIKTFSFKIKNANG |
| | | effector | | | organism | LSLDVLNDAITEYQNYYNICSDWIKDHL.TMKIGELYKYIPDEKKNSGYALTLISD |
| | | | | | | EWKDKPMYMMFKKGYPANNRDNAIYETLNTCNTEHYTGNILNFSDTYYRRFG |
| | | | | | | YVASAISNYVTKISKMSTGSRSKNISNDSDVDTIMEQVIYEMEHNGWTSVKDWF |
| | | | | | | NQMEYLESKTDSNPNFVYRMTTLYEFYKSHIDEVNSKMETMSIDSLIKFGGCRR |
| | | | | | | KDSKKSMYIMGGSNTPFDITQIGGNSLNIKFSKNLNVDVFGRYDVIKDNTLLVD [symbol] |
| | | | | | | NGHGASFVLKIINDEIYIDINVSVPFDKKIATTNKVVGIDVNIKHMLLATNILDDG |
| | | | | | | NVKGYVNIYKEVINDSDFKKVCNSTVMQYFTDFSKFVTFCPLEFDFLFSRVCSQ [symbol] |
| | | | | | | GIYNDNSAMEKSFSDVLNKLKWNFIETGDNTKRIYIENVMKLRSQMKAYAIVK [symbol] |
| | | | | | | AYYKQQSEYDFGKSEEFIQEHPFSNTDKGIEILHKLDNISKKILGCRNNIIQYSYN [symbol] |
| | | | | | | FEINGYDMISLEKLTSSQFKKKPFPTVNSLLKYHKILGCTQEEMEKKDIYSVIKK [symbol] |
| | | | | | | YYDIIFDDDVVTDAKLSTKGELSKFKDDFFNLMIKSIHFADIKDYFITLSNNGTA [symbol] |
| | | | | | | VSLVPSYFTSQMDSIDHKIYFVQDNKSGKLKLANKHKVRSSQEKHINGLNADY [symbol] |
| | | | | | | AARNIAYIMENNECRNMFMKQSRTDKSLYNKPSYETFIKTQGSAVSKLKKDGFV |
| | | | | | | KILDEASV* |
| MG191 effectors | 1075 | MG191-11 | pR0tein | Unknown | uncultivated | MASHKKTESNQIIKTFSFKIKNANGLSLDVLNDAITEYQNYYNICSDWIKDHLTM |
| | | effector | | | organism | KIGELYKYIPNEKKNSGYALTLISDEWKDKPMYMMFKKGYPANNRDNAIYETL |
| | | | | | | NTCNTEHYTGNILNFSDTYYRRFGYVASAISNYVTKISKMSTGSRSKNISNDSDV |
| | | | | | | DTIMEQVIYEMEHNGWTSVKDWENQMEYLESKTDSNPNFVYRMTTLYEFYKS |
| | | | | | | HIDEVNSKMETMSIDSLIKFGGCRRKDSKKSMYIMGGSNTPFDITQIGGNSLNIKF |
| | | | | | | SKNLNVDVFGRYDVIKDNTLLVDIINGHGASFVLKIINDEIYIDINVSVPFDKKIAT |
| | | | | | | TNKVVGIDVNIKHMLLATNILDDGNVNGYVNIYKEVINDSDFKKVCNSTVMKY |
| | | | | | | FTDFSKFVTFCPLEFDFLFSRVCNQKGIYNDNSAMEKSFSDVLNKLKWNFIETGD |
| | | | | | | NTKRIYIENVMKLRSQMKAYAIVKNAYYKQQSEYDFGKSEEFIQEHPFSNTDKGI |
| | | | | | | EILHKLDNISKKILGCRNNIIQYSYNLFEINGYDMISLEKLTSSQFKKKPFPTVNSL |
| | | | | | | LKYHKILGCTQEEMEKKDIYSVIKKGYYDIIFDNDVVTDAKLSAKGELSKFKDDF |
| | | | | | | FNLMIKSIHFADIKDYFITLSNNGTVGVSLVPSYFTSQMDSIDHKIYFVQDNKSGK |
| | | | | | | LKLANKHKVRSSQEKHINGLNADYNAARNIAYIMENTDCRNMFMKQSRTDKSL |
| | | | | | | YNKPSYETFIKTQGSAVAKLKKEGFVKILDEASV* |
| MG191 effectors | 1076 | MG191-12 | pR0tein | Unknown | uncultivated | MASIIKKTESNQIIKTFSFKIKNANGLSLDVLNDAITEYQNYYNICSDWIKDHLTM |
| | | effector | | | organism | KISELYKYIPNEKKNSGYALTLISDEWKDKPMYMMFKKGYPANNRDNAIYETL |
| | | | | | | NTCNTEHYTGNILNFSDTYYRRFGYVASAISNYVTKISKMSTGSRSKNISNDSDV |
| | | | | | | DTIMEQVIYEMEHNGWTSVKDWENQMEYLESKTDSNPNFVYRMTTLYEFYKS |
| | | | | | | HIDEVNSKMETMSIDSLIKFGGCRRKDSKKSMYIMGGSNTPFDITQIDGNSLNIKF |
| | | | | | | SKNLNVDVFGRYDVIKDNTLLVDIINGHGASFVLKIINDEIYIDINVSVPFDKKIAT |
| | | | | | | TNKVVGIDVNIKHMLLATNILDDGNVKGYVNIYKEIINDSDFKKVCNSTVMQYF |
| | | | | | | TDFSKFVTFCPLEFDFLFSRVCNQKGIYNDNSAMEKSFSDVLNKLKWNFIETGDN |
| | | | | | | TKRIYIENVMKLRSQMKAYAIVKNAYYKQQSEYDFGKSEEFIQEHPFSNTDKGIE |
| | | | | | | ILHKLDNISKKILGCRNNIIQYSYNLFEINGYDMISLEKLTSSQFKKKPFPTVNSLL |
| | | | | | | KYHKILGCTQEEMEKKDIYSVIKKGYYDIIFDNDVVTDAKLSTKGELSKFKDDF [symbol] |
| | | | | | | NLMIKSIHFADIKDYFITLSNNGTAGVSLVPSYFTSQMDSIDHKIYFVQDNKSGK [symbol] |
| | | | | | | KLANKHKVRSSQEKHINGLNADYNAARNIAYIMENNECRNMFMKQSRTDKSL [symbol] |
| | | | | | | NKPSYETFIKTQGSAVAKLKKDGFVKILDEASA* |
| MG191 effectors | 1077 | MG191-13 | pR0tein | unknown | uncultivated | MAHKKNIGAEIVKTYSFKVKNTNGITMEKLMNAIDEYQSYYNLCSDWICKNLT [symbol] |
| | | effector | | | organism | MTIGDLDRYIPEKAKDNIYAIVLLDEAWKNQPLYKIFGKKYSSNNRNNALYCAL [symbol] |
| | | | | | | SSVIDMTKENVLGFSKTHYIRNGYILNVISNYASKLSKLNTGVKSRAIKETSDEA [symbol] |
| | | | | | | IIEQVIYEMEHNKWESIEDWKNQIEYLNSKTDYNPTYMERMKTLSAYYSEHKS [symbol] |
| | | | | | | VDAKMQEMAVENLVKFGGCRRNNSKKSMFIMGSNTTNYTISYIGGNSFNINFA [symbol] |
| | | | | | | ILNFDVYGRRDVVKNGEVLVDIMANHGDSIVLKIVNGELYADVPCSVTLNKVE [symbol] |
| | | | | | | NFDKVVGIDVNMKHMLLSTSVTDNGNSDFVNIYKEMSNNAEFMALCPEKDRK |
| | | | | | | YYKDISQYVTFAPLELDLLFSRISKQGEAKMEKAYSEILESLKWKFFANGDNKN |
| | | | | | | RIYVESIQKIRQQIKALCVIKNAYYEQQSAYDIDKTQEYIETHPFSLTEKGMSIKSK |
| | | | | | | MDKICQTIIGCRNNIIDYAYSFFERNGYSIIGLEKLTSSQFKNTKSMPTCKSLLNLH |
| | | | | | | KVLGHTLSELETLPINDIVKYYTFTTDNEGRITDASLSEKGKIRKMKDRFLNQAIK |
| | | | | | | AIHFADVKDYFATLSNNGQTGIFFVPSQFTSQMDSNTHNLYFEVDKNGGLKMAS |
| | | | | | | KDKTRPKQEYHRNGLPADYNAARNIAYIGLDETMRNTFLKKANSNKSLYNQPIY |
| | | | | | | DTGIKKTAGVFSRMKKLKRYEII* |
| MG191 effectors | 1078 | MG191-14 | pR0tein | Unknown | uncultivated | MASHKKTESNQIIKTFSFKLKNANGLSLDVLNDAITEYQNYYNICSDWIKDHLT |
| | | effector | | | organism | MKIGELYKYIPDEKKNSGYALTLISDEWKDKPMYMMFKKGYPANNRDNAIYET |
| | | | | | | LNTCNTEHYTGNILNFSDTYYRRFGYVASAISNYVTKISKMSTGSRSKNISNDSD |
| | | | | | | VDTIMEQVIYEMEHNGWTSVKDWENQMEYLESKTDSNPNFVYRMTTLYEFYK |
| | | | | | | SHIDDVNSKMETMSIDSLIKFGGYRRKDSKKSMYIMGGSNTPFDITQIGGNSLNIK |
| | | | | | | FSKNLNVDVFGRYDVIKDNTLLVDIINGHGASFVLKIINDEIYIDINVSVPFDKKIA |
| | | | | | | TTNKVVGIDVNIKHMLLATNILDDGNVKGYVNIYKEVINDSDFKKVCNSTVMQ |
| | | | | | | YFTDFSKFVTFCPLEFDFLFSRVCNQKGIYNDNSAMEKSFSDVLNKLKWNFIETG |
| | | | | | | DNTKRIYIENVMKLRSQMKAYAIVKNAYYKHQSEYDFGKSEEFIQEHPFSNTDK |
| | | | | | | GIEILHKLDNISKKILGCRNNIIQYSYNLFEINGYDMISLEKLTSSQFKKKPFPTVNS |
| | | | | | | LLKYHKILGCTQEEMEKKDIYSVIKKGYYDIIFDNDVVTDAKLSTKGELSKFKDD |
| | | | | | | FFNLMIKSIHFADIKDYFITLSNNGTAGVSLVPSFFTSQMDSIDHKIYFVQDNKSG |
| | | | | | | KLKLANKHKVRSSQEKHINGLNADYNAARNIAYIMENNECRNMFMKQSRTDKS |
| | | | | | | LYNKPSYETFIKTQGSAVSKLKKDGFVKILDEASV* |
| MG191 effectors | 1079 | MG191-15 | pR0tein | unknown | uncultivated | MGNKIKQTGTVTKSYKFKSLVGKKEINETWKPAIEEYVSYYNKVSDWICKNLTE |
| | | effector | | | organism | MKIGDLAKYIDSKDTAYYEFVVSDKYKDQPI,YKIFISKATQSVNNALFAAIKKI, |
| | | | | | | NPEGYDGNILGICPSYYRKGGYVVNVISNYRTKMSALSPKIKFKSFDDETVTSEIL |
| | | | | | | MEQCIHDVCKYGIESIDDFKNLLENVSNRENPNLNKVKRLNTLYSFFKGHKDEV |
| | | | | | | LDKMSEYAVEQLKSFNGCRRSGDNVSMTISIQDSKMRKVGNTSFTISVCFNKKN |
| | | | | | | YEIGLLGNRLVVKLDKDGNRVDAVDICENHGEYITFQLKDGVMYVILSSESYFD |
| | | | | | | EKCSEISNVVGVDINIKHMMMATSIVDDGNVKGFLNLYKELVNDRAFMDTCSD |
| | | | | | | AEKEEYIQMSESVNFCPIERDPLFSRASIQMDSMYGTDLVDKKMHNREIAISSVF |
| | | | | | | DRLAKELRVSDPKSASYVDYVKMVRAKYKAYFMLKOKYYSEHSKYDLEQGY [symbol] |
| | | | | | | DESVKSKDTMDKRRFEKNFIDTEIAKELLTKLKNIETDLLGCRDNIINYAFNVFT [symbol] |
| | | | | | | NGYDTISLEYLDSSQFDKYRIVSPSSLLNYHKFEGKKDSDVDKSFINEKNYVFKE [symbol] |
| | | | | | | NENGEIVDIEYSERGLRELKKKDFCNLIIKAIHFADIKDKFVQLANNNKTNIVLV [symbol] |
| | | | | | | SAFSSQMDSKRHKLYVTEVIGKKNKKVTVLVDKRKLRPAQERHINGLNSDFNS [symbol] |
| | | | | | | CNLKYIAENEIFRNIVCDKRKDGEIYGKPFYGIKQCFKKNVSAKMVKVLIENGFT |
| | | | | | | TDFSEANSYATR* |
| MG191 effectors | 1080 | MG191-16 | pR0tein | Unknown | uncultivated | MRCSKRYIDNVSETISNNIEKAAMTSDNHIFRSALKEEYKKIYREKFYPDVTGQP |
| | | effector | | | organism | FPDKGPDNTHIVERTVENLCGQYISALKGVMKASKPKWQDDIFEDESDKVIVEM |
| | | | | | | LMASGNEADIHPKLEKKIASAKKKDKMDEWQKVLDRYDYIKSKTDIEWTYTTL |
| | | | | | | LYDHIARECARSMFEDGNKPKFCYTNGNKFAKQVVDELWGSERVEYEYEKYLH |
| | | | | | | CRTLEAYWNEENKYFFLENQKKSKKTYEARQIIGHFIENEDAVKAETEEYMAKV |
| | | | | | | AKEFGENGGCKFRTENILYLVNSVSINKKQDEERCPIFKVTDIPQYAEAGIDRIGE |
| | | | | | | CKYVIKLKLFTVGGRYGTTYMPEAFSGLYDRRTNQRYDLIDRACKGSYVYLPIY |
| | | | | | | ISDKDSRKWSDRLKNTFTMSAFSISERKTKKDGRVLCVNLSYTHIQHKPRNYFK |
| | | | | | | VYPTYGVDINVKHSLFSTNATNLNGYIDVNGELIDFYNSIGRPDKLKNINLAVNL |
| | | | | | | GDKGSTAGNHTSFLLVSDLGDWDDHGKKRNDRFVKREDGTYYSRPDLLADGL |
| | | | | | | NFLKKKYSGDRKSYDYINTTIAFRNNQTRLRCIDKEWNDRKRAYDEEHGKPEK |
| | | | | | | GERSGFYPTEDAERLTMERQALRREIAENTRQVIAYMLWLFQENGIKSIALEHLD |
| | | | | | | CGSWKNFRDQLKPKWFCNNCKYWAEKYGIPYEATKYYRSNRKYFDIEDGELIP |
| | | | | | | ENVKLNALGEAENDKRYFQTSIAKAAGMSSIKQMFSIVSQNYDIQVNYTDQAYT |
| | | | | | | SQTDSRNGTVDKDFRVSQECYIGSDGTKMNCDFNAAVNIADRISDDTLREEFNK |
| | | | | | | ELPMKEFVKYGATQIVSRNRRWNAENAERVTMAAFQKRKNADGGAHTAAVKP |
| | | | | | | TSKTEESSLKLVAE* |
| MG191 effectors | 1081 | MG191-17 | pR0tein | Unknown | uncultivated | MAKHKKIEANEIVKTFSLKVKNPNSISLDVFPEYQRYYNCVSDWIKDNLLLKIGD |
| | | cffcctor | | | organism | LYQYIPLKKRETEYAKVLISDEWRGKPLFMMFMKGYSSNNRDNALYEAINACN |
| | | | | | | PENYTGNILGISDTYYRRFGYIGSVISNYAAKISKMNTGVKKHTLPENPDDVILM |
| | | | | | | EQTIYEMSHNGWTSLKDWEGHMEYLDSKMDSNTIYKQRITALYDFYKSHINEV |
| | | | | | | NSKMETMTIDNI,VKFGGCRRKDSKKSIYIGGPSGFPLTIKQIDGNSFNIKFVNNINI |
| | | | | | | DVYGRRDVIKDGVLLYDIINKHGNSFIIKIKNGEMYIDTTITVPFNKKIATTKKVV |
| | | | | | | GVDVNVKHMLLATSINDGNLKGYVNIYKEVISDPDFKKVCNGDLMRHFTNFSE |
| | | | | | | FITFCPLEFDLLFSRVCKQKDVFDENSVIEEAFSNVLDRLKWRFFNSGDNVNRIY [symbol] |
| | | | | | | ENVKKLRSQLKSYAILKNAYYKKQSEYDLGKSEEFIQEHPFADTDTGKEMLSKN [symbol] [symbol] |
| | | | | | | KRVGDRIIGCRNNIIQYAYGVFKTNGYDMISLENLSRNGFEKTKTFPTVTSIMKK |
| | | | | | | YKILGCTKEEMEANDVYSVIQKGYYDIIFDNGKVSDVKLSQKGELQKIKDDFFD [symbol] |
| | | | | | | LLMKVIHFADIKDYFITLSNNGTVGVSLVPSEFTSQMDSIEHKIYCSNDKYGNLK [symbol] |
| | | | | | | LVNKHRVRISQERHINGLNADYNAANNIAHIVSDDKFRNWFMRQSRTDKPLYN [symbol] |
| | | | | | | KPSYESTVKSPSIVVSKLKKEGMVKVMEA* |
| MG191 effectors | 1082 | MG191-18 | pR0tein | unknown | uncultivated | MHLKKSISEDEITKTFVLKVNCGKDDIISLWKPAANEFSNYYNKLSKWIGSNLTS |
| | | effector | | | organism | MKIGDLAKYIDNKENEYYKAVTDESKKDLPLYKIFKGFSSVYADNAMYCMVKI |
| | | | | | | ENPEEYNKNILCISESNYRKNGYFQCVMSNFRTKMSSLKTGIKQKKIDPNNVDEI |
| | | | | | | TLKLQTAYEVSRYGIEKTKEFEDLVKSLKTREETPQLKERIERLSILMEYYKNNE |
| | | | | | | DAVKDYIDEKIAENLQKFGCCIRKSLNSISIFRQNAKMTKDGNTSFNLELIFNKKP |
| | | | | | | YIINMLGNRQIVRYANGQRNDIIDITEKHGCLFTFTIKENELFTHITTSVKFNKNEQ |
| | | | | | | EIRNAVGVDVNIKHMLLATSIVDDGKVKGYVNLYRELCNDKEFKESFKSNDEFE |
| | | | | | | AYRQISETVNFGVLETESLFGRVIEQQKGIGSDSNLIKRESAIRNVFDNILKTTKDR |
| | | | | | | RIVDYINYVKMLRAKYKAYFILKEKYYERQKEYDLMMGFTDESTESKESMDAR |
| | | | | | | RSEFPFANTREAQEILTKLQTVGQDIIGCRDNIVTYAFNIFKDNGFDTISLEYLDSS |
| | | | | | | QFTKMKMPTPLSLLKYHKLEGKTEKEVETIINEKKFHKSHYKIRYNNETKLVDNI |
| | | | | | | EYSNEGIWKQKKLNFMNLIIKAVGFADIKDKFVQLCNNNIIMNTVFCPSAFTSQM |
| | | | | | | DSITHALYFVPKITKDKKGREIQKFVLANKNTVRSTQERHINGLNADYNSARNL |
| | | | | | | KYIAENETLRNDMTDHKTMYGVPTWNIKKSFKKCLSAKTIETFRNRGNVVYGY |
| | | | | | | VNENGEFVKM* |
| MG191 effectors | 1083 | MG191-19 | pR0tein | Unknown | uncultivated | MANKKEGVANKTYCFKVKRMYDMNLETIKKTIDEFDYGYNLCSEALHKILTEK |
| | | effector | | | organism | TIVLDSYIPESSKDKARSKYIKDKEIEENGGKKASKGFPLLVTEDYADKPIYAFIEE |
| | | | | | | VDANKVDNALYWYFKKALNGYGETNSLHINESTFRRNGFCKQVGGNYRTKLQ |
| | | | | | | QFNNLGIQKIKKVDITSESPYEVILEQCARDVMCYASREHLKNISDFNTKTFAKTL |
| | | | | | | SDKKASGDKVQKKELERCELMCKFFKEHETDVKRKIEKLTVEIMPVCSHDLNSE |
| | | | | | | RKVMSVIWGSTSTTKDTAIYDANGKQIGFRKGNLTITQISKYDFNFRFDPSSGHG |
| | | | | | | FSCDVWGRKDVIKGDDVLVVLYEAMSNNTLFKIVDNTMYIHLSCSAPFKTKTTP |
| | | | | | | IPKMADEKKVGCDVNTKHAFLQFDEGVNVDDLKGYVNIYQALIEDKAYNELCK |
| | | | | | | KYPTEGNKSLYETLSKHACFCPIESQWLMYRAIPRKDENGKEFKDDSRNPIANKF |
| | | | | | | EEVFSQVIDRLLDEAKTKGEHEKVYYLGCVRKFR0KLLSVCRIQTAYDYSGTLY |
| | | | | | | AIGVNKAFPDSEQAKKELASHPFNTTEKGKEMLQKKENIRKRIISCRNNIIHYAYS |
| | | | | | | VLAKNGYGCIALENLTTAGLEKKGSDYMKYPETFLYSKGFLGMTKEEIQKDQY |
| | | | | | | LKKLYDEKMYKYDTDKEGRIENILFTKEGKQRKEKSDFNNFALKMVHFASVKD |
| | | | | | | YFVTLQNKGTVAVAWVPSEYSSQIDSKEHILYVEEKKKGKRIVPVKKELVRPNC |
| | | | | | | EDYFINGLNADINAANNIRYYVLNNSKFGNFVQCIDVEFNKPSCSPTKGDIVKKI |
| | | | | | | QDSEQYRTLNQDFS* |
| MG191 effectors | 1084 | MG191-20 | pR0tein | unknown | uncultivated | MANKKEGVANKTYCFKVKRMYDMNLETIKKTIDEFDYGYNLCSEALHKILTEK |
| | | effector | | | organism | TIVLDSYIPESSKDKARSKYIKDKEIEENGGKKASKGFPLLVTEDYADKPIYAFIE [symbol] |
| | | | | | | VDANKVDNALYWYFKKALNGYGETNSLHINESTFRRNGFCKQVGGNYRTKLQ [symbol] |
| | | | | | | QFNNLGIQKIKKVDITSESPYEVILEQCARDVMCYASREHLKNISDFNTKTFAK [symbol] |
| | | | | | | SDKKASGDKVQKKELERCELMCKFFKEHETDVKRKIEKLTVEIMPVCSHDLNS [symbol] |
| | | | | | | RKVMSVIWGSTSTTKDTAIYDANGKQIGFRKGNLTITQISKYDFNFRFDPSSGHG [symbol] |
| | | | | | | FSCDVWGRKDIIKGDDVLVVLYEAMSNNTLFKIVDNTMYIHLSCSAPFKTKTTP [symbol] |
| | | | | | | PKMADEKKVGCDVNTKHAFLQFDEGVNVDDLKGYVNIYQALIEDKAYNELCK [symbol] |
| | | | | | | KYPTEGNKSLYETLSKHACFCPIESQWLMYRAIPRKDENGKEFKDDSRNPIANKF |
| | | | | | | EEVFSQVIDRLLDEAKTKGEHEKVYYLGCVRKFR0KLLSVCRIQTAYDYSGTLY |
| | | | | | | AIGVNKAFPDSEQAKKELASHPFNTTEKGKEMLQKKENIRKRIISCRNNIIHYAYS |
| | | | | | | VLAKNGYGCIALENLTTAGLEKKGSDYMKYPETFLYSKGFLGMTKEEIQKDQY |
| | | | | | | LKKLYDEKMYKYDTDKEGRIENILFTKEGKQRKEKSDFNNFALKMVHFASVKD |
| | | | | | | YFVTLONKGTVAVAWVPSEYSSQIDSKEHILYVEEKKKGKRIVPVKKELVRPNQ |
| | | | | | | EDYFINGLNADINAANNIRYYVLNNSKFGNFVQCIDVEFNKPSCSPTKGDIVKKI |
| | | | | | | QDSEQFRTLNQDFS* |
| MG191 effectors | 1085 | MG191-21 | pR0tein | Unknown | uncultivated | MAQHKSNNEESAINKTFIFKAKCDKNDVISLWEPAAKEYGDYYNEVSKWIADN |
| | | effector | | | organism | LITMKIGDLAQYITNKKSAYYIAVTAETKKDLPLYRIFQKGFSSQCADNALYCAI |
| | | | | | | KSINPENYKGNSLGIGESDYRRFGYIQSVVSNFRTKMSSLKATVKWKKFDVNNV |
| | | | | | | DDETLKIQTIYDVDKYGIETAKEFKELIETLKTRVETPQLNDTIARLECLCDYYSK |
| | | | | | | NEKAINNEIETMAIADLQKFGGCQRKSLNAFTIHKQDSLMEKVGNTSFRLQLPFR |
| | | | | | | KKTYVINLLGNRQVVNFVNGKRVDLIDIAENHGDLVTFNIKNGVLFVHLTSPIVF |
| | | | | | | DKDVRDIRNVVGIDVNIKHSMLATSIKDVGNVKGYINLYKELLNDDEFVSTCNE |
| | | | | | | SELALYRQMSENVNFGILETDSLFERIVNQSKGGCLKNKLIRRELAMQKVFERIT |
| | | | | | | KTNKDQNIVDYVNYVKMMRAKCKASYILKEKYYEKQKEYDVKMGFTDESTES |
| | | | | | | KETMDKRREEFPFVNTDTAKELLVKQNNIRQDIIGCRDNIVTYAFNVFKNNEYD |
| | | | | | | TLSVEYLDSSQFDKRRIPTPKSLLKYHKFEGKTKDEVENMMKSEKLSNAYYTFK |
| | | | | | | YENDVVSDIGYSDEGNLRRSKLNFGNWIIKAIHFADIKDKFVQLSNNNKMNIVFC |
| | | | | | | PSAFSSQMDSITHTLYYVEKITKNKKGKEKKKYVLANKKMVRTQQETHINGLN |
| | | | | | | ADYNSACNLKYIALNYELRDKMTDRFKASKKIKTMYNIPAYNIKSNFKKNLSAK |
| | | | | | | TIQTFRELGHYRDGKINEDGMFVEILE* |
| MG191 effectors | 1086 | MG191-22 | pR0tein | unknown | uncultivated | MANKKEGVANKTYCFKVKRMYDMNLETIKKTIDEFDNGYNICSEALHKILTEK |
| | | effector | | | organism | TIVLDSYIPESSKEKARSKYIKDKEIEENGGKKASKGFPLLVTEDYADKPIYAFIEE |
| | | | | | | VDANKVDNALYWYFKKALNGYGETNSLHINESTFRRNGFCKQVGGNYRTKLQ |
| | | | | | | QFNNLGTQKIKKVDITSKSPYEVILEQCARDVMCYASREHLKNISDENTKTFAK [symbol] |
| | | | | | | LSDKKASGDNVQKKELERCELMCKFFKEHETDVKRKIEELTVEIMPVCSHDLNS [symbol] |
| | | | | | | ERKVMSVIWGSTSTTKDTAIYDANGKQIGFRKGNLTITQISKYDFNFRFDPSSG [symbol] |
| | | | | | | GFSCDVWGRKDIIKGDDVLVVLYEAMSNNTLFKIVDNTMYIHLSCSAPFKTKTT [symbol] |
| | | | | | | PIPKMADEKKVGCDVNTKHAFLQFDEGVNVDDLKGYVNIYQALIEDKAYNEL [symbol] |
| | | | | | | KKYPTEGNKSLYETLSKHACFCPIESQWLMYRAIPRKDENGKEFKDDSRNPIAN |
| | | | | | | KFEEVFSQVIDRLLDEAKTKGEHEKVYYLGCVRKFRQKLLSVCRIQTAYDYSG [symbol] |
| | | | | | | LYAIGVNKAFPDSEQAKKELASHPFNTTEKGKEMLQKKENIRKRIISCRNNIIHY [symbol] |
| | | | | | | YSVLAKNGYDCIALENLTTAGLEKKGSDYMKYPETFLYSKGFLGMTKEEIQKD |
| | | | | | | QYLKKLYDEKMYKYDTDKEGRIENILFTEKGKKDKEKSDENNFALKMVHFAS [symbol] |
| | | | | | | KDYFVTLQNKGTVAVAWVPSEYSSQIDSKEHVLYVKDKKKNGKIVPVKKEAVR |
| | | | | | | KKQEDYFINGLNADINAANNIRYYVLNNSKFGKFVQCIDVEFNKPSCSPTKGDIV |
| | | | | | | KKIQDSEQYRILNQDFS* |
| MG191 effectors | 1087 | MG191-23 | pR0tein | Unknown | uncultivated | MEETKEIAVETPVVESAVDVVEKPKEKRFKRDMTNLTHKTESLKVIALYGVTVF |
| | | effector | | | organism | DLRKLGVQYFGFYNGCSDLINANLTTMKIGDLYEYIPDKSKGKDFAQLLISDEW |
| | | | | | | KDKPLYKIFSKKYFTQGADMALEYFYKANVEIPETEIHTTYSSFWLPCMHSLNA |
| | | | | | | NYRTKLQQFDRVNITKDDITEDSEHDVLLNQCVRDVSKYKITKVSEYEDFIKSCK |
| | | | | | | KKEKVSQDFINREILLCKFYKEHTDEVNDTIEKIMCENLQKMGGCHREYDDSFL |
| | | | | | | TMLCEFRGTSIKGVGRQNSMDITKIGKYIYHIDFKNMFSVDVYSRRDIDNGDETL |
| | | | | | | IDITDKTLVRSSVVFKIQNDELYINLQCIVPFKGKVKPIEEIDDSKKIGCDVNIKHM |
| | | | | | | MLMFSENIKNLKGIPNIYAELVKNKEFKSLCTDEEYKYYVEFSKWVCVCPIELPL |
| | | | | | | LINRGKDNREGRMEAILSNVIRDMAEKANLTNHKESIYLYCVLKIRQQLFAAYSI |
| | | | | | | RNAFNRLNGVYTYNIYQKYDNDAAKEYILAHPFGKTEQGKQLLTKRRRNONTI |
| | | | | | | DSCRENIQQFTYSILEKCGYENLVLENLTDSGIKNEYKTRIPFPESLLGSKKYYGM |
| | | | | | | SEEEVLQQRNFWLIKDGYMTLSWDDDNCVDGAALTEKGKMTKMRNDFHDIVC |
| | | | | | | KGIHLAGIKDYFIMLQNNGSVSVGLVNNEYTSQMDSIEHKMYFKDKNTLCDKK |
| | | | | | | DLRKTQERHINGKLADLNAANNIEYFASKSCFENFLKPKKFAYCTPAYTPIKTGN |
| | | | | | | DYMRRQFKKFDMQHIVGK* |
| MG191 effectors | 1088 | MG191-24 | pR0tein | Unknown | uncultivated | MATHKKTDNQTIVKAYVMKAKMSKHDIERVWKPTIDEYIEYYNKLSDWICKNL |
| | | effector | | | organism | TSVTIGDLLKYVGEKQINKGVGYYTYFIDEQKTDLPLYTLFTDCPKTHADNLLFE |
| | | | | | | AVRKINPENYIGNLLSLFETGYRRNGYFDNVISNYRTKMTTLKINPKYKRFSSEN |
| | | | | | | MPTDEVLLEQTIYEVTKNDFKNDDDWKKSIDYMKQKSEPNTALIFRMETLFDY |
| | | | | | | WKDHKQDVEQYINQKRVECLKDFGGCKRRADGLSMVIFLNKKLTKIEADGLTS |
| | | | | | | YKITTNLFGGKYTINIFGHRALVSVCNGERVENENIDICNKHGERFTFKIENGNLF |
| | | | | | | VALTADYNYEKQPNLPKHIVGVDTNIKHSMLNSSIEDKGKVKGYVNLYKEFLSD |
| | | | | | | ENFKKTITSVKERKQYEELSKYATFGITELDSLFARATDTEKNILCKRELAMQDV |
| | | | | | | FEKLEKRYKDDHKIKFYLGSTQKLRAQYISYFKIKEAYNRKQQEYDLAHGKTD [symbol] |
| | | | | | | PDEVYKSDFINEPSAKEMLVKLNRIERKIIGCRNNIVTYAFSVIKDNGYDTIGVEY [symbol] |
| | | | | | | LTSSQFEKKRRLPSIKSLLNYHKLLGKPKDEWNLKEWNDVYVCYRPELDDAGN [symbol] |
| | | | | | | MNFTITNEGIKRNKESTFYNSFIKAIHFADVKDKFAQLTNNNTMNTVFIPSSFTSC [symbol] |
| | | | | | | IDSKTQKLYLLEYTEKCDNGKTKKVVKFINKRVLRKIQEQHLNGMNADNNAA [symbol] |
| | | | | | | NIRDITKNLLDVFTKKQADKNCYNSAEFMIQTKFKKRLSQATVFGELNRNGYV [symbol] |
| | | | | | | VLTKEEYDELTKSAK* |
| MG191 effectors | 1089 | MG191-25 | pR0tein | unknown | uncultivated | MAHKTNNGENTINKTFIFKAKCEKNDIISLWKPAAEEYCNYYNKLSKWIADNLI |
| | | effector | | | organism | TMKIGDLAQYITNONSAYYIAVIDESKKNLPLYKIFQKGFSSLCADNALYCAIKA |
| | | | | | | NPENYNGNILGICPSDYRGSGYVQSVISNFRTKMSSLKVTTKWKKFDVNNVDDI |
| | | | | | | TLKIQTICDIDKYGIESIKDFKKFIEVLKMREETPQLNDKIARLECLCGYYSKNEK |
| | | | | | | TINNEIETMAIADLQKFDGCQRKSLNTLTIHKQNSPIEKIGNTSFNLKLIFNKKPYII |
| | | | | | | NLLGNRQVVKFINGKRVDLIDITKKHGDTITFNIKNSELFVHLTVPIEFEKKVYDI |
| | | | | | | KNAVGIDVNIKHMLLSTSIIDNGTVKGYLSIYKELLKDSDFVSTLNDSEYEMYSK |
| | | | | | | MSKNVNFGILEVNSLFERVVNQLHDNDLNNKLIRREEAIMKRLNNIIKSCKDQRII |
| | | | | | | NYINSVKMMRAKYKAYYILKKKRDELQSEYDNMMGFGDSDDPRRKDFQFIDTL |
| | | | | | | EFQKITSKIHRVKQDIIGCRDNIITYAFNIFMNNKYDTISLEYLDNSQFDRWRFTSP |
| | | | | | | KSLLDYYHFRGHTEDEVNQLIKEKNIKKEYYIFKYDNNVLVDIELSKNGEKNLQ |
| | | | | | | KSRFVNNIIKAIQFADIKDKFVQLTNNNDMNVVFCPAAFTSQADSKTIICLYFVK |
| | | | | | | GKNNKYVLANSKSVRTQQETHINGENADWNAACNLKYNAENTTFLEMTTKKT |
| | | | | | | KDGKSMYNTPGYNIQKKFKKNVSAVTLNTIKKIGNIKYGNIINGQFIEDK* |
| MG191 effectors | 1090 | MG191-26 | pR0tein | Unknown | uncultivated | MAKHKKTESTQIVKTFSFKVKNANELSLNTLNKAITEYQIYYNYCSDWIKDRLT |
| | | effector | | | organism | MKIGDLYELIPMNKRASGYAMLLISEEWKDKPLYMMFKKSFPGNHRDNAIYEAI |
| | | | | | | TTCRPEHYTGNILNFSDTYYRRFGYIASVISNYVTKISKMSTGIKRKNLSNDSDAD |
| | | | | | | TLIGQVIYEMEHNGWTSVKDWENQIEYLESKTDTNPNYVSRITTLYEFYKSHVD |
| | | | | | | EITLKMEAMSIDSLIKFGGCRRKESKKSMCIMGASNAPFDITQIEGNFFNIKFSKN |
| | | | | | | LNLDVFGRRDVIKDGVLLVDIINEHGNSFVLKIINNDIYIDINTSVSFDKEITTFNN |
| | | | | | | VVGVDVNIKHMLLATSILANEGIKGYVNIYKEAINDSGFKKECNSTVMKYFTDL |
| | | | | | | SKFVTFCPLEFEFLFSRVCKQKGIYNENSAMEEAFSNVLNKLKWEFIEKGDNTKR |
| | | | | | | IYVENVMKLRSQMKAYAIVKNAYYKQQSEYDLGKSEEFIQEHPFSNTDKGVEIL |
| | | | | | | HKLDNISIKILGCRNNIIQYAYNLFEINGYDMIALEKLTSSQFEKVKTFPTVNSLLK |
| | | | | | | YHKILGCTQEKMEQKDIYSVIKKGYYDIIFNDDVVSDAKLSTKGELSKFKDDFFN |
| | | | | | | LMIKSIHFADIKDYFITLSNNGTVGVSLVPSFFTSQMDSIDHKIYFVQDNKSGKMI |
| | | | | | | LANKHKVRNSQEKHINELNADYNAAKNIAYIMEDATCKNMFMKQSRTDKSLY |
| | | | | | | NKPSYETFVKTQGSVVAKFKKAGMVKLLA* |
| MG191 erRNA | 1091 | MG191-27 | nucleotide | artificial | | UAUUGUGUUGGGAUACACUUUUAUAGGUAUUUACAAC |
| sequences | | crRNA | | sequence | | |
| | | sequence | | | | |
| MG191 crRNA | 1092 | MG191-30 | nucleotide | artificial | | AUUGUUGUAAUAUCUAUUUUGUAAGGUGUAAACAAC |
| sequences | | crRNA | | sequence | | |
| | | sequence | | | | |
| MG191 crRNA | 1093 | MG191-31 | nucleotide | artificial | | GAUGUUGUUAUAUCUUUUUGUAGAUAAUAAACAAC |
| sequences | | crRNA | | sequence | | |
| | | sequence | | | | |
| MG191 crRNA | 1094 | MG191-1 | nucleotide | artificial | | GUUGUUUAAUACCUAUAAAAGAGUAUAUACAACAAG |
| sequences | | crRNA | | sequence | | |
| | | sequence | | | | |
| MG191 crRNA | 1095 | MG191-2 | nucleotide | artificial | | CUUGUUGUAUAUAUUCUUUUAUAGAUAUUAAACAAC |
| sequences | | crRNA | | sequence | | |
| | | sequence | | | | |
| MG191 crRNA | 1096 | MG191-3 | nucleotide | artificial | | CUUGUUGUAUAUACUCUUUUAUAGGUAUUAAACAAC |
| sequences | | crRNA | | sequence | | |
| | | sequence | | | | |
| MG191 erRNA | 1097 | MG191-4 | nucleotide | artificial | | AUUGUUGUAAUACACUUUUUAUAAGGUAUGAACAAC |
| sequences | | crRNA | | sequence | | |
| | | sequence | | | | |
| MG191 crRNA | 1098 | MG191-5 | nucleotide | artificial | | CUUGUUGUAUAUAUUCUUUUAUAGGUAUUAGACAAC |
| sequences | | crRNA | | sequence | | |
| | | sequence | | | | |
| MG191 crRNA | 1099 | MG191-6 | nucleotide | artificial | | AUUGUUGUAGAUACCUUUUUAUAAGGUUUGAACAAC |
| sequences | | crRNA | | sequence | | |
| | | sequence | | | | |
| MG191 crRNA | 1100 | MG191-7, | nucleotide | artificial | | AUUGUUGUAACUCUUAUUUUGUAUGGAGUAAACAAC |
| sequences | | MG191-8, | | sequence | | |
| | | MG191-9, | | | | |
| | | MG191-11 | | | | |
| | | MG191-12 | | | | |
| | | and MG191- | | | | |
| | | 14 effectors | | | | |
| | | crRNA | | | | |
| | | sequence | | | | |
| MG191 crRNA | 1101 | MG191-10 | nucleotide | artificial | | AUUGUUGUAACUCUUAUUUUGUAUAGAGUAAACAAC |
| sequences | | crRNA | | sequence | | |
| | | sequence | | | | |
| MG191 erRNA | 1102 | MG191-13 | nucleotide | artificial | | AUUGUUGUAGACACCUUUUUAUAAGGAUUGAACAAC |
| sequences | | crRNA | | sequence | | |
| | | sequence | | | | |
| MG191 crRNA | 1103 | MG191-15 | nucleotide | artificial | | AUUGUUGAAUAAUUCCUUUUAUAGGGUUGUUACAAC |
| sequences | | crRNA | | sequence | | |
| | | sequence | | | | |
| MG191 crRNA | 1104 | MG191-16 | nucleotide | artificial | | AGUGGAUGCGGUAUCAACUUGAUUGGUACUGACACC |
| sequences | | crRNA | | sequence | | |
| | | sequence | | | | |
| MG191 crRNA | 1105 | MG191-17 | nucleotide | artificial | | AUUGUUGUAAUCCACAUUUUGUAUGGGAUUAACAAC |
| sequences | | crRNA | | sequence | | |
| | | sequence | | | | |
| MG191 crRNA | 1106 | MG191-18 | nucleotide | artificial | | AUUGUUGGAAUACAUCUUUUUUAGGGUAUUUACAAC |
| sequences | | crRNA | | sequence | | |
| | | sequence | | | | |
| MG191 erRNA | 1107 | MG191-19 | nucleotide | artificial | | AUUGUUGGUUAUCCUAAUUUCAUAGGAAUACACAAC |
| sequences | | and MG191- | | sequence | | |
| | | 22 crRNA | | | | |
| | | sequence | | | | |
| MG191 erRNA | 1108 | MG191-20 | nucleotide | artificial | | AUUGUUGGUUAUCCUAAUUUUAUAGGAAUACACAAC |
| sequences | | crRNA | | sequence | | |
| | | sequence | | | | |
| MG191 crRNA | 1109 | MG191-21 | nucleotide | artificial | | UAUUGUUGGAAUAUCACUUUUGUAGGGUAUUCACAAC |
| sequences | | crRNA | | sequence | | |
| | | sequence | | | | |
| MG191 crRNA | 1110 | MG191-23 | nucleotide | artificial | | AUUGUUGUUACAUCAUUUUUAUAAGGUGUAAACAAC |
| sequences | | crRNA | | sequence | | |
| | | sequence | | | | |
| MG191 crRNA | 1111 | MG191-24 | nucleotide | artificial | | AUUGUUGAAUGGCUAUGUUUGUAUGCUAUUUACAAC |
| sequences | | crRNA | | sequence | | |
| | | sequence | | | | |
| MG191 crRNA | 1112 | MG191-25 | nucleotide | artificial | | AUUGGUGGAGUUCGCAUUCUAUAUCCUACUUACACC |
| sequences | | crRNA | | sequence | | |
| | | sequence | | | | |
| MG191 erRNA | 1113 | MG191-26 | nucleotide | artificial | | AAUGUUGUAAGCCUCUAUUUGUAGGGCGUUAACAAC |
| sequences | | crRNA | | sequence | | |
| | | sequence | | | | |
| MG191 effectors | 1114 | MG191-27 | pR0tein | unknown | uncultivated | MANKRTDTTINLNKTVIMLTNMLPEVRAMFQAGIRQAQAYADLVNKWICSNLT |
| | | effector | | | organism | NKIGEVLLPYIDNKNCVYYELCYKYKEAPLYTIFMKGKFDLNSRNNALYCAVV |
| | | | | | | AQNIDNYSGNIFGFSQSDYRRNGYCKVVFSNYATKMSSLKPSIKKVTINEESTEE |
| | | | | | | TIQSQVIYEMFTNGRQWGKPEYFAEHLKYLEMKDNVSDKLMFRMKTLCEYYQ [symbol] |
| | | | | | | HTDLIDTMAMNAGVEALKQFEGLKLNRDKFSMTITTNSTSPYTLTRVAGTCAY [symbol] |
| | | | | | | LHIPCRKRSYDIRLWGNRQTVRWVNGELVDIADIINQHGQTIIFTIKNGNVYVHIP |
| | | | | | | YGLNFEKTEHEIKNVVGVDVNTKHMLMQTSIKDNGWVKGYVNIYKALVEDEE |
| | | | | | | FVKYISKSDLKLYKDLSKYVSFCPLELNLLYTRYLSKKGLPFNEADNNAEKCVE |
| | | | | | | KVLNNLVKQYEGDDVHVVNYIHNVKKLRALCKASFVLYKKYAELQKAFDDAQ |
| | | | | | | GYNDQSTETKETMDKRRWENPFIQTREAQELIAKMDNAVAGIIGCRDNIITYAY |
| | | | | | | KVFGDNNYDTVGLENLTTSQFDNYSTVKSPKSLLSYYGLLGQQVDSDKYNAVM |
| | | | | | | TESNKDWYDFKTDGDGNITDITLTAAGEAQKAKSLFNNKVLKNIHFADVKDKFI |
| | | | | | | QLGNNGSIQTVLVPPSYTSQMDSKTHTIYVKETVDPKNKNKKKLKLVDKKLVR |
| | | | | | | IIGQEYIIKNGLNADINAALNIAYIVENQEMREVMCLHIPSKKDGVYDQPFLKATT |
| | | | | | | KYPATVAGILLKMGKTTNWGEK* |
| MG191 effectors | 1115 | MG191-28 | pR0tein | Unknown | uncultivated | MGHVRLQKREGEVYKTYKLKVKKFEGNVDIIAGITEYDQKFNNVSQWIADHLT |
| | | effector | | | organism | TMTIGEAASRICQKKLQGKAKYVKHCLSAEWRDQPLYKIFCKGFNGMDADNLII |
| | | | | | | ECTKVEDNCRYEKEKSLGFTESTFRSFGFAADAVFNMKSRMTSAKVKVGRKNI |
| | | | | | | DDESSDEDKCMQTVYEIQKHDLLTDDNWKDWMSYLEMKSDQERELQRINILYD |
| | | | | | | YFRRNRQTVFDKLDNLKVEMLSKFSGSKRKSNRKTLTFNKLLYDIKKKDDCQG |
| | | | | | | FELSFAVGKEHVKFDLFGHRALIKNGEILTDIEKRHGSQLSIEIDGEDMYVIVSMR |
| | | | | | | DCCVKNESNLNKIIGADVNIKHAFLMTSEKDDGSLNGYVNLYKELLSDKEFIDV |
| | | | | | | LNKDEFDIFTELSKYVTFGLIETPYFGSRIIDTTQHDKIVEDRITSGMKKVASRLIS |
| | | | | | | EGKERERIYVQNVLKIRALLKSLFSVKLAYSIDQCVYDNLMKFGGNDPRRKEEG |
| | | | | | | FHTTCRGISLRSDIDLLSKKILACRDSIAEYCYYVLGLNGFDGIALENLESSSFMD |
| | | | | | | VKISYPTCKSMLEHYKLKGKTVDEAENDENVGKFVKQGHYDMITVYGKIVDM |
| | | | | | | KYSEKAVKLHRKNLLYDTVIKSTHFADVKDKFIELSNNGNVSVVIVPPYFSSQM |
| | | | | | | DSVTHKVFTEEIVVYRKDKNGKNRKNRKTVLVDKRKVRKTQESHINGLNADYN |
| | | | | | | AALNLKYIADNVDWRSTLCFKTWNTYGSPQWDSKIKNQKTMIDRLDSLGAIEL |
| | | | | | | KKW* |
| MG191 effectors | 1116 | MG191-29 | pR0tein | unknown | Uncultivated | MARNNKKLEGLVKSYKFQAKNVDIMIDILEEYSEYYNRVSNWINDNLMTLTIG [symbol] |
| | | effector | | | organism with | IALKCNTSQSVGYMQIALSDDYKQLPMYKLFEKKYPSTHCLNLLYAYITNNNID |
| | | | | | predicted | NYTGNIWGLSTTNYRKYGYINLVINNYRSKFANLNFNVKRSKIDINSNVEEIEK [symbol] |
| | | | | | taxonomy | CLYERIRLGLIKIEDWKKHIDSLKRQEIINEKALHRFNILSDYFNKNGESINERQSI |
| | | | | | Unclassified | LTIEQLQKFNGCQMKNKSMRIQVQNFKLNEKPNSLGYILQIHLNKKLFNIELLGN |
| | | | | | | RQIRKGNKDNYTTLVDIINNHGIDIIFKLENNKLYVILTSTDTTFNKTITTPNKICG |
| | | | | | | DINIKHALLNTSERDSMNIQGYVNLYKRVLENDNFVSLLSKEEYETFSNMASYL [symbol] |
| | | | | | | FCPIEFNYLYSRISNDEYTEKERLFSKILIDIQKEYENKKGCDLEKNYIHSVNKLR [symbol] |
| | | | | | | KIIAYHKLKTIYTQKKQEYDIKMGFVDDSTLSKDGMDKRRFESPFNNTDCAKQ [symbol] |
| | | | | | | KSKLDNVLQDIEGCRKNIITYVYKIFETNGYVTIGLENLDNSNFEYHKPLCSIKSL |
| | | | | | | LNYHKLLNKTIEQVKENNSVADLIEKDYYIFTFDENKNIIDAKFSDKGFYKINKS |
| | | | | | | WFINQLIKVLHFASIKNYFITLSNNGKTQIVLVPSEFTSQINSLTHKLPMIKNDKGE |
| | | | | | | NVRPTKFRVRKTQEKHYNGLNADINATLNIKYIVENEEWRNVLCKSPNKKEYK |
| | | | | | | DARGYNFPTLIATQKHYNVSKQLEKLGGIEFIQ* |
| MG191 effectors | 1117 | MG191-30 | pR0tein | Unknown | uncultivated | MAHKKQKEENEIIKTISLKVKDYAGYPIVEAMREYTKYYNKISQWINSNLLTINI |
| | | effector | | | organism | GELSAFMPDECKTHNYYTYMMSPDWVNEPLYKMFMKGFHTQHCDNILFNVVK |
| | | | | | | TLNIDEYTGNSLGLSASCFRRSGYFQNVVSNYKSKFANPHISIRRKNLSDLPTEDE |
| | | | | | | LLEQCIYEIQNGLSSKTKWEEQIEYLKERDDSKQIYLTRLNTLFTYYKANKDFVD |
| | | | | | | EQMQIKSVESLANFGGCVRKNDKLSMNLVFSSNSPYKVVLNEKRNGYILSYSNN |
| | | | | | | FSIELYGNRMGLLNGVEMFNIVDKHSNNITFKMDNDELFVNIPVSVNFVKKANE |
| | | | | | | TNKVVGVDVNLKHSIFATNIIDDGKLDGFVNIYRELLSDVDFVKSCHNDLLNFIL |
| | | | | | | DVEKYAFFMPLELGLLSSRVMNQCGYSTIGKYEKLFTTEEHFLRVLRQLEKRFQ |
| | | | | | | ESGENQKRIYIENVIKMRAQLKAYVTLKYAYNKANKDYDLKMGFVDESTANKE |
| | | | | | | TMDQRRFENQFVNTDTAQEILGKMRRIANVITSCRNNIICYMYKIFENNGYGVV |
| | | | | | | ALEKLQSSQMKKEKRIPSLLSLLKKHKVEGYTINELKDKSVFKFIERGYYTFDFD |
| | | | | | | DDNKITGVQFSDAGEAVNIETELHNLALKTIHFADAKDYFVTLSNNGSVSVALV |
| | | | | | | PSQFTSQMDSTKHVLYAKKNNKGKLGIVSEHEVRPKQERHINGLNGDYNAACNI |
| | | | | | | AYIFENDEWRNAFMKMNPNEYGKALFETNIESTSTIINTLKKINPDNIISFDECEK |
| | | | | | | TKKVAA* |
| MG191 effectors | 1118 | MG191-31 | pR0tein | Unknown | uncultivated | MAGHSKIKENHIMKAFLMKVKETRKNQWQSNFIRSEIAKFTNYYNGLSKFIADR |
| | | effector | | | organism | LLDDMVTTLAPLIEEKKRDSEYYKYLTNGDWDGKPLYFIFKEGFNSTNADNILA |
| | | | | | | NSLVRVYCEQNYTGNGFGLSYSYYVVIGFAKEVIANYRSSFQKPKVKIKKKKLS |
| | | | | | | ENPTEDELIEQCIYTIYYEFNEKKDIKKWKDEIKFLKERGESKETRLKRIQTLFEFY |
| | | | | | | KDKNHKELVDERVANLVVDNIKEFGGCKRDIGCPSMGIQIQHNFDISINEKRNGY |
| | | | | | | TICFGPNKKNITKLEVFGNRMVLINGEEIVDLPNTHGEKLTLIDRGNAIYAALTA |
| | | | | | | QVPFEKHMPDGNKTVGIDLNLKHSVFATSIVDNGKLAGYISIYKELLKDDEFVK |
| | | | | | | YCPKDLLRFMKDASKYVFFAPIEIELLRSRVIYNKGYACVENYENVYKAEVAFV |
| | | | | | | NVIKRLQSQCEANGDAQGALYMSYLSKMRAQLKNYINLKLAYYDHQSAYDLK |
| | | | | | | MGFNDISAESKETIDERRKLFPFSKEKEAQEILAKMKNISNVIIACRNNIAVYMY [symbol] |
| | | | | | | MFERNGYDFIGLEKLESSQMKKRQSRSFPTVKSLLNYHKLAGMTMDEIKKQEV |
| | | | | | | SNIKKGFYDLEFDADGKLYGAKYSNKGNVHFIEDEFYISGLKAIHFADMKDYF [symbol] |
| | | | | | | RLSNNGKVSVALVPPSFTSQMDSVEHKFFMKKNANGKLIVADKKDVRSCQEK [symbol] |
| | | | | | | KINGLNADYNAACNIGFIVEDDYMRESLLGSPTGGTYDTAYFDTKIQGSKGVYD |
| | | | | | | KIKENGETYIAVLSDDVITAEE* |
| MG191-28 erRNA | 1119 | MG191-28 | nucleotide | artificial | | AUUGUUGUAUGUGCCGUUAUAUACGGCAUUAACAAC |
| sequences | | crRNA | | sequence | | |
| | | sequence | | | | |
| MG191-29 crRNA | 1120 | MG191-29 | nucleotide | artificial | | CUUGUUGUAAUUACUCUUUUUUAGGUAAUAAACAAC |
| sequences | | crRNA | | sequence | | |
| | | sequence | | | | |
| MG119-2 sgRNA | 1121 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrCrGr |
| | | | | | | ArGrArUrGrCrArCrGrUrArArG*mA*mA*mA |
| MG119-2 sgRNA | 1122 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gB1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrGrAr |
| | | | | | | GrArUrGrCrArCrGrUrArArGrA*mA*mA*mU |
| MG119-2 sgRNA | 1123 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gC1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrUrAr |
| | | | | | | UrUrGrUrUrCrArArCrUrUrUrU*mA*mU*mU |
| MG119-2 sgRNA | 1124 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gD1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrArUr |
| | | | | | | UrGrUrUrCrArArCrUrUrUrUrA*mU*mU*mC |
| MG119-2 sgRNA | 1125 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gE1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArUrUr |
| | | | | | | GrUrUrCrArArCrUrUrUrUrArU*mU*mC*mU |
| MG119-2 sgRNA | 1126 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrUrGr |
| | | | | | | UrUrCrArArCrUrUrUrUrArUrU*mnC*mU*mA |
| MG119-2 sgRNA | 1127 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArCrUrUr |
| | | | | | | UrUrArUrUrCrUrArUrUrUrUrC*mC*mC*mA |
| MG119-2 sgRNA | 1128 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gH1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrUrCr |
| | | | | | | UrArUrUrUrUrCrCrCrArGrUrA*mA*mA*mA |
| MG119-2 sgRNA | 1129 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrCrUr |
| | | | | | | ArUrUrUrUrCrCrCrArGrUrArA*mA*mA*mU |
| MG119-2 sgRNA | 1130 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gB2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrUrUr |
| | | | | | | UrCrCrCrArGrUrArArArArUrA*mnA*mA*mG |
| MG119-2 sgRNA | 1131 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gC2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrCrAr |
| | | | | | | GrUrArArArArUrArArArGrUrU*mU*mU*mA |
| MG119-2 sgRNA | 1132 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gD2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrArGr |
| | | | | | | UrArArArArUrArArArGrUrUrU*mU*mA*mG |
| MG119-2 sgRNA | 1133 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gE2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArGrUr |
| | | | | | | ArArArArUrArArArGrUrUrUrU*mA*mG*mU |
| MG119-2 sgRNA | 1134 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrUrAr |
| | | | | | | ArArCrUrCrUrGrCrArUrCrUrU*mU*mA*mA |
| MG119-2 sgRNA | 1135 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrArAr |
| | | | | | | ArCrUrCrUrGrCrArUrCrUrUrU*mA*mA*mA |
| MG119-2 sgRNA | 1136 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gH2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArGrAr |
| | | | | | | ArUrUrArUrUrUrUrGrGrCrArU*mU*mU*mA |
| MG119-2 sgRNA | 1137 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrCrAr |
| | | | | | | UrUrUrArUrUrUrCrUrArArArA*mU*mG*mG |
| MG119-2 sgRNA | 1138 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gB3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrCrArUr |
| | | | | | | UrUrArUrUrUrCrUrArArArArU*mG*mG*mC |
| MG119-2 sgRNA | 1139 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gC3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrC |
| | | | | | | UrArArArArUrGrGrCrArUrArG*mU*mA*mU |
| MG119-2 sgRNA | 1140 | 119-2 hALB | Nucleotide | Artificial | | mU*mA*mC*rArCrArUrCrArUrUrArGrArUrGrUrGrArGrGrGrCrArArUrUrUrUrArU |
| targeting hALB | | gD3 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrUrArArA |
| | | | | | | ArUrGrGrCrArUrArGrUrArUrU*mU*mU*mG |
| MG119-2 sgRNA | 1141 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gE3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArArAr |
| | | | | | | UrGrGrCrArUrArGrUrArUrUrU*mU*mG*mU |
| MG119-2 sgRNA | 1142 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrArUr |
| | | | | | | UrUrGrUrGrArArGrUrCrUrUrA*mC*mA*mA |
| MG119-2 sgRNA | 1143 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArUrUr |
| | | | | | | UrGrUrGrArArGrUrCrUrUrArC*mA*mA*mG |
| MG119-2 sgRNA | 1144 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gH3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrArAr |
| | | | | | | GrUrCrUrUrArCrArArGrGrUrU*mnA*mU*mC |
| MG119-2 sgRNA | 1145 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArCrAr |
| | | | | | | UrCrCrUrArGrGrUrArArArArA*mA*mA*mA |
| MG119-2 sgRNA | 1146 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gB4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArCrC |
| | | | | | | UrUrUrUrUrUrUrUrUrUrUrUrU*mA*mC*mC |
| MG119-2 sgRNA | 1147 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gC4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrUrUrUrUrArCrCrUrArGrGrA*mU*mG*mU |
| MG119-2 sgRNA | 1148 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gD4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrUrUrUrArCrCrUrArGrGrArU*mG*mU*mU |
| MG119-2 sgRNA | 1149 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gE4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrUrUrArCrCrUrArGrGrArUrG*mU*mU*mU |
| MG119-2 sgRNA | 1150 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrUrArCrCrUrArGrGrArUrGrU*mU*mU*mG |
| MG119-2 sgRNA | 1151 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrArCrCrUrArGrGrArUrGrUrU*mnU*mG*mA |
| MG119-2 sgRNA | 1152 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gH4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | ArCrCrUrArGrGrArUrGrUrUrU*mG*mA*mA |
| MG119-2 sgRNA | 1153 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrA |
| | | | | | | CrCrUrArGrGrArUrGrUrUrUrG*mA*mA*mU |
| MG119-2 sgRNA | 1154 | 119-2 hALB | Nucleotide | Artificial | | mU*mA*mC*rArCrArUrCrArUrUrArGrArUrGrUrGrArGrGrGrCrArArUrUrUrUrArU |
| targeting hALB | | gB5 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrUrUrArC |
| | | | | | | CrUrArGrGrArUrGrUrUrUrGrA*mA*mU*mU |
| MG119-2 sgRNA | 1155 | 119-2 hALB | Nucleotide | Artificial | | mU*mA*mC*rArCrArUrCrArUrUrArGrArUrGrUrGrArGrGrGrCrArArUrUrUrUrArU |
| targeting hALB | | gC5 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrUrArCrCr |
| | | | | | | UrArGrGrArUrGrUrUrUrGrArA*mU*mU*mU |
| MG119-2 sgRNA | 1156 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gD5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArCrCrUr |
| | | | | | | ArGrGrArUrGrUrUrUrGrArArU*mU*mU*mU |
| MG119-2 sgRNA | 1157 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gE5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrAr |
| | | | | | | GrGrArUrGrUrUrUrGrArArUrU*mU*mU*mA |
| MG119-2 sgRNA | 1158 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArUrUr |
| | | | | | | UrUrArUrUrArArUrArArGrArU*mA*mA*mC |
| MG119-2 sgRNA | 1159 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrUrAr |
| | | | | | | ArUrArArGrArUrArArCrCrUrU*mG*mU*mA |
| MG119-2 sgRNA | 1160 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gH5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrArA |
| | | | | | | UrArArGrArUrArArCrCrUrUrG*mU*mA*mA |
| MG119-2 sgRNA | 1161 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArArAr |
| | | | | | | UrArCrArArArArUrArCrUrArU*mG*mC*mC |
| MG119-2 sgRNA | 1162 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gB6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrArAr |
| | | | | | | ArUrArArArUrGrCrCrArArArA*mU*mA*mA |
| MG119-2 sgRNA | 1163 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gC6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArArAr |
| | | | | | | UrArArArUrGrCrCrArArArArU*mA*mA*mU |
| MG119-2 sgRNA | 1164 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gD6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrArAr |
| | | | | | | ArGrArUrGrCrArGrArGrUrUrU*mA*mC*mU |
| MG119-2 sgRNA | 1165 | 119-2 hALB | Nucleotide | Artificial | | mU*mA*mC*ArCrArUrCrArUrUrArGrArUrGrUrGrArGrGrGrCrArArUrUrUrUrArUr |
| targeting hALB | | gE6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArGrAr |
| | | | | | | UrGrCrArGrArGrUrUrUrArCrU*mA*mA*mA |
| MG119-2 sgRNA | 1166 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gF6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrArAr |
| | | | | | | ArArCrUrUrUrArUrUrUrUrArC*mU*mG*mG |
| MG119-2 sgRNA | 1167 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gG6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | ArCrUrGrGrGrArArArArUrArG*mA*mA*mU |
| MG119-2 sgRNA | 1168 | 119-2 hALB | Nucleotide | Artificial | | mU*mA*mC*rArCrArUrCrArUrUrArGrArUrGrUrGrArGrGrGrCrArArUrUrUrUrArU |
| targeting hALB | | gH6 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrArCrUrG |
| | | | | | | GrGrArArArArUrArGrArArUrA*mA*mA*mA |
| MG119-2 sgRNA | 1169 | 119-2 hALB | Nucleotide | Artificial | | mU*mA*mC*rArCrArUrCrArUrUrArGrArUrGrUrGrArGrGrGrCrArArUrUrUrUrArUr |
| targeting hALB | | gA7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrGr |
| | | | | | | GrArArArArUrArGrArArUrArA*mA*mA*mG |
| DNA Sequence of | 1170 | 119-2 hALB | Nucleotide | Artificial | | GTCGAGATGCACGTAAGAAA |
| hALB Target Site | | gA1 | | sequence | | |
| DNA Sequence of | 1171 | 119-2 hALB | Nucleotide | Artificial | | TCGAGATGCACGTAAGAAAT |
| hALB Target Site | | gB1 | | sequence | | |
| DNA Sequence of | 1172 | 119-2 hALB | Nucleotide | Artificial | | TCTATTGTTCAACTTTTATT |
| hALB Target Site | | gC1 | | sequence | | |
| DNA Sequence of | 1173 | 119-2 hALB | Nucleotide | Artificial | | CTATTGTTCAACTTTTATTC |
| hALB Target Site | | gD1 | | sequence | | |
| DNA Sequence of | 1174 | 119-2 hALB | Nucleotide | Artificial | | TATTGTTCAACTTTTATTCT |
| hALB Target Site | | gE1 | | sequence | | |
| DNA Sequence of | 1175 | 119-2 hALB | Nucleotide | Artificial | | ATTGTTCAACTTTTATTCTA |
| hALB Target Site | | gF1 | | sequence | | |
| DNA Sequence of | 1176 | 119-2 hALB | Nucleotide | Artificial | | ACTTTTATTCTATTTTCCCA |
| hALB Target Site | | gG1 | | sequence | | |
| DNA Sequence of | 1177 | 119-2 hALB | Nucleotide | Artificial | | ATTCTATTTTCCCAGTAAAA |
| hALB Target Site | | gH1 | | sequence | | |
| DNA Sequence of | 1178 | 119-2 hALB | Nucleotide | Artificial | | TTCTATTTTCCCAGTAAAAT |
| hALB Target Site | | gA2 | | sequence | | |
| DNA Sequence of | 1179 | 119-2 hALB | Nucleotide | Artificial | | ATTTTCCCAGTAAAATAAAG |
| hALB Target Site | | gB2 | | sequence | | |
| DNA Sequence of | 1180 | 119-2 hALB | Nucleotide | Artificial | | CCCAGTAAAATAAAGTTTTA |
| hALB Target Site | | gC2 | | sequence | | |
| DNA Sequence of | 1181 | 119-2 hALB | Nucleotide | Artificial | | CCAGTAAAATAAAGTTTTAG |
| hALB Target Site | | gD2 | | sequence | | |
| DNA Sequence of | 1182 | 119-2 hALB | Nucleotide | Artificial | | CAGTAAAATAAAGTTTTAGT |
| hALB Target Site | | gE2 | | sequence | | |
| DNA Sequence of | 1183 | 119-2 hALB | Nucleotide | Artificial | | AGTAAACTCTGCATCTTTAA |
| hALB Target Site | | gF2 | | sequence | | |
| DNA Sequence of | 1184 | 119-2 hALB | Nucleotide | Artificial | | GTAAACTCTGCATCTTTAAA |
| hALB Target Site | | gG2 | | sequence | | |
| DNA Sequence of | 1185 | 119-2 hALB | Nucleotide | Artificial | | AAGAATTATTTTGGCATTTA |
| hALB Target Site | | gH2 | | sequence | | |
| DNA Sequence of | 1186 | 119-2 hALB | Nucleotide | Artificial | | GGCATTTATTTCTAAAATGG |
| hALB Target Site | | gA3 | | sequence | | |
| DNA Sequence of | 1187 | 119-2 hALB | Nucleotide | Artificial | | GCATTTATTTCTAAAATGGC |
| hALB Target Site | | gB3 | | sequence | | |
| DNA Sequence of | 1188 | 119-2 hALB | Nucleotide | Artificial | | TTTCTAAAATGGCATAGTAT |
| hALB Target Site | | gC3 | | sequence | | |
| DNA Sequence of | 1189 | 119-2 hALB | Nucleotide | Artificial | | TAAAATGGCATAGTATTTTG |
| hALB Target Site | | gD3 | | sequence | | |
| DNA Sequence of | 1190 | 119-2 hALB | Nucleotide | Artificial | | AAAATGGCATAGTATTTTGT |
| hALB Target Site | | gE3 | | sequence | | |
| DNA Sequence of | 1191 | 119-2 hALB | Nucleotide | Artificial | | GTATTTGTGAAGTCTTACAA |
| hALB Target Site | | gF3 | | sequence | | |
| DNA Sequence of | 1192 | 119-2 hALB | Nucleotide | Artificial | | TATTTGTGAAGTCTTACAAG |
| hALB Target Site | | gG3 | | sequence | | |
| DNA Sequence of | 1193 | 119-2 hALB | Nucleotide | Artificial | | TGAAGTCTTACAAGGTTATC |
| hALB Target Site | | gH3 | | sequence | | |
| DNA Sequence of | 1194 | 119-2 hALB | Nucleotide | Artificial | | AACATCCTAGGTAAAAAAAA |
| hALB Target Site | | gA4 | | sequence | | |
| DNA Sequence of | 1195 | 119-2 hALB | Nucleotide | Artificial | | GACCTTTTTTTTTTTTTACC |
| hALB Target Site | | gB4 | | sequence | | |
| DNA Sequence of | 1196 | 119-2 hALB | Nucleotide | Artificial | | TTTTTTTTTACCTAGGATGT |
| hALB Target Site | | gC4 | | sequence | | |
| DNA Sequence of | 1197 | 119-2 hALB | Nucleotide | Artificial | | TTTTTTTTACCTAGGATGTT |
| hALB Target Site | | gD4 | | sequence | | |
| DNA Sequence of | 1198 | 119-2 hALB | Nucleotide | Artificial | | TTTTTTTACCTAGGATGTTT |
| hALB Target Site | | gE4 | | sequence | | |
| DNA Sequence of | 1199 | 119-2 hALB | Nucleotide | Artificial | | TTTTTTACCTAGGATGTTTG |
| hALB Target Site | | gF4 | | sequence | | |
| DNA Sequence of | 1200 | 119-2 hALB | Nucleotide | Artificial | | TTTTTACCTAGGATGTTTGA |
| hALB Target Site | | gG4 | | sequence | | |
| DNA Sequence of | 1201 | 119-2 hALB | Nucleotide | Artificial | | TTTTACCTAGGATGTTTGAA |
| hALB Target Site | | gH4 | | sequence | | |
| DNA Sequence of | 1202 | 119-2 hALB | Nucleotide | Artificial | | TTTACCTAGGATGTTTGAAT |
| hALB Target Site | | gA5 | | sequence | | |
| DNA Sequence of | 1203 | 119-2 hALB | Nucleotide | Artificial | | TTACCTAGGATGTTTGAATT |
| hALB Target Site | | gB5 | | sequence | | |
| DNA Sequence of | 1204 | 119-2 hALB | Nucleotide | Artificial | | TACCTAGGATGTTTGAATTT |
| hALB Target Site | | gC5 | | sequence | | |
| DNA Sequence of | 1205 | 119-2 hALB | Nucleotide | Artificial | | ACCTAGGATGTTTGAATTTT |
| hALB Target Site | | gD5 | | sequence | | |
| DNA Sequence of | 1206 | 119-2 hALB | Nucleotide | Artificial | | CCTAGGATGTTTGAATTTTA |
| hALB Target Site | | gE5 | | sequence | | |
| DNA Sequence of | 1207 | 119-2 hALB | Nucleotide | Artificial | | AATTTTATTAATAAGATAAC |
| hALB Target Site | | gF5 | | sequence | | |
| DNA Sequence of | 1208 | 119-2 hALB | Nucleotide | Artificial | | ATTAATAAGATAACCTTGTA |
| hALB Target Site | | gG5 | | sequence | | |
| DNA Sequence of | 1209 | 119-2 hALB | Nucleotide | Artificial | | TTAATAAGATAACCTTGTAA |
| hALB Target Site | | gH5 | | sequence | | |
| DNA Sequence of | 1210 | 119-2 hALB | Nucleotide | Artificial | | CAAATACAAAATACTATGCC |
| hALB Target Site | | gA6 | | sequence | | |
| DNA Sequence of | 1211 | 119-2 hALB | Nucleotide | Artificial | | AGAAATAAATGCCAAAATAA |
| hALB Target Site | | gB6 | | sequence | | |
| DNA Sequence of | 1212 | 119-2 hALB | Nucleotide | Artificial | | GAAATAAATGCCAAAATAAT |
| hALB Target Site | | gC6 | | sequence | | |
| DNA Sequence of | 1213 | 119-2 hALB | Nucleotide | Artificial | | TTAAAGATGCAGAGTTTACT |
| hALB Target Site | | gD6 | | sequence | | |
| DNA Sequence of | 1214 | 119-2 hALB | Nucleotide | Artificial | | AAGATGCAGAGTTTACTAAA |
| hALB Target Site | | gE6 | | sequence | | |
| DNA Sequence of | 1215 | 119-2 hALB | Nucleotide | Artificial | | CTAAAACTTTATTTTACTGG |
| hALB Target Site | | gF6 | | sequence | | |
| DNA Sequence of | 1216 | 119-2 hALB | Nucleotide | Artificial | | TTTTACTGGGAAAATAGAAT |
| hALB Target Site | | gG6 | | sequence | | |
| DNA Sequence of | 1217 | 119-2 hALB | Nucleotide | Artificial | | ACTGGGAAAATAGAATAAAA |
| hALB Target Site | | gH6 | | sequence | | |
| DNA Sequence of | 1218 | 119-2 hALB | Nucleotide | Artificial | | CTGGGAAAATAGAATAAAAG |
| hALB Target Site | | gA7 | | sequence | | |
| MG119-2 sgRNA | 1219 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gA1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArGrUr |
| | | | | | | GrUrArCrUrCrCrUrCrGrArGrA*mG*mC*mG |
| MG119-2 sgRNA | 1220 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gB1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrGrCrUr |
| | | | | | | GrArGrCrGrUrGrCrUrCrArGrA*mU*mG*mC |
| MG119-2 sgRNA | 1221 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gC1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrUr |
| | | | | | | GrArGrArGrCrCrUrCrArArGrG*mC*mG*mC |
| MG119-2 sgRNA | 1222 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gD1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrUrGr |
| | | | | | | CrArGrCrUrCrGrUrGrCrArGrC*mU*mU*mC |
| MG119-2 sgRNA | 1223 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gE1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrCrGr |
| | | | | | | CrGrCrGrCrCrCrUrCrUrUrGrG*mA*mG*mC |
| MG119-2 sgRNA | 1224 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gF1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrCrG |
| | | | | | | GrUrArGrArGrCrUrCrCrArUrC*mU*mC*mC |
| MG119-2 sgRNA | 1225 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gG1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrUrGr |
| | | | | | | GrArArGrUrCrGrUrCrCrArGrG*mU*mA*mG |
| MG119-2 sgRNA | 1226 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gH1 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrArAr |
| | | | | | | GrUrCrGrUrCrCrArGrGrUrArG*mG*mG*mC |
| MG119-2 sgRNA | 1227 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gA2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrCr |
| | | | | | | CrUrCrCrArGrArUrCrCrUrUrG*mC*mU*mC |
| MG119-2 sgRNA | 1228 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gB2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrArGr |
| | | | | | | GrUrUrArUrCrCrCrArGrArArC*mU*mC*mC |
| MG119-2 sgRNA | 1229 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gC2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArGrGr |
| | | | | | | UrUrArUrCrCrCrArGrArArCrU*mC*mnC*mU |
| MG119-2 sgRNA | 1230 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gD2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrGrUr |
| | | | | | | UrArUrCrCrCrArGrArArCrUrC*mC*mU*mG |
| MG119-2 sgRNA | 1231 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gE2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrGrCrA |
| | | | | | | GrCrUrUrGrCrUrGrArArGrGrU*mG*mG*mA |
| MG119-2 sgRNA | 1232 | 119-2 | Nucleotide | Artificial | | mU*mA*mC*rArCrArUrCrArUrUrArGrArUrGrUrGrArGrGrGrCrArArUrUrUrUrArU |
| targeting hAPOA1 | | hAPOA1 | | sequence | | GrUrCrCrUrArArUrArUrUrGrArGrGrGrUrArUrUrArGrGrUrArArCrArArCrArCrUrU |
| | | gF2 | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrGrUrCrU |
| | | | | | | CrArCrCrCrUrCrCrArGrCrCrU*mA*mA*mA |
| MG119-2 sgRNA | 1233 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gG2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrCrArAr |
| | | | | | | GrCrUrGrCrGrCrGrArArCrArG*mC*mU*mC |
| MG119-2 sgRNA | 1234 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gH2 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrGrAr |
| | | | | | | UrArArCrCrUrGrGrArArArArG*mG*mA*mG |
| MG119-2 sgRNA | 1235 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gA3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrArAr |
| | | | | | | GrArArGrUrGrGrCrArGrGrArG*mG*mA*mG |
| MG119-2 sgRNA | 1236 | 119-2 | Nucleotide | Artificial | | mU*mA*mC*rArCrArUrCrArUrUrArGrArUrGrUrGrArGrGrGrCrArArUrUrUrUrArUr |
| targeting hAPOAl | | hAPOA1 | | sequence | | GrUrCrCrUrArArUrArUrUrGrArGrGrGrUrArUrUrArGrGrUrArArCrArArCrArCrUrUr |
| | | gB3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrGrUr |
| | | | | | | CrArGrCrUrUrCrCrUrGrArGrC*mG*mC*mU |
| MG119-2 sgRNA | 1237 | 119-2 | Nucleotide | Artificial | | mU*mA*mC*rArCrArUrCrArUrUrArGrArUrGrUrGrArGrGrGrCrArArUrUrUrUrArUr |
| targeting hAPOA1 | | hAPOA1 | | sequence | | GrUrCrCrUrArArUrArUrUrGrArGrGrGrUrArUrUrArGrGrUrArArCrArArCrArCrUrUr |
| | | gC3 | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrArGr |
| | | | | | | CrGrCrUrCrUrCrGrArGrGrArG*mU*mA*mC |
| DNA Sequence of | 1238 | 119-2 | Nucleotide | Artificial | | TAGTGTACTCCTCGAGAGCG |
| hAPOA1 Target | | hAPOA1 | | sequence | | |
| Site | | gA1 | | | | |
| DNA Sequence of | 1239 | 119-2 | Nucleotide | Artificial | | CGCTGAGCGTGCTCAGATGC |
| hAPOA1 Target | | hAPOA1 | | sequence | | |
| Site | | gB1 | | | | |
| DNA Sequence of | 1240 | 119-2 | Nucleotide | Artificial | | CCTTGAGAGCCTCAAGGCGC |
| hAPOAl Target | | hAPOAl | | sequence | | |
| Site | | gC1 | | | | |
| DNA Sequence of | 1241 | 119-2 | Nucleotide | Artificial | | CTTGCAGCTCGTGCAGCTTC |
| hAPOA1 Target | | hAPOA1 | | sequence | | |
| Site | | gD1 | | | | |
| DNA Sequence of | 1242 | 119-2 | Nucleotide | Artificial | | GGCGCGCGCCCTCTTGGAGC |
| hAPOA1 Target | | hAPOA1 | | sequence | | |
| Site | | gE1 | | | | |
| DNA Sequence of | 1243 | 119-2 | Nucleotide | Artificial | | GGCGGTAGAGCTCCATCTCC |
| hAPOAl Target | | hAPOA1 | | sequence | | |
| Site | | gF1 | | | | |
| DNA Sequence of | 1244 | 119-2 | Nucleotide | Artificial | | TCTGGAAGTCGTCCAGGTAG |
| hAPOA1 Target | | hAPOA1 | | sequence | | |
| Site | | gG1 | | | | |
| DNA Sequence of | 1245 | 119-2 | Nucleotide | Artificial | | GGAAGTCGTCCAGGTAGGGC |
| hAPOA1 Target | | hAPOA1 | | sequence | | |
| Site | | gH1 | | | | |
| DNA Sequence of | 1246 | 119-2 | Nucleotide | Artificial | | CCTCCTCCAGATCCTTGCTC |
| hAPOAl Target | | hAPOA1 | | sequence | | |
| Site | | gA2 | | | | |
| DNA Sequence of | 1247 | 119-2 | Nucleotide | Artificial | | CCAGGTTATCCCAGAACTCC |
| hAPOAl Target | | hAPOA1 | | sequence | | |
| Site | | gB2 | | | | |
| DNA Sequence of | 1248 | 119-2 | Nucleotide | Artificial | | CAGGTTATCCCAGAACTCCT |
| hAPOAl Target | | hAPOA1 | | sequence | | |
| Site | | gC2 | | | | |
| DNA Sequence of | 1249 | 119-2 | Nucleotide | Artificial | | AGGTTATCCCAGAACTCCTG |
| hAPOAl Target | | hAPOA1 | | sequence | | |
| Site | | gD2 | | | | |
| DNA Sequence of | 1250 | 119-2 | Nucleotide | Artificial | | CGCAGCTTGCTGAAGGTGGA |
| hAPOA1 Target | | hAPOA1 | | sequence | | |
| Site | | gE2 | | | | |
| DNA Sequence of | 1251 | 119-2 | Nucleotide | Artificial | | GTCTCACCCTCCAGCCTAAA |
| hAPOAl Target | | hAPOA1 | | sequence | | |
| Site | | gF2 | | | | |
| DNA Sequence of | 1252 | 119-2 | Nucleotide | Artificial | | GCAAGCTGCGCGAACAGCTC |
| hAPOA1 Target | | hAPOA1 | | sequence | | |
| Site | | gG2 | | | | |
| DNA Sequence of | 1253 | 119-2 | Nucleotide | Artificial | | GGGATAACCTGGAAAAGGAG |
| hAPOA1 Target | | hAPOA1 | | sequence | | |
| Site | | gH2 | | | | |
| DNA Sequence of | 1254 | 119-2 | Nucleotide | Artificial | | AGAAGAAGTGGCAGGAGGAG |
| hAPOAl Target | | hAPOA1 | | sequence | | |
| Site | | gA3 | | | | |
| DNA Sequence of | 1255 | 119-2 | Nucleotide | Artificial | | AGGTCAGCTTCCTGAGCGCT |
| hAPOAl Target | | hAPOA1 | | sequence | | |
| Site | | gB3 | | | | |
| DNA Sequence of | 1256 | 119-2 | Nucleotide | Artificial | | TGAGCGCTCTCGAGGAGTAC |
| hAPOA1 Target | | hAPOA1 | | sequence | | |
| Site | | gC3 | | | | |
| MG119-2 sgRNA | 1257 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArUrUr |
| | | | | | | UrUrGrUrUrUrUrUrCrCrArArA*mC*mU*mG |
| MG119-2 sgRNA | 1258 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrUrUr |
| | | | | | | UrUrCrCrArArArCrUrGrCrUrU*mC*mU*mC |
| MG119-2 sgRNA | 1259 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrUr |
| | | | | | | UrCrCrArArArCrUrGrCrUrUrC*mU*mC*mC |
| MG119-2 sgRNA | 1260 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrCrAr |
| | | | | | | ArArCrUrGrCrUrUrCrUrCrCrU*mC*mU*mU |
| MG119-2 sgRNA | 1261 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrArAr |
| | | | | | | ArCrUrGrCrUrUrCrUrCrCrUrC*mU*mU*mG |
| MG119-2 sgRNA | 1262 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArArAr |
| | | | | | | CrUrGrCrUrUrCrUrCrCrUrCrU*mU*mG*mG |
| MG119-2 sgRNA | 1263 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArArCr |
| | | | | | | UrGrCrUrUrCrUrCrCrUrCrUrU*mG*mG*mG |
| MG119-2 sgRNA | 1264 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH1 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrCr |
| | | | | | | UrUrGrGrGrArArGrUrGrUrArA*mG*mG*mA |
| MG119-2 sgRNA | 1265 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrGrAr |
| | | | | | | GrArGrGrGrUrArGrCrGrCrArG*mG*mG*mU |
| MG119-2 sgRNA | 1266 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrCrCr |
| | | | | | | CrArCrCrCrCrCrUrGrCrCrArA*mG*mC*mU |
| MG119-2 sgRNA | 1267 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrGrGrCrA |
| | | | | | | ArGrGrArGrArGrArGrArUrGrG*mC*mU*mC |
| MG119-2 sgRNA | 1268 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrUr |
| | | | | | | CrArCrCrArArUrCrCrUrGrUrC*mC*mC*mU |
| MG119-2 sgRNA | 1269 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE2 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrUrGrUrCr |
| | | | | | | ArCrCrArArUrCrCrUrGrUrCrC*mC*mU*mA |
| MG119-2 sgRNA | 1270 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrUrCrAr |
| | | | | | | CrCrArArUrCrCrUrGrUrCrCrC*mU*mA*mG |
| MG119-2 sgRNA | 1271 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrGrUr |
| | | | | | | UrGrUrCrCrArGrArArArArArC*mG*mG*mU |
| MG119-2 sgRNA | 1272 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH2 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrUrUr |
| | | | | | | UrCrCrUrUrCrUrCrCrUrUrCrU*mG*mG*mG |
| MG119-2 sgRNA | 1273 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrUr |
| | | | | | | CrUrCrCrUrUrCrUrGrGrGrGrC*mC*mU*mG |
| MG119-2 sgRNA | 1274 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrUrC |
| | | | | | | UrCrCrUrUrCrUrGrGrGrGrCrC*mU*mG*mU |
| MG119-2 sgRNA | 1275 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrCrUr |
| | | | | | | CrCrUrUrCrUrGrGrGrGrCrCrU*mG*mU*mG |
| MG119-2 sgRNA | 1276 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrUr |
| | | | | | | CrUrGrGrGrGrCrCrUrGrUrGrC*mC*mA*mU |
| MG119-2 sgRNA | 1277 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrGrGr |
| | | | | | | CrCrUrGrUrGrCrCrArUrCrUrC*mU*mC*mG |
| MG119-2 sgRNA | 1278 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrArGr |
| | | | | | | GrArUrGrGrCrCrUrUrCrUrCrC*mG*mA*mC |
| MG119-2 sgRNA | 1279 | 119-2 | Nucleotide | Artificial | | mU*mA*mC*rArCrArUrCrArUrUrArGrArUrGrUrGrArGrGrGrCrArArUrUrUrUrArUr |
| targeting AAVSI | | AAVS1 gG3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArGrGr |
| | | | | | | ArUrGrGrCrCrUrUrCrUrCrCrG*mA*mC*mG |
| MG119-2 sgRNA | 1280 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH3 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrGrAr |
| | | | | | | CrGrGrArUrGrUrCrUrCrCrCrU*mU*mG*mC |
| MG119-2 sgRNA | 1281 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrUrA |
| | | | | | | GrGrCrCrUrGrCrArUrCrArUrC*mA*mC*mC |
| MG119-2 sgRNA | 1282 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrUrGr |
| | | | | | | GrArCrArArCrCrCrCrArArArG*mU*mA*mC |
| MG119-2 sgRNA | 1283 | 119-2 | Nucleotide | Artificial | | mU*mA*mC*ArCrArUrCrArUrUrArGrArUrGrUrGrArGrGrGrCrArArUrUrUrUrArUr |
| targeting AAVS1 | | AAVS1 gC4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrGr |
| | | | | | | ArCrArArCrCrCrCrArArArGrU*mA*mC*mC |
| MG119-2 sgRNA | 1284 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrGrAr |
| | | | | | | CrArArCrCrCrCrArArArGrUrA*mC*mC*mC |
| MG119-2 sgRNA | 1285 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrArCr |
| | | | | | | ArArCrCrCrCrArArArGrUrArC*mC*mC*mC |
| MG119-2 sgRNA | 1286 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrCrCrAr |
| | | | | | | CrCrUrCrUrCrCrArUrCrCrUrC*mU*mU*mnG |
| MG119-2 sgRNA | 1287 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrGr |
| | | | | | | CrCrUrGrGrArCrArCrCrCrCrG*mU*mU*mC |
| MG119-2 sgRNA | 1288 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH4 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrGrC |
| | | | | | | CrUrGrGrArCrArCrCrCrCrGrU*mU*mC*mU |
| MG119-2 sgRNA | 1289 | 119-2 | Nucleotide | Artificial | | mU*mA*mC*rArCrArUrCrArUrUrArGrArUrGrUrGrArGrGrGrCrArArUrUrUrUrArU |
| targeting AAVS1 | | AAVS1 gA5 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrCrCrUrG |
| | | | | | | GrArCrArCrCrCrCrGrUrUrCrU*mC*mC*mU |
| MG119-2 sgRNA | 1290 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrGr |
| | | | | | | UrGrGrArUrUrCrGrGrGrUrCrA*mC*mC*mU |
| MG119-2 sgRNA | 1291 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrUrCr |
| | | | | | | ArCrCrUrCrUrCrArCrUrCrCrU*mU*mU*mC |
| MG119-2 sgRNA | 1292 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrUrUr |
| | | | | | | GrGrGrCrArGrCrUrCrCrCrCrU*mA*mC*mC |
| MG119-2 sgRNA | 1293 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrUrGr |
| | | | | | | GrGrCrArGrCrUrCrCrCrCrUrA*mC*mC*mC |
| MG119-2 sgRNA | 1294 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrCrAr |
| | | | | | | GrCrUrCrCrCrCrUrArCrCrCrC*mC*mC*mU |
| MG119-2 sgRNA | 1295 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrCrC |
| | | | | | | CrCrCrUrGrUrCrArUrGrGrCrA*mU*mC*mU |
| MG119-2 sgRNA | 1296 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH5 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrGrGr |
| | | | | | | GrUrCrCrGrArGrArGrCrUrCrA*mG*mC*mU |
| MG119-2 sgRNA | 1297 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrCrUr |
| | | | | | | CrCrArArCrCrCrGrGrGrCrCrC*mC*mU*mA |
| MG119-2 sgRNA | 1298 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrCrAr |
| | | | | | | ArCrCrCrGrGrGrCrCrCrCrUrA*mU*mG*mU |
| MG119-2 sgRNA | 1299 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrArCr |
| | | | | | | ArGrCrArUrGrUrUrUrGrCrUrG*mC*mC*mU |
| MG119-2 sgRNA | 1300 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrCr |
| | | | | | | CrUrCrCrArGrGrGrArUrCrCrU*mG*mU*mG |
| MG119-2 sgRNA | 1301 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArGrGr |
| | | | | | | GrCrCrGrGrUrUrArArUrGrUrG*mG*mC*mU |
| MG119-2 sgRNA | 1302 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrGrGrU |
| | | | | | | ArCrUrUrUrUrArUrCrUrGrUrC*mC*mC*mC |
| MG119-2 sgRNA | 1303 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArUrCrUr |
| | | | | | | GrUrCrCrCrCrUrCrCrArCrCrC*mC*mA*mC |
| MG119-2 sgRNA | 1304 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH6 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrUrGr |
| | | | | | | UrCrCrCrCrUrCrCrArCrCrCrC*mA*mC*mA |
| MG119-2 sgRNA | 1305 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArGrUr |
| | | | | | | CrUrCrCrUrGrArUrArUrUrGrG*mG*mU*mC |
| MG119-2 sgRNA | 1306 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrArUr |
| | | | | | | CrUrGrGrUrGrArCrArCrArCrC*mC*mC*mC |
| MG119-2 sgRNA | 1307 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrGr |
| | | | | | | ArGrCrCrArUrCrUrCrUrCrUrC*mC*mU*mU |
| MG119-2 sgRNA | 1308 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrGrGrAr |
| | | | | | | GrCrCrArUrCrUrCrUrCrUrCrC*mU*mU*mG |
| MG119-2 sgRNA | 1309 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrUrA |
| | | | | | | CrGrArUrGrGrArGrCrCrArGrA*mG*mA*mG |
| MG119-2 sgRNA | 1310 | 119-2 | Nucleotide | Artificial | | mU*mA*mC*rArCrArUrCrArUrUrArGrArUrGrUrGrArGrGrGrCrArArUrUrUrUrArU |
| targeting AAVS1 | | AAVS1 gF7 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrCrArGrU |
| | | | | | | GrGrCrCrArCrCrCrUrGrCrGrC*mU*mA*mC |
| MG119-2 sgRNA | 1311 | 119-2 | Nucleotide | Artificial | | mU*mA*mC*rArCrArUrCrArUrUrArGrArUrGrUrGrArGrGrGrCrArArUrUrUrUrArU |
| targeting AAVS1 | | AAVS1 gG7 | | sequence | | GrUrCrCrUrArArUrArUrUrGrArGrGrGrUrArUrUrArGrGrUrArArCrArArCrArCrUrU |
| | | | | | | UrGrUrUrUrUrArArUrArGrArArCrArGrUrGrUrCrUrArArUrArArCrCrArCrUrCrGrA |
| | | | | | | UrArArGrArCrCrArUrArUrGrArArArGrUrArUrGrGrUrUrArUrArArArArCrArCrUrGr |
| | | | | | | ArUrCrCrUrGrGrUrGrCrUrGrC*mA*mG*mC |
| MG119-2 sgRNA | 1312 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH7 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrUrGrAr |
| | | | | | | UrCrCrUrGrGrUrGrCrUrGrCrA*mG*mC*mU |
| MG119-2 sgRNA | 1313 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrUrArCr |
| | | | | | | ArCrUrUrCrCrCrArArGrArGrG*mA*mG*mA |
| MG119-2 sgRNA | 1314 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrArArGr |
| | | | | | | ArGrGrArGrArArGrCrArGrUrU*mU*mG*mG |
| MG119-2 sgRNA | 1315 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrArArAr |
| | | | | | | ArArCrArArArArUrCrArGrArA*mU*mA*mA |
| MG119-2 sgRNA | 1316 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArArCrU |
| | | | | | | UrUrGrGrCrUrCrUrUrCrArCrC*mU*mU*mU |
| MG119-2 sgRNA | 1317 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gE8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrGrCrUrCr |
| | | | | | | UrUrCrArCrCrUrUrUrCrUrArG*mU*mC*mC |
| MG119-2 sgRNA | 1318 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gF8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrUr |
| | | | | | | UrCrUrArGrUrCrCrCrCrArArU*mU*mU*mA |
| MG119-2 sgRNA | 1319 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gG8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArGrUr |
| | | | | | | CrCrCrCrArArUrUrUrArUrArU*mU*mG*mU |
| MG119-2 sgRNA | 1320 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gH8 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArGrUrCr |
| | | | | | | CrCrCrArArUrUrUrArUrArUrU*mG*mU*mU |
| MG119-2 sgRNA | 1321 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gA9 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrArUrUr |
| | | | | | | GrUrUrCrCrUrCrCrGrUrGrCrG*mU*mC*mA |
| MG119-2 sgRNA | 1322 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gB9 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrUrCrCrGr |
| | | | | | | UrGrCrGrUrCrArGrUrUrUrUrA*mC*mC*mU |
| MG119-2 sgRNA | 1323 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gC9 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrArCrCrU |
| | | | | | | GrUrGrArGrArUrArArGrGrCrC*mA*mG*mU |
| MG119-2 sgRNA | 1324 | 119-2 | Nucleotide | Artificial | | mU*mA*mC*rArCrArUrCrArUrUrArGrArUrGrUrGrArGrGrGrCrArArUrUrUrUrArUr |
| targeting AAVS1 | | AAVS1 gD9 | | sequence | | GrUrCrCrUrArArUrArUrUrGrArGrGrGrUrArUrUrArGrGrUrArArCrArArCrArCrUrUr |
| | | | | | | UrGrUrUrUrUrArArUrArGrArArCrArGrUrGrUrCrUrArArUrArArCrCrArCrUrCrGrAr |
| | | | | | | UrArArGrArCrCrArUrArUrGrArArArGrUrArUrGrGrUrUrArUrArArArArCrCrCrUrGr |
| | | | | | | UrGrArGrArUrArArGrGrCrCrA*mG*mU*mA |
| DNA Sequence of | 1325 | 119-2 | Nucleotide | Artificial | | GATTTTGTTTTTCCAAACTG |
| AAVS1 Target Site | | AAVS1 gAl | | sequence | | |
| DNA Sequence of | 1326 | 119-2 | Nucleotide | Artificial | | GTTTTTCCAAACTGCTTCTC |
| AAVS1 Target Site | | AAVS1 gB1 | | sequence | | |
| DNA Sequence of | 1327 | 119-2 | Nucleotide | Artificial | | TTTTTCCAAACTGCTTCTCC |
| AAVS1 Target Site | | AAVS1 gCl | | sequence | | |
| DNA Sequence of | 1328 | 119-2 | Nucleotide | Artificial | | TCCAAACTGCTTCTCCTCTT |
| AAVS1 Target Site | | AAVS1 gD1 | | sequence | | |
| DNA Sequence of | 1329 | 119-2 | Nucleotide | Artificial | | CCAAACTGCTTCTCCTCTTG |
| AAVS1 Target Site | | AAVS1 gE1 | | sequence | | |
| DNA Sequence of | 1330 | 119-2 | Nucleotide | Artificial | | CAAACTGCTTCTCCTCTTGG |
| AAVS1 Target Site | | AAVS1 gF1 | | sequence | | |
| DNA Sequence of | 1331 | 119-2 | Nucleotide | Artificial | | AAACTGCTTCTCCTCTTGGG |
| AAVS1 Target Site | | AAVS1 gG1 | | sequence | | |
| DNA Sequence of | 1332 | 119-2 | Nucleotide | Artificial | | CCTCTTGGGAAGTGTAAGGA |
| AAVS1 Target Site | | AAVS1 gH1 | | sequence | | |
| DNA Sequence of | 1333 | 119-2 | Nucleotide | Artificial | | GGGAGAGGGTAGCGCAGGGT |
| AAVS1 Target Site | | AAVS1 gA2 | | sequence | | |
| DNA Sequence of | 1334 | 119-2 | Nucleotide | Artificial | | CTCCCACCCCCTGCCAAGCT |
| AAVS1 Target Site | | AAVS1 gB2 | | sequence | | |
| DNA Sequence of | 1335 | 119-2 | Nucleotide | Artificial | | GGCAAGGAGAGAGATGGCTC |
| AAVS1 Target Site | | AAVS1 gC2 | | sequence | | |
| DNA Sequence of | 1336 | 119-2 | Nucleotide | Artificial | | CTGTCACCAATCCTGTCCCT |
| AAVS1 Target Site | | AAVS1 gD2 | | sequence | | |
| DNA Sequence of | 1337 | 119-2 | Nucleotide | Artificial | | TGTCACCAATCCTGTCCCTA |
| AAVS1 Target Site | | AAVS1 gE2 | | sequence | | |
| DNA Sequence of | 1338 | 119-2 | Nucleotide | Artificial | | GTCACCAATCCTGTCCCTAG |
| AAVS1 Target Site | | AAVS1 gF2 | | sequence | | |
| DNA Sequence of | 1339 | 119-2 | Nucleotide | Artificial | | GGGTTGTCCAGAAAAACGGT |
| AAVS1 Target Site | | AAVS1 gG2 | | sequence | | |
| DNA Sequence of | 1340 | 119-2 | Nucleotide | Artificial | | CTTTTCCTTCTCCTTCTGGG |
| AAVS1 Target Site | | AAVS1 gH2 | | sequence | | |
| DNA Sequence of | 1341 | 119-2 | Nucleotide | Artificial | | CCTTCTCCTTCTGGGGCCTG |
| AAVS1 Target Site | | AAVS1 gA3 | | sequence | | |
| DNA Sequence of | 1342 | 119-2 | Nucleotide | Artificial | | CTTCTCCTTCTGGGGCCTGT |
| AAVS1 Target Site | | AAVS1 gB3 | | sequence | | |
| DNA Sequence of | 1343 | 119-2 | Nucleotide | Artificial | | TTCTCCTTCTGGGGCCTGTG |
| AAVS1 Target Site | | AAVS1 gC3 | | sequence | | |
| DNA Sequence of | 1344 | 119-2 | Nucleotide | Artificial | | CCTTCTGGGGCCTGTGCCAT |
| AAVS1 Target Site | | AAVS1 gD3 | | sequence | | |
| DNA Sequence of | 1345 | 119-2 | Nucleotide | Artificial | | GGGGCCTGTGCCATCTCTCG |
| AAVS1 Target Site | | AAVS1 gE3 | | sequence | | |
| DNA Sequence of | 1346 | 119-2 | Nucleotide | Artificial | | TTAGGATGGCCTTCTCCGAC |
| AAVS1 Target Site | | AAVS1 gF3 | | sequence | | |
| DNA Sequence of | 1347 | 119-2 | Nucleotide | Artificial | | TAGGATGGCCTTCTCCGACG |
| AAVS1 Target Site | | AAVS1 gG3 | | sequence | | |
| DNA Sequence of | 1348 | 119-2 | Nucleotide | Artificial | | CCGACGGATGTCTCCCTTGC |
| AAVS1 Target Site | | AAVS1 gH3 | | sequence | | |
| DNA Sequence of | 1349 | 119-2 | Nucleotide | Artificial | | TGTAGGCCTGCATCATCACC |
| AAVS1 Target Site | | AAVS1 gA4 | | sequence | | |
| DNA Sequence of | 1350 | 119-2 | Nucleotide | Artificial | | TCTGGACAACCCCAAAGTAC |
| AAVS1 Target Site | | AAVS1 gB4 | | sequence | | |
| DNA Sequence of | 1351 | 119-2 | Nucleotide | Artificial | | CTGGACAACCCCAAAGTACC |
| AAVS1 Target Site | | AAVS1 gC4 | | sequence | | |
| DNA Sequence of | 1352 | 119-2 | Nucleotide | Artificial | | TGGACAACCCCAAAGTACCC |
| AAVS1 Target Site | | AAVS1 gD4 | | sequence | | |
| DNA Sequence of | 1353 | 119-2 | Nucleotide | Artificial | | GGACAACCCCAAAGTACCCC |
| AAVS1 Target Site | | AAVS1 gE4 | | sequence | | |
| DNA Sequence of | 1354 | 119-2 | Nucleotide | Artificial | | GCCACCTCTCCATCCTCTTG |
| AAVS1 Target Site | | AAVS1 gF4 | | sequence | | |
| DNA Sequence of | 1355 | 119-2 | Nucleotide | Artificial | | TTTGCCTGGACACCCCGTTC |
| AAVS1 Target Site | | AAVS1 gG4 | | sequence | | |
| DNA Sequence of | 1356 | 119-2 | Nucleotide | Artificial | | TTGCCTGGACACCCCGTTCT |
| AAVS1 Target Site | | AAVS1 gH4 | | sequence | | |
| DNA Sequence of | 1357 | 119-2 | Nucleotide | Artificial | | CCTGGACACCCCGTTCTCCT |
| AAVS1 Target Site | | AAVS1 gA5 | | sequence | | |
| DNA Sequence of | 1358 | 119-2 | Nucleotide | Artificial | | CCTGTGGATTCGGGTCACCT |
| AAVS1 Target Site | | AAVS1 gB5 | | sequence | | |
| DNA Sequence of | 1359 | 119-2 | Nucleotide | Artificial | | GGTCACCTCTCACTCCTTTC |
| AAVS1 Target Site | | AAVS1 gC5 | | sequence | | |
| DNA Sequence of | 1360 | 119-2 | Nucleotide | Artificial | | ATTTGGGCAGCTCCCCTACC |
| AAVS1 Target Site | | AAVS1 gD5 | | sequence | | |
| DNA Sequence of | 1361 | 119-2 | Nucleotide | Artificial | | TTTGGGCAGCTCCCCTACCC |
| AAVS1 Target Site | | AAVS1 gE5 | | sequence | | |
| DNA Sequence of | 1362 | 119-2 | Nucleotide | Artificial | | GGCAGCTCCCCTACCCCCCT |
| AAVS1 Target Site | | AAVS1 gF5 | | sequence | | |
| DNA Sequence of | 1363 | 119-2 | Nucleotide | Artificial | | AGCCCCCTGTCATGGCATCT |
| AAVS1 Target Site | | AAVS1 gG5 | | sequence | | |
| DNA Sequence of | 1364 | 119-2 | Nucleotide | Artificial | | AGGGGTCCGAGAGCTCAGCT |
| AAVS1 Target Site | | AAVS1 gH5 | | sequence | | |
| DNA Sequence of | 1365 | 119-2 | Nucleotide | Artificial | | TCCTCCAACCCGGGCCCCTA |
| AAVS1 Target Site | | AAVS1 gA6 | | sequence | | |
| DNA Sequence of | 1366 | 119-2 | Nucleotide | Artificial | | TCCAACCCGGGCCCCTATGT |
| AAVS1 Target Site | | AAVS1 gB6 | | sequence | | |
| DNA Sequence of | 1367 | 119-2 | Nucleotide | Artificial | | GGACAGCATGTTTGCTGCCT |
| AAVS1 Target Site | | AAVS1 gC6 | | sequence | | |
| DNA Sequence of | 1368 | 119-2 | Nucleotide | Artificial | | CTGCCTCCAGGGATCCTGTG |
| AAVS1 Target Site | | AAVS1 gD6 | | sequence | | |
| DNA Sequence of | 1369 | 119-2 | Nucleotide | Artificial | | CAGGGCCGGTTAATGTGGCT |
| AAVS1 Target Site | | AAVS1 gE6 | | sequence | | |
| DNA Sequence of | 1370 | 119-2 | Nucleotide | Artificial | | GGGTACTTTTATCTGTCCCC |
| AAVS1 Target Site | | AAVS1 gF6 | | sequence | | |
| DNA Sequence of | 1371 | 119-2 | Nucleotide | Artificial | | ATCTGTCCCCTCCACCCCAC |
| AAVS1 Target Site | | AAVS1 gG6 | | sequence | | |
| DNA Sequence of | 1372 | 119-2 | Nucleotide | Artificial | | TCTGTCCCCTCCACCCCACA |
| AAVS1 Target Site | | AAVS1 gH6 | | sequence | | |
| DNA Sequence of | 1373 | 119-2 | Nucleotide | Artificial | | TAGTCTCCTGATATTGGGTC |
| AAVS1 Target Site | | AAVS1 gA7 | | sequence | | |
| DNA Sequence of | 1374 | 119-2 | Nucleotide | Artificial | | TTATCTGGTGACACACCCCC |
| AAVS1 Target Site | | AAVS1 gB7 | | sequence | | |
| DNA Sequence of | 1375 | 119-2 | Nucleotide | Artificial | | CTGGAGCCATCTCTCTCCTT |
| AAVS1 Target Site | | AAVS1 gC7 | | sequence | | |
| DNA Sequence of | 1376 | 119-2 | Nucleotide | Artificial | | TGGAGCCATCTCTCTCCTTG |
| AAVS1 Target Site | | AAVS1 gD7 | | sequence | | |
| DNA Sequence of | 1377 | 119-2 | Nucleotide | Artificial | | CTTACGATGGAGCCAGAGAG |
| AAVS1 Target Site | | AAVS1 gE7 | | sequence | | |
| DNA Sequence of | 1378 | 119-2 | Nucleotide | Artificial | | CAGTGGCCACCCTGCGCTAC |
| AAVS1 Target Site | | AAVS1 gF7 | | sequence | | |
| DNA Sequence of | 1379 | 119-2 | Nucleotide | Artificial | | ACTGATCCTGGTGCTGCAGC |
| AAVS1 Target Site | | AAVS1 gG7 | | sequence | | |
| DNA Sequence of | 1380 | 119-2 | Nucleotide | Artificial | | CTGATCCTGGTGCTGCAGCT |
| AAVS1 Target Site | | AAVS1 gH7 | | sequence | | |
| DNA Sequence of | 1381 | 119-2 | Nucleotide | Artificial | | TTACACTTCCCAAGAGGAGA |
| AAVS1 Target Site | | AAVS1 gA8 | | sequence | | |
| DNA Sequence of | 1382 | 119-2 | Nucleotide | Artificial | | CAAGAGGAGAAGCAGTTTGG |
| AAVS1 Target Site | | AAVS1 gB8 | | sequence | | |
| DNA Sequence of | 1383 | 119-2 | Nucleotide | Artificial | | GAAAAACAAAATCAGAATAA |
| AAVS1 Target Site | | AAVS1 gC8 | | sequence | | |
| DNA Sequence of | 1384 | 119-2 | Nucleotide | Artificial | | AACTTTGGCTCTTCACCTTT |
| AAVS1 Target Site | | AAVS1 gD8 | | sequence | | |
| DNA Sequence of | 1385 | 119-2 | Nucleotide | Artificial | | GCTCTTCACCTTTCTAGTCC |
| AAVS1 Target Site | | AAVS1 gE8 | | sequence | | |
| DNA Sequence of | 1386 | 119-2 | Nucleotide | Artificial | | CCTTTCTAGTCCCCAATTTA |
| AAVS1 Target Site | | AAVS1 gF8 | | sequence | | |
| DNA Sequence of | 1387 | 119-2 | Nucleotide | Artificial | | TAGTCCCCAATTTATATTGT |
| AAVS1 Target Site | | AAVS1 gG8 | | sequence | | |
| DNA Sequence of | 1388 | 119-2 | Nucleotide | Artificial | | AGTCCCCAATTTATATTGTT |
| AAVS1 Target Site | | AAVS1 gH8 | | sequence | | |
| DNA Sequence of | 1389 | 119-2 | Nucleotide | Artificial | | TATTGTTCCTCCGTGCGTCA |
| AAVS1 Target Site | | AAVS1 gA9 | | sequence | | |
| DNA Sequence of | 1390 | 119-2 | Nucleotide | Artificial | | TCCGTGCGTCAGTTTTACCT |
| AAVS1 Target Site | | AAVS1 gB9 | | sequence | | |
| DNA Sequence of | 1391 | 119-2 | Nucleotide | Artificial | | ACCTGTGAGATAAGGCCAGT |
| AAVS1 Target Site | | AAVS1 gC9 | | sequence | | |
| DNA Sequence of | 1392 | 119-2 | Nucleotide | Artificial | | CCTGTGAGATAAGGCCAGTA |
| AAVS1 Target Site | | AAVS1 gD9 | | sequence | | |
| MG119-28 sgRNA | 1393 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrCrArArCrUrUrUrUrArUrUrCrUr |
| | | | | | | ArU*mU*mU*mU |
| MG119-28 sgRNA | 1394 | 119-28 | Nucleotide | Artificial | | mU*U*mG*rArArArUrArArArArUrGrArArUrUrUrCrArArArCrCrCrCrUrUrCrGrGr |
| targeting hALB | | hALB gB1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrCrUrArUrUrUrUrCrCrCrArGrUr |
| | | | | | | ArA*mA*mA*mU |
| MG119-28 sgRNA | 1395 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrUrArArArCrUrCrUrGrCrArUrCrUr |
| | | | | | | UrU*mA*mA*mA |
| MG119-28 sgRNA | 1396 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArGrArArUrUrArUrUrUrUrGrGrCr |
| | | | | | | ArU*mU*mU*mA |
| MG119-28 sgRNA | 1397 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrUrGrGrCrArUrUrUrArUrUrUr |
| | | | | | | CrU*mA*mA*mA |
| MG119-28 sgRNA | 1398 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gF1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrArUrUrUrArUrUrUrCrUrArArAr |
| | | | | | | ArU*mG*mG*mC |
| MG119-28 sgRNA | 1399 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrCrUrArArArArUrGrGrCrArUr |
| | | | | | | ArG*mU*mA*mU |
| MG119-28 sgRNA | 1400 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gH1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArUrUrUrGrUrGrArArGrUrCrUrUr |
| | | | | | | ArC*mA*mA*mG |
| MG119-28 sgRNA | 1401 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrGrArArGrUrCrUrUrArCrArArGrGr |
| | | | | | | UrU*mA*mU*mC |
| MG119-28 sgRNA | 1402 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gB2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrArArGrGrUrUrArUrCrUrUrArUrUr |
| | | | | | | ArA*mU*mA*mA |
| MG119-28 sgRNA | 1403 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrUrArUrUrArArUrArArArArUr |
| | | | | | | UrC*mA*mA*mA |
| MG119-28 sgRNA | 1404 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrArArUrArArArArUrUrCrArArAr |
| | | | | | | CrA*mU*mC*mC |
| MG119-28 sgRNA | 1405 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArUrArArArArUrUrCrArArArCrArUr |
| | | | | | | CrC*mU*mA*mG |
| MG119-28 sgRNA | 1406 | 119-28 | Nucleotide | Artificial | | mUmUmG*rArArArUrArArArArUrGrArArUrUrUrCrArArArCrCrCrCrUrUrCrGrGr |
| targeting hALB | | hALB gF2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrArGrUrCrArCrUrArArArCrArArUr |
| | | | | | | UrC*mU*mG*mA |
| MG119-28 sgRNA | 1407 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrUrArGrGrArUrGrUrUrUrGrArAr |
| | | | | | | UrU*mU*mU*mA |
| MG119-28 sgRNA | 1408 | 119-28 | Nucleotide | Artificial | | mU*U*mG*rArArArUrArArArArUrGrArArUrUrUrCrArArArCrCrCrCrUrUrCrGrGr |
| targeting hALB | | hALB gH2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArUrUrUrUrArUrUrArArUrArArGr |
| | | | | | | ArU*mA*mA*mC |
| MG119-28 sgRNA | 1409 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrArArUrArArGrArUrArArCrCrUr |
| | | | | | | UrG*mU*mA*mA |
| MG119-28 sgRNA | 1410 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gB3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArUrArArGrArUrArArCrCrUrUrGrUr |
| | | | | | | ArA*mG*mA*mC |
| MG119-28 sgRNA | 1411 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArArGrArCrUrUrCrArCrArArArUr |
| | | | | | | ArC*mA*mA*mA |
| MG119-28 sgRNA | 1412 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArArArUrArArArUrGrCrCrArArAr |
| | | | | | | ArU*mA*mA*mU |
| MG119-28 sgRNA | 1413 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArGrArUrGrCrArGrArGrUrUrUrAr |
| | | | | | | CrU*mA*mA*mA |
| MG119-28 sgRNA | 1414 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gF3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrArArArArCrUrUrUrArUrUrUrUr |
| | | | | | | ArC*mU*mG*mG |
| MG119-28 sgRNA | 1415 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrUrArCrUrGrGrGrArArArArUr |
| | | | | | | ArG*mA*mA*mU |
| MG119-28 sgRNA | 1416 | 119-28 | Nucleotide | Artificial | | mU*mU*G*rArArArUrArArArArUrGrArArUrUrUrCrArArArCrCrCrCrUrUrCrGrGr |
| targeting hALB | | hALB gH3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrGrGrGrArArArArUrArGrArArUr |
| | | | | | | ArA*mA*mA*mG |
| MG119-28 sgRNA | 1417 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArCrArArUrArGrArArArArArUrGr |
| | | | | | | GrA*mU*mU*mU |
| MG119-28 sgRNA | 1418 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gB4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArUrArArUrUrUrUrUrArArArArUrAr |
| | | | | | | GrU*mA*mU*mU |
| MG119-28 sgRNA | 1419 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArArUrArGrUrArUrUrCrUrUrGrGr |
| | | | | | | UrA*mA*mU*mU |
| MG119-28 sgRNA | 1420 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrUrArArUrUrGrArArUrUrArUrUrCr |
| | | | | | | UrU*mC*mU*mG |
| MG119-28 sgRNA | 1421 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArUrUrArUrUrCrUrUrCrUrGrUrUr |
| | | | | | | UrA*mA*mA*mG |
| MG119-28 sgRNA | 1422 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gF4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrCrUrUrCrUrGrUrUrUrArArArGr |
| | | | | | | GrC*mA*mG*mA |
| MG119-28 sgRNA | 1423 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArGrGrCrArGrArArGrArArArUrAr |
| | | | | | | ArU*mU*mG*mA |
| MG119-28 sgRNA | 1424 | 119-28 | Nucleotide | Artificial | | mU*mU*mG*ArArArUrArArArArUrGrArArUrUrUrCrArArArCrCrCrCrUrUrCrGrGr |
| targeting hALB | | hALB gH4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArCrArUrCrArUrCrCrUrGrArGrUr |
| | | | | | | UrU*mU*mU*mC |
| MG119-28 sgRNA | 1425 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArArCrArArArUrGrCrArUrArArUr |
| | | | | | | CrU*mA*mA*mG |
| MG119-28 sgRNA | 1426 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gB5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrArCrUrUrCrUrUrGrUrUrUrUrCr |
| | | | | | | UrU*mC*mA*mG |
| MG119-28 sgRNA | 1427 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrUrCrUrUrGrUrUrUrUrCrUrUrCr |
| | | | | | | ArG*mU*mA*mU |
| MG119-28 sgRNA | 1428 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrUrCrUrUrCrArGrUrArUrUrUr |
| | | | | | | ArA*mC*mA*mA |
| MG119-28 sgRNA | 1429 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArCrArArUrCrCrUrUrUrUrUrUrUrUr |
| | | | | | | CrU*mU*mC*mC |
| MG119-28 sgRNA | 1430 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gF5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrGrGrGrCrArArGrGrGrArArGr |
| | | | | | | ArA*mA*mA*mA |
| MG119-28 sgRNA | 1431 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrArArArUrArCrUrGrArArGrArAr |
| | | | | | | ArA*mC*mA*mA |
| MG119-28 sgRNA | 1432 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gH5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArUrArCrUrGrArArGrArArArArCr |
| | | | | | | ArA*mG*mA*mA |
| MG119-28 sgRNA | 1433 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrUrUrUrUrArUrArUrUrUrCrUrUr |
| | | | | | | UrC*mC*mA*mU |
| MG119-28 sgRNA | 1434 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gB6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArUrUrUrCrUrUrUrCrCrArUrUrUr |
| | | | | | | GrA*mC*mU*mU |
| MG119-28 sgRNA | 1435 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gC6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArCrUrUrArGrArUrUrArUrGrCrArUr |
| | | | | | | UrU*mG*mU*mU |
| MG119-28 sgRNA | 1436 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gD6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArUrUrArUrGrCrArUrUrUrGrUrUr |
| | | | | | | UrC*mA*mA*mA |
| MG119-28 sgRNA | 1437 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gE6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrGrCrArUrUrUrGrUrUrUrCrArArAr |
| | | | | | | ArU*mA*mU*mU |
| MG119-28 sgRNA | 1438 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gF6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrCrArArArArUrArUrUrGrGrGr |
| | | | | | | CrU*mC*mU*mG |
| MG119-28 sgRNA | 1439 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gG6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrCrUrCrUrGrArUrUrCrCrUrArCr |
| | | | | | | ArG*mA*mA*mA |
| MG119-28 sgRNA | 1440 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gH6 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrCrUrUrCrUrGrCrCrUrUrUrAr |
| | | | | | | ArA*mC*mA*mG |
| MG119-28 sgRNA | 1441 | 119-28 | Nucleotide | Artificial | | mU*mU*mG*rArArArUrArArArArUrGrArArUrUrUrCrArArArCrCrCrCrUrUrCrGrGr |
| targeting hALB | | hALB gA7 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArCrArGrArArGrArArUrArArUrUr |
| | | | | | | CrA*mA*mU*mU |
| DNA Sequence of | 1442 | 119-28 | Nucleotide | Artificial | | TTCAACTTTTATTCTATTTT |
| hALB Target Site | | hALB gA1 | | sequence | | |
| DNA Sequence of | 1443 | 119-28 | Nucleotide | Artificial | | TTCTATTTTCCCAGTAAAAT |
| hALB Target Site | | hALB gB1 | | sequence | | |
| DNA Sequence of | 1444 | 119-28 | Nucleotide | Artificial | | GTAAACTCTGCATCTTTAAA |
| hALB Target Site | | hALB gC1 | | sequence | | |
| DNA Sequence of | 1445 | 119-28 | Nucleotide | Artificial | | AAGAATTATTTTGGCATTTA |
| hALB Target Site | | hALB gD1 | | sequence | | |
| DNA Sequence of | 1446 | 119-28 | Nucleotide | Artificial | | TTTTGGCATTTATTTCTAAA |
| hALB Target Site | | hALB gE1 | | sequence | | |
| DNA Sequence of | 1447 | 119-28 | Nucleotide | Artificial | | GCATTTATTTCTAAAATGGC |
| hALB Target Site | | hALB gF1 | | sequence | | |
| DNA Sequence of | 1448 | 119-28 | Nucleotide | Artificial | | TTTCTAAAATGGCATAGTAT |
| hALB Target Site | | hALB gG1 | | sequence | | |
| DNA Sequence of | 1449 | 119-28 | Nucleotide | Artificial | | TATTTGTGAAGTCTTACAAG |
| hALB Target Site | | hALB gH1 | | sequence | | |
| DNA Sequence of | 1450 | 119-28 | Nucleotide | Artificial | | TGAAGTCTTACAAGGTTATC |
| hALB Target Site | | hALB gA2 | | sequence | | |
| DNA Sequence of | 1451 | 119-28 | Nucleotide | Artificial | | CAAGGTTATCTTATTAATAA |
| hALB Target Site | | hALB gB2 | | sequence | | |
| DNA Sequence of | 1452 | 119-28 | Nucleotide | Artificial | | TCTTATTAATAAAATTCAAA |
| hALB Target Site | | hALB gC2 | | sequence | | |
| DNA Sequence of | 1453 | 119-28 | Nucleotide | Artificial | | TTAATAAAATTCAAACATCC |
| hALB Target Site | | hALB gD2 | | sequence | | |
| DNA Sequence of | 1454 | 119-28 | Nucleotide | Artificial | | ATAAAATTCAAACATCCTAG |
| hALB Target Site | | hALB gE2 | | sequence | | |
| DNA Sequence of | 1455 | 119-28 | Nucleotide | Artificial | | CAGTCACTAAACAATTCTGA |
| hALB Target Site | | hALB gF2 | | sequence | | |
| DNA Sequence of | 1456 | 119-28 | Nucleotide | Artificial | | CCTAGGATGTTTGAATTTTA |
| hALB Target Site | | hALB gG2 | | sequence | | |
| DNA Sequence of | 1457 | 119-28 | Nucleotide | Artificial | | AATTTTATTAATAAGATAAC |
| hALB Target Site | | hALB gH2 | | sequence | | |
| DNA Sequence of | 1458 | 119-28 | Nucleotide | Artificial | | TTAATAAGATAACCTTGTAA |
| hALB Target Site | | hALB gA3 | | sequence | | |
| DNA Sequence of | 1459 | 119-28 | Nucleotide | Artificial | | ATAAGATAACCTTGTAAGAC |
| hALB Target Site | | hALB gB3 | | sequence | | |
| DNA Sequence of | 1460 | 119-28 | Nucleotide | Artificial | | TAAGACTTCACAAATACAAA |
| hALB Target Site | | hALB gC3 | | sequence | | |
| DNA Sequence of | 1461 | 119-28 | Nucleotide | Artificial | | GAAATAAATGCCAAAATAAT |
| hALB Target Site | | hALB gD3 | | sequence | | |
| DNA Sequence of | 1462 | 119-28 | Nucleotide | Artificial | | AAGATGCAGAGTTTACTAAA |
| hALB Target Site | | hALB gE3 | | sequence | | |
| DNA Sequence of | 1463 | 119-28 | Nucleotide | Artificial | | CTAAAACTTTATTTTACTGG |
| hALB Target Site | | hALB gF3 | | sequence | | |
| DNA Sequence of | 1464 | 119-28 | Nucleotide | Artificial | | TTTTACTGGGAAAATAGAAT |
| hALB Target Site | | hALB gG3 | | sequence | | |
| DNA Sequence of | 1465 | 119-28 | Nucleotide | Artificial | | CTGGGAAAATAGAATAAAAG |
| hALB Target Site | | hALB gH3 | | sequence | | |
| DNA Sequence of | 1466 | 119-28 | Nucleotide | Artificial | | AACAATAGAAAAATGGATTT |
| hALB Target Site | | hALB gA4 | | sequence | | |
| DNA Sequence of | 1467 | 119-28 | Nucleotide | Artificial | | ATAATTTTTAAAATAGTATT |
| hALB Target Site | | hALB gB4 | | sequence | | |
| DNA Sequence of | 1468 | 119-28 | Nucleotide | Artificial | | AAATAGTATTCTTGGTAATT |
| hALB Target Site | | hALB gC4 | | sequence | | |
| DNA Sequence of | 1469 | 119-28 | Nucleotide | Artificial | | GTAATTGAATTATTCTTCTG |
| hALB Target Site | | hALB gD4 | | sequence | | |
| DNA Sequence of | 1470 | 119-28 | Nucleotide | Artificial | | AATTATTCTTCTGTTTAAAG |
| hALB Target Site | | hALB gE4 | | sequence | | |
| DNA Sequence of | 1471 | 119-28 | Nucleotide | Artificial | | TTCTTCTGTTTAAAGGCAGA |
| hALB Target Site | | hALB gF4 | | sequence | | |
| DNA Sequence of | 1472 | 119-28 | Nucleotide | Artificial | | AAGGCAGAAGAAATAATTGA |
| hALB Target Site | | hALB gG4 | | sequence | | |
| DNA Sequence of | 1473 | 119-28 | Nucleotide | Artificial | | AACATCATCCTGAGTTTTTC |
| hALB Target Site | | hALB gH4 | | sequence | | |
| DNA Sequence of | 1474 | 119-28 | Nucleotide | Artificial | | AAACAAATGCATAATCTAAG |
| hALB Target Site | | hALB gA5 | | sequence | | |
| DNA Sequence of | 1475 | 119-28 | Nucleotide | Artificial | | TTACTTCTTGTTTTCTTCAG |
| hALB Target Site | | hALB gB5 | | sequence | | |
| DNA Sequence of | 1476 | 119-28 | Nucleotide | Artificial | | CTTCTTGTTTTCTTCAGTAT |
| hALB Target Site | | hALB gC5 | | sequence | | |
| DNA Sequence of | 1477 | 119-28 | Nucleotide | Artificial | | TTTTCTTCAGTATTTAACAA |
| hALB Target Site | | hALB gD5 | | sequence | | |
| DNA Sequence of | 1478 | 119-28 | Nucleotide | Artificial | | ACAATCCTTTTTTTTCTTCC |
| hALB Target Site | | hALB gE5 | | sequence | | |
| DNA Sequence of | 1479 | 119-28 | Nucleotide | Artificial | | TCTGGGCAAGGGAAGAAAAA |
| hALB Target Site | | hALB gF5 | | sequence | | |
| DNA Sequence of | 1480 | 119-28 | Nucleotide | Artificial | | TTAAATACTGAAGAAAACAA |
| hALB Target Site | | hALB gG5 | | sequence | | |
| DNA Sequence of | 1481 | 119-28 | Nucleotide | Artificial | | AATACTGAAGAAAACAAGAA |
| hALB Target Site | | hALB gH5 | | sequence | | |
| DNA Sequence of | 1482 | 119-28 | Nucleotide | Artificial | | CTTTTTATATTTCTTTCCAT |
| hALB Target Site | | hALB gA6 | | sequence | | |
| DNA Sequence of | 1483 | 119-28 | Nucleotide | Artificial | | TATTTCTTTCCATTTGACTT |
| hALB Target Site | | hALB gB6 | | sequence | | |
| DNA Sequence of | 1484 | 119-28 | Nucleotide | Artificial | | ACTTAGATTATGCATTTGTT |
| hALB Target Site | | hALB gC6 | | sequence | | |
| DNA Sequence of | 1485 | 119-28 | Nucleotide | Artificial | | GATTATGCATTTGTTTCAAA |
| hALB Target Site | | hALB gD6 | | sequence | | |
| DNA Sequence of | 1486 | 119-28 | Nucleotide | Artificial | | TGCATTTGTTTCAAAATATT |
| hALB Target Site | | hALB gE6 | | sequence | | |
| DNA Sequence of | 1487 | 119-28 | Nucleotide | Artificial | | TTTCAAAATATTGGGCTCTG |
| hALB Target Site | | hALB gF6 | | sequence | | |
| DNA Sequence of | 1488 | 119-28 | Nucleotide | Artificial | | GGCTCTGATTCCTACAGAAA |
| hALB Target Site | | hALB gG6 | | sequence | | |
| DNA Sequence of | 1489 | 119-28 | Nucleotide | Artificial | | TTTCTTCTGCCTTTAAACAG |
| hALB Target Site | | hALB gH6 | | sequence | | |
| DNA Sequence of | 1490 | 119-28 | Nucleotide | Artificial | | AACAGAAGAATAATTCAATT |
| hALB Target Site | | hALB gA7 | | sequence | | |
| MG119-28 sgRNA | 1491 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gA1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArGrCrUrUrCrUrUrArGrUrGrUrArCr |
| | | | | | | UrC*mC*mU*mC |
| MG119-28 sgRNA | 1492 | 119-28 | Nucleotide | Artificial | | mUmU*mG*ArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gB1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrUrGrUrArCrUrCrCrUrCrGrArGrAr |
| | | | | | | GrC*mG*mC*mU |
| MG119-28 sgRNA | 1493 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gC1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArGrCrUrCrUrCrCrArGrCrArCrGr |
| | | | | | | GrG*mC*mA*mG |
| MG119-28 sgRNA | 1494 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gD1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrGrGrArGrGrUrCrCrUrCrGrArGr |
| | | | | | | CrG*mC*mG*mG |
| MG119-28 sgRNA | 1495 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gE1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrCrUrUrCrUrCrGrCrUrGrArGrCr |
| | | | | | | GrU*mG*mC*mU |
| MG119-28 sgRNA | 1496 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gF1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrGrUrGrGrUrArCrUrCrGrGrCrCr |
| | | | | | | ArG*mU*mC*mU |
| MG119-28 sgRNA | 1497 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gG1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArGrArGrCrCrUrCrArArGrGrCrGrCr |
| | | | | | | GrC*mG*mG*mC |
| MG119-28 sgRNA | 1498 | 119-28 | Nucleotide | Artificial | | mU*mU*G*rArArArUrArArArArUrGrArArUrUrUrCrArArArCrCrCrCrUrUrCrGrGr |
| targeting hAPOAl | | hAPOA1 | | sequence | | GrGrGrArGrGrGrCrGrCrGrUrUrGrGrArGrCrGrCrCrUrUrArGrUrUrUrGrArGrGrUrGr |
| | | gH1 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrArGrCrUrCrGrUrGrCrArGrCrUrUr |
| | | | | | | CrU*mG*mG*mC |
| MG119-28 sgRNA | 1499 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gA2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArGrCrUrCrUrGrCrGrCrGrCrArGr |
| | | | | | | CrG*mG*mC*mU |
| MG119-28 sgRNA | 1500 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gB2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrCrUrUrCrArCrCrUrCrCrUrCrCr |
| | | | | | | ArG*mA*mU*mC |
| MG119-28 sgRNA | 1501 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gC2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrCrArUrCrUrCrCrUrGrCrCrUrCr |
| | | | | | | ArG*mG*mC*mC |
| MG119-28 sgRNA | 1502 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gD2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrCrCrArGrArArCrUrCrCrUrGrGr |
| | | | | | | GrU*mC*mA*mC |
| MG119-28 sgRNA | 1503 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gE2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrGrArArGrGrUrGrGrArGrGrUrCr |
| | | | | | | ArC*mG*mC*mU |
| MG119-28 sgRNA | 1504 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gF2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArCrArArCrUrGrGrGrArCrArGrCrGr |
| | | | | | | UrG*mA*mC*mC |
| MG119-28 sgRNA | 1505 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gG2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrCrGrCrGrCrGrCrCrUrUrGrArGr |
| | | | | | | GrC*mU*mC*mU |
| MG119-28 sgRNA | 1506 | 119-28 | Nucleotide | Artificial | | mU*mU*mG*rArArArUrArArArArUrGrArArUrUrUrCrArArArCrCrCrCrUrUrCrGrGr |
| targeting hAPOA1 | | hAPOA1 | | sequence | | GrGrGrArGrGrGrCrGrCrGrUrUrGrGrArGrCrGrCrCrUrUrArGrUrUrUrGrArGrGrUrGr |
| | | gH2 | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArGrGrCrUrCrUrCrArArGrGrArGrAr |
| | | | | | | ArC*mG*mG*mC |
| DNA Sequence of | 1507 | 119-28 | Nucleotide | Artificial | | AGCTTCTTAGTGTACTCCTC |
| hAPOA1 Target | | hAPOA1 | | sequence | | |
| Site | | gA1 | | | | |
| DNA Sequence of | 1508 | 119-28 | Nucleotide | Artificial | | GTGTACTCCTCGAGAGCGCT |
| hAPOA1 Target | | hAPOA1 | | sequence | | |
| Site | | gB1 | | | | |
| DNA Sequence of | 1509 | 119-28 | Nucleotide | Artificial | | AAGCTCTCCAGCACGGGCAG |
| hAPOA1 Target | | hAPOAl | | sequence | | |
| Site | | gC1 | | | | |
| DNA Sequence of | 1510 | 119-28 | Nucleotide | Artificial | | GCGGAGGTCCTCGAGCGCGG |
| hAPOAl Target | | hAPOA1 | | sequence | | |
| Site | | gD1 | | | | |
| DNA Sequence of | 1511 | 119-28 | Nucleotide | Artificial | | GCCTTCTCGCTGAGCGTGCT |
| hAPOA1 Target | | hAPOA1 | | sequence | | |
| Site | | gE1 | | | | |
| DNA Sequence of | 1512 | 119-28 | Nucleotide | Artificial | | GCGTGGTACTCGGCCAGTCT |
| hAPOA1 Target | | hAPOA1 | | sequence | | |
| Site | | gF1 | | | | |
| DNA Sequence of | 1513 | 119-28 | Nucleotide | Artificial | | AGAGCCTCAAGGCGCGCGGC |
| hAPOAl Target | | hAPOA1 | | sequence | | |
| Site | | gG1 | | | | |
| DNA Sequence of | 1514 | 119-28 | Nucleotide | Artificial | | CAGCTCGTGCAGCTTCTGGC |
| hAPOAl Target | | hAPOA1 | | sequence | | |
| Site | | gH1 | | | | |
| DNA Sequence of | 1515 | 119-28 | Nucleotide | Artificial | | GAGCTCTGCGCGCAGCGGCT |
| hAPOA1 Target | | hAPOA1 | | sequence | | |
| Site | | gA2 | | | | |
| DNA Sequence of | 1516 | 119-28 | Nucleotide | Artificial | | GCCTTCACCTCCTCCAGATC |
| hAPOA1 Target | | hAPOA1 | | sequence | | |
| Site | | gB2 | | | | |
| DNA Sequence of | 1517 | 119-28 | Nucleotide | Artificial | | CTCATCTCCTGCCTCAGGCC |
| hAPOA1 Target | | hAPOA1 | | sequence | | |
| Site | | gC2 | | | | |
| DNA Sequence of | 1518 | 119-28 | Nucleotide | Artificial | | TCCCAGAACTCCTGGGTCAC |
| hAPOAl Target | | hAPOA1 | | sequence | | |
| Site | | gD2 | | | | |
| DNA Sequence of | 1519 | 119-28 | Nucleotide | Artificial | | CTGAAGGTGGAGGTCACGCT |
| hAPOA1 Target | | hAPOA1 | | sequence | | |
| Site | | gE2 | | | | |
| DNA Sequence of | 1520 | 119-28 | Nucleotide | Artificial | | ACAACTGGGACAGCGTGACC |
| hAPOA1 Target | | hAPOA1 | | sequence | | |
| Site | | gF2 | | | | |
| DNA Sequence of | 1521 | 119-28 | Nucleotide | Artificial | | GCCGCGCGCCTTGAGGCTCT |
| hAPOA1 Target | | hAPOA1 | | sequence | | |
| Site | | gG2 | | | | |
| DNA Sequence of | 1522 | 119-28 | Nucleotide | Artificial | | AGGCTCTCAAGGAGAACGGC |
| hAPOA1 Target | | hAPOA1 | | sequence | | |
| Site | | gH2 | | | | |
| MG119-28 sgRNA | 1523 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gA1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrCrArCrArGrGrUrArArArArCr |
| | | | | | | UrG*mA*mC*mG |
| MG119-28 sgRNA | 1524 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gB1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrGrArCrUrArGrArArArGrGrUrGr |
| | | | | | | ArA*mG*mA*mG |
| MG119-28 sgRNA | 1525 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gC1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArArCrUrCrArGrGrArCrCrArArCr |
| | | | | | | UrU*mA*mU*mU |
| MG119-28 sgRNA | 1526 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gDI | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrCrUrGrArUrUrUrUrGrUrUrUrUr |
| | | | | | | UrC*mC*mA*mA |
| MG119-28 sgRNA | 1527 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gE1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrUrUrUrCrCrArArArCrUrGrCrUr |
| | | | | | | UrC*mU*mC*mC |
| MG119-28 sgRNA | 1528 | 119-28 | Nucleotide | Artificial | | mU*mU*mG*rArArArUrArArArArUrGrArArUrUrUrCrArArArCrCrCrCrUrUrCrGrGrr |
| targeting AAVS1 | | AAVS1 gF1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrArArGrUrGrUrArArGrGrArArGr |
| | | | | | | CrU*mG*mC*mA |
| MG119-28 sgRNA | 1529 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gG1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArGrGrUrUrCrUrGrGrCrArArGrGr |
| | | | | | | ArG*mA*mG*mA |
| MG119-28 sgRNA | 1530 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gH1 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArCrCrCrArArUrArUrCrArGrGrAr |
| | | | | | | GrA*mC*mU*mA |
| MG119-28 sgRNA | 1531 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gA2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArCrCrGrGrCrCrCrUrGrGrGrArArUr |
| | | | | | | ArU*mA*mA*mG |
| MG119-28 sgRNA | 1532 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gB2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArGrGrArArGrArArGrArCrUrArGr |
| | | | | | | CrU*mG*mA*mG |
| MG119-28 sgRNA | 1533 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gC2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrGrUrUrGrUrCrCrArGrArArArAr |
| | | | | | | ArC*mG*mG*mU |
| MG119-28 sgRNA | 1534 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gD2 | | sequence | | GrGrGrArGrGrGrCrGrCrGrUrUrGrGrArGrCrGrCrCrUrUrArGrUrUrUrGrArGrGrUrG |
| | | | | | | CrArGrArArUrCrArArArArArArArCrUrGrCrGrArCrGrArUrGrGrArGrGrUrCrGrUrU |
| | | | | | | UrCrArGrUrCrUrCrUrGrUrArCrArCrUrCrArArArArArArUrUrCrArCrUrUrGrArGrA |
| | | | | | | ArArUrCrArArGrUrGrArArUrArUrCrCrArArCrUrCrCrArGrArArArArArCrGrGrUrG |
| | | | | | | ArU*mG*mA*mU |
| MG119-28 sgRNA | 1535 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gE2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrArUrGrGrCrCrUrUrCrUrCrCrGr |
| | | | | | | ArC*mG*mG*mA |
| MG119-28 sgRNA | 1536 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gF2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrGrUrCrCrCrGrCrCrUrCrCrCrCrUr |
| | | | | | | UrC*mU*mU*mG |
| MG119-28 sgRNA | 1537 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gG2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArGrGrCrCrUrGrCrArUrCrArUrCr |
| | | | | | | ArC*mC*mG*mU |
| MG119-28 sgRNA | 1538 | 119-28 | Nucleotide | Artificial | | mU*U*mG*rArArArUrArArArArUrGrArArUrUrUrCrArArArCrCrCrCrUrUrCrGrGr |
| targeting AAVS1 | | AAVS1 gH2 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrCrArCrCrUrCrUrCrCrArUrCrCr |
| | | | | | | UrC*mU*mU*mG |
| MG119-28 sgRNA | 1539 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gA3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrUrUrCrUrUrUrGrCrCrUrGrGrAr |
| | | | | | | CrA*mC*mC*mC |
| MG119-28 sgRNA | 1540 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gB3 | | sequence | | GrGrGrArGrGrGrCrGrCrGrUrUrGrGrArGrCrGrCrCrUrUrArGrUrUrUrGrArGrGrUrG |
| | | | | | | CrArGrArArUrCrArArArArArArArCrUrGrCrGrArCrGrArUrGrGrArGrGrUrCrGrUrU |
| | | | | | | UrCrArGrUrCrUrCrUrGrUrArCrArCrUrCrArArArArArArUrUrCrArCrUrUrGrArGrAl |
| | | | | | | ArArUrCrArArGrUrGrArArUrArUrCrCrArArCrCrCrUrGrGrArCrArCrCrCrCrGrUrUr |
| | | | | | | CrU*mC*mC*mU |
| MG119-28 sgRNA | 1541 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gC3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrCrArGrCrUrCrCrCrCrUrArCrCr |
| | | | | | | CrC+mC*mC*mU |
| MG119-28 sgRNA | 1542 | 119-28 | Nucleotide | Artificial | | mUmU*mG*rArArArUrArArArArUrGrArArUrUrUrCrArArArCrCrCrCrUrUrCrGrGr |
| targeting AAVS1 | | AAVS1 gD3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrUrCrUrCrUrArGrUrCrUrGrUrGr |
| | | | | | | CrU*mA*mG*mC |
| MG119-28 sgRNA | 1543 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gE3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrGrCrCrUrCrCrArGrGrGrArUrCr |
| | | | | | | CrU*mG*mU*mG |
| MG119-28 sgRNA | 1544 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gF3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArUrUrCrCrCrArGrGrGrCrCrGrGr |
| | | | | | | UrU*mA*mA*mU |
| MG119-28 sgRNA | 1545 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gG3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArUrGrUrGrGrCrUrCrUrGrGrUrUrCr |
| | | | | | | UrG*mG*mG*mU |
| MG119-28 sgRNA | 1546 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gH3 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrGrUrCrCrCrCrUrCrCrArCrCr |
| | | | | | | CrC*mA*mC*mA |
| MG119-28 sgRNA | 1547 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gA4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrUrGrArCrArGrArArArArGrCrCrCr |
| | | | | | | CrA*mU*mC*mC |
| MG119-28 sgRNA | 1548 | 119-28 | Nucleotide | Artificial | | mU*mUmG*rArArArUrArArArArUrGrArArUrUrUrCrArArArCrCrCrCrUrUrCrGrGr |
| targeting AAVS1 | | AAVS1 gB4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrCrCrUrCrCrUrCrCrUrUrCrCrUr |
| | | | | | | ArG*mU*mC*mU |
| MG119-28 sgRNA | 1549 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gC4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrUrCrUrArArCrCrCrCrCrArCrCr |
| | | | | | | UrC*mC*mU*mG |
| MG119-28 sgRNA | 1550 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gD4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrCrArGrArUrUrCrCrUrUrArUrCr |
| | | | | | | UrG*mG*mU*mG |
| MG119-28 sgRNA | 1551 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gE4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrGrGrUrGrArCrArCrArCrCrCr |
| | | | | | | CrC*mA*mU*mU |
| MG119-28 sgRNA | 1552 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gF4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrArGrArArCrCrUrCrUrArArGrGr |
| | | | | | | UrU*mU*mG*mC |
| MG119-28 sgRNA | 1553 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gG4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrUrUrArCrGrArUrGrGrArGrCrCrAr |
| | | | | | | GrA*mG*mA*mG |
| MG119-28 sgRNA | 1554 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gH4 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrGrArUrGrGrArGrCrCrArGrArGrAr |
| | | | | | | GrG*mA*mU*mC |
| MG119-28 sgRNA | 1555 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gA5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrArGrGrGrGrGrUrGrGrGrArGrGr |
| | | | | | | GrA*mA*mG*mG |
| MG119-28 sgRNA | 1556 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gB5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrArCrUrUrCrCrCrArArGrArGrGrAr |
| | | | | | | GrA*mA*mG*mC |
| MG119-28 sgRNA | 1557 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gC5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrArArArArArCrArArArArUrCrArGr |
| | | | | | | ArA*mU*mA*mA |
| MG119-28 sgRNA | 1558 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gD5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrUrCrCrUrGrArGrUrUrCrUrArArCr |
| | | | | | | UrU*mU*mG*mG |
| MG119-28 sgRNA | 1559 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gE5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrCrUrCrUrUrCrArCrCrUrUrUrCrUr |
| | | | | | | ArG*mU*mC*mC |
| MG119-28 sgRNA | 1560 | 119-28 | Nucleotide | Artificial | | mU*mU*G*rArArArUrArArArArUrGrArArUrUrUrCrArArArCrCrCrCrUrUrCrGrGr |
| targeting AAVS1 | | AAVS1 gF5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArUrUrGrUrUrCrCrUrCrCrGrUrGr |
| | | | | | | CrG*mU*mC*mA |
| MG119-28 sgRNA | 1561 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gG5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrUrCrCrUrCrCrGrUrGrCrGrUrCrAr |
| | | | | | | GrU*mU*mU*mU |
| MG119-28 sgRNA | 1562 | 119-28 | Nucleotide | Artificial | | mU*mU*mG*rArArArUrArArArArUrGrArArUrUrUrCrArArArCrCrCrCrUrUrCrGrGr |
| targeting AAVS1 | | AAVS1 gH5 | | sequence | | GrGrGrArGrGrGrCrGrCrGrUrUrGrGrArGrCrGrCrCrUrUrArGrUrUrUrGrArGrGrUrGr |
| | | | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrUrGrUrGrArGrArUrArArGrGrCr |
| | | | | | | CrA*mG*mU*mA |
| DNA Sequence of | 1563 | 119-28 | Nucleotide | Artificial | | TCTCACAGGTAAAACTGACG |
| AAVS1 Target Site | | AAVS1 gA1 | | sequence | | |
| DNA Sequence of | 1564 | 119-28 | Nucleotide | Artificial | | GGGACTAGAAAGGTGAAGAG |
| AAVS1 Target Site | | AAVS1 gB1 | | sequence | | |
| DNA Sequence of | 1565 | 119-28 | Nucleotide | Artificial | | GAACTCAGGACCAACTTATT |
| AAVS1 Target Site | | AAVS1 gC1 | | sequence | | |
| DNA Sequence of | 1566 | 119-28 | Nucleotide | Artificial | | TTCTGATTTTGTTTTTCCAA |
| AAVS1 Target Site | | AAVS1 gDI | | sequence | | |
| DNA Sequence of | 1567 | 119-28 | Nucleotide | Artificial | | TTTTTCCAAACTGCTTCTCC |
| AAVS1 Target Site | | AAVS1 gE1 | | sequence | | |
| DNA Sequence of | 1568 | 119-28 | Nucleotide | Artificial | | GGAAGTGTAAGGAAGCTGCA |
| AAVS1 Target Site | | AAVS1 gF1 | | sequence | | |
| DNA Sequence of | 1569 | 119-28 | Nucleotide | Artificial | | GAGGTTCTGGCAAGGAGAGA |
| AAVS1 Target Site | | AAVS1 gG1 | | sequence | | |
| DNA Sequence of | 1570 | 119-28 | Nucleotide | Artificial | | GACCCAATATCAGGAGACTA |
| AAVS1 Target Site | | AAVS1 gH1 | | sequence | | |
| DNA Sequence of | 1571 | 119-28 | Nucleotide | Artificial | | ACCGGCCCTGGGAATATAAG |
| AAVS1 Target Site | | AAVS1 gA2 | | sequence | | |
| DNA Sequence of | 1572 | 119-28 | Nucleotide | Artificial | | GAGGAAGAAGACTAGCTGAG |
| AAVS1 Target Site | | AAVS1 gB2 | | sequence | | |
| DNA Sequence of | 1573 | 119-28 | Nucleotide | Artificial | | GGGTTGTCCAGAAAAACGGT |
| AAVS1 Target Site | | AAVS1 gC2 | | sequence | | |
| DNA Sequence of | 1574 | 119-28 | Nucleotide | Artificial | | TCCAGAAAAACGGTGATGAT |
| AAVS1 Target Site | | AAVS1 gD2 | | sequence | | |
| DNA Sequence of | 1575 | 119-28 | Nucleotide | Artificial | | GGATGGCCTTCTCCGACGGA |
| AAVS1 Target Site | | AAVS1 gE2 | | sequence | | |
| DNA Sequence of | 1576 | 119-28 | Nucleotide | Artificial | | CGTCCCGCCTCCCCTTCTTG |
| AAVS1 Target Site | | AAVS1 gF2 | | sequence | | |
| DNA Sequence of | 1577 | 119-28 | Nucleotide | Artificial | | TAGGCCTGCATCATCACCGT |
| AAVS1 Target Site | | AAVS1 gG2 | | sequence | | |
| DNA Sequence of | 1578 | 119-28 | Nucleotide | Artificial | | GCCACCTCTCCATCCTCTTG |
| AAVS1 Target Site | | AAVS1 gH2 | | sequence | | |
| DNA Sequence of | 1579 | 119-28 | Nucleotide | Artificial | | CTTTCTTTGCCTGGACACCC |
| AAVS1 Target Site | | AAVS1 gA3 | | sequence | | |
| DNA Sequence of | 1580 | 119-28 | Nucleotide | Artificial | | CCTGGACACCCCGTTCTCCT |
| AAVS1 Target Site | | AAVS1 gB3 | | sequence | | |
| DNA Sequence of | 1581 | 119-28 | Nucleotide | Artificial | | GGCAGCTCCCCTACCCCCCT |
| AAVS1 Target Site | | AAVS1 gC3 | | sequence | | |
| DNA Sequence of | 1582 | 119-28 | Nucleotide | Artificial | | CCTCTCTAGTCTGTGCTAGC |
| AAVS1 Target Site | | AAVS1 gD3 | | sequence | | |
| DNA Sequence of | 1583 | 119-28 | Nucleotide | Artificial | | CTGCCTCCAGGGATCCTGTG |
| AAVS1 Target Site | | AAVS1 gE3 | | sequence | | |
| DNA Sequence of | 1584 | 119-28 | Nucleotide | Artificial | | TATTCCCAGGGCCGGTTAAT |
| AAVS1 Target Site | | AAVS1 gF3 | | sequence | | |
| DNA Sequence of | 1585 | 119-28 | Nucleotide | Artificial | | ATGTGGCTCTGGTTCTGGGT |
| AAVS1 Target Site | | AAVS1 gG3 | | sequence | | |
| DNA Sequence of | 1586 | 119-28 | Nucleotide | Artificial | | TCTGTCCCCTCCACCCCACA |
| AAVS1 Target Site | | AAVS1 gH3 | | sequence | | |
| DNA Sequence of | 1587 | 119-28 | Nucleotide | Artificial | | GTGACAGAAAAGCCCCATCC |
| AAVS1 Target Site | | AAVS1 gA4 | | sequence | | |
| DNA Sequence of | 1588 | 119-28 | Nucleotide | Artificial | | GGCCTCCTCCTTCCTAGTCT |
| AAVS1 Target Site | | AAVS1 gB4 | | sequence | | |
| DNA Sequence of | 1589 | 119-28 | Nucleotide | Artificial | | GGTCTAACCCCCACCTCCTG |
| AAVS1 Target Site | | AAVS1 gC4 | | sequence | | |
| DNA Sequence of | 1590 | 119-28 | Nucleotide | Artificial | | GGCAGATTCCTTATCTGGTG |
| AAVS1 Target Site | | AAVS1 gD4 | | sequence | | |
| DNA Sequence of | 1591 | 119-28 | Nucleotide | Artificial | | TCTGGTGACACACCCCCATT |
| AAVS1 Target Site | | AAVS1 gE4 | | sequence | | |
| DNA Sequence of | 1592 | 119-28 | Nucleotide | Artificial | | CCAGAACCTCTAAGGTTTGC |
| AAVS1 Target Site | | AAVS1 gF4 | | sequence | | |
| DNA Sequence of | 1593 | 119-28 | Nucleotide | Artificial | | CTTACGATGGAGCCAGAGAG |
| AAVS1 Target Site | | AAVS1 gG4 | | sequence | | |
| DNA Sequence of | 1594 | 119-28 | Nucleotide | Artificial | | CGATGGAGCCAGAGAGGATC |
| AAVS1 Target Site | | AAVS1 gH4 | | sequence | | |
| DNA Sequence of | 1595 | 119-28 | Nucleotide | Artificial | | GCAGGGGGTGGGAGGGAAGG |
| AAVS1 Target Site | | AAVS1 gA5 | | sequence | | |
| DNA Sequence of | 1596 | 119-28 | Nucleotide | Artificial | | CACTTCCCAAGAGGAGAAGC |
| AAVS1 Target Site | | AAVS1 gB5 | | sequence | | |
| DNA Sequence of | 1597 | 119-28 | Nucleotide | Artificial | | GAAAAACAAAATCAGAATAA |
| AAVS1 Target Site | | AAVS1 gC5 | | sequence | | |
| DNA Sequence of | 1598 | 119-28 | Nucleotide | Artificial | | GTCCTGAGTTCTAACTTTGG |
| AAVS1 Target Site | | AAVS1 gD5 | | sequence | | |
| DNA Sequence of | 1599 | 119-28 | Nucleotide | Artificial | | GCTCTTCACCTTTCTAGTCC |
| AAVS1 Target Site | | AAVS1 gE5 | | sequence | | |
| DNA Sequence of | 1600 | 119-28 | Nucleotide | Artificial | | TATTGTTCCTCCGTGCGTCA |
| AAVS1 Target Site | | AAVS1 gF5 | | sequence | | |
| DNA Sequence of | 1601 | 119-28 | Nucleotide | Artificial | | TTCCTCCGTGCGTCAGTTTT |
| AAVS1 Target Site | | AAVS1 gG5 | | sequence | | |
| DNA Sequence of | 1602 | 119-28 | Nucleotide | Artificial | | CCTGTGAGATAAGGCCAGTA |
| AAVS1 Target Site | | AAVS1 gH5 | | sequence | | |
| MG119-32 sgRNA | 1603 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gAl | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrArArArCrUrCrUrGrCrArUrCrUrUrUrA*mA*mA*mG |
| MG119-32 sgRNA | 1604 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gB1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrArUrUrUrArUrUrUrCrUrArArArArUrG*mG*mC*mA |
| MG119-32 sgRNA | 1605 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gC1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrGrArCrUrGrUrArArUrUrUrUrCrUrUrU*mU*mG*mC |
| MG119-32 sgRNA | 1606 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gD1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArGrUrGrArCrUrGrArArArCrUrUrCrArC*mA*mG*mA |
| MG119-32 sgRNA | 1607 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gEl | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrCrUrGrUrCrUrUrCrUrUrGrGrUrUrGrC*mU*mG*mU |
| MG119-32 sgRNA | 1608 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gF1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrGrCrUrGrUrUrGrArUrArGrArCrArC*mU*mA*mA |
| MG119-32 sgRNA | 1609 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gG1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrUrArArUrUrGrArArUrUrArUrUrCrUrUrC*mU*mG*mU |
| MG119-32 sgRNA | 1610 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gH1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrArUrUrArUrGrCrArUrUrUrGrUrUrUrCrA*mA*mA*mA |
| MG119-32 sgRNA | 1611 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gA2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArUrArArUrArUrCrUrArArUrArCrUrCrU*mU*mU*mU |
| MG119-32 sgRNA | 1612 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gB2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrGrUrCrUrArUrCrArArCrArGrCrArArC*mC*mA*mA |
| MG119-32 sgRNA | 1613 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gC2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrArUrArGrUrUrArUrGrArArUrUrCrArA*mU*mC*mU |
| MG119-32 sgRNA | 1614 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gD2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrGrCrGrCrArArArArGrArArArArUrUrA*mC*mA*mG |
| MG119-32 sgRNA | 1615 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gE2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArArArUrArArArUrGrCrCrArArArArUrA*mA*mU*mU |
| MG119-32 sgRNA | 1616 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gF2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrUrCrUrArUrUrUrUrCrCrCrArGrUrArArA*mA*mU*mA |
| MG119-32 sgRNA | 1617 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gG2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrCrUrArArArArUrGrGrCrArUrArGrU*mA*mU*mU |
| MG119-32 sgRNA | 1618 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gH2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArUrUrUrGrUrGrArArGrUrCrUrUrArCrA*mA*mG*mG |
| MG119-32 sgRNA | 1619 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gA3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrArArGrUrCrUrUrArCrArArGrGrUrUrA*mU*mC*mU |
| MG119-32 sgRNA | 1620 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gB3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrArArUrArArArArUrUrCrArArArCrArU*mC*mC*mU |
| MG119-32 sgRNA | 1621 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gC3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrArArArArArCrCrArCrArArArArCrC*mU*mG*mU |
| MG119-32 sgRNA | 1622 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gD3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArUrUrUrArUrArUrUrUrArUrUrUrUrCrA*mU*mU*mU |
| MG119-32 sgRNA | 1623 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gE3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrArUrUrUrArUrUrUrUrCrArUrUrUrUrArG*mU*mC*mU |
| MG119-32 sgRNA | 1624 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gF3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrUrCrArUrUrUrUrArGrUrCrUrGrUrC*mU*mU*mC |
| MG119-32 sgRNA | 1625 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gG3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrUrCrUrUrCrArGrUrArUrUrUrArArC*mA*mA*mU |
| MG119-32 sgRNA | 1626 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gH3 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrUrGrGrGrCrArArGrGrGrArArGrArArA*mA*mA*mA |
| MG119-32 sgRNA | 1627 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gA4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArUrUrUrCrUrUrUrCrCrArUrUrUrGrArC*mU*mU*mA |
| MG119-32 sgRNA | 1628 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gB4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrUrUrCrArArArArUrArUrUrGrGrGrCrUrC*mU*mG*mA |
| MG119-32 sgRNA | 1629 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gC4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArGrCrCrUrUrArUrArUrUrCrArArArCrU*mU*mA*mG |
| MG119-32 sgRNA | 1630 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gD4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrArUrUrCrArArArCrUrUrArGrArUrArArU*mA*mU*mC |
| MG119-32 sgRNA | 1631 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gE4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrArGrArUrCrArArCrArGrCrArCrArGrG*mU*mU*mU |
| MG119-32 sgRNA | 1632 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hALB | | hALB gF4 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrGrUrUrUrUrUrArArArUrArArArGrCrA*mU*mA*mG |
| DNA Sequence of | 1633 | 119-32 | Nucleotide | Artificial | | TAAACTCTGCATCTTTAAAG |
| hALB Target Site | | hALB gA1 | | sequence | | |
| DNA Sequence of | 1634 | 119-32 | Nucleotide | Artificial | | CATTTATTTCTAAAATGGCA |
| hALB Target Site | | hALB gB1 | | sequence | | |
| DNA Sequence of | 1635 | 119-32 | Nucleotide | Artificial | | TGACTGTAATTTTCTTTTGC |
| hALB Target Site | | hALB gC1 | | sequence | | |
| DNA Sequence of | 1636 | 119-32 | Nucleotide | Artificial | | AGTGACTGAAACTTCACAGA |
| hALB Target Site | | hALB gD1 | | sequence | | |
| DNA Sequence of | 1637 | 119-32 | Nucleotide | Artificial | | TCTGTCTTCTTGGTTGCTGT |
| hALB Target Site | | hALB gE1 | | sequence | | |
| DNA Sequence of | 1638 | 119-32 | Nucleotide | Artificial | | TTGCTGTTGATAGACACTAA |
| hALB Target Site | | hALB gF1 | | sequence | | |
| DNA Sequence of | 1639 | 119-32 | Nucleotide | Artificial | | TAATTGAATTATTCTTCTGT |
| hALB Target Site | | hALB gG1 | | sequence | | |
| DNA Sequence of | 1640 | 119-32 | Nucleotide | Artificial | | ATTATGCATTTGTTTCAAAA |
| hALB Target Site | | hALB gH1 | | sequence | | |
| DNA Sequence of | 1641 | 119-32 | Nucleotide | Artificial | | ATAATATCTAATACTCTTTT |
| hALB Target Site | | hALB gA2 | | sequence | | |
| DNA Sequence of | 1642 | 119-32 | Nucleotide | Artificial | | TGTCTATCAACAGCAACCAA |
| hALB Target Site | | hALB gB2 | | sequence | | |
| DNA Sequence of | 1643 | 119-32 | Nucleotide | Artificial | | GATAGTTATGAATTCAATCT |
| hALB Target Site | | hALB gC2 | | sequence | | |
| DNA Sequence of | 1644 | 119-32 | Nucleotide | Artificial | | TGCGCAAAAGAAAATTACAG |
| hALB Target Site | | hALB gD2 | | sequence | | |
| DNA Sequence of | 1645 | 119-32 | Nucleotide | Artificial | | AAATAAATGCCAAAATAATT |
| hALB Target Site | | hALB gE2 | | sequence | | |
| DNA Sequence of | 1646 | 119-32 | Nucleotide | Artificial | | TCTATTTTCCCAGTAAAATA |
| hALB Target Site | | hALB gF2 | | sequence | | |
| DNA Sequence of | 1647 | 119-32 | Nucleotide | Artificial | | TTCTAAAATGGCATAGTATT |
| hALB Target Site | | hALB gG2 | | sequence | | |
| DNA Sequence of | 1648 | 119-32 | Nucleotide | Artificial | | ATTTGTGAAGTCTTACAAGG |
| hALB Target Site | | hALB gH2 | | sequence | | |
| DNA Sequence of | 1649 | 119-32 | Nucleotide | Artificial | | GAAGTCTTACAAGGTTATCT |
| hALB Target Site | | hALB gA3 | | sequence | | |
| DNA Sequence of | 1650 | 119-32 | Nucleotide | Artificial | | TAATAAAATTCAAACATCCT |
| hALB Target Site | | hALB gB3 | | sequence | | |
| DNA Sequence of | 1651 | 119-32 | Nucleotide | Artificial | | TTAAAAACCACAAAACCTGT |
| hALB Target Site | | hALB gC3 | | sequence | | |
| DNA Sequence of | 1652 | 119-32 | Nucleotide | Artificial | | ATTTATATTTATTTTCATTT |
| hALB Target Site | | hALB gD3 | | sequence | | |
| DNA Sequence of | 1653 | 119-32 | Nucleotide | Artificial | | ATTTATTTTCATTTTAGTCT |
| hALB Target Site | | hALB gE3 | | sequence | | |
| DNA Sequence of | 1654 | 119-32 | Nucleotide | Artificial | | TTTCATTTTAGTCTGTCTTC |
| hALB Target Site | | hALB gF3 | | sequence | | |
| DNA Sequence of | 1655 | 119-32 | Nucleotide | Artificial | | TTTCTTCAGTATTTAACAAT |
| hALB Target Site | | hALB gG3 | | sequence | | |
| DNA Sequence of | 1656 | 119-32 | Nucleotide | Artificial | | CTGGGCAAGGGAAGAAAAAA |
| hALB Target Site | | hALB gH3 | | sequence | | |
| DNA Sequence of | 1657 | 119-32 | Nucleotide | Artificial | | ATTTCTTTCCATTTGACTTA |
| hALB Target Site | | hALB gA4 | | sequence | | |
| DNA Sequence of | 1658 | 119-32 | Nucleotide | Artificial | | TTCAAAATATTGGGCTCTGA |
| hALB Target Site | | hALB gB4 | | sequence | | |
| DNA Sequence of | 1659 | 119-32 | Nucleotide | Artificial | | AGCCTTATATTCAAACTTAG |
| hALB Target Site | | hALB gC4 | | sequence | | |
| DNA Sequence of | 1660 | 119-32 | Nucleotide | Artificial | | ATTCAAACTTAGATAATATC |
| hALB Target Site | | hALB gD4 | | sequence | | |
| DNA Sequence of | 1661 | 119-32 | Nucleotide | Artificial | | GAGATCAACAGCACAGGTTT |
| hALB Target Site | | hALB gE4 | | sequence | | |
| DNA Sequence of | 1662 | 119-32 | Nucleotide | Artificial | | GGTTTTTAAATAAAGCATAG |
| hALB Target Site | | hALB gF4 | | sequence | | |
| MG119-32 sgRNA | 1663 | 119-32 | Nucleotide | Artificial | | mA*mU*mA*rUrArGrUrCrCrCrArUrUrArGrGrGrArUrUrGrGrGrCrGrUrGrUrUrGrG |
| targeting hAPOA1 | | hAPOA1 | | sequence | | ArArCrGrCrCrUrUrArGrUrUrUrGrArGrGrUrUrUrGrArArArCrCrArArArArGrCrGrG |
| | | gA1 | | | | UrGrUrArGrArUrGrGrArGrGrUrCrUrGrUrArCrCrArCrCrUrUrCrArArGrCrArCrUrC |
| | | | | | | ArArArArArArUrCrCrArCrUrUrArUrArUrGrArArArArUrArUrArArGrGrGrGrArUrA |
| | | | | | | UrCrCrArArCrGrUrGrUrArCrUrCrCrUrCrGrArGrArGrCrG*mC*mU*mC |
| MG119-32 sgRNA | 1664 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOA1 | | hAPOA1 | | sequence | | ArArCrGrCrCrUrUrArGrUrUrUrGrArGrGrUrUrUrGrArArArCrCrArArArArGrCrGrGr |
| | | gB1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrGrGrArGrGrUrCrCrUrCrGrArGrCrGrC*mG*mG*mG |
| MG119-32 sgRNA | 1665 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOA1 | | hAPOA1 | | sequence | | ArArCrGrCrCrUrUrArGrUrUrUrGrArGrGrUrUrUrGrArArArCrCrArArArArGrCrGrGr |
| | | gC1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrCrUrUrCrUrCrGrCrUrGrArGrCrGrUrG*mC*mU*mC |
| MG119-32 sgRNA | 1666 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOA1 | | hAPOA1 | | sequence | | ArArCrGrCrCrUrUrArGrUrUrUrGrArGrGrUrUrUrGrArArArCrCrArArArArGrCrGrGr |
| | | gD1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrGrUrGrGrUrArCrUrCrGrGrCrCrArGrU*mC*mU*mG |
| MG119-32 sgRNA | 1667 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOAl | | hAPOA1 | | sequence | | ArArCrGrCrCrUrUrArGrUrUrUrGrArGrGrUrUrUrGrArArArCrCrArArArArGrCrGrGr |
| | | gE1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArGrCrUrCrUrGrCrGrCrGrCrArGrCrGrG*mC*mU*mC |
| MG119-32 sgRNA | 1668 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOA1 | | hAPOA1 | | sequence | | ArArCrGrCrCrUrUrArGrUrUrUrGrArGrGrUrUrUrGrArArArCrCrArArArArGrCrGrGr |
| | | gF1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrCrUrUrCrArCrCrUrCrCrUrCrCrArGrA*mU*mC*mC |
| MG119-32 sgRNA | 1669 | 119-32 | Nucleotide | Artificial | | mA*mU*mA*rUrArGrUrCrCrCrArUrUrArGrGrGrArUrUrGrGrGrCrGrUrGrUrUrGrGr |
| targeting hAPOA1 | | hAPOA1 | | sequence | | ArArCrGrCrCrUrUrArGrUrUrUrGrArGrGrUrUrUrGrArArArCrCrArArArArGrCrGrGr |
| | | gG1 | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrCrGrCrGrCrGrCrCrUrUrGrArGrGrCrU*mC*mU*mC |
| DNA Sequence of | 1670 | 119-32 | Nucleotide | Artificial | | TGTACTCCTCGAGAGCGCTC |
| hAPOA1 Target | | hAPOA1 | | sequence | | |
| Site | | gAl | | | | |
| DNA Sequence of | 1671 | 119-32 | Nucleotide | Artificial | | CGGAGGTCCTCGAGCGCGGG |
| hAPOA1 Target | | hAPOA1 | | sequence | | |
| Site | | gB1 | | | | |
| DNA Sequence of | 1672 | 119-32 | Nucleotide | Artificial | | CCTTCTCGCTGAGCGTGCTC |
| hAPOA1 Target | | hAPOA1 | | sequence | | |
| Site | | gC1 | | | | |
| DNA Sequence of | 1673 | 119-32 | Nucleotide | Artificial | | CGTGGTACTCGGCCAGTCTG |
| hAPOAl Target | | hAPOAl | | sequence | | |
| Site | | gD1 | | | | |
| DNA Sequence of | 1674 | 119-32 | Nucleotide | Artificial | | AGCTCTGCGCGCAGCGGCTC |
| hAPOAl Target | | hAPOA1 | | sequence | | |
| Site | | gEl | | | | |
| DNA Sequence of | 1675 | 119-32 | Nucleotide | Artificial | | CCTTCACCTCCTCCAGATCC |
| hAPOA1 Target | | hAPOA1 | | sequence | | |
| Site | | gF1 | | | | |
| DNA Sequence of | 1676 | 119-32 | Nucleotide | Artificial | | CCGCGCGCCTTGAGGCTCTC |
| hAPOA1 Target | | hAPOA1 | | sequence | | |
| Site | | gG1 | | | | |
| MG119-32 sgRNA | 1677 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gAl | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrArArGrUrGrUrArArGrGrArArGrCrUrG*mC*mA*mG |
| MG119-32 sgRNA | 1678 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gB1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArGrGrUrUrCrUrGrGrCrArArGrGrArGrA*mG*mA*mG |
| MG119-32 sgRNA | 1679 | 119-32 | Nucleotide | Artificial | | mA*mlJ*mA*rlJrArGrUrCrCrCrArUrUrArGrGrGrArUrUrGrGrGrCrGrUrGrUrUrGrGr |
| targeting AAVS1 | | AAVS1 gC1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrGrUrUrGrUrCrCrArGrArArArArArCrG*mG*mU*mG |
| MG119-32 sgRNA | 1680 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gD1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrA |
| | | | | | | UrCrCrArArCrGrGrArUrGrGrCrCrUrUrCrUrCrCrGrArCrG*mG*mA*mU |
| MG119-32 sgRNA | 1681 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gE1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrCrArCrCrUrCrUrCrCrArUrCrCrUrCrU*mU*mG*mC |
| MG119-32 sgRNA | 1682 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gF1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrCrArGrCrUrCrCrCrCrUrArCrCrCrCrC*mC*mU*mU |
| MG119-32 sgRNA | 1683 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gG1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrGrCrCrUrCrCrUrCrCrUrUrCrCrUrArGrU*C*mU*mC |
| MG119-32 sgRNA | 1684 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gH1 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrArGrGrGrGrGrUrGrGrGrArGrGrGrArA*mG*mG*mG |
| MG119-32 sgRNA | 1685 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gA2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrArArArArArCrArArArArUrCrArGrArArU*mA*mA*mG |
| MG119-32 sgRNA | 1686 | 119-32 | Nucleotide | Artificial | | mA*mU*mA*rUrArGrUrCrCrCrArUrUrArGrGrGrArUrUrGrGrGrCrGrUrGrUrUrGrGr |
| targeting AAVS1 | | AAVS1 gB2 | | sequence | | ArArCrGrCrCrUrUrArGrUrUrUrGrArGrGrUrUrUrGrArArArCrCrArArArArGrCrGrGr |
| | | | | | | UrGrUrArGrArUrGrGrArGrGrUrCrUrGrUrArCrCrArCrCrUrUrCrArArGrCrArCrUrCr |
| | | | | | | ArArArArArArUrCrCrArCrUrUrArUrArUrGrArArArArUrArUrArArGrGrGrGrArUrAr |
| | | | | | | UrCrCrArArCrGrCrUrCrUrUrCrArCrCrUrUrUrCrUrArGrU*mC*mC*mC |
| DNA Sequence of | 1687 | 119-32 | Nucleotide | Artificial | | GAAGTGTAAGGAAGCTGCAG |
| AAVS1 Target Site | | AAVS1 gA1 | | sequence | | |
| DNA Sequence of | 1688 | 119-32 | Nucleotide | Artificial | | AGGTTCTGGCAAGGAGAGAG |
| AAVS1 Target Site | | AAVS1 gB1 | | sequence | | |
| DNA Sequence of | 1689 | 119-32 | Nucleotide | Artificial | | GGTTGTCCAGAAAAACGGTG |
| AAVS1 Target Site | | AAVS1 gC1 | | sequence | | |
| DNA Sequence of | 1690 | 119-32 | Nucleotide | Artificial | | GATGGCCTTCTCCGACGGAT |
| AAVS1 Target Site | | AAVS1 gD1 | | sequence | | |
| DNA Sequence of | 1691 | 119-32 | Nucleotide | Artificial | | CCACCTCTCCATCCTCTTGC |
| AAVS1 Target Site | | AAVS1 gEl | | sequence | | |
| DNA Sequence of | 1692 | 119-32 | Nucleotide | Artificial | | GCAGCTCCCCTACCCCCCTT |
| AAVS1 Target Site | | AAVS1 gF1 | | sequence | | |
| DNA Sequence of | 1693 | 119-32 | Nucleotide | Artificial | | GCCTCCTCCTTCCTAGTCTC |
| AAVS1 Target Site | | AAVS1 gG1 | | sequence | | |
| DNA Sequence of | 1694 | 119-32 | Nucleotide | Artificial | | CAGGGGGTGGGAGGGAAGGG |
| AAVS1 Target Site | | AAVS1 gH1 | | sequence | | |
| DNA Sequence of | 1695 | 119-32 | Nucleotide | Artificial | | AAAAACAAAATCAGAATAAG |
| AAVS1 Target Site | | AAVS1 gA2 | | sequence | | |
| DNA Sequence of | 1696 | 119-32 | Nucleotide | Artificial | | CTCTTCACCTTTCTAGTCCC |
| AAVS1 Target Site | | AAVS1 gB2 | | sequence | | |
| MG119-2_sg2_WT | 1697 | MG119- | Nucleotide | Artificial | | UACACAUCAUUAGAUGUGAGGGCAAUUUAAUGUCCUAAUAUUGAGGGUAU |
| split guide | | 2_sg2_WT_ | | sequence | | UAGGUAACAACACUUUGUUCUA |
| | | Av1 | | | | |
| MG119-2_sg2_WT | 1698 | MG119- | Nucleotide | Artificial | U40 spacer | AUAGAACAGUGUCUAAUAACCACUCGAUAAGACCAUAUAAAUGAAAAUUU |
| split guide | | 2_sg2_WT_ | | sequence | | GUAUGGUUAUAAAAC |
| | | Bv1 | | | | |
| MG119-2_sg2_WT | 1699 | MG119- | Nucleotide | Artificial | | UACACAUCAUUAGAUGUGAGGGCAAUUUAAUGUCCUAAUAUUGAGGGUAU |
| split guide | | 2_sg2_WT | | sequence | | UAGGUAACAACACUUUGUUC |
| | | Av2/4 | | | | |
| MG119-2_sg2_WT | 1700 | MG119- | Nucleotide | Artificial | U40 spacer | UAAUAGAACAGUGUCUAAUAACCACUCGAUAAGACCAUAUAAAUGAAAAU |
| split guide | | 2_sg2_WT_ | | sequence | | UUGUAUGGUUAUAAAAC |
| | | Bv2 | | | | |
| MG119-2_sg2_WT | 1701 | MG119- | Nucleotide | Artificial | | UACACAUCAUUAGAUGUGAGGGCAAUUUAAUGUCCUAAUAUUGAGGGUAU |
| split guide | | 2_sg2_WT_ | | sequence | | UAGGUAACAACACUUUGUUCUAAUA |
| | | Av3 | | | | |
| MG119-2_sg2_WT | 1702 | MG119- | Nucleotide | Artificial | U40 spacer | GAACAGUGUCUAAUAACCACUCGAUAAGACCAUAUAAAUGAAAAUUUGU |
| split guide | | 2_sg2_WT_ | | sequence | | UGGUUAUAAAAC |
| | | Bv3/4 | | | | |
| MG119-2_sg2_WT | 1703 | MG119- | Nucleotide | Artificial | | UACACAUCAUUAGAUGUGAGGGCAAUUUAAUGUCCUAAUAUUGAGGGUAI |
| split guide | | 2_sg2_WT_ | | sequence | | UAGGUAACAACACUUUGUUCUAAUAGAACAGUGUCUAAU |
| | | Av5 | | | | |
| MG119-2_sg2_WT | 1704 | MG119- | Nucleotide | Artificial | U40 spacer | ACCACUCGAUAAGACCAUAUAAAUGAAAAUUUGUAUGGUUAUAAAAC |
| split guide | | 2_sg2_WT_ | | sequence | | |
| | | Bv5 | | | | |
| MG119- | 1705 | MG119- | Nucleotide | Artificial | | UACGAGGGCAAUUUAAUGUCCUAAUAUUGAGGGUAUUAGGUAACA |
| 2_sg2_4.6.11 split | | 2_sg2_4.6.1 | | sequence | | |
| guide | | 1 Av1 | | | | |
| MG119- | 1706 | MG119- | Nucleotide | Artificial | U40 spacer | ACAUGUCUAAUAACCACUCGAUAAGACCAUAUGAAAGUAUGGUUAUAAAA |
| 2_sg2_4.6.11 split | | 2_sg2_4.6.1 | | sequence | | C |
| guide | | 1 Bvl | | | | |
| MG119- | 1707 | MG119- | Nucleotide | Artificial | | UACGAGGGCAAUUUAAUGUCCUAAUAUUGAGGGUAUUAGG |
| 2_sg2_4.6.11 split | | 2_sg2_4.6.1 | | sequence | | |
| guide | | 1 Av2/4 | | | | |
| MG119- | 1708 | MG119- | Nucleotide | Artificial | c | UAACAACAUGUCUAAUAACCACUCGAUAAGACCAUAUGAAAGUAUGGUUA |
| 2_sg2_4.6.11 split | | 2_sg2_4.6.1 | | sequence | | UAAAAC |
| guide | | 1 Bv2 | | | | |
| MG119- | 1709 | MG119- | Nucleotide | Artificial | | UACGAGGGCAAUUUAAUGUCCUAAUAUUGAGGGUAUUAGGUAACAACAUG |
| 2_sg2_4.6.11 split | | 2_sg2_4.6.1 | | sequence | | |
| guide | | 1 Av3 | | | | |
| MG119- | 1710 | MG119- | Nucleotide | Artificial | U40 spacer | CCUAAUAACCACUCGAUAAGACCAUAUGAAAGUAUGGUUAUAAAAC |
| 2_sg2_4.6.11 split | | 2_sg2_4.6.1 | | sequence | | |
| guide | | 1 Bv3/4 | | | | |
| MG119- | 1711 | MG119- | Nucleotide | Artificial | | UACGAGGGCAAUUUAAUGUCCUAAUAUUGAGGGUAUUAGGUAACAACAUG |
| 2_sg2_4.6.11 split | | 2_sg2_4.6.1 | | sequence | | UCUAAU |
| guide | | 1 Av5 | | | | |
| MG119- | 1712 | MG119- | Nucleotide | Artificial | U40 spacer | ACCACUCGAUAAGACCAUAUGAAAGUAUGGUUAUAAAAC |
| 2_sg2_4.6.11 split | | 2_sg2_4.6.1 | | sequence | | |
| guide | | Bv5 | | | | |
| MG119- | 1713 | MG119- | Nucleotide | Artificial | | CAAACCCCUUCGGGGGAGGGCGCGUUGGAGCGCCUUAGUUUGAGGUGCAG |
| 28_sg1_WT split | | 28_sg1_WT | | sequence | | AAUCAAAAAAACUGCGACGAUGG |
| guide | | Av1 | | | | |
| MG119- | 1714 | MG119- | Nucleotide | Artificial | U40 spacer | AGGUCGUUUCAGUCUCUGUACACUCAAAAAAUUCACUUGAGAAAUCAAGU |
| 28_sg1_WT split | | 28_sg1_WT | | sequence | | GAAUAUCCAAC |
| guide | | Bv1 | | | | |
| MG119- | 1715 | MG119- | Nucleotide | Artificial | | CAAACCCCUUCGGGGGAGGGCGCGUUGGAGCGCCUUAGUUUGAGGUGCAG |
| 28_sg1_WT split | | 28_sg1_WT | | sequence | | AAUCAAAAAAACUGCGACGAC |
| guide | | Av2 | | | | |
| MG119- | 1716 | MG119- | Nucleotide | Artificial | U40 spacer | GUCGUUUCAGUCUCUGUACACUCAAAAAAUUCACUUGAGAAAUCAAGUGA |
| 28_sg1_WT split | | 28_sg1_WT | | sequence | | AUAUCCAAC |
| guide | | Bv2 | | | | |
| MG119- | 1717 | MG119- | Nucleotide | Artificial | | CAAACCCCUUCGGGGGAGGGCGCGUUGGAGCGCCUUAGUUUGAGGUGCAG |
| 28_sg1_WT split | | 28_sg1_WT | | sequence | | AAUCAAAAAAACUGCGACGAUGGAGGUCGUC |
| guide | | Av3 | | | | |
| MG119- | 1718 | MG119- | Nucleotide | Artificial | U40 spacer | CAGUCUCUGUACACUCAAAAAAUUCACUUGAGAAAUCAAGUGAAUAUCCA |
| 28_sg1_WT split | | 28_sg1_WT | | sequence | | AC |
| guide | | Bv3 | | | | |
| MG119- | 1719 | MG119- | Nucleotide | Artificial | | CAAACCCCUUCGGGGGAGGGCGCGUUGGAGCGCCUUAGUUUGAGGUGCAG |
| 28_sg1_WT split | | 28_sg1_WT | | sequence | | A |
| guide | | Av4 | | | | |
| MG119- | 1720 | MG119- | Nucleotide | Artificial | U40 spacer | AUCAAAAAAACUGCGACGAUGGAGGUCGUUUCAGUCUCUGUACACUCAAA |
| 28_sg1_WT split | | 28_sg1_WT | | sequence | | AAAUUCACUUGAGAAAUCAAGUGAAUAUCCAAC |
| guide | | Bv4 | | | | |
| MG119-28_sg1_8.5 | 1721 | MG119- | Nucleotide | Artificial | | CAGGGCGCGUUGGAGCGCCUUAGUUUGAGGUGCAGAAUCAAAAAAACUGC |
| split guide | | 28_sg1_8.5 | | sequence | | GACGAUGG |
| | | Av1 | | | | |
| MG119-28_sg1_8.5 | 1722 | MG119- | Nucleotide | Artificial | U40 spacer | AGGUCGUUUCAGUCUCUGUACACUCAAAAAAUAUCCAAC |
| split guide | | 28_sg1_8.5 | | sequence | | |
| | | Bv1 | | | | |
| MG119-28_sg1_8.5 | 1723 | MG119- | Nucleotide | Artificial | | CAGGGCGCGUUGGAGCGCCUUAGUUUGAGGUGCAGAAUCAAAAAAACUGC |
| split guide | | 28_sg1_8.5 | | sequence | | GACGAC |
| | | Av2 | | | | |
| MG119-28_sg1_8.5 | 1724 | MG119- | Nucleotide | Artificial | U40 spacer | GUCGUUUCAGUCUCUGUACACUCAAAAAAUAUCCAAC |
| split guide | | 28_sg1_8.5 | | sequence | | |
| | | Bv2 | | | | |
| MG119-28_sg1_8.5 | 1725 | MG119- | Nucleotide | Artificial | | CAGGGCGCGUUGGAGCGCCUUAGUUUGAGGUGCAGAAUCAAAAAAACUGC |
| split guide | | 28_sg1_8.5 | | sequence | | GACGAUGGAGGUCGUC |
| | | Av3 | | | | |
| MG119-28_sg1_8.5 | 1726 | MG119- | Nucleotide | Artificial | U40 spacer | CAGUCUCUGUACACUCAAAAAAUAUCCAAC |
| split guide | | 28_sg1_8.5 | | sequence | | |
| | | Bv3 | | | | |
| MG119-28_sg1_8.5 | 1727 | MG119- | Nucleotide | Artificial | | CAGGGCGCGUUGGAGCGCCUUAGUUUGAGGUGCAGA |
| split guide | | 28_sg1_8.5_ | | sequence | | |
| | | Av4 | | | | |
| MG119-28_sg1_8.5 | 1728 | MG119- | Nucleotide | Artificial | U40 spacer | AUCAAAAAAACUGCGACGAUGGAGGUCGUUUCAGUCUCUGUACACUCAAA |
| split guide | | 28_sg1_8.5 | | sequence | | AAAUAUCCAAC |
| | | Bv4 | | | | |
| MG119-32_sg1_R2 | 1729 | MG119- | Nucleotide | Artificial | U40 spacer | AUAUGGGCGUGUUGGAACGCCUUAGUUUGAGGUUUGAAACCAAAAGCGGU |
| truncation | | 32_sg1_5.6 | | sequence | | GUAGAUGGAGGUCUGUACCACCUUCAAGCACUCAAAAAACUUAUAUGAAA |
| | | | | | | AUAUAAGUAUCCAACG |
| MG119-32_sg1_R2 | 1730 | MG119- | Nucleotide | Artificial | U40 spacer | AUAUGGGCGUGUUGGAACGCCUUAGUUUGAGGUUUGAAACCAAAAGCGGU |
| truncation | | 32_sg1_5.7 | | sequence | | GUAGAUGGAGGUCUGUACCACCUUCAAGCACUCAAAAAAUCCGAAAGGAU |
| | | | | | | AUCCAACG |
| MG119-32_sg1_R2 | 1731 | MG119- | Nucleotide | Artificial | U40 spacer | AUAUGGGCGUGUUGGAACGCCUUAGUUUGAGGUUUGAAACCAAAAGCGGU |
| truncation | | 32_sg1_5.8 | | sequence | | GUAGAUGGAGGUCUGUACCACCUUCAAGCACUCAAAAAAUAUCCAACG |
| MG191-15 Minimal | 1732 | MG191- | Nucleotide | Artificial | | GAAGTGCCATTCCGCCTGACCTTAATACGACTCACTATAGATTGTTGAATAA |
| Array | | 15_5U40_3 | | sequence | | TTCCTTTTATAGGGTTGTTACAACTGGAGATATCTTGAACCTTGCATCCCCGG |
| | | 0_R | | | | AATTGTTGAATAATTCCTTTTATAGGGTTGTTACAACAGGCTAGGTGGAGGC |
| | | | | | | TCAGTG |
| MG191-17 Minimal | 1733 | MG191- | Nucleotide | Artificial | | GAAGTGCCATTCCGCCTGACCTTAATACGACTCACTATAGATTGTTGTAATCC |
| Array | | 17_5U40_3 | | sequence | | ACATTTTGTATGGGATTAACAACTGGAGATATCTTGAACCTTGCATCCCCGG |
| | | 0_F | | | | AATTGTTGTAATCCACATTTTGTATGGGATTAACAACAGGCTAGGTGGAGGC |
| | | | | | | TCAGTG |
| MG191-18 Minimal | 1734 | MG191- | Nucleotide | Artificial | | GAAGTGCCATTCCGCCTGACCTTAATACGACTCACTATAGATTGTTGGAATA |
| Array | | 18_5U40_3 | | sequence | | CATCTTTTTTAGGGTATTTACAACTGGAGATATCTTGAACCTTGCATCCCCGG |
| | | 0_F | | | | AATTGTTGGAATACATCTTTTTTAGGGTATTTACAACAGGCTAGGTGGAGGC |
| | | | | | | TCAGTG |
| MG191-22 Minimal | 1735 | MG191- | Nucleotide | Artificial | | GAAGTGCCATTCCGCCTGACCTTAATACGACTCACTATAGATTGTTGGTTATC |
| Array | | 22_5U40_3 | | sequence | | CTAATTTCATAGGAATACACAACTGGAGATATCTTGAACCTTGCATCCCCGG |
| | | 0_F | | | | AATTGTTGGTTATCCTAATTTCATAGGAATACACAACAGGCTAGGTGGAGGC |
| | | | | | | TCAGTG |
| MG191-23 Minimal | 1736 | MG191- | Nucleotide | Artificial | | GAAGTGCCATTCCGCCTGACCTTAATACGACTCACTATAGATTGTTGTTACAT |
| Array | | 23_5U40_3 | | sequence | | CATTTTTATAAGGTGTAAACAACTGGAGATATCTTGAACCTTGCATCCCCGG [symbol] |
| | | 0_F | | | | AATTGTTGTTACATCATTTTTATAAGGTGTAAACAACAGGCTAGGTGGAGGC |
| | | | | | | TCAGTG |
| MG191-25 Minimal | 1737 | MG191- | Nucleotide | Artificial | | GAAGTGCCATTCCGCCTGACCTTAATACGACTCACTATAGATTGGTGGAGTT |
| Array | | 25_5U40_3 | | sequence | | CGCATTCTATATCCTACTTACACCTGGAGATATCTTGAACCTTGCATCCCCG [symbol] |
| | | 0_F | | | | AATTGGTGGAGTTCGCATTCTATATCCTACTTACACCAGGCTAGGTGGAGGC [symbol] |
| | | | | | | TCAGTG |
| MG191-29 Minimal | 1738 | MG191- | Nucleotide | Artificial | | GAAGTGCCATTCCGCCTGACCTTAATACGACTCACTATAGCTTGTTGTAATT [symbol] |
| Array | | 29_5U40_3 | | sequence | | CTCTTTTTTAGGTAATAAACAACTGGAGATATCTTGAACCTTGCATCCCCGG [symbol] |
| | | 0_F | | | | CTTGTTGTAATTACTCTTTTTTAGGTAATAAACAACAGGCTAGGTGGAGGCT [symbol] |
| | | | | | | AGTG |
| MG191-15 | | MG191-15 | Nucleotide | Artificial | | tTRR |
| | | PAM | | sequence | | |
| MG191-17 | | MG191-17 | Nucleotide | Artificial | | TYR |
| | | PAM | | sequence | | |
| MG191-18 | | MG191-18 | Nucleotide | Artificial | | TRR |
| | | PAM | | sequence | | |
| MG191-22 | | MG191-22 | Nucleotide | Artificial | | tYR |
| | | PAM | | sequence | | |
| MG191-23 | | MG191-23 | Nucleotide | Artificial | | nCn |
| | | PAM | | sequence | | |
| MG191-25 | | MG191-25 | Nucleotide | Artificial | | TnR |
| | | PAM | | sequence | | |
| MG191-29 | | MG191-29 | Nucleotide | Artificial | | TAG |
| | | PAM | | sequence | | |
| MG185-1 effector | 1746 | MG185-1 | Protein | Unclassi- | Uncultivated | MVEAVKAGVDSDRREGGPRRLRLRLVPSPRQECLFERAGRVVRELEDCVRELR |
| | | effector | | fied | organism | DLTLAWFEADAAGDRLVFRFRPRHAIRPGLYLDPQRFSGESDDAYQKRRGEQR |
| | | | | | | DRVVRYLVGEQHSERESGERGRSWLLPRPDADYWRKVDGSVRGWRLKVLSAA |
| | | | | | | RMAQPCRRVPVLICHQGGTQRAGVAAFSARPIREERGVEPVLWRASDPQGKPH |
| | | | | | | GNRKEDERDIARSHIQRVEPQHVFDQGAVDACGNRISSVYASLTDDEVSVGLAA |
| | | | | | | HTRNAVLRVIAATELDRKRRGRGNVQSYGEHWVPSGWWRSRNPGKARRVERR |
| | | | | | | AEARTVSIPLHKQQREFLSAKRDKWRAGGDGEFKRARRLLVEFGSLVVDGGGY |
| | | | | | | SGKVRVRAWSPYNPNTHCPGECTLIRDGAGRWYLSVVLRKPPPQRTASVGERR |
| | | | | | | GECGIDLGARHTAAVAFEDPGSGTPKYRKIELPPDLHKRLRNLTSLSEGSGRLRQ |
| | | | | | | LRRDPLARLYDMERLYSHKLRIGNPDALEPVPGRPGRKRLRRGAAPRKSKRALR |
| | | | | | | LERAMARQRSRVEALVTSHYNREAHKLCAEFERIGVEDLNLGQQRHQVPDVGP |
| | | | | | | GGKRIGRAARARMIRVLQLASPGKLRRAIEEKSALYGTEVEPVDPRGTSKTCPH [symbol] |
| | | | | | | REEQLERYPDNTGRCASCGKHVNNVDESAALEILRRMREPGSSHNSRGALAKR |
| | | | | | | ANQASTVAPAALATPSSDSHRGVSRGAEPAGLCDRSQNTPQTRTTTAQRDPPA* |
| MG186-1 effector | 1747 | MG186-1 | Protein | Unclassi- | Uncultivated | MILPIMAEKKKKQSKNDGRILLGVPMALQTEKLSKNTRMNGKELLRSQVGSLE [symbol] |
| | | effector | | fied | organism | HAALSRQVYNWVTALYLSFLKMKDDYIREKGLGYANNDEQALCSLKASVYA [symbol] |
| | | | | | | KOKTKNDEDKKGNKKKRTKPKDSDPVPLSAKIIEDFTHVDAVTGEVFVPTSVTY |
| | | | | | | QEAKEFDSQTASFYKQFNEEHAYDAPEQKHPDRNPRKLPSWILKKQTIEYVYSQ |
| | | | | | | VGSVKEDTHRFVGNVLYDSLADSNVLPQWFTNLVIEQESLHPLYRYDNRSVWV |
| | | | | | | PVGEDENIEMFPVPRRVAVNEIENACNAVEKHRKRFFKGGNPKQGKPKFRKYK |
| | | | | | | DDQTFAISMPDLFLRQGISAAAKNKFLSGISLDSSMGEDARREAEGKLWAEEKH |
| | | | | | | KRKTSWDRLASGSRIRLGYLGNVPVSKHSLKRLISLLNEGGELAGEVRLSRRSG [symbol] |
| | | | | | | WSAAVSVRMPVGWKRKRPDSYKLGGASGEIGADPGVIDAVITSTGYSISNISRNP |
| | | | | | | RHIKHVSTPSRTKQERHFAKALSHEDASFERLRYLDEKLKMWQRKASYQYEAA |
| | | | | | | VKANDGKRKGAIMTPKWHEAQTRIRKINAQIANIRAHHRNVIAKILAANYDFVG |
| | | | | | | LESLNTKGMLAKVLPKTDDAGKYIKNGRKAKSGLSRAISAVGFGSLLLLIKQKA |
| | | | | | | GFYGTTVQEINQWFASSKMCSNCGAVKSKAELPLHVREYHCDSCGFVCGRDEN |
| | | | | | | AARNIVNEALRIYHGGNEEETQIDRDIEDVSHQYYKEIVAEIDGVAIDLVEDIV* |
| MG186-2 effector | 1748 | MG186-2 | Protein | Unclassi- | Uncultivated | MATKKKKDNLSQPFYTKQFKLELTPQQKATIEDWMEKLKITWNRGVEALENFR |
| | | effector | | fied | organism | DFIVWNDNIKHPGIACKVANVNGGIFNKRYIRTYYINPKDGTLERENLTKNQKE |
| | | | | | | KYEKIQAPYSLLVSPPTKKIIHRYMKKTKRPTKQGYAFRDRYGEHWIQLTEPTQV |
| | | | | | | EPKDSWGWYEESGLCGYSCPVEFPKNTSLQEDKIEAPVVSAYKASQVRSELGKL |
| | | | | | | LKKETIELMGDADTLSIMQKIRQGKLKDLEAAYDAHRQWLKNPTTLVRNFPKY |
| | | | | | | KRKQDKIRSLQHPSPKGAVSYLGDNFISCSGTGFVRGKNKLGPLKAKSLDERWW |
| | | | | | | TPEGKRPSVSTFRIVRKGNDYFLQLGSNEVKQTRRFTPSNRVIGIDFGLHDFLISD |
| | | | | | | WSGDEREYPQISQVVKPPKELRRQEAKIAKLQQEIHRKREHNLILWLNHPDRKQ |
| | | | | | | DDITQHTRISEENLHKLLQAKTMEEVNDCLGEASVQFFKYNVVPTSQKIKELEA |
| | | | | | | KETDLHEKIKRRRDAFHHKISSFLVDYFDLIIIENGLQEEKMRHRSKIKTNADNTQ |
| | | | | | | YLQNRSKAKSGLTKSLVDSAHGAFMQKLEYKVQIANYQAGYEAKSVESKNAPY |
| | | | | | | TSRDCPCCGARKKEMPVENRWYSCIICGWACDRDVNAALNILFSIDREQWQKYI |
| | | | | | | LSSTANKLLETHHIWNSKRRFYEPSIKQSKSKNRKTKKRSTTVDTAKQLTLNLE* |
| MG187-1 effector | 1749 | MG187-1 | Protein | Unclassi- | Uncultivated | MYKKERAQRKVWRDEALNGIEQAKSEEAARSQDERTQVAKQSFLLHRTREILN |
| | | effector | | fied | organism | LDSLTGNELQTKELWISRKIRGKTPAQNPDAPRIAPRYEAWPVEGEIPEWLRKRF |
| | | | | | | LDEHWLWNALALPLQHTISRIRDQQKQAVAAVYKEAEASGVNTQDEQARTNL |
| | | | | | | APKIRAALKTTDFSPLKQILKHRDLVLEEAHRIRAGKVTDPDAEMGRLGWSKPN |
| | | | | | | WTFAVSVSNLPAWLQRIVEKRIQAAVKAKRTRRQGGASEKWSHGYPKSDPED |
| | | | | | | RSSEGRLDVYFNGDNLQWVSGDMHNGYLDISSPWNPPDHRGPRKGKYRSIREM |
| | | | | | | RKVTIYEPGASAKTRTKKENKNRFSFNVLFHSVPTDALMKEYKLRAQKDAAGK [symbol] |
| | | | | | | LRWFFIPTFELPCSSEPEPRRFAGVDVGWRTSGDEDFKHVHSWSEVRGYRSYEL [symbol] |
| | | | | | | ISPNRWSRRYNHRQAALSADDQFPIEMTPVGLRDFASRRATLLRDLKAKMAEA [symbol] |
| | | | | | | KRQNHLPANWDRIGRRGIARMMTDSKSPESPVLQSLRSEYDTWAKRDEELART [symbol] |
| | | | | | | YRTAWAMVSENLDKQRRQIARDILSGVTDVGIEALDVKQMAQAENQGTTNWE [symbol] |
| | | | | | | RHIENVQDRSRQLTGPAKFLSILVNAARKMGKRVHQIDPCRTSKTCSQCGHVN [symbol] |
| | | | | | | GVEEEFTCSGCARKWNRDENAARNLERLGRECAGADTHPLRCSRDGKSIPFHG [symbol] |
| | | | | | | SRSAVGRASEQSEEASTVLAAEALTHSKQGNYKGGGYF* |
| MG187-2 effector | 1750 | MG187-2 | Protein | Unclassi- | Uncultivated | MKKNERAQRNVWRDETLRAVEQARAEEATRSDDERKTLPKQAFLLQRVREVL |
| | | effector | | fied | organism | NFDLLSGAERETKELWINRKIKGKTPAQNPDAPRLAPRYEAWPVEGEIPAWISK [symbol] |
| | | | | | | FAEERWLWNALALPVPNTISQIRDEQKARVAAVYKEAEANGQDTKDERVRKK [symbol] |
| | | | | | | APKVKAAPKTVDFSPLHRVVKMRDAMLTVAEKLRKGEIPDHDAEMRRLGWTK |
| | | | | | | ENWNFAAAVSKLPSWIQENVKKRLRAASKARSTRKGSGAKEKWWQGYPKVGF |
| | | | | | | EERTGEEQLDIYFNGQNLPWSSSEMHNSYVDISAPCNPPDHRGPRAGKYRRLRE |
| | | | | | | MRTVTIYEPSVSRKMLIRKEHKNRFRLNVLFHAVPDGARMKGYKLCAEHDATR |
| | | | | | | KLRWFFIPTFELPCPTPHEVRTFAGVDAGLRQAGEEEIKAVHVWNEGTNEYKLF |
| | | | | | | PLVTADNRWARRYNARQARLPESERFPIQLTQAGLRDFASRRDILLRDFKRKMG |
| | | | | | | EVLTSGNCLPANWDRVGRRGIARMMTQEKSPEFAALQCLRPEYEAWAKRDGEL |
| | | | | | | ARIYRIAWGMVTENRETQRRRIARDILSSATDIGIEAMEWKKFAEAENEGETNW |
| | | | | | | ERIIIENVIDRNRQIISGPAKFKMVLANLARKMGKRVIIWIAPAFTTKTCSACGQR |
| | | | | | | NEVGRGETFTCTRCGIRWNRDENAARNLARLARECSSGDTHPLKCSRDGKSLPY |
| | | | | | | HGFKERDGKQDGPRLP* |
| MG188-1 effector | 1751 | MG188-1 | Protein | Unclassi- | Uncultivated | MGCAAAGLTEANDKLPIVTSVFDRKEITKRKDAQSGEENPSQSGTTKRDPEWNG |
| | | effector | | fied | organism | TLSADRRALADLGKLAAKKHDNDQLKKKRNQLRWTLIVSLKLEPRGPWYDFIK |
| | | | | | | KAVDKSPFQRTVRKHEKKGQMLNGKNLSYDWPFEEENKTRGLHAKLILSRLPG |
| | | | | | | LRILSVDLGHRHAAACAVWETLTIAQMKKACQEANHDPPTKSDLFLHLARTVA |
| | | | | | | DGKKRTTIYRRIGADNLHDNKEHPAPWARLDRQFLIKLQGEDAPARAASKNEIQ |
| | | | | | | LIDDFAERLGLVPDDRRKHDRSVDELMSQAVLYNAKLGLKRHARRAKIAYALD |
| | | | | | | PNTRSIPGMGGREENFTPGDKDHTKFLTDALLDWRALAADEKWDDKPARELW |
| | | | | | | NQHIAALPDGWRVEDPKPHHETALEPKQRRKDDDALRQRLQPIQPIAKQLIAGD |
| | | | | | | RADMHKAWKERWEKDDGQKAVVPVVPKDQKGPTRTSVSKQATGLHAELRRL |
| | | | | | | TDWIMGRRLQDQTGATTDGWRQNVGGLSLTRIATMKSLYQLHKAFAMRPRPD |
| | | | | | | KPRGAPEKGETNAGVAQSILDAMKRMREQRVKQLASRIVEAALGVGIERCRSA |
| | | | | | | GRDLPRPRERIDNPRFAPCHAVVIENLKHYRPDELQTRWENRQLMNWSSRKVE |
| | | | | | | KYLSEACQLHGLLLREVQAGYTSRQDSRTGAPGVRCADVPVHEFKTAPWWHK |
| | | | | | | QVDAAKKKKEEKNGGSARDKYLLDLIKLDEATWKTQRPLRIPVNGGELFVSASP |
| | | | | | | CHCCKREDRKVAPALQADLNAAANIGLKALLDPDWPGKWWYVPCESTTYKPH |
| | | | | | | AEKTKGSAAIDHGAPLIQAKANVPVTDAPEGKEGKTTSRKAGKKEREIINLWRD |
| | | | | | | PTHAPINSNSSSWRSYKEYRNDVECRVVGILORKLASPSALESAVASGDTPW* |
| MG188-2 effector | 1752 | MG188-2 | Protein | Unclassi- | Uncultivated | MVTRALNFKIVVPRGSDARAYPKALWITHREINHATRYYEELLLSLRQQPISYR [symbol] |
| | | effector | | fied | organism | GSERSAAEALEVAKNLANDATKLNNKNELLGHEDALTLYRHLYEAIVPSSVGE [symbol] |
| | | | | | | GTAQNANMFASPILDPESGGMQSIFEKIKNPPNWIEGVRNGEVASFEAAQSWLG |
| | | | | | | GEQGKERLMASGAPSKWKKAALKKEKDWPTYFVEDYDKKVSEAEGVPTLIRN |
| | | | | | | LRELGLLPLARPYFASRIEGSKAAVTPWDRLAIRLAVAHLLSWESWVSAAATEE |
| | | | | | | QKRVKNVENFKLRSASDTLPTKVADLRKYETQRLEFIISNSVDLGDAPEEFRINQ |
| | | | | | | RMIRGWEDLRARWTRHSDAPDETLIADIAAEQTKKGNRFGDPDLYQWLAKTS [symbol] |
| | | | | | | RGIWSSDEDDPVLYLTSLNEIEMKVERSRETATMTLPDPMLHPRSAQWEPKGG [symbol] |
| | | | | | | NLRNFEINEDASGVLRLSLPLISQNNSGALTEETLEFSLAPSTQMQKPKFSKEGKK |
| | | | | | | IVVEYQQSSGEMARAILGSADLMFNWSHFKNRELEVVENGDVGSVFFKMALDI |
| | | | | | | EPEVSPDIEKIKPGAIYHFQNAAGKKTKHADLVEPGMRVLSVDLGLRSFATCSVF |
| | | | | | | AVQKTGKKIEPGKLFLPISDLGLVAVHERSFSLKLPGEDVGRRGRQWQEDADAE |
| | | | | | | LRALRMALARHRSLRTLASVDQIADRLAQLEQLEARIRDNGWPFEAGLLAELRQ |
| | | | | | | GSPSPEPVWKDLIKTVTRKWRLEFGFPVSEWRKRQRNKDNRKFMGKSIWALDY |
| | | | | | | LGRSRRFLLSWSLIGRGEGDIRRLDRERSGVFAAKLLDHMAGVKADRIKTGADL |
| | | | | | | LVQAARGLGGGTSQKQREACQFILFEDLSRYRMRTDRPRRENSQLAKWSHREIL |
| | | | | | | TETTMQAEIYRIGVVDTGAAFSSRFYALNSAPGMRLSVVRKKDLENPFFIEAVGR |
| | | | | | | ENPHVKTADLKAGSLVFRSGGEDFVTLKRGKICRIQADVNAAQNLQRRLWTRH |
| | | | | | | SEAIRLVTKLVKVDGAEHWVPSTFGERAKGAMGSVGYLVPTGHSSGSCEWKPL |
| | | | | | | KPAQFNKLTGGTEQAIFEDDEELAGLADQFLERSGKVTIFFRDPSGEFLPANLWY |
| | | | | | | PSISFWPIVKQKISAVLGP* |
| MG119-28 targeted | 1753 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-B1 | | | | GrArCrGrArUrGrGrArGrGrUrCrGrUrUrUrCrArGrUrCrUrCrUrGrUrArCrArCrUrCrAr |
| | | | | | | ArArArArArUrUrCrArCrUrUrGrArGrArArArUrCrArArGrUrGrArArUrArUrCrCrArAr |
| | | | | | | CrGrGrGrArCrUrArGrArArArGrGrUrGrArA*mG*mA*mG |
| MG119-28 targeted | 1754 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-B1 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrGrGrGrArCrUrArGrArArArGrGrUr |
| | | | | | | GrArA*mG*mA*mG |
| MG119-28 targeted | 1755 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1-B | | | | UrUrCrArGrUrCrUrCrUrGrUrArCrArCrUrCrArArArArArArUrArUrCrCrArArCrGrGr |
| | | | | | | GrArCrUrArGrArArArGrGrUrGrArA*mG*mA*mG |
| MG119-28 targeted | 1756 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVSI-C3 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrGrGrCrArGrCrUrCrCrCrCrUrArCrCrCrC*mC*mC* [symbol] |
| | | | | | | U |
| MG119-28 targeted | 1757 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-C3 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrGrGrCrArGrCrUrCrCrCrCrUrArCr |
| | | | | | | CrCrC*mC*mC*mU |
| MG119-28 targeted | 1758 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1- | | | | UrUrCrArGrUrCrUrCrUrGrUrArCrArCrUrCrArArArArArArUrArUrCrCrArArCrGrGr |
| | | C3 | | | | CrArGrCrUrCrCrCrCrUrArCrCrCrC*mC*mC*mU |
| MG119-28 targeted | 1759 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-C4 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrGrGrUrCrUrArArCrCrCrCrCrArCrCrUrC*mC*mU* |
| | | | | | | mG |
| MG119-28 targeted | 1760 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVSI-C4 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrGrGrUrCrUrArArCrCrCrCrCrArCr |
| | | | | | | CrUrC*mC*mU*mG |
| MG119-28 targeted | 1761 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1- | | | | UrUrCrArGrUrCrUrCrUrGrUrArCrArCrUrCrArArArArArArUrArUrCrCrArArCrGrGr |
| | | C4 | | | | UrCrUrArArCrCrCrCrCrArCrCrUrC*mC*mU*mG |
| MG119-28 targeted | 1762 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-D | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrGrGrCrArGrArUrUrCrCrUrUrArUrCrUrG*mG*mU* |
| | | | | | | mG |
| MG119-28 targeted | 1763 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-D | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrGrGrCrArGrArUrUrCrCrUrUrArUr |
| | | | | | | CrUrG*mG*mU*mG |
| MG119-28 targeted | 1764 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1-D | | | | UrUrCrArGrUrCrUrCrUrGrUrArCrArCrUrCrArArArArArArUrArUrCrCrArArCrGrGr |
| | | | | | | CrArGrArUrUrCrCrUrUrArUrCrUrG*mG*mU*mG |
| MG119-28 targeted | 1765 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-F1 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrGrGrArArGrUrGrUrArArGrGrArArGrCrU*mG*mC* |
| | | | | | | mA |
| MG119-28 targeted | 1766 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-F1 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrGrGrArArGrUrGrUrArArGrGrArAr |
| | | | | | | GrCrU*mG*mC*mA |
| MG119-28 targeted | 1767 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1-F1 | | | | UrUrCrArGrUrCrUrCrUrGrUrArCrArCrUrCrArArArArArArUrArUrCrCrArArCrGrGr |
| | | | | | | ArArGrUrGrUrArArGrGrArArGrCrU*mG*mC*mA |
| MG119-28 targeted | 1768 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-F4 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrCrCrArGrArArCrCrUrCrUrArArGrGrUrU*mU*mG* |
| | | | | | | mC |
| MG119-28 targeted | 1769 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-F4 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrCrCrArGrArArCrCrUrCrUrArArGr |
| | | | | | | GrUrU*mU*mG*mC |
| MG119-28 targeted | 1770 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrU |
| | | -AAVS1-F4 | | | | UrUrCrArGrUrCrUrCrUrGrUrArCrArCrUrCrArArArArArArUrArUrCrCrArArCrCrCr |
| | | | | | | ArGrArArCrCrUrCrUrArArGrGrUrU*mU*mG*mC |
| MG119-28 targeted | 1771 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-G2 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrUrArGrGrCrCrUrGrCrArUrCrArUrCrArC*mC*mG* |
| | | | | | | mU |
| MG119-28 targeted | 1772 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-G2 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrUrArGrGrCrCrUrGrCrArUrCrArUr |
| | | | | | | CrArC*mC*mG*mU |
| MG119-28 targeted | 1773 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1- | | | | UrUrCrArGrUrCrUrCrUrGrUrArCrArCrUrCrArArArArArArUrArUrCrCrArArCrUrAr |
| | | G2 | | | | GrGrCrCrUrGrCrArUrCrArUrCrArC*mC*mG*mU |
| MG119-28 targeted | 1774 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-H3 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrUrCrUrGrUrCrCrCrCrUrCrCrArCrCrCrC*mA*mC*m |
| | | | | | | A |
| MG119-28 targeted | 1775 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-H3 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrUrCrUrGrUrCrCrCrCrUrCrCrArCr |
| | | | | | | CrCrC*mA*mC*mA |
| MG119-28 targeted | 1776 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1- | | | | UrUrCrArGrUrCrUrCrUrGrUrArCrArCrUrCrArArArArArArUrArUrCrCrArArCrUrCr |
| | | H3 | | | | UrGrUrCrCrCrCrUrCrCrArCrCrCrC*mA*mC*mA |
| MG119-28 targeted | 1777 | 119-28 | Nucleotide | Artificial | | mC*mA*mA*rArCrCrCrCrUrUrCrGrGrGrGrGrArGrGrGrCrGrCrGrUrUrGrGrArGrCr |
| AAVS1 sequence | | sg1_5- | | sequence | | GrCrCrUrUrArGrUrUrUrGrArGrGrUrGrCrArGrArArUrCrArArArArArArArCrUrGrCr |
| | | AAVS1-H5 | | | | GrArCrGrArUrGrGrArGrGrUrCrGrUrUrUrCrArGrUrCrUrCrUrGrUrArCrArCrUrCrAr |
| | | | | | | ArArArArArUrArUrCrCrArArCrCrCrUrGrUrGrArGrArUrArArGrGrCrCrA*mG*mU* |
| | | | | | | mA |
| MG119-28 targeted | 1778 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8- | | sequence | | GrCrArGrArArUrCrArArArArArArArCrUrGrCrGrArCrGrArUrGrGrArGrGrUrCrGrUr |
| | | AAVS1-H5 | | | | UrUrCrArGrUrCrUrCrUrGrUrArCrArCrUrCrArArArArArArUrUrCrArCrUrUrGrArGr |
| | | | | | | ArArArUrCrArArGrUrGrArArUrArUrCrCrArArCrCrCrUrGrUrGrArGrArUrArArGrGr |
| | | | | | | CrCrA*mG*mU*mA |
| MG119-28 targeted | 1779 | 119- | Nucleotide | Artificial | | mC*mA*mG*rGrGrCrGrCrGrUrUrGrGrArGrCrGrCrCrUrUrArGrUrUrUrGrArGrGrUr |
| AAVS1 sequence | | 28_sg1_8.5 | | sequence | | GrCrArGrArArUrCrArArArArArArArCrUrGrCrGrArCrGrArUrGrGrArGrGrUrCrGrUr |
| | | -AAVS1- | | | | UrUrCrArGrUrCrUrCrUrGrUrArCrArCrUrCrArArArArArArUrArUrCrCrArArCrCrCr |
| | | H5 | | | | UrGrUrGrArGrArUrArArGrGrCrCrA*mG*mU*mA |
| DNA Sequence of | 1780 | 119-28 | Nucleotide | Artificial | | GGGACTAGAAAGGTGAAGAG |
| AAVS1 Target Site | | sg1_5- | | sequence | | |
| | | AAVS1-B1 | | | | |
| | | Target Site | | | | |
| DNA Sequence of | 1781 | 119- | Nucleotide | Artificial | | GGGACTAGAAAGGTGAAGAG |
| AAVS1 Target Site | | 28_sg1_8- | | sequence | | |
| | | AAVS1-B1 | | | | |
| | | Target Site | | | | |
| DNA Sequence of | 1782 | 119- | Nucleotide | Artificial | | GGGACTAGAAAGGTGAAGAG |
| AAVS1 Target Site | | 28_sg1_8.5 | | sequence | | |
| | | -AAVS1-B | | | | |
| | | Target Site | | | | |
| DNA Sequence of | 1783 | 119-28 | Nucleotide | Artificial | | GGCAGCTCCCCTACCCCCCT |
| AAVS1 Target Site | | sg1_5- | | sequence | | |
| | | AAVS1-C3 | | | | |
| | | Target Site | | | | |
| DNA Sequence of | 1784 | 119- | Nucleotide | Artificial | | GGCAGCTCCCCTACCCCCCT |
| AAVS1 Target Site | | 28_sg1_8- | | sequence | | |
| | | AAVS1-C3 | | | | |
| | | Target Site | | | | |
| DNA Sequence of | 1785 | 119- | Nucleotide | Artificial | | GGCAGCTCCCCTACCCCCCT |
| AAVS1 Target Site | | 28_sg1_8.5 | | sequence | | |
| | | -AAVS1- | | | | |
| | | C3 Target | | | | |
| | | Site | | | | |
| DNA Sequence of | 1786 | 119-28 | Nucleotide | Artificial | | GGTCTAACCCCCACCTCCTG |
| AAVS1 Target Site | | sg1_5- | | sequence | | |
| | | AAVSI-C4 | | | | |
| | | Target Site | | | | |
| DNA Sequence of | 1787 | 119- | Nucleotide | Artificial | | GGTCTAACCCCCACCTCCTG |
| AAVS1 Target Site | | 28_sg1_8- | | sequence | | |
| | | AAVSI-C4 | | | | |
| | | Target Site | | | | |
| DNA Sequence of | 1788 | 119- | Nucleotide | Artificial | | GGTCTAACCCCCACCTCCTG |
| AAVS1 Target Site | | 28_sg1_8.5 | | sequence | | |
| | | -AAVS1- | | | | |
| | | C4 Target | | | | |
| | | Site | | | | |
| DNA Sequence of | 1789 | 119-28_ | Nucleotide | Artificial | | GGCAGATTCCTTATCTGGTG |
| AAVS1 Target Site | | sg1_5- | | sequence | | |
| | | AAVS1-D | | | | |
| | | Target Site | | | | |
| DNA Sequence of | 1790 | 119- | Nucleotide | Artificial | | GGCAGATTCCTTATCTGGTG |
| AAVS1 Target Site | | 28_sg1_8- | | sequence | | |
| | | AAVS1-D | | | | |
| | | Target Site | | | | |
| DNA Sequence of | 1791 | 119- | Nucleotide | Artificial | | GGCAGATTCCTTATCTGGTG |
| AAVS1 Target Site | | 28_sg1_8.5 | | sequence | | |
| | | -AAVS1-D | | | | |
| | | Target Site | | | | |
| DNA Sequence of | 1792 | 119-28 | Nucleotide | Artificial | | GGAAGTGTAAGGAAGCTGCA |
| AAVS1 Target Site | | sg1_5- | | sequence | | |
| | | AAVS1-F1 | | | | |
| | | Target Site | | | | |
| DNA Sequence of | 1793 | 119- | Nucleotide | Artificial | | GGAAGTGTAAGGAAGCTGCA |
| AAVS1 Target Site | | 28_sg1_8- | | sequence | | |
| | | AAVS1-F1 | | | | |
| | | Target Site | | | | |
| DNA Sequence of | 1794 | 119- | Nucleotide | Artificial | | GGAAGTGTAAGGAAGCTGCA |
| AAVS1 Target Site | | 28_sg1_8.5 | | sequence | | |
| | | -AAVS1-F1 | | | | |
| | | Target Site | | | | |
| DNA Sequence of | 1795 | 119-28_ | Nucleotide | Artificial | | CCAGAACCTCTAAGGTTTGC |
| AAVS1 Target Site | | sg1_5- | | sequence | | |
| | | AAVS1-F4 | | | | |
| | | Target Site | | | | |
| DNA Sequence of | 1796 | 119- | Nucleotide | Artificial | | CCAGAACCTCTAAGGTTTGC |
| AAVS1 Target Site | | 28_sg1_8- | | sequence | | |
| | | AAVS1-F4 | | | | |
| | | Target Site | | | | |
| DNA Sequence of | 1797 | 119- | Nucleotide | Artificial | | CCAGAACCTCTAAGGTTTGC |
| AAVS1 Target Site | | 28_sg1_8.5 | | sequence | | |
| | | -AAVS1-F4 | | | | |
| | | Target Site | | | | |
| DNA Sequence of | 1798 | 119-28 | Nucleotide | Artificial | | TAGGCCTGCATCATCACCGT |
| AAVS1 Target Site | | sg1_5- | | sequence | | |
| | | AAVS1-G2 | | | | |
| | | Target Site | | | | |
| DNA Sequence of | 1799 | 119- | Nucleotide | Artificial | | TAGGCCTGCATCATCACCGT |
| AAVS1 Target Site | | 28_sg1_8- | | sequence | | |
| | | AAVS1-G2 | | | | |
| | | Target Site | | | | |
| DNA Sequence of | 1800 | 119- | Nucleotide | Artificial | | TAGGCCTGCATCATCACCGT |
| AAVS1 Target Site | | 28_sg1_8.5 | | sequence | | |
| | | -AAVS1- | | | | |
| | | G2 Target | | | | |
| | | Site | | | | |
| DNA Sequence of | 1801 | 119-28 | Nucleotide | Artificial | | TCTGTCCCCTCCACCCCACA |
| AAVS1 Target Site | | sg1_5- | | sequence | | |
| | | AAVS1-H3 | | | | |
| | | Target Site | | | | |
| DNA Sequence of | 1802 | 119- | Nucleotide | Artificial | | TCTGTCCCCTOCACCCCACA |
| AAVS1 Target Site | | 28_sg1_8- | | sequence | | |
| | | AAVS1-H3 | | | | |
| | | Target Site | | | | |
| DNA Sequence of | 1803 | 119- | Nucleotide | Artificial | | TCTGTCCCCTCCACCCCACA |
| AAVS1 Target Site | | 28_sg1_8.5 | | sequence | | |
| | | -AAVS1- | | | | |
| | | H3 Target | | | | |
| | | Site | | | | |
| DNA Sequence of | 1804 | 119-28 | Nucleotide | Artificial | | CCTGTGAGATAAGGCCAGTA |
| AAVS1 Target Site | | sg1_5- | | sequence | | |
| | | AAVS1-H5 | | | | |
| | | Target Site | | | | |
| DNA Sequence of | 1805 | 119- | Nucleotide | Artificial | | CCTGTGAGATAAGGCCAGTA |
| AAVS1 Target Site | | 28_sg1_8- | | sequence | | |
| | | AAVS1-H5 | | | | |
| | | Target Site | | | | |
| DNA Sequence of | 1806 | 119- | Nucleotide | Artificial | | CCTGTGAGATAAGGCCAGTA |
| AAVS1 Target Site | | 28_sg1_8.5 | | sequence | | |
| | | -AAVS1- | | | | |
| | | H5 Target | | | | |
| | | Site | | | | |
| MG119-28 targeted | 1807 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_10.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArGrArArArArUrCrCrArArCrGr |
| | | D4 | | | | GrCrArGrArUrUrCrCrUrUrArUrCrUrG*mG*mU*mG |
| MG119-28 targeted | 1808 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_10.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArGrArArArArUrCrCrArArCrCr |
| | | F4 | | | | CrArGrArArCrCrUrCrUrArArGrGrUrU*mU*mG*mC |
| MG119-28 targeted | 1809 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_10.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArGrArArArArUrCrCrArArCrUr |
| | | G2 | | | | ArGrGrCrCrUrGrCrArUrCrArUrCrArC*mC*mG*mU |
| MG119-28 targeted | 1810 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_10.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArGrArArArArUrCrCrArArCrUr |
| | | H3 | | | | CrUrGrUrCrCrCrCrUrCrCrArCrCrCrC*mA*mC*mA |
| MG119-28 targeted | 1811 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_10.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArGrArArArArUrCrCrArArCrCr |
| | | H5 | | | | CrUrGrUrGrArGrArUrArArGrGrCrCrA*mG*mU*mA |
| MG119-28 targeted | 1812 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_11.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArArUrGrArArArArUrArUrCrCr |
| | | D4 | | | | ArArCrGrGrCrArGrArUrUrCrCrUrUrArUrCrUrG*mG*mU*mG |
| MG119-28 targeted | 1813 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_11.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArArUrGrArArArArUrArUrCrCr |
| | | F4 | | | | ArArCrCrCrArGrArArCrCrUrCrUrArArGrGrUrU*mU*mG*mC |
| MG119-28 targeted | 1814 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_11.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArArUrGrArArArArUrArUrCrCr |
| | | G2 | | | | ArArCrUrArGrGrCrCrUrGrCrArUrCrArUrCrArC*mC*mG*mU |
| MG119-28 targeted | 1815 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_11.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrGrArArArArUrArUrCrCr |
| | | H3 | | | | ArArCrUrCrUrGrUrCrCrCrCrUrCrCrArCrCrCrC*mA*mC*mA |
| MG119-28 targeted | 1816 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_11.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrGrArArArArUrArUrCrCr |
| | | H5 | | | | ArArCrCrCrUrGrUrGrArGrArUrArArGrGrCrCrA*mG*U*mA |
| MG119-28 targeted | 1817 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_12.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrGrArArArArArUrArUr |
| | | D4 | | | | CrCrArArCrGrGrCrArGrArUrUrCrCrUrUrArUrCrUrG*mG*mU*mG |
| MG119-28 targeted | 1818 | 119- | Nucleotide | Artificial | | mA*mnG*mG*rGrCrGrCrGrUrUrGrGrArGrCrGrCrCrUrUrArGrUrUrUrGrArGrGrUrGr |
| AAVS1 sequence | | 28_sg1_12.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrGrArArArArArUrArUr |
| | | F4 | | | | CrCrArArCrCrCrArGrArArCrCrUrCrUrArArGrGrUrU*mU*mG*mC |
| MG119-28 targeted | 1819 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_12.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrGrArArArArArUrArUr |
| | | G2 | | | | CrCrArArCrUrArGrGrCrCrUrGrCrArUrCrArUrCrArC*mC*mnG*mU |
| MG119-28 targeted | 1820 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_12.1 | | sequence | | CrArGrArArUrCrArArArArArArArCrUrGrCrGrArCrGrArUrGrGrArGrGrUrCrGrUrUr |
| | | 5-AAVSI- | | | | UrCrArGrUrCrUrCrUrGrUrArCrArCrUrCrArArArArArArUrUrGrArArArArArUrArUr |
| | | H3 | | | | CrCrArArCrUrCrUrGrUrCrCrCrCrUrCrCrArCrCrCrC*mA*mC*mA |
| MG119-28 targeted | 1821 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_12.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrGrArArArArArUrArUr |
| | | H5 | | | | CrCrArArCrCrCrUrGrUrGrArGrArUrArArGrGrCrCrA*mnG*mU*mA |
| MG119-28 targeted | 1822 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_13.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrGrArArArGrArArUr |
| | | D4 | | | | ArUrCrCrArArCrGrGrCrArGrArUrUrCrCrUrUrArUrCrUrG*mG*mU*mG |
| MG119-28 targeted | 1823 | 119- | Nucleotide | Artificial | | mA*mnG*mG*rGrCrGrCrGrUrUrGrGrArGrCrGrCrCrUrUrArGrUrUrUrGrArGrGrUrGr |
| AAVS1 sequence | | 28_sg1_13.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrGrArArArGrArArUr |
| | | F4 | | | | ArUrCrCrArArCrCrCrArGrArArCrCrUrCrUrArArGrGrUrU*mU*mG*mC |
| MG119-28 targeted | 1824 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_13.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrGrArArArGrArArUr |
| | | G2 | | | | ArUrCrCrArArCrUrArGrGrCrCrUrGrCrArUrCrArUrCrArC*mC*mG*mU |
| MG119-28 targeted | 1825 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_13.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrGrArArArGrArArUr |
| | | H3 | | | | ArUrCrCrArArCrUrCrUrGrUrCrCrCrCrUrCrCrArCrCrCrC*mA*mC*mA |
| MG119-28 targeted | 1826 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_13.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrGrArArArGrArArUr |
| | | H5 | | | | ArUrCrCrArArCrCrCrUrGrUrGrArGrArUrArArGrGrCrCrA*mG*mU*mA |
| MG119-28 targeted | 1827 | 119- | Nucleotide | Artificial | | mA*mnG*mG*rGrCrGrCrGrUrUrGrGrArGrCrGrCrCrUrUrArGrUrUrUrGrArGrGrUrGr |
| AAVS1 sequence | | 28_sg1_14.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrArGrArArArUrGrAr |
| | | D4 | | | | ArUrArUrCrCrArArCrGrGrCrArGrArUrUrCrCrUrUrArUrCrUrG*mG*mU*mG |
| MG119-28 targeted | 1828 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_14.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrArGrArArArUrGrAr |
| | | F4 | | | | ArUrArUrCrCrArArCrCrCrArGrArArCrCrUrCrUrArArGrGrUrU*mU*mG*mC |
| MG119-28 targeted | 1829 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_14.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrArGrArArArUrGrAr |
| | | G2 | | | | ArUrArUrCrCrArArCrUrArGrGrCrCrUrGrCrArUrCrArUrCrArC*mC*mG*mU |
| MG119-28 targeted | 1830 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_14.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrArGrArArArUrGrAr |
| | | H3 | | | | ArUrArUrCrCrArArCrUrCrUrGrUrCrCrCrCrUrCrCrArCrCrCrC*mA*mC*mA |
| MG119-28 targeted | 1831 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_14.1 | | sequence | | CrArGrArArUrCrArArArArArArArCrUrGrCrGrArCrGrArUrGrGrArGrGrUrCrGrUrUr |
| | | 5-AAVS1- | | | | UrCrArGrUrCrUrCrUrGrUrArCrArCrUrCrArArArArArArUrUrCrArGrArArArUrGrAr |
| | | H5 | | | | ArUrArUrCrCrArArCrCrCrUrGrUrGrArGrArUrArArGrGrCrCrA*mG*mU*mA |
| MG119-28 targeted | 1832 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_15- | | sequence | | CrArGrArArUrCrArArArArArArArCrUrGrCrGrArCrGrArUrGrGrArGrGrUrCrGrUrUr |
| | | AAVS1-D4 | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrCrArGrArUrUrCrCrUrUrArUrCr |
| | | | | | | UrG*mG*mU*mG |
| MG119-28 targeted | 1833 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_15- | | sequence | | CrArGrArArUrCrArArArArArArArCrUrGrCrGrArCrGrArUrGrGrArGrGrUrCrGrUrUr |
| | | AAVS1-F4 | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrArGrArArCrCrUrCrUrArArGrGr |
| | | | | | | UrU*mU*mG*mC |
| MG119-28 targeted | 1834 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_15- | | sequence | | CrArGrArArUrCrArArArArArArArCrUrGrCrGrArCrGrArUrGrGrArGrGrUrCrGrUrUr |
| | | AAVS1-G2 | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrArGrGrCrCrUrGrCrArUrCrArUrCr |
| | | | | | | ArC*mC*mG*mU |
| MG119-28 targeted | 1835 | 119- | Nucleotide | Artificial | | mA*mnG*mG*rGrCrGrCrGrUrUrGrGrArGrCrGrCrCrUrUrArGrUrUrUrGrArGrGrUrGr |
| AAVS1 sequence | | 28_sg1_15- | | sequence | | CrArGrArArUrCrArArArArArArArCrUrGrCrGrArCrGrArUrGrGrArGrGrUrCrGrUrUr |
| | | AAVS1-H3 | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrUrCrUrGrUrCrCrCrCrUrCrCrArCrCr |
| | | | | | | CrC*mA*mC*mA |
| MG119-28 targeted | 1836 | 119- | Nucleotide | Artificial | | mA*mG*mG*rGrCrGrCrGrUrUrGrGrArGrCrGrCrCrUrUrArGrUrUrUrGrArGrGrUrGr |
| AAVS1 sequence | | 28_sg1_15- | | sequence | | CrArGrArArUrCrArArArArArArArCrUrGrCrGrArCrGrArUrGrGrArGrGrUrCrGrUrUr |
| | | AAVS1-H5 | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrCrCrUrGrUrGrArGrArUrArArGrGrCr |
| | | | | | | CrA*mG*mU*mA |
| MG191 PAM | | MG191-51 | Nucleotide | | | TTR |
| | | PAM | | | | |
| MG191 PAM | | MG191-53 | Nucleotide | | | TtR |
| | | PAM | | | | |
| MG191 PAM | | MG191-1 | Nucleotide | | | R |
| | | PAM | | | | |
| MG191 PAM | | MG191-2 | Nucleotide | | | R |
| | | PAM | | | | |
| MG191 PAM | | MG191-5 | Nucleotide | | | TRR |
| | | PAM | | | | |
| MG191 ancestral | 1842 | MG191-32 | Protein | | | MANHKKKEENEIVKTFTFKVKNSNNETCSIMKDAIEEYQNYYNKVSDWINDNL |
| effector | | ancestral | | | | TSMTIGDLAQYIPEKSKDTAYYKYALSDEWKDKPLYKIFTKGFNSNNADNALY [symbol] |
| | | effector | | | | AIKKVNVDNYNGNILGFSETYYRRNGYVKNVISNYRTKMSTLKPKIKRKKIDEN |
| | | | | | | STEETLMEQTIYEIQKHGLKSTKDWEDQIEYLKNKENPNQNYIERLKTLYDYYK |
| | | | | | | NHKDTINEKMEELSVEQLKEFGGCRRKNDKTSMTIQIQNNSPFKIRKEGSTGYIL |
| | | | | | | NLPLNKKNFNIDLWGNRQVIKNNKELVDIVNKHGDSITFKIKNNELYVTITCDVD |
| | | | | | | FEKKESEIKKVVGVDVNIKHMLLATSIKDNGNLKGYINIYKELLNDKDFVKLLN |
| | | | | | | KEELKYFKDLSKYVTFCPIEFDFLFSRVMNQKDLNDKYTKKEKAFSNVLNKLQK |
| | | | | | | KFKDNNDTKEANYINNVKKLRAQYKAYFILKQKYYEKQKEYDLKMGFTDESES |
| | | | | | | KEDKRRFENPFTNTETAKEILSKMNNVSQDIIGCRDNIITYAYNVFENNGYDTIAL |
| | | | | | | ENLESSQFEKHKRLPTPKSLLNYHKLQGKTIDEVEKNEKVSKIKKDYYNFEFDD |
| | | | | | | NKITDVKYSEKGEMKKKKNLFYNLVIKAIHFADIKDKFIQLSNNGKVQVALVPS |
| | | | | | | QFTSQMDSITIIKLYMKENKNKGKLVLVDKRKVRKTQERIIINGLNADYNAACNI |
| | | | | | | AYIVENEEWRNTFCKRTPKKKNTYNTPVFDTKKKSQKNIVNQLKKLGMTKILEV |
| | | | | | | DEC |
| MG191 ancestral | 1843 | MG191-33 | Protein | | | MAKKKKKTENEIVKSFKFKVKNSNQETCDITKDVIEEYKNYYNKVSNWINNNL |
| effector | | ancestral | | | | TSMTIGDLAQYLPEKSKTTGYIKYALSDEWKDKPLYYIFTKKYNSNNANNLLYE |
| | | effector | | | | AIKNLNVDNYNGNILGFSETYYRKNGYVKRVISNYRTKISTLNVKIKRKKVDEN |
| | | | | | | STVETLEEQTMYEINKLGLKTKKDWEEHIEYLKNQENPNQNTIDRFKILYDYFK |
| | | | | | | NNKDTINEKMEELSIEQLKEFGGCKMKNNTTSMTIQIQNNSPFKIRKENSLGYILN |
| | | | | | | IPLNKKNFNIELWGHRQVIKGNKELVDIVNKHGNNITFKIKNNELYVIITSDTEFE |
| | | | | | | KKETNFEKIVGVDVNIKHALMVTSEKDNGNLKGYINLYKEVLNNDEFTKLLNK |
| | | | | | | EELKNFKDLSKYVTFCPIEYDFLFSRIDKQKDFNDKYTKKEQAFSNVLYDLQKKF |
| | | | | | | KNENNTKEYNYISCVNKLRAKYKAYFVLKQKYYEKQKEYDIKMGFVDESESKE |
| | | | | | | DKRRFENPFTNTEVAKEILSKMNNVQQDINGCRKNIITYAYKVFENNGYDTIALE |
| | | | | | | NLENSNFEKHKPLPSIKSLLNYHKLQGKTIDEVEQNEKVSNIEKDYYIFQFNENKI |
| | | | | | | IDAKYSQKGYYKVKKALFFNQMIKTLHFASIKDEFIQLSNNGKVQVALVPSEYTS |
| | | | | | | QMDSITHKLYMIKNKDKGKLVKVDKRKVRKTQERHINGLNADFNAACNIKYIV |
| | | | | | | ENEEWRNVFCKRTPKKKNTYNTPVLDATKKGQSNIINQLKKLGATKLLEVEEA |
| MG191 ancestral | 1844 | MG191-34 | Protein | | | MAKNNKKTENEIVKSFKFKVKNSNQETCDITKDVIEEYKEYYNRVSNWINNNLT |
| effector | | ancestral | | | | SMTIGDIAQYLKEKTKTTGYIKVALSDEWKDKPMYYLFTKKYNSNHANNLLYE [symbol] |
| | | effector | | | | FIKNNNVDNYNGNILGLSETYYRKNGYVKLVISNYRTKINTLNCKIKRKKVDVN |
| | | | | | | STVEDIEKQTMYEIIKLGLKKKKDWEEHIEYLKNQENPNQNSINRFKILYDYFNN |
| | | | | | | NEDTINEKMELLSIEQLKEFGGCKMKNNTTSMTIQIQNNSPFKIEKENSLGYILN [symbol] |
| | | | | | | LNKKNFNIELWGHRQIKKGNKELVDIINKHGNNIVFKIKNNELYVILSSDTEFEK |
| | | | | | | KETNFEKIVGVDVNIKHALLVTSEKDNGNLKGYINLYKEVLNNDEFTSLLNKEE |
| | | | | | | LKNFKDLSKYVTFCPIEYDFLFSRIDKQKDFNDKYTKKEQVFSKILYDLQKKFK [symbol] |
| | | | | | | ENNTKEYNYISCVNKLRAKYIAYFILKQKYYEKQKEYDIKMGFVDDSESKEDK [symbol] |
| | | | | | | RFENPFTNTEVAKEILSKMNNVQQDINGCRKNIITYVYKVFENNGYDTIALENL [symbol] |
| | | | | | | NSNFEKHKPLPSIKSLLNYHKLQNKTIEEVKQNDKVSEIEKDYYVFTFNENKIID [symbol] |
| | | | | | | KYSQKGYYKVKKALFFNLMIKTLHFASIKDEFIQLSNNGKTQIALVPSEYTSQMD |
| | | | | | | SITHKLYMIKNKDKGKLVKVDKRKVRKTQERHINGLNADFNAACNIKYIVENEE |
| | | | | | | WRNVFCKRSPKKKNTYNTPVLDATKKGQYNIINQLKKLGATKLLEMEKA |
| MG191 ancestral | 1845 | MG191-35 | Protein | | | MANHKKKEENEITKTFVFKAKMSNNDTMSLWKPAIEEYQNYYNKVSDWICDN |
| effector | | ancestral | | | | LTSMKIGDLAQYIDEKSKDTAYYKYVLSDEYKDKPLYKIFTKGFNSNNADNALY |
| | | effector | | | | EAIKKINVDNYNGNILGLSETYYRRNGYVKNVISNYRTKMSTLKPNIKRKKIDEN |
| | | | | | | STEETLMEQTIYEIQKHGLKSTKDFEDLIEYLKNKENPNQNYIERLKTLYDYYKN |
| | | | | | | HKDEINEKMEEMAVEQLKKFGGCRRKNDKTSMTIQIQNNSPFKIRKDGSTSYILH |
| | | | | | | LPLNKKNYNINLWGNRQVVKNNNDLVDIVNKHGDSITFKIKNGELYVTLTCDV |
| | | | | | | DFEKKESEIKNVVGVDVNIKHMLMATSIKDNGNVKGYINIYKELLNDEDFVKLL |
| | | | | | | NKEELKYFKDLSKYVTFCPIEFDFLFSRVVNQKDLNDKLTKREKAFSNVLDKLQ |
| | | | | | | KKYKDNNDTKIANYINYVKKLRAQYKAYFILKQKYYEKQKEYDLKMGFTDESE |
| | | | | | | SKEDKRRFENPFINTETAKEILSKMNNVEQDIIGCRDNIITYAYNVFENNGYDTIA |
| | | | | | | LENLESSQFEKHKRLPTPKSLLNYHKLEGKTIDEVEKNEKKSKINKDYYNFEFDD |
| | | | | | | NKITDIKYSEEGERKKKKNLFYNLVIKAIHFADIKDKFIQLSNNNKVQVVLVPSA |
| | | | | | | FTSQMDSKTHKLYVTETKNKKKLVLVDKRKVRKQQERHINGLNADYNAACNI |
| | | | | | | AYIVENEELRNTMCKRTPKKKNTYNTPVFNTKKKSQKNIVNTLKKMGMTKILE |
| | | | | | | VDEAGEFAKN |
| MG191 ancestral | 1846 | MG191-36 | Protein | | | MANHKKKEENTITKSFVFKAKVSKNDIMSLWKPAIEEYTNYYNKVSDWICDNL |
| effector | | ancestral | | | | TSMKIGDLAQYIDEKSKDSAYYKYVIDDEYKDLPLYKIFTKGFSSQNADNALYE |
| | | cffcctor | | | | AIKKINPENYNGNILGLGETYYRRNGYVKNVISNYRTKMSTLKPNIKYKKFDENS |
| | | | | | | TEETLMEQTIYEVTKHGIESTKDFENLIEYLKNKENPNTNKIERLETLYDYYKNH |
| | | | | | | KDEINEKIEEMAVEQLKKFGGCRRKSDNTSMTIHMQNNSPSKIRKDGLTSYILHL |
| | | | | | | VFNKKPYDINLLGNRQVVKVNNDI,VDICNKHGDSITFKIKNGELFVTLTSEVDFD |
| | | | | | | KKESEIKNVVGVDVNIKHMMLATSIVDNGNVKGYINIYKELLNDEDFVSTLNNE |
| | | | | | | ELEYYKEMSKYVNFGLLELDSLFSRVTNQKDLDDKLLKREIAISNVLDKLAKKY |
| | | | | | | RDNKDTKIANYINYTKMLRAKYKAYFILKOKYYEKQKEYDLKMGFTDESESKE |
| | | | | | | DKRRFENPFINTETAKEILTKLNNVEQDIIGCRDNIITYAFNVFKNNGYDTIALEY |
| | | | | | | LDSSQFEKHKRLPTPKSLLNYHKFEGKTEDEVEKSEKKNKFNKDYYNFEFDDN [symbol] |
| | | | | | | ITDISYSQEGLREKKKNNFYNLIIKAIHFADIKDKFVQLSNNNKMNVVFVPSAFT [symbol] |
| | | | | | | QMDSKTHKLYVTETKNKKKLVLVDKRKVRKQQERHINGLNADYNAACNIKYI |
| | | | | | | AENEELRNTMCKRTKKDKNTYNTPFYNIKKKFKKNISNTLKKMGMVKICDIDE |
| | | | | | | AGEFAKN |
| MG191 ancestral | 1847 | MG191-37 | Protein | | | MANHKKKEENTITKSFVFKANVSKNDIMSLWKPAIEEYTNYYNKVSDWICDNL |
| effector | | ancestral | | | | TSMKIGDLAQYIDEKSKDSAYYKFVIDDEYKDLPLYKIFTKGFQTQNADNALYE |
| | | effector | | | | AIKKINPEGYNGNILGLGETYYRRNGYVKNVISNYRTKMSTLKPNIKYKKFDEN |
| | | | | | | TEEVLMEQTIYEVVKHGIESKEDFENLIEYFKNRETPNTNKIKRLETLYDYYKNH |
| | | | | | | KDEINEKIEEMAVEQLKSFNGCRRKGDNKSMTIHMQNNSPSKLRKDGLTSYILH |
| | | | | | | LVFNKKPYDINLLGNRQVVKVNNDLVDICNKHGDSITFQIKNGELFVTLTSEVDF |
| | | | | | | DKKESEIKNVVGVDVNIKHMMLATSIVDNGNVKGYINIYKELLNDKDFVSTLNN |
| | | | | | | EELEYYKEMSKYVNFGLLEVDSLFSRVSNQMDLDDKLLKREIAISNVLDNLAKK |
| | | | | | | YRDSKDCKIASYIDYTKMLRAKYKAYFILKQKYYEKQKEYDLKMGFTDESNSK |
| | | | | | | EDKRRFENPFINTETAKELLTKLNNVESDIIGCRDNIINYAFNVFKNNGYDTIALE |
| | | | | | | YLDSSQFEKHKRIPTPKSLLNYHKFEGKTIDEVEKSMKKNKFNKDYYNFEFDDN |
| | | | | | | KITDISYSQRGLREIKKKNFYNLIIKAIHFADIKDKFVQLSNNNKMNVVFVPSAFT |
| | | | | | | SQMDSKTHKLYVTETKNKKKLVLVDKRKVRKQQERHINGLNADFNAACNIKYI |
| | | | | | | AENEELRNTMCKRKKKDKNTYGTPFYNIKDTFKKNISNTLKKMGMVKICDIDE |
| | | | | | | AGEFAKN |
| MG191 ancestral | 1848 | MG191-38 | Protein | | | MANHKTKETGTITKSFKFKANVSKNEIMSLWKPAIEEYTNYYNKVSDWICDNLT |
| effector | | ancestral | | | | SMKIGDLAQYIDEKSKDSAYYKFVVDDEYKDLPLYKIFTKGFQTQNVDNALFAA |
| | | effector | | | | IKKINPEGYNGNILGLGETYYRRNGYVKNVISNYRTKMSTLKPNIKYKKFDENST |
| | | | | | | EEVLMEQTIYEVVKHGIESKEDFENLIENFKNRETPNTNKIKRLETLYSFFKNHKD |
| | | | | | | EIIEKMEEMAVEQLKSFNGCRRKGDNVSMTIHMQNNSPSKLRKNGLTSFILHIVF |
| | | | | | | NKKPYDINLLGNRQVVKVNNDLVDICNNHGDYITFQIKNGEMFVILTSEVDFDK |
| | | | | | | KESEIKNVVGVDVNIKHMMLATSIVDNGNVKGYINIYKELLNDEDFVSTCNNEE |
| | | | | | | LEYYKEMSKSVNFGLLECDSLFSRVSNQMDLDDKLLKREIAISNVLDRLAKKYR |
| | | | | | | DSKDCKIASYIDYTKMLRAKYKAYFILKQKYYEKQKEYDLKMGYTDESNSKED |
| | | | | | | KRRFENPFINTETAKELLTKLNNIESDLIGCRDNIINYAFNVFKNNGYDTIALEYL |
| | | | | | | DSSQFDKHKRIPTPKSLLNYHKFEGKTIDEVEKSMVKNKFNKDNYNFEFDDNKI |
| | | | | | | TDISYSQRGLREIKKKNFYNLIIKAIHFADIKDKFVQLSNNNKMNIVLVPSAFSSQ |
| | | | | | | MDSKTHKLYVTETKNKKKLVLVDKRKVRKQQERHINGLNADFNAACNLKYIA |
| | | | | | | ENEELRNTVCKRKKKDKNTYGTPFYNIKDAFKKNVSNTLKKMGMVKICDFDE |
| | | | | | | GSFAKR |
| MG191 ancestral | 1849 | MG191-39 | Protein | | | MAHHKKKEENTITKTFVFKAKVSKNDIMSLWKPAIEEYTNYYNKLSDWICDNL |
| effector | | ancestral | | | | TSMKIGDLAQYIDEKSKDSAYYKYVIDEEKKDLPLYKIFTKGFSSQNADNALYE |
| | | effector | | | | AIKKINPENYNGNILGLGETNYRRNGYVKNVISNYRTKMSTLKPNIKYKKFDEN |
| | | | | | | NTEETLMEQTIYEVTKHGIESTKDFENLIEYLKNKEEPNTEKIERLETLYDYYKN |
| | | | | | | HKDEINEYIEEMAVEQLKKFGGCRRKSDNTSMTIHMQNNSPSKIKKDGLTSYTL |
| | | | | | | HLVFNKKPYTINLLGNRQVVKVANDLVDICNKHGDSITFKIKNGELFVTLTSEVI |
| | | | | | | FEKKESEIKNVVGVDVNIKHMMLATSIVDNGNVKGYINIYKELLNDEDFVSTLN |
| | | | | | | NEELEYYKEMSKYVNFGILELDSLFSRVTNQQKLDNKLIKRELAIONVFDKLAK |
| | | | | | | KYRDNKDTKIANYINYTKMLRAKYKAYFILKEKYYEKQKEYDLKMGFTDESES |
| | | | | | | KEDKRRFENPFINTETAKEILTKLNNVEQDIIGCRDNIVTYAFNVFKNNGYDTIAL |
| | | | | | | EYLDSSQFEKRKRMPTPKSLLNYHKFEGKTEDEVEKSMKKNKFSKDYYNFEED |
| | | | | | | DNKITDISYSNEGIRKQKKNNFYNLIIKAIHFADIKDKFVQLSNNNKMNVVFVPS |
| | | | | | | AFTSQMDSKTHKLYVVETKNKKKLVLVNKRKVRKQQERHINGLNADYNAACN |
| | | | | | | IKYIAENEELRNTMTKRTKKDKNTYNTPAYNIKKKFKKNLSNTLKKMGMVKIC |
| | | | | | | EIDENGEFAKN |
| MG191 ancestral | 1850 | MG191-40 | Protein | | | MAHKKKKGENTITKTFVFKAKCDKNDIISLWKPAAEEYCNYYNKLSKWICDNL |
| effector | | ancestral | | | | TSMKIGDLAQYIDEKSKDSAYYKYVIDEEKKDLPLYKIFTKGFSSQYADNALYC |
| | | effector | | | | AIKKINPENYNGNILGLGESNYRRNGYVKNVISNFRTKMSTLKANIKYKKFDPN |
| | | | | | | NDEETLMEQTIYEVDKHGIESTKDFEDLIEYLKTREETNNDKIERLECLMDYYKN |
| | | | | | | NKDEINEYIEEMAVEDLKKFGGCQRKSNNTSMTIHMQNNSPSKIKKDGLTSYNL |
| | | | | | | QLVFNKKPYTINLLGNRQVVKVVNDLIDICDKHGDSITFKIKNGELFVHLTSPVD |
| | | | | | | FEKKEHEIKNVVGVDVNIKHMMLATSIVDDGNVKGYINLYKELLNDEDFVSTLN |
| | | | | | | NSELEMYKQMSENVNFGILETDSLFERVVNQQKLDNKLIKRELAIQNVFDNLAK |
| | | | | | | TTRDSKDTKIANYINYTKMLRAKYKAYFILKEKYYEKQKEYDLKMGFTDESESK |
| | | | | | | EDKRRFENPFINTETAKEILTKLNNVEQDIIGCRDNIVTYAFNVFKNNGYDTIALE |
| | | | | | | YLDSSQFDKRRRMPTPKSLLKYHKFEGKTEDEVEKMMKENKFSKSYYNFEYDN |
| | | | | | | NVITDIEYSNEGIRKQKKNNFMNLIIKAIHFADIKDKFVQLSNNNKMNVVFVPSA |
| | | | | | | FTSQMDSKTHSLYYVETKEKKKYVLANKKTVRTQQERHINGLNADYNSACNIK |
| | | | | | | YIAENEELRNKMTKRTKKGKNMYNTPAYNIKKSFKKNLSNTLKKMGNVKYGKI |
| | | | | | | DENGEFVEN |
| MG191 ancestral | 1851 | MG191-41 | Protein | | | MAHKKNNGENTITKTFIFKAKCEKNDIISLWKPAAEEYCNYYNKLSKWIGDNLT |
| effector | | ancestral | | | | SMKIGDLAQYIDEKSKNSAYYKAVTDESKKDLPLYKIFQKGFSSQYADNALYCA |
| | | effector | | | | IKKINPENYNGNILGIGESDYRRNGYVQSVISNFRTKMSSLKANIKWKKFDPNND |
| | | | | | | EETLKTQTIYEVDKYGIESTKDFEDLIEVLKTREETPNDKIERLECLMDYYKNNE |
| | | | | | | DTIKDYIETMAIEDLQKFGGCQRKSLNLTLTIHKQNNSPSKMEKDGNTSFNLQLV |
| | | | | | | FNKKPYTINLLGNRQVVKFVNDLIDITEKHGDWITFNIKNGELFVHLTSPVDFEK |
| | | | | | | KEHEIKNAVGVDVNIKHMMLATSIVDDGNVKGYINLYKELLNDKDFVSTENNS |
| | | | | | | ELEMYRQMSENVNFGILETDSLFERVVNQQKLDNKLIRRELAIQNVFDNITKTTE |
| | | | | | | DSKDQRIVDYINYVKMLRAKYKAYFILKEKYYEKQKEYDLMMGFTDESESKEL |
| | | | | | | KRRMEFPFINTETAKEILTKLNNVEQDIIGCRDNIVTYAFNVFKNNGYDTISLEYI |
| | | | | | | DSSQFDKRRRMPTPKSLLKYHKFEGKTEDEVEQMMKEKKFSKSYYNFKYDND [symbol] |
| | | | | | | VSDIEYSNEGIWKQKKLNFMNLIIKAIHFADIKDKFVQLCNNNDMNVVFCPSAF [symbol] |
| | | | | | | SQMDSKTHSLYYVEKKEKKKYVLANKKTVRTQQERHINGLNADYNSACNLKY |
| | | | | | | AENEELRNKMTKRTKKQKTMYNTPAYNIKKSFKKNLSNTFRKLGNVKYGKIN [symbol] |
| | | | | | | DGQFVEN |
| MG191 ancestral | 1852 | MG191-42 | Protein | | | MAHKTNNGENTINKTFIFKAKCEKNDIISLWKPAAEEYCNYYNKLSKWIGDNLIS |
| effector | | ancestral | | | | MKIGDLAQYIDEKSQNSAYYLAVTDESKKDLPLYKIFQKGFSSLCADNALYCAI |
| | | effector | | | | KKINPENYNGNILGIGESDYRRNGYVQSVISNFRTKMSSLKATVKWKKFDVNND |
| | | | | | | EETLKTQTICDVDKYGIESVKDFKELIEVLKTREETPNDKIARLECLCDYYSKNE |
| | | | | | | KTIKNEIETMAIADLQKFGGCQRKSLNLTLTIHKQNNSPSKIEKVGNTSFNLQLVF |
| | | | | | | NKKPYTINLLGNRQVVKFVNDLIDITEKHGDWITFNIKNGELFVHLTSPVEFEKK |
| | | | | | | VHEIKNAVGVDVNIKHMMLATSIVDDGNVKGYINLYKELLNDNDFVSTENDSE |
| | | | | | | LEMYRKMSKNVNFGILEVDSLFERVVNQQKLNNKLIRRERAMQKVFDNIVKTT |
| | | | | | | RDSKDQRIIDYVNYVKMLRAKYKAYFILKEKYYEKQKEYDLMMGFTDESESKE |
| | | | | | | DKRRKEFPFINTETAKEILSKLNNVEQDIIGCRDNIVTYAFNVFKNNGYDTISVEY |
| | | | | | | LDSSQFDKRRRMPTPKSLLEYHKFKGKTEDEVEQLMKEKKFSKAYYNFKYDND |
| | | | | | | VVSDIEYSKEGEWRQKKLNFMNLIIKAIHFADIKDKFVQLCNNNDMNVVFCPSA |
| | | | | | | FTSQMDSKTHSLYYVEKKEKKKYVLANKKSVRTQQETHINGLNADYNAACNL |
| | | | | | | KYIAENEELLEKMTKRTKKGKTMYNTPAYNIKKKFKKNLSNTFRKLGNVKYGK |
| | | | | | | INNDGQFVED |
| MG191 ancestral | 1853 | MG191-43 | Protein | | | MANHKKKEENEIVKTFTFKVKNSNGETCDIMKDAIKEYQNYYNKVSDWINDNL |
| effector | | ancestral | | | | TSMTIGDLAQYIPEKSKDTGYYKYALSDEWKDKPLYKIFTKGFNSNNADNALYE |
| | | effector | | | | AIKKVNVDNYNGNILGFSETYYRRNGYVKNVISNYRTKMSTLKPKIKRKNIDEN |
| | | | | | | STEDTLMEQTIYEIQKNGLKSTKDWEDQIEYLKNKEDSNQNYIERLQTLYDYYK |
| | | | | | | NHKDTVNEKMEELSVESLKKFGGCRRKNDKKSMTIQIQNNSPFKIRKEGRNGYI |
| | | | | | | LNFKKNFNIDLWGNRQVIKNGEELVDIVNKHGNSITFKIKNNEMYVNITCDVPFE |
| | | | | | | KKESEIKKVVGVDVNIKHMLLATSIKDNGNLKGYVNIYKELLNDKDFVKVCNK |
| | | | | | | EELKYFKDLSKYVTFCPIEFDFLFSRVINQKDLNDKYTKTEEAFSNVLNKLQKKF |
| | | | | | | KDSGDNKERIYIENVMKLRAQLKAYFILKNAYYEQQSEYDLGESKERRFENPFR |
| | | | | | | NTEKAKEILSKMNNISQKIIGCRDNIITYAYNVFENNGYDTIALENLESSQFEKHK |
| | | | | | | RLPTPKSLLNYHKLQGKTIDEVEKNEKVSKIKKGYYNFEFDDNKITDVKYSEKG |
| | | | | | | EMQKKKNLFYNLVIKAIHFADIKDKFIQLSNNGKVSVALVPSQFTSQMDSITHK [symbol] |
| | | | | | | YMKKNKKNGKLVLVDKRKVRKTQERHINGLNADYNAACNIAYIVENEEWRNT |
| | | | | | | FCKQTPTKKNTYNTPLFDTKIKSQKNIVNKLKKLGMTKILEVDEC |
| MG191 ancestral | 1854 | MG191-44 | Protein | | | MAHHKKKEENEIVKTFSFKVKNSNGVTMDVLKDAIKEYQNYYNKVSDWINDN |
| effector | | ancestral | | | | LTMTIGDLAQYIPEKSKDTGYYKYLISDEWKDKPLYKIFTKGFNSNNADNALYE |
| | | effector | | | | AIKTVNVDNYTGNILGFSETYYRRNGYVKNVISNYRTKLSKLKPKIKRKNISENS |
| | | | | | | TEDTLMEQCIYEIEKNGLKSVKDWEDQIEYLKEKEDSNQNYLERLQTLYDYYKI |
| | | | | | | HKDTVNEKMEEMSVESLKKFGGCRRKNDKKSMTIQLQNNSPFKITKEGRNGYI [symbol] |
| | | | | | | NFKKNFNIDLWGNRQVIKNGEELVDIVNKHGNSITFKIKNDEMYVNITCSVPFEK [symbol] |
| | | | | | | KESEINKVVGVDVNIKHMLLATSIKDNGNLKGYVNIYKELLNDKDFVKVCNKE |
| | | | | | | ELKYFKDLSKYVTFCPIEFDLLFSRVMNQKDLNDKYTKTEEAFSNVLNKLQKKF |
| | | | | | | KDSGDNKERIYIENVMKLRAQLKAYFILKNAYYEQQSEYDLGESKERRFENPFS |
| | | | | | | NTEKGKEILSKMNNISQKIIGCRNNIITYAYNVFENNGYDTIALEKLESSQFEKKR |
| | | | | | | LPTPKSLLNYHKLQGKTIDEVEKNEVFSKIKKGYYNFEFDDDKITDVKYSEKGE |
| | | | | | | MQKIKNDFYNLVIKAIHFADIKDYFITLSNNGKVSVALVPSQFTSQMDSIEHKLY |
| | | | | | | MKKNKKNGKLVLVDKRKVRKTQERHINGLNADYNAACNIAYIVENEEWRNTF |
| | | | | | | MKQTPTKKNTYNTPLFDTKIKSQKNIVNKLKKLGMTKILEVDEC |
| MG191 ancestral | 1855 | MG191-45 | Protein | | | MAKHKKKEENEIVKTFSFKVKNSNGITMDTLKDAIKEYQNYYNKCSDWINDNL |
| effector | | ancestral | | | | TMTIGDLYQYIPEKSKDTGYAKFLISDEWKDKPLYKIFTKGFNSNNADNALYEAI |
| | | effector | | | | KTVNVDNYTGNILGFSETYYRRNGYIKNVISNYRTKLSKLNTKIKRKNISENSDE |
| | | | | | | DTLMEQCIYEIEKNGLESVKDWEDQIEYLKSKEDSNQNYLERMQTLYDFYKNH |
| | | | | | | KDEVNEKMEEMSVESLAKFGGCRRKNDKKSMSIQLQSNSPFKITQIGRNSYNIKF |
| | | | | | | KKNFNIDVWGRRDVIKNGELLVDIINKHGNSIVFKIKNDEMYVNITCSVPFNKKE |
| | | | | | | STINKVVGVDVNIKHMLLATSITDNGNLKGYVNIYKELINDKDFKKVCNKEEMK |
| | | | | | | YFKDLSKYVTFCPIEFDLLFSRVIKQKDINDKNTKMEEAFSNVLNKLKKKFKESG |
| | | | | | | DNKERIYIENVMKLRSQLKAYCILKNAYYKQQSEYDLGESKEYIFEHPFSNTEKG |
| | | | | | | KEILSKMNNISKKIIGCRNNIIQYAYNVFEKNGYDMIALEKLTSSQFEKKRMPTPK |
| | | | | | | SLLNYHKLLGKTQEEIEKNEVYSVIKKGYYTFEFDDDKITDAKYSEKGEMQKIK |
| | | | | | | NDFYNLMIKAIHFADIKDYFITLSNNGTVSVALVPSQFTSQMDSIEHKLYFKNNK |
| | | | | | | KNGKLVLVDKRKVRKTQERHINGLNADYNAANNIAYIVENEEWRNTFMKQTR |
| | | | | | | TDKSTYNTPSYDTKIKSQKNIVNKLKKLGMTKILEADEC |
| MG191 ancestral | 1856 | MG191-46 | Protein | | | MAKHKKTESNEIVKTFSFKVKNANGITMDTLKDAIKEYQNYYNKCSDWICDNL |
| effector | | ancestral | | | | TMTIGDLYQYIPEKSKDTGYAKFLISDEWKDKPLYKIFSKGYSSNNRDNALYEAI |
| | | effector | | | | NTVNVENYTGNILGFSETYYRRNGYIKNVISNYATKI,SKINTKIKRKNISENSDE |
| | | | | | | DTLMEQTIYEMEKNGLESVKDWEDQIEYLESKTDSNQNYLERIKTLYDFYKDHK |
| | | | | | | DEVNSKMEEMSVESLVKFGGCRRKDSKKSMSIQLASNSPFKITQIGGNSFNIKFS |
| | | | | | | KNFNIDVWGRRDVIKNGELLVDIINKHGNSIVFKIKNDEMYVDITCSVPFNKKES |
| | | | | | | TINKVVGVDVNIKHMLLATSITDNGNLKGYVNIYKEVINDPDFKKVCNKEEMK |
| | | | | | | YFKDISKYVTFCPIEFDLLFSRVCKQKDINDKNSKMEEAFSNVLNKLKQKFIESG |
| | | | | | | DNKNRIYIENVMKLRSQLKAYCILKNAYYKQQSEYDLGKSEEYIQEHPFSNTEK |
| | | | | | | GKEILSKMDNISQKIIGCRNNIIQYAYNIFEKNGYDMISLEKLTSSQFEKKSMPTV |
| | | | | | | KSLLNYHKILGKTQEEIEKNDVYSVIKKGYYTFEFDDDKVTDAKYSEKGELQKI |
| | | | | | | KNDFFNLMIKAIHFADIKDYFITLSNNGTVGVSLVPSQFTSQMDSIEHKIYFVNNI |
| | | | | | | KNGKLTLADKRKVRKTQERHINGLNADYNAANNIAYIVEDETWRNTFMKQSR |
| | | | | | | DKSLYNTPSYDTSIKSQGNVVSKLKKLGMVKILEADEV |
| MG191 ancestral | 1857 | MG191-47 | Protein | | | MAKHKKTESNEIVKTFSFKVKNANGISMDTLKDAIKEYQNYYNLCSDWICDNLT |
| effector | | ancestral | | | | MTIGDLYQYIPEKKKDTGYAKVLISDEWKDKPLYKIFSKGYSSNNRDNALYEAI |
| | | effector | | | | NTVNVENYTGNILGFSDTYYRRNGYIANVISNYATKLSKLNTGIKRKNISENSDE |
| | | | | | | DTLMEQVIYEMEHNGWESVKDWENQIEYLESKTDSNPNYLERMKTLYDFYKSH |
| | | | | | | KDEVNSKMEEMSVENLVKFGGCRRKDSKKSMYIMGASNTPFTITQIGGNSFNIK |
| | | | | | | FSKNLNIDVYGRRDVIKNGELLVDIINKHGNSIVLKIKNDEMYVDITCSVPFNKK |
| | | | | | | ESTINKVVGVDVNIKHMLLATSITDNGNLKGYVNIYKEVINDPDFKKVCNKEEM |
| | | | | | | KYFKDISKYVTFCPLEFDLLFSRVCKQKDIYDENSKMEEAFSNVLNKLKWKFFES |
| | | | | | | GDNKNRIYIENVMKLRSQLKAYCILKNAYYKQQSEYDLGKSEEYIQEHPFSNTE |
| | | | | | | KGKEILSKMDNISQKIIGCRNNIIQYAYNVFEKNGYDMISLEKLTSSQFEKKTMPT |
| | | | | | | VKSLLNYHKILGHTQEEMEKNDIYSVIKKGYYDFTFDNDKVTDAKLSEKGELQK |
| | | | | | | IKDDFFNLMIKAIHFADIKDYFITLSNNGTVGVSLVPSQFTSQMDSIEHKIYFVND |
| | | | | | | KKNGKLKLANKHKVRKSQERHINGLNADYNAANNIAYIVEDETWRNTFMKQS |
| | | | | | | RTDKSLYNKPSYDTSIKSQGSVVSKLKKLGMVKILEAAEV |
| MG191 ancestral | 1858 | MG191-48 | Protein | | | MARHKKKKEGVANKTYCFKVKRMYDMNLETIKKTIDEFDNGYNLCSEALHKIL |
| effector | | ancestral | | | | TEKTIGDLDSYIPESSKEIKGFPLLVTEDYADKPIYAFIEKEVDANKVDNALYWYF |
| | | effector | | | | KKALNGYGETNSLHINESTFRRNGFCKQVGGNYRTKLQQFNNKIKKVDITSKSP |
| | | | | | | YEVILEQCARDVSREHLKNISDFNTKAKILSDKKASGKKELERCELMCKFFKEHE |
| | | | | | | TDVKRKIEELTVEIMPVCGGCRLNSERKVMSVIWGSTSNLTITQISKYDFNFRFDP |
| | | | | | | SSGHFSCDVWGRKDIIKGDDVLVVLYEAMSNNTLFKIVDNTMYIHLSCSAPFKT |
| | | | | | | KTTPIPKMVGCDVNTKHAFLQFDEVNVDDLKGYVNIYQALIEDKAYNELCKKY |
| | | | | | | PKSLYETLSKHACFCPIESQWLMYRAIPRKDRNPIANKFEEVFSQVIDRLLDEAKT |
| | | | | | | KGEHEKVYYLGCVRKFRQKLLSVCRIQTAYDYSGTLYAINKAFPDSESKEKKEL |
| | | | | | | ASHPFNTTEKGKEMLQKKENIRKRIISCRNNIIHYAYSVLAKNGYDCIALENLTT |
| | | | | | | AGLEKKDYMKYPETFLYSKGFLGMTKEEIQKDQYLKKYDEKMYKYDTDKERIE |
| | | | | | | NILFTEKGKKDKEKSDFNNFALKMVHFASVKDYFVTLONKGTVAVAWVPSEYS |
| | | | | | | SQIDSKEHVLYVKDKKKNGKIVPVKKEAVRKKQEDYINGLNADINAANNIRYY |
| | | | | | | VLNNSKFGKFVQKTPAKKLAFNKPSCSPKGDIVKKIQEQYRILNQDELLELEEL |
| MG191 ancestral | 1859 | MG191-49 | Protein | | | MANKKKKEENTITKTFVFKAKMSKNDTMSLWKPAIEEYQNYYNKVSDWICDN |
| effector | | ancestral | | | | LTSMKIGDLAQYIDNKDTAYYKYALSEEYKDKPLYKIFTKGFNSNNADNALYE [symbol] |
| | | effector | | | | IKKTNVDNYNGNILGLSETYYRRNGYVKNVISNYRTKMSTLKVNIKRKKIDEDS |
| | | | | | | VTEETLMEQTIYEIQKNGLKSKKDFEDHIEYLKSKEDPNTNYIERLQTLYDYYKM |
| | | | | | | HKDEINEKMEQMAVEQLKKFGGCRRKNNKSSMTINIQNFKIKKEGSCSYILHLP [symbol] |
| | | | | | | NKKNYNINLWGNRQVVKGKNGERVDLVDIVNKHGDSITFKIKNGELYVTITCD |
| | | | | | | VDFEKKEHEIKNVVGVDVNIKHMLLATSIKDNGNVKGYINIYKELLNDEDFVK [symbol] |
| | | | | | | LNKEELKYYKDLSKYVTFCPIEFDFLFSRVANQKGHNNLNDKLIKREKAFSNVL |
| | | | | | | DKLQKKYKDNDTKIANYINNVKKLRAQYKAYFILKQKYYEKQKEYDLKMGFT |
| | | | | | | DESTESKETMDKRRFENPFINTETAKELLSKMNNVSQDIIGCRDNIITYAYNVFEN |
| | | | | | | NGYDTIALENLESSQFEKRRLPSPKSLLKYHKLQGKTMDEVEKNEKVSKFTKDY |
| | | | | | | YNFEFDDNGKITDIKYSEKGETKKKKSLFYNLVIKAIHFADIKDKFIQLSNNNKV |
| | | | | | | QVVLVPSAFTSQMDSKTHKLYVVETVDKKNKKKLKLVDKRKVRKQQERHING |
| | | | | | | LNADYNAACNIAYIVENEELRNTMCKTPNDKNMYNTPVFNTKTKSQKNVSANII |
| | | | | | | NOLKKMGMTKILELDEAGQFAEN |
| MG191 ancestral | 1860 | MG191-50 | Protein | | | MANKKKKEENTITKTFVFKAKVSKNDIMSLWKPAIEEYVNYYNKVSDWICDNL |
| effector | | ancestral | | | | TSMKIGDLAQYIDNKDSAYYKYVIDEEKKDLPLYKIFTKGFSSQNADNALYCAI |
| | | effector | | | | KKINPENYNGNILGLCETYYRRNGYVKNVISNYRTKMSTLKANIKYKKFDENSV |
| | | | | | | TEETLMEQTIYEVTKHGIESKKDFENLIEYLKNRETPNTEKIERLETLYDYYKKH |
| | | | | | | KDEINEKIEQMAVEQLKKFGGCRRKGNKSTMTIHMQNSKIKKDGLTSYTLHLVL |
| | | | | | | NKKPYDINLLGNRQVVKVKNGNRVDLVDICNKHGDSITFKIKNGELFVTLTSEV |
| | | | | | | DFEKKEHEIKNVVGVDVNIKHMMLATSIVDNGNVKGYINIYKELLNDEDFVSTL |
| | | | | | | NNEELEYYKEMSKYVNFGLLEVDSLFSRVTNQQKNNNLDDKLIKREKAISNVLD |
| | | | | | | KLAKKYRDKDTKIANYIDYTKMLRAKYKAYFILKEKYYEKQKEYDLKMGFTDE |
| | | | | | | STESKETMDKRRFENPFINTETAKELLSKLNNVEQDIIGCRDNIITYAFNVFKNNG |
| | | | | | | YDTIALEYLDSSQFDKRRMPSPKSLLKYHKFEGKTEDEVEKLEKESKFNKEYYN |
| | | | | | | FEFDDNGKITDISYSEEGEREKKKSNFYNLIIKAIHFADIKDKFVQLSNNNKMNV |
| | | | | | | VFVPSAFTSQMDSKTHKLYVVETIDKKNKKKLKLVDKRKVRKQQERHINGLNA |
| | | | | | | DFNAACNIKYIAENEELLNTMCKTKNDKNMYNTPAYNIKEKFKKNVSANIINTL |
| | | | | | | KKMGMVKILEIDEDGQFAEN |
| MG191 ancestral | 1861 | MG191-51 | Protein | | | MAKKKEENTITKSFVFKAKVSKNDIMSLWKPAIEEYVNYYNKVSDWICDNLTS |
| effector | | ancestral | | | | MKIGDLAQYIDNKDSAYYKFVIDEEIKDL.PLYKIFTKNTQNADNALYCAIKKINP |
| | | effector | | | | EGYNGNILGLCETYYRRNGYVKNVISNYRTKMSTLKPNIKYKKFDENSVTEEVL |
| | | | | | | MEQTIYEVVKKGIESKEDFENLIEYLKNRETPNTEKIKRLETLYDYYKKHKDEIN |
| | | | | | | EKIEQMAVEQLKSFNGCRRKGNKSTMTIHMQNSKLKKDGLTSYTLHLCLNKKP |
| | | | | | | YDINLLGNRQVVKVDKNGNRVDLVDICNKHGDSITFQIKNGELFVTLTSEVDFD |
| | | | | | | KKEHEIKNVVGVDVNIKHMMLATSIVDNGNVKGYINIYKELLNDEDFVSTLNNI |
| | | | | | | ELEYYKEMSKYVNFGLLEVDSLFSRVSNQMDKNNSVSKLDDKLLKREKAISNV |
| | | | | | | LDKLAKKYRDKDCKIASYIDYTKMLRAKYKAYFILKOKYYEKQKEYDLKMGF [symbol] |
| | | | | | | DESTNSKETMDKRRFENPFINTETAKELLSKLNNVESDIIGCRDNIINYAFNVFK [symbol] |
| | | | | | | NGYDTIALEYLDSSQFDKRRIPTPKSLLKYHKFEGKTEDEVDKSKFNKKYYNFE [symbol] |
| | | | | | | DDNGKITDISYSEKGLREIKKKNFYNLIIKAIHFADIKDKFVQLSNNNKMNVVFV |
| | | | | | | PSAFTSQMDSKTHKLYVTETIDKKNKKKLKLVDKRKVRKQQERHINGLNADFN |
| | | | | | | AACNIKYIAENEELLNTMCKKKKDKNMYGTPFYNIKDTFKKNVSANIINTLKKM |
| | | | | | | GMVKILELDEA |
| MG191 ancestral | 1862 | MG191-52 | Protein | | | MAKTKESNTITKSFKFKAKVSKNEINSLWKPAIEEYVNYYNKVSDWICDNLTSM |
| effector | | ancestral | | | | KIGDLAQYIDNKDSAYYKFVIDEEIKDLPLYKIFTKSTQNVDNALFCAIKKINPEG |
| | | effector | | | | YNGNILGLCETYYRRNGYVKNVISNYRTKMSTLKPNIKYKKFDENSVTEEVLME |
| | | | | | | QTIYEVVKKGIESKEDFENLIENFKNRETPNTNKIKRLETLYSFFKKHKDEIIEKM |
| | | | | | | EQMAVEQLKSFNGCRRKGNKSTMTIHMQNSKLKKNGLTSFTLHLCLNKKPYDI |
| | | | | | | NLLGNRQVVKVDKNGNRVDLVDICNNHGDYITFQIKNGEMFVILTSEVDFDKKE |
| | | | | | | SEIKNVVGVDVNIKHMMLATSIVDNGNVKGYINIYKELLNDEDFVSTCNNEELE |
| | | | | | | YYKEMSKSVNFGLLEVDSLFSRVSNQMDKNNSVSKLDDKLLKREKAISNVLDR |
| | | | | | | LAKKYRDKDCKIASYIDYTKMLRAKYKAYFILKQKYYEKQKEYDLKMGYTDES |
| | | | | | | TNSKETMDKRRFENPFIDTETAKELLSKLNNIESDLIGCRDNIINYAFNVFKNNGY |
| | | | | | | DTIALEYLDSSQFDKRRIPTPKSLLKYHKFEGKKEDEVDKSKFNKKNYNFEFDDN |
| | | | | | | GKITDISYSERGLREIKKKNFYNLIIKAIHFADIKDKFVQLSNNNKMNIVLVPSAFS |
| | | | | | | SQMDSKTHKLYVTETIDKKNKKKLKLVDKRKVRKQQERHINGLNADFNAACN |
| | | | | | | LKYIAENEELLNTVCKKKKDKNMYGTPFYNIKDAFKKNVSANMINTLKKMGM |
| | | | | | | VKIFDFDEA |
| MG191 ancestral | 1863 | MG191-53 | Protein | | | MANKKKKGENTINKTFIFKAKCEKNDIISLWKPAAEEYCNYYNKLSKWICDNLT |
| effector | | ancestral | | | | SMKIGDLAQYIDNKNSAYYKYVIDEEKKDLPLYKIFTKGFSSMCADNALYCAIK |
| | | effector | | | | KINPENYNGNILGLCESDYRRNGYVKNVISNFRTKMSTLKANIKYKKFDENNVD |
| | | | | | | EETLMEQTIYEVDKHGIESKKDFENLIEYLKTRETPNTEKIERLECLCDYYSKNK |
| | | | | | | DEINEEIETMAIEDLKKFGGCQRKGSKNTLTIHMQNSKIKKDGLTSYTLQLVFNK |
| | | | | | | KPYTINLLGNRQVVKVVNGKRVDLVDICDKHGDSITFNIKNGELFVHLTSPVDFE |
| | | | | | | KKEHEIKNVVGVDVNIKHMMLATSIVDNGNVKGYINIYKELLNDEDFVSTLNNS |
| | | | | | | ELEMYKKMSKNVNFGILEVDSLFERVVNQQKNNNLDNKLIKREKAIQKVLDNL |
| | | | | | | AKTTRDKDTKIANYINYVKMLRAKYKAYFILKEKYYEKQKEYDLKMGFTDEST |
| | | | | | | ESKETMDKRRFENPFINTETAKEILSKLNNVEQDIIGCRDNIVTYAFNVFKNNGY |
| | | | | | | DTIALEYLDSSQFDKRRMPSPKSLLKYHKFEGKTEDEVEKLMKEKKFSKSYYNF |
| | | | | | | EYDNNGVITDIEYSEEGERKQKKSNFMNLIIKAIHFADIKDKFVQLSNNNDMNV [symbol] |
| | | | | | | FVPSAFTSQMDSKTHSLYYVETITKKEKKKYVLANKKSVRTQQETHINGLNAD [symbol] |
| | | | | | | NAACNIKYIAENEELLNKMCKTKNGKNMYNTPAYNIKKKFKKNVSAKVINTLK |
| | | | | | | KLGNVKYGKIDEDGQFVEN |
| MG191 ancestral | 1864 | MG191-54 | Protein | | | MAHKTNNGENTINKTFIFKAKCEKNDIISLWKPAAEEYCNYYNKLSKWIGDNL [symbol] |
| effector | | ancestral | | | | SMKIGDLAQYIDNKNSAYYLAVIDESKKDLPLYKIFQKGFSSLCADNALYCAIK [symbol] |
| | | effector | | | | INPENYNGNILGICESDYRRNGYVQSVISNFRTKMSSLKANVKWKKFDVNNVD [symbol] |
| | | | | | | ETLKTQTIYDVDKYGIESVKDFKELIEVLKTRETPQNDKIARLECLCDYYSKNEE |
| | | | | | | TINNEIETMAIADLQKFGGCQRKGSLNTLTIHKQNSKIEKEGNTSFNLQLVFNKK |
| | | | | | | PYTINLLGNRQVVKFVNGKRVDLIDITEKHGDTITFNIKNGELFVHLTSPVEFEKK |
| | | | | | | VHEIKNAVGVDVNIKHMMLATSIVDNGNVKGYINIYKELLNDDDFVSTLNDSEL |
| | | | | | | EMYRKMSKNVNFGILEVDSLFERVVNQQKGNNLDNKLIRREQAIQKVLDNIVKT |
| | | | | | | TRDKDQRIVNYINYVKMLRAKYKAYFILKEKYYEKQKEYDLMMGFTDESTESK |
| | | | | | | ETMDKRRMEFPFINTETAKEILSKLNNVEQDIIGCRDNIVTYAFNVFKNNGYDTIS |
| | | | | | | LEYLDSSQFDKRRMPTPKSLLKYHKFEGKTEDEVEQLMKEKKFSKSYYNFKYD |
| | | | | | | NNNVVTDIEYSKEGERKQKKSNFMNLIIKAIHFADIKDKFVQLSNNNDMNVVFC |
| | | | | | | PSAFTSQMDSKTHISLYYVEKITKKEKKKYVLANKKSVRTQQETIIINGLNADYN |
| | | | | | | AACNLKYIAENEELLEKMTKTKNGKTMYNTPAYNIKKKFKKNLSAKTINTFKKL |
| | | | | | | GNVKYGKINEDGQFVED |
| MG191 ancestral | 1865 | MG191-55 | Protein | | | MAHKTNNGENTINKTFIFKAKCEKNDIISLWKPAAEEYCNYYNKLSKWIGDNLT |
| effector | | ancestral | | | | SMKIGDLAQYIDNKNSAYYLAVTDESKKDLPLYKIFQKGFSSLCADNALYCAIK |
| | | effector | | | | KINPENYNGNILGIGESDYRRNGYVQSVISNFRTKMSSLKANVKWKKFDVNNVD |
| | | | | | | EETLKTQTIYDVDKYGIESVKDFKELIEVLKTRETPQKEKIARLECLCDYYSKNEE |
| | | | | | | TIKNEIETMAIADLQKFGGCQRKGSLNTLTIHKQNSKIEKEGNTSFNLQLVFNKK |
| | | | | | | PYTINLLGNRQVVKFVNGKRVDLIDITEKHGDWITFNIKNGELFVHLTSPVEFEK |
| | | | | | | KVHEIKNAVGVDVNIKHMMLATSIVDDGNVKGYINLYKELVNDDDFVSTENDS |
| | | | | | | ELEMYRKMSKNVNFGILEVDSLFERVVNQQKGNNLDNKLIRRERAMQKVFDNI |
| | | | | | | VKTTRDKDQRIVDYVNYVKMLRAKYKAYFILKEKYYEKQKEYDLMMGFTDES |
| | | | | | | TESKETMDKRRMEFPFINTETAKEILSKLNNVEQDIIGCRDNIVTYAFNVFKNNG |
| | | | | | | YDTISVEYLDSSQFDKRRMPTPKSLLKYHKFEGKTEDEVEQLMKEKKFSKSYYN |
| | | | | | | FKYDNENVVSDIEYSKEGEWRQKKLNFMNLIIKAIHFADIKDKFVQLCNNNDMN |
| | | | | | | VVFCPSAFTSQMDSKTHSLYYVEKITKKEKKKYVLANKKSVRTQQETHINGLNA |
| | | | | | | DYNAACNLKYIAENEELLEKMTKTKKGKTMYNTPAYNIKKKFKKNLSAKTINT |
| | | | | | | FRKLGNVKYGKINEDGMFVED |
| MG191 ancestral | 1866 | MG191-56 | Protein | | | MANKKKKQENEIVKTFTFKVKNSNGETMDIMKDAIKEYQNYYNKVSDWICDN |
| effector | | ancestral | | | | LTSMTIGDLAQYIPNKKKDTGYYKYALSDEWKDKPLYKIFTKGFNSNNADNVL |
| | | effector | | | | YEAIKKTNVDNYNGNILGLSETYYRRNGYVKNVISNYRTKMSTLKVKIKRKKIL |
| | | | | | | EDSTEETLMEQTIYEIQKNGLKSKKDWEDHIEYLKSKEDPNQNYIERLQTLYDY |
| | | | | | | YKNNKDEINEKMEELSVEQLKKFGGCRRKNNKKSMTINIQNNSFKIKKEGSCGY |
| | | | | | | ILNLPLNKKNFNIDLWGNRQVIKGKNGEDLVDIVNKHGNSITFKIKNNELYVTIT |
| | | | | | | CDVDFEKKESEIKKVVGVDVNIKHMLLATSIKDNGNLKGYVNIYKELLNDEDFT |
| | | | | | | KVLNKEELKYFKDLSKYVTFCPIEFDFLFSRVANQKGHSNLNDKYIKKEKAFSN |
| | | | | | | VLNKLQKKFKDSNDTKERNYIENVKKLRAQYKAYFILKQKYYEKQKEYDLKM |
| | | | | | | GFTDESTESKETMDKRRFENPFTNTETAKELLSKMNNVSQDIIGCRNNIITYAYN |
| | | | | | | VFENNGYDTIALENLESSQFEKHKRLPSVKSLLKYHKLQGKTIDEVEKNEKVSKF |
| | | | | | | TKDYYNFEFDDNGKITDVKYSEKGEMKKKKSLFYNLVIKAIHFADIKDKFIQLSN |
| | | | | | | NGKVQVALVPSEFTSQMDSITHKLYMVKNKKKGKLVLVDKRKVRKTQERHIN |
| | | | | | | GLNADYNAACNIAYIVENEEWRNTFCKKTPNDKNMYNTPVFDTKKKSQSNIINQ |
| | | | | | | LKKLGATKILELDEA |
| MG191 ancestral | 1867 | MG191-57 | Protein | | | MANKKKKTEEIVKSFTFKVKNSDITKDVIQEYKNYYNKVSNWINNNLTSMTIGD |
| effector | | ancestral | | | | LAQYIPNVSKNTGYIKYALSDEWKDKPLYYIFTKEYNSNNANNLLYEAIKNTNV |
| | | effector | | | | DNYNGNILGLSETYYRKNGYVKRVISNYRTKISTLNVKIKRKKVDENSTVETLEE |
| | | | | | | QTMYEINKLGLKTKKDWEEHIEYLKNQENPNQNTIDRMKILYDYFKNNKDTINE |
| | | | | | | KMEELSIEQLKEFGGCKMKNNTNSMTINIQNFKIKKENSLGYILNLPLNKKNFNI |
| | | | | | | ELWGHRQVIKGNKDSYVELVDIVNKHGNSITFKIKNNELYVVITSDTEFEKKETQ |
| | | | | | | FEKIVGVDVNIKHALLVTSEKDNGNLKGYINLYKEVLNNDEFTKLLNKEELKNF |
| | | | | | | KDLSKYVTFCPIEYDFLFSRIDKQKFNDKYTKKEQAFSNVLYDLQKKFKNTNNT |
| | | | | | | KEYNYISCVNKLRAKYKAYFVLKQKYYEKQKEYDIKMGFVDESTESKETMDKR |
| | | | | | | RFENPFTNTEVAKEILSKMNNVQQDINGCRKNIITYAYKVFENNGYDTIALENLE |
| | | | | | | NSNFEKHKPLPSIKSLLKYHKLQGKTIEEVEQNEKVSNLIEKDYYIFQFNENNKII |
| | | | | | | DAKYSEKGYYKVKKALFFNQMIKTLHFASIKDEFIQLSNNGKVQVALVPSEYTS |
| | | | | | | QMDSITHKLYMIKNKKGKLVKVDKRKVRKTQERHINGLNADFNAACNIKYIVE |
| | | | | | | NEEWRNVFCKSPNNKNMYNTPVLDATKKGQSNIINQLKKLGATKLLEMDE |
| MG191 ancestral | 1868 | MG191-58 | Protein | | | MAKKNKKTEEIVKSFKFKVKNSDITKDVIEEYKEYYNRVSNWINNNLTSMTIGDI |
| effector | | ancestral | | | | AQYLPNVSKTTGYIKVALSDEWKDKPMYYLFTKKYNSNHANNLLYEFIKNNNV |
| | | effector | | | | DNYNGNILGLSETYYRKNGYVKLVISNYRTKINTLNVKIKRKKVDENSTVEDIEK |
| | | | | | | QTMYEIIKLGLKKKKDWEEHIEYLKNQENPNQNSINRFKILYDYFNNNEDTINEK |
| | | | | | | MELLSIEQLKEFGGCKMKNNTNSMTINIQNFKIKKENSIGYILNLPLNKKNFNIEL |
| | | | | | | WGHRQIKKGNKDSYVSLVDIINKHGNNIVFKIKNNELYVVISSDTEFEKKETNFE |
| | | | | | | KIVGVDVNIKHALLVTSEKDNGNLKGYINLYKEILNNDEFTSLLNKEELKNFKDI |
| | | | | | | SKYVTFCPIEYDFLFSRIDKMSFNDKYTKKEQVFSKILYDLQKKFKNENNTKEY [symbol] |
| | | | | | | YISCVNKLRAKYIAYFILKQKYYEKQKEYDIKMGFVDDSTESKESMDKRRFENP [symbol] |
| | | | | | | FTNTEVAKEILSKMNNVQQDINGCRKNIITYVYKVFENNGYDTIALENLENSNFF |
| | | | | | | KHKPLPSIKSLLKYHKLONKTIEEVKQNDKVSEFIEKDYYVFTFNENNKIIDAKY |
| | | | | | | EKGYYKVKKALFFNQMIKTLHFASIKDEFIQLSNNGKTQIALVPSEYTSQMDSIT |
| | | | | | | HKLYMIKNDKGKLVKVDKRKVRKTQERHINGLNADFNAACNIKYIVENEEWR [symbol] |
| | | | | | | VFCKSPNNKEYYNTPVLDATKKGQFNIINQLKKLGATKLLEMEK |
| MG191 ancestral | 1869 | MG191-59 | Protein | | | MANKKKKQENEIVKTFTFKVKNSNGATMDIMKDAIKEYQNYYNKVSDWICDN |
| effector | | ancestral | | | | LTSMTIGDLAQYIPDKKKDTGYYKYALSDEWKDKPLYKIFTKGFNSNNADNVL |
| | | effector | | | | YEAIKTVNVDNYNGNILGLSETYYRRNGYVKNVISNYRTKMSTLKVKIKRKNI [symbol] |
| | | | | | | EDSTEDTLMEQTIYEIQKNGLKSTKDWEDQIEYLKSKEDPNQNYLERLQTLYDY |
| | | | | | | YKNNKDEVNEKMEELSVESLKKFGGCRRKNNKKSMTINLØNNSFKIKKEGSNG |
| | | | | | | YILNFPLNKKNFNIDLWGNRQVIKGKNGEELVDIVNKHGNSITFKIKNNELYVNI |
| | | | | | | TCDVPFEKKESEIKKVVGVDVNIKHMLLATSIKDNGNLKGYVNIYKELLNDEDF |
| | | | | | | VKVLNKEELKYFKDLSKYVTFCPIEFDFLFSRVVNQKGHSNLNDKYIKTEEAFSN |
| | | | | | | VLNKLQKKFKDSGDTKERIYIENVMKLRAQLKAYFILKNAYYEQQKEYDLKMG |
| | | | | | | FTDESTESKETMDKRRFENPFTNTEKAKELLSKMNNVSQKIIGCRNNIITYAYNV |
| | | | | | | FENNGYDTIALENLESSQFEKHKRLPSVKSLLKYHKLQGKTIDEVEKNEKVSKFK |
| | | | | | | KGYYNFEFDDNGKITDVKYSEKGEMKKKKSLFYNLVIKAIIIFADIKDKFIQLSN |
| | | | | | | NGKVQVALVPSQFTSQMDSITHKLYMVKNKKKGKLVLVDKRKVRKTQERHIN |
| | | | | | | GLNADYNAACNIAYIVENEEWRNTFCKKTPNDKNMYNTPLFDTKIKSQSNIINK |
| | | | | | | LKKLGATKILELDEA |
| MG191 ancestral | 1870 | MG191-60 | Protein | | | MANKKKKEENEIVKTFTFKVKNSNGVTMDVLKDAIKEYQNYYNKVSDWICDN |
| effector | | ancestral | | | | LTMTIGDLAQYIPDKKKDTGYYKYLISDEWKDKPLYKIFTKGFNSNNADNVLYE |
| | | effector | | | | AIKTVNVDNYTGNILGLSETYYRRNGYVKNVISNYRTKLSKLKVKIKRKNISEDS |
| | | | | | | TEDTLMEQCIYEIEKNGLKSVKDWEDQIEYLKSKDDSNQNYLERMQTLYDYYK |
| | | | | | | NHKDEVNEKMEEMSVESLKKFGGCRRKNNKKSMTIQLQNNSPFKITKEGRNGY |
| | | | | | | ILNFKKNFNIDLWGNRQVIKNGEELVDIVNKHGNSITFKIKNDELYVNITCSVPFE |
| | | | | | | KKESEINKVVGVDVNIKHMLLATSIKDNGNLKGYVNIYKELLNDEDFVKVCNK |
| | | | | | | DELKYFKDLSKYVTFCPIEFDLLFSRVVNQKGYSNKYIKTEEAFSNVLNKLQKKF |
| | | | | | | KESGDNKERIYIENVMKLRAQLKAYFILKNAYYEQQKEYDLTESKERRFENPFS |
| | | | | | | NTEKGKEILSKMNNISKKIIGCRNNIITYAYNVFENNGYDTIALEKLESSQFEKKR |
| | | | | | | MPSVKSLLNYHKLQGMTIDEVEKNEVFSKIKKGYYNFEFDDDKITDVKYSEKGE |
| | | | | | | MQKIKSEFYNLMIKAIHFADIKDYFITLSNNGKVSVALVPSQFTSQMDSIEHKLY |
| | | | | | | MVKNKKKGKLVLVDKRKVRKTQERHINGLNADYNAACNIAYIVENEEWRNTF |
| | | | | | | MKQSPNDKSMYNTPLFDTKIKSQSSIINKLKKLGATKILELDEC |
| MG191 ancestral | 1871 | MG191-61 | Protein | | | MANKKKTEENEIVKTFSFKVKNSNGITMDVLKDAIKEYQNYYNKCSDWICDNL |
| effector | | ancestral | | | | TMTIGDLYQYIPEKKKDTGYAKFLISDEWKDKPLYKIFTKGFNSNNADNALYEA |
| | | effector | | | | KTVNVENYTGNILGLSETYYRRNGYIKNVISNYRTKLSKLKTKIKRKNISEDSTE |
| | | | | | | DTLMEQCIYEIEKNGLKSVKDWEDQIEYLKSKDDSNQNYLERMQTLYDFYKNH |
| | | | | | | KDEVNEKMEEMSVESLAKFGGCRRKNNKKSMSIQLSSNSPFKITQIGRNSFNIKF |
| | | | | | | SKNFNIDVWGRRDVIKNGELLVDIINKHGNSIVFKIKNDELYVDITCSVPFNKKE [symbol] |
| | | | | | | TINKVVGVDVNIKHMLLATSIKDNGNLKGYVNIYKELINDSDFKKVCNKDEMK |
| | | | | | | YFKDLSKYVTFCPIEFDLLFSRVCKQKGINNKNVKMEEAFSNVLNKLKKKFKES |
| | | | | | | GDNKNRIYIENVMKLRSQLKAYCILKNAYYKQQSEYDLTESKEYIQEHPFSNTE |
| | | | | | | KGKEILSKMDNISKKIIGCRNNIIQYAYNIFEKNGYDMIALEKLTSSQFEKKRMPS |
| | | | | | | VKSLLNYHKLLGMTQEEIEKNEVYSVIKKGYYTFEFDDDKITDAKYSEKGELQK |
| | | | | | | IKSDFYNLMIKAIHFADIKDYFITLSNNGTVSVALVPSQFTSQMDSIEHKLYFVNN |
| | | | | | | KKNGKLTLVDKRKVRKTQERHINGLNADYNAANNIAYIVENETWRNTFMKQSP |
| | | | | | | TDKSLYNTPSYDTTIKSQSSIINKLKKLGMYKILEADEA |
| MG191 ancestral | 1872 | MG191-62 | Protein | | | MANKKKDKEGVANKTYCFKVKRMYDMNLETIKKTIDEFDNGYNLCSEALHKIL |
| effector | | ancestral | | | | TEKTIVDLDSYIPESSKEKKGFPLLVTEDYADKPIYAFIEKEVDANKVDNALYWY |
| | | effector | | | | FKKALNGYGETNSLHINESTFRRNGFCKQVGGNYRTKLQQFTQKIKKVDITSKSP |
| | | | | | | YEVILEQCARDVSREHLKNISDTKTFAKILSDKDNVQKKELERCELMCKFFKEHE |
| | | | | | | TDVKRKIEELTVEIMPVFGGCSHDLNSEVMSVIWGSTSNLTITQISKYDFNFRFDP |
| | | | | | | SSGHFSCDVWGRKDIIDDGEVLVVLYEAMSNNTLFKIVDNTMYIHLSCSAPFKT |
| | | | | | | KTTPIPKMVGCDVNTKHAFLQFDEVNVDDLKGYVNIYQALIEDKAYNELCKKY |
| | | | | | | PKSLYETLSKHACFCPIESQWLMYRAIPRKDENGIANKFEEVFSQVIDRLLDEAK |
| | | | | | | TKGEHEKVYYLGCVRKFRQKLLSVCRIQTAYDYSGTLYAIGVNKAFPDSKKELA |
| | | | | | | SHPFNTTEKGKEMLQKKENIRKRIISCRNNIIHYAYSVLAKNGYDCIALENLTTA |
| | | | | | | GLEKDYMKYPETFLYSKGFLGMTKEEIQKDQYLKKLYEKMYKYDTDKEGRIEN |
| | | | | | | ILFTEKGKKDKEKSDFNNFALKMVHFASVKDYFVTLONKGTVAVAWVPSEYSS |
| | | | | | | QIDSKEHVLYVKDKKKNGKIVPVKKEAVRKKQEDYINGLNADINAANNIRYYV |
| | | | | | | LNNSKFGKFVQQSPPEKFLFNKPSCSPTKGGQDYLLDIVKKSEQYRILNQDFS |
| MG191 repeat | 1873 | MG191-1 | Nucleotide | | | CTTGTTGTATATACTCTTTTATAGGTATTAAACAAC |
| sequence | | active repeat | | | | |
| | | orientation | | | | |
| MG191 minimal | 1874 | MG191-12 | Nucleotide | | | GAAGTGCCATTCCGCCTGACCTTAATACGACTCACTATAGATTGTTGTAACTC |
| array | | active | | | | TTATTTTGTATGGAGTAAACAACTGGAGATATCTTGAACCTTGCATCCCCGG |
| | | minimal | | | | AATTGTTGTAACTCTTATTTTGTATGGAGTAAACAACAGGCTAGGTGGAGGC |
| | | array | | | | TCAGTG |
| MG191 PAM | 1875 | MG191-12 | Nucleotide | | | TTR |
| | | PAM | | | | |
| MG119-28 sgRNA | 1876 | MG119- | Nucleotide | | | AGGGCGCGUUGGAGCGCCUUAGUUUGAGGUGCAGAAUCAAAAAAACUGCC |
| | | 28_sg1_15 | | | | ACGAUGGAGGUCGUUUCAGUCUCUGUACACUCAAAAAAUUCACUUGAGAA |
| | | | | | | AUCAAGUGAAUAUCCAAC |
| U67 Spacer | 1877 | U67 Spacer | Nucleotide | | | GTCGAGGCTTGCGACGTGGT |
| U40 Spacer | 1878 | U40 Spacer | Nucleotide | | | TGGAGATATCTTGAACCTTG |
| 6XHis | 1879 | 6xHis | Protein | | | HHHHHH |
| (GS)n | 1880 | (GS)n | Protein | | | GS |
| (GGS)n | 1881 | (GGS)n | Protein | | | GGS |
| (GGGGS)n | 1882 | (GGGGS)n | Protein | | | GGGGS |
| LEVQFQGP | 1883 | PSP | Protein | | | LEVQFQGP |
| ENLYFQG | 1884 | TEV | Protein | | | ENLYFQG |
| KRPAATKKAGQA | 1885 | Nucleoplas | Protein | | | KRPAATKKAGQAKKKK |
| KKKK | | min bipartite | | | | |
| | | NLS | | | | |
| PKKKRKV | 1886 | SV40 NLS | Protein | | | PKKKRKV |
| 119-28 | 1887 | 119-28 | Nucleotide | | | mU*mU*mG*rArArArUrArArArArUrGrArArUrUrUrCrArArArCrCrCrCrUrUrCrGrGr |
| sgRNA1_Mouse_Al | | sgRNA1_ | | | | GrGrGrArGrGrGrCrGrCrGrUrUrGrGrArGrCrGrCrCrUrUrArGrUrUrUrGrArGrGrUrGr |
| b | | ouse Alb | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrArArGrArUrUrGrArUrGrArArGrArCr |
| | | | | | | ArA*mC*mU*mA |
| 119-28 | 1888 | 119-28 | Nucleotide | | | AAGATTGATGAAGACAACTA |
| sgRNA1_Mouse | | sgRNA1_M | | | | |
| Albumin Target | | ouse Album | | | | |
| | | in Target | | | | |
| 119-28 | 1889 | 119-28 | Nucleotide | | | mU*mU*mG*rArArArUrArArArArUrGrArArUrUrUrCrArArArCrCrCrCrUrUrCrGrGr |
| sgRNA2_Mouse_ | | sgRNA2 M | | | | GrGrGrArGrGrGrCrGrCrGrUrUrGrGrArGrCrGrCrCrUrUrArGrUrUrUrGrArGrGrUrGr |
| Alb | | ouse Alb | | | | CrArGrArArUrCrArArArArArArArCrUrGrCrGrArCrGrArUrGrGrArGrGrUrCrGrUrUr |
| | | | | | | UrCrArGrUrCrUrCrUrGrUrArCrArCrUrCrArArArArArArUrUrCrArCrUrUrGrArGrAr |
| | | | | | | ArArUrCrArArGrUrGrArArUrArUrCrCrArArCrGrGrUrCrArGrUrGrArArGrArGrArAr |
| | | | | | | GrA*mA*mC*mA |
| 119-28 | 1890 | 119-28 | Nucleotide | | | GGTCAGTGAAGAGAAGAACA |
| sgRNA2_Mouse_Alb | | sgRNA2_M | | | | |
| Target | | ouse_Alb | | | | |
| | | Target | | | | |
| 119-28 | 1891 | 119-28 | Nucleotide | | | mU*mU*mG*rArArArUrArArArArUrGrArArUrUrUrCrArArArCrCrCrCrUrUrCrGrG |
| sgRNA3_Mouse_ | | sgRNA3_M | | | | GrGrGrArGrGrGrCrGrCrGrUrUrGrGrArGrCrGrCrCrUrUrArGrUrUrUrGrArGrGrUrG |
| Alb | | ouse Alb | | | | CrArGrArArUrCrArArArArArArArCrUrGrCrGrArCrGrArUrGrGrArGrGrUrCrGrUrU |
| | | | | | | UrCrArGrUrCrUrCrUrGrUrArCrArCrUrCrArArArArArArUrUrCrArCrUrUrGrArGrAl |
| | | | | | | ArArUrCrArArGrUrGrArArUrArUrCrCrArArCrArGrUrGrUrArGrCrArGrArGrArGrG |
| | | | | | | ArA*mC*mC*mA |
| 119-28 | 1892 | 119-28 | Nucleotide | | | TCTGTGGAAACAGGGAGAGA [symbol] |
| sgRNA3_Mouse_ | | sgRNA3_ | | | | |
| Alb Target | | Mouse_Alb | | | | |
| | | Target | | | | |
| 119-28 | 1893 | 119-28 | Nucleotide | | | mU*mU*mG*rArArArUrArAr |
| sgRNA4 Mouse_Al | | sgRNA4_ | | | | ArArUrGrArArUrUrUrCrArArArCrCrCrCrUrUrCrGrGrGrGrGrArGrGrGrCrGrCrGrUr |
| b | | Mouse_Alb | | | | UrGrGrArGrCrGrCrCrUrUrArGrUrUrUrGrArGrGrUrGrCrArGrArArUrCrArArArArAr |
| | | | | | | ArArCrUrGrCrGrArCrGrArUrGrGrArGrGrUrCrGrUrUrUrCrArGrUrCrUrCrUrGrUrAr |
| | | | | | | CrArCrUrCrArArArArArArUrUrCrArCrUrUrGrArGrArArArUrCrArArGrUrGrArArUr |
| | | | | | | ArUrCrCrArArCrUrCrUrGrUrGrGrArArArCrArGrGrGrArG*mA*mG*mA |
| 119-28 | 1894 | 119-28 | Nucleotide | | | CTGTGGAAACAGGGAGAGA |
| sgRNA4_Mouse_Al | | sgRNA4_ | | | | |
| b Target | | Mouse_Alb | | | | |
| | | Target | | | | |
| 611F HE | 1895 | 611F HE | Nucleotide | | | TGCACAGATATAAACACTTAACGGG |
| 869R HE | 1896 | 869R HE | Nucleotide | | | GGGCGATCTCACTCTTGTCT |
| 680_HE Taqman | 1897 | 680 HE | Nucleotide | | | FAM-AGCAGAGAGGAACCATTGCCACCTTCAG |
| Probe | | Taqman | | | | |
| | | Probe | | | | |
| 611F NGS | 1898 | 611F NGS | Nucleotide | | | GCTCTTCCGATCTNNNNNTGCACAGATATAAACACTTAACGGG |
| 611F NGS | 1899 | 927R_NGS | Nucleotide | | | GCTCTTCCGATCTNNNNNTTCAGCATTATAACTTACAGGCCT |
| 927R NGS | | | | | | |
| SUMO Polypeptide | 1900 | SUMO | Protein | | | SDSEVNQEAKPEVKPEVKPETHINLKVSDGSSEIFFKIKKTTPLRRLMEAFAKRQ |
| Sequence | | Polypeptide | | | | GKEMDSLRFLYDGIRIQADQTPEDLDMEDNDIIEAHREQI |
| | | Sequence | | | | |
| U40 Spacer Long | 1901 | U40 Spacer | Nucleotide | | | TGGAGATATCTTGAACCTTGCATCCCCGGA |
| Prescisson Protease | 1902 | PSP | Protein | | | LEVLFQGP |
| Site | | | | | | |
| SUMO-Encoding | 1903 | Sumo | Nucleotide | | | TCGGACTCAGAAGTCAATCAAGAAGCTAAGCCAGAGGTCAAGCCAGAAGTC |
| Nucleic Acid | | encoding | | | | AAGCCTGAGACTCACATCAATTTAAAGGTGTCCGATGGATCTTCAGAGATCT |
| | | nucleic acid | | | | TCTTCAAGATCAAAAAGACCACTCCTTTAAGAAGGCTGATGGAAGCGTTCGC |
| | | | | | | TAAAAGACAGGGTAAGGAAATGGACTCCTTAAGATTCTTGTACGACGGTAT [symbol] |
| | | | | | | AGAATTCAAGCTG |
| | | | | | | ATCAGACCCCTGAAGATTTGGACATGGAGGATAACGATATTATTGAGGCTC [symbol] |
| | | | | | | CAGAGAACAGATTGGTGGA |
| 5′ SV40 NLS | 1904 | | Protein | | | MAPKKKRKVGGGGS |
| 3′ SV40 1 NLS | 1905 | | Protein | | | SGGAPKKKRKV |
| 3′ SV40 2 NLS | 1906 | | Protein | | | SGGAPKKKRKV |
| 3′ SV40 3 NLS | 1907 | | Protein | | | SGGAPKKKRKVSGGAPKKKRKVSGGAPKKKRKV |
| 5′ Nucleoplasmin | 1908 | | Protein | | | MKRPAATKKAGQAKKKKGGGGS |
| NLS | | | | | | |
| 3′ Nucleoplasmin | 1909 | | Protein | | | SGGKRPAATKKAGQAKKKK |
| 1 NLS | | | | | | |
| 3′ Nucleoplasmin | 1910 | | Protein | | | SGGKRPAATKKAGQAKKKKSGGKRPAATKKAGQAKKKK |
| 2 NLS | | | | | | |
| 3′ Nucleoplasmin | 1911 | | Protein | | | SGGKRPAATKKAGQAKKKKSGGKRPAATKKAGQAKKKKSGGKRPAATKKAG |
| 3 NLS | | | | | | QAKKKK |
| 5′ c-Myc NLS | 1912 | | Protein | | | MPAAKRVKLDGGGGS |
| 3′ c-Myc 1 NLS | 1913 | | Protein | | | SGGPAAKRVKLD |
| 3′ c-Myc 2 NLS | 1914 | | Protein | | | SGGPAAKRVKLDSGGPAAKRVKLD |
| 3′ c-Myc 3 NLS | 1915 | | Protein | | | SGGPAAKRVKLDSGGPAAKRVKLDSGGPAAKRVKLD |
| Synthetic 5′ Class | 1916 | | Protein | | | MQAAKRPRTTGGGGS |
| 2-1 NLS | | | | | | |
| Synthetic 3′ Class | 1917 | | Protein | | | SGGQAAKRPRTT |
| 2-1 1 NLS | | | | | | |
| Synthetic 3′ Class | 1918 | | Protein | | | SGGQAAKRPRTTSGGQAAKRPRTT |
| 2-1 2 NLS | | | | | | |
| Synthetic 5′ Class | 1919 | | Protein | | | MRAAKRPRTTGGGGS |
| 2-2 NLS | | | | | | |
| Synthetic 3′ Class | 1920 | | Protein | | | SGGRAAKRPRTT |
| 2-2 1 NLS | | | | | | |
| Synthetic 3′ Class | 1921 | | Protein | | | SGGRAAKRPRTTSGGRAAKRPRTT |
| 2-2 2 NLS | | | | | | |
| Synthetic 5′ Class | 1922 | | Protein | | | MRAAKRPRTTGGGGS |
| 2-2 NLS | | | | | | |
| Synthetic 3′ Class | 1923 | | Protein | | | SGGRAAKRPRTT |
| 2-2 1 NLS | | | | | | |
| Synthetic 3′ Class | 1924 | | Protein | | | SGGRAAKRPRTTSGGRAAKRPRTT |
| 2-2 2 NLS | | | | | | |
| Synthetic 5′ Class | 1925 | | Protein | | | MAAAKRSWSMAFGGGGS |
| 3 NLS | | | | | | |
| Synthetic 3′ Class | 1926 | | Protein | | | MSGGAAAKRSWSMAF |
| 31 NLS | | | | | | |
| Synthetic 3′ Class | 1927 | | Protein | | | MSGGAAAKRSWSMAFMSGGAAAKRSWSMAF |
| 3 2 NLS | | | | | | |
| Synthetic 5′ Class | 1928 | | Protein | | | MRAAKRKYFAVGGGGS |
| 4 NLS | | | | | | |
| Synthetic 3′ Class | 1929 | | Protein | | | MSGGRAAKRKYFAV |
| 4 1 NLS | | | | | | |
| Synthetic 3′ Class | 1930 | | Protein | | | MSGGRAAKRKYFAVMSGGRAAKRKYFAV |
| 42 NLS | | | | | | |
| 5′ HCV NLS | 1931 | | Protein | | | MPKPQRKTKRGGGGS |
| 3′ HCV NLS | 1932 | | Protein | | | SGGPPRKKRTVV |
| 3′ hRNP A1 NLS | 1933 | | Protein | | | SGGFGNYNNQSSNFGPMKGGNFGGRSSGPY |
| MG119-1 Direct | 1934 | | Nucleotide | | | GACGCAACTTGCGCGTATGCACAAGTGGAAGT |
| Repeat | | | | | | |
| MG119-1 sgRNA | 1935 | | Nucleotide | | | TAATACGACTCACTATAGTTTACATAGATGTAACACTCGGTAAGGTAAATCC |
| with 15 RAR and 24 | | | | | | GTAAGGATAATCCACCCCGGCGAAATGCCGGCCACTAAAATGCATAAAGCG |
| nt universal spacer | | | | | | CGAAATTGCGCGTATGCACAAGTGGAAGTTGGAGATATCTTGAACCTTGCAT |
| | | | | | | C |
| Direct Repeat | 1936 | | Nucleotide | | | GCCCGCGAAGGGTCTTGGTACTTTCGAC |
| MG126-4 | | | | | | |
| Direct repeat | 1937 | | Nucleotide | | | GTCTCACCGTAGTGAGCAATTTTTATCAGTCGTTAC |
| MG118-1 | | | | | | |
| Direct repeat | 1938 | | Nucleotide | | | GGTGTTGATACCCTATATTCCACGGGTATCTCGACC |
| MG122-4 | | | | | | |
| Direct repeat | 1939 | | Nucleotide | | | CTCGATCTAACCATTTGTATTTTCATTCGTTGCTAT |
| MG120-1 | | | | | | |
| Direct repeat | 1940 | | Nucleotide | | | GGTGTAAGACACCTGAATATAGGAGCCATTGACAGG |
| MG91B-24 | | | | | | |
| Direct repeat | 1941 | | Nucleotide | | | CTTGTAAATACCACCATTTTGTAAGCCTTTTACAGC |
| MG91C-10 | | | | | | |

[symbol]

indicates data missing or illegible when filed

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

1. An engineered nuclease system comprising:
a) an endonuclease comprising a sequence having at least 80% sequence identity to any one of SEQ ID NOs: 57, 31, 1-30, 32-56, 58-325, 420-431, 476-624, 629, 1065-1090, 1114-1118, 1746-1752, and 1842-1872; and
b) an engineered guide polynucleotide configured to form a complex with the endonuclease and to hybridize to a target nucleic acid sequence.

2. The engineered nuclease system of claim 1, wherein the endonuclease comprises a sequence having at least 90% or 100% sequence identity to any one of SEQ ID NOs: 57, 31, 1-30, 32-56, 58-325, 420-431, 476-624, 629, 1065-1090, 1114-1118, 1746-1752, and 1842-1872.

3. (canceled)

4. The engineered nuclease system of claim 1, wherein the engineered guide polynucleotide comprises a crRNA and a tracrRNA.

5. The engineered nuclease system of claim 1, wherein the engineered guide polynucleotide comprises a sequence having at least 90% or 100% sequence identity to any one of SEQ ID NOs: 333-335, 355-357, 410-411, 346-347, 368-369, 412-413, 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, 1091-1113, 1119-1120, 1697-1731, 1807-1836, 1876, and 1887-1894.

6. (canceled)

7. The engineered nuclease system of claim 1, wherein the engineered guide polynucleotide is a single guide nucleic acid or a dual guide nucleic acid.

8. (canceled)

9. The engineered nuclease system of claim 1, wherein the engineered guide polynucleotide is RNA.

10. The engineered nuclease system of claim 1, wherein;
(a) the endonuclease binds non-covalently to the engineered guide polynucleotide;
(b) the endonuclease is covalently linked to the engineered guide polynucleotide; or
(c) the endonuclease is fused to the engineered guide polynucleotide.

11-12. (canceled)

13. The engineered nuclease system of claim 1, further comprising a DNA methyltransferase.

14. The engineered nuclease system of claim 13, wherein the DNA methyltransferase binds non-covalently to the endonuclease.

15. The engineered nuclease system of claim 13, wherein the DNA methyltransferase is fused to the endonuclease in a single polypeptide.

16. The engineered nuclease system of claim 13, wherein the DNA methyltransferase comprises Dmnt3A or Dnmt3L.

17. An engineered nuclease system comprising:
(i) a) an endonuclease comprising a sequence having at least 80% sequence identity to any one of SEQ ID NOs: 6-14; and
b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 333-335 and 355-357;
(ii) a) an endonuclease comprising a sequence having at least 80% sequence identity to ID NO: 15; and
b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 410-411;
(iii) a) an endonuclease comprising a sequence having at least 80% sequence identity to any one of SEQ ID NOs: 16-29; and
b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 346-347, 368-369, and 412-413;
(iv) a) an endonuclease comprising a sequence having at least 80% sequence identity to any one of SEQ ID NOs: 57, 31, 1-30, 32-56, 58-150, 420-431, 476-624, and 629; and
b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 326-332, 336-345, 348-354, 358-367, 414-419, 432, 434, 436, 438, 440, 442, 444, 446, 448, 450, 452, 454, 456, 458, 460, 462, 464, 466, 468, 470, 472, 474, 647-766, and 1697-1731; or
(v) a) an endonuclease comprising a sequence having at least 80% sequence identity to any one of SEQ ID NOs: 1065-1090, 1114-1118, and 1746-1752; and
b) an engineered guide polynucleotide configured to form a complex with the endonuclease and hybridize to a target nucleic acid sequence, the engineered guide polynucleotide comprising a sequence having at least 80% sequence identity to any one of SEQ ID NOs: 1091-1113, 1119-1120, and 1876.

18-31. (canceled)

32. A method for modifying a target nucleic acid sequence comprising contacting the target nucleic acid sequence using the engineered nuclease system of claim 1.

33-37. (canceled)

38. A method of modifying a target nucleic acid sequence in a mammalian cell comprising contacting the mammalian cell using the engineered nuclease system of claim 1.

39. (canceled)

40. A method of modifying TRAC, comprising contacting TRAC using the engineered nuclease system of claim 1, wherein:
a) the endonuclease comprises a sequence having at least 80% sequence identity to any one of SEQ ID NOs: 57, 31, 1-30, 32-56, 58-15030-150, 420-431, 476-624, and 629; and
b) the engineered guide polynucleotide is configured to form a complex with the endonuclease and hybridize to a target nucleic acid sequence, the engineered guide polynucleotide comprising a sequence having at least 80% sequence identity to any one of SEQ ID NOs: 767-798.

41. (canceled)

42. A method of modifying APOA1, comprising contacting APOA1 using the engineered nuclease system of claim 1, wherein:
a) the endonuclease comprises a sequence having at least 80% sequence identity to any one of SEQ ID NOs: 57, 31, 1-30, 32-56, 58-15030-150, 420-431, 476-624, and 629; and
b) the engineered guide polynucleotide is configured to form a complex with the endonuclease and hybridize to a target nucleic acid sequence, the engineered guide polynucleotide comprising a sequence having at least 80% sequence identity to any one of SEQ ID NOs: 831-904, 979-1021, 1219-1237, 1491-1506, and 1663-1669.

43. (canceled)

44. A method of modifying AAVS1, comprising contacting APOA1 using the engineered nuclease system of claim 1, wherein:
a) the endonuclease comprises a sequence having at least 80% sequence identity to any one of SEQ ID NOs: 57, 31, 1-30, 32-56, 58-150, 420-431, 476-624, and 629; and
b) the engineered guide polynucleotide is configured to form a complex with the endonuclease and hybridize to a target nucleic acid sequence, the engineered guide polynucleotide comprising a sequence having at least 80% sequence identity to any one of SEQ ID NOs: 1257-1324, 1523-1562, 1677-1686, 1753-1779, and 1807-1836.

45. (canceled)

46. A method of modifying an albumin gene, comprising contacting the albumin gene using the engineered nuclease system of claim 1, wherein:
a) the endonuclease comprises a sequence having at least 80% sequence identity to any one of SEQ ID NOs: 57, 31, 1-30, 32-56, 58-15030-150, 420-431, 476-624, and 629; and
b) an engineered guide polynucleotide is configured to form a complex with the endonuclease and hybridize to a target nucleic acid sequence, the engineered guide polynucleotide comprising a sequence having at least 80% sequence identity to any one of SEQ ID NOs: 1121-1169, 1393-1441, 1603-1632, 1887, 1889, 1891, and 1892-1894.

47. (canceled)

48. A nucleic acid encoding the engineered nuclease system of claim 1.

49. A cell comprising the engineered nuclease system of claim 1.

50-63. (canceled)
